(19) United States
(12) Patent Application Publication
(10) Pub. No.: US 2025/0099488 A1
(43) Pub. Date: Mar. 27, 2025

(54) ANTI-PARASITE COMPOUNDS

(71) Applicant: AUSTRALIAN NATIONAL UNIVERSITY, Australian Capital Territory (AU)

(72) Inventors: Alexander Gerd MAIER, Australian Capital Territory (AU); Merryn Elizabeth FRASER, Australian Capital Territory (AU); Malcolm Donald MCLEOD, Australian Capital Territory (AU); Blake Nicolas CURTIS, Australian Capital Territory (AU); Charles CLAUDIANOS, Australian Capital Territory (AU); Patrick Andrew YATES, Australian Capital Territory (AU)

(73) Assignee: AUSTRALIAN NATIONAL UNIVERSITY, Australian Capital Territory (AU)

(21) Appl. No.: 18/832,491

(22) PCT Filed: Jan. 24, 2023
(86) PCT No.: PCT/AU2023/050041

§ 371 Date: Jul. 23, 2024

(30) Foreign Application Priority Data
Jan. 24, 2022 (AU) 2022900126

(51) Int. Cl. A61K 31/58; A61K 31/568; A61P 33/04; A61P 33/10

(52) U.S. Cl. CPC A61K 31/58; A61K 31/568; A61P 33/04; A61P 33/10

(57) ABSTRACT

The present invention relates to compounds which are active against parasitic infections, such as protozoan parasite infections (including flagellate parasite infections, ciliate parasite infections, amoeba parasite infections and apicomplexan parasite infections) and helminth infections. The present invention also relates to compositions comprising the compounds, and methods of treating or preventing parasitic infections, such as protozoan parasite infections (including flagellate parasite infections, ciliate parasite infections, amoeba parasite infections and apicomplexan parasite infections) and helminth infections, using the compounds.

The present application claims priority from Australian Provisional Patent Application No. 2022900126 filed 24 Jan. 2022, the entirety of which is incorporated herein by reference.

### FIELD OF THE INVENTION

The present invention relates to compounds which are active against parasitic infections, such as protozoan parasite infections (including flagellate parasite infections, ciliate parasite infections, amoeba parasite infections and apicomplexan parasite infections) and helminth infections. The present invention also relates to compositions comprising the compounds, and methods of treating or preventing parasitic infections, such as protozoan parasite infections (including flagellate parasite infections, ciliate parasite infections, amoeba parasite infections and apicomplexan parasite infections) and helminth infections, using the compounds.

### BACKGROUND

A parasite is an organism that survives at the expense of its host. A class of parasites which is associated with internal infection of humans and animals are endoparasites. Endoparasites include helminths and protozoa.

Helminths are worm-like parasites and include nematodes, flukes and tapeworms. Soil-transmitted helminth infections are among the most common infections in humans worldwide and affect the poorest and most deprived communities. Helminth infection of animals also has a large negative impact on farming systems worldwide. The control of helminths relies heavily on use of chemotherapeutics, but resistance to anthelmintic drugs is widespread and increasing.

Protozoan parasites are unicellular eukaryotic organisms which cause a variety of diseases in humans and animals. Protozoan parasites include flagellates such as Giardia, Trichomonas, Leishmania and trypanosomes, ciliates such as Balantidium coli, amoeba such as Entamoeba histolytica, and apicomplexans such as Toxoplasma, Plasmodium, Eimera, Theileria, Babesia, Sarcocystis, and Cryptosporidium. Infections of humans and animals by protozoan parasites have a significant impact on human health and on farming systems.

For example, the Apicomplexans are a large phylum of unicellular parasitic alveolates, most of which are obligate endoparasites of animals or humans.

An example of an Apicomplexan parasite of humans is some species of the genus Plasmodium which cause the disease malaria. Six species of the genus Plasmodium are the causative agent of malaria in humans. The species of Plasmodium which cause malaria are Plasmodium falciparum, Plasmodium vivax, Plasmodium ovale cuttisi, Plasmodium ovale wallikeri, Plasmodium malariae and Plasmodium knowlesi.

In 2019, there were an estimated 229 million cases of malaria worldwide, and the estimated number of deaths was over 400.000. Globally, the most prominent of the malarial parasites are P. vivax and P. falciparum, with Plasmodium falciparum infection accounting for over 90% of all deaths caused by malaria.

Drug intervention can target three different stages of the Plasmodium life-cycle: the liver stage, the asexual blood stage and the sexual (gametocyte) stage. Liver stage intervention prevents establishment of infection (and hence it can be used for prophylaxis) and can potentially also target Plasmodium species (P. ovale/P. vivax) with dormant stages in the liver (hypnozoites) and hence prevent autologous re-infection/relapses. Asexual blood stage interventions are used as curative treatment as the asexual intra-erythrocytic stages cause the disease. In the sexual (gametocyte) stages, only mature gametocytes are transmission competent, since only they can survive and develop in the midgut of the mosquito.

Current drug therapies for the treatment of Plasmodium spp. infections include the use of combinations of known anti-malarial drugs that work synergistically against multiple life stages of the malarial parasite. These drug therapies are prescribed in a stepwise manner from first-line combination therapies to less common last resort therapies. While there have been many recent developments in anti-malarial drug therapies, treatment regimens over the past decade have been dominated by two major groups of well-known anti-malarial drugs; quinoline derivatives and artemisinin derivatives.

Quinoline derivatives that are useful in the treatment of malaria include, for example, chloroquine, amodiaquine, and primaquine. Chloroquine was, until recently, the most widely used anti-malarial drug, and it is still the first-line drug of choice in most sub-Saharan African countries. However, the emergence of drug-resistant parasitic strains is rapidly decreasing its effectiveness. Amodiaquine is a 4-aminoquinolone anti-malarial drug similar in structure and mechanism of action to chloroquine. Amodiaquine has tended to be administered in areas of chloroquine resistance. Primaquine is a highly active 8-aminoquinolone that is effective against P. falciparum gametocytes but is also active against hypnozoites, the dormant hepatic forms of P. vivax and P. ovale. It is the only known drug to cure both relapsing malaria infections and acute cases. Being effective against multiple life stages of the parasite makes primaquine a highly favourable alternative to chloroquine. However, primaquine is rarely prescribed because it can cause haemolysis, has a high associated risk with pregnancy and is not safe to take for patients with glucose-6-phosphate dehydrogenase deficiency.

Artemisinin derivatives include, for example, Artemether, Artesunate, Dihydroartemisinin, and Arteether. Many of the artemisinin derivatives are used in combination therapy for acute cases of malaria to reduce the emergence of drug resistance.

An example of an Apicomplexan parasite of animals is Apicomplexans of the genus Eimeria and Isospora, which are the causative agent of coccidiosis in animals. Coccidiosis is prevalent in a wide variety of animal species, and affected animals include, for example, fish, cattle, poultry, sheep, goats, dogs, and cats. The organism invades the intestinal mucosa of the animal, resulting in destruction of the intestinal mucosa, and causing diarrhea, fever, inappetence, weight loss, emaciation, and in extreme cases, death.

Toxoplasmosis is a disease that results from infection with the Apicomplexan parasite Toxoplasma gondii.

Treatments of disease caused by parasites is typically through the extensive use of chemotherapeutics. However, the emergence of drug resistant parasites is a potential problem for current frontline chemotherapies, especially for widely used anti-parasitic drugs.

There is a significant need for the development of improved anti-parasitic therapies for the treatment of parasitic infections such as apicomplexan parasite infections.

### SUMMARY OF THE INVENTION

In a first aspect, the present invention provides a compound of Formula (I):

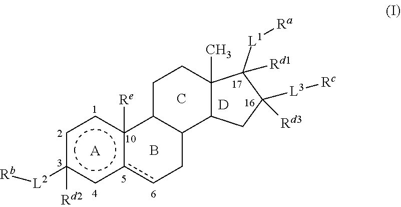

wherein:
each of R^{a}, R^{b} and R^{c} is independently selected from —H, —OH, ═O, substituted or unsubstituted —C₁₋₁₀ alkyl, substituted or unsubstituted —C₂₋₁₀ alkenyl, substituted or unsubstituted —C₂₋₁₀ alkynyl, substituted or unsubstituted —C₃₋₁₀ cycloalkyl, substituted or unsubstituted —C₅₋₁₄ cycloalkenyl, substituted or unsubstituted —C₈₋₁₄ cycloalkynyl, and an anti-parasite moiety; wherein at least one of R^{a}, R^{b} and R^{c} is an anti-parasite moiety;
provided that: when R^{a} is —OH, L¹ is absent; when R^{a} is ═O, L¹ and R^{d1} are absent; when R^{b} is —OH, L² is absent; when R^{b} is ═O, L² is absent, R^{d2} is absent, and the C₂-C₃ bond and the C3-C4 bond are single bonds; when R^{c} is —OH, L³ is absent; when R^{c} is ═O, L³ and R^{d3} are absent;
when R^{a}, R^{b} or R^{c} is substituted, R^{a}, R^{b} or R^{c} is substituted with one or more groups selected from Substituent Group A;
R^{d1}, when present, is H or is a group that, together with R^{a}, forms a substituted or unsubstituted ozonide ring or a substituted or unsubstituted tetraoxane ring, wherein the optional substituent may be a —C₃₋₁₀ cycloalkyl; provided that when R^{d1} and R^{a} form a substituted or unsubstituted ozonide ring or a substituted or unsubstituted tetraoxane ring, L¹ is absent; wherein, when the ozonide or tetraoxane ring is substituted, the ozonide or tetraoxane ring is substituted with one or more —C₃₋₁₀ cycloalkyl groups;
R^{d2}, when present, is H; provided that when R^{d2} is present, the C₂-C₃ bond and the C₃-C₄ bond are single bonds;
R^{d3}, when present, is H or is a group that, together with R^{c}, forms a substituted or unsubstituted ozonide ring or a substituted or unsubstituted tetraoxane ring, wherein the optional substituent may be a —C₃₋₁₀ cycloalkyl; provided that when R^{d3} and R^{c} form a substituted or unsubstituted ozonide ring or a substituted or unsubstituted tetraoxane ring. L³ is absent; wherein, when the ozonide or tetraoxane ring is substituted, the ozonide or tetraoxane ring is substituted with one or more —C₃₋₁₀ cycloalkyl groups;
R^{e} is H or CH₃, or R^{e} is absent;
when present, L¹ is a group that provides a covalent linkage between R^{a} and the C17 of ring D;
when present, L² is a group that provides a covalent linkage between R^{b} and the C3 of ring A;
when present, L³ is a group that provides a covalent linkage between R^{c} and the C16 of ring D;
ring A may be a saturated or unsaturated ring, or ring A may be an aromatic ring, provided that when ring A is an aromatic ring, the C5-C6 bond is a single bond, R^{d2} is absent, R^{e} is absent, and R^{b} is not ═O;
----- is a single bond or a double bond; provided that when the C4-C5 or C5-C10 bond is a double bond, the C5-C6 bond is a single bond; when the C5-C6 bond is a double bond, the C4-C5 and C5-C10 bonds are single bonds; and when the C3 forms a double bond with C2 or C4, R^{b} is not ═O;
or a stereoisomer thereof, or a salt thereof.

The compounds of Formula (I) have anti-parasitic activity. The compounds of Formula (I) are therefore useful in the treatment of parasite infections, such as protozoan parasite infections and helminth infections. Protozoan parasite infections include apicomplexan parasite infections such as malaria, coccidiosis and toxoplasmosis, or flagellate parasite infections such as leishmaniasis.

In a second aspect, the present invention provides a composition comprising a compound of Formula (I), or a salt thereof, and a carrier.

In a third aspect, the present invention provides a pharmaceutical composition comprising a compound of Formula (I), or a pharmaceutically acceptable salt thereof, and a pharmaceutically acceptable carrier, adjuvant or diluent.

In a fourth aspect, the present invention provides a method of treating or preventing a parasite infection in a subject, the method comprising administering to the subject an effective amount of a compound of Formula (I) or a pharmaceutically acceptable salt thereof.

In a fifth aspect, the present invention provides a method of inhibiting the proliferation of a parasite, the method comprising contacting the parasite with an effective amount of a compound of Formula (I) or a salt thereof.

In a sixth aspect, the present invention provides a compound of Formula (I), or a pharmaceutically acceptable salt thereof, for use in treating or preventing a parasite infection in a subject.

In a seventh aspect, the present invention provides the use of a compound of Formula (I), or a pharmaceutically acceptable salt thereof, in the manufacture of a medicament for treating or preventing a parasite infection in a subject.

In an eighth aspect, the present invention provides a method of producing an anti-parasitic compound, comprising coupling a molecule having anti-parasitic activity to a steroid, optionally via a linker, at C3, C16 or C17 of the steroid.

In a ninth aspect, the present invention provides a method of treating or preventing a parasite infection in a subject, the method comprising administering to the subject an effective amount of a compound comprising an anti-parasitic compound coupled to a steroid, optionally via a linker, at C3, C16 or C17 of the steroid.

In various aspects and embodiments, the present invention also provides the following items 1 to 46:
1. A method of treating or preventing a parasite infection in a subject, the method comprising administering to the subject an effective amount of a compound of Formula (I):

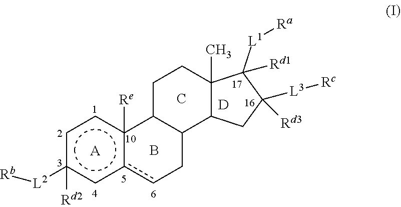

wherein:
each of R^{a} and R^{c} is independently selected from —H, —OH, ═O, substituted or unsubstituted —C₁₋₁₀ alkyl, substituted or unsubstituted —C₂₋₁₀ alkenyl, substituted or unsubstituted —C₂₋₁₀ alkynyl, substituted or unsubstituted —C₃₋₁₀ cycloalkyl, substituted or unsubstituted —C₅₋₁₄ cycloalkenyl, substituted or unsubstituted —C₈₋₁₄ cycloalkynyl, and an anti-parasite moiety; wherein at least one of R^{a} and R^{c} is an anti-parasite moiety;
R^{b} is selected from —H, —OH, ═O, substituted or unsubstituted —C₁₋₁₀ alkyl, substituted or unsubstituted —C₂₋₁₀ alkenyl, substituted or unsubstituted —C₂₋₁₀ alkynyl, substituted or unsubstituted —C₃₋₁₀ cycloalkyl, substituted or unsubstituted —C₅₋₁₄ cycloalkenyl, and substituted or unsubstituted —C₈₋₁₄ cycloalkynyl;
provided that: when R^{a} is —OH, L¹ is absent; when R^{a} is ═O, L¹ and R^{d1} are absent; when R^{b} is —OH, L² is absent; when R^{b} is ═O, L² is absent, R^{d2} is absent, and the C2-C3 bond and the C3-C4 bond are single bonds; when R^{c} is —OH, L³ is absent; when R^{c} is ═O, L³ and R^{d3} are absent;
when R^{a}, R^{b} or R^{c} is substituted, R^{a}, R^{b} or R^{c} is substituted with one or more groups selected from Substituent Group A;
R^{d1}, when present, is H or is a group that, together with R^{a}, forms a substituted or unsubstituted ozonide ring or a substituted or unsubstituted tetraoxane ring, wherein the optional substituent may be a —C₃₋₁₀ cycloalkyl; provided that when R^{d1} and R^{a} form a substituted or unsubstituted ozonide ring or a substituted or unsubstituted tetraoxane ring, L¹ is absent; wherein, when the ozonide or tetraoxane ring is substituted, the ozonide or tetraoxane ring is substituted with one or more —C₃₋₁₀ cycloalkyl groups;
R^{d2}, when present, is H; provided that when R^{d2} is present, the C2-C3 bond and the C3-C4 bond are single bonds;
R^{d3}, when present, is H or is a group that, together with R^{c}, forms a substituted or unsubstituted ozonide ring or a substituted or unsubstituted tetraoxane ring, wherein the optional substituent may be a —C₃₋₁₀ cycloalkyl; provided that when R^{d3} and R^{c} form a substituted or unsubstituted ozonide ring or a substituted or unsubstituted tetraoxane ring, L³ is absent; wherein, when the ozonide or tetraoxane ring is substituted, the ozonide or tetraoxane ring is substituted with one or more —C₃₋₁₀ cycloalkyl groups;
R^{e} is H or CH₃, or R^{e} is absent;
when present, L¹ is a group that provides a covalent linkage between R^{e} and the C17 of ring D;
when present, L² is a group that provides a covalent linkage between R^{b} and the C3 of ring A;
when present, L³ is a group that provides a covalent linkage between R^{c} and the C16 of ring D;
ring A may be a saturated or unsaturated ring, or ring A may be an aromatic ring, provided that when ring A is an aromatic ring, the C5-C6 bond is a single bond, R^{d2} is absent, R^{e} is absent, and R^{b} is not ═O;
[symbol] a single bond or a double bond; provided that when the C4-C5 or C5-C10 bond is a double bond, the C5-C6 bond is a single bond; when the C5-C6 bond is a double bond, the C4-C5 and C5-C10 bonds are single bonds; and when the C3 forms a double bond with C2 or C4, R^{b} is not ═O;
or a stereoisomer thereof, or a pharmaceutically acceptable salt thereof.
2. A method according to item 1, wherein Formula (I) is selected from:

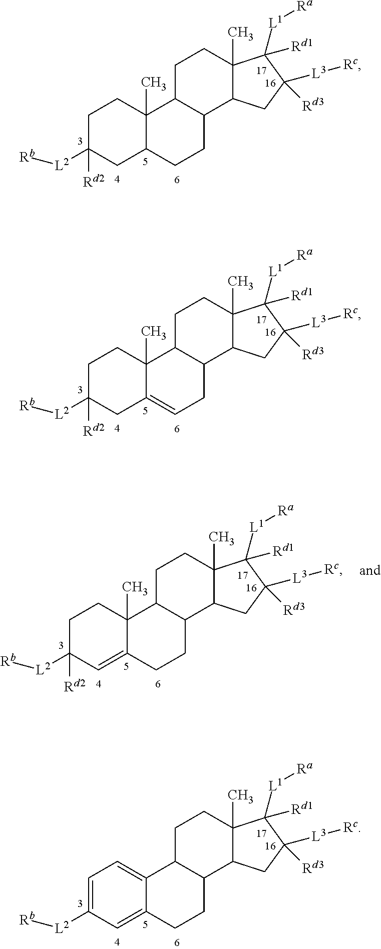

3. A method according to item 1 or 2, wherein Formula (I) is selected from:

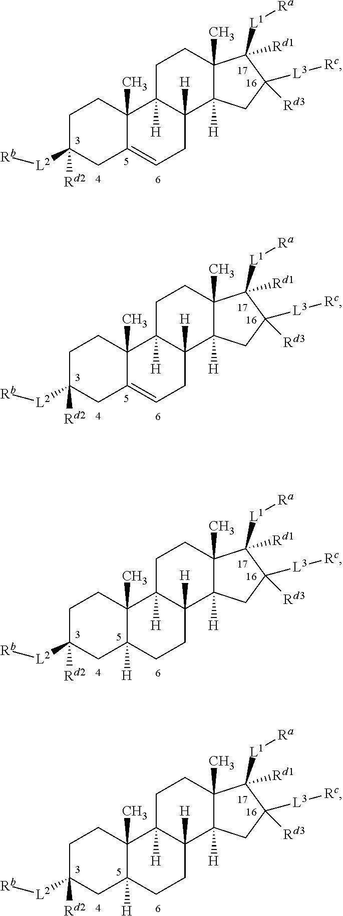

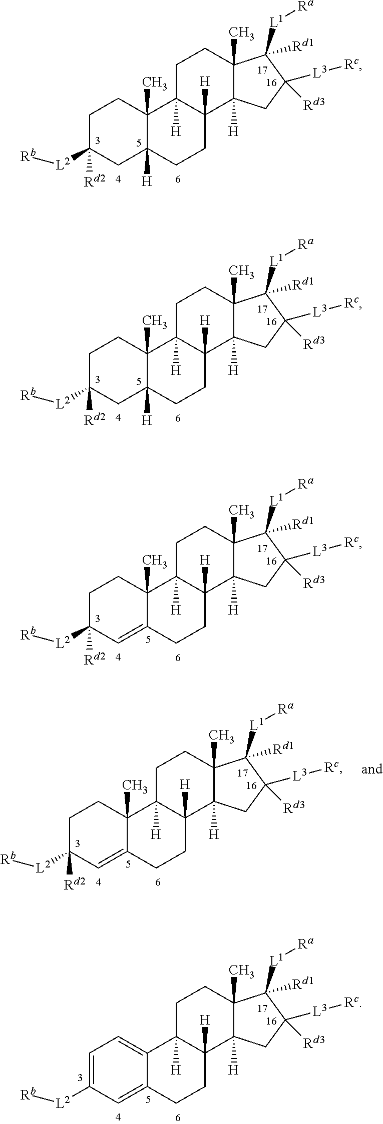

4. A method according to any one of items 1 to 3, wherein the anti-parasite moiety has anti-apicomplexan activity or antihelminth activity.
5. A method according to any one of items 1 to 4, wherein the anti-parasite moiety is an anti-apicomplexan moiety.
6. A method according to any one of items 1 to 5, wherein the anti-parasite moiety R^{a} or R^{c} is selected from:

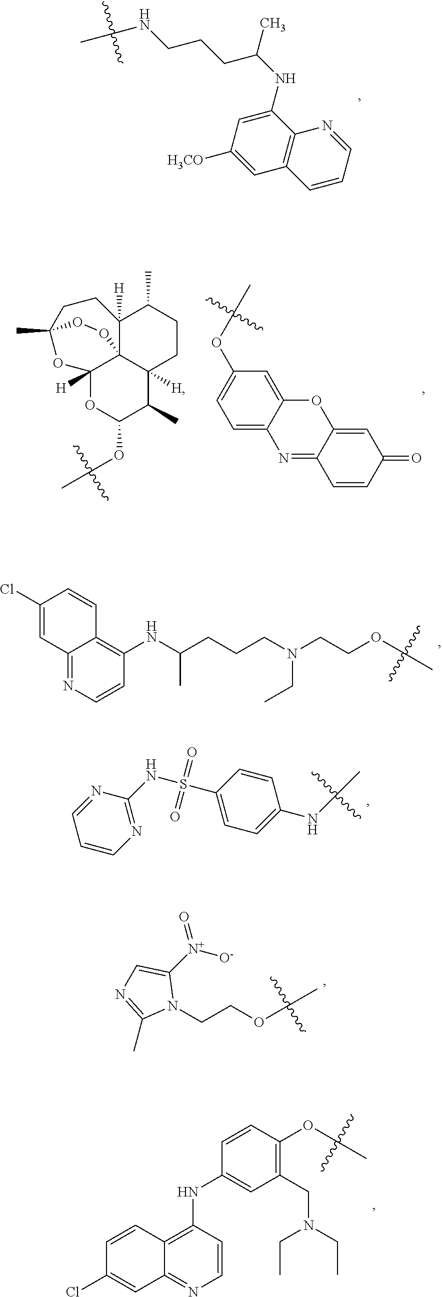

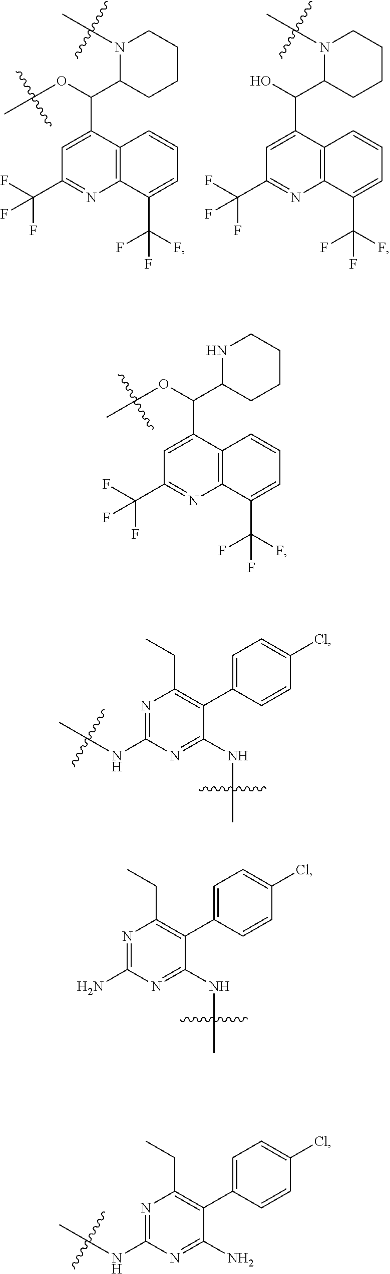

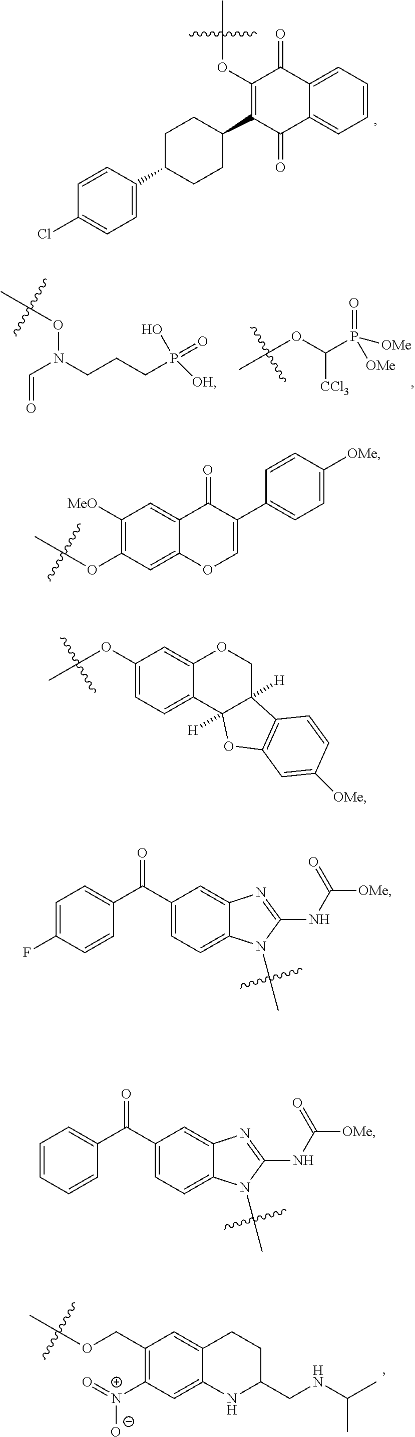

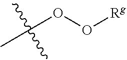

wherein R^{g} is H or substituted or unsubstituted —C₁₋₆ alkyl,

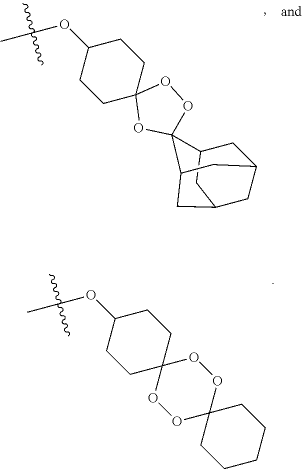 [SMILES: *OC1CCC2(CC1)OOC1(O2)C2CC3CC(C2)CC1C3 ; *OC1CCC2(CC1)OOC1(CCCCC1)OO2]

7. A method according to any one of items 1 to 6, wherein each of L¹, L² and L³, when present, is substituted or unsubstituted and is independently selected from:

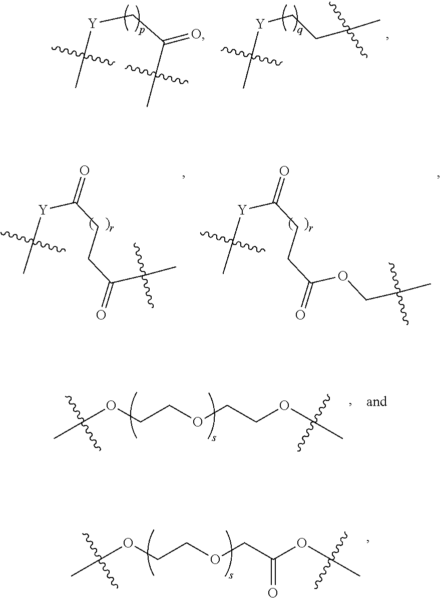

wherein Y is —NR^{f}—, —S—, —O— or —CR^{f1}R^{f2}—, wherein each of R^{f}, R^{f1} and R^{f2} is H or substituted or unsubstituted —C₁₋₆ alkyl; each of p, q, r and s is independently 0, 1, 2, 3 or 4;
wherein, when L¹, L² or L³ is substituted, L¹, L² or L³ is substituted with one or more groups selected from Substituent Group A.
8. A method according to any one of items 1 to 7, wherein each of L¹, L² and L³, when present, is substituted or unsubstituted and is independently selected from:

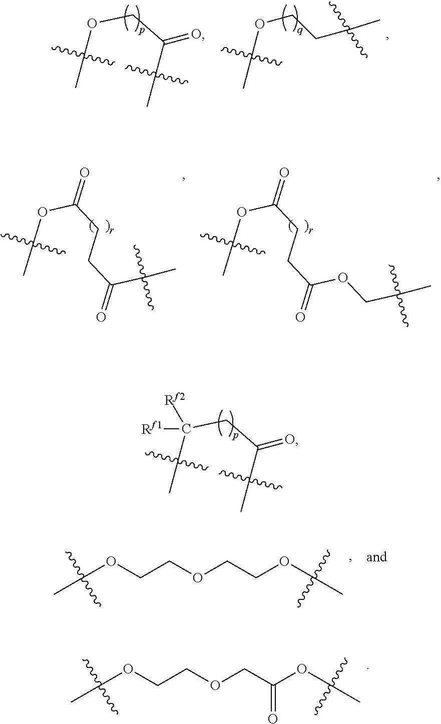

9. A method according to any one of items 1 to 8, wherein each of L¹, L² and L³, when present, is substituted or unsubstituted and is independently selected from:

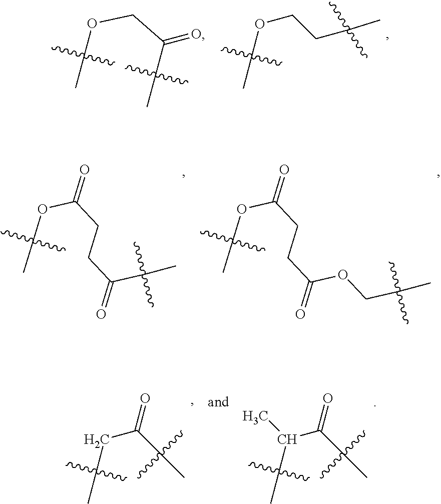

10. A method according to any one of items 1 to 9, wherein the compound of Formula (I) is a compound of Formula (Ia):

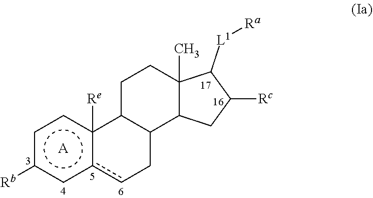

wherein:
R^{a} is selected from:

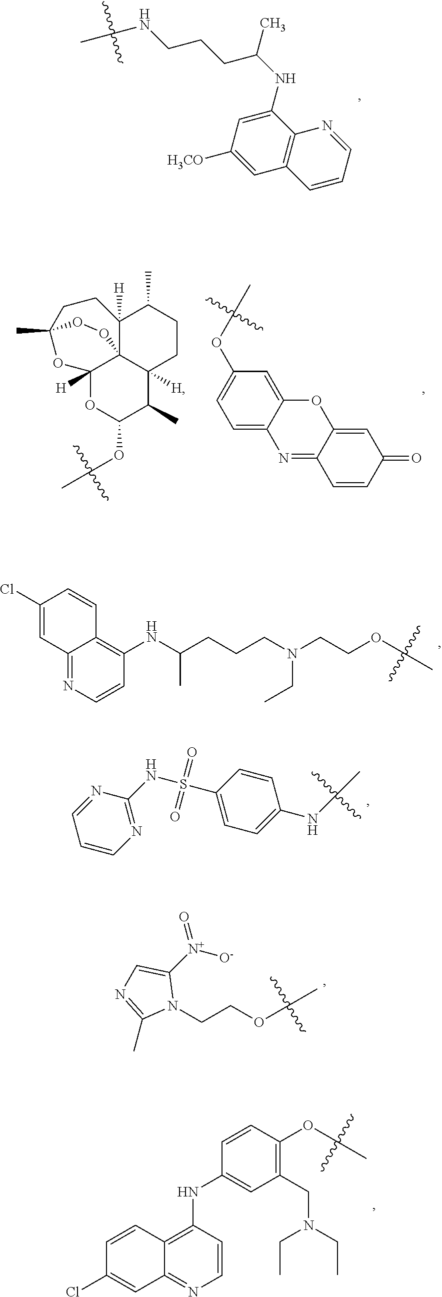

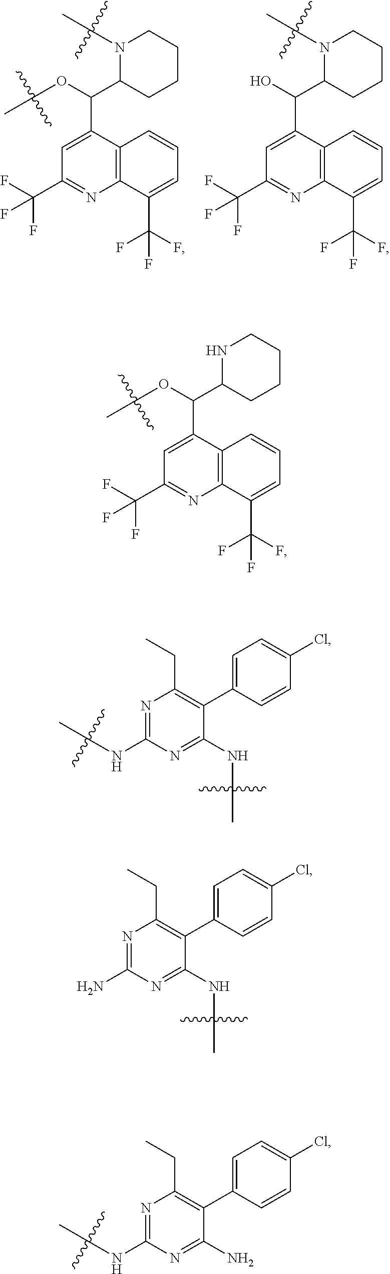

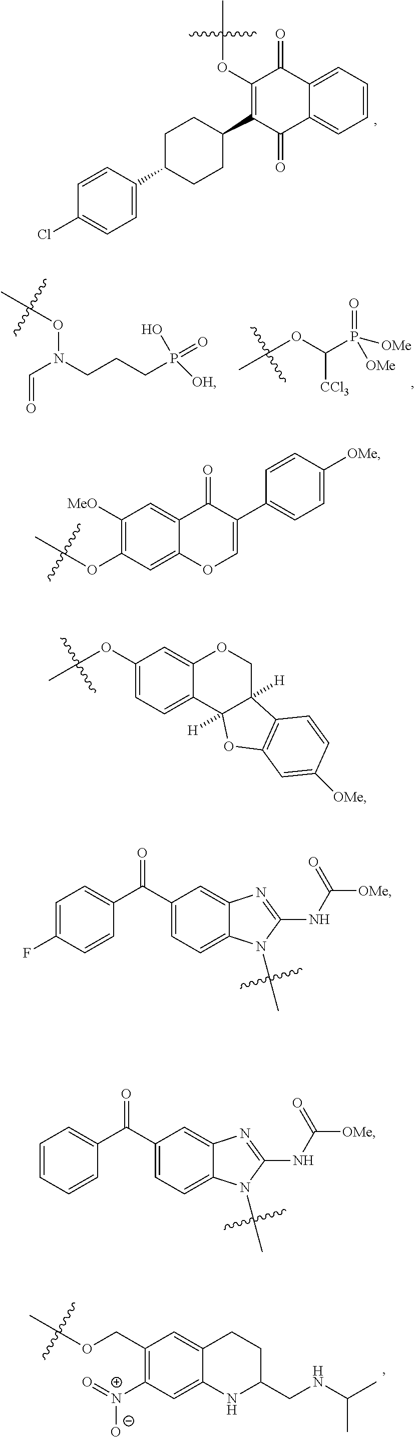

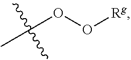

wherein R^{g} is H or substituted or unsubstituted —C₁₋₆ alkyl,

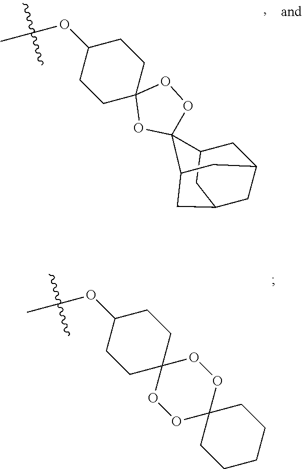 [SMILES: *OC1CCC2(CC1)OOC1(O2)C2CC3CC(C2)CC1C3 ; *OC1CCC2(CC1)OOC1(CCCCC1)OO2]

each of R^{b} and R^{c} is selected from —H, —OH, ═O, substituted or unsubstituted —C₁₋₁₀ alkyl, substituted or unsubstituted —C₂₋₁₀ alkenyl, substituted or unsubstituted —C₂₋₁₀ alkynyl, substituted or unsubstituted —C₃₋₁₀ cycloalkyl; substituted or unsubstituted —C₅₋₁₄ cycloalkenyl, or substituted or unsubstituted —C₈₋₁₄ cycloalkynyl, wherein, when R^{b} or R^{c} is substituted, R^{b} or R^{c} is substituted with one or more groups selected from Substituent Group A;
R^{e} is H or CH₃, or R^{e} is absent;
L¹ may be substituted or unsubstituted and is independently selected from:

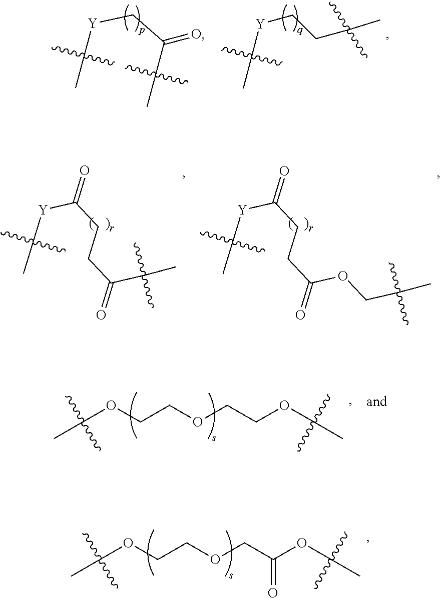

wherein Y is —NR^{f}—, —S—, —O— or —CR^{f1}R^{f2}—, wherein each of R^{f}, R^{f1} and R^{f2} is H or substituted or unsubstituted —C₁₋₆ alkyl; each of p, q, r and s is independently 0, 1, 2, 3 or 4; wherein, when L¹ is substituted, L¹ is substituted with one or more groups selected from Substituent Group A;
ring A may be a saturated or unsaturated ring, or ring A may be an aromatic ring, provided that when ring A is an aromatic ring, the C5-C6 bond is a single bond. R^{e} is absent, and R^{b} is not ═O;
[symbol] is a single bond or a double bond; provided that when the C4-C5 or C5-C10 bond is a double bond, the C5-C6 bond is a single bond; when the C5-C6 bond is a double bond, the C4-C5 and C5-C10 bonds are single bonds; and when the C3 forms a double bond with C2 or C4, R^{b} is not ═O;
or a stereoisomer thereof, or a pharmaceutically acceptable salt thereof.
11. A method according to any one of items 1 to 9, wherein the compound of Formula (I) is a compound of Formula (Ic):

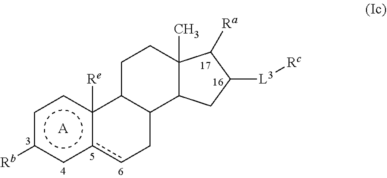

wherein:
each of R^{a} and R^{b} is selected from —H, —OH, ═O, substituted or unsubstituted —C₁₋₁₀ alkyl, substituted or unsubstituted —C₂₋₁₀ alkenyl, substituted or unsubstituted —C₂₋₁₀ alkynyl, substituted or unsubstituted —C₃₋₁₀ cycloalkyl; substituted or unsubstituted —C₅₋₁₄ cycloalkenyl, or substituted or unsubstituted —C₈₋₁₄ cycloalkynyl, wherein, when R^{a} or R^{b} is substituted, R^{a} or R^{b} is substituted with one or more groups selected from Substituent Group A;
R^{c} is selected from:

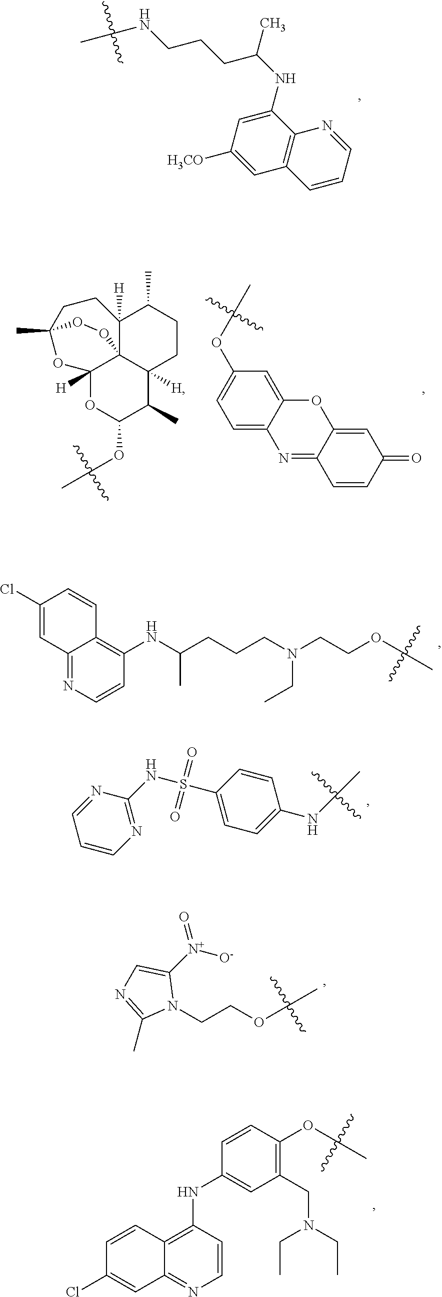

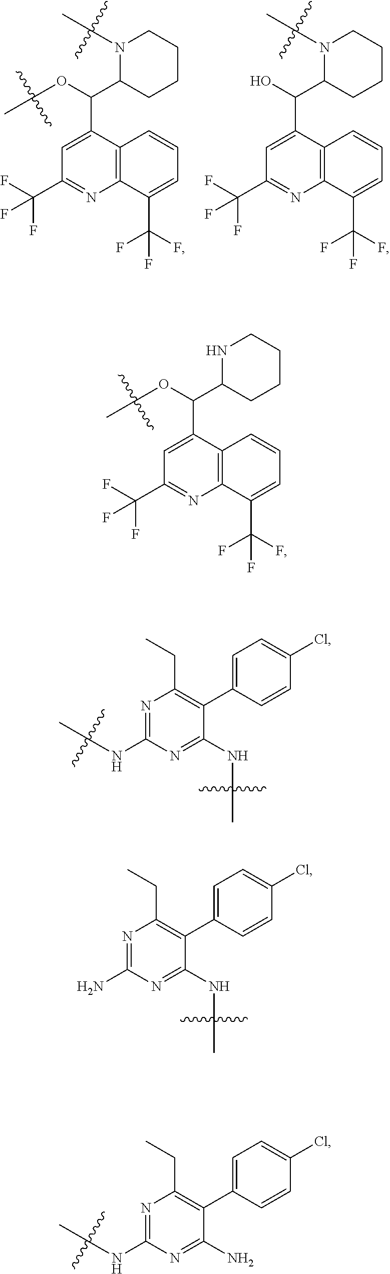

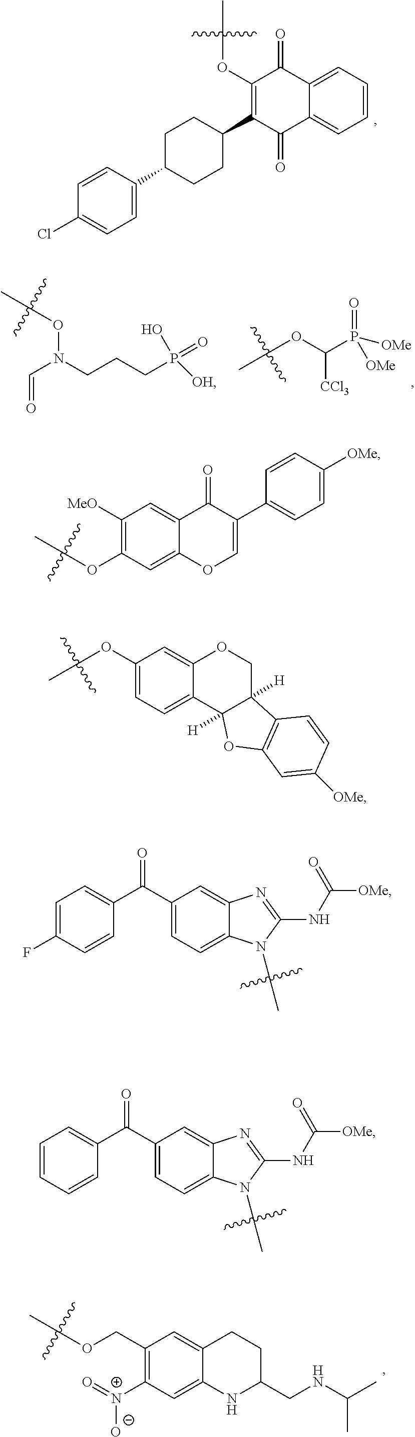

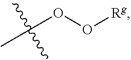

wherein R^{g} is H or substituted or unsubstituted —C₁₋₆ alkyl,

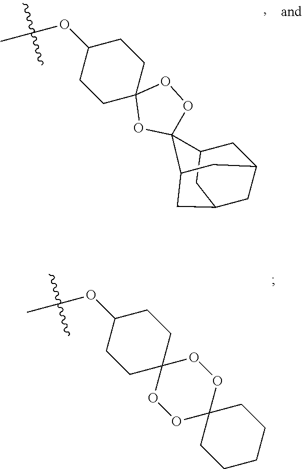 [SMILES: *OC1CCC2(CC1)OOC1(O2)C2CC3CC(C2)CC1C3 ; *OC1CCC2(CC1)OOC1(CCCCC1)OO2]

R^{e} is H or CH₃, or R^{e} is absent;
L³ may be substituted or unsubstituted and is independently selected from:

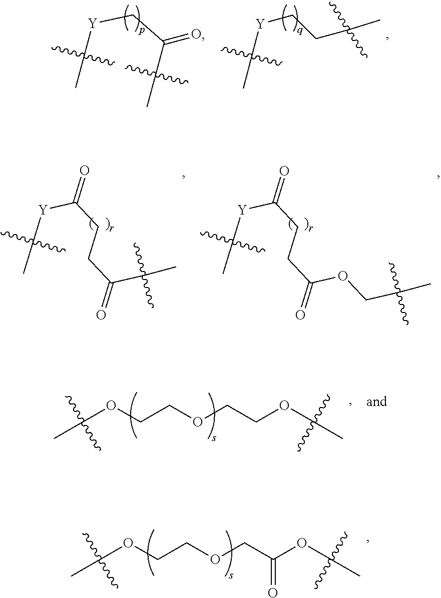

wherein Y is —NR^{f}—, —S—, —O— or —CR^{f1}R^{f2}—, wherein each of R^{f}, R^{f1} and R^{f2} is H or substituted or unsubstituted —C₁₋₆ alkyl; each of p, q, r and s is independently 0, 1, 2, 3 or 4; wherein, when L³ is substituted, L³ is substituted with one or more groups selected from Substituent Group A;
ring A may be a saturated or unsaturated ring, or ring A may be an aromatic ring, provided that when ring A is an aromatic ring, the C5-C6 bond is a single bond, R^{e} is absent, and R^{b} is not ═O;
[symbol] is a single bond or a double bond; provided that when the C4-C5 or C5-C10 bond is a double bond, the C5-C6 bond is a single bond; when the C5-C6 bond is a double bond, the C4-C5 and C5-C10 bonds are single bonds; and when the C3 forms a double bond with C2 or C4, R^{b} is not ═O;
or a stereoisomer thereof, or a pharmaceutically acceptable salt thereof.
12. A method according to item 10 or 11, wherein Formula (Ia) or Formula (Ic) comprises a steroid group selected from:

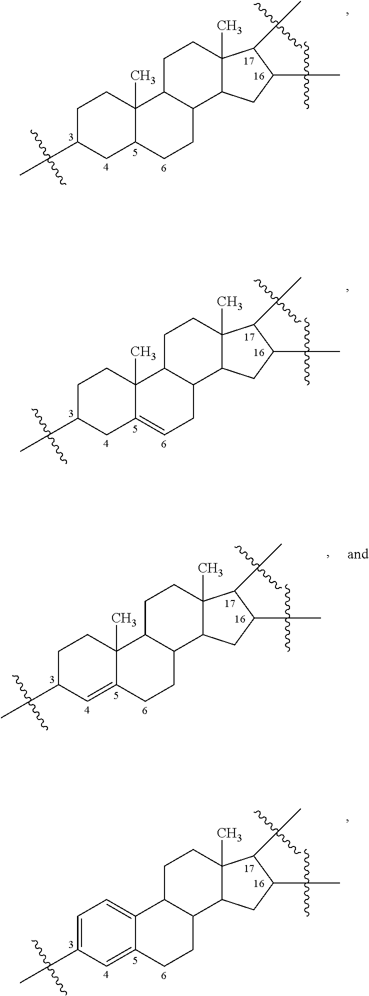 [SMILES: *C1CCC2(C)C(CCC3C2CCC2(C)C(*)C(*)CC32)C1 ; *C1CCC2(C)C(=CCC3C2CCC2(C)C(*)C(*)CC32)C1 ; *C1C=C2CCC3C(CCC4(C)C(*)C(*)CC34)C2(C)CC1 ; *c1ccc2c(c1)CCC1C2CCC2(C)C(*)C(*)CC12]

wherein the groups at C3, C16 and C17 may be attached in the α or β configuration on the respective ring.
13. A method according to any one of items 10 to 12, wherein each of L¹ and L³, when present, is substituted or unsubstituted and is independently selected from:

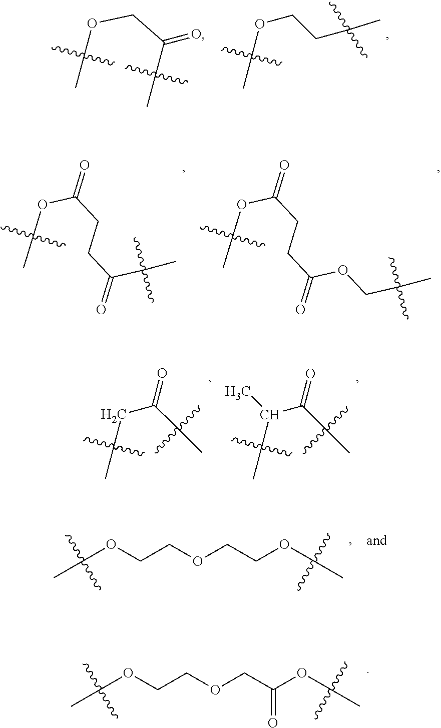

14. A method according to any one of items 10 to 13, wherein R^{a} or R^{c} is selected from:

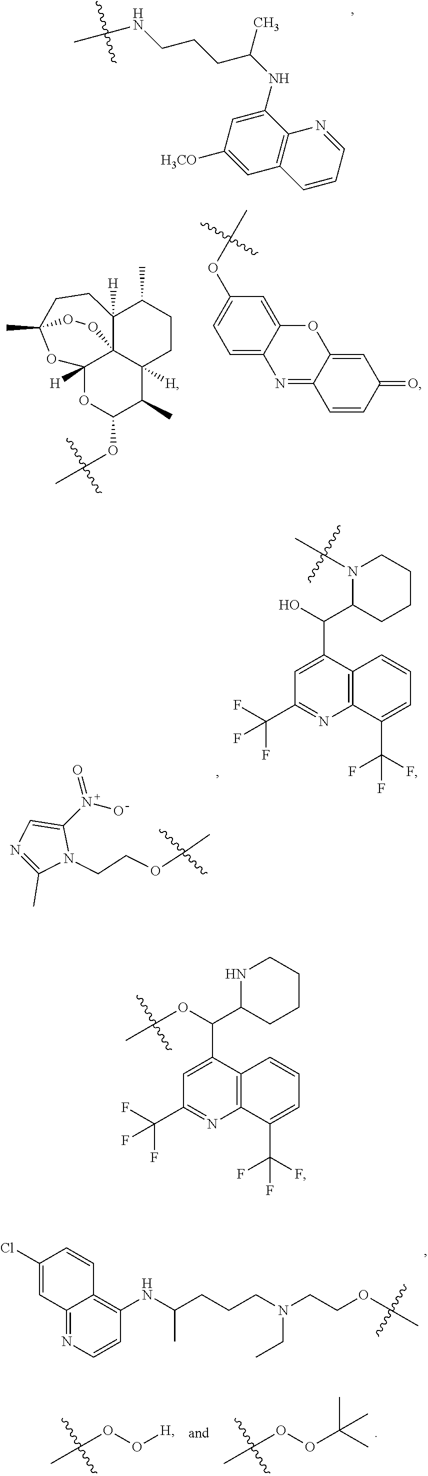

15. A method according to item 1, wherein the compound is selected from:

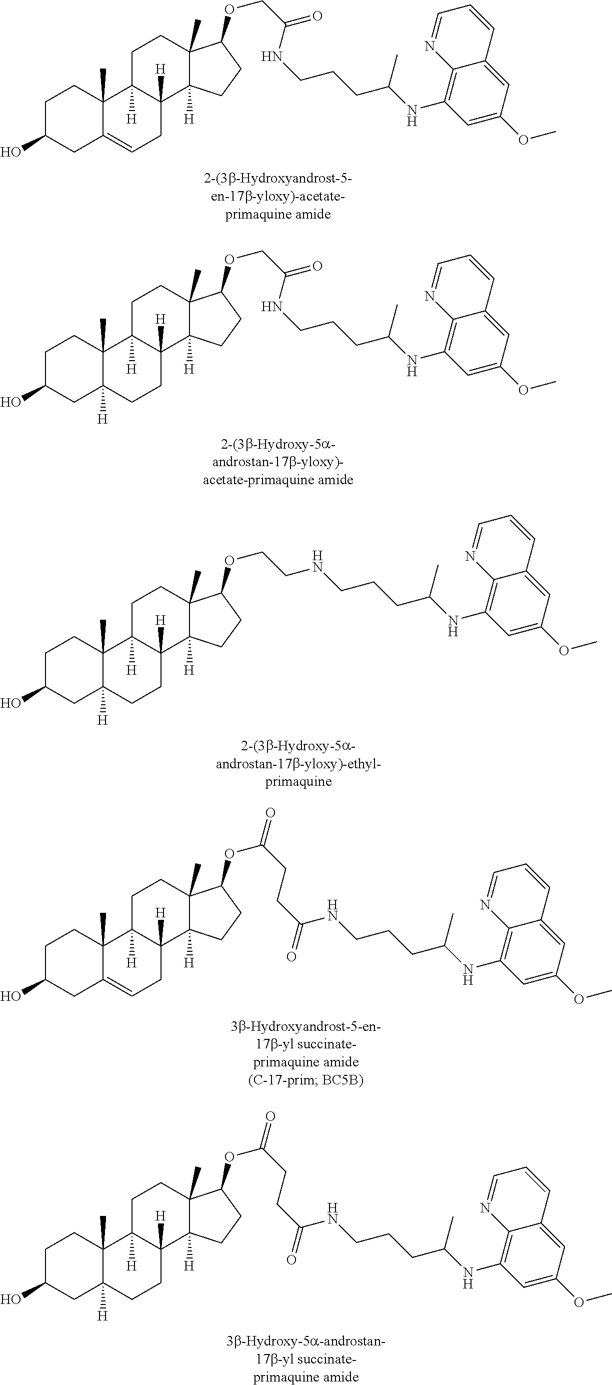

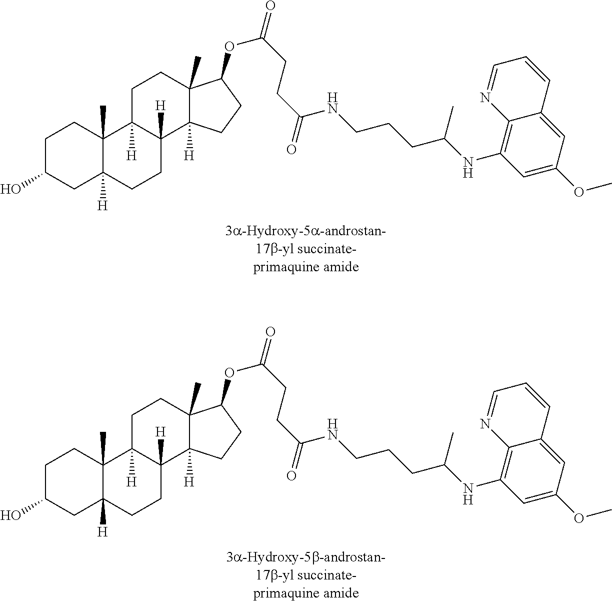

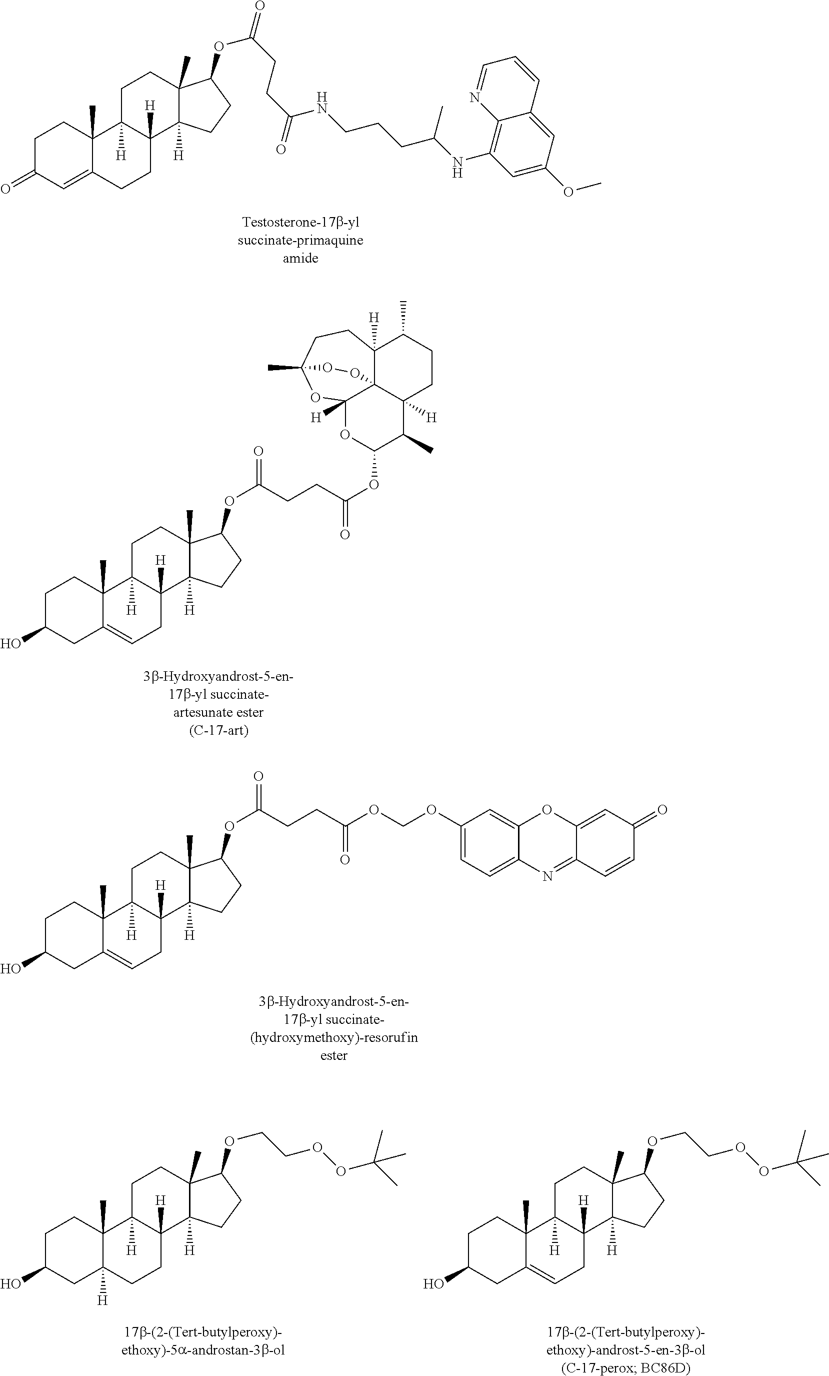

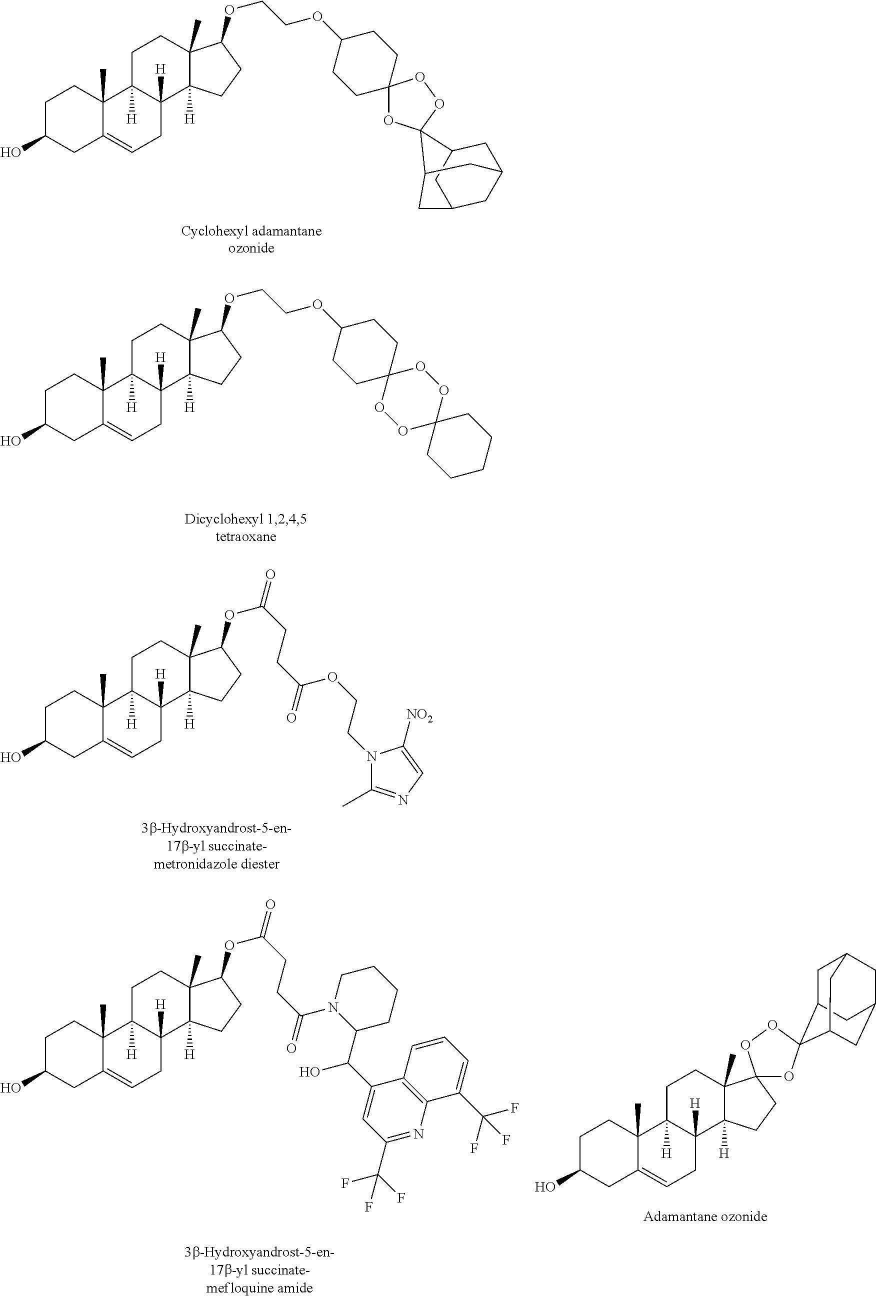

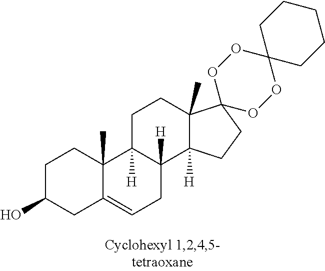

or a pharmaceutically acceptable salt thereof.
16. A method according to any one of items 1 to 15, wherein the parasite infection is an apicomplexan parasite infection or a Leishmania parasite infection.
17. A method according to item 16, wherein the apicomplexan parasite infection is caused by an organism selected from Plasmodium spp., Toxoplasma spp., Eimeria spp., Isospora spp., Theileria spp., Babesia spp., Sarcocystis spp., and Cryptosporidium spp.; or wherein the Leishmania parasite infection is caused by Leishmania tarentolae.
18. A method according to item 17, wherein the apicomplexan parasite infection is caused by Plasmodium falciparum, Plasmodium vivax, Plasmodium ovale curtisi, Plasmodium ovale wallikeri, Plasmodium malariae, Plasmodium knowlesi or coccidia.
19. A method according to item 17, wherein the apicomplexan parasite infection is caused by Eimeria spp. or Isospora spp.
20. A method according to item 17, wherein the apicomplexan parasite infection is caused by Toxoplasma gondii.
21. A method according to any one of items 1 to 3, wherein the anti-parasite moiety has anti-protozoan activity or antihelminth activity.
22. A method according to item 21, wherein the anti-parasite moiety is an anti-protozoan moiety.
23. A method according to item 21 or 22, wherein the anti-parasite moiety is an anti-apicomplexan moiety.
24. A method according to any one of items 21 to 23, wherein any one or more of the following apply:
   (a) R^{a} or R^{c} is selected from:

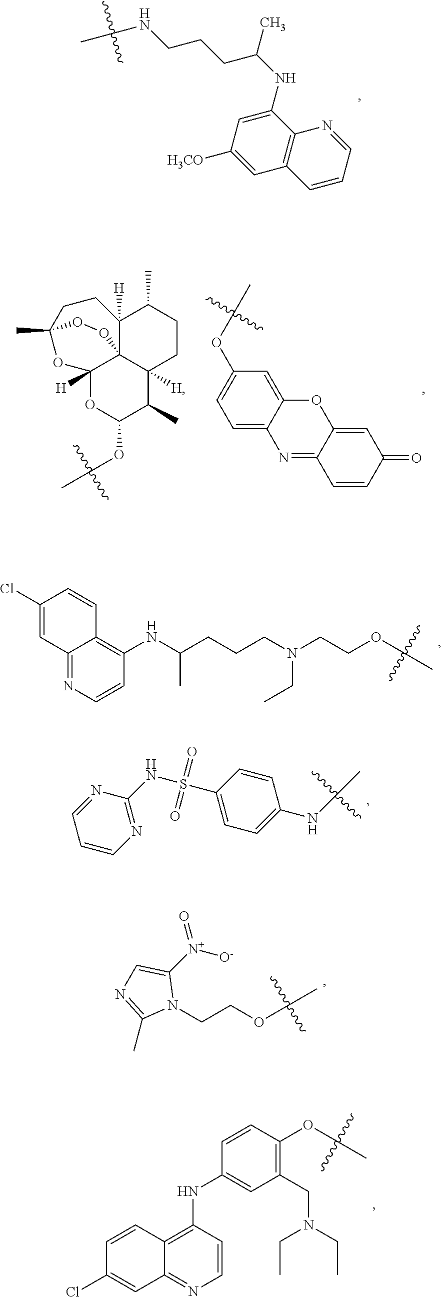

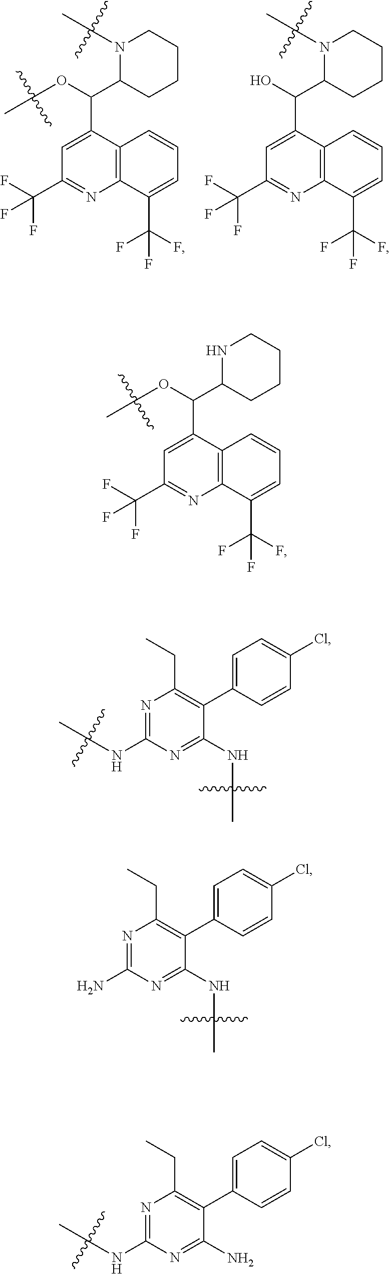

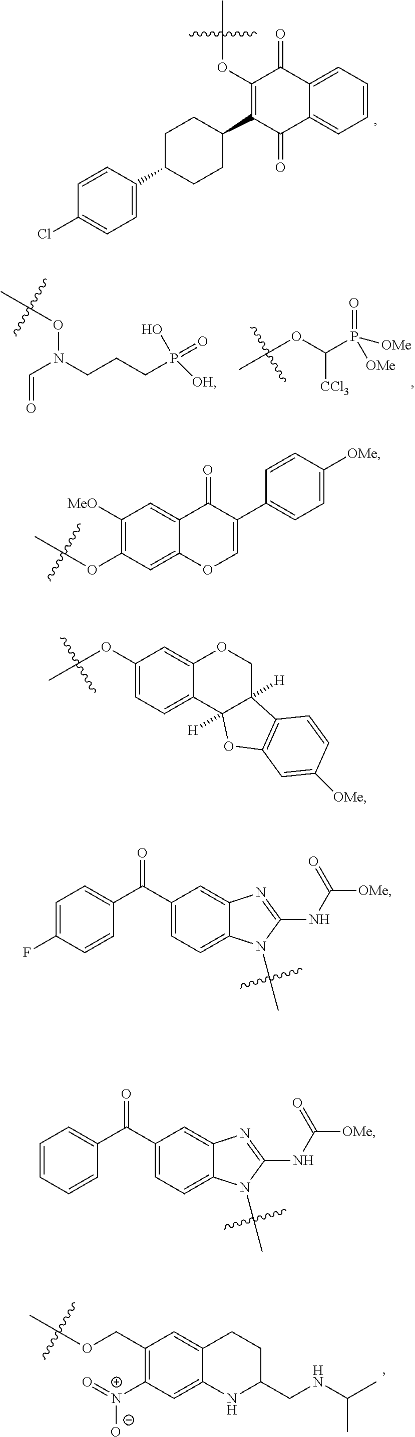

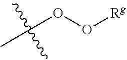

wherein R^{g} is H or substituted or unsubstituted —C₁₋₆ alkyl,

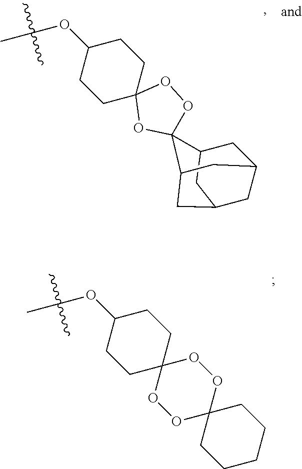 [SMILES: *OC1CCC2(CC1)OOC1(O2)C2CC3CC(C2)CC1C3 ; *OC1CCC2(CC1)OOC1(CCCCC1)OO2]

(b) each of L¹, L² and L³, when present, is substituted or unsubstituted and is independently selected from:

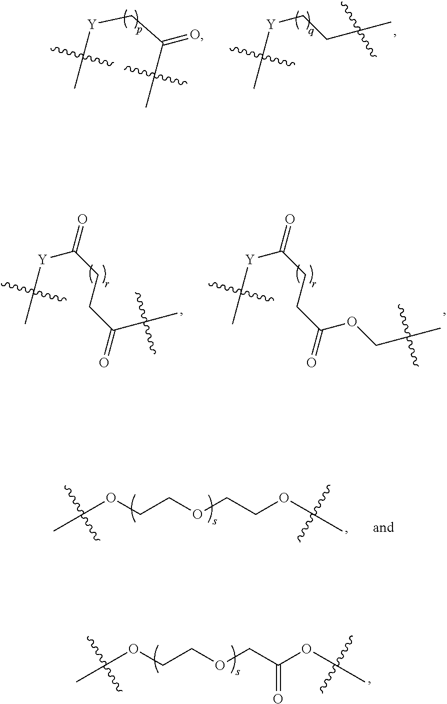

wherein Y is —NR^{f}—, —S—, —O— or —CR^{f1}R^{f2}—, wherein each of R^{f}, R^{f1} and R^{f2} is H or substituted or unsubstituted —C₁₋₆ alkyl; each of p, q, r and s is independently 0, 1, 2, 3 or 4; wherein, when L¹, L² or L³ is substituted, L¹, L² or L³ is substituted with one or more groups selected from Substituent Group A; or
(c) each of L¹, L² and L³, when present, is substituted or unsubstituted and is independently selected from:

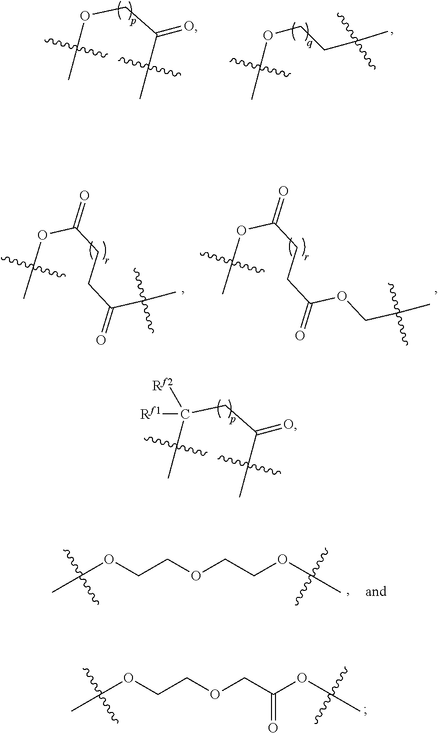

or
   (d) each of L¹, L² and L³, when present, is substituted or unsubstituted and is independently selected from:

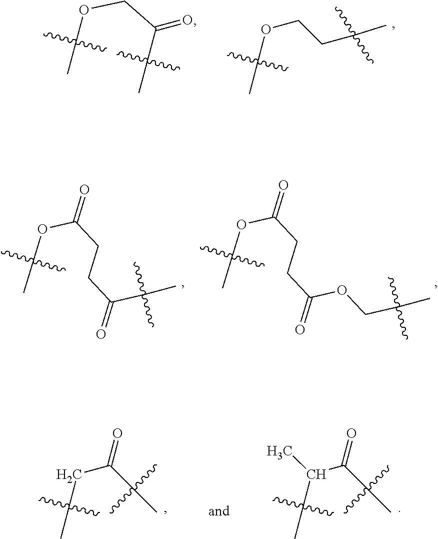

25. A method according to any one of items 21 to 24, wherein the compound of Formula (I) is a compound of Formula (Ia):

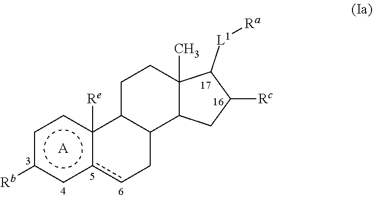

wherein:
R^{a} is selected from:

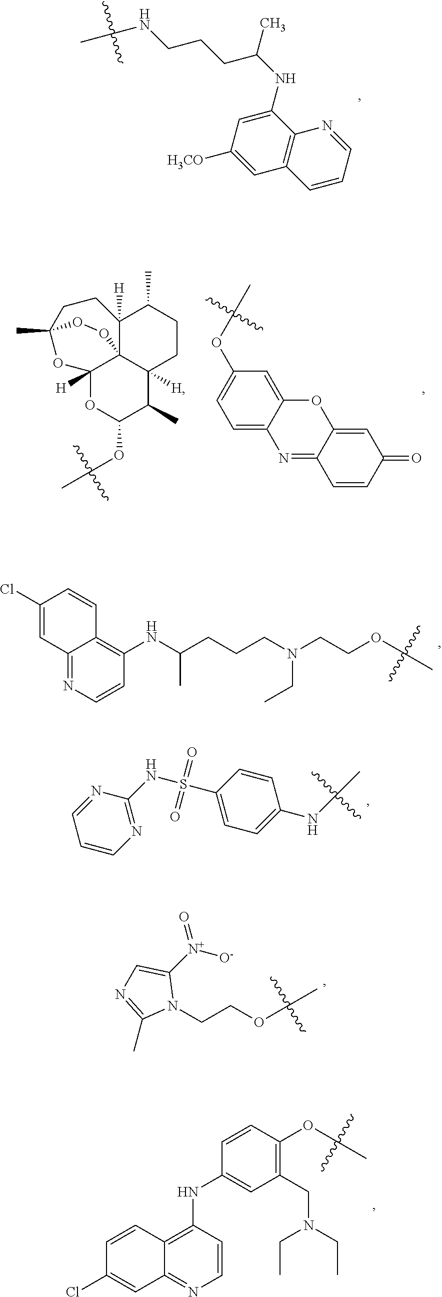

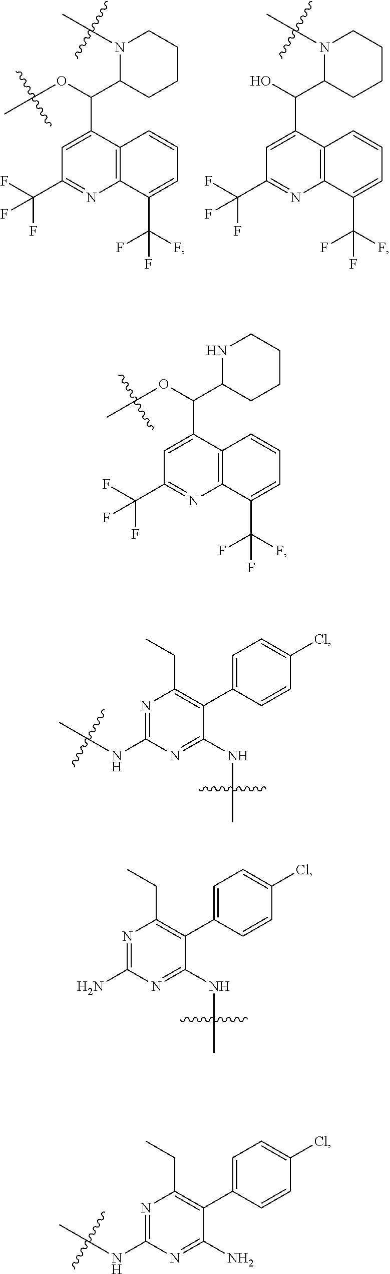

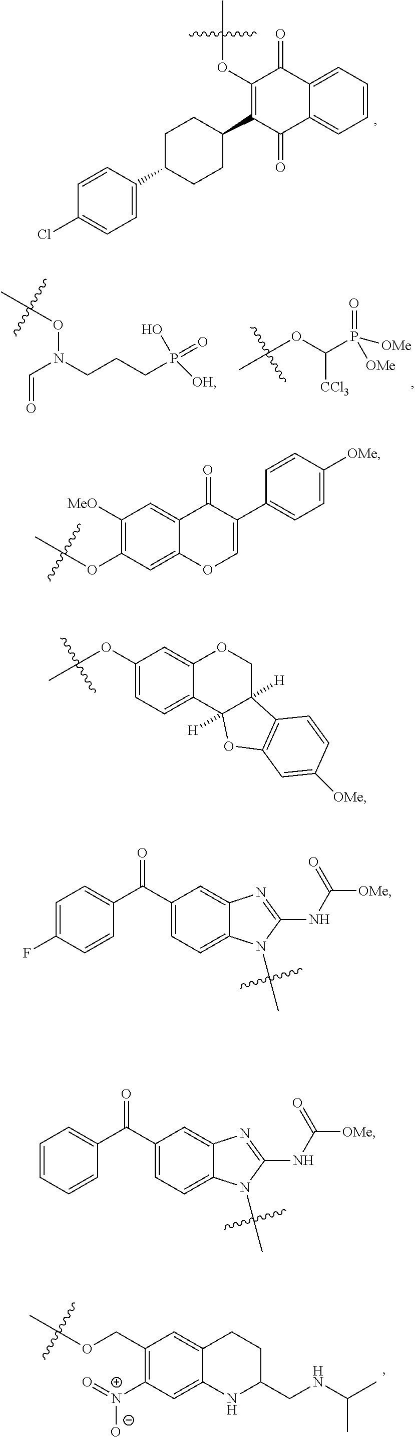

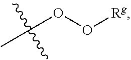

wherein R^{g} is H or substituted or unsubstituted —C₁₋₆ alkyl,

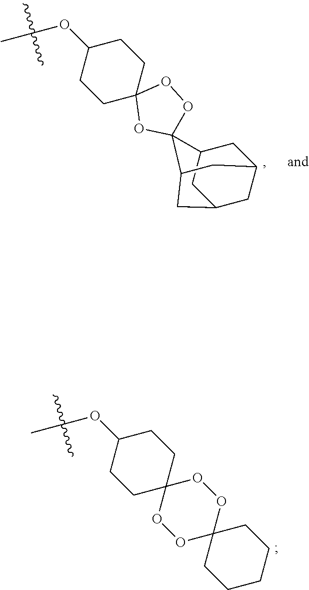 [SMILES: *OC1CCC2(CC1)OOC1(O2)C2CC3CC(C2)CC1C3 ; *OC1CCC2(CC1)OOC1(CCCCC1)OO2]

each of R^{b} and R^{c} is selected from —H, —OH, ═O, substituted or unsubstituted —C₁₋₁₀ alkyl, substituted or unsubstituted —C₂₋₁₀ alkenyl, substituted or unsubstituted —C₂₋₁₀ alkynyl, substituted or unsubstituted —C₃₋₁₀ cycloalkyl; substituted or unsubstituted —C₅₋₁₄ cycloalkenyl, or substituted or unsubstituted —C₆₋₁₄ cycloalkynyl, wherein, when R^{b} or R^{c} is substituted, R^{b} or R^{c} is substituted with one or more groups selected from Substituent Group A;
R^{e} is H or CH₃, or R^{e} is absent;
L¹ may be substituted or unsubstituted and is independently selected from:

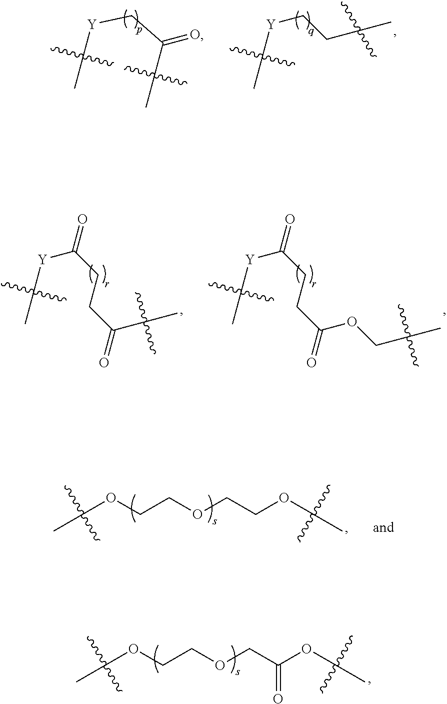

wherein Y is —NR^{f}—, —S—, —O— or —CR^{f1}R^{f2}—, wherein each of R^{f}, R^{f1} and R^{f2} is H or substituted or unsubstituted —C₁₋₆ alkyl; each of p, q, r and s is independently 0, 1, 2, 3 or 4; wherein, when L¹ is substituted, L¹ is substituted with one or more groups selected from Substituent Group A;
ring A may be a saturated or unsaturated ring, or ring A may be an aromatic ring, provided that when ring A is an aromatic ring, the C5-C6 bond is a single bond, R^{e} is absent, and R^{b} is not ═O;
[symbol] is a single bond or a double bond; provided that when the C4-C5 or C5-C10 bond is a double bond, the C5-C6 bond is a single bond; when the C5-C6 bond is a double bond, the C4-C5 and C5-C10 bonds are single bonds; and when the C3 forms a double bond with C2 or C4, R^{b} is not ═O;
or a stereoisomer thereof, or a pharmaceutically acceptable salt thereof.
26. A method according to any one of items 21 to 24, wherein the compound of Formula (I) is a compound of Formula (Ic):

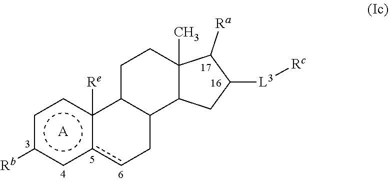

wherein:
each of R^{a} and R^{b} is selected from —H, —OH, ═O, substituted or unsubstituted —C₁₋₁₀ alkyl, substituted or unsubstituted —C₂₋₁₀ alkenyl, substituted or unsubstituted —C₂₋₁₀ alkynyl, substituted or unsubstituted —C₃₋₁₀ cycloalkyl; substituted or unsubstituted —C₅₋₁₄ cycloalkenyl, or substituted or unsubstituted —C₈₋₁₄ cycloalkynyl, wherein, when R^{a} or R^{b} is substituted. R^{a} or R^{b} is substituted with one or more groups selected from Substituent Group A;
   R^{c} is selected from:

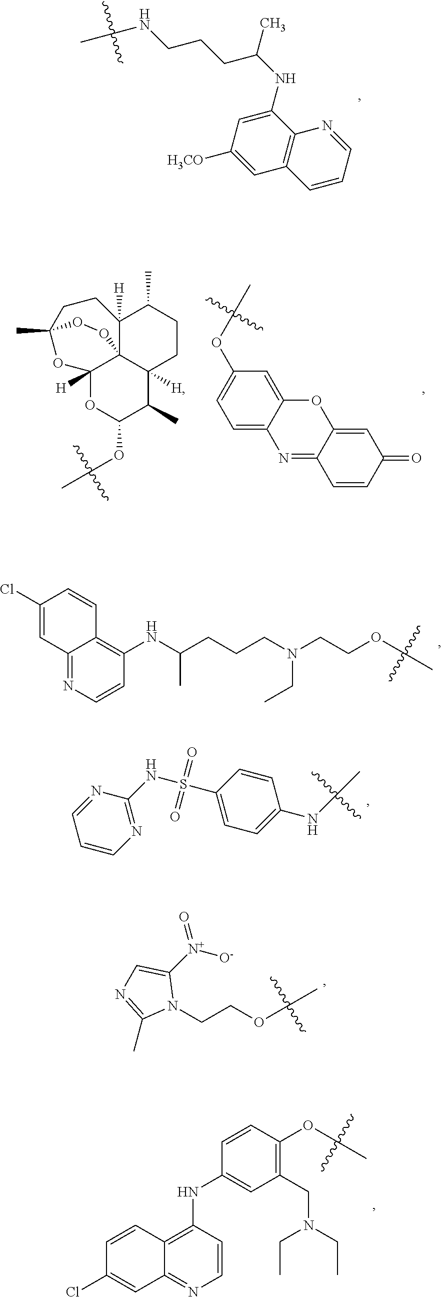

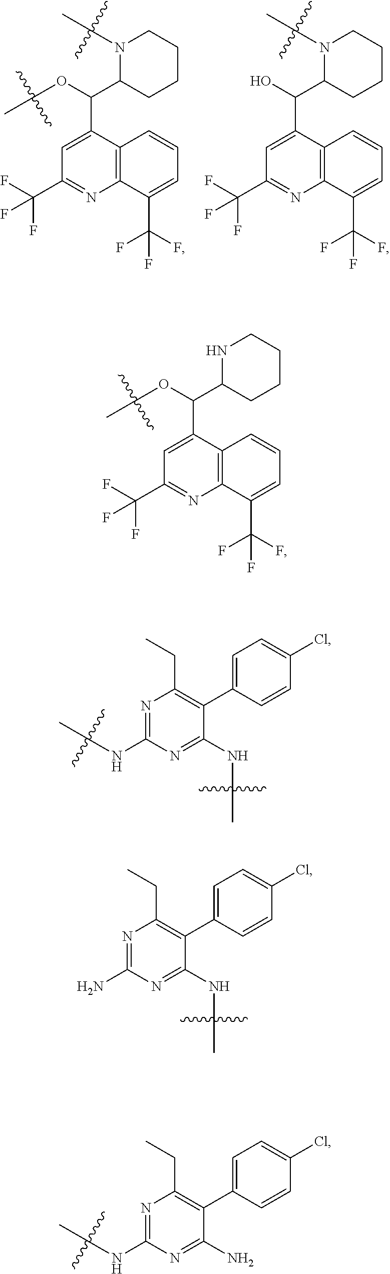

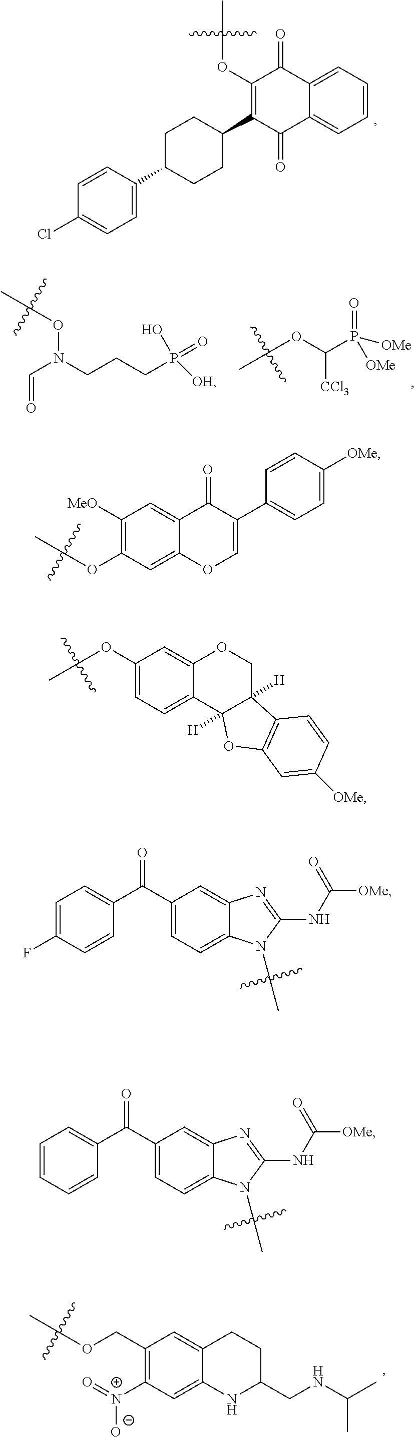

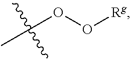

wherein R^{g} is H or substituted or unsubstituted —C₁₋₆ alkyl,

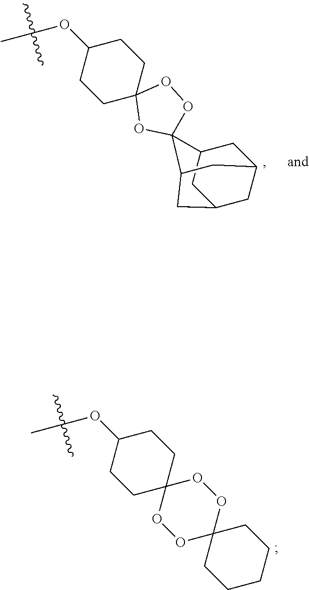 [SMILES: *OC1CCC2(CC1)OOC1(O2)C2CC3CC(C2)CC1C3 ; *OC1CCC2(CC1)OOC1(CCCCC1)OO2]

R^{e} is H or CH₃, or R^{e} is absent;
L³ may be substituted or unsubstituted and is independently selected from:

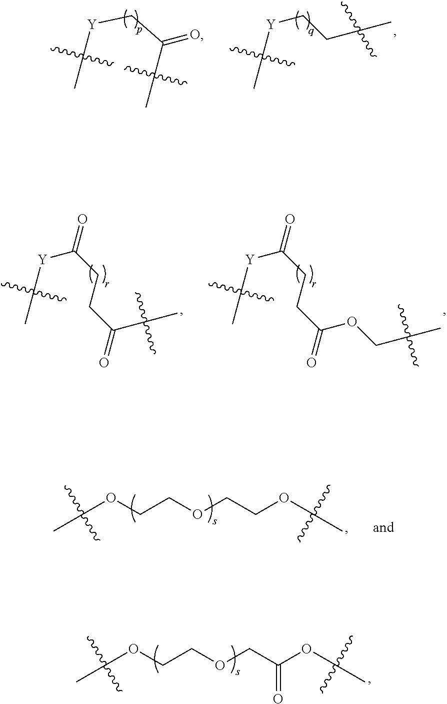

wherein Y is —NR^{f}—, —S—, —O— or —CR^{f1}R^{f2}—, wherein each of R^{f}, R^{f1} and R^{f2} is H or substituted or unsubstituted —C₁₋₆ alkyl; each of p, q, r and s is independently 0, 1, 2, 3 or 4; wherein, when L³ is substituted, L³ is substituted with one or more groups selected from Substituent Group A;
ring A may be a saturated or unsaturated ring, or ring A may be an aromatic ring, provided that when ring A is an aromatic ring, the C5-C6 bond is a single bond, R^{e} is absent, and R^{b} is not ═O;
[symbol] is a single bond or a double bond; provided that when the C4-C5 or C5-C10 bond is a double bond, the C5-C6 bond is a single bond; when the C5-C6 bond is a double bond, the C4-C5 and C5-C10 bonds are single bonds; and when the C3 forms a double bond with C2 or C4, R^{b} is not ═O;
or a stereoisomer thereof, or a pharmaceutically acceptable salt thereof.
27. A method according to item 25 or 26, wherein Formula (Ia) or Formula (Ic) comprises a steroid group selected from:

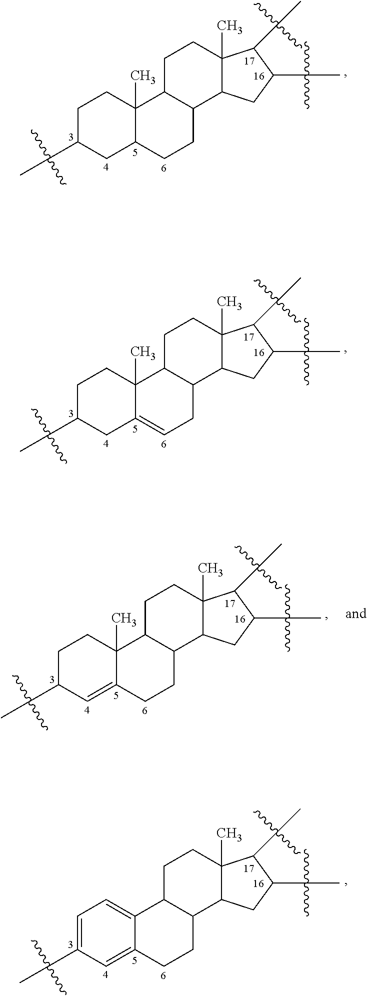 [SMILES: *C1CCC2(C)C(CCC3C2CCC2(C)C(*)C(*)CC32)C1 ; *C1CCC2(C)C(=CCC3C2CCC2(C)C(*)C(*)CC32)C1 ; *C1C=C2CCC3C(CCC4(C)C(*)C(*)CC34)C2(C)CC1 ; *c1ccc2c(c1)CCC1C2CCC2(C)C(*)C(*)CC12]

wherein the groups at C3, C16 and C17 may be attached in the α or β configuration on the respective ring.
28. A method according to any one of items 25 to 27, wherein each of L¹ and L³, when present, is substituted or unsubstituted and is independently selected from:

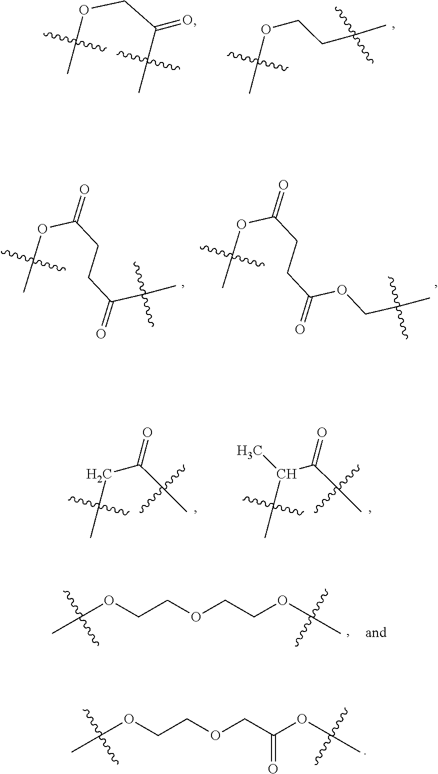

29. A method according to any one of items 25 to 28, wherein the anti-parasite moiety R^{a} or R^{c} is selected from:

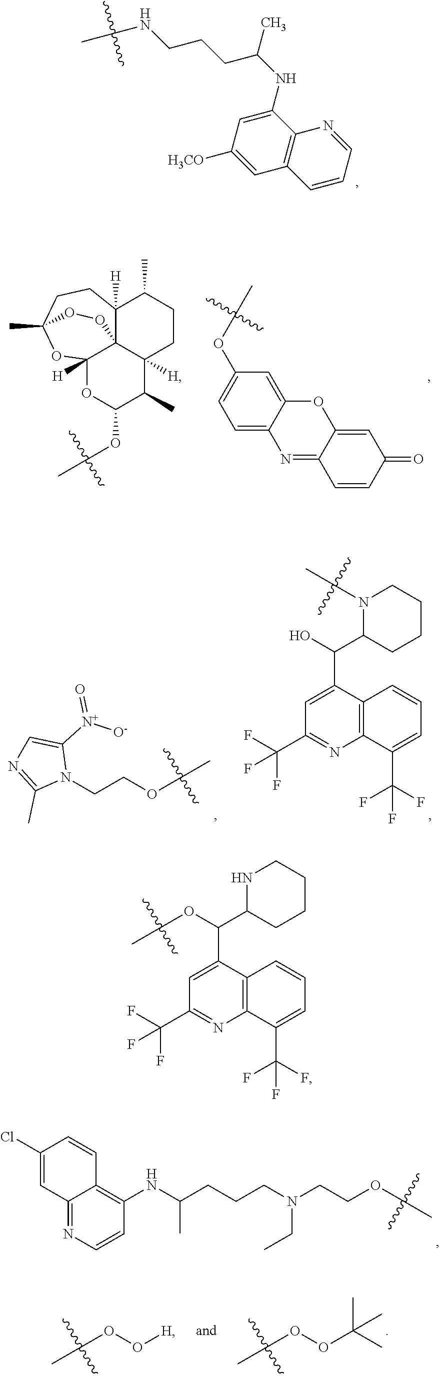

30. A method according to any one of items 21 to 29, wherein the parasite infection is a protozoan parasite infection or a helminth infection.
31. A method according to item 30, wherein the protozoan parasite infection is selected from a flagellate parasite infection, a ciliate parasite infection, an amoeba parasite infection, or an apicomplexan parasite infection.
32. A method according to item 30 or 31, where the protozoan parasite infection is caused by an organism selected from Giardia spp., Trichomonas spp., Leishmania spp., Trypanosoma spp., Balantidium coli, and Entamoeba histolytica.
33. A method according to item 31, wherein the apicomplexan parasite infection is caused by an organism selected from Plasmodium spp., Toxoplasma spp., Eimeria spp., Isospora spp., Theileria spp., Babesia spp., Sarcocystis spp., and Cryptosporidium spp.; or wherein the Leishmania parasite infection is caused by Leishmania tarentolae.
34. A method according to item 33, wherein:
   the apicomplexan parasite infection is caused by Plasmodium falciparum, Plasmodium vivax. Plasmodium ovale curtisi, Plasmodium ovale wallikeri, Plasmodium malariae, Plasmodium knowlesi or coccidia; or
   the apicomplexan parasite infection is caused by Eimeria spp. or Isospora spp.; or
   the apicomplexan parasite infection is caused by Toxoplasma gondii.
35. A compound of Formula (I) or a pharmaceutically acceptable salt thereof, for use in treating or preventing a parasite infection in a subject; wherein Formula (I) is:

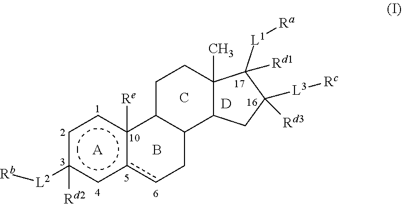

wherein:
each of R^{a} and R^{c} is independently selected from —H, —OH, ═O, substituted or unsubstituted —C₁₋₁₀ alkyl, substituted or unsubstituted —C₂₋₁₀ alkenyl, substituted or unsubstituted —C₂₋₁₀ alkynyl, substituted or unsubstituted —C₃₋₁₀ cycloalkyl, substituted or unsubstituted —C₅₋₁₄ cycloalkenyl, substituted or unsubstituted —C₈₋₁₄ cycloalkynyl, and an anti-parasite moiety; wherein at least one of R^{a} and R^{c} is an anti-parasite moiety:
R^{b} is selected from —H, —OH, ═O, substituted or unsubstituted —C₁₋₁₀ alkyl, substituted or unsubstituted —C₂₋₁₀ alkenyl, substituted or unsubstituted —C₂₋₁₀ alkynyl, substituted or unsubstituted —C₃₋₁₀ cycloalkyl, substituted or unsubstituted —C₅₋₁₄ cycloalkenyl, and substituted or unsubstituted —C₈₋₁₄ cycloalkynyl;
provided that: when R^{a} is —OH, L¹ is absent; when R^{a} is ═O, L¹ and R^{d1} are absent; when R^{b} is —OH, L² is absent; when R^{b} is ═O, L² is absent, R^{d2} is absent, and the C2-C3 bond and the C3-C4 bond are single bonds; when R^{c} is —OH, L³ is absent; when R^{c} is ═O, L³ and R^{d3} are absent;
when R^{a}, R^{b} or R^{c} is substituted, R^{a}, R^{b} or R^{c} is substituted with one or more groups selected from Substituent Group A;
R^{d1}, when present, is H or is a group that, together with R^{a}, forms a substituted or unsubstituted ozonide ring or a substituted or unsubstituted tetraoxane ring, wherein the optional substituent may be a —C₃₋₁₀ cycloalkyl; provided that when R^{d1} and R^{a} form a substituted or unsubstituted ozonide ring or a substituted or unsubstituted tetraoxane ring, L¹ is absent; wherein, when the ozonide or tetraoxane ring is substituted, the ozonide or tetraoxane ring is substituted with one or more —C₃₋₁₀ cycloalkyl groups;
R^{d2}, when present, is H; provided that when R^{d2} is present, the C2-C3 bond and the C3-C4 bond are single bonds;
R^{d3}, when present, is H or is a group that, together with R^{c}, forms a substituted or unsubstituted ozonide ring or a substituted or unsubstituted tetraoxane ring, wherein the optional substituent may be a —C₃₋₁₀ cycloalkyl; provided that when R^{d3} and R^{c} form a substituted or unsubstituted ozonide ring or a substituted or unsubstituted tetraoxane ring, L³ is absent; wherein, when the ozonide or tetraoxane ring is substituted, the ozonide or tetraoxane ring is substituted with one or more —C₃₋₁₀ cycloalkyl groups;
R^{e} is H or CH₃, or R^{e} is absent;
when present, L¹ is a group that provides a covalent linkage between R^{a} and the C17 of ring D;
when present, L² is a group that provides a covalent linkage between R^{b} and the C3 of ring A;
when present, L³ is a group that provides a covalent linkage between R^{c} and the C16 of ring D;
ring A may be a saturated or unsaturated ring, or ring A may be an aromatic ring, provided that when ring A is an aromatic ring, the C5-C6 bond is a single bond, R^{d2} is absent, R^{e} is absent, and R^{b} is not ═O;
[symbol] is a single bond or a double bond; provided that when the C4-C5 or C5-C10 bond is a double bond, the C5-C6 bond is a single bond; when the C5-C6 bond is a double bond, the C4-C5 and C5-C10 bonds are single bonds; and when the C3 forms a double bond with C2 or C4, R^{b} is not ═O;
or a stereoisomer thereof, or a pharmaceutically acceptable salt thereof.
36. Use of a compound of Formula (I) or a pharmaceutically acceptable salt thereof, in the manufacture of a medicament for the treatment or prevention of a parasite infection in a subject; wherein Formula (I) is:

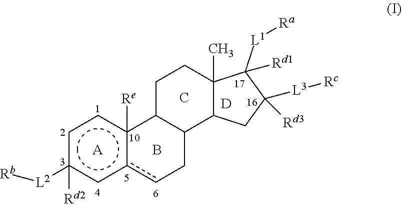

wherein:
each of R^{a} and R^{c} is independently selected from —H, —OH, ═O, substituted or unsubstituted —C₁₋₁₀ alkyl, substituted or unsubstituted —C₂₋₁₀ alkenyl, substituted or unsubstituted —C₂₋₁₀ alkynyl, substituted or unsubstituted —C₃₋₁₀ cycloalkyl, substituted or unsubstituted —C₅₋₁₄ cycloalkenyl, substituted or unsubstituted —C₈₋₁₄ cycloalkynyl, and an anti-parasite moiety; wherein at least one of R^{a} and R^{c} is an anti-parasite moiety;
R^{b} is selected from —H, —OH, ═O, substituted or unsubstituted —C₁₋₁₀ alkyl, substituted or unsubstituted —C₂₋₁₀ alkenyl, substituted or unsubstituted —C₂₋₁₀ alkynyl, substituted or unsubstituted —C₃₋₁₀ cycloalkyl, substituted or unsubstituted —C₅₋₁₄ cycloalkenyl, and substituted or unsubstituted —C₈₋₁₄ cycloalkynyl;
provided that: when R^{a} is —OH, L¹ is absent; when R^{a} is ═O, L¹ and R^{d1} are absent; when R^{b} is —OH, L² is absent; when R^{b} is ═O, L² is absent, R^{d2} is absent, and the C2-C3 bond and the C3-C4 bond are single bonds; when R^{c} is —OH, L³ is absent; when R^{c} is ═O, L³ and R^{d3} are absent;
when R^{a}, R^{b} or R^{c} is substituted, R^{a}, R^{b} or R^{c} is substituted with one or more groups selected from Substituent Group A;
R^{d1}, when present, is H or is a group that, together with R^{a}, forms a substituted or unsubstituted ozonide ring or a substituted or unsubstituted tetraoxane ring, wherein the optional substituent may be a —C₃₋₁₀ cycloalkyl; provided that when R^{d1} and R^{a} form a substituted or unsubstituted ozonide ring or a substituted or unsubstituted tetraoxane ring, L¹ is absent; wherein, when the ozonide or tetraoxane ring is substituted, the ozonide or tetraoxane ring is substituted with one or more —C₃₋₁₀ cycloalkyl groups;
R^{d2}, when present, is H; provided that when R^{d2} is present, the C2-C3 bond and the C3-C4 bond are single bonds;
R^{d3}, when present, is H or is a group that, together with R^{c}, forms a substituted or unsubstituted ozonide ring or a substituted or unsubstituted tetraoxane ring, wherein the optional substituent may be a —C₃₋₁₀ cycloalkyl; provided that when R^{d3} and R^{c} form a substituted or unsubstituted ozonide ring or a substituted or unsubstituted tetraoxane ring, L³ is absent; wherein, when the ozonide or tetraoxane ring is substituted, the ozonide or tetraoxane ring is substituted with one or more —C₃₋₁₀ cycloalkyl groups;
R^{e} is H or CH₃, or R^{e} is absent;
when present, L¹ is a group that provides a covalent linkage between R^{a} and the C17 of ring D;
when present, L² is a group that provides a covalent linkage between R^{b} and the C3 of ring A;
when present, L³ is a group that provides a covalent linkage between R^{c} and the C16 of ring D;
ring A may be a saturated or unsaturated ring, or ring A may be an aromatic ring, provided that when ring A is an aromatic ring, the C5-C6 bond is a single bond, R^{d2} is absent, R^{e} is absent, and R^{b} is not ═O;
[symbol] is a single bond or a double bond; provided that when the C4-C5 or C5-C10 bond is a double bond, the C5-C6 bond is a single bond; when the C5-C6 bond is a double bond, the C4-C5 and C5-C10 bonds are single bonds; and when the C3 forms a double bond with C2 or C4, R^{b} is not ═O;
or a stereoisomer thereof, or a pharmaceutically acceptable salt thereof.
37. A method of inhibiting the proliferation of a parasite, the method comprising contacting the parasite with an effective amount of a compound of Formula (I):

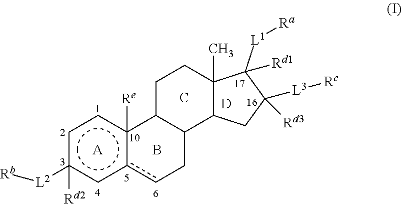

wherein:
each of R^{a} and R^{c} is independently selected from —H, —OH, ═O, substituted or unsubstituted —C₁₋₁₀ alkyl, substituted or unsubstituted —C₂₋₁₀ alkenyl, substituted or unsubstituted —C₂₋₁₀ alkynyl, substituted or unsubstituted —C₃₋₁₀ cycloalkyl, substituted or unsubstituted —C₅₋₁₄ cycloalkenyl, substituted or unsubstituted —C₈₋₁₄ cycloalkynyl, and an anti-parasite moiety; wherein at least one of R^{a} and R^{c} is an anti-parasite moiety:
R^{b} is selected from —H, —OH, ═O, substituted or unsubstituted —C₁₋₁₀ alkyl, substituted or unsubstituted —C₂₋₁₀ alkenyl, substituted or unsubstituted —C₂₋₁₀ alkynyl, substituted or unsubstituted —C₃₋₁₀ cycloalkyl, substituted or unsubstituted —C₅₋₁₄ cycloalkenyl, and substituted or unsubstituted —C₈₋₁₄ cycloalkynyl;
provided that: when R^{a} is —OH, L¹ is absent; when R^{a} is ═O, L¹ and R^{d1} are absent; when R^{b} is —OH, L² is absent; when R^{b} is ═O, L² is absent, R^{d2} is absent, and the C2-C3 bond and the C3-C4 bond are single bonds; when R^{c} is —OH, L³ is absent; when R^{c} is ═O, L³ and R^{d3} are absent;
when R^{a}, R^{b} or R^{c} is substituted, R^{a}, R^{b} or R^{c} is substituted with one or more groups selected from Substituent Group A;
R^{d1}, when present, is H or is a group that, together with R^{a}, forms a substituted or unsubstituted ozonide ring or a substituted or unsubstituted tetraoxane ring, wherein the optional substituent may be a —C₃₋₁₀ cycloalkyl; provided that when R^{d1} and R^{a} form a substituted or unsubstituted ozonide ring or a substituted or unsubstituted tetraoxane ring, L¹ is absent; wherein, when the ozonide or tetraoxane ring is substituted, the ozonide or tetraoxane ring is substituted with one or more —C₃₋₁₀ cycloalkyl groups;
R^{d2}, when present, is H; provided that when R^{d2} is present, the C2-C3 bond and the C3-C4 bond are single bonds;
R^{d3}, when present, is H or is a group that, together with R^{c}, forms a substituted or unsubstituted ozonide ring or a substituted or unsubstituted tetraoxane ring, wherein the optional substituent may be a —C₃₋₁₀ cycloalkyl; provided that when R^{d3} and R^{c} form a substituted or unsubstituted ozonide ring or a substituted or unsubstituted tetraoxane ring, L³ is absent; wherein, when the ozonide or tetraoxane ring is substituted, the ozonide or tetraoxane ring is substituted with one or more —C₃₋₁₀ cycloalkyl groups;
R^{e} is H or CH₃, or R^{e} is absent;
when present, L¹ is a group that provides a covalent linkage between R^{a} and the C17 of ring D;
when present, L² is a group that provides a covalent linkage between R^{b} and the C3 of ring A;
when present, L³ is a group that provides a covalent linkage between R^{c} and the C16 of ring D;
ring A may be a saturated or unsaturated ring, or ring A may be an aromatic ring, provided that when ring A is an aromatic ring, the C5-C6 bond is a single bond, R^{d2} is absent, R^{e} is absent, and R^{b} is not ═O;
[symbol] is a single bond or a double bond; provided that when the C4-C5 or C5-C10 bond is a double bond, the C5-C6 bond is a single bond; when the C5-C6 bond is a double bond, the C4-C5 and C5-C10 bonds are single bonds; and when the C3 forms a double bond with C2 or C4, R^{b} is not ═O;
or a stereoisomer thereof, or a salt thereof.
38. A method according to item 37, wherein the parasite is a protozoan parasite and the anti-parasite moiety is an anti-protozoan moiety, or the parasite is a helminth and the anti-parasite moiety is an anti-helminth moiety.
39. A method according to item 37, wherein the parasite is an apicomplexan parasite and the anti-parasite moiety is an anti-apicomplexan moiety.
40. A compound of Formula (I″):

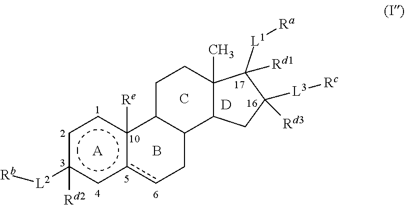

wherein:
each of R^{a} and R^{c} is independently selected from —H, —OH, ═O, substituted or unsubstituted —C₁₋₁₀ alkyl, substituted or unsubstituted —C₂₋₁₀ alkenyl, substituted or unsubstituted —C₂₋₁₀ alkynyl, substituted or unsubstituted —C₃₋₁₀ cycloalkyl, substituted or unsubstituted —C₅₋₁₄ cycloalkenyl, substituted or unsubstituted —C₈₋₁₄ cycloalkynyl, and an anti-parasite moiety; wherein at least one of R^{a} and R^{c} is an anti-parasite moiety;
R^{b} is selected from —H, —OH, ═O, substituted or unsubstituted —C₁₋₁₀ alkyl, substituted or unsubstituted —C₂₋₁₀ alkenyl, substituted or unsubstituted —C₂₋₁₀ alkynyl, substituted or unsubstituted —C₃₋₁₀ cycloalkyl, substituted or unsubstituted —C₅₋₁₄ cycloalkenyl, and substituted or unsubstituted —C₈₋₁₄ cycloalkynyl;
provided that: when R^{a} is —OH, L¹ is absent; when R^{a} is ═O, L¹ and R^{d1} are absent; when R^{b} is —OH, L² is absent; when R^{b} is ═O, L² is absent, R^{d2} is absent, and the C2-C3 bond and the C3-C4 bond are single bonds; when R^{c} is —OH, L³ is absent; when R^{c} is ═O, L³ and R^{d3} are absent;
when R^{a}, R^{b} or R^{c} is substituted, R^{a}, R^{b} or R^{c} is substituted with one or more groups selected from Substituent Group A;
R^{d1}, when present, is H or is a group that, together with R^{a}, forms a substituted or unsubstituted ozonide ring or a substituted or unsubstituted tetraoxane ring, wherein the optional substituent may be a —C₃₋₁₀ cycloalkyl; provided that when R^{d1} and R^{a} form a substituted or unsubstituted ozonide ring or a substituted or unsubstituted tetraoxane ring, L¹ is absent; wherein, when the ozonide or tetraoxane ring is substituted, the ozonide or tetraoxane ring is substituted with one or more —C₃₋₁₀ cycloalkyl groups;
R^{d2}, when present, is H; provided that when R^{d2} is present, the C2-C3 bond and the C3-C4 bond are single bonds;
R^{d3}, when present, is H or is a group that, together with R^{c}, forms a substituted or unsubstituted ozonide ring or a substituted or unsubstituted tetraoxane ring, wherein the optional substituent may be a —C₃₋₁₀ cycloalkyl; provided that when R^{d3} and R^{c} form a substituted or unsubstituted ozonide ring or a substituted or unsubstituted tetraoxane ring, L³ is absent; wherein, when the ozonide or tetraoxane ring is substituted, the ozonide or tetraoxane ring is substituted with one or more —C₃₋₁₀ cycloalkyl groups;
R^{e} is H or CH₃, or R^{e} is absent;
when present, L¹ is a group that provides a covalent linkage between R^{e} and the C17 of ring D;
when present, L² is a group that provides a covalent linkage between R^{b} and the C3 of ring A;
when present, L³ is a group that provides a covalent linkage between R^{c} and the C16 of ring D;
ring A may be a saturated or unsaturated ring, or ring A may be an aromatic ring, provided that when ring A is an aromatic ring, the C5-C6 bond is a single bond, R^{d2} is absent, R^{e} is absent, and R^{b} is not ═O;
[symbol] is a single bond or a double bond; provided that when the C4-C5 or C5-C10 bond is a double bond, the C5-C6 bond is a single bond; when the C5-C6 bond is a double bond, the C4-C5 and C5-C10 bonds are single bonds; and when the C3 forms a double bond with C2 or C4, R^{b} is not ═O;
or a stereoisomer thereof, or a salt thereof;
wherein the anti-parasite moiety is selected from:

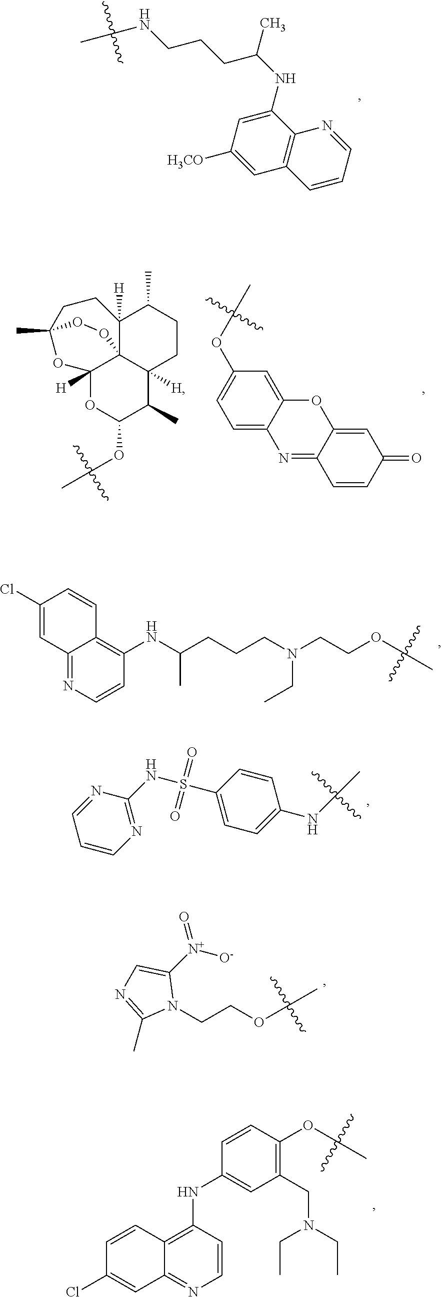

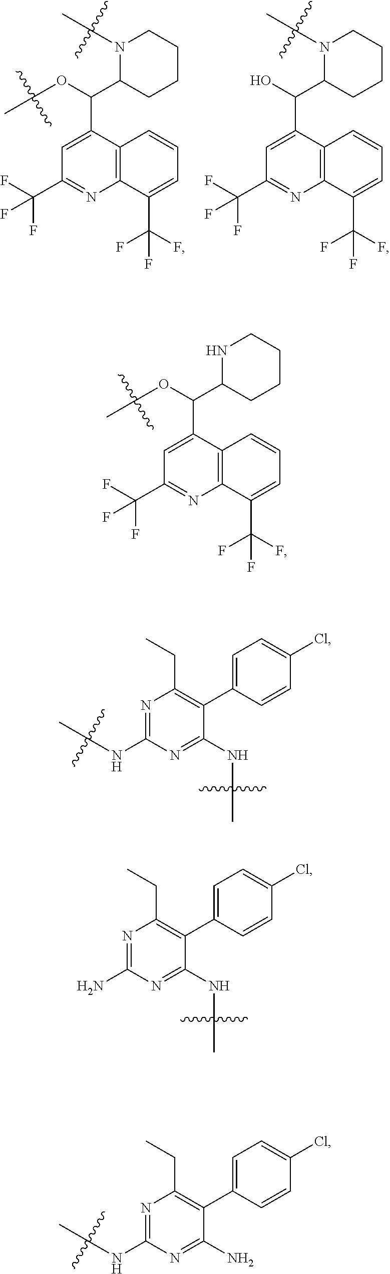

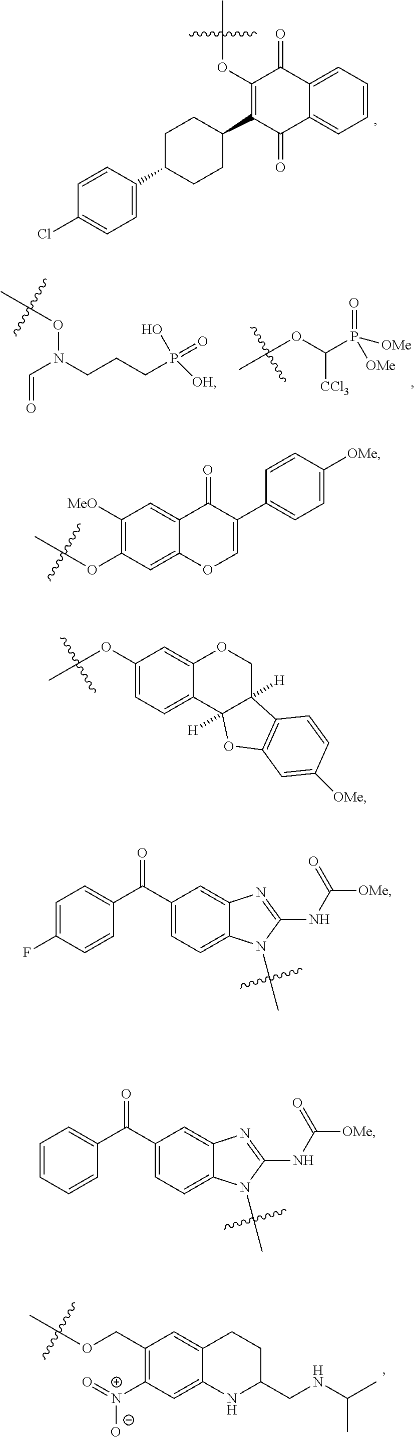

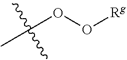

wherein R^{g} is H or substituted or unsubstituted —C₁₋₆ alkyl,

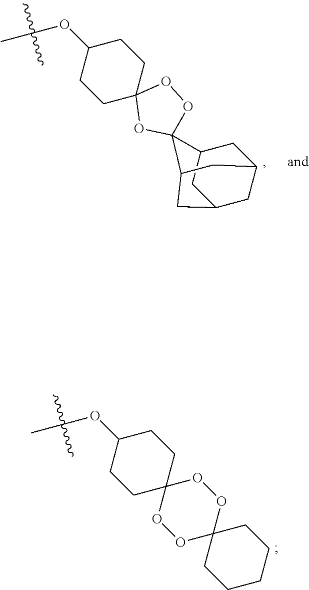 [SMILES: *OC1CCC2(CC1)OOC1(O2)C2CC3CC(C2)CC1C3 ; *OC1CCC2(CC1)OOC1(CCCCC1)OO2]

and
   wherein each of L¹, L² and L³, when present, is substituted or unsubstituted and is independently selected from:

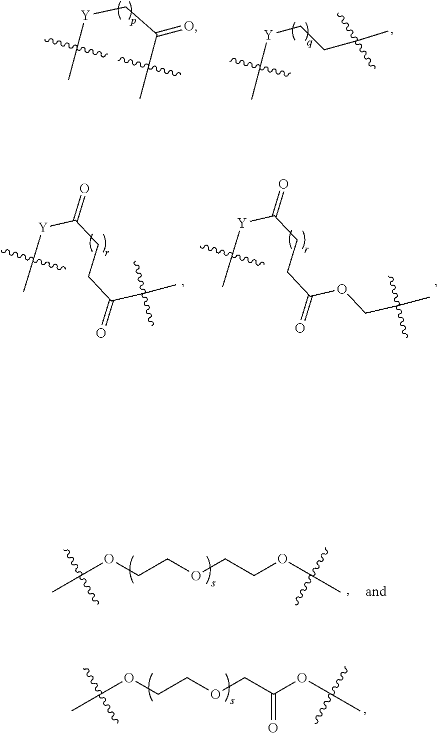

wherein Y is —NR^{f}—, —S—, —O— or —CR^{f1}R^{f2}—, wherein each of R^{f}, R^{f1} and R^{f2} is H or substituted or unsubstituted —C₁₋₆ alkyl; each of p, q, r and s is independently 0, 1, 2, 3 or 4; wherein, when L¹, L² or L³ is substituted, L¹, L² or L³ is substituted with one or more groups selected from Substituent Group A.
41. A compound according to item 40, wherein Formula (I″) is selected from:

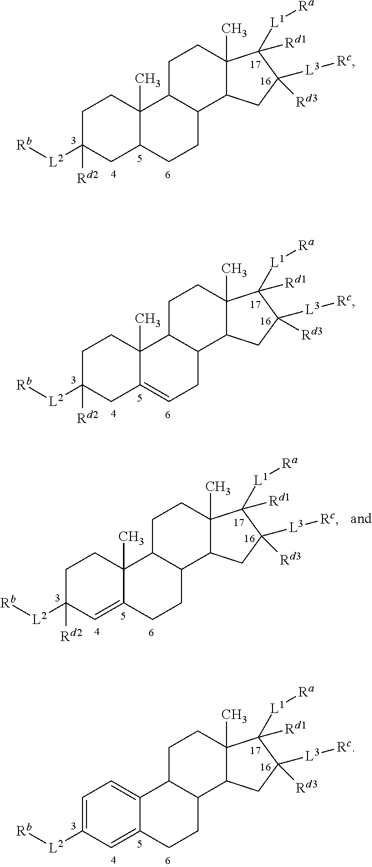

42. A compound according to item 40 or 41, wherein Formula (I″) is selected from:

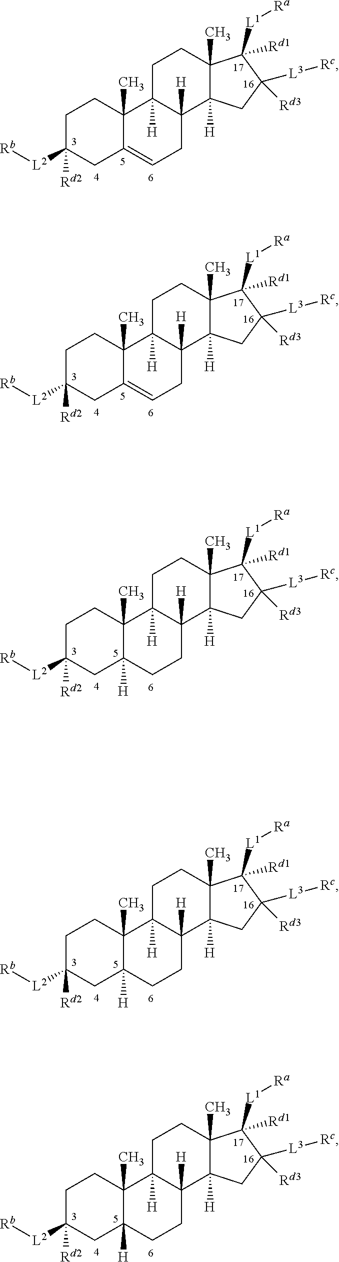

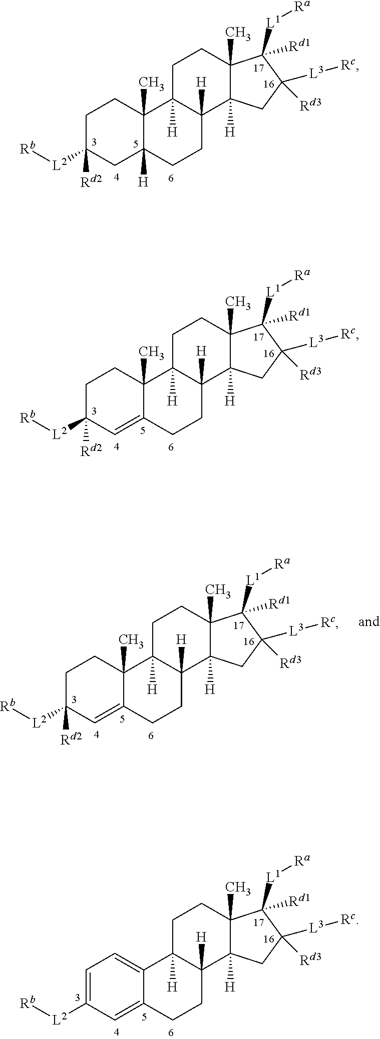

43. A compound according to any one of items 40 to 42, wherein each of L¹, L² and L³, when present, is substituted or unsubstituted and is independently selected from:

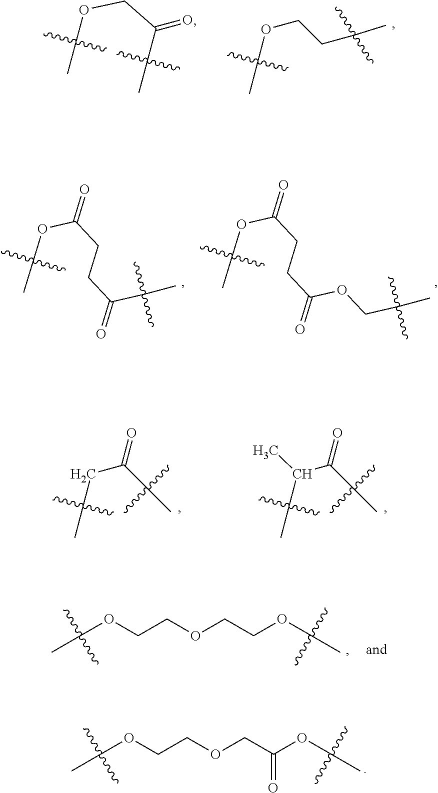

44. A compound according to item 40 selected from:

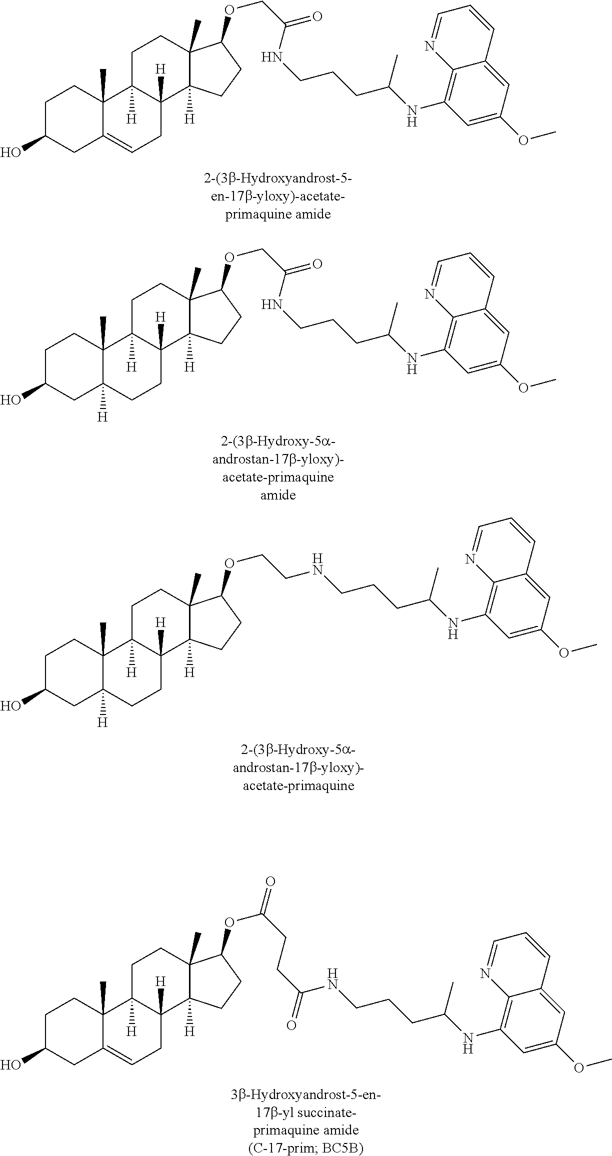

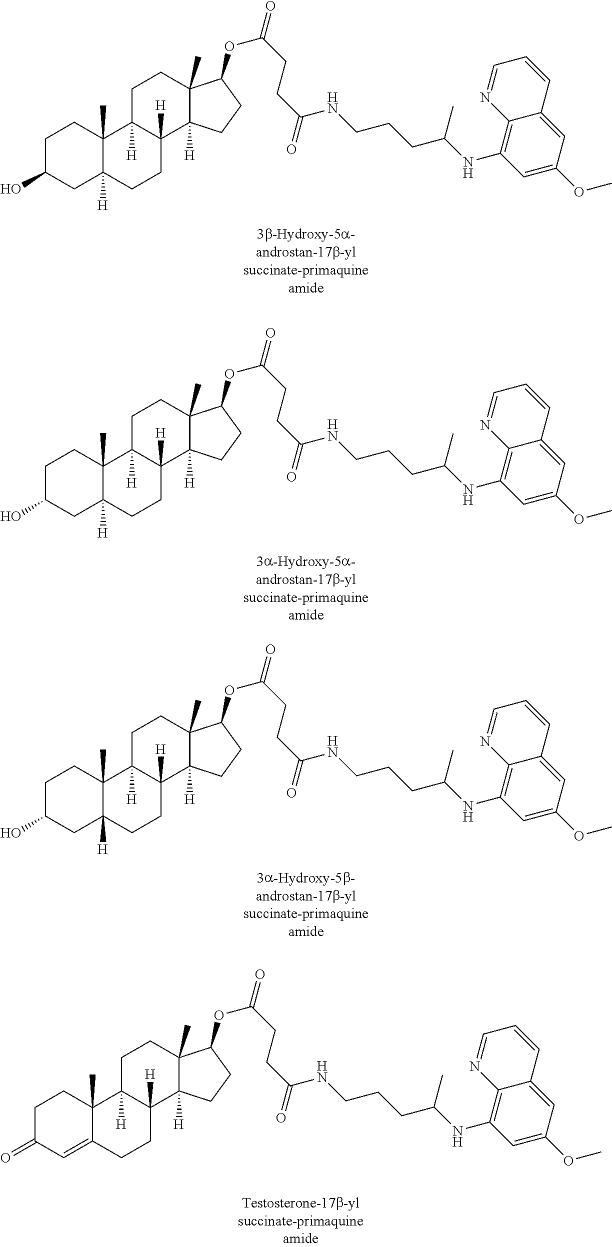

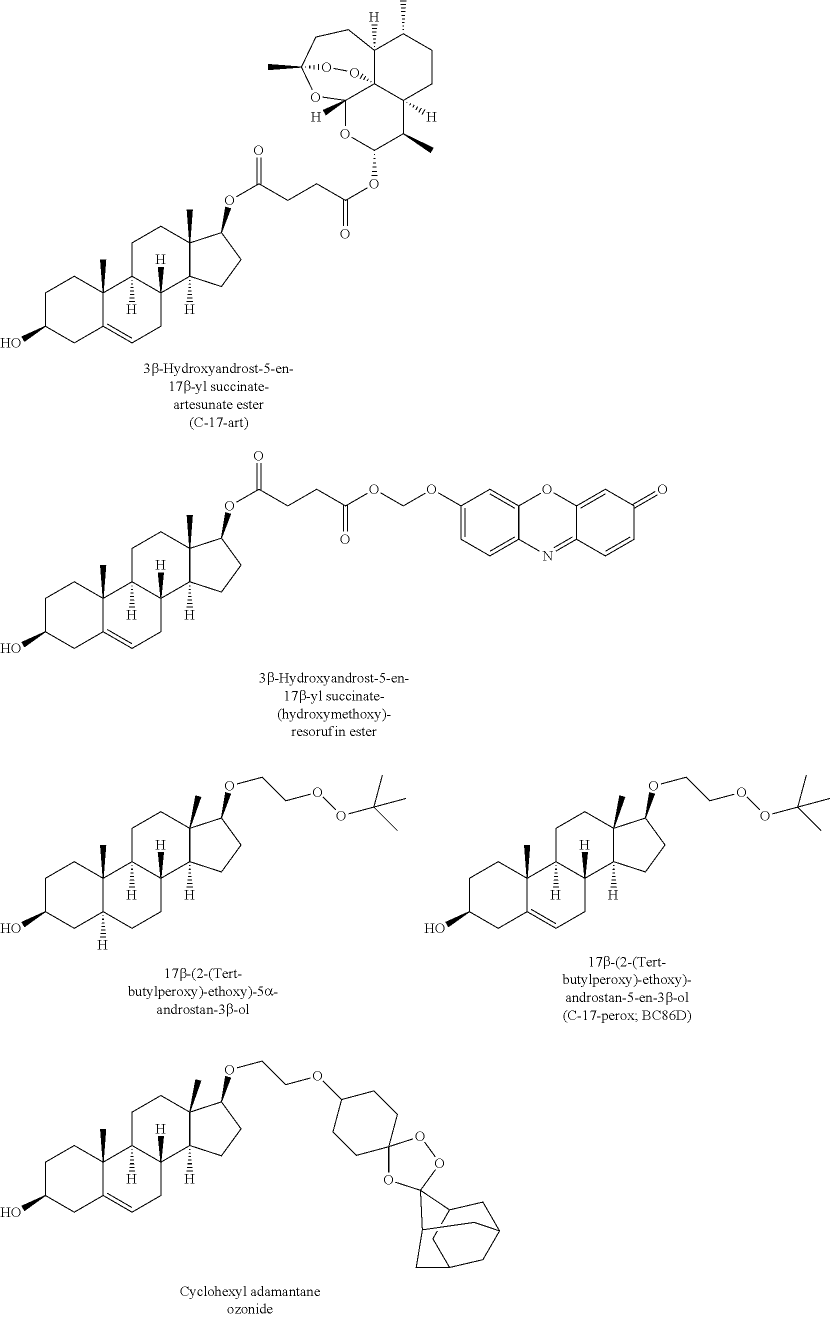

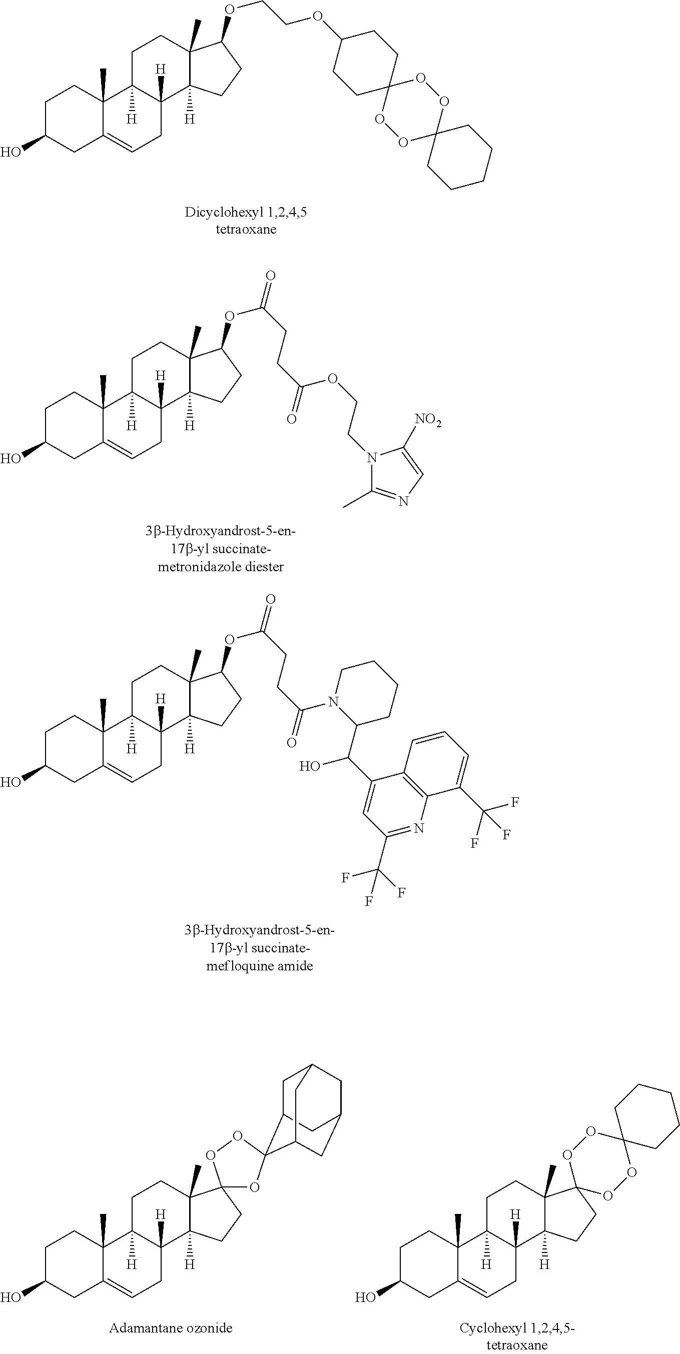

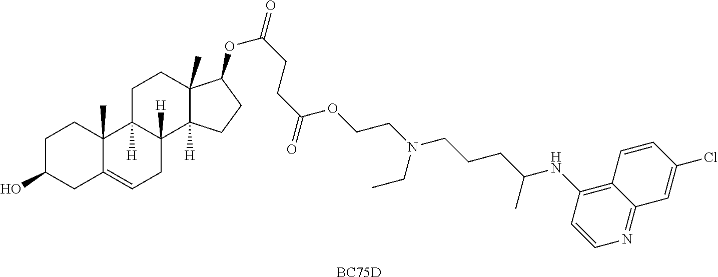

or a salt thereof.
45. A composition comprising a compound of Formula (I″) according to any one of items 40 to 44, or a salt thereof, and a suitable carrier, adjuvant or diluent.
46. A pharmaceutical composition comprising a compound of Formula (I″) according to any one of items 40 to 44, or a pharmaceutically acceptable salt thereof, and a pharmaceutically acceptable carrier, adjuvant or diluent.

### BRIEF DESCRIPTION OF THE FIGURES

Embodiments of the present invention will be further described, by way of example only, with reference to the accompanying drawings, in which:

FIG. 1 is a diagram showing the life-cycle of the malaria parasite Plasmodium falciparum (from Maier et al., Trends in Parasitology 2019).

FIG. 2 is a table showing the content (nmol/10⁹ cells, Mean±SD) and percentage (in parentheses) of cholesterol of the blood-stage P. falciparum infected red blood cells (Roman numerals indicate different development stages of the sexual parasite stages (gametocytes). Parasites were magnetically enriched to higher than 95% parasitaemia (% infected red blood cells/total red blood cells) and global lipidomics of whole-cell extracts were analysed by mass spectrometry, uRBC, uninfected red blood cell; I-V, gametocytes stage I to V; n.d., not detected. (percentage decreases during development, since total lipid amounts increase upon infection and parasite development) (modified from Tran et al., 2016 Malaria Journal, 15. Article number: 73). The total amount of cholesterol present in infected RBCs increases as the parasite develops.

FIG. 3 is fluorescence imaging of uninfected RBCs (a), trophozoite infected RBCs (b) stained with 25-NBD cholesterol (a cholesterol analogue). Imaging was performed on a DeltaVision Elite microscope (Applied Precision) at the same fluorescent recording setting, uRBC uninfected red blood cells. BF bright field. Note: 25-NBD cholesterol crosses membranes and hence also stains the parasite. Scale bar: 5 μm (from Tran et al., 2016 Malaria Journal). The figures shows that the RBC plasma membrane is depleted of cholesterol as the parasite develops and accumulates cholesterol.

FIG. 4 are images of uninfected RBC and P. falciparum parasite infected red blood cells stained with a fluorescent probe, that binds to cholesterol (red) and DNA stain (blue). As the parasite develops inside the red blood cell (maturation of the asexual-stages of P. falciparum), less cholesterol can be detected on the red blood cell plasma membrane (note: probe does not cross membranes, hence only cholesterol exposed on the outside of the membrane is detected).

FIG. 5A are graphs showing the effect of cholesterol depletion from RBC and media on P. falciparum proliferation over time. The concentration dependent effect of cholesterol depletion from RBCs on asexual growth of P. falciparum over 30 mins (a). 24 hrs (b) and 72 hrs (c), incubated in cholesterol-maintained and cholesterol-depleted conditions. Error bars represents mean±SD of three independent biological replicates.

FIG. 5B is a graph showing the effect of cholesterol depletion on cell volume of uninfected and infected RBC. Uninfected and infected red blood cells were incubated at different concentrations of MBCD, which depletes cholesterol from membranes. After 6 hours at the highest MBCD concentration the volume of infected RBCs increases significantly, whereas the same concentration has no effect on uninfected RBCs. This points towards an increased role of cholesterol in the infected RBCS. Depletion of cholesterol is detrimental to P. falciparum growth. ***p<0.001

FIG. 6 is a graph and image showing the uptake of 22-NBD-cholesterol in infected and uninfected RBCs. Uninfected (uRBC) and infected (iRBC) red blood cells were incubated with the cholesterol analogue 22-NBD-cholesterol for 24 hrs. A conjugate consisting of steroid and fluorophore is taken up by the parasite. Uptake in iRBC is increased ˜8-fold compared to uRBC. Accumulation of the cholesterol analogue in the parasite can be seen.

FIG. 7 are graphs and images showing the uptake of various fluorescent cholesterol derivatives modified at C3 or C17. The C17 and C3 of cholesterol was modified by the addition of different fluorophores (except dehydroergosterol, which exhibits intrinsic fluorescence). The second column shows the structure. Uptake into infected and uninfected red blood cells after 24 hrs was quantified by measuring changes in fluorescence (third column) and visualised using a Deltavision Deconvolution microscope (fourth/fifth column). Steroid conjugates/cholesterol analogues in green and parasite DNA in blue; except dehydroergosterol, where parasite mitochondria are also in red. Conjugates at the sidechain are more effectively taken up than conjugates at the C3.

FIG. 8A/B are dose response curves of primaquine alone, primaquine coupled to a steroid via a linker, primaquine coupled to the linker, and the steroid coupled to the linker. The graphs show the effect the steroid-linked primaquine compound has on asexual P. falciparum growth compared to primaquine alone. Control compounds (primaquine & linker, or steroid & linker) were used to show specificity. FIG. 8B shows that primaquine conjugated to a steroid is more effective against P. falciparum asexual intra-erythrocytic stages than primaquine alone.

FIG. 8C is a graph showing time dependency of dose response curves of primaquine alone and primaquine coupled to steroids against asexual P. falciparum stages. The IC50 is for primaquine and BC5B is shown on the right.

FIG. 8D is a graph showing an alternative representation of the data shown in FIG. 8C showing determined IC50 values of 3 biological replicates. **p<0.01; *p<0.05; NS non-significant. FIGS. 8C/D show that conjugated primaquine impacts growth of P. falciparum asexual intra-erythrocytic stages faster than primaquine alone.

FIG. 8E is a graph showing time dependency of dose-response curves of control compounds BC9B and HJB8a53 (primaquine & linker, or steroid & linker) against asexual P. falciparum stages. Error bars indicate SD of three biological replicates. IC50 for BC9B and HJB8a53 is shown on the right.

FIG. 8F is a graph showing time dependency of dose-response curves of the commercially available anti-malarial chloroquine against asexual P. falciparum stages. Error bars indicate SD of three biological replicates. IC50 is shown on the right.

FIG. 9A is a graph showing the effect of primaquine, steroid-coupled primaquine (BCSB), BC9B, HJB8a53, complete culture medium (CCM) and DMSO on P. falciparum gametocytes over 72 hrs (stage III-IV). All tested compounds were used at a concentration of 10 μM. Complete culture media (CCM) only or CCM/0.1% DMSO were used as controls. Error bars indicate SD of 2 technical replicates. Conjugated primaquine increases efficacy against P. falciparum gametocyte stages.

FIG. 9B is a graph showing the effect of steroid-coupled primaquine, primaquine (BC5B), BC9B and HJB8a53 on P. falciparum gametocytes over 72 hrs (stage III-IV). Steroid-coupled primaquine seems to be 6-times more effective against late-stage gametocytes than primaquine alone. However, the curve becomes flatter; (n=1)—in technical duplicates.

FIG. 9C are graphs showing sex-specific effect of different compounds against early (I-III) stage (A) and late stage (III-IV) P. falciparum gametocytes. The data indicates that both early and late-stage gametocytes are equally susceptible to the BC5B. Both gametocyte sexes can be targeted equally with the coupled compounds. Sex-specificity (if desired) might be achieved by using cargo-drugs, that acts specifically on one sex only.

FIG. 10A is a graph and images showing a steroid-resorufin conjugate is cleaved within the cell, indicating that the probe is taken up and metabolised. Uptake of the probe is approximately 2-fold higher in infected RBCs (iRBCs) than uninfected RBCs (uRBCs). Resorufin fluorescence (red) is observed within the parasite (DNA stained blue).

FIG. 10B is a dose-response curve comparing resorufin and Payb193 at 72 hrs. The steroid-resorufin probe can kill the parasite at lower concentrations than the uncoupled resorufin base molecule, potentially indicating that the higher uptake into iRBCs can be employed as a specific drug delivery system. Steroid conjugated resorufin is taken up by the parasite and cleaved.

FIG. 11A is a graph showing the inhibitory effect of steroid-coupled primaquine on the growth of Toxoplasma gondii over 72 hrs compared to that of primaquine, BC9B and HJB8a53. The IC₅₀ values of steroid-coupled primaquine (BC5B), primaquine, primaquine & linker (BC9B), steroid & linker (HJB8a53) were determined via a dose-response curve. Error bars indicate SD of three biological replicates.

FIG. 11B is a graph showing an alternative representation of data shown in FIG. 11A showing determined IC50 values of 4 biological replicates. Primaquine conjugated to a steroid is more effective against T. gondii tachyzoite stages than primaquine alone. **p<0.01; NS non-significant

FIG. 11C is a graph showing the inhibitory effect of steroid-coupled primaquine on the growth of human foreskin fibroblasts (HFF) (the host cells of Toxoplasma gondii) compared to that of primaquine, BC9B and HJB8a53 over 72 hrs. The IC₅₀ values of steroid-coupled primaquine (BC5B), primaquine, primaquine & linker (BC9B), steroid & linker (HJB8a53) were determined via a dose-response curve. Steroid-conjugated primaquine is less toxic to human foreskin fibroblast cells than primaquine alone. Error bars indicate SD of three biological replicates.

FIG. 11D is a graph showing the inhibitory effect of cholesterol and DHEA on the growth of asexual stages of P. falciparum. Adding additional cholesterol in different solvents to the culture medium does not affect the growth of P. falciparum asexual stages at relevant concentrations. Error bars indicate SD of three biological replicates.

FIG. 12A shows the structure of BC41B and FIG. 12B is a graph showing the effect of peroxide coupled steroid (BC41B) on the proliferation of asexual P. falciparum parasites over 72 hrs. Steroids can be modified to act as anti-Plasmodium drugs. Error bars indicate SD of three biological replicates.

FIG. 13A is a Table showing the structure of artesunate, artesunate conjugate to a steroid on C17 (PAYb076a), artesunate conjugate to cholesterol on C3 (GGA4), and steroid (HJB8a53, GGA3) and artesunate (GGA5) controls.

FIG. 13B is a dose-response curve showing the effect of differently coupled steroid-artesunate conjugates on the proliferation of asexual P. falciparum parasites over 72 hrs. Error bars indicate SD of three biological replicates. Coupling via C17 of the steroid (PAYb076a) results in better activity than coupling via C3 (GGA4).

FIG. 13C is a dose-response curve showing the effects of differently coupled steroid-artesunate conjugates on the proliferation of asexual P. falciparum parasites over 72 hrs. The graph shows the same data as FIG. 13B with the addition of other control compounds. C17-conjugated artesunate is more effective than C3-conjugated artesunate. Error bars indicate SD of three biological replicates.

FIG. 14 shows quantification of NBD-lipid fluorescence, measured by foldchange of NBD mean fluorescence intensity (MFI) of whole cells in flow cytometry ((i) 22-NBD-cholesterol, and (ii) 3-hexanoyl-NBD-cholesterol). Shown are mean values (±S.D.) n=3 independent experiments. NS not significant; ***p<0.001 (ANOVA).

FIG. 14A-14C: Coupling of primaquine to a steroid increases its potency against P. falciparum. (A) Chemical structures of (I) primaquine; (ii) steroid with primaquine conjugated from C-17 (C-17-prim; BCSB), (ill) cholesterol with primaquine conjugated from C-3 (C-3-prim; BC64C) (lv) primaquine with a linker (prim-link; BC9B), (v) steroid molecule with a linker at C-17 (C-17-link; HJB8a53), and (vi) cholesterol with a linker at C-3 (C-3-link; GGA3). (B) Dose response assay (24 hours) of compounds against asexual P. falciparum growth (starting at ring stages). Shown are mean values (±S.D.). 50% inhibitory concentration (IC₅₀) is in brackets. n=3 independent experiments. (C) Dose response assay (48 hours) against sexual (stage III-IV gametocytes) P. falciparum viability. Shown are mean values (±S.D.). IC₅₀ is in brackets. n=4 independent experiments.

FIG. 15: Coupling of primaquine to a steroid increases its potency against Apicomplexan parasites. (A) Comparison of 50% inhibitory concentrations (IC₅₀) values against asexual P. falciparum from FIG. 15B. Individual data points are the IC₅₀ values from independent experiments; centre bar is the mean value (±S.D.) of these data points. (B—C) Dose response curves of (B) prim, C-17-prim, and (C) prim-link. C-17-link, C-3-prim, C-3-link, against asexual P. falciparum growth at 24, 48 and 72 hours. Shown are mean values (±S.D.). IC₅₀ is in brackets. (D) Comparison of IC₅₀ values against sexual P. falciparum gametocytes from FIG. 14C. Individual data points are the IC₅₀ values from independent experiments; centre bar is the mean value (±S.D.) of these data points. n=4 independent experiments. (E) Comparison of IC₅₀ values against P. berghei liver stages from FIG. 16A. Individual data points are the IC₅₀ values from independent experiments; centre bar is the mean value (±S.D.) of these data points. n=3 independent experiments. (F) Comparison of IC₅₀ values against Toxoplasma gondii from FIG. 16E. Individual data points are the IC₅₀ values from independent experiments; centre bar is the mean value (±S.D.) of these data points. n=4 independent experiments. NS not significant; *p<0.05; **p<0.01 (ANOVA).

FIG. 16: Steroid conjugation enhances the inhibitory effect of primaquine against P. berghei liver stages and Toxoplasma gondii tachyzoites, and has a lower inhibitory effect than primaquine on the viability of human hepatoma cells and fibroblasts (A) Dose response assay (48 hours) against P. berghei growth incubated with primaquine, C-17-prim, prim-link, or C-17-link. Shown are mean values (±S.D.) IC₅₀ is in brackets. n=3 independent experiments. (B) P. berghei liver stage schizonts expressing GFP in human hepatoma cells (Huh7) (48 hours post-infection) treated with 0.3 or 3 μM primaquine or C-17-prim, or solvent control (DMSO), visualised by fluorescence microscopy. GFP fluorescence (showing the parasite, depicted in green) was detected at 470 nm (ex)/525 nm (em) and DAPI fluorescence (DNA; depicted in blue) was detected at 359 nm (ex)/445 nm (em). Scale bar=10 μm. Fluorescence intensity is not comparable between images. (C) Normalised mean size quantification (n=2 independent experiments; mean of all parasites (0-500) per coverslip) and (D) number of parasites per coverslip (n=3 independent experiments) of liver stage parasites from (B) after inoculation with an equal sporozoite number and treatment with 0.3 or 3 μM primaquine or C-17-prim, or solvent control (DMSO). Shown are individual data points for each technical replicate, with centre bar representing mean values (±S.D.). NS not significant; **p<0.01; ***p<0.001 (ANOVA). (E) Dose response assay (72 hours) against Toxoplasma gondii tachyzoite growth incubated with primaquine, C-17-prim, prim-link, or C-17-link. Shown are mean values (±S.D.). IC₅₀ is in brackets. n=4 independent experiments. (F) Dose response assay (48 hours) against human hepatoma cell (Huh7) viability incubated with primaquine, C-17-prim, prim-link, or C-17-link. Shown are mean values (±S.D.). Calculated IC₅₀ is in brackets. (G) Dose response assay (72 hours) against human foreskin fibroblasts (HFF) growth incubated with primaquine, C-17-prim, prim-link, or C-17-link. Shown are mean values (±S.D.). IC₅₀ is in brackets. n=3 independent experiments. (H) Dose response assay (96 hours) against human embryonic kidney cells (HEK293) growth incubated with primaquine, C-17-prim, prim-link, or C-17-link. Shown are mean values (±S.D.). IC₅₀ is in brackets. n=3 independent experiments.

FIG. 17: Steroid coupling of artesunate increases its potency against resistant P. falciparum. (A) Chemical structures of (i) artesunate; (ii) steroid with artesunate conjugated from C-17 (C-17-art), (iii) artesunate with a linker (art-link), and (iv) steroid molecule with a linker at C-17 (C-17-link). (B) Quantification of ring stage survival using DNA replication. Shown are mean values (±S.D.). NS not significant; **p<0.01; ***p<0.001 (ANOVA). n=2 independent experiments.

FIG. 18: Steroid-coupled peroxides inhibit P. falciparum growth (A) Structure of (i) DHEA, (ii) DHEA-derived peroxide (C-17-perox), and (iii) di-tert-butyl peroxide. (B) Dose response curve (72 hours) of compounds against asexual P. falciparum growth. Shown are mean values (±S.D.). IC₅₀ is in brackets. n=3 independent experiments. (C) Dose response curve (96 hours) against human embryonic kidney (HEK293) cell viability incubated with DHEA, C-17-perox, and di-tert-butyl peroxide. Shown are mean values (±S.D.). IC₅₀ is in brackets. n=4 independent experiments.

FIG. 19: Measurement of Toxoplasma gondii tachyzoite growth in supplemented media, or supplemented media with 0.1% (v/v) DMSO (solvent control for drugs) over one week. Parasites were in the mid-logarithmic phase of growth at 72 hours post seeding (arrow), hence this timepoint was used for the experiment outlined in FIG. 16E. Shown are mean values (±S.D.) n=4 independent experiments.

FIG. 20A shows the structure of metronidazole alone (i), metronidazole coupled to steroid (via a linker at C17) (BC62D) (ii), and metronidazole coupled to a linker (BC61D).

FIG. 20B shows dose response curves of metronidazole alone, metronidazole coupled to steroid (via a linker), and metronidazole coupled to a linker, showing the effect of these compounds on asexual P. falciparum stages.

FIG. 21A shows the structure of hydroxychloroquine alone (i), hydroxychloroquine coupled to steroid (via a linker at C17) (BC75D) (ii), hydroxychloroquine coupled to a linker (BC72D) (iii), and steroid & linker (HJB8a53) (iv).

FIG. 21B shows dose response curves of hydroxychloroquine alone and hydroxychloroquine coupled to steroid (via a linker) (DHEA-hydroxychloroquine conjugate (BC75D)), showing the effect of these compounds on asexual P. falciparum stages of the chloroquine sensitive strain 3D7.

FIG. 21C shows dose response curves of hydroxychloroquine alone and hydroxychloroquine coupled to steroid (via a linker) (DHEA-hydroxychloroquine conjugate (BC75D)), showing the effect of these compounds on asexual P. falciparum stages of the chloroquine sensitive strain C2_GC03 (which is genetically modified to convert the chloroquine-resistant P. falciparum strain Dd2 to become chloroquine sensitive).

FIG. 21D shows dose response curves (72 hours) of hydroxychloroquine alone and hydroxychloroquine coupled to steroid (via a linker) (DHEA-hydroxychloroquine conjugate (BC75D)), showing the effect of these compounds on asexual P. falciparum stages of the chloroquine resistant strain C4_Dd2.

FIG. 21E shows dose response curves (72 hours) against human embryonic kidney (HEK293) cell viability incubated with hydroxychloroquine, DHEA-hydroxychloroquine conjugate (BC75D), hydroxychloroquine-linker (BC72D), and DHEA linker (HJB8a53).

FIG. 22 shows dose response curves showing the effects of: (i) primaquine, (ii) steroid-primaquine conjugate BCSB over 72 hours, and (iii) steroid-bound peroxide BC86D over 72 hours, on Leishmania tarentolae.

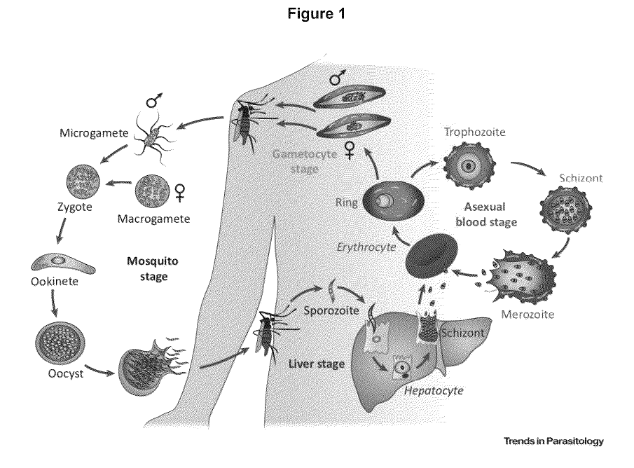

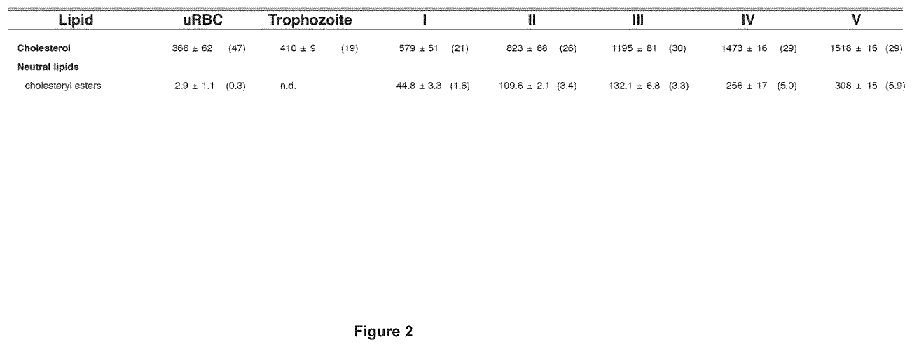

### DETAILED DESCRIPTION OF EMBODIMENTS OF THE INVENTION

Preferred embodiments of the present invention are described below by way of example only.

### Definitions

Unless otherwise herein defined, the following terms will be understood to have the general meanings which follow. The terms referred to below have the general meanings which follow when the term is used alone and when the term is used in combination with other terms, unless otherwise indicated. Hence, for example, the definition of “alkyl” applies to “alkyl” as well as the “alkyl” portions of “haloalkyl”, “heteroalkyl”, “arylalkyl” etc.

The term “alkyl” refers to a straight chain or branched chain saturated hydrocarbyl group. Unless indicated otherwise, preferred are C₁₋₆alkyl and C₁₋₄alkyl groups. The term “C_{x-y}alkyl”, where x and y are integers, refers to an alkyl group having x to y carbon atoms. For example, the term “C₁₋₆alkyl” refers to an alkyl group having 1 to 6 carbon atoms. Examples of C₁₋₆alkyl include methyl (Me), ethyl (Et), propyl (Pr), isopropyl (i-Pr), butyl (Bu), isobutyl (i-Bu), sec-butyl (s-Bu), tert-butyl (t-Bu), pentyl, neopentyl, hexyl and the like. Unless the context requires otherwise, the term “alkyl” also encompasses alkyl groups containing one less hydrogen atom such that the group is attached via two positions, i.e. divalent.

The term “alkenyl” refers to a straight chain or branched chain hydrocarbyl group having at least one double bond of either E- or Z-stereochemistry where applicable. Unless indicated otherwise, preferred are C₂₋₆alkenyl and C₂₋₃alkenyl groups. The term “C_{x-y}alkenyl”, where x and y are integers, refers to an alkenyl group having x to y carbon atoms. For example, the term “C₂₋₆alkenyl” refers to an alkenyl group having 2 to 6 carbon atoms. Examples of C₂₋₆alkenyl include vinyl, 1-propenyl, 1- and 2-butenyl and 2-methyl-2-propenyl. Unless the context requires otherwise, the term “alkenyl” also encompasses alkenyl groups containing one less hydrogen atom such that the group is attached via two positions, i.e. divalent.

The term “alkynyl” refers to a straight chain or branched chain hydrocarbyl group having at least one triple bond. Unless indicated otherwise, preferred are C₂₋₆alkynyl and C₂₋₃alkynyl groups. The term “C_{x-y}alkynyl”, where x and y are integers, refers to an alkynyl group having x to y carbon atoms. For example, the term “C₂₋₆alkynyl” refers to an alkynyl group having 2 to 6 carbon atoms. Examples of C₂₋₆alkynyl include ethynyl, 1-propynyl, 1- and 2-butynyl, 2-pentynyl, 3-pentynyl, 4-pentynyl, 2-hexynyl, 3-hexynyl, 4-hexynyl and 5-hexynyl and the like. Unless the context indicates otherwise, the term “alkynyl” also encompasses alkynyl groups containing one less hydrogen atom such that the group is attached via two positions, i.e. divalent.

The term “C₃₋₈cycloalkyl” refers to a non-aromatic cyclic hydrocarbyl group having from 3 to 8 carbon atoms. Such groups include cyclopropyl, cyclobutyl, cyclopentyl, cyclohexyl, cycloheptyl and cyclooctyl. The term “C₃₋₈cycloalkyl” encompasses groups where the cyclic hydrocarbyl group is saturated such as cyclohexyl or unsaturated such as cyclohexenyl. C₃₋₆cycloalkyl such as cyclopropyl, cyclobutyl, cyclopentyl and cyclohexyl are preferred.

The term “hydroxy” refers to the group —OH.

The term “oxo” refers to the group ═O.

The term “heteroalkyl” refers to an alkyl group as defined above covalently bound via a heteroatom linkage (e.g. via O, N or S). Examples where the “heteroalkyl” group is covalently bound to an SP³ carbon include ethers (e.g. alkoxy), thioethers and alkylamino groups. Unless indicated otherwise, preferred are C₁₋₆heteroalkyl, C₁₋₄heteroalkyl and C₁₋₃heteroalkyl groups.

The term “alkoxy” refers to an alkyl group as defined above covalently bound via an O linkage, such as methoxy, ethoxy, propoxy, isopropoxy, butoxy, tert-butoxy and pentoxy. Unless indicated otherwise, preferred are C₁₋₆alkoxy, C₁₋₄alkoxy and C₁₋₃alkoxy groups.

The term “haloalkyl” refers to a C₁₋₆alkyl which is substituted with one or more halogens, such as, for example, —CHF₂, —CH₂CF₃, —CF₂CF₃ and —CF₃. Unless indicated otherwise, haloC₁₋₃ alkyl groups are preferred.

The term “haloC₁₋₆alkoxy” refers to a C₁₋₆alkoxy which is substituted with one or more halogens. HaloC₁₋₃alkoxy groups are preferred, such as, for example, —OCHF₂ and —OCF₃.

The term “carboxylate” or “carboxyl” refers to the group —COO⁻ or —COOH.

The term “ester” refers to a carboxyl group having the hydrogen replaced with, for example, an alkyl group (“alkylester” or “alkyloxycarbonyl”), an aryl or aralkyl group (“arylester” or “aralkylester”) and so on. CO₂C₁₋₃alkyl groups are preferred, such as for example, methylester (—CO₂Me), ethylester (—CO₂Et) and propylester (—CO₂Pr) and reverse esters thereof (e.g. —OC(O)Me, —OC(O)Et and —OC(O)Pr).

The term “cyano” refers to the group —CN.

The term “nitro” refers to the group —NO₂.

The term “amino” refers to the group —NH₂.

The term “substituted amino” or “secondary amino” refers to an amino group having a hydrogen replaced with, for example, an alkyl group (“alkylamino”), an aryl or aralkyl group (“arylamino”, “aralkylamino”) and so on. C₁₋₃alkylamino groups are preferred, such as for example, methylamino (—NHMe), ethylamino (—NHEt) and propylamino (—NHPr).

The term “disubstituted amino” or “tertiary amino” refers to an amino group having the two hydrogens replaced with, for example, an alkyl group, which may be the same or different (“di(alkyl)amino”), an aryl and alkyl group (“aryl(alkyl)amino”) and so on. Di(C₁₋₃alkyl)amino groups are preferred, such as, for example, dimethylamino (—NMe₂), diethylamino (—NEt₂), dipropylamino (—NPr₂) and variations thereof (e.g. —N(Me)(Et) and so on).

The term “acyl” or “aldehyde” refers to the group —C(═O)H.

The term “substituted acyl” or “ketone” refers to an acyl group having the hydrogen replaced with, for example, an alkyl group (“alkylacyl” or “alkylketone”), an aryl group (“arylketone”), an aralkyl group (“aralkylketone”) and so on. Unless indicated otherwise, C₁₋₆ alkylacyl and C₁₋₃alkylacyl groups are preferred.

The term “amido” or “amide” refers to the group —C(O)NH₂.

The term “aminoacyl” refers to the group —NHC(O)H.

The term “substituted amido” or “substituted amide” refers to an amido group having a hydrogen replaced with, for example, an alkyl group (“alkylamido” or “alkylamide”), an aryl (“arylamido”), aralkyl group (“aralkylamido”) and so on. C₁₋₃alkylamide groups are preferred, such as, for example, methylamide (—C(O)NHMe), ethylamide (—C(O)NHEt) and propylamide (—C(O)NHPr) and reverse amides thereof (e.g. —NHC(O)Me, —NHC(O)Et and —NHC(O)Pr).

The term “disubstituted amido” or “disubstituted amide” refers to an amido group having the two hydrogens replaced with, for example, an alkyl group (“di(alkyl)amido” or “di(alkyl)amide”), an aralkyl and alkyl group (“alkyl(aralkyl)amido”) and so on. Di(C₁₋₃alkyl)amide groups are preferred, such as, for example, dimethylamide (—C(O)NMe₂), diethylamide (—C(O)NEt₂) and dipropylamide (—C(O)NPr₂) and variations thereof (e.g. —C(O)N(Me)Et and so on) and reverse amides thereof.

The term “thiol” refers to the group —SH.

The term “C₁₋₆alkytthio” refers to a thiol group having the hydrogen replaced with a C₁₋₆alkyl group. C₁₋₃alkylthio groups are preferred, such as, for example, thiolmethyl, thiolethyl and thiolpropyl.

The term “thioxo” refers to the group ═S.

The term “sulfiny” refers to the group —S(═O)H.

The term “substituted sulfinyl” or “sulfoxide” refers to a sulfinyl group having the hydrogen replaced with, for example, a C₁₋₆alkyl group (“C₁₋₆alkylsulfinyl” or “C₁₋₆alkylsulfoxide”), an aryl (“arylsulfinyl”), an aralkyl (“aralkylsulfinyl”) and so on. C₁₋₃alkylsulfinyl groups are preferred, such as, for example, —SOmethyl, —SOethyl and —SOpropyl.

The term “sulfonyl” refers to the group —SO₂H.

The term “substituted sulfonyl” refers to a sulfonyl group having the hydrogen replaced with, for example, a C₁₋₆alkyl group (“C₁₋₆alkylsulfonyl”), an aryl (“arylsulfonyl”), an aralkyl (“aralkylsulfonyl”) and so on. SulfonylC₁₋₃alkyl groups are preferred, such as, for example, —SO₂Me, —SO₂Et and —SO₂Pr.

The term “sulfonylamido” or “sulfonamide” refers to the group —SO₂NH₂.

The term “substituted sulfonamido” or “substituted sulphonamide” refers to a sulfonylamido group having a hydrogen replaced with, for example, a C₁₋₆alkyl group (“C₁₋₆alkylsulfonylamido”), an aryl (“arylsulfonamide”), aralkyl (“aralkylsulfonamide”) and so on. C₁₋₃alkylsulfonylamido groups are preferred, such as, for example, —SO₂NHMe, —SO₂NHEt and —SO₂NHPr and reverse sulfonamides thereof (e.g. —NHSO₂Me, —NHSO₂Et and —NHSO₂Pr).

The term “disubstituted sulfonamido” or “disubstituted sulphonamide” refers to a sulfonylamido group having the two hydrogens replaced with, for example, a C₁₋₆alkyl group, which may be the same or different (“di(C₁₋₆alkyl)sulfonylamido”), an aralkyl and alkyl group (“aralkyl(alkyl)sulfonamido”) and so on. Di(C₁₋₃alkyl)sulfonylamido groups are preferred, such as, for example, —SO₂NMe₂, —SO₂NEt₂ and —SO₂NPr₂ and variations thereof (e.g. —SO₂N(Me)Et and so on) and reverse sulfonamides thereof.

The term “sulfate” refers to the group —OS(O)₂OH and includes groups having the hydrogen replaced with, for example, a C₁₋₈alkyl group (“C₁₋₆alkylsulfate”), an aryl (“arylsulfate”), an aralkyl (“aralkylsulfate”) and so on. C₁₋₃alkylsulfates are preferred, such as, for example, —OS(O)₂OMe, —OS(O)₂OEt and —OS(O)₂OPr.

The term “sulfonate” refers to the group —SO₃H and includes groups having the hydrogen replaced with, for example, a C₁₋₆alkyl group (“C₁₋₆alkylsulfonate”), an aryl (“arylsulfonate”), an aralkyl (“aralkylsulfonate”) and so on. C₁₋₃alkylsulfonates are preferred, such as, for example, —SO₃Me, —SO₃Et and —SO₃Pr.

The term “aryl” refers to a carbocyclic (non-heterocyclic) aromatic ring or mono-, bi- or tri-cyclic ring system. The aromatic ring or ring system is generally composed of 6 to 10 carbon atoms. Examples of aryl groups include but are not limited to phenyl, biphenyl, naphthyl and tetrahydronaphthyl. 6-membered aryls such as phenyl are preferred. The term “arylalkyl” or “aralkyl” refers to an arylC₁₋₆alkyl- such as benzyl.

The term “arylalkoxy” refers to arylC₁₋₆alkoxy- such as benzyloxy.

The term “heterocyclyl” refers to a moiety obtained by removing a hydrogen atom from a ring atom of a heterocyclic compound which moiety has from 3 to 10 ring atoms (unless otherwise specified), of which 1, 2, 3 or 4 are ring heteroatoms, each heteroatom being independently selected from O, S and N, and the remainder of the ring atoms are carbon atoms. The term “heterocycloalkyl” refers to a heterocyclyl moiety comprising a saturated cyclic group comprising one or more ring carbons and one or more ring heteroatoms. “Heterocycloalkenyl” refers to a heterocyclyl moiety comprising a cyclic group comprising at least one carbon-carbon double bond and one or more ring heteroatoms. “Heterocycloalkynyl” refers to a heterocyclyl moiety comprising a cyclic group comprising at least one carbon-carbon triple bond and one or more ring heteroatoms.

In this context, the prefixes 3-, 4-, 5-, 6-, 7-, 8-, 9- and 10-membered denote the number of ring atoms, or range of ring atoms, whether carbon atoms or heteroatoms. For example, the term “3-10-membered heterocylyl”, as used herein, refers to a heterocyclyl group having 3, 4, 5, 6, 7, 8, 9 or 10 ring atoms. Examples of heterocylyl groups include 5-6-membered monocyclic heterocyclyls and 9-10 membered fused bicyclic heterocyclyls.

Examples of monocyclic heterocyclyl groups include, but are not limited to, those containing one nitrogen atom such as aziridine (3-membered ring), azetidine (4-membered ring), pyrrolidine (tetrahydropyrrole), pyrroline (e.g., 3-pyrroline, 2,5-dihydropyrrole), 2H-pyrrole or 3H-pyrrole (isopyrrole, isoazole) or pyrrolidinone (5-membered rings), piperidine, dihydropyridine, tetrahydropyridine (6-membered rings), and azepine (7-membered ring); those containing two nitrogen atoms such as imidazoline, pyrazolidine (diazolidine), pyrazoline (dihydropyrazole) (5-membered rings), piperazine (6-membered ring); those containing one oxygen atom such as oxirane (3-membered ring), oxetane (4-membered ring), oxolane (tetrahydrofuran), oxole (dihydrofuran) (5-membered rings), oxane (tetrahydropyran), dihydropyran, pyran (6-membered rings), oxepin (7-membered ring); those containing two oxygen atoms such as dioxolane (5-membered ring), dioxane (6-membered ring), and dioxepane (7-membered ring); those containing three oxygen atoms such as trioxane (6-membered ring); those containing one sulfur atom such as thiirane (3-membered ring), thietane (4-membered ring), thiolane (tetrahydrothiophene) (5-membered ring), thiane (tetrahydrothiopyran) (6-membered ring), thiepane (7-membered ring); those containing one nitrogen and one oxygen atom such as tetrahydrooxazole, dihydrooxazole, tetrahydroisoxazole, dihydroisoxazole (5-membered rings), morpholine, tetrahydrooxazine, dihydrooxazine, oxazine (6-membered rings); those containing one nitrogen and one sulfur atom such as thiazoline, thiazolidine (5-membered rings), thiomorpholine (6-membered ring); those containing two nitrogen and one oxygen atom such as oxadiazine (6-membered ring); those containing one oxygen and one sulfur such as: oxathiole (5-membered ring) and oxathiane (thioxane) (6-membered ring); and those containing one nitrogen, one oxygen and one sulfur atom such as oxathiazine (6-membered ring).

The term “heterocyclyl” encompasses aromatic heterocyclyls and non-aromatic heterocyclyls.

The term “aromatic heterocyclyl” may be used interchangeably with the term “heteroaromatic” or the term “heteroaryl” or “hetaryl”. The heteroatoms in the aromatic heterocyclyl group may be independently selected from N, S and O.

“Heteroaryl” is used herein to denote a heterocyclic group having aromatic character and embraces aromatic monocyclic ring systems and polycyclic (e.g. bicyclic) ring systems containing one or more aromatic rings. The term aromatic heterocyclyl also encompasses pseudoaromatic heterocyclyls. The term “pseudoaromatic” refers to a ring system which is not strictly aromatic, but which is stabilised by means of delocalisation of electrons and behaves in a similar manner to aromatic rings. The term aromatic heterocyclyl therefore covers polycyclic ring systems in which all of the fused rings are aromatic as well as ring systems where one or more rings are non-aromatic, provided that at least one ring is aromatic. In polycyclic systems containing both aromatic and non-aromatic rings fused together, the group may be attached to another moiety by the aromatic ring or by a non-aromatic ring.

Examples of heteroaryl groups are monocyclic and bicyclic groups containing from five to ten ring members. The heteroaryl group can be, for example, a five membered or six membered monocyclic ring or a bicyclic structure formed from fused five and six membered rings or two fused six membered rings or two fused five membered rings. Each ring may contain up to four heteroatoms selected from nitrogen, sulfur and oxygen. The heteroaryl group can contain up to 4 heteroatoms, more typically up to 3 heteroatoms, more usually up to 2 heteroatoms. In one embodiment, the heteroaryl group contains at least one ring nitrogen atom. The nitrogen atoms in the heteroaryl group can be basic, as in the case of an imidazole or pyridine, or essentially non-basic as in the case of an indole or pyrrole nitrogen. In general, the number of basic nitrogen atoms present in the heteroaryl group, including any amino group substituents of the ring, will be less than five.

Aromatic heterocyclyl groups may be 5-membered or 6-membered mono-cyclic aromatic ring systems.

Examples of 5-membered monocyclic heteroaryl groups include but are not limited to furanyl, thienyl, pyrrolyl, oxazolyl, oxadiazolyl (including 1,2,3- and 1,2,4-oxadiazolyls and furazanyl, i.e. 1,2,5-oxadiazolyl), thiazolyl, isoxazoyl, isothiazolyl, pyrazoyl, imidazoyl, triazolyl (including 1,2,3-, 1,2,4- and 1,3,4-triazolyls), oxatriazolyl, tetrazolyl, thiadiazolyl (including 1,2,3- and 1,3,4-thiadiazolyls) and the like.

Examples of 6-membered monocyclic heteroaryl groups include but are not limited to pyridinyl, pyrimidinyl, pyridazinyl, pyrazinyl, triazinyl, pyranyl, oxazinyl, dioxinyl, thiazinyl, thiadiazinyl and the like. Examples of 6-membered aromatic heterocyclyls containing nitrogen include pyridyl (1 nitrogen), pyrazinyl, pyrimidinyl and pyridazinyl (2 nitrogens).

Aromatic heterocyclyl groups may also be bicyclic or polycyclic heteroaromatic ring systems such as fused ring systems (including purine, pteridinyl, naphthyridinyl, 1H-thieno[2,3-c]pyrazolyl, thieno[2,3-b]furyl and the like) or linked ring systems (such as oligothiophene, polypyrrole and the like). Fused ring systems may also include aromatic 5-membered or 6-membered heterocyclyls fused to carbocyclic aromatic rings such as phenyl, naphthyl, indenyl, azulenyl, fluorenyl, anthracenyl and the like, such as 5-membered aromatic heterocyclyls containing nitrogen fused to phenyl rings, 5-membered aromatic heterocyclyls containing 1 or 2 nitrogens fused to phenyl ring.

A bicyclic heteroaryl group may be, for example, a group selected from: a) a benzene ring fused to a 5- or 6-membered ring containing 1, 2 or 3 ring heteroatoms; b) a pyridine ring fused to a 5- or 6-membered ring containing 1, 2 or 3 ring heteroatoms; c) a pyrimidine ring fused to a 5- or 6-membered ring containing 1 or 2 ring heteroatoms; d) a pyrrole ring fused to a 5- or 6-membered ring containing 1, 2 or 3 ring heteroatoms; e) a pyrazole ring fused to a 5- or 6-membered ring containing 1 or 2 ring heteroatoms; f) an imidazole ring fused to a 5- or 6-membered ring containing 1 or 2 ring heteroatoms; g) an oxazole ring fused to a 5- or 6-membered ring containing 1 or 2 ring heteroatoms; h) an isoxazole ring fused to a 5- or 6-membered ring containing 1 or 2 ring heteroatoms; i) a thiazole ring fused to a 5- or 6-membered ring containing 1 or 2 ring heteroatoms; j) an isothiazole ring fused to a 5- or 6-membered ring containing 1 or 2 ring heteroatoms; k) a thiophene ring fused to a 5- or 6-membered ring containing 1, 2 or 3 ring heteroatoms; l) a furan ring fused to a 5- or 6-membered ring containing 1, 2 or 3 ring heteroatoms; m) a cyclohexyl ring fused to a 5- or 6-membered aromatic ring containing 1, 2 or 3 ring heteroatoms; and n) a cyclopentyl ring fused to a 5- or 6-membered aromatic ring containing 1, 2 or 3 ring heteroatoms.

Particular examples of bicyclic heteroaryl groups containing a five membered ring fused to another five membered ring include but are not limited to imidazothiazole (e.g. imidazo[2,1-b]thiazole) and imidazoimidazole (e.g. imidazo[1,2-a]imidazole).

Particular examples of bicyclic heteroaryl groups containing a six membered ring fused to a five membered ring include but are not limited to benzofuran, benzothiophene, benzimidazole, benzoxazole, isobenzoxazole, benzisoxazole, benzothiazole, benzisothiazole, isobenzofuran, indole, isoindole, indolizine, indoline, isoindoline, purine (e.g., adenine, guanine), indazole, pyrazolopyrimidine (e.g. pyrazolo[1,5-a]pyrimidine), benzodioxole and pyrazolopyridine (e.g. pyrazolo[1,5-a]pyridine) groups. A further example of a six membered ring fused to a five membered ring is a pyrrolopyridine group such as a pyrrolo[2,3-b]pyridine group.

Particular examples of bicyclic heteroaryl groups containing two fused six membered rings include, but are not limited to, quinoline, isoquinoline, chroman, thiochroman, chromene, isochromene, isochroman, benzodioxan, quinolizine, benzoxazine, benzodiazine, pyridopyridine, quinoxaline, quinazoline, cinnoline, phthalazine, naphthyridine and pteridine groups.

Examples of heteroaryl groups containing an aromatic ring and a non-aromatic ring include tetrahydroisoquinoline, tetrahydroquinoline, dihydrobenzothiophene, dihydrobenzofuran, 2,3-dihydro-benzo[1,4]dioxine, benzo[1,3]dioxole, 4,5,6,7-tetrahydrobenzofuran, indoline and isoindoline groups.

Examples of aromatic heterocyclyls fused to carbocyclic aromatic rings may therefore include, but are not limited to, benzothiophenyl, indolyl, isoindolyl, benzofuranyl, isobenzofuranyl, benzimidazolyl, indazolyl, benzoxazolyl, benzisoxazolyl, isobenzoxazoyl, benzothiazolyl, benzisothiazolyl, quinolinyl, isoquinolinyl, quinoxalinyl, quinazolinyl, cinnolinyl, benzotriazinyl, phthalazinyl, carbolinyl and the like.

The term “non-aromatic heterocyclyl” encompasses saturated and unsaturated rings which contain at least one heteroatom selected from the group consisting of N, S and O.

Non-aromatic heterocyclyls may be 3-7 membered mono-cyclic rings.

Examples of 5-membered non-aromatic heterocyclyl rings include 2H-pyrrolyl, 1-pyrrolinyl, 2-pyrrolinyl, 3-pyrrolinyl, pyrrolidinyl, 1-pyrrolidinyl, 2-pyrrolidinyl, 3-pyrrolidinyl, tetrahydrofuranyl, tetrahydrothiophenyl, pyrazolinyl, 2-pyrazolinyl, 3-pyrazolinyl, pyrazolidinyl, 2-pyrazolidinyl, 3-pyrazolidinyl, imidazolidinyl, 3-dioxalanyl, thiazolidinyl, isoxazolidinyl, 2-imidazolinyl and the like.

Examples of 6-membered non-aromatic heterocyclyls include piperidinyl, piperidinonyl, pyranyl, dihyrdopyranyl, tetrahydropyranyl, 2H-pyranyl, 4H-pyranyl, thianyl, thianyl oxide, thianyl dioxide, piperazinyl, diozanyl, 1,4-dioxinyl, 1,4-dithianyl, 1,3,5-triozalanyl, 1,3,5-trithianyl, 1,4-morpholinyl, thiomorpholinyl, 1,4-oxathianyl, triazinyl, 1,4-thiazinyl and the like.

Examples of 7-membered non-aromatic heterocyclyls include azepanyl, oxepanyl, thiepanyl and the like.

Non-aromatic heterocyclyl rings may also be bicyclic heterocyclyl rings such as linked ring systems (for example uridinyl and the like) or fused ring systems. Fused ring systems include non-aromatic 5-membered, 6-membered or 7-membered heterocyclyls fused to non-aromatic carbocyclic rings.

The term “halo” refers to fluoro, chloro, bromo or iodo.

Unless otherwise defined, the term “optionally substituted”, or “substituted or unsubstituted”, as used herein indicates a group may or may not be substituted with 1, 2, 3, 4 or more groups, preferably 1, 2 or 3 groups, more preferably 1 or 2 groups, independently selected from the group consisting of alkyl (e.g. C₁₋₆alkyl), alkenyl (e.g. C₂₋₆alkenyl), alkynyl (e.g. C₂₋₆ alkynyl), cycloalkyl (e.g. C₁₋₆cycloalkyl), hydroxyl, oxo, heteroalkyl, alkoxy (e.g. C₁₋₆alkoxy), aryloxy, arylC₁₋₆alkoxy, halo, haloC₁₋₆alkyl (such as —CF₃ and —CHF₂), haloC₁₋₆alkoxy (such as —OCF₃ and —OCHF₂), carboxyl, esters, cyano, nitro, amino, substituted amino, disubstituted amino, acyl, substituted acyl, ketones, amides, aminoacyl, substituted amides, disubstituted amides, thiol, alkylthio, thioxo, sulfates, sulfonates, sulfinyl, substituted sulfinyl, sulfonyl, substituted sulfonyl, sulfonylamides, substituted sulfonamides, disubstituted sulfonamides, aryl, arylC₁₋₆alkyl, heterocyclylC₁₋₆alkyl, arylC₂₋₆alkenyl, heterocyclylC₂₋₆alkenyl, arylC₂₋₆alkynyl, heterocyclylC₂₋₆alkynyl, heteroarylC₁₋₆alkyl, heteroarylC₂₋₆alkenyl, heteroarylC₂₋₆alkynyl, heterocyclyl and heteroaryl, wherein each alkyl, alkenyl, alkynyl, cycloalkyl, aryl and heterocyclyl and groups containing them may be further optionally substituted. Optional substituents in the case of heterocycles containing N may also include but are not limited to C₁₋₆ alkyl i.e. N—C₁₋₆alkyl.

For optionally substituted “alkyl”, “alkenyl” and “alkynyl”, the optional substituent or substituents are preferably selected from amino, substituted amino, disubstituted amino, aryl, halo (e.g. F, Cl, Br, I), heterocyclyl, C₃₋₈cycloalkyl, C₁₋₆alkoxy, hydroxyl, oxo, aryloxy, haloC₁₋₆ alkyl, haloC₁₋₆alkoxyl and carboxyl. Each of these optional substituents may also be optionally substituted with any of the optional substituents referred to above, where nitro, amino, substituted amino, cyano, heterocyclyl (including non-aromatic heterocyclyl and heteroaryl), C₁₋₆alkyl, C₂₋₆alkenyl, C₂₋₆alkynyl, C₁₋₆alkoxyl, haloC₁₋₆alkyl, haloC₁₋₆alkoxy, halo, hydroxyl and carboxyl are preferred.

For substituted or unsubstituted “ammonium”, the optional substituent or substituents are preferably selected from C₁₋₃alkyl, such as C₁alkyl, C₂alkyl or C₃alkyl. More preferably, the optional substituent or substituents are CH₃.

For substituted or unsubstituted “amino”, the optional substituent or substituents are preferably selected from C₁₋₃alkyl, such as C₁alkyl, C₂alkyl or C₃alkyl. More preferably, the optional substituent or substituents are CH₃.

It will be understood that suitable derivatives of aromatic heterocyclyls containing nitrogen include N-oxides thereof.

As used herein, a “subject” is any animal which can be susceptible to a parasitic infection. The animal can be, for example, a human, a non-human primate, poultry, sheep, dog, cat, cattle, horse, cow, pig, goat, or any other animals which can suffer from a parasitic infection. In one embodiment, the animal is a human. In another embodiment, the animal is a non-human animal.

As used herein, “treating” means affecting a subject, tissue or cell to obtain a desired pharmacological and/or physiological effect and includes inhibiting the condition, i.e. arresting its development; or relieving or ameliorating the effects of the condition i.e. cause reversal or regression of the effects of the condition.

As used herein, “preventing” means preventing a condition from occurring in a cell or subject that may be at risk of having the condition, but does not necessarily mean that condition will not eventually develop, or that a subject will not eventually develop a condition. Preventing includes delaying the onset of a condition in a cell or subject.

Except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

### Drug Delivery System

The present invention broadly relates to a drug delivery system formed by conjugating a steroid to a biologically active compound via an optional linker, referred to herein as a “steroid-active conjugate”.

In one form, the steroid-active conjugate comprises a steroid conjugated to an anti-parasitic moiety via an optional linker, at C3, C16 or C17, typically C16 or C17, more typically C17, of the steroid.

The present invention also relates broadly to a method of producing a steroid-active conjugate comprising coupling a steroid to a biologically active compound, such as an anti-parasitic moiety, via an optional linker, at C3, C16 or C17, typically C16 or C17, more typically C17, of the steroid.

The inventors have found that fluorescent dyes linked, optionally via a linker group, to C17 of cholesterol are incorporated into apicomplexan parasites during infection of red blood cells much more effectively than the same fluorescent dye linked to C3 of cholesterol. The inventors therefore reasoned that drugs targeting parasites, such as anti-apicomplexan parasite drugs, could be effectively introduced into the parasites during infection by linking the anti-parasite drugs to C3 and/or C17 or C16 of a steroid, more typically C17 of a steroid.

As described herein, the inventors have linked anti-apicomplexan parasite drugs to steroids, such as the C17 of cholesterol, and have found that such drug-steroid conjugates are more effective at treating apicomplexan parasite infections than the unconjugated apicomplexan parasite drug. This means that less drug can be used to treat the apicomplexan parasite infection, and this may lead to cost savings. Use of less drug means that side effects of the apicomplexan parasite drug can be reduced or avoided. Use of the drug-steroid conjugates may provide efficacy against a parasite where the unconjugated drug is not effective against that parasite. Further, there may be improved safety characteristics from reduced toxicity towards host cells, and potential to increase the half-life of a drug by conjugating the drug to a steroid. Also, targeted delivery and use of less drug may result in a reduction in the emergence of drug-resistant organisms. The inventors hypothesis that existing drugs may be repurposed by linking the drug to a steroid compound to form a conjugated drug that is active against drug-resistant strains; desirably, the same concentration of conjugated drug could potentially kill the drug-resistant strain. Further, the present invention could enable a drug in the form of a conjugated drug to target multiple life-cycle stages at once, e.g. drugs against erythrocytic stages when combined are also targeting gametocytes or liver-stages at the same concentration due to an increase in efficacy.

Without wishing to be bound by theory, the inventors believe that coupling an anti-parasitic drug to a steroid at C3, C16 or C17 of the steroid may improve the efficacy and selectivity of a broad range of drugs with different anti-parasitic properties.

Steroid-active conjugates can therefore be used to treat or prevent parasite infections, such as apicomplexan parasite infections.

The steroid-active conjugate comprises a group G covalently bound via an optional linker group L to a steroid group J, represented schematically below:

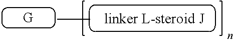

G is a group which has biological activity, such as anti-parasite activity (e.g. G may have anti-protozoan activity, such as anti-apicomplexan activity, or G may have anti-helminth activity). G may be a functional group, or G may be a substituent formed from a compound with anti-parasite activity (e.g. a drug radical formed from a drug with anti-parasite activity). For example, G may be a group containing a peroxide or hydroperoxide group, or G may be a radical formed from primaquine, artesunate, resorufin, hydroxychloroquine, sulfadiazine, metronidazole, amodiaquine, mefloquine, pyrimethamine, atovaquone, fosmidomycin, metrifonate, afromosin, medicarpin, flubendazole, mebendazole, or oxamniquine.

J is a steroid group which is bound to optional linker L at C17, C16 or C3 of steroid J. Examples of suitable steroids on which J may be based include DHEA, epiandrosterone, androsterone, etiocholanolone, estrone, cholesterol and testosterone, and derivatives thereof.

L is a linker group and may be bound at C17, C16 or C3 of the steroid J. In one embodiment, the linker L is bound to steroid J at C17 of the steroid J. In one embodiment, the linker L is bound to steroid J at C16 of the steroid J. In one embodiment, the linker L is bound to steroid J at C3 of the steroid J. Preferably, the linker L is bound to steroid J at C17 of the steroid J.

n may be 1, 2, 3, 4 or 5. Typically, n is 1 or 2. Preferably, n is 1.

In one embodiment, the steroid-active conjugate is a compound of Formula (I):

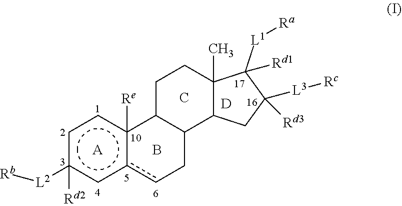

wherein:
each of R^{a}, R^{b} and R^{c} is independently selected from —H, —OH, ═O, substituted or unsubstituted —C₁₋₁₀ alkyl, substituted or unsubstituted —C₂₋₁₀ alkenyl, substituted or unsubstituted —C₂₋₁₀ alkynyl, substituted or unsubstituted —C₃₋₁₀ cycloalkyl, substituted or unsubstituted —C₅₋₁₄ cycloalkenyl, substituted or unsubstituted —C₈₋₁₄ cycloalkynyl, and an anti-parasite moiety; wherein at least one of R^{a}, R^{b} and R^{c} is an anti-parasite moiety;
provided that: when R^{a} is —OH, L¹ is absent; when R^{a} is ═O, L¹ and R^{d1} are absent; when R^{b} is —OH, L² is absent; when R^{b} is ═O, L² is absent, R^{d2} is absent, and the C2-C3 bond and the C3-C4 bond are single bonds; when R^{c} is —OH, L³ is absent; when R^{c} is ═O, L³ and R^{d3} are absent;
when R^{a}, R^{b} or R^{c} is substituted, R^{a}, R^{b} or R^{c} is substituted with one or more groups selected from Substituent Group A;
R^{d1}, when present, is H or is a group that, together with R^{a}, forms a substituted or unsubstituted ozonide ring or a substituted or unsubstituted tetraoxane ring, wherein the optional substituent may be a —C₃₋₁₀ cycloalkyl; provided that when R^{d1} and R^{a} form a substituted or unsubstituted ozonide ring or a substituted or unsubstituted tetraoxane ring, L¹ is absent; wherein, when the ozonide or tetraoxane ring is substituted, the ozonide or tetraoxane ring is substituted with one or more —C₃₋₁₀ cycloalkyl groups;
R^{d2}, when present, is H; provided that when R^{d2} is present, the C2-C3 bond and the C3-C4 bond are single bonds;
R^{d3}, when present, is H or is a group that, together with R^{c}, forms a substituted or unsubstituted ozonide ring or a substituted or unsubstituted tetraoxane ring, wherein the optional substituent may be a —C₃₋₁₀ cycloalkyl; provided that when R^{d3} and R^{c} form a substituted or unsubstituted ozonide ring or a substituted or unsubstituted tetraoxane ring, L³ is absent; wherein, when the ozonide or tetraoxane ring is substituted, the ozonide or tetraoxane ring is substituted with one or more —C₃₋₁₀ cycloalkyl groups;
R^{e} is H or CH₃, or R^{e} is absent;
when present, L¹ is a group that provides a covalent linkage between R^{e} and the C17 of ring D;
when present, L² is a group that provides a covalent linkage between R^{b} and the C3 of ring A;
when present, L³ is a group that provides a covalent linkage between R^{c} and the C16 of ring D;
ring A may be a saturated or unsaturated ring, or ring A may be an aromatic ring, provided that when ring A is an aromatic ring, the C5-C6 bond is a single bond, R^{d2} is absent, R^{e} is absent, and R^{b} is not ═O;
[symbol] is a single bond or a double bond; provided that when the C4-C5 or C5-C10 bond is a double bond, the C5-C6 bond is a single bond; when the C5-C6 bond is a double bond, the C4-C5 and C5-C10 bonds are single bonds; and when the C3 forms a double bond with C2 or C4, R^{b} is not ═O;
or a stereoisomer thereof, or a salt thereof.

In some embodiments, Formula (I) may be selected from:

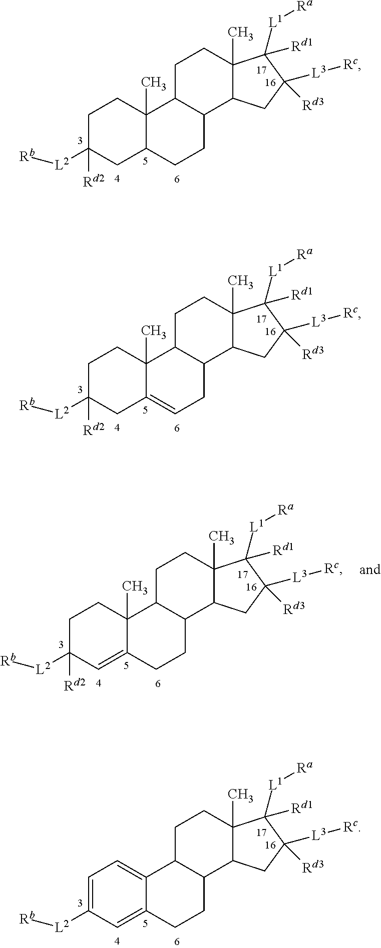

When R^{a}, R^{b} and R^{c} are not ═O, each of the groups -L¹-R^{a}, -L²-R^{b} and -L³-R^{c} may be attached in the α or β configuration on the respective ring. That is, the group may be positioned 3α- or 3β-of the steroid group, or the group may be positioned 16α- or 16β-of the steroid group, or the group may be positioned 17α- or 17β- or the steroid group.

In some embodiments, Formula (I) may be selected from:

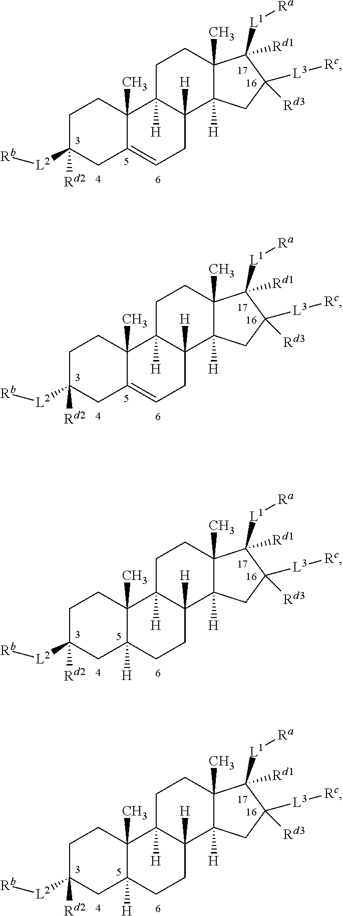

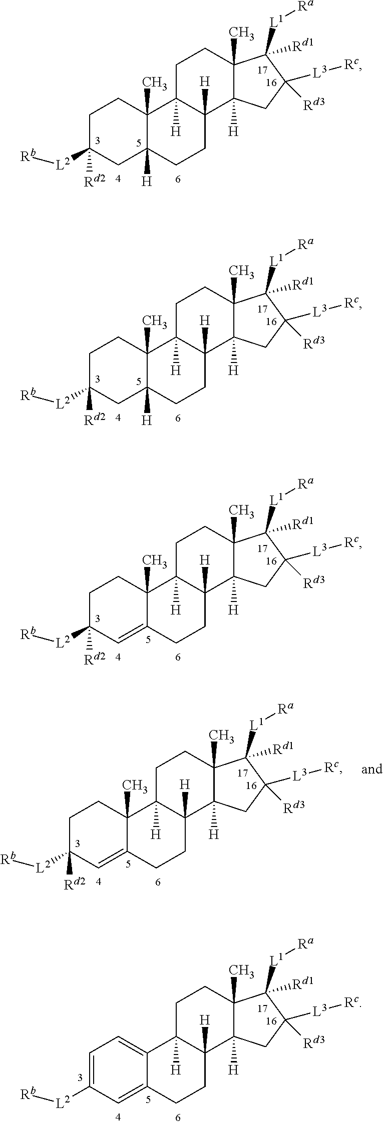

When R^{c} is not ═O in the above structures, -L³-R^{a} may be attached in the α or β configuration on the respective ring. That is, the group may be positioned 16α- or 16β-of the steroid group.

In some embodiments, each of R^{a}, R^{b} and R^{c} is independently selected from —H, —OH, ═O, substituted or unsubstituted —C₁₋₁₀ alkyl, substituted or unsubstituted —C₂₋₁₀ alkenyl, substituted or unsubstituted —C₂₋₁₀ alkynyl, substituted or unsubstituted —C₃₋₁₀ cycloalkyl, substituted or unsubstituted —C₅₋₁₄ cycloalkenyl, or substituted or unsubstituted —C₈₋₁₄ cycloalkynyl. In some embodiments, R^{a}, R^{b} or R^{c} is —OH. In some embodiments, R^{a}, R^{b} or R^{c} is ═O. In some embodiments, R^{a}, R^{b} or R^{c} is substituted or unsubstituted —C₁₋₁₀ alkyl. In some embodiments, R^{a}, R^{b} or R^{c} is substituted or unsubstituted —C₂₋₁₀ alkenyl. In some embodiments, R^{a}, R^{b} or R^{c} is substituted or unsubstituted —C₂₋₁₀ alkynyl. In some embodiments, R^{a}, R^{b} or R^{c} is substituted or unsubstituted —C₃₋₁₀ cycloalkyl. In some embodiments, R^{a}, R^{b} or R^{c} is substituted or unsubstituted —C₅₋₁₄ cycloalkenyl. In some embodiments, R^{a}, R^{b} or R^{c} is substituted or unsubstituted —C₈₋₁₄ cycloalkynyl. When R^{a}, R^{b} or R^{c} is substituted, R^{a}, R^{b} or R^{c} is substituted with one or more groups selected from Substituent Group A.

In some embodiments, R^{a}, R^{b} or R^{c} is substituted or unsubstituted —C₁₋₁₀ alkyl, being a straight chain or branched chain saturated hydrocarbyl group having from 1 to 10 carbon atoms. In some embodiments, R^{a}, R^{b} or R^{c} is —C₁₋₄ alkyl. In some embodiments, R^{a}, R^{b} or R^{c} is —C₁₋₆ alkyl. In some embodiments, R^{a}, R^{b} or R^{c} is —C₁₋₆ alkyl. In some embodiments, R^{a}, R^{b} or R^{c} is —C₁₋₇ alkyl. In some embodiments, R^{a}, R^{b} or R^{c} is —C₁₋₈ alkyl. Examples of —C₁₋₆ alkyl include methyl (Me), ethyl (Et), propyl (Pr), isopropyl (i-Pr), butyl (Bu), isobutyl (i-Bu), sec-butyl (s-Bu), tert-butyl (t-Bu), pentyl, neopentyl, hexyl, and the like. In some embodiments, R^{a}, R^{b} or R^{c} is substituted or unsubstituted —CH(CH₃)(CH₂)₃CH(CH₃)₂. In some embodiments, R^{a}, R^{b} or R^{c} is —CH(CH₃)(CH₂)₃CH(CH₃)₂. When R^{a}, R^{b} or R^{c} is substituted, R^{a}, R^{b} or R^{c} is substituted with one or more groups selected from Substituent Group A.

In some embodiments, R^{a}, R^{b} or R^{c} is substituted or unsubstituted —C₂₋₁₀ alkenyl, being a straight chain or branched chain unsaturated hydrocarbyl group having from 2 to 10 carbon atoms, and one, two, three or more double bonds. In some embodiments, R^{a}, R^{b} or R^{c} is —C₂₋₄alkenyl. In some embodiments, R^{a}, R^{b} or R^{c} is —C₂₋₅ alkenyl. In some embodiments, R^{a}, R^{b} or R^{c} is —C₂₋₆ alkenyl. In some embodiments, R^{a}, R^{b} or R^{c} is —C₂₋₇ alkenyl. In some embodiments, R^{a}, R^{b} or R^{c} is —C₂₋₈ alkenyl. Examples of —C₂₋₆ alkenyl include ethenyl, propenyl, butenyl, pentenyl, hexenyl, and the like. When R^{a}, R^{b} or R^{c} is substituted, R^{a}, R^{b} or R^{c} is substituted with one or more groups selected from Substituent Group A.

In some embodiments, R^{a}, R^{b} or R^{c} is substituted or unsubstituted —C₂₋₁₀ alkynyl, being a straight chain or branched chain unsaturated hydrocarbyl group having from 2 to 10 carbon atoms, and one, two, three or more triple bonds. In some embodiments, R^{a}, R^{b} or R^{c} is —C₂₋₄ alkynyl. In some embodiments, R^{a}, R^{b} or R^{c} is —C₂₋₅ alkynyl. In some embodiments, R^{a}, R^{b} or R^{c} is —C₂₋₆ alkynyl. In some embodiments, R^{a}, R^{b} or R^{c} is —C₂₋₇ alkynyl. In some embodiments, R^{a}, R^{b} or R^{c} is —C₂₋₈ alkynyl. Examples of —C₂₋₆ alkynyl include ethynyl, propynyl, butynyl, pentynyl, hexynyl, and the like. When R^{a}, R^{b} or R^{c} is substituted, R^{a}, R^{b} or R^{c} is substituted with one or more groups selected from Substituent Group A.

In some embodiments, R^{a}, R^{b} or R^{c} is substituted or unsubstituted —C₃₋₁₀ cycloalkyl being a non-aromatic cyclic hydrocarbyl group having from 3 to 10 carbon atoms. In some embodiments, R^{a}, R^{b} or R^{c} is —C₃₋₆ cycloalkyl. In some embodiments, R^{a}, R^{b} or R^{c} is cyclopropyl, cyclobutyl, cyclopentyl or cyclohexyl. When R^{a}, R^{b} or R^{c} is substituted, R^{a}, R^{b} or R^{c} is substituted with one or more groups selected from Substituent Group A.

In some embodiments, R^{a}, R^{b} or R^{c} is substituted or unsubstituted —C₅₋₁₄ cycloalkenyl being a non-aromatic cyclic hydrocarbyl group having from 5 to 14 carbon atoms, and one or more double bonds. In some embodiments, R^{a}, R^{b} or R^{c} is —C₆₋₁₂ cycloalkenyl. In some embodiments, R^{a}, R^{b} or R^{c} is cyclooctenyl, cyclononenyl or cyclodecenyl. When R^{a}, R^{b} or R^{c} is substituted, R^{a}, R^{b} or R^{c} is substituted with one or more groups selected from Substituent Group A.

In some embodiments, R^{a}, R^{b} or R^{c} is substituted or unsubstituted —C₈₋₁₄ cycloalkynyl being a non-aromatic cyclic hydrocarbyl group having from 8 to 14 carbon atoms, and one or more triple bonds. In some embodiments, R^{a}, R^{b} or R^{c} is —C₈₋₁₂ cycloalkynyl. In some embodiments, R^{a}, R^{b} or R^{c} is cyclooctynyl, cyclononynyl or cyclodecynyl. When R^{a}, R^{b} or R^{c} is substituted, R^{a}, R^{b} or R^{c} is substituted with one or more groups selected from Substituent Group A.

When R^{a}, R^{b} and R^{c} are not ═O, each of the groups -L¹-R^{a}, -L²-R^{b} and -L³-R^{c} may be attached in the α or β configuration on the respective ring. That is, the group may be positioned 3α- or 3β-of the steroid group, or the group may be positioned 16α- or 16β-of the steroid group, or the group may be positioned 17α- or 17β-of the steroid group.

In some embodiments, R^{a}, R^{b} or R^{c} is an anti-parasite moiety. An anti-parasite moiety is a substituent formed from a compound with anti-parasite activity (e.g. a compound with anti-protozoan activity, such as anti-apicomplexan activity, or a compound with anti-helminth activity). In some embodiments, the anti-parasite moiety is an anti-protozoan moiety or an anti-helminth moiety. In some embodiments, R^{a}, R^{b} or R^{c} is an anti-protozoan moiety. In some embodiments, R^{a}, R^{b} or R^{c} is an anti-apicomplexan moiety. In some embodiments, R^{a}, R^{b} or R^{c} is an anti-apicomplexan moiety selected from:

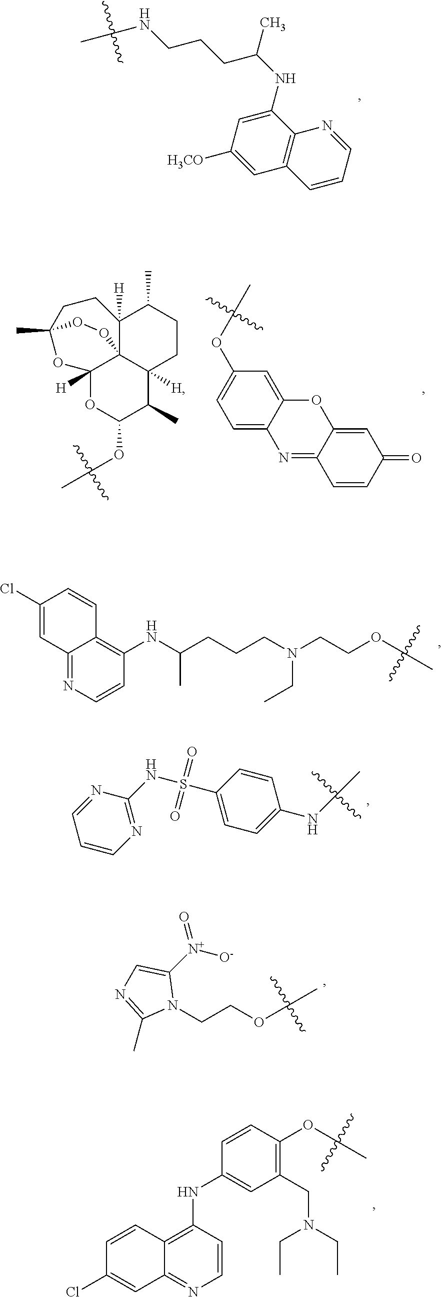

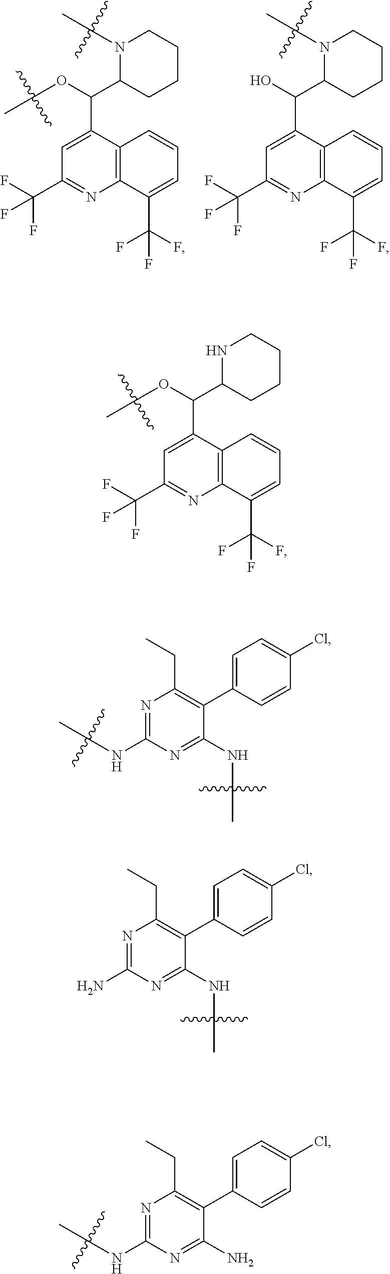

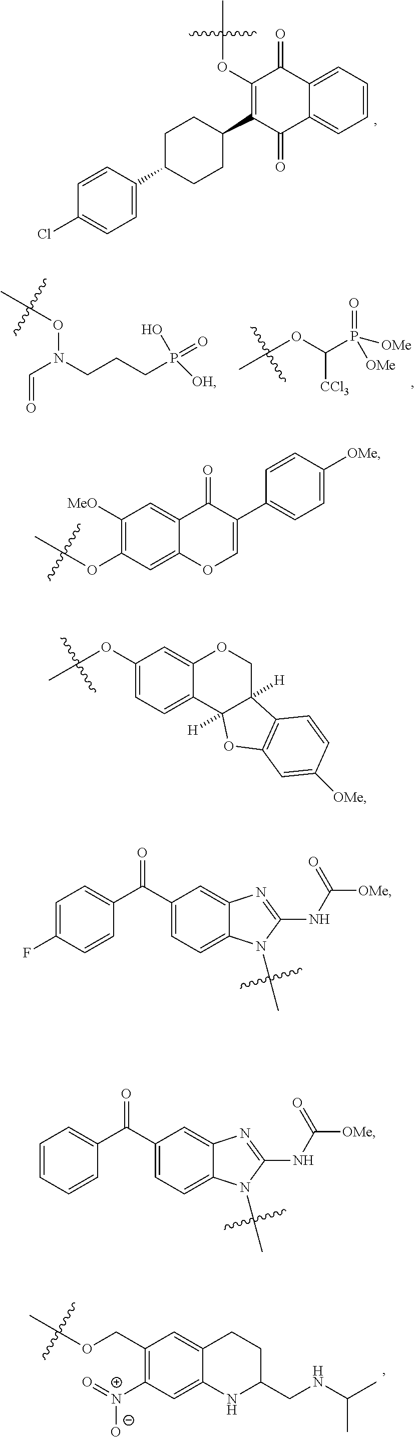

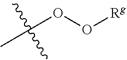

wherein R^{g} is H or substituted or unsubstituted —C₁₋₆ alkyl,

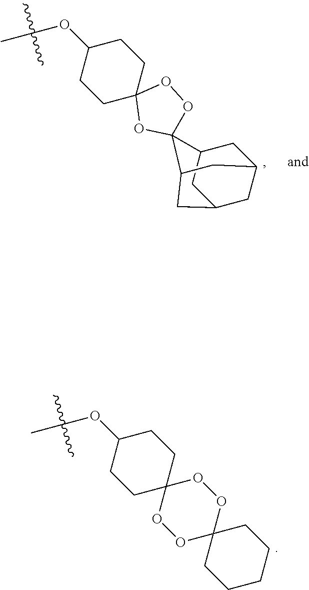 [SMILES: *OC1CCC2(CC1)OOC1(O2)C2CC3CC(C2)CC1C3 ; *OC1CCC2(CC1)OOC1(CCCCC1)OO2]

In some embodiments, R^{a}, R^{b} or R^{c} is

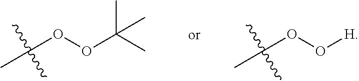

In some embodiments, when R^{a}, R^{b} or R^{c} is

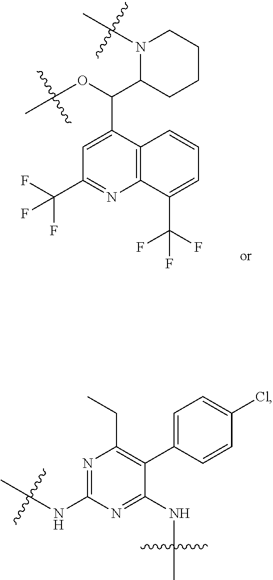 [SMILES: *OC(c1cc(C(F)(F)F)nc2c(C(F)(F)F)cccc12)C1CCCCN1* ; *Nc1nc(CC)c(-c2ccc(Cl)cc2)c(N*)n1]

there are two steroid groups attached at the positions shown.

In some embodiments, R^{a}, R^{b} or R^{c} is an anti-apicomplexan moiety selected from:

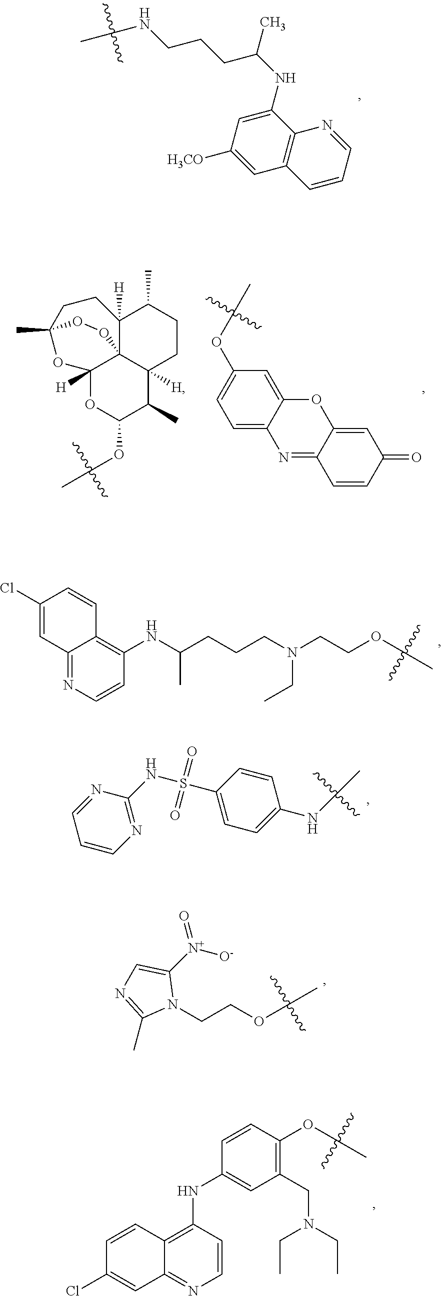

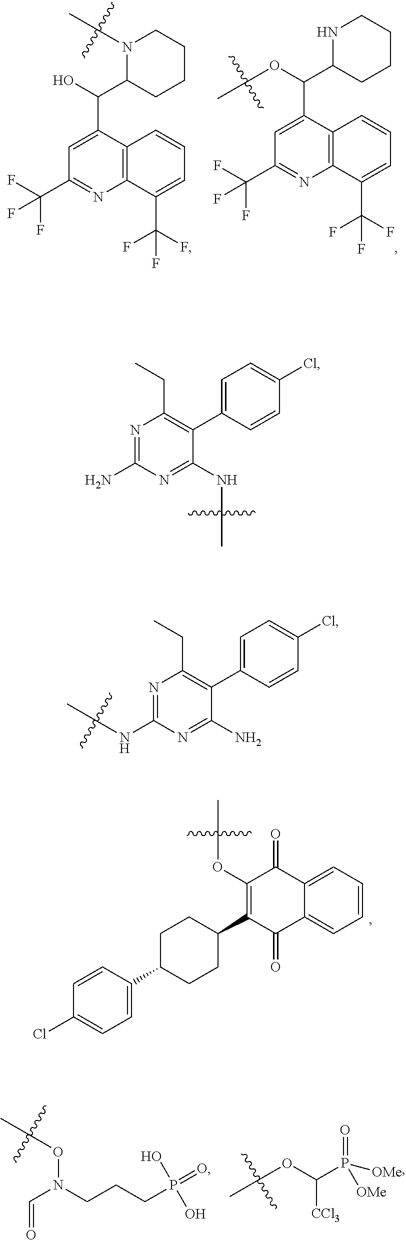

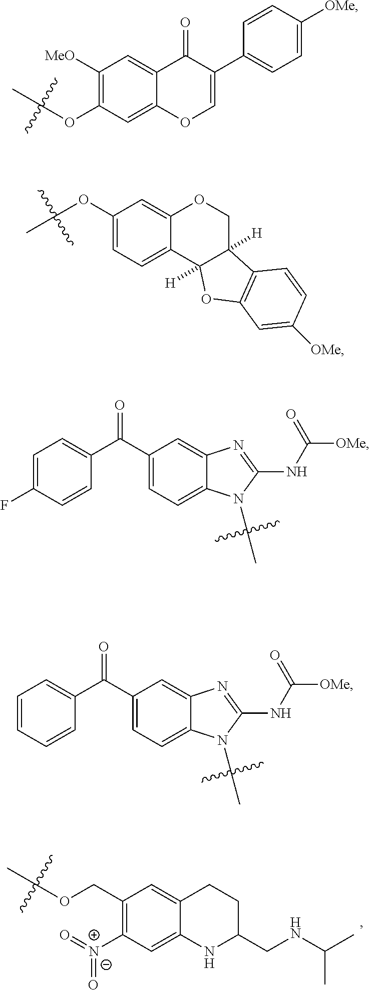

wherein R^{g} is H or substituted or unsubstituted —C₁₋₆ alkyl,

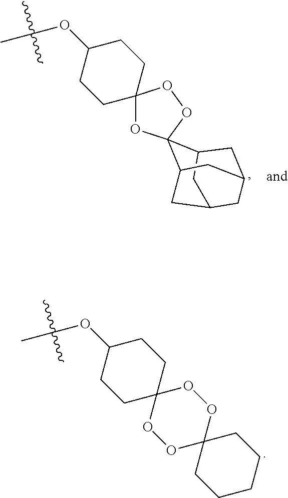 [SMILES: *OC1CCC2(CC1)OOC1(O2)C2CC3CC(C2)CC1C3 ; *OC1CCC2(CC1)OOC1(CCCCC1)OO2]

In Formula (I), at least one of R^{a}, R^{b} and R^{c} is an anti-parasite moiety.

In some embodiments, at least one of R^{a} and R^{c} is an anti-parasite moiety.

In some embodiments, at least one of R^{a} and R^{c} is an anti-parasite moiety, and R^{b} is not an anti-parasite moiety.

In some embodiments, R^{a} is an anti-parasite moiety. In some embodiments, R^{a} is an anti-parasite moiety, and one or both of R^{b} and R^{c} are not anti-parasite moieties. In some embodiments, R^{a} is an anti-parasite moiety, and both R^{b} and R^{c} are not anti-parasite moieties.

In some embodiments, R^{b} is an anti-parasite moiety. In some embodiments, R^{b} is an anti-parasite moiety, and one or both of R^{a} and R^{c} are not anti-parasite moieties. In some embodiments, R^{b} is an anti-parasite moiety, and both R^{a} and R^{c} are not anti-parasite moieties.

In some embodiments, R^{c} is an anti-parasite moiety. In some embodiments, R^{c} is an anti-parasite moiety, and one or both of R^{a} and R^{b} are not anti-parasite moieties. In some embodiments, R^{c} is an anti-parasite moiety, and both R^{a} and R^{b} are not anti-parasite moieties.

In some embodiments, R^{a} is not an anti-parasite moiety.

In some embodiments, R^{b} is not an anti-parasite moiety.

In some embodiments, R^{c} is not an anti-parasite moiety.

In some embodiments, each of R^{a} and R^{c} is independently selected from —H, —OH, ═O, substituted or unsubstituted —C₁₋₁₀ alkyl, substituted or unsubstituted —C₂₋₁₀ alkenyl, substituted or unsubstituted —C₂₋₁₀ alkynyl, substituted or unsubstituted —C₃₋₁₀ cycloalkyl, substituted or unsubstituted —C₈₋₁₄ cycloalkenyl, substituted or unsubstituted —C₈₋₁₄ cycloalkynyl, and an anti-parasite moiety; wherein at least one of R^{a} and R^{c} is an anti-parasite moiety.

In some embodiments, R^{b} is selected from —H, —OH, ═O, substituted or unsubstituted —C₁₋₁₀ alkyl, substituted or unsubstituted —C₂₋₁₀ alkenyl, substituted or unsubstituted —C₂₋₁₀ alkynyl, substituted or unsubstituted —C₃₋₁₀ cycloalkyl, substituted or unsubstituted —C₅₋₁₄ cycloalkenyl, and substituted or unsubstituted —C₈₋₁₄ cycloalkynyl.

In some embodiments, R^{d1} and R^{a} taken together form a ring selected from:

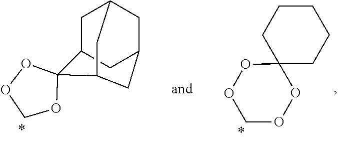

wherein the asterisk represents the point of attachment to the steroid structure (i.e. the asterisk represents C17 of the steroid structure when R^{d1} and R^{a} taken together form a ring with the C17 of the steroid structure).

In some embodiments, R^{d2} is present and is —H. In some embodiments, R^{d2} is absent.

In some embodiments, R^{d3} and R^{c} taken together form a ring selected from:

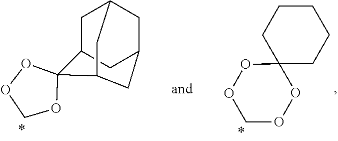

wherein the asterisk represents the point of attachment to the steroid structure (i.e. the asterisk represents C16 of the steroid structure when R^{d3} and R^{c} taken together form a ring with the C16 of the steroid structure).

In some embodiments, R^{e} is H. In some embodiments, R^{e} is CH₃.

In some embodiments, R^{e} is absent, and ring A is an aromatic ring.

In some embodiments, R^{e} is absent, and either C5-C10 or C1-C10 is a double bond. If C5-C10 is a double bond, then C5-C5 is a single bond.

In some embodiments, R^{e} is H, and ring A is a non-aromatic ring. In some embodiments, R^{e} is CH₃, and ring A is a non-aromatic ring.

In some embodiments, each of L¹, L² and L³, when present, may be substituted or unsubstituted and is independently selected from:

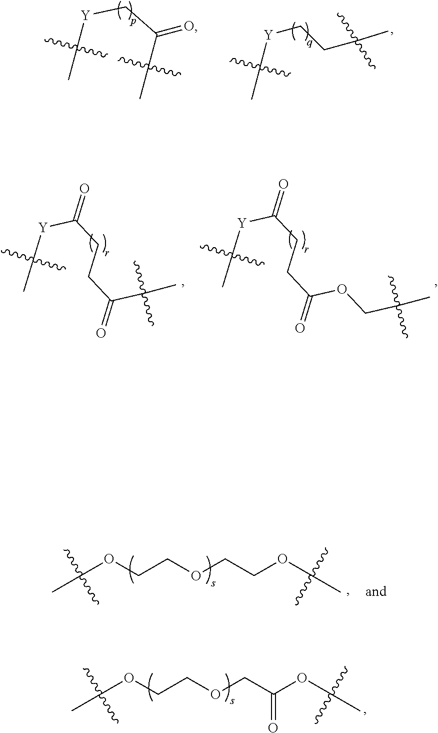

wherein Y is —NR^{f}—, —S—, —O— or —CR^{f1}R^{f2}—, wherein each of R^{a}, R^{f1} and R^{f2} is H or substituted or unsubstituted —C₁₋₆ alkyl; each of p, q, r and s is independently 0, 1, 2, 3 or 4; wherein, when L¹, L² or L³ is substituted, L¹. L² or L³ is substituted with one or more groups selected from Substituent Group A.

In some embodiments, each of L¹, L² and L³ may be substituted or unsubstituted and is independently selected from:

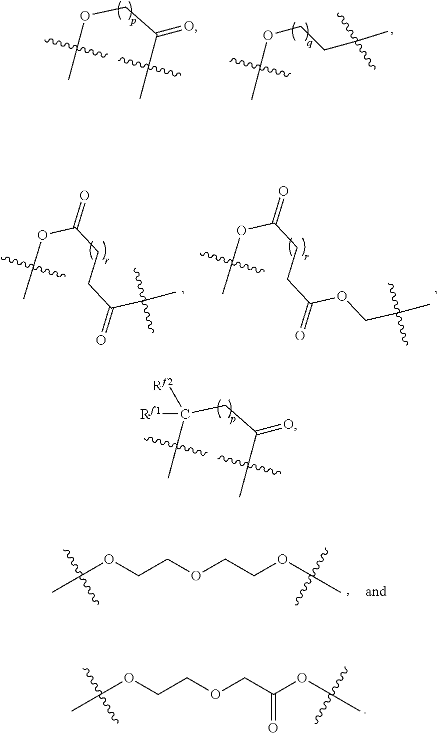

In some embodiments, each of L¹, L² and L³ may be substituted or unsubstituted and is independently selected from:

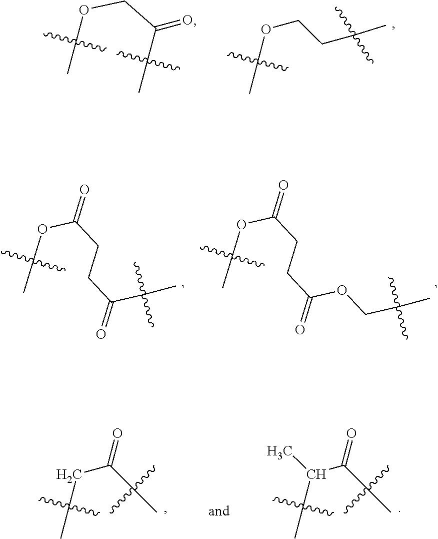

In one embodiment, the steroid-active conjugate of Formula (I) is a compound of Formula (Ia):

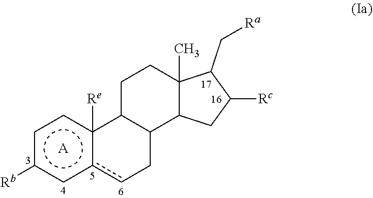

wherein:
R^{a} is selected from:

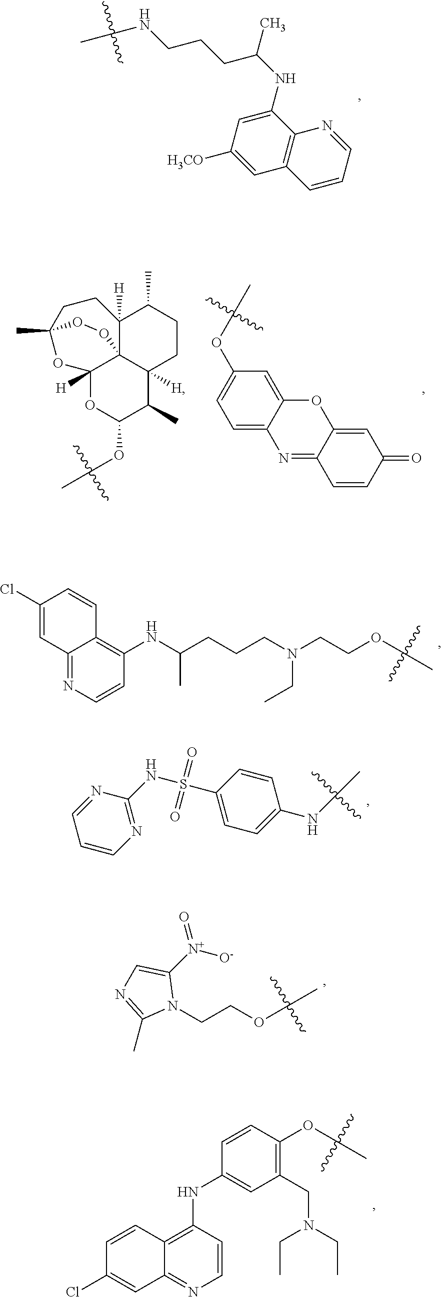

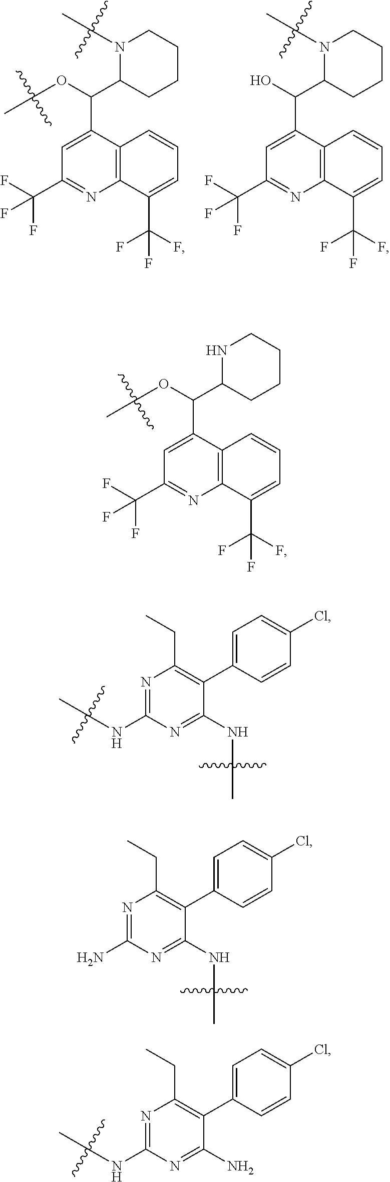

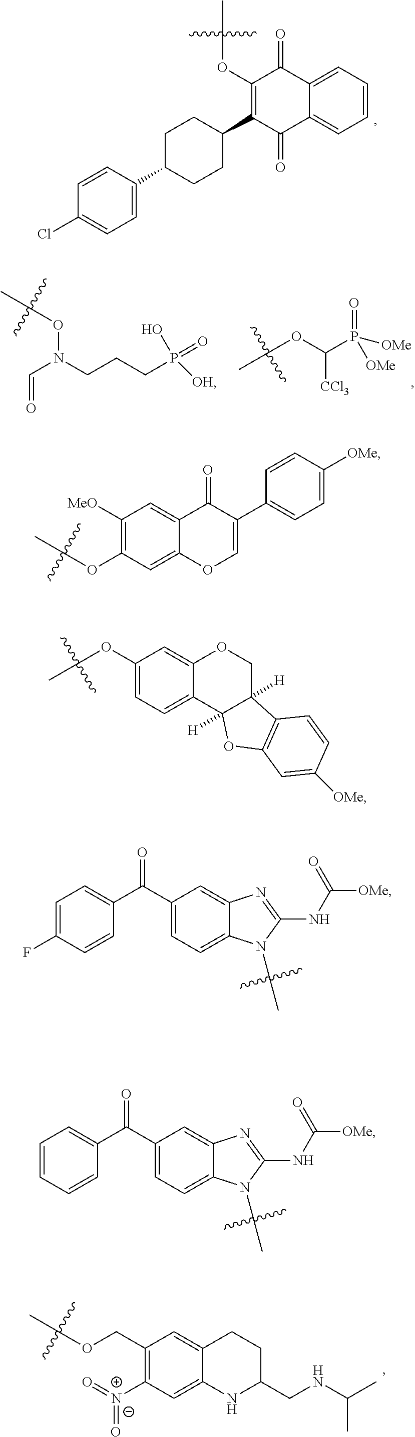

wherein R^{g} is H or substituted or unsubstituted —C₁₋₆ alkyl,

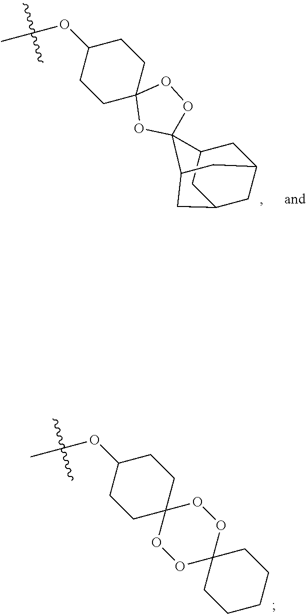 [SMILES: *OC1CCC2(CC1)OOC1(O2)C2CC3CC(C2)CC1C3 ; *OC1CCC2(CC1)OOC1(CCCCC1)OO2]

each of R^{b} and R^{c} is selected from —H, —OH, ═O, substituted or unsubstituted —C₁₋₁₀ alkyl, substituted or unsubstituted —C₂₋₁₀ alkenyl, substituted or unsubstituted —C₂₋₁₀ alkynyl, substituted or unsubstituted —C₃₋₁₀ cycloalkyl; substituted or unsubstituted —C₅₋₁₄ cycloalkenyl, or substituted or unsubstituted —C₈₋₁₄ cycloalkynyl, wherein, when R^{b} or R^{c} is substituted. R^{b} or R^{c} is substituted with one or more groups selected from Substituent Group A;
R^{e} is H or CH₃, or R^{e} is absent;
L¹ may be substituted or unsubstituted and is independently selected from:

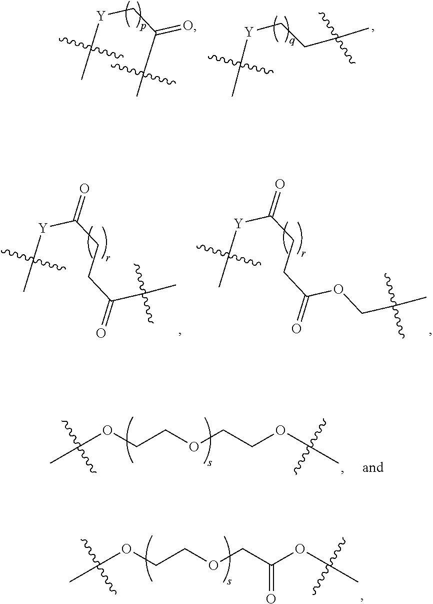

wherein Y is —NR^{f}—, —S—, —O— or —CR^{f1}R^{f2}—, wherein each of R^{f}, R^{f1} and R^{f2} is H or substituted or unsubstituted —C₁₋₆ alkyl; each of p, q, r and s is independently 0, 1, 2, 3 or 4; wherein, when L¹ is substituted, L¹ is substituted with one or more groups selected from Substituent Group A;
ring A may be a saturated or unsaturated ring, or ring A may be an aromatic ring, provided that when ring A is an aromatic ring, the C5-C6 bond is a single bond, R^{e} is absent, and R^{b} is not ═O;
[symbol] is a single bond or a double bond; provided that when the C4-C5 or C5-C10 bond is a double bond, the C5-C6 bond is a single bond; when the C5-C6 bond is a double bond, the C4-C5 and C5-C10 bonds are single bonds; and when the C3 forms a double bond with C2 or C4, R^{b} is not ═O;
or a stereoisomer thereof, or a salt thereof.

In one embodiment, the steroid-active conjugate of Formula (I) is a compound of Formula (Ib):

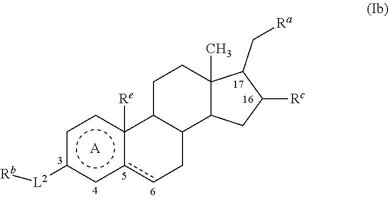

wherein:
each of R^{a} and R^{c} is selected from —H, —OH, ═O, substituted or unsubstituted —C₁₋₁₀ alkyl, substituted or unsubstituted —C₂₋₁₀ alkenyl, substituted or unsubstituted —C₂₋₁₀ alkynyl, substituted or unsubstituted —C₃₋₁₀ cycloalkyl, substituted or unsubstituted —C₅₋₁₄ cycloalkenyl, or substituted or unsubstituted —C₈₋₁₄ cycloalkynyl, wherein, when R^{a} or R^{c} is substituted, R^{a} or R^{c} is substituted with one or more groups selected from Substituent Group A;
R^{b} is selected from:

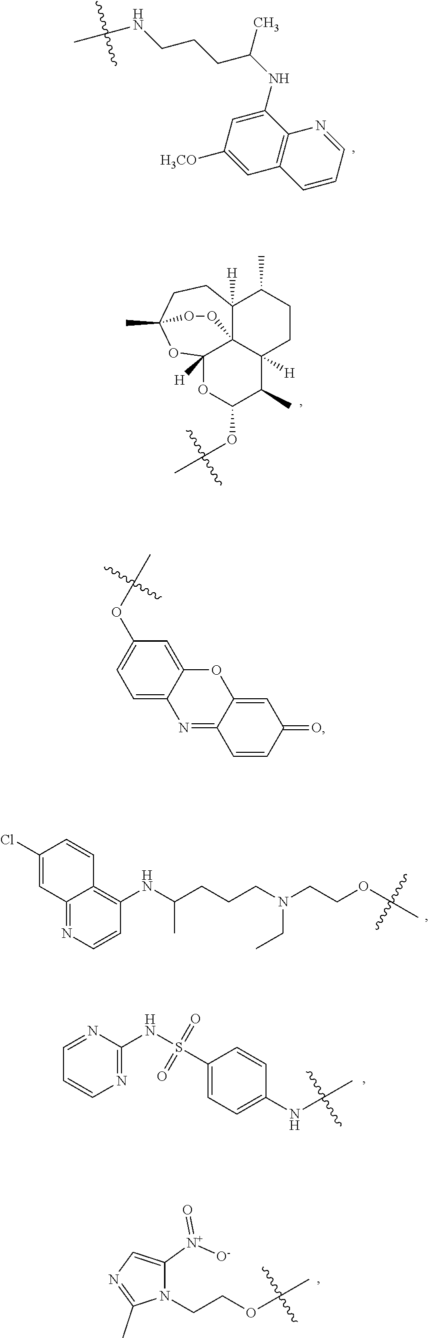

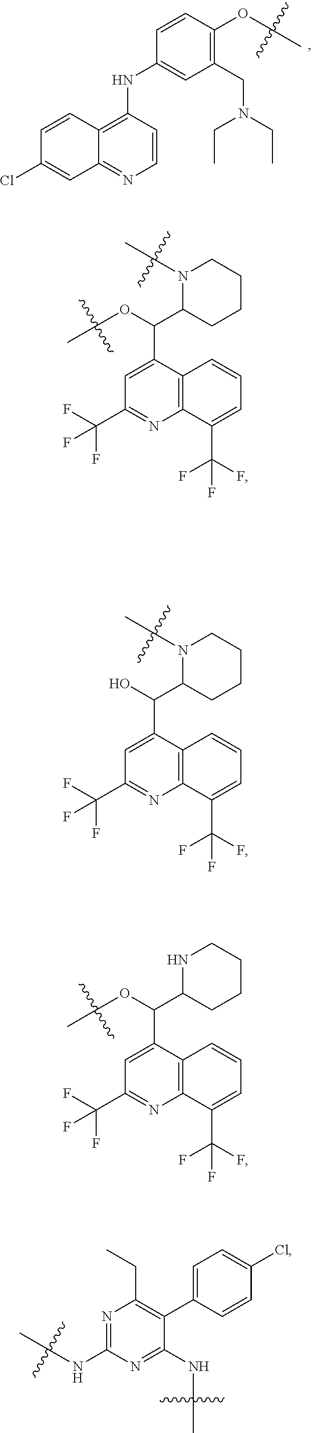

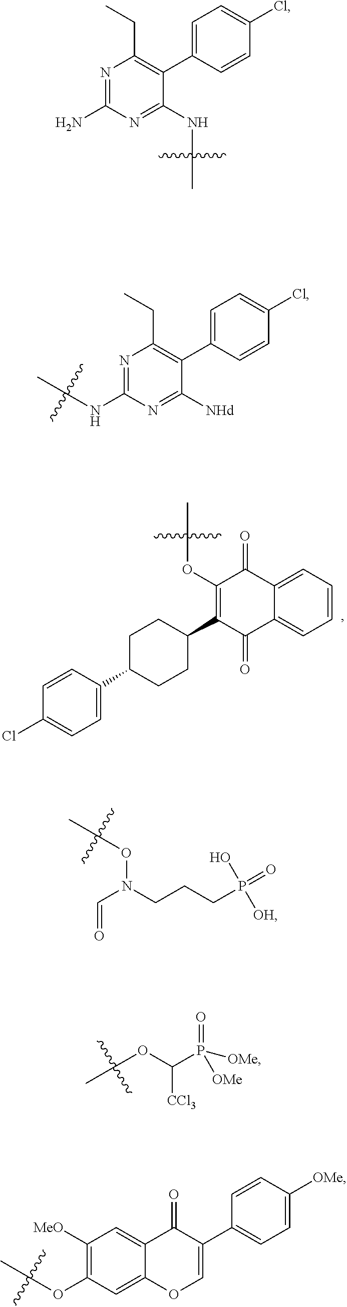

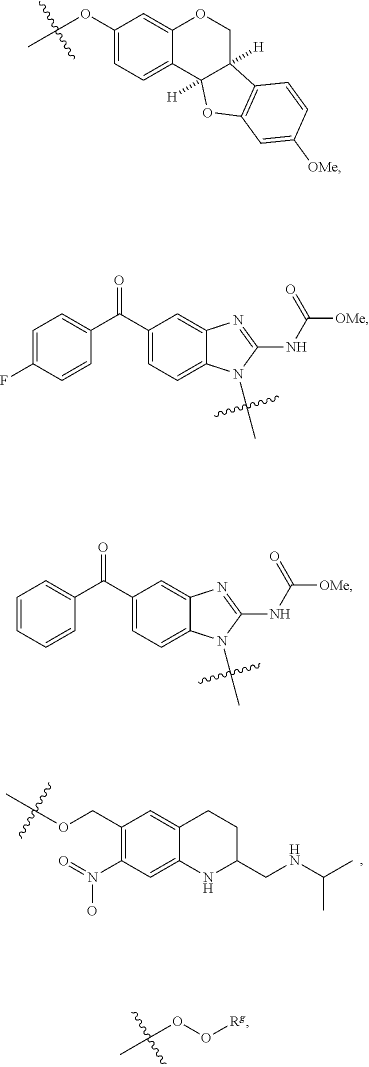

wherein R^{g} is H or substituted or unsubstituted —C₁₋₆ alkyl,

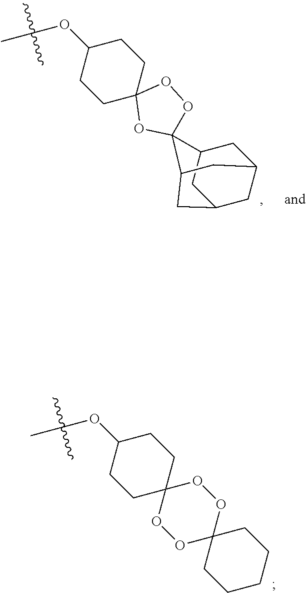 [SMILES: *OC1CCC2(CC1)OOC1(O2)C2CC3CC(C2)CC1C3 ; *OC1CCC2(CC1)OOC1(CCCCC1)OO2]

R^{e} is H or CH₃, or R^{e} is absent;
L² may be substituted or unsubstituted and is independently selected from:

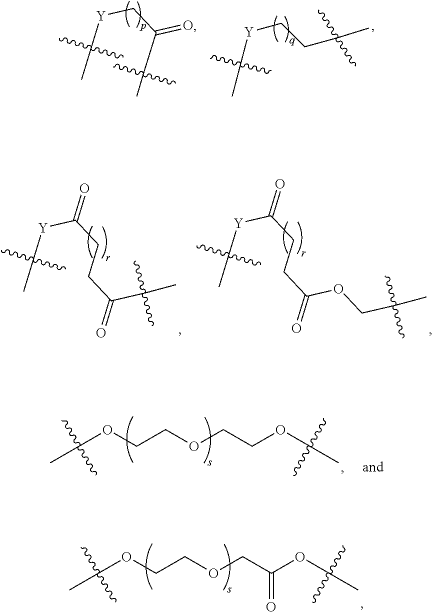

wherein Y is —NR^{f}—, —S—, —O— or —CR^{f1}R^{f2}—, wherein each of R^{f}, R^{f1} and R^{f2} is H or substituted or unsubstituted —C₁₋₆ alkyl; each of p, q, r and s is independently 0, 1, 2, 3 or 4; wherein, when L² is substituted, L² is substituted with one or more groups selected from Substituent Group A;
ring A may be a saturated or unsaturated ring, or ring A may be an aromatic ring, provided that when ring A is an aromatic ring, the C5-C6 bond is a single bond. R^{e} is absent, and R^{b} is not ═O;
[symbol] is a single bond or a double bond; provided that when the C4-C5 or C5-C10 bond is a double bond, the C5-C6 bond is a single bond; when the C5-C6 bond is a double bond, the C4-C5 and C5-C10 bonds are single bonds; and when the C3 forms a double bond with C2 or C4, R^{b} is not ═O;
or a stereoisomer thereof, or a salt thereof.

In one embodiment, the steroid-active conjugate of Formula (I) is a compound of Formula (Ic):

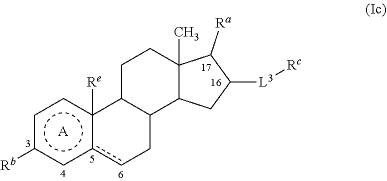

wherein:
each of R^{a} and R^{b} is selected from —H, —OH, ═O, substituted or unsubstituted —C₁₋₁₀ alkyl, substituted or unsubstituted —C₂₋₁₀ alkenyl, substituted or unsubstituted —C₂₋₁₀ alkynyl, substituted or unsubstituted —C₃₋₁₀ cycloalkyl; substituted or unsubstituted —C₅₋₁₄ cycloalkenyl, or substituted or unsubstituted —C₈₋₁₄ cycloalkynyl, wherein, when R^{a} or R^{b} is substituted, R^{a} or R^{b} is substituted with one or more groups selected from Substituent Group A;
R^{c} is selected from:

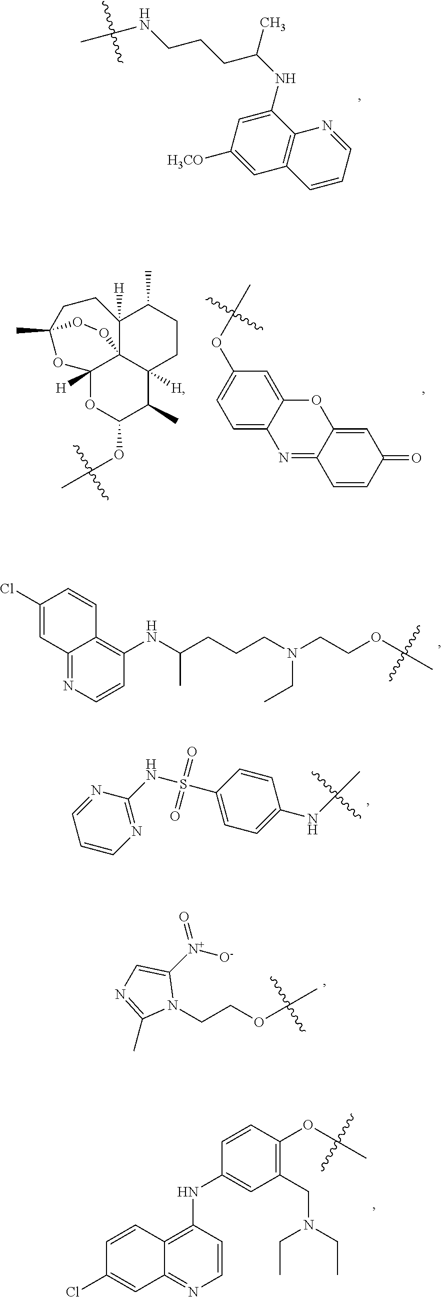

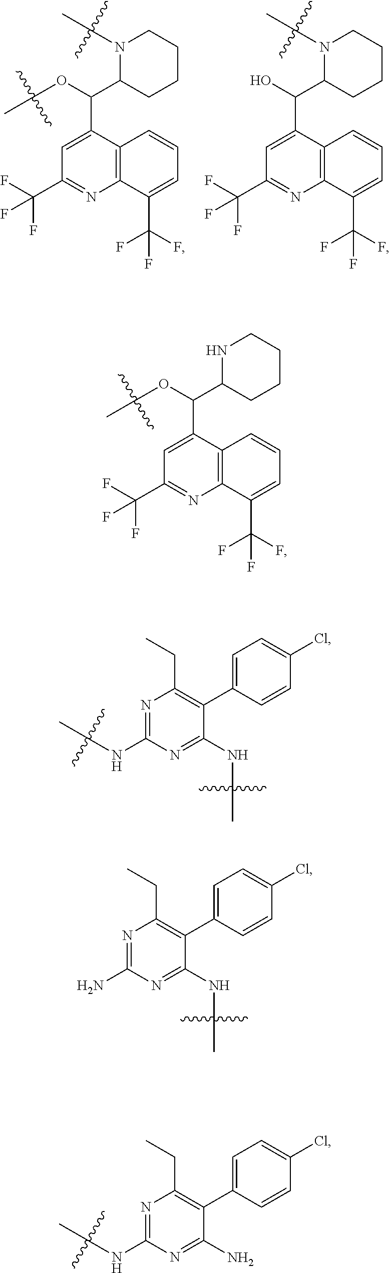

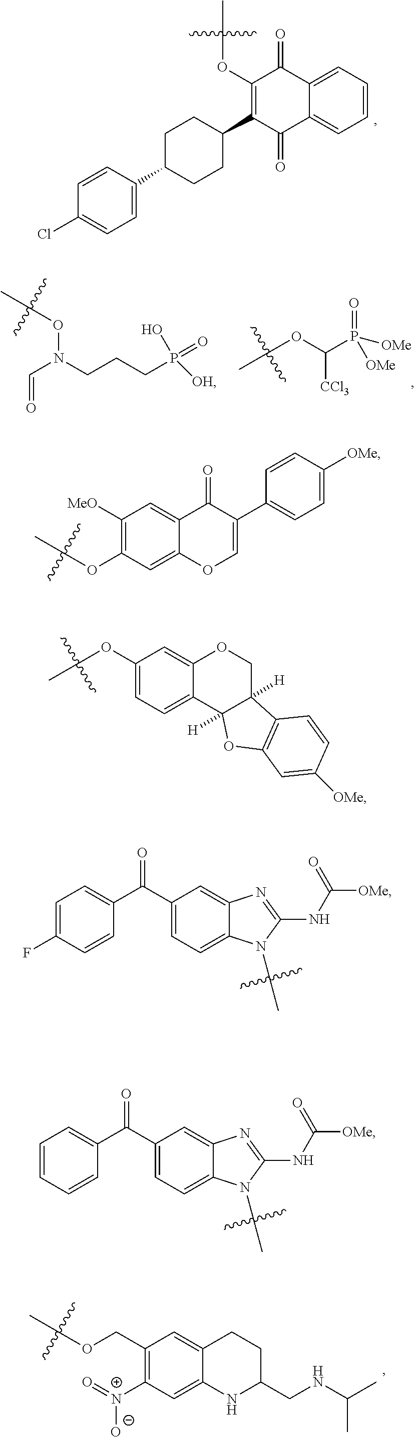

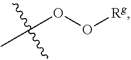

wherein R^{g} is H or substituted or unsubstituted —C₁₋₆ alkyl,

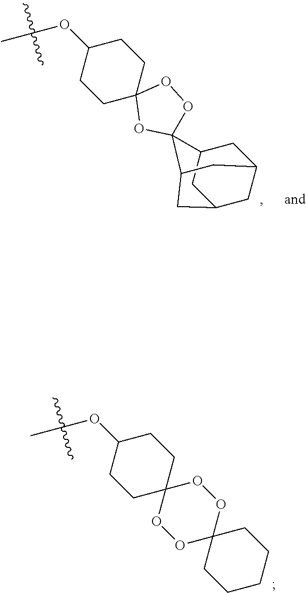 [SMILES: *OC1CCC2(CC1)OOC1(O2)C2CC3CC(C2)CC1C3 ; *OC1CCC2(CC1)OOC1(CCCCC1)OO2]

R^{e} is H or CH₃, or R^{e} is absent;
L³ may be substituted or unsubstituted and is independently selected from:

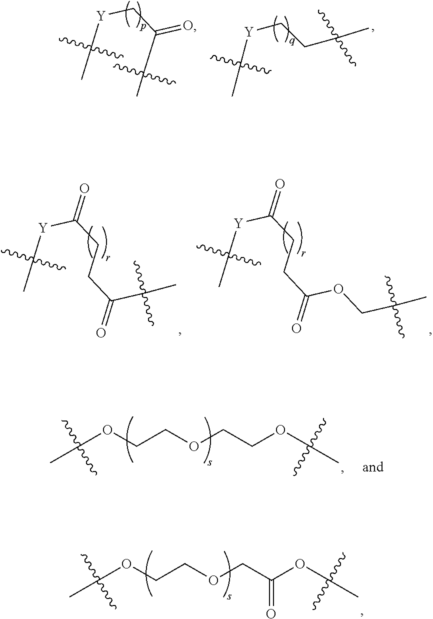

wherein Y is —NR^{f}—, —S—, —O— or —CR^{f1}R^{f2}—, wherein each of R^{f}, R^{f1} and R^{f2} is H or substituted or unsubstituted —C₁₋₆ alkyl; each of p, q, r and s is independently 0, 1, 2, 3 or 4; wherein, when L³ is substituted, L³ is substituted with one or more groups selected from Substituent Group A;
ring A may be a saturated or unsaturated ring, or ring A may be an aromatic ring, provided that when ring A is an aromatic ring, the C5-C6 bond is a single bond, R^{e} is absent, and R^{b} is not ═O;
[symbol] is a single bond or a double bond; provided that when the C4-C5 or C5-C10 bond is a double bond, the C5-C6 bond is a single bond; when the C5-C6 bond is a double bond, the C4-C5 and C5-C10 bonds are single bonds; and when the C3 forms a double bond with C2 or C4, R^{b} is not ═O;
or a stereoisomer thereof, or a salt thereof.

In some embodiments, Formula (Ia), Formula (Ib) or Formula (Ic) comprises a steroid group selected from:

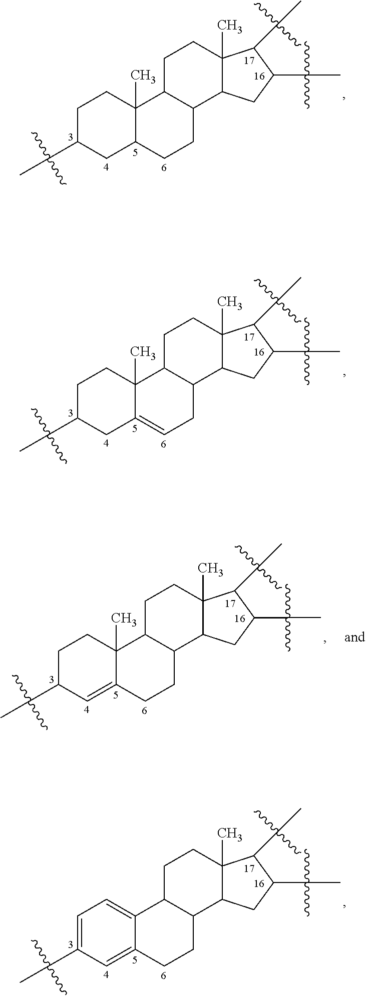 [SMILES: *C1CCC2(C)C(CCC3C2CCC2(C)C(*)C(*)CC32)C1 ; *C1CCC2(C)C(=CCC3C2CCC2(C)C(*)C(*)CC32)C1 ; *C1C=C2CCC3C(CCC4(C)C(*)C(*)CC34)C2(C)CC1 ; *c1ccc2c(c1)CCC1C2CCC2(C)C(*)C(*)CC12]

wherein the groups at C3, C16 and C17 may be attached in the α or β configuration on the respective ring. That is, the groups may be positioned 3α- or 3β-of the steroid group, or the group may be positioned 16α- or 16β-of the steroid group, or the group may be positioned 17α- or 17β-of the steroid group.

In some embodiments, each of L¹, L² and L³ of Formula (Ia), Formula (Ib) or Formula (Ic) is independently selected from:

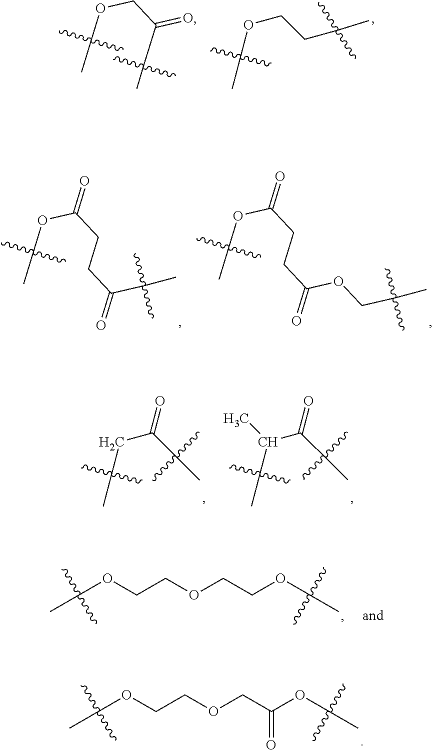

In some embodiments, the anti-parasite moiety R^{a}, R^{b} or R^{c} of Formula (Ia), Formula (Ib) or Formula (Ic) is selected from:

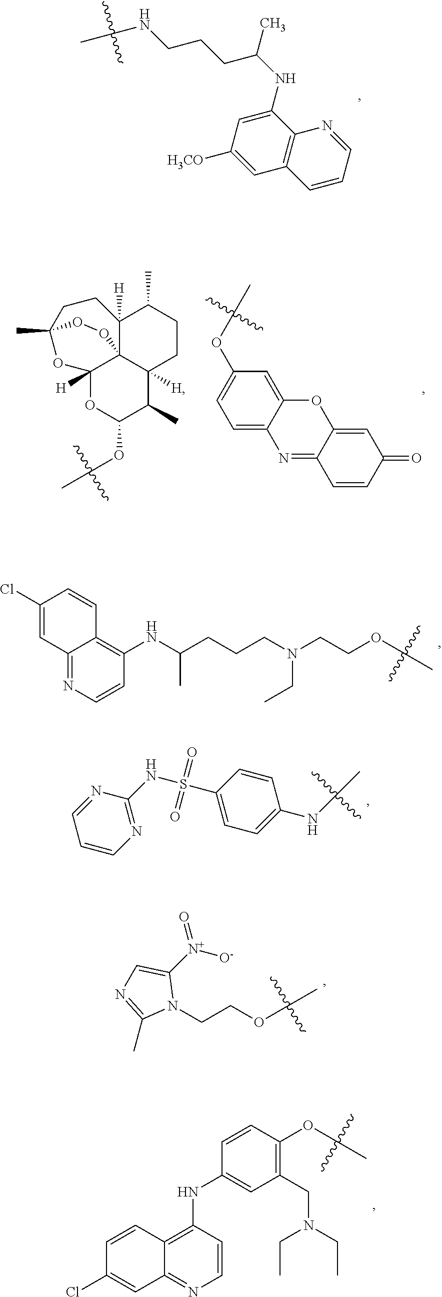

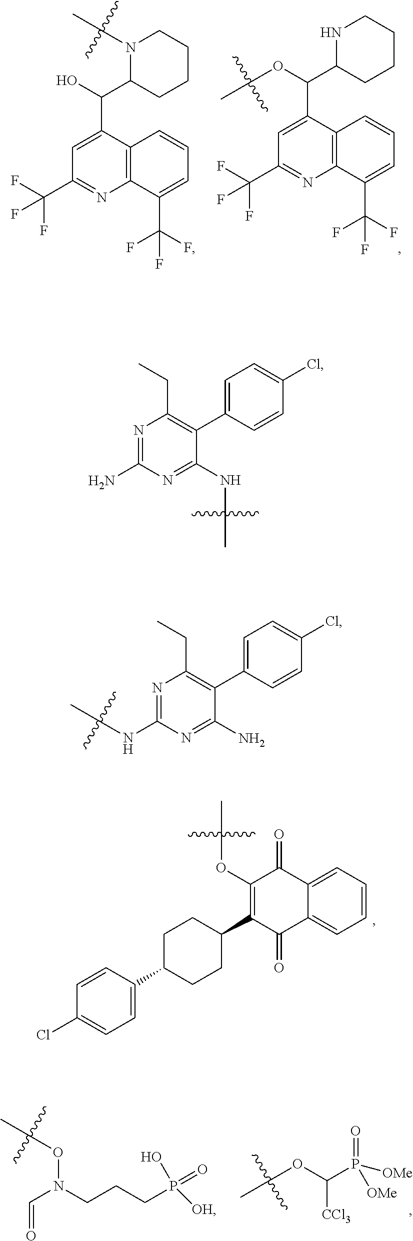

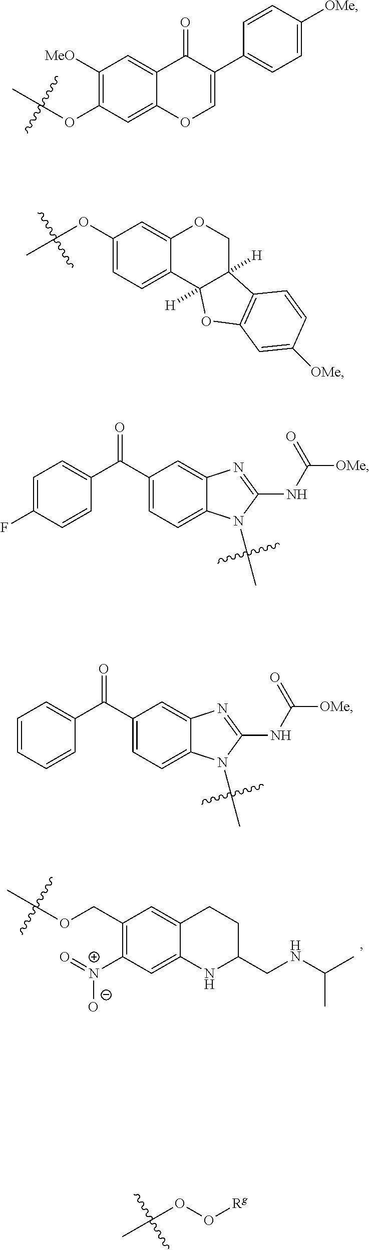

wherein R^{g} is H or substituted or unsubstituted —C₁₋₆ alkyl,

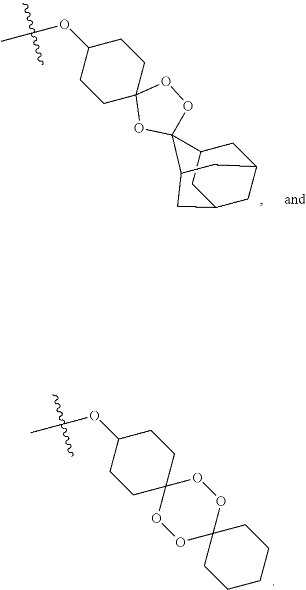 [SMILES: *OC1CCC2(CC1)OOC1(O2)C2CC3CC(C2)CC1C3 ; *OC1CCC2(CC1)OOC1(CCCCC1)OO2]

In some embodiments, R^{a}, R^{b} or R^{c} of Formula (Ia), Formula (Ib) or Formula (Ic) is selected from:

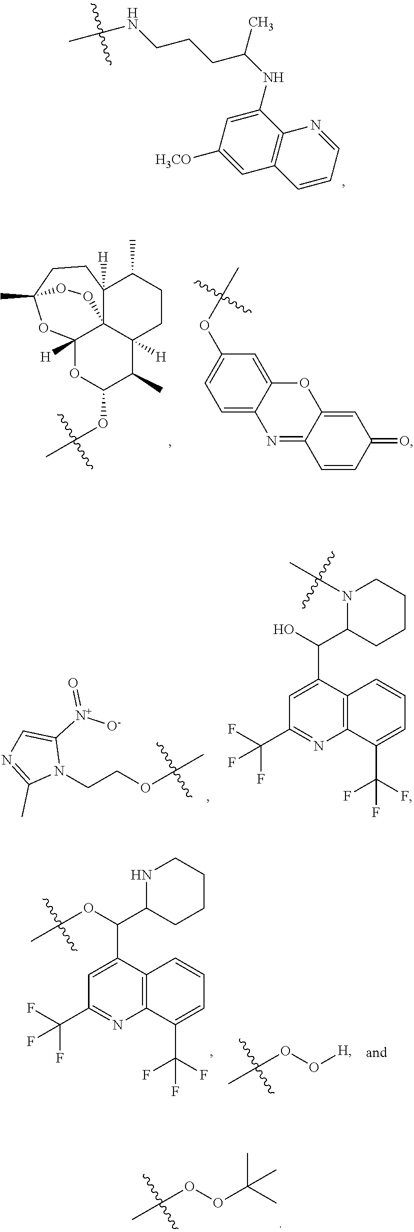

In some embodiments, R^{a}, R^{b} or R^{c} of Formula (Ia), Formula (Ib) or Formula (Ic) is selected from:

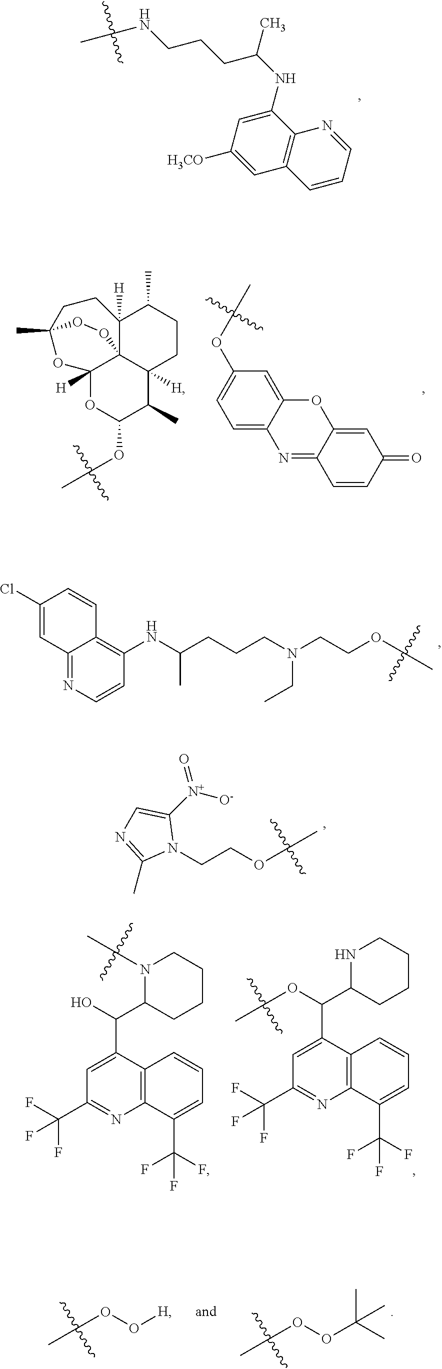

In some embodiments, the compound of Formula (I) is a compound of Formula (Ia) comprising a steroid group selected from:

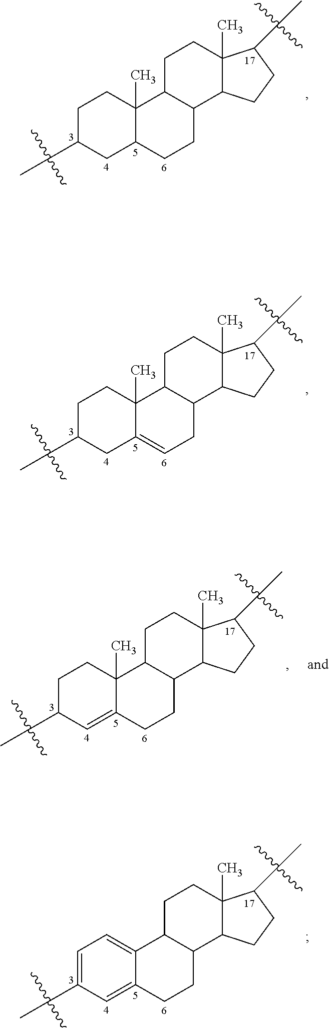

wherein:
L¹ is selected from:

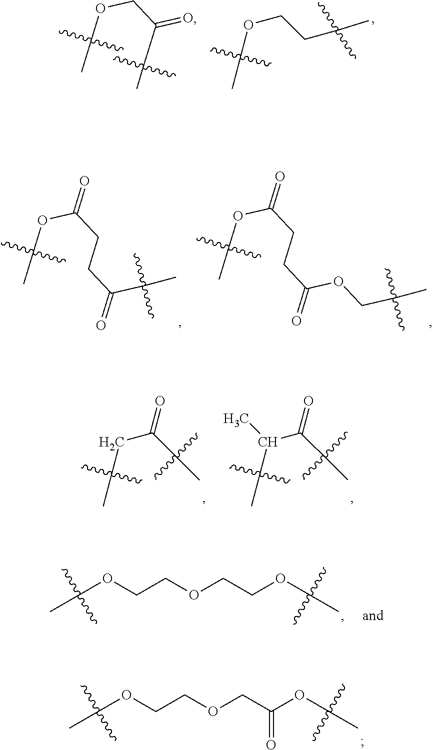

R^{a} is selected from:

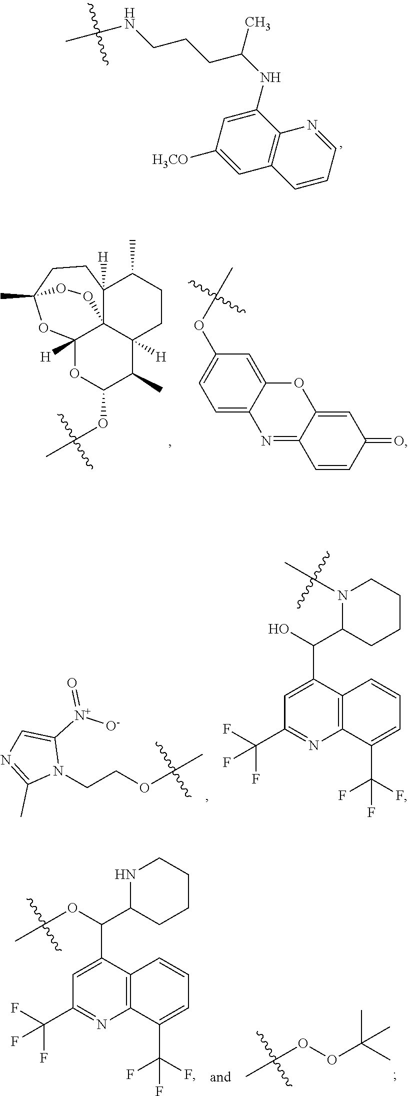

R^{b} is —OH.

In some embodiments, the compound of Formula (I) is a compound of Formula (Ia) comprising a steroid group selected from:

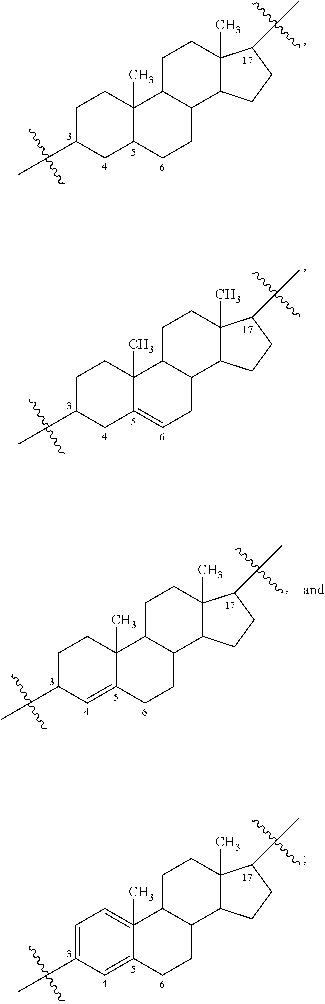

wherein:
L¹ is selected from:

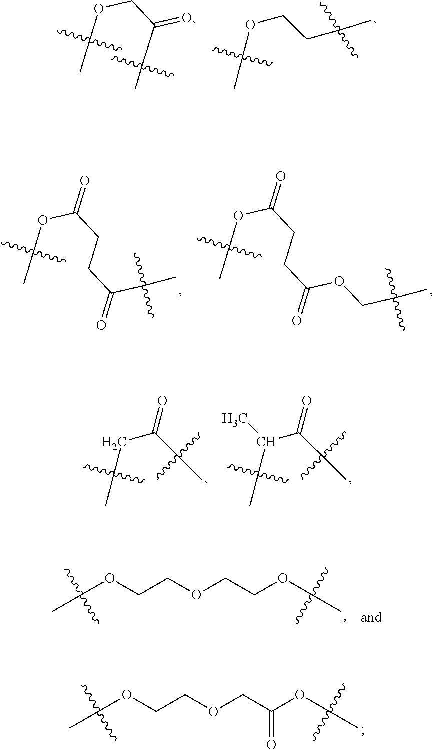

R^{a} is selected from:

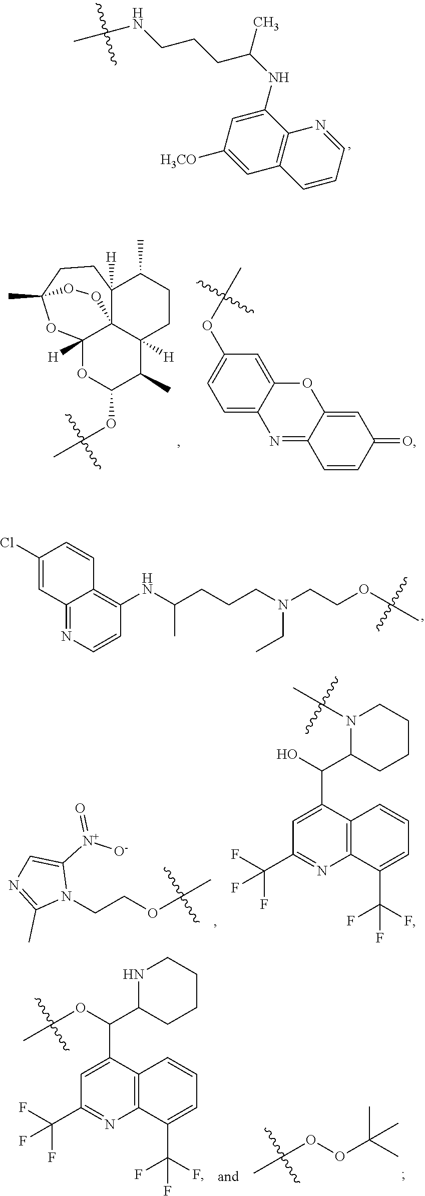

and
R^{b} is —OH.

In some embodiments, the compound of Formula (Ia) is a compound selected from any one of the following:

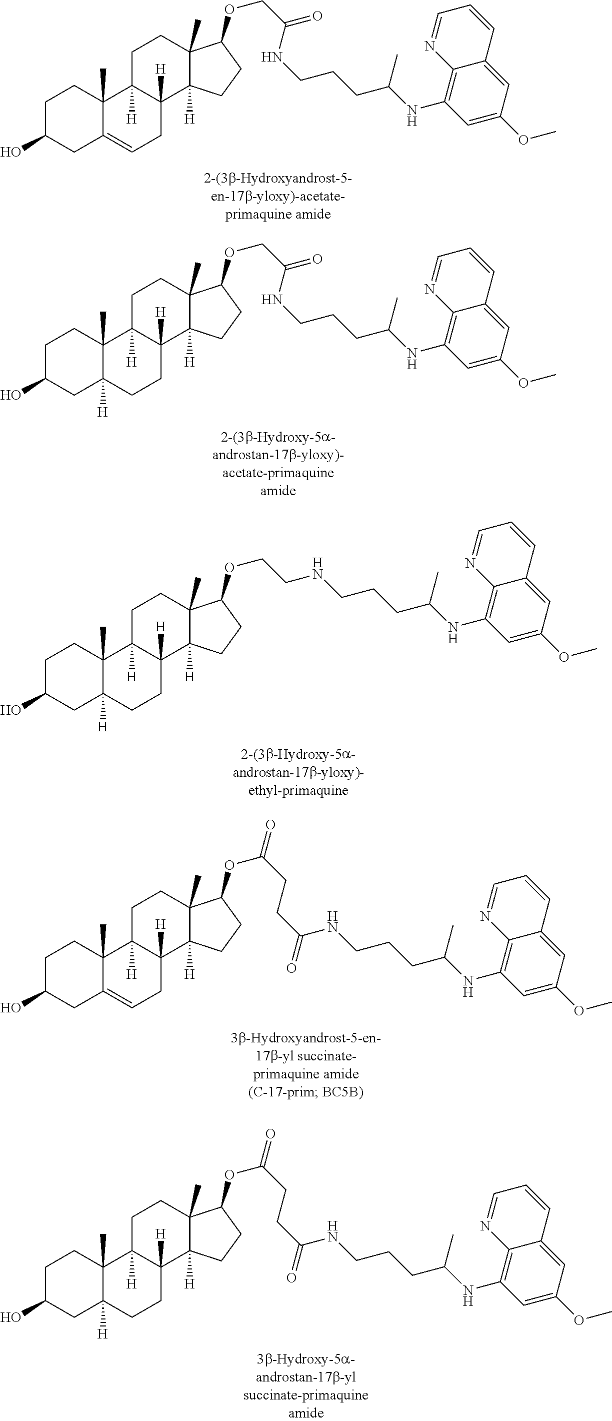

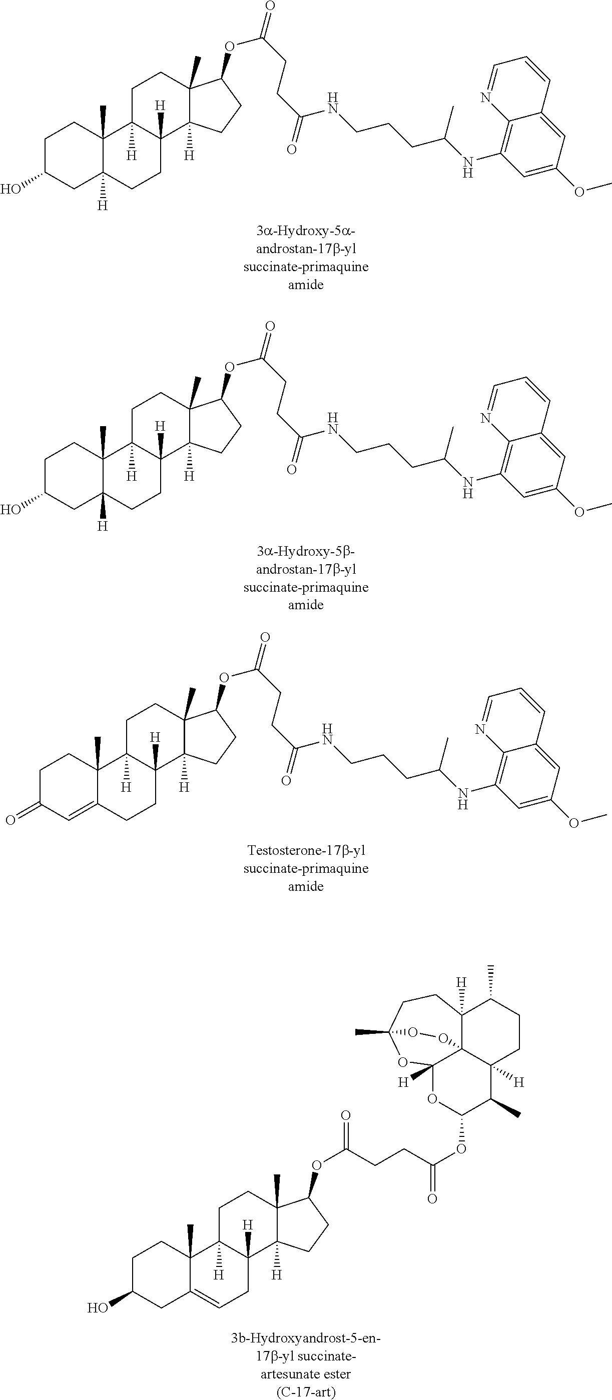

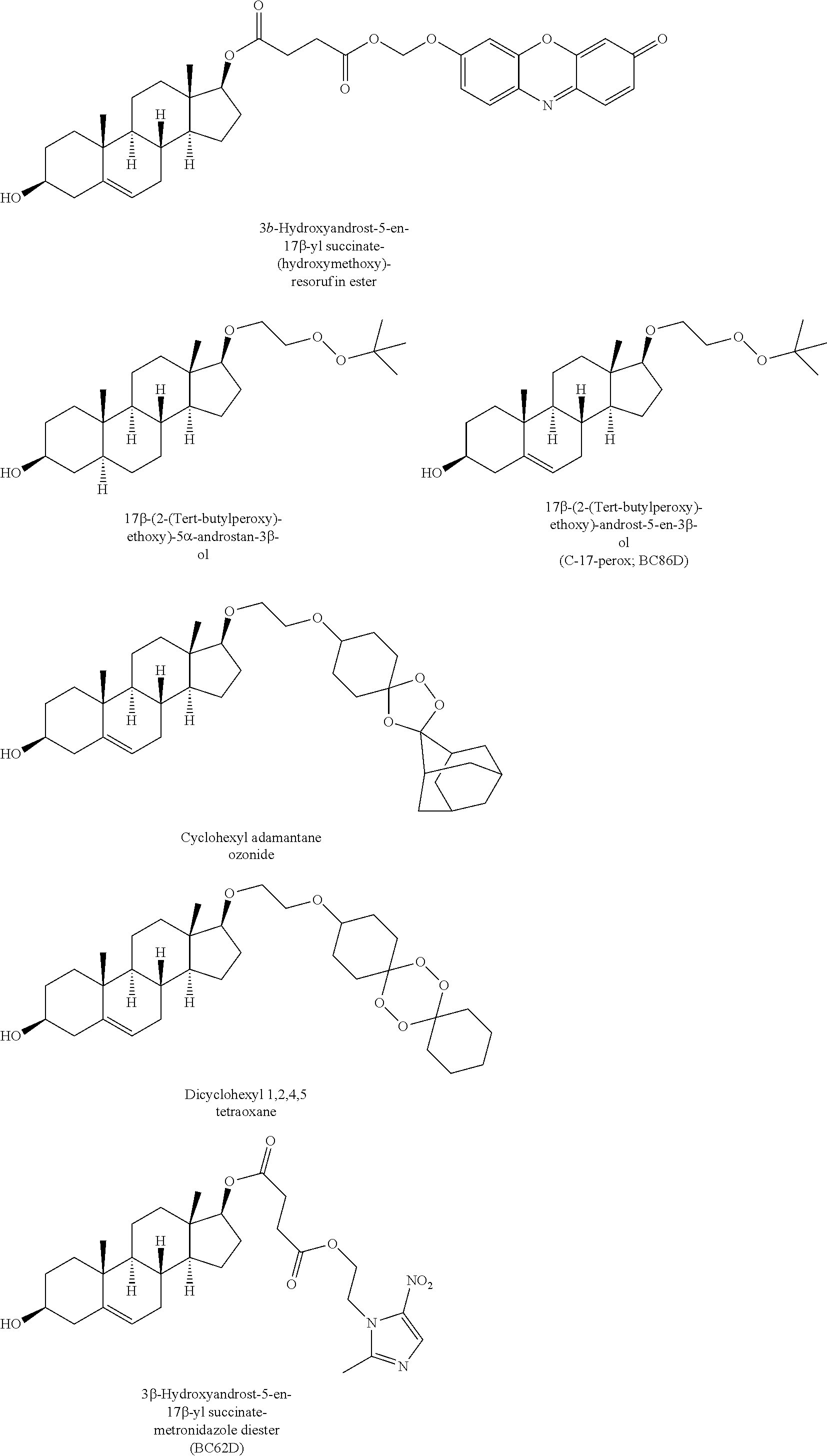

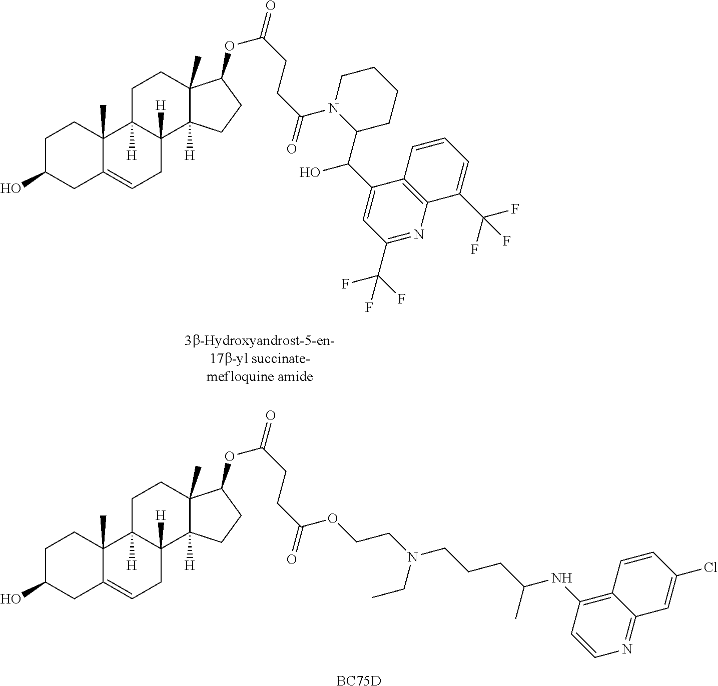

In some embodiments, the compound of Formula (Ib) is a compound selected from the following:

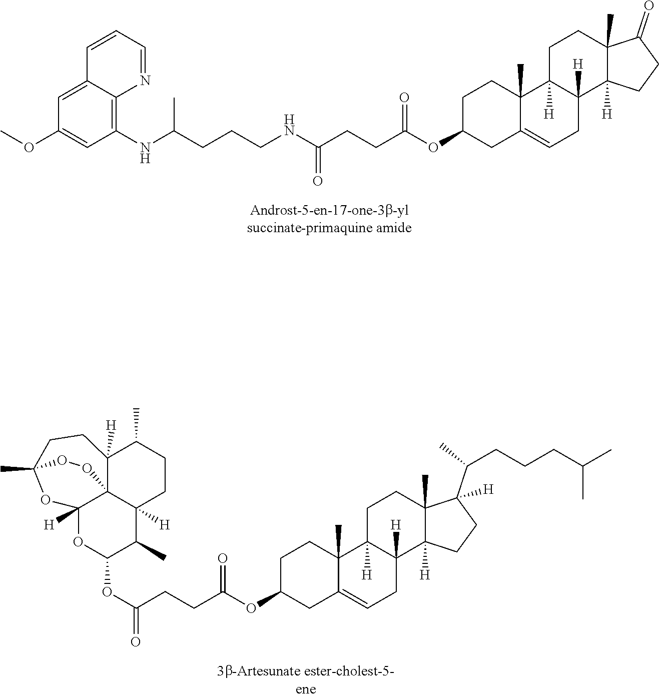

In some embodiments, the compound of Formula (I) is a compound selected from the following:

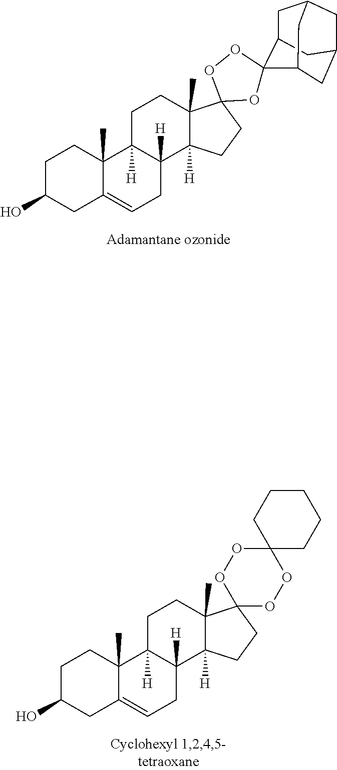

In some embodiments, the compound of Formula (I) is a compound of Formula (I″):

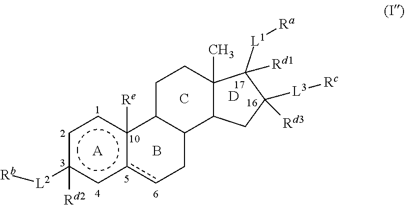

wherein:
each of R^{a} and R^{c} is independently selected from —H, —OH, ═O, substituted or unsubstituted —C₁₋₁₀ alkyl, substituted or unsubstituted —C₂₋₁₀ alkenyl, substituted or unsubstituted —C₂₋₁₀ alkynyl, substituted or unsubstituted —C₃₋₁₀ cycloalkyl, substituted or unsubstituted —C₅₋₁₄ cycloalkenyl, substituted or unsubstituted —C₈₋₁₄ cycloalkynyl, and an anti-parasite moiety; wherein at least one of R^{a} and R^{c} is an anti-parasite moiety;
R^{b} is selected from —H, —OH, ═O, substituted or unsubstituted —C₁₋₁₀ alkyl, substituted or unsubstituted —C₂₋₁₀ alkenyl, substituted or unsubstituted —C₂₋₁₀ alkynyl, substituted or unsubstituted —C₃₋₁₀ cycloalkyl, substituted or unsubstituted —C₅₋₁₄ cycloalkenyl, and substituted or unsubstituted —C₈₋₁₄ cycloalkynyl;
provided that: when R^{a} is —OH, L¹ is absent; when R^{a} is ═O, L¹ and R^{d1} are absent; when R^{b} is —OH, L² is absent; when R^{b} is ═O, L² is absent, R^{d2} is absent, and the C2-C3 bond and the C3-C4 bond are single bonds; when R^{c} is —OH, L³ is absent; when R^{c} is ═O, L³ and R^{d3} are absent;
when R^{a}, R^{b} or R^{c} is substituted, R^{a}, R^{b} or R^{c} is substituted with one or more groups selected from Substituent Group A;
R^{d1}, when present, is H or is a group that, together with R^{a}, forms a substituted or unsubstituted ozonide ring or a substituted or unsubstituted tetraoxane ring, wherein the optional substituent may be a —C₃₋₁₀ cycloalkyl; provided that when R^{d1} and R^{a} form a substituted or unsubstituted ozonide ring or a substituted or unsubstituted tetraoxane ring, L¹ is absent; wherein, when the ozonide or tetraoxane ring is substituted, the ozonide or tetraoxane ring is substituted with one or more —C₃₋₁₀ cycloalkyl groups;
R^{d2}, when present, is H; provided that when R^{d2} is present, the C2-C3 bond and the C3-C4 bond are single bonds;
R^{d3}, when present, is H or is a group that, together with R^{c}, forms a substituted or unsubstituted ozonide ring or a substituted or unsubstituted tetraoxane ring, wherein the optional substituent may be a —C₃₋₁₀ cycloalkyl; provided that when R^{d3} and R^{c} form a substituted or unsubstituted ozonide ring or a substituted or unsubstituted tetraoxane ring, L³ is absent; wherein, when the ozonide or tetraoxane ring is substituted, the ozonide or tetraoxane ring is substituted with one or more —C₃₋₁₀ cycloalkyl groups;
R^{e} is H or CH₃, or R^{e} is absent;
when present, L¹ is a group that provides a covalent linkage between R^{a} and the C17 of ring D;
when present, L² is a group that provides a covalent linkage between R^{b} and the C3 of ring A;
when present, L³ is a group that provides a covalent linkage between R^{c} and the C16 of ring D;
ring A may be a saturated or unsaturated ring, or ring A may be an aromatic ring, provided that when ring A is an aromatic ring, the C5-C6 bond is a single bond, R^{d2} is absent, R^{e} is absent, and R^{b} is not ═O;
[symbol] is a single bond or a double bond; provided that when the C4-C5 or C5-C10 bond is a double bond, the C5-C6 bond is a single bond; when the C5-C6 bond is a double bond, the C4-C5 and C5-C10 bonds are single bonds; and when the C3 forms a double bond with C2 or C4, R^{b} is not ═O;
or a stereoisomer thereof, or a salt thereof;
wherein the anti-parasite moiety is selected from:

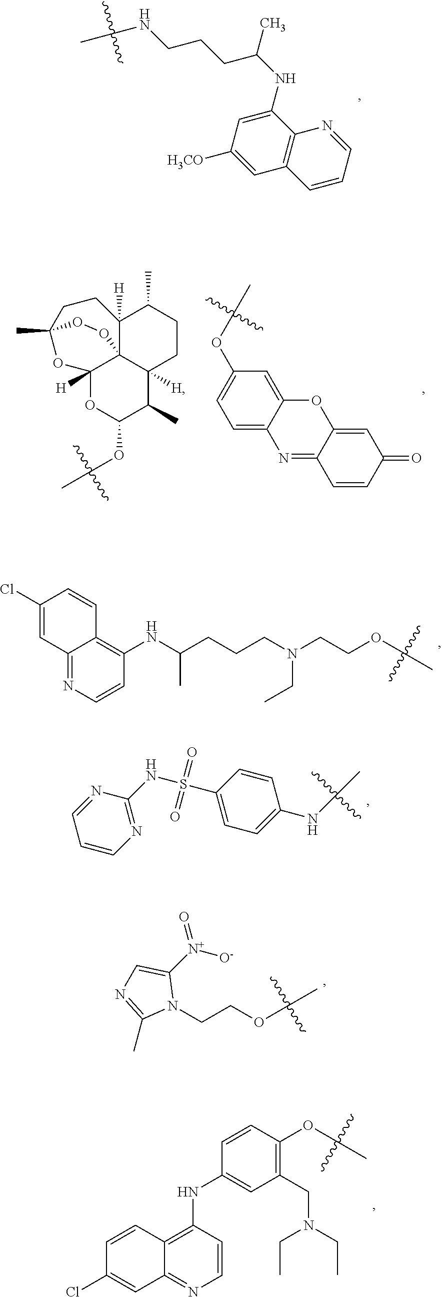

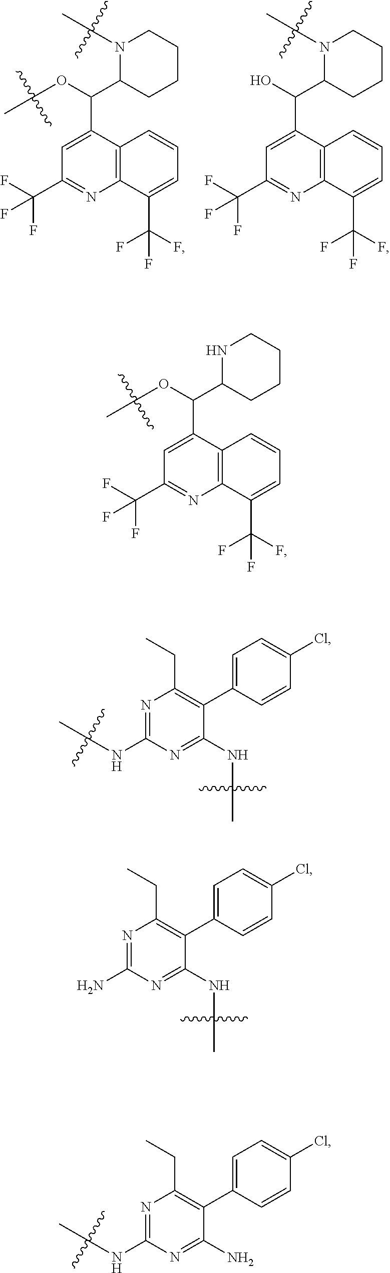

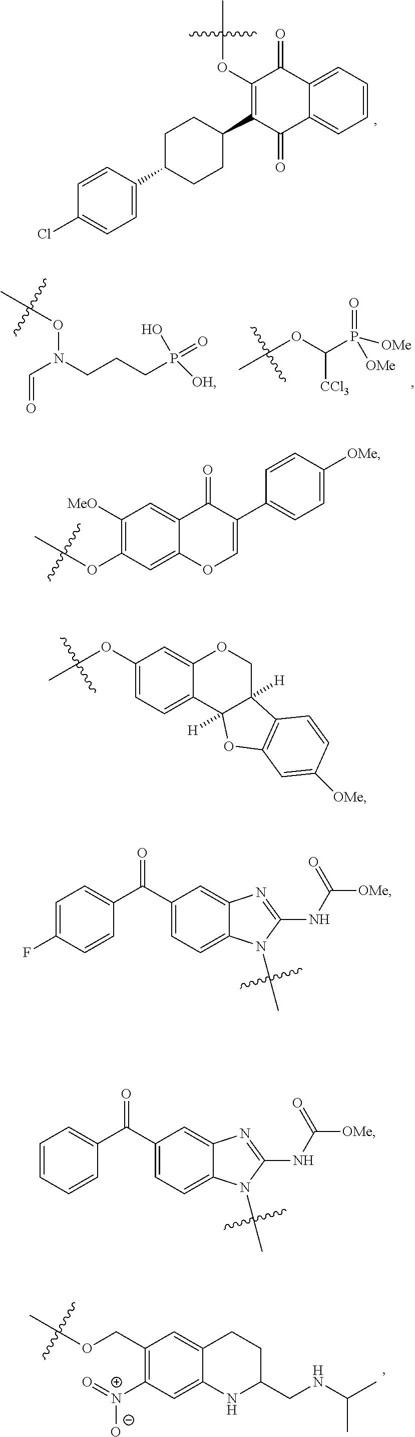

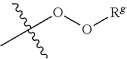

wherein R^{g} is H or substituted or unsubstituted —C₁₋₆ alkyl,

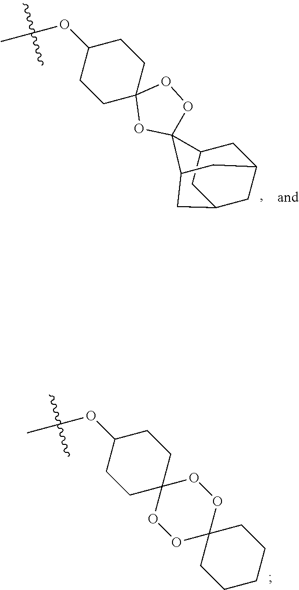 [SMILES: *OC1CCC2(CC1)OOC1(O2)C2CC3CC(C2)CC1C3 ; *OC1CCC2(CC1)OOC1(CCCCC1)OO2]

and
wherein each of L¹, L² and L³, when present, is substituted or unsubstituted and is independently selected from:

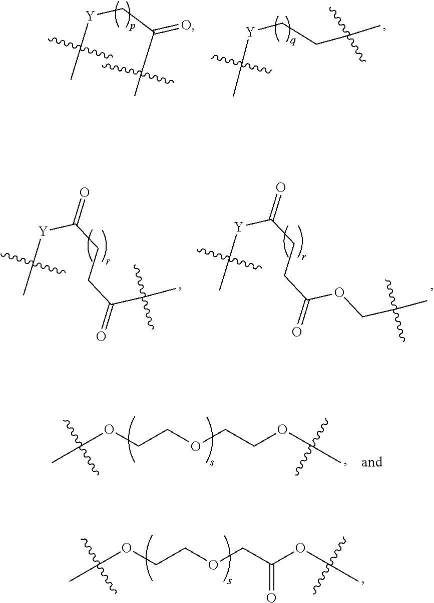

wherein Y is —NR^{f}—, —S—, —O— or —CR^{f1}R^{f2}, wherein each of R^{f}, R^{f1} and R^{f2} is H or substituted or unsubstituted —C₁₋₆ alkyl; each of p, q, r and s is independently 0, 1, 2, 3 or 4; wherein, when L¹, L² or L³ is substituted, L¹, L² or L³ is substituted with one or more groups selected from Substituent Group A.

In some embodiments, the compound of Formula (I″) is a compound of Formula (I″a):

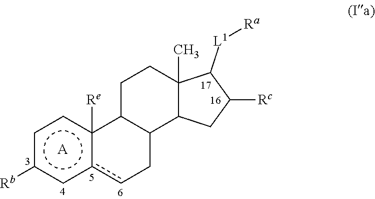

wherein:
R^{a} is selected from:

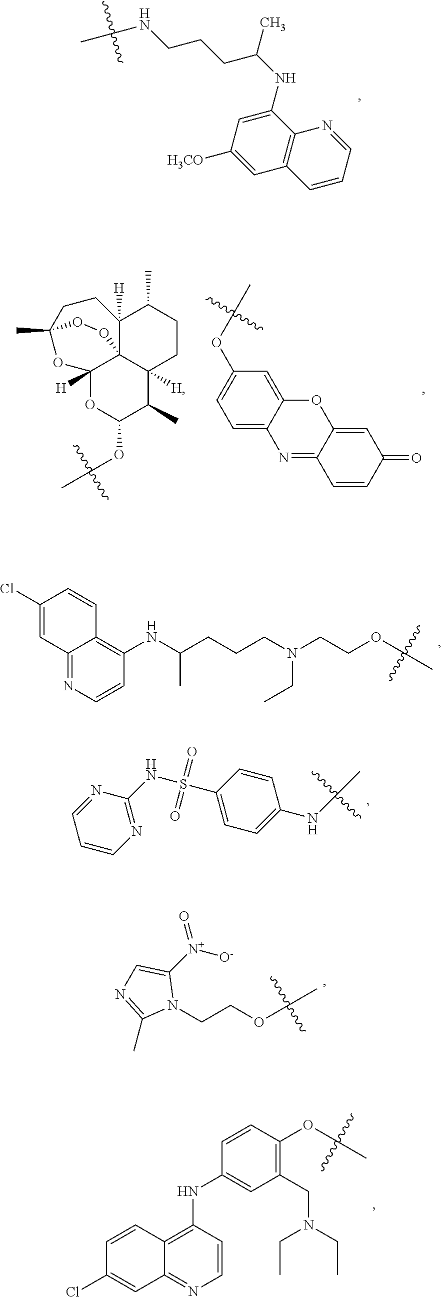

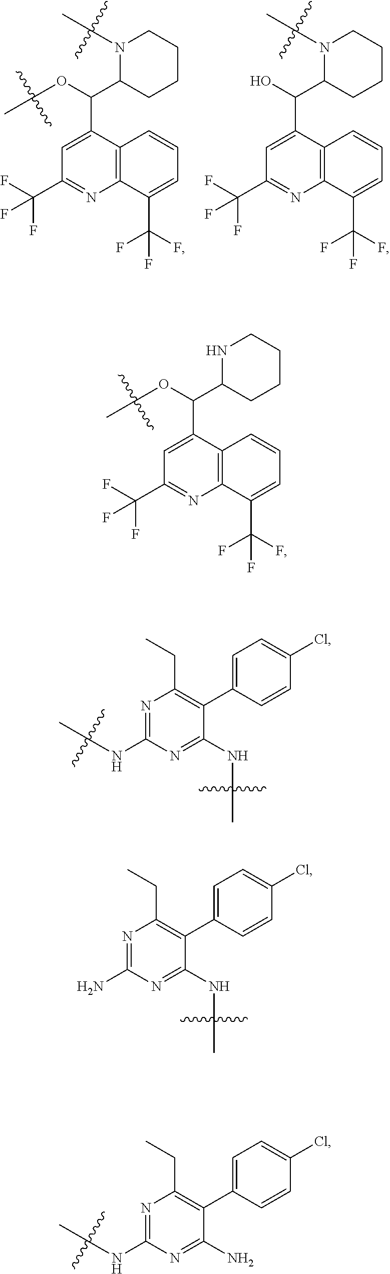

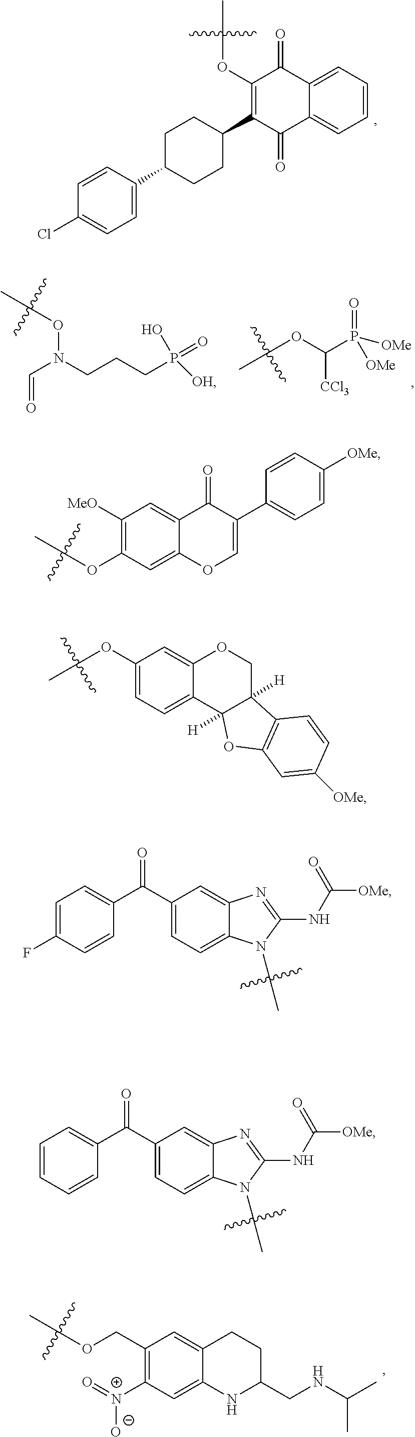

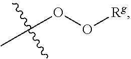

wherein R^{g} is H or substituted or unsubstituted —C₁₋₆ alkyl,

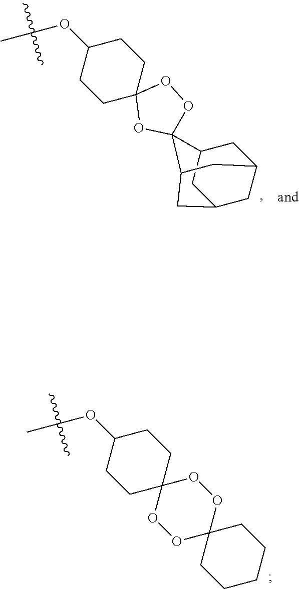 [SMILES: *OC1CCC2(CC1)OOC1(O2)C2CC3CC(C2)CC1C3 ; *OC1CCC2(CC1)OOC1(CCCCC1)OO2]

each of R^{b} and R^{c} is selected from —H, —OH, ═O, substituted or unsubstituted —C₁₋₁₀ alkyl, substituted or unsubstituted —C₂₋₁₀ alkenyl, substituted or unsubstituted —C₂₋₁₀ alkynyl, substituted or unsubstituted —C₃₋₁₀ cycloalkyl; substituted or unsubstituted —C₅₋₁₄ cycloalkenyl, or substituted or unsubstituted —C₈₋₁₄ cycloalkynyl, wherein, when R^{b} or R^{c} is substituted, R^{b} or R^{c} is substituted with one or more groups selected from Substituent Group A;
R^{e} is H or CH₃, or R^{e} is absent;
L¹ may be substituted or unsubstituted and is independently selected from:

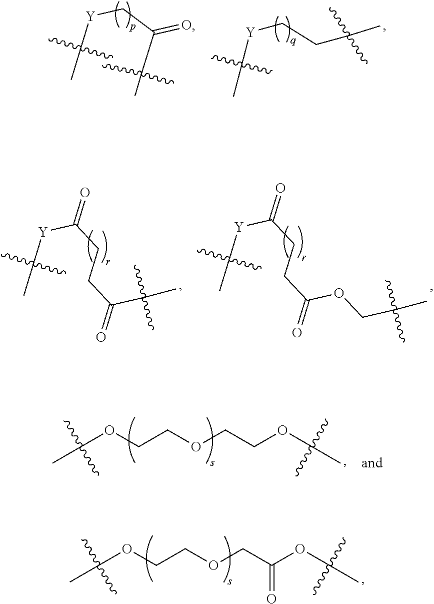

wherein Y is —NR^{f}—, —S—, —O— or —CR^{f1}R^{f2}—, wherein each of R^{f}, R^{f1} and R^{f2} is H or substituted or unsubstituted —C₁₋₆ alkyl; each of p, q, r and s is independently 0, 1, 2, 3 or 4; wherein, when L¹ is substituted, L¹ is substituted with one or more groups selected from Substituent Group A;
ring A may be a saturated or unsaturated ring, or ring A may be an aromatic ring, provided that when ring A is an aromatic ring, the C5-C6 bond is a single bond, R^{e} is absent, and R^{b} is not ═O;
[symbol] is a single bond or a double bond; provided that when the C4-C5 or C5-C10 bond is a double bond, the C5-C6 bond is a single bond; when the C5-C6 bond is a double bond, the C4-C5 and C5-C10 bonds are single bonds; and when the C3 forms a double bond with C2 or C4, R^{b} is not ═O;
or a stereoisomer thereof, or a pharmaceutically acceptable salt thereof.

In some embodiments, the compound of Formula (I″) is a compound of Formula (I′c):

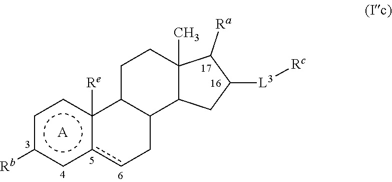

wherein:
each of R^{a} and R^{b} is selected from —H, —OH, ═O, substituted or unsubstituted —C₁₋₁₀ alkyl, substituted or unsubstituted —C₂₋₁₀ alkenyl, substituted or unsubstituted —C₂₋₁₀ alkynyl, substituted or unsubstituted —C₃₋₁₀ cycloalkyl; substituted or unsubstituted —C₅₋₁₄ cycloalkenyl, or substituted or unsubstituted —C₈₋₁₄ cycloalkynyl, wherein, when R^{a} or R^{b} is substituted. R^{a} or R^{b} is substituted with one or more groups selected from Substituent Group A;
R^{c} is selected from:

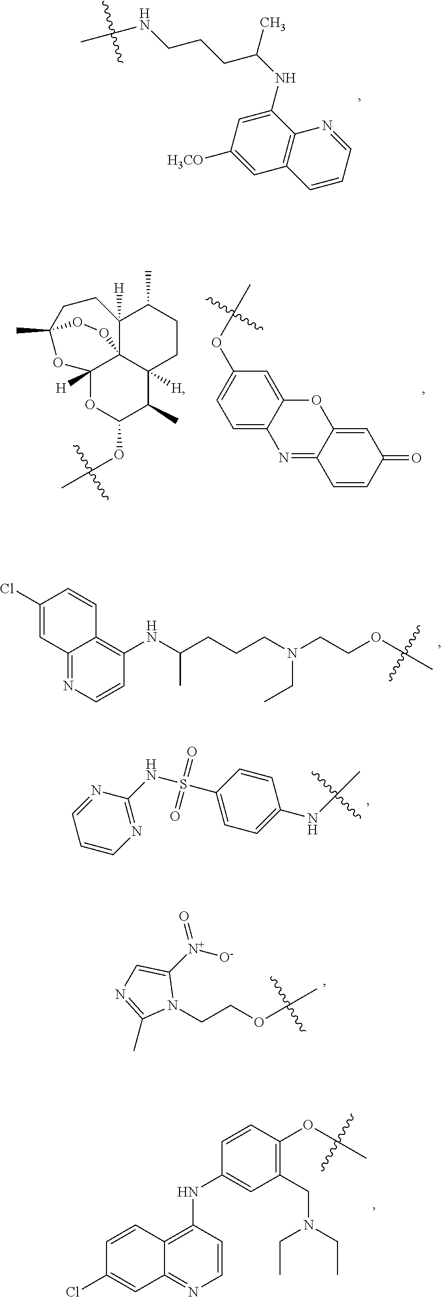

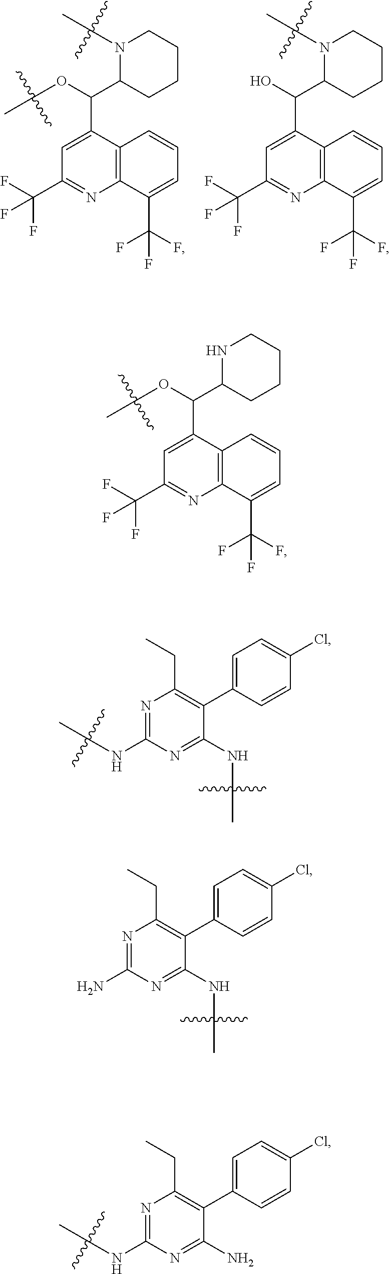

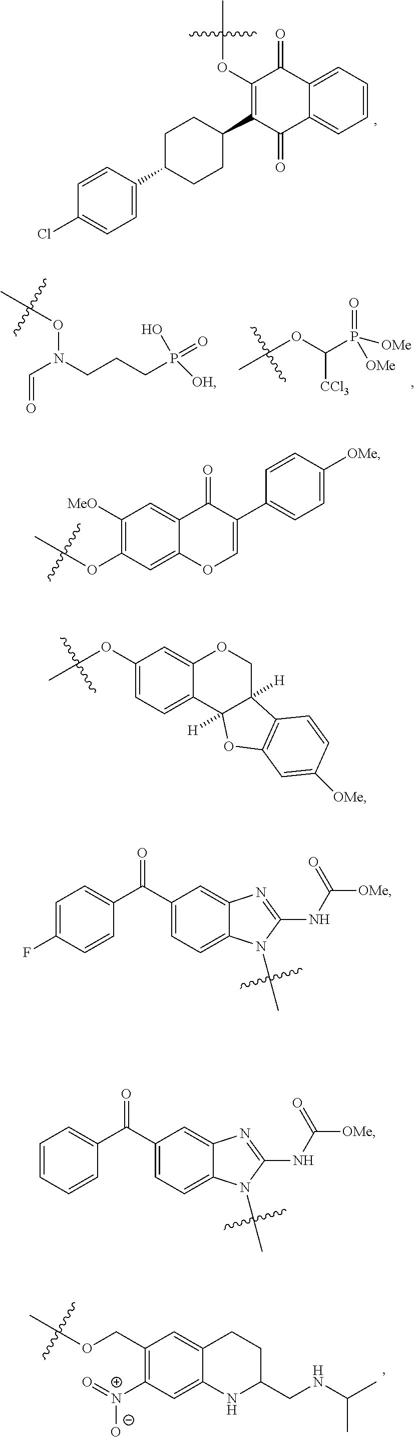

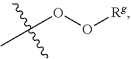

wherein R^{g} is H or substituted or unsubstituted —C₁₋₆ alkyl,

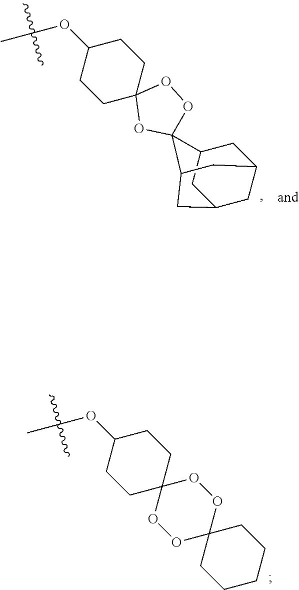 [SMILES: *OC1CCC2(CC1)OOC1(O2)C2CC3CC(C2)CC1C3 ; *OC1CCC2(CC1)OOC1(CCCCC1)OO2]

R^{e} is H or CH₃, or R^{e} is absent;
L³ may be substituted or unsubstituted and is independently selected from:

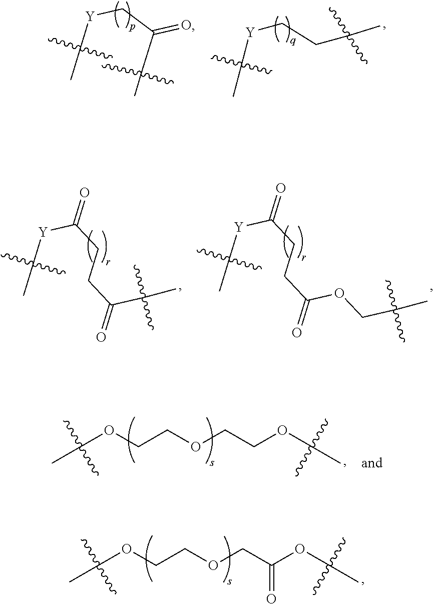

wherein Y is —NR^{f}—, —S—, —O— or —CR^{f1}R^{f2}—, wherein each of R^{f}, R^{f1} and R^{f2} is H or substituted or unsubstituted —C₁₋₆ alkyl; each of p, q, r and s is independently 0, 1, 2, 3 or 4; wherein, when L³ is substituted, L³ is substituted with one or more groups selected from Substituent Group A;
ring A may be a saturated or unsaturated ring, or ring A may be an aromatic ring, provided that when ring A is an aromatic ring, the C5-C6 bond is a single bond, R^{a} is absent, and R^{b} is not ═O;
[symbol] is a single bond or a double bond; provided that when the C4-C5 or C5-C10 bond is a double bond, the C5-C6 bond is a single bond; when the C5-C6 bond is a double bond, the C4-C5 and C5-C10 bonds are single bonds; and when the C3 forms a double bond with C2 or C4, R^{b} is not ═O;
or a stereoisomer thereof, or a pharmaceutically acceptable salt thereof.

In some embodiments, R^{a} of Formula (I″a) or R^{c} of Formula (I″c) is selected from any of:

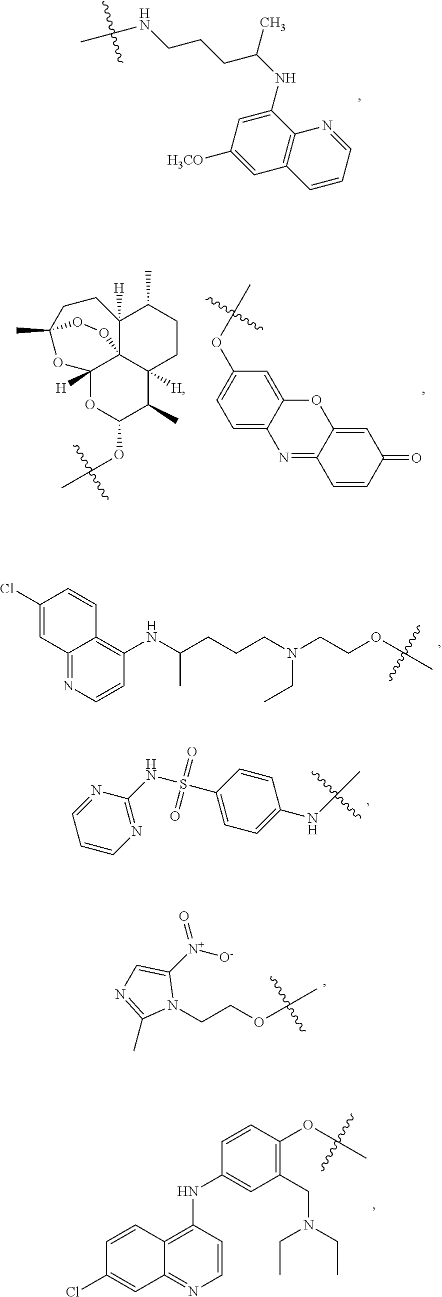

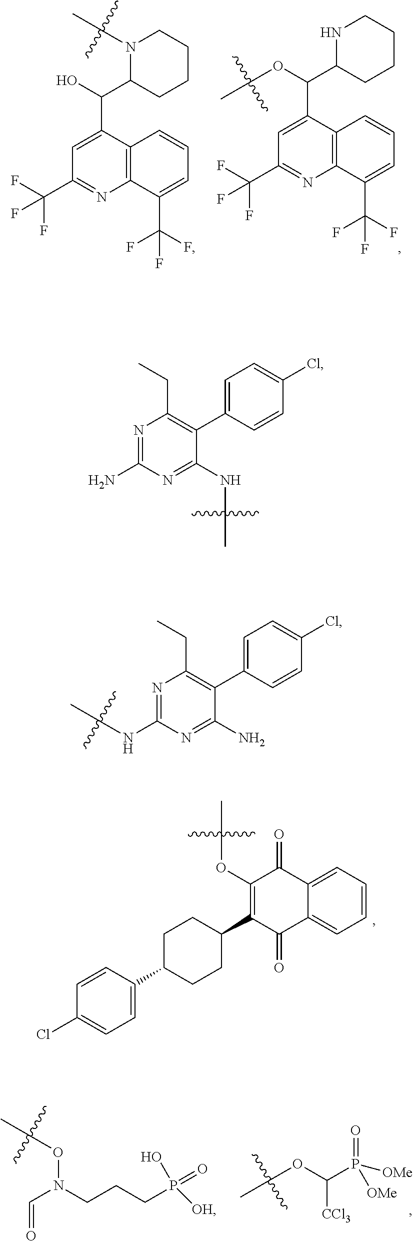

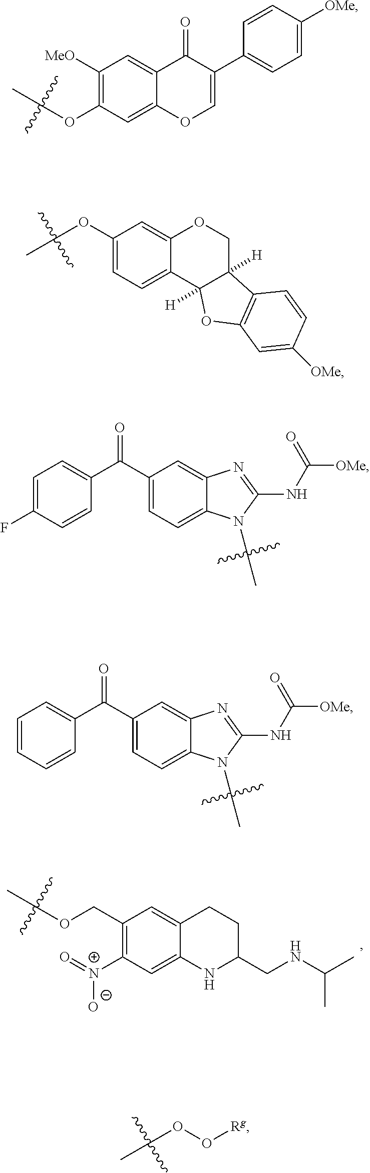

wherein R^{g} is H or substituted or unsubstituted —C₁₋₆ alkyl,

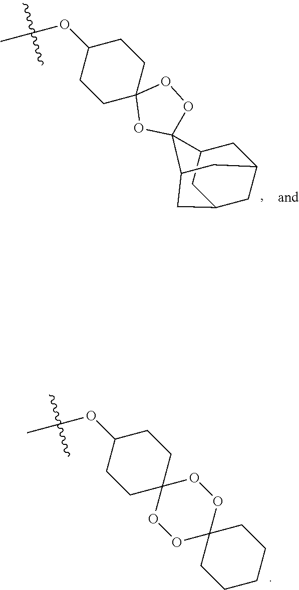 [SMILES: *OC1CCC2(CC1)OOC1(O2)C2CC3CC(C2)CC1C3 ; *OC1CCC2(CC1)OOC1(CCCCC1)OO2]

In some embodiments, L¹ of Formula (I″a) or L³ of Formula (I″c) is selected from any of:

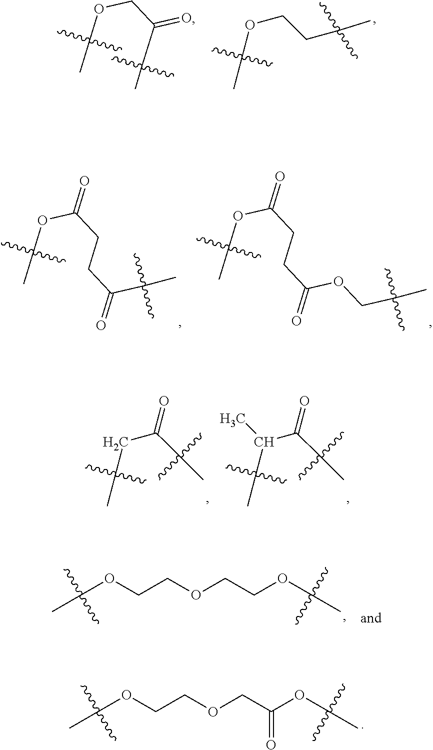

In some embodiments, the compound of Formula (I″) is not:

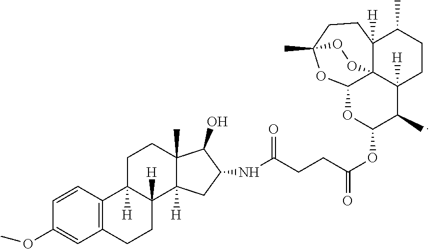

In some embodiments, the compound of Formula (I″) is not:

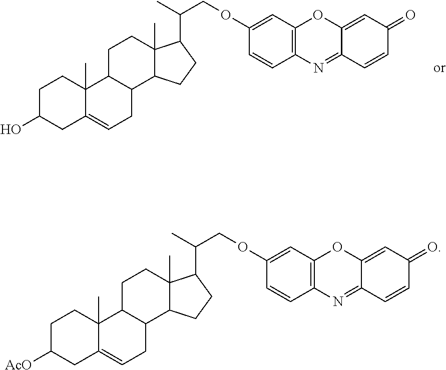 [SMILES: CC(COc1ccc2nc3ccc(=O)cc-3oc2c1)C1CCC2C3CC=C4CC(O)CCC4(C)C3CCC12C ; CC(=O)OC1CCC2(C)C(=CCC3C2CCC2(C)C(C(C)COc4ccc5nc6ccc(=O)cc-6oc5c4)CCC32)C1]

In some embodiments, the compound of Formula (I″) is not:

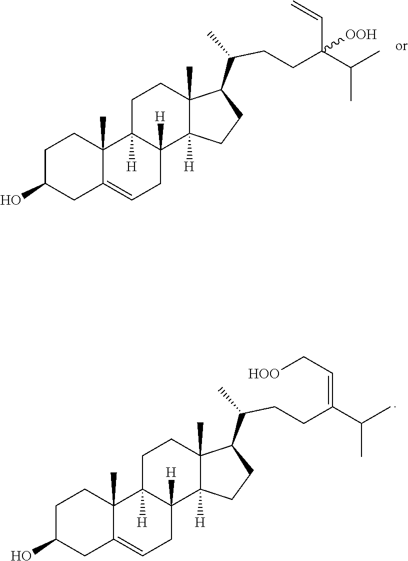

In some embodiments, the compound of Formula (I″) does not have the following group substituted at the C3 position of the steroid:

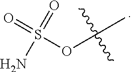 [SMILES: *OS(N)(=O)=O]

In some embodiments, the compound of Formula (I″) is not any one of the following:

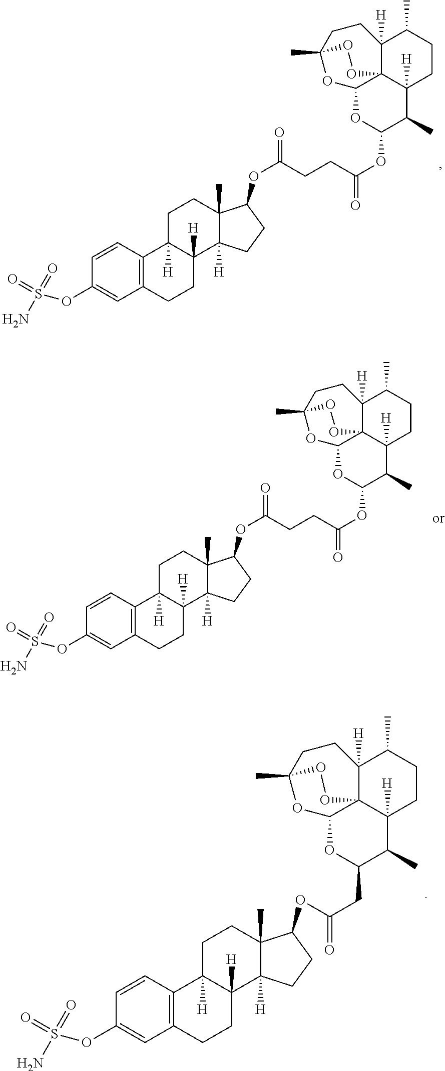

Thus, in some embodiments, the present invention provides a compound of Formula (I″):

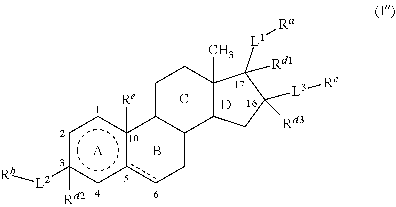

wherein:
each of R^{a} and R^{c} is independently selected from —H, —OH, ═O, substituted or unsubstituted —C₁₋₁₀ alkyl, substituted or unsubstituted —C₂₋₁₀ alkenyl, substituted or unsubstituted —C₂₋₁₀ alkynyl, substituted or unsubstituted —C₃₋₁₀ cycloalkyl, substituted or unsubstituted —C₅₋₁₄ cycloalkenyl, substituted or unsubstituted —C₈₋₁₄ cycloalkynyl, and an anti-parasite moiety; wherein at least one of R^{a} and R^{c} is an anti-parasite moiety;
R^{b} is selected from —H, —OH, ═O, substituted or unsubstituted —C₁₋₁₀ alkyl, substituted or unsubstituted —C₂₋₁₀ alkenyl, substituted or unsubstituted —C₂₋₁₀ alkynyl, substituted or unsubstituted —C₃₋₁₀ cycloalkyl, substituted or unsubstituted —C₅₋₁₄ cycloalkenyl, and substituted or unsubstituted —C₈₋₁₄ cycloalkynyl;
provided that: when R^{a} is —OH, L¹ is absent; when R^{a} is ═O, L¹ and R^{d1} are absent; when R^{b} is —OH, L² is absent; when R^{b} is ═O, L² is absent, R² is absent, and the C2-C3 bond and the C3-C4 bond are single bonds; when R^{c} is —OH, L³ is absent; when R^{c} is ═O, L³ and R^{d3} are absent;
when R^{a}, R^{b} or R^{c} is substituted, R^{a}, R^{b} or R^{c} is substituted with one or more groups selected from Substituent Group A;
R^{d1}, when present, is H or is a group that, together with R^{a}, forms a substituted or unsubstituted ozonide ring or a substituted or unsubstituted tetraoxane ring, wherein the optional substituent may be a —C₃₋₁₀ cycloalkyl; provided that when R^{d1} and R^{a} form a substituted or unsubstituted ozonide ring or a substituted or unsubstituted tetraoxane ring, L¹ is absent; wherein, when the ozonide or tetraoxane ring is substituted, the ozonide or tetraoxane ring is substituted with one or more —C₃₋₁₀ cycloalkyl groups;
R^{d2}, when present, is H; provided that when R^{d2} is present, the C2-C3 bond and the C3-C4 bond are single bonds;
R^{d3}, when present, is H or is a group that, together with R^{c}, forms a substituted or unsubstituted ozonide ring or a substituted or unsubstituted tetraoxane ring, wherein the optional substituent may be a —C₃₋₁₀ cycloalkyl; provided that when R^{d3} and R^{c} form a substituted or unsubstituted ozonide ring or a substituted or unsubstituted tetraoxane ring, L³ is absent; wherein, when the ozonide or tetraoxane ring is substituted, the ozonide or tetraoxane ring is substituted with one or more —C₃₋₁₀ cycloalkyl groups;
R^{e} is H or CH₃, or R^{e} is absent;
when present, L¹ is a group that provides a covalent linkage between R^{a} and the C17 of ring D;
when present, L² is a group that provides a covalent linkage between R^{b} and the C3 of ring A;
when present, L³ is a group that provides a covalent linkage between R^{c} and the C16 of ring D;
ring A may be a saturated or unsaturated ring, or ring A may be an aromatic ring, provided that when ring A is an aromatic ring, the C5-C6 bond is a single bond. R² is absent. R^{e} is absent, and R^{b} is not ═O;
[symbol] is a single bond or a double bond; provided that when the C4-C5 or C5-C10 bond is a double bond, the C5-C6 bond is a single bond; when the C5-C6 bond is a double bond, the C4-C5 and C5-C10 bonds are single bonds; and when the C3 forms a double bond with C2 or C4, R^{b} is not ═O;
or a stereoisomer thereof, or a salt thereof;
wherein the anti-parasite moiety is selected from:

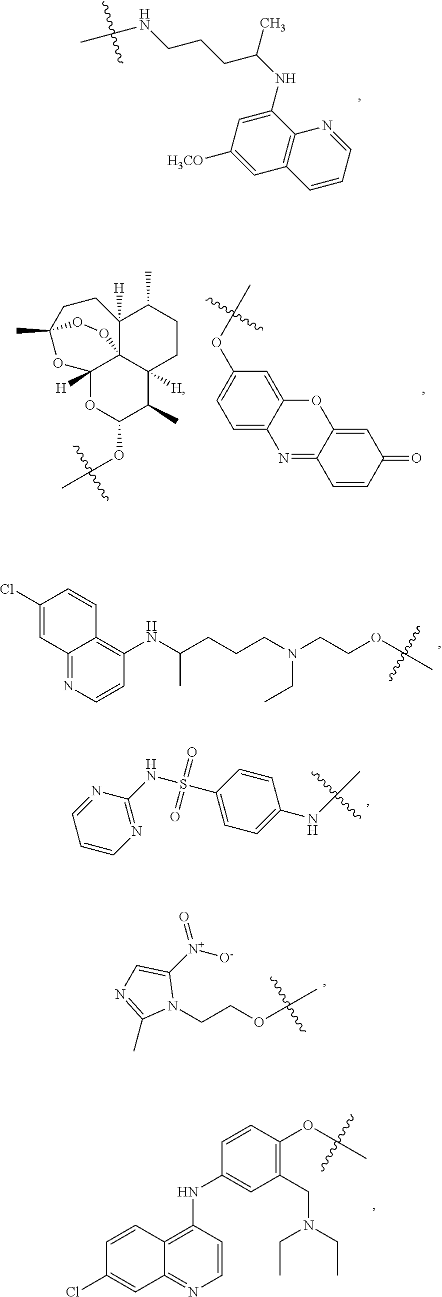

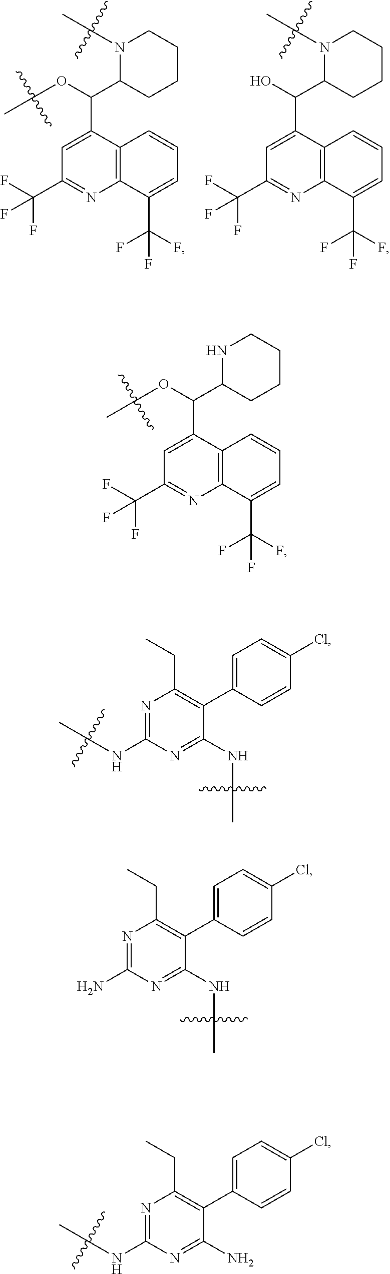

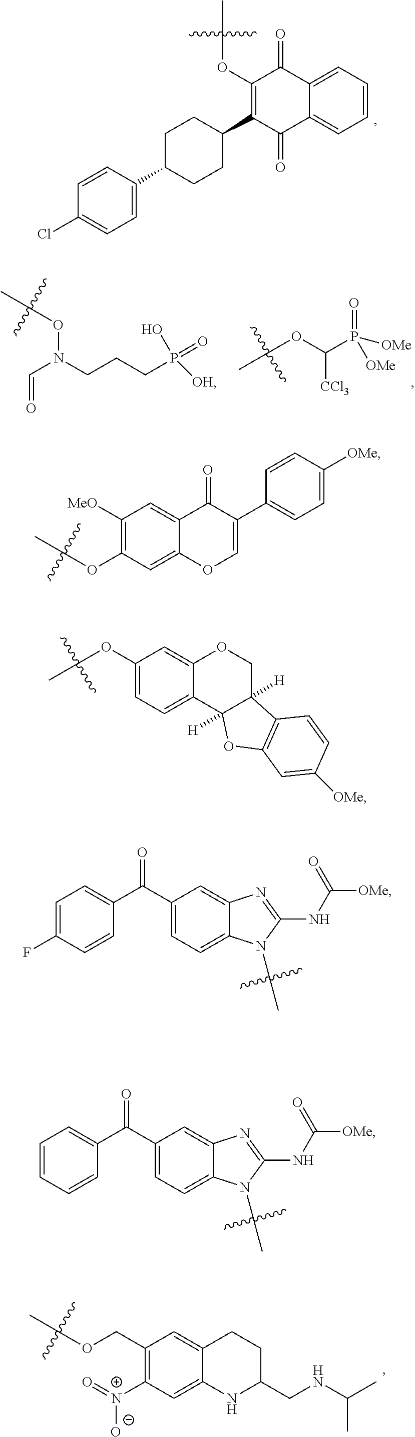

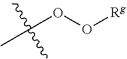

wherein R^{g} is H or substituted or unsubstituted —C₁₋₆ alkyl,

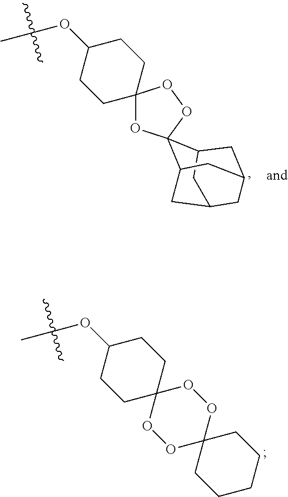 [SMILES: *OC1CCC2(CC1)OOC1(O2)C2CC3CC(C2)CC1C3 ; *OC1CCC2(CC1)OOC1(CCCCC1)OO2]

and
wherein each of L¹, L² and L³, when present, is substituted or unsubstituted and is independently selected from:

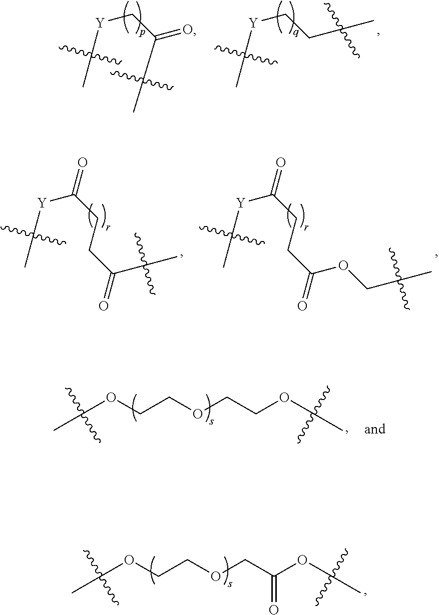

wherein Y is —NR^{f}—, —S—, —O— or —CR^{f1}R^{f2}—, wherein each of R^{a}, R^{f1} and R^{f2} is H or substituted or unsubstituted —C₁₋₆ alkyl; each of p, q, r and s is independently 0, 1, 2, 3 or 4; wherein, when L¹, L² or L³ is substituted, L¹, L² or L³ is substituted with one or more groups selected from Substituent Group A;
wherein the compound of Formula (I″) is not:

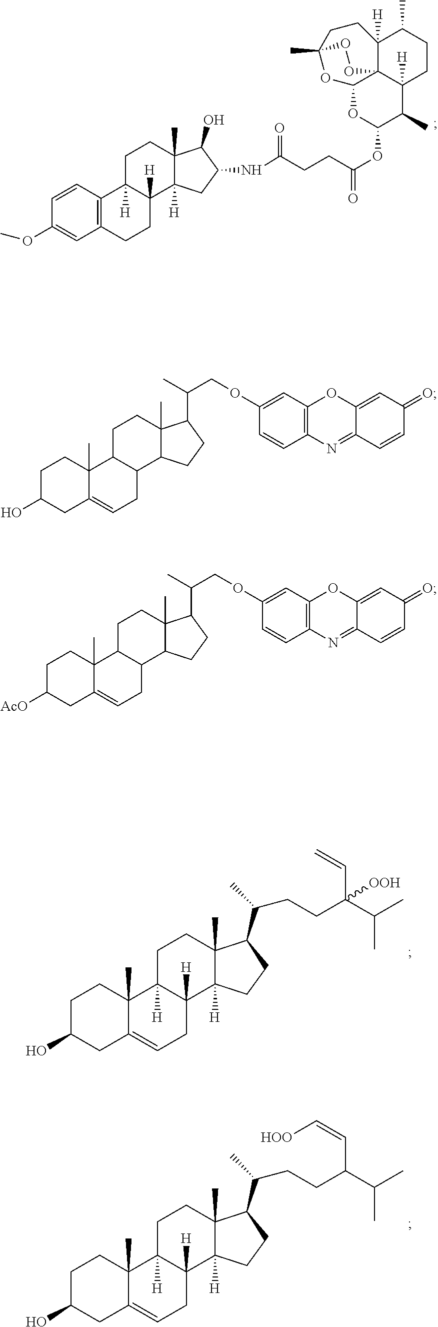

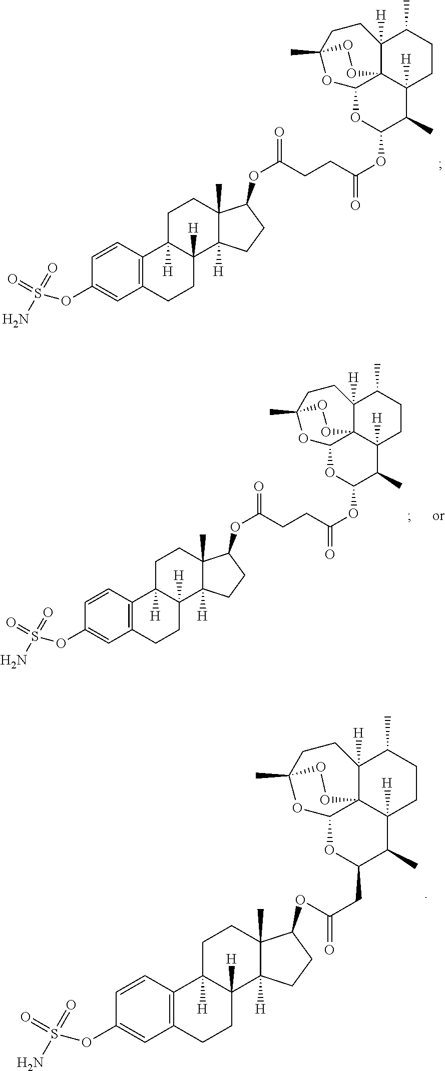

Unless otherwise defined, the term “optionally substituted”, or “substituted or unsubstituted”, as used herein indicated a group may or may not be substituted with 1, 2, 3, 4 or more groups, preferably 1, 2 or 3 groups, more preferably 1 or 2 groups, independently selected from Substituent Group A, which consists of alkyl (e.g. C₁₋₆alkyl), alkenyl (e.g. C₂₋₆alkenyl), alkynyl (e.g. C₂₋₆alkynyl), cycloalkyl (e.g. C₃₋₈cycloalkyl), hydroxyl, oxo, heteroalkyl, alkoxy (e.g. C₁₋₆alkoxy), aryloxy, arylC₁₋₆alkoxy, halo, haloC₁₋₆alkyl (such as —CF₃ and —CHF₂), haloC₁₋₆alkoxy (such as —OCF₃ and —OCHF₂), carboxyl, esters, cyano, nitro, amino, substituted amino, disubstituted amino, acyl, substituted acyl, ketones, amides, aminoacyl, substituted amides, disubstituted amides, thiol, alkylthio, thioxo, sulfates, sulfonates, sulfinyl, substituted sulfinyl, sulfonyl, substituted sulfonyl, sulfonylamides, substituted sulfonamides, disubstituted sulfonamides, aryl, arylC₁₋₆alkyl, heterocyclylC₁₋₆alkyl, arylC₂₋₆alkenyl, heterocyclylC₂₋₆alkenyl, arylC₂₋₆alkynyl, heterocyclylC₂₋₆alkynyl, heteroarylC₁₋₆alkyl, heteroarylC₂₋₆alkenyl, heteroarylC₂₋₆alkynyl, heterocyclyl and heteroaryl, wherein each alkyl, alkenyl, alkynyl, cycloalkyl, aryl and heterocyclyl and groups containing them may be further optionally substituted. Optional substituents in the case of heterocycles containing N may also include but are not limited to C₁₋₆alkyl i.e. N—C₁₋₆alkyl.

For optionally substituted “alkyl”, “alkenyl” and “alkynyl”, the optional substituent or substituents are preferably selected from amino, substituted amino, disubstituted amino, aryl, halo (e.g. F, Cl, Br, I), heterocyclyl, C₃₋₈cycloalkyl, C₁₋₆alkoxy, hydroxyl, oxo, aryloxy, haloC₁₋₆ alkyl, haloC₁₋₆alkoxyl and carboxyl. Each of these optional substituents may also be optionally substituted with any of the optional substituents referred to above, where nitro, amino, substituted amino, cyano, heterocyclyl (including non-aromatic heterocyclyl and heteroaryl), C₁₋₆ alkyl, C₂₋₆alkenyl, C₂₋₆alkynyl, C₁₋₆alkoxyl, haloC₁₋₆alkyl, haloC₁₋₆alkoxy, halo, hydroxyl and carboxyl are preferred.

For substituted or unsubstituted “ammonium”, the optional substituent or substituents are preferably selected from C₁₋₃alkyl, such as C₁alkyl, C₂alkyl or C₃alkyl. More preferably, the optional substituent or substituents are CH₃.

For substituted or unsubstituted “amino”, the optional substituent or substituents are preferably selected from C₁₋₃alkyl, such as C₁alkyl, C₂alkyl or C₃alkyl. More preferably, the optional substituent or substituents are CH₃.

It will be understood that suitable derivatives of aromatic heterocyclyls containing nitrogen include N-oxides thereof.

The salts of the compounds of the Formula (I) are pharmaceutically acceptable. When compounds of the Formula (I) contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, lithium, calcium, ammonium, organic ammonium (e.g. alkylammonium), or magnesium salt, or a similar salt. When compounds contain relatively basic functionalities, acid addition salts can be obtained by contacting the neutral form of such compounds with a sufficient amount of the desired acid, either neat or in a suitable inert solvent.

Examples of pharmaceutically acceptable acid addition salts include those derived from inorganic acids like hydrochloric, hydrobromic, boric, nitric, carbonic, monohydrogencarbonic, phosphoric, monohydrogenphosphoric, dihydrogenphosphoric, sulfuric, sulfamic, monohydrogensulfuric, hydroiodic, or phosphorous acids and the like, as well as the salts derived from relatively nontoxic organic acids like acetic, propionic, isobutyric, butyric, maleic, hydroxymaleic, malonic, benzoic, succinic, oxalic, phenylacetic, trihaloacetic (e.g. trifluoroacetic), suberic, fumaric, lactic, mucic, gluconic, mandelic, phthalic, benzenesulfonic, p-tolylsulfonic, salicylic, sulfanilic, aspartic, glutamic, citric, tartaric, methanesulfonic, trihalomethanesulfonic, edetic, stearic, palmitic, oleic, lauric, pantothenic, tannic, ascorbic, valeric acids and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the Formula (I) contain both basic and acidic functionalities that allow the compounds to be converted into either base or acid addition salts. Other pharmaceutically acceptable carriers known to those of skill in the art are suitable for the present invention.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

### Preparation Methods

The compounds of Formula (I) may be synthesised by methods known in the art. Various synthetic schemes are described below and in the Examples. The Examples describe the preparation of various specific compounds of Formula (I). A person skilled in the art would be able to modify the synthetic schemes described below and in the Examples to prepare other compounds of Formula (I) or salts thereof.

### Applications

The anti-parasitic activity of the compounds of Formula (I) makes these compounds useful in clinical applications to treat or prevent various parasitic infections, such as protozoan parasite infections, including various apicomplexan parasite infections.

Thus, the present invention also provides the following:
a composition comprising a compound of Formula (I) or a salt thereof, and a suitable carrier, adjuvant or diluent;
a pharmaceutical composition comprising a compound of Formula (I) or a pharmaceutically acceptable salt thereof, and a pharmaceutically acceptable carrier, adjuvant or diluent;
a method of treating or preventing a parasitic infection, such as a protozoan parasite infection, in a subject, the method comprising administering to the subject an effective amount of a compound of Formula (I) or a pharmaceutically acceptable salt thereof;
a method of inhibiting the proliferation of a parasite, such as a protozoan parasite, the method comprising contacting the parasite with an effective amount of a compound of Formula (I) or a salt thereof;
use of a compound of Formula (I) or a pharmaceutically acceptable salt thereof, in the manufacture of a medicament for the treatment or prevention of a parasitic infection, such as a protozoan parasite infection, in a subject;
a method of treating or preventing a parasitic infection, such as an apicomplexan parasite infection, in a subject, the method comprising administering to the subject an effective amount of a compound of Formula (I) or a pharmaceutically acceptable salt thereof;
a method of inhibiting the proliferation of a parasite, such as an apicomplexan parasite, the method comprising contacting the parasite with an effective amount of a compound of Formula (I) or a salt thereof;
use of a compound of Formula (I) or a pharmaceutically acceptable salt thereof, in the manufacture of a medicament for the treatment or prevention of a parasitic infection, such as an apicomplexan parasite infection, in a subject;
a composition, a pharmaceutical composition, a method, or a use as described above in any of the preceding points where Formula (I) is any of the following: Formula (Ia), Formula (Ib), Formula (Ic), Formula (I″), Formula (I″a), or Formula (I″c), as described herein.

In one form, the compound of Formula (I) or a pharmaceutically acceptable salt thereof can be used to treat infections by parasites.

One aspect therefore provides a method of treating or preventing an infection by a parasite, comprising administering an effective amount of a compound of Formula (I), or a pharmaceutically acceptable salt thereof.

In one embodiment, the parasite is a helminth. Examples of helminths include nematodes, trematodes and cestodes. Examples of nematodes include Ascaris spp., Enterobios spp., Filarioidea spp., Onchocerca spp., Rhabditis spp., Trichuris spp., and Ancylostoma spp. Examples of trematodes include flukes, such as Schistosoma spp., Fasciola spp. and Dicrocoelium spp. Examples of cestodes include tapeworms, such as Taenia spp., Hymenolepis spp. and Echinococcus spp.

In one embodiment, the parasite is a protozoan parasite. In one embodiment the protozoan parasite is selected from flagellates (eg., Giardia, Trichomonas, Leishmania and trypanosomes), ciliates (e.g., Balantidium coli), amoeba (e.g., Entamoeba histolytica), and apicomplexans (e.g., Toxoplasma, Plasmodium, Eimeria, Theileria, Babesia, Sarcocystis, and Cryptosporidium). Typically, the parasite is a protozoan parasite that is reliant on host cholesterol for growth and/or survival.

In one embodiment, the flagellate is a kinetoplastid (e.g. Leishmania such as Leishmania tarentolae, and trypanosomes).

In one embodiment, the parasite is an apicomplexan parasite.

One embodiment therefore provides a method of treating or preventing an infection by an apicomplexan parasite, comprising administering an effective amount of a compound of Formula (I), or a pharmaceutically acceptable salt thereof. Examples of apicomplexan parasites include Toxoplasma spp., Plasmodium spp., Eimeria spp., Theileria spp., Babesia spp., Sarcocystis spp., and Cryptosporidium spp.

In one embodiment, the compound of Formula (I) can be used to treat an infection by the apicomplexan parasite Plasmodium spp. Accordingly, the compound of formula (I) can be used to treat or prevent malaria.

One embodiment therefore provides a method for treating or preventing malaria in a subject, comprising administering an effective amount of a compound of Formula (I), or a pharmaceutically acceptable salt thereof.

One embodiment provides a method for treating or preventing a Plasmodium spp. infection in a subject, comprising administering an effective amount of a compound of Formula (I), or a pharmaceutically acceptable salt thereof.

Examples of Plasmodium spp. include Plasmodium falciparum, Plasmodium vivax, Plasmodium ovale curtisi, Plasmodium ovale wallikeri, Plasmodium malariae and Plasmodium knowlesi. In one embodiment, the Plasmodium spp. is P. vivax or P. falciparum. In one embodiment, the Plasmodium spp. is P. falciparum. In one embodiment, the Plasmodium spp. is P. vivax.

In one form, the compound of Formula (I) or a pharmaceutically acceptable salt thereof can be used to treat infections by the apicomplexan parasite Toxoplasma gondii.

One embodiment provides a method for treating or preventing toxoplasmosis in a subject, comprising administering an effective amount of a compound of Formula (I) or a pharmaceutically acceptable salt thereof.

One aspect provides a method for treating or preventing a Toxoplasma gondii infection in a subject, comprising administering an effective amount of a compound of Formula (I) or a pharmaceutically acceptable salt thereof.

In one form, the compound of Formula (I) or a pharmaceutically acceptable salt thereof can be used to treat infections by apicomplexan parasites of the family Eimeriidae. Parasites of the family Eimeriidae include Eimeria spp. and Isospora spp. Emeria spp. and Isospora spp. are the causative agents of coccidiosis. Accordingly, the compound of formula (I) can be used to treat or prevent coccidiosis.

One embodiment provides a method of treating or preventing an Eimeria spp. or Isospora spp. infection in a subject, comprising administering an effective amount of a compound of Formula (I), or a pharmaceutically acceptable salt thereof. Examples of Eimeria species include Eimeria acervulina, Eimeria adenoeides, Eimeria brunetti, Eimeria colchici, Eimeria curvata, Eimeria dispersa, Eimeria duodenalis, Eimeria fraterculae, Eimeria gallopavonis, Eimeria innocua, Eimeria praecox, Eimeria maxima, Eimeria meleagridis, Eimeria meleagrimitis, Elmeria mitis, Eimeria necatrix, Eimeria phasiani, Eimeria procera, Eimeria purpureicephali, Emeria tenella, Eimeria ahsata, Eimeria alabamensis, Eimeria alijevi, Eimeria aspheronica, Eimeria arloingi, Eimeria arundeli, Eimeria bakuensis, Eimeria bovis, Eimeria cameli, Eimeria caprina, Emeria caprovina, Eimeria christenseni, Eimeria clethrionomyis, Eimeria coecicola, Eimeria contorta, Eimera couesii, Emeria crandallis, Eimeria dammahensis, Eimeria dowleri, Elmeria exigua, Eimeria falciformis, Eimeria farasanii, Eimeria fernsi, Eimeria navescens, Eimeria gallatii, Emeria granulosa, Eimeria hird, Eimeria intestinalis, Emeria irresidua, Elmeria intricate, Eimeriajolchijevi, Eimeria krijgsmanni, Eimeria larimerensis, Eimeria macusaniensis, Eimeria magna, Eimeria marconii, Eimeria media, Eimeria melanuric, Eimeria myoxi, Eimeria nagpurensis, Eimeria nieschulzi, Elmeria ninakohlyakimovae, Eimeria ovinoidalis, Eimeria pallida, Eimeria palustis, Eimeria papillate, Eimeria perforans, Eimeria phocae, Eimeria pileate, Eimeria pipistrellus, Eimeria piriformis, Eimeria prionotemni, Eimeria procyonis, Eimeria punctate, Eimeria roobroucki, Eimeria saudiensis, Eimeria sealanderi, Eimeda separate, Eimeria sthedai, Eimeria ursini, Eimeria vermiformis, Emeria weybridgensis, Eimeria wobati, Eimeria zuernii.

Examples of species of Isospora species include I. hominis and I. belli, I. bigemina, I. rivolta, and I. felis.

One embodiment provides a method of treating or preventing Coccidiosis in a subject, comprising administering an effective amount of a compound of Formula (I), or a pharmaceutically acceptable salt thereof.

In one embodiment, the parasite is a flagellate, such as Leishmania (e.g. Leishmania tarentolae).

One embodiment provides a method of treating or preventing Leishmania parasite infection in a subject, comprising administering an effective amount of a compound of Formula (I), or a pharmaceutically acceptable salt thereof.

### Pharmaceutical Compositions

The invention also provides a pharmaceutical composition comprising a compound of Formula (I) or a pharmaceutically acceptable salt thereof, and a pharmaceutically acceptable carrier.

In some embodiments, the compound of Formula (I) or a pharmaceutically acceptable salt thereof may be used in combination with one or more other agents.

Accordingly, in some embodiments, the pharmaceutical composition may further comprise, or be administered in combination with, one or more other agents. For example, the pharmaceutical composition may further comprise, or be administered in combination with, agents useful in treating parasitic infections, such as apicomplexan parasite infections.

It will be understood that the combined administration of a compound of Formula (I) or a pharmaceutically acceptable salt thereof with the one or more other agents may be concurrent, sequential, or separate administration.

The compound of Formula (I) or a pharmaceutically acceptable salt thereof may also be used in combination with further active agents useful in the treatment of at least one symptom of the disease or condition associated with parasitic infections, such as apicomplexan parasite infections.

The term “composition” encompasses formulations comprising the active ingredient with conventional carriers and excipients, and also formulations with encapsulating materials as a carrier to provide a capsule in which the active ingredient (with or without other carriers) is surrounded by the encapsulation carrier. In pharmaceutical compositions, the carrier is “pharmaceutically acceptable” meaning that it is compatible with the other ingredients of the composition and is not deleterious to a subject. The pharmaceutical compositions of the present invention may contain other agents or further active agents as described above, and may be formulated, for example, by employing conventional solid or liquid vehicles or diluents, as well as pharmaceutical additives of a type appropriate to the mode of desired administration (for example, excipients, binders, preservatives, stabilisers, flavours, etc.) according to techniques such as those well known in the art of pharmaceutical formulation (See, for example, Remington: The Science and Practice of Pharmacy, 21st Ed., 2005, Lippincott Williams & Wilkins).

The pharmaceutical composition may be suitable for oral, rectal, nasal, topical (including dermal, buccal and sub-lingual), or parenteral (including intramuscular, sub-cutaneous and intravenous) administration.

The compound of Formula (I) or a pharmaceutically acceptable salt thereof (sometimes referred to below as the “compound(s) of the invention”), together with a conventional adjuvant, carrier, or diluent, may thus be placed into the form of pharmaceutical compositions and unit dosages thereof. The pharmaceutical composition may be a solid, such as a tablet or filled capsule, or a liquid such as solution, suspension, emulsion, elixir, or capsule filled with the same, for oral administration. The pharmaceutical composition may also be in the form of suppositories for rectal administration or in the form of sterile injectable solutions for parenteral (including subcutaneous) use.

Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

For preparing pharmaceutical compositions from the compounds of the invention, pharmaceutically acceptable carriers can be either solid or liquid. Solid form preparations include powders, tablets, pills, capsules, cachets, suppositories, and dispensable granules. A solid carrier can be one or more substances which may also act as diluents, flavouring agents, solubilisers, lubricants, suspending agents, binders, preservatives, tablet disintegrating agents, or an encapsulating material.

Suitable carriers are magnesium carbonate, magnesium stearate, talc, sugar, lactose, pectin, dextrin, starch, gelatine, tragacanth, methylcellulose, sodium carboxymethylcellulose, a low melting wax, cocoa butter, and the like. Tablets, powders, capsules, pills, cachets, and lozenges can be used as solid forms suitable for oral administration.

Liquid form preparations include solutions, suspensions, and emulsions, for example, water or water-propylene glycol solutions. For example, parenteral injection liquid preparations can be formulated as solutions in aqueous polyethylene glycol solution.

Sterile liquid form compositions include sterile solutions, suspensions, emulsions, syrups and elixirs. The active ingredient can be dissolved or suspended in a pharmaceutically acceptable carrier, such as sterile water, sterile organic solvent or a mixture of both.

The compound of Formula (I) or a pharmaceutically acceptable salt thereof may be formulated in micelles or liposomes. Liposomes are unilamellar or multilamellar vesicles which have a membrane formed from a lipophilic material and an aqueous interior. As the compound of formula (I) comprises a steroid portion, the compound may be incorporated, at least in part, into the lipophilic portion of the liposome. Liposome design may include, for example, opsonins or ligands in order to improve the attachment of liposomes to tissue or to activate events such as, for example, endocytosis.

The formation of liposomes may depend on the physicochemical characteristics such as the agent and the liposomal ingredients, the nature of the medium in which the lipid vesicles are dispersed, the effective concentration of the agent, any additional processes involved during the application and/or delivery of the vesicles, the optimisation size, polydispersity and the shelf-life of the vesicles for the intended application, and the batch-to-batch reproducibility and possibility of large-scale production of safe and efficient liposomal products.

Methods for the production of liposomes and micelles for delivery of drugs are known in the art.

The pharmaceutical compositions according to the present invention may be formulated for parenteral administration (e. g. by injection, for example bolus injection or continuous infusion) and may be presented in unit dose form in ampoules, pre-filled syringes, small volume infusion or in mufti-dose containers with an added preservative. The pharmaceutical compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilisation from solution, for constitution with a suitable vehicle, e.g. sterile, pyrogen-free water, before use.

Pharmaceutical forms suitable for injectable use include sterile injectable solutions or dispersions, and sterile powders for the extemporaneous preparation of sterile injectable solutions. They should be stable under the conditions of manufacture and storage and may be preserved against oxidation and the contaminating action of microorganisms such as bacteria or fungi.

The solvent or dispersion medium for the injectable solution or dispersion may contain any of the conventional solvent or carrier systems for injectable solutions or dispersions, and may contain, for example, water, ethanol, polyol (for example, glycerol, propylene glycol and liquid polyethylene glycol, and the like), suitable mixtures thereof, and vegetable oils.

Pharmaceutical forms suitable for injectable use may be delivered by any appropriate route including intravenous, intramuscular, intracerebral, intrathecal, epidural injection or infusion.

Sterile injectable solutions are prepared by incorporating the active ingredient in the required amount in the appropriate solvent with various other ingredients such as those enumerated above, as required, followed by filtered sterilisation. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, preferred methods of preparation are vacuum drying or freeze-drying of a previously sterile-filtered solution of the active ingredient plus any additional desired ingredients.

The compounds of the invention may be formulated into compositions suitable for oral administration, for example, with an inert diluent or with an assimilable edible carrier, or enclosed in hard or soft shell gelatine capsule, or compressed into tablets, or incorporated directly with the food of the diet. For oral therapeutic administration, the active compound may be incorporated with excipients and used in the form of ingestible tablets, buccal tablets, troches, capsules, elixirs, suspensions, syrups, wafers, and the like.

The amount of active compound in therapeutically useful compositions should be sufficient that a suitable dosage will be obtained.

The tablets, troches, pills, capsules and the like may also contain the components as listed hereafter: a binder such as gum, acacia, corn starch or gelatine; excipients such as dicalcium phosphate; a disintegrating agent such as corn starch, potato starch, alginic acid and the like; a lubricant such as magnesium stearate; and a sweetening agent such a sucrose, lactose or saccharin may be added or a flavouring agent such as peppermint, oil of wintergreen, or cherry flavouring. When the dosage unit form is a capsule, it may contain, in addition to materials of the above type, a liquid carrier.

Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active ingredient(s) may be incorporated into sustained-release preparations and formulations, including those that allow specific delivery of the active ingredient to specific regions of the gut.

Aqueous solutions suitable for oral use can be prepared by dissolving the active component in water and adding suitable colorants, flavours, stabilising and thickening agents, as desired. Aqueous suspensions suitable for oral use can be made by dispersing the finely divided active component in water with viscous material, such as natural or synthetic gums, resins, methylcellulose, sodium carboxymethylcellulose, or other well-known suspending agents.

Pharmaceutically acceptable carriers include any and all pharmaceutically acceptable solvents, dispersion media, coatings, anti-bacterial and anti-fungal agents, isotonic and absorption delaying agents and the like.

Also included are solid form preparations that are intended to be converted, shortly before use, to liquid form preparations for oral administration. Such liquid forms include solutions, suspensions, and emulsions. These preparations may contain, in addition to the active component, colorants, flavours, stabilisers, buffers, artificial and natural sweeteners, dispersants, thickeners, solubilising agents, and the like.

For topical administration to the epidermis, the compounds of the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or colouring agents.

Formulations suitable for topical administration in the mouth include lozenges comprising active agent in a flavoured base, usually sucrose and acacia or tragacanth; pastilles comprising the active ingredient in an inert base such as gelatine and glycerine or sucrose and acacia; and mouthwashes comprising the active ingredient in a suitable liquid carrier.

Solutions or suspensions for nasal administration may be applied directly to the nasal cavity by conventional means, for example with a dropper, pipette or spray. The formulations may be provided in single or multidose form. In the case of a dropper or pipette, this may be achieved by the patient administering an appropriate, predetermined volume of the solution or suspension. In the case of a spray, this may be achieved for example by means of a metering atomising spray pump. To improve nasal delivery and retention the compounds of the invention may be encapsulated with cyclodextrins, or formulated with other agents expected to enhance delivery and retention in the nasal mucosa.

Administration to the respiratory tract may also be achieved by means of an aerosol formulation in which the active ingredient is provided in a pressurised pack with a suitable propellant such as a chlorofluorocarbon (CFC) for example dichlorodifluoromethane, trichlorofluoromethane, or dichlorotetrafluoroethane, carbon dioxide, or other suitable gas.

The aerosol may conveniently also contain a surfactant such as lecithin. The dose of the active ingredient may be controlled by provision of a metered valve.

Alternatively, the active ingredients may be provided in the form of a dry powder, for example a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g. gelatine, or blister packs from which the powder may be administered by means of an inhaler.

In formulations intended for administration to the respiratory tract, including intranasal formulations, the active ingredient will generally have a small particle size for example of the order of 5 to 10 microns or less. Such a particle size may be obtained by means known in the art, for example by micronisation.

When desired, formulations adapted to give sustained release of the active ingredient may be employed.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

It is especially advantageous to formulate parenteral compositions in dosage unit form for ease of administration and uniformity of dosage. Parental compositions may be in the form of physically discrete units suited as unitary dosages for the subjects to be treated, each unit containing a predetermined quantity of the active ingredient calculated to produce the desired therapeutic effect in association a pharmaceutical carrier.

The compounds may also be administered in the absence of carrier where the compounds are in unit dosage form.

Compositions comprising compounds of the invention formulated for oral delivery either alone or in combination with another agent are particularly preferred.

As such, in one embodiment there is provided a pharmaceutical composition for oral administration comprising a compound of Formula (I), or a pharmaceutically acceptable salt thereof, a pharmaceutically acceptable carrier and optionally another agent or further active agent. In one embodiment, the pharmaceutical composition is orally administered in an effective amount to a subject in need of treatment for a parasite infection or disease, for example an apicomplexan parasite infection.

The invention also provides use of a compound of Formula (I) or a pharmaceutically acceptable salt thereof in the manufacture of a medicament for the treatment or prevention of a disease or condition associated with a parasite infection, such as apicomplexan parasite infections.

### Dosages

The term “effective amount” refers to the amount of a compound effective to achieve the desired response, for example, to treat or prevent an apicomplexan parasite infection, or to prevent, reduce or inhibit biofilm formation on a surface.

An appropriate dosage level of a compound of Formula (I), or a pharmaceutically acceptable salt thereof, administered to a subject to treat or prevent an apicomplexan parasite infection will generally be about 0.01 to 500 mg per kg subject body weight per day which can be administered in single or multiple doses.

It will be understood that the specific dose level and frequency of dosage for any particular subject may be varied and will depend upon a variety of factors including the activity of the specific compound employed, the metabolic stability and length of action of that compound, the age, body weight, general health, sex, diet, and genetics of the subject, the mode and time of administration, rate of excretion, drug combinations, and the severity of the particular condition.

Suitable dosages of the compound of Formula (I) or further active agents administered in combination with compound of Formula (I) can be readily determined by a person skilled in the art having regard to the particular compound of Formula (I) or further active agent selected.

It will further be understood that when the compounds of the invention are to be administered in combination with one or more agents, or other active agents, the dosage forms and levels may be formulated for either concurrent, sequential or separate administration or a combination thereof.

### EXAMPLES

The present invention is further described below by reference to the following non-limiting Examples.

### Example 1: Compound List

| Series, Name | Structure |
|---|---|
| 1 | To investigate linking primaquine from the C17 steroidal carbon |
| | via an ‘ether-amide’ linker. DHEA was used as the starting |
| | material due to its structural similarity to cholesterol. |
| 3β-Hydroxy-17,17- (methylenedioxyphenol)- androst-5-ene | 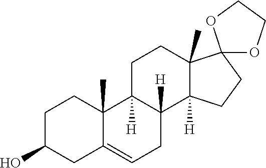 |
| 3β-(Tert- butyldimethylsilyloxy)- 17,17-(ethylenedioxy)- androst-5-ene | 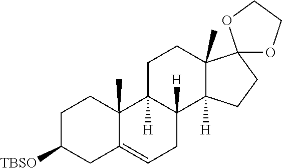 |
| 3β-(Tert- butyldimethylsilyloxy)-17β- (2-hydroxyethoxy)- androst-5-ene | 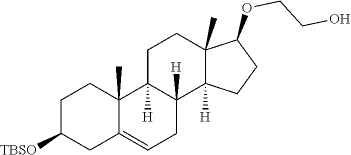 |
| 2-(3β-(Tert- butyldimethylsilyloxy)- androst-5-en-17β-yloxy)- acetic acid | 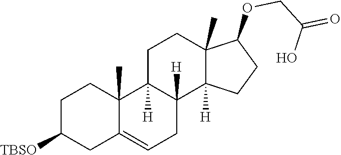 |
| 2-(3β-Hydroxyandrost-5- en-17β-yloxy)-acetate- primaquine amide | 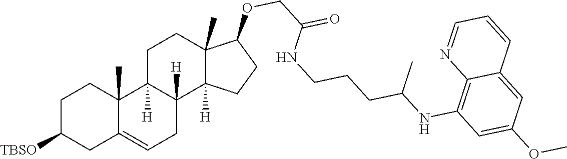 |
| 2 | To investigate variation of B-ring saturation on ether-amide' |
| | linked primaquine from the C17 steroidal carbon |
| 3β-Hydroxy-17,17- (ethylenedioxy)-5α- androstane | 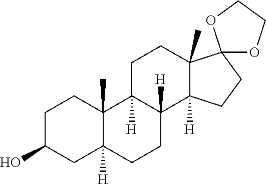 |
| 3β-(Tert- butyldimethylsilyloxy)- 17,17-(ethylenedioxy)-5α- androstane | 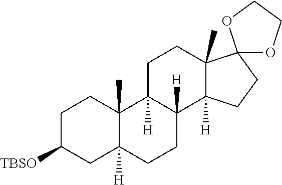 |
| 3β-(Tert- butyldimethylsilyloxy)-17β- (2-hydroxyethoxy)-5α- androstane | 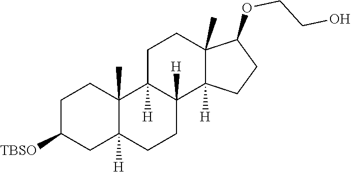 |
| 2-(3β-(Tert- butyldimethylsilyloxy)-5α- androstan-17β-yloxy)- acetic acid | 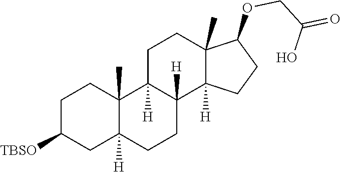 |
| 2-(3β-Hydroxy-5α- androstan-17β-yloxy)- acetate-primaquine amide | 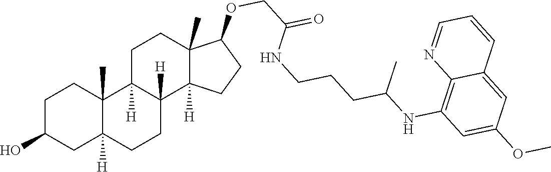 |
| 3 | To investigate the effect of linking the primaquine as an N-alkyl |
| | substituent rather than an amide. This conjugate would likely |
| | require further metabolism to facilitate the release of free drug |
| | compared to previous ether-amide linked conjugates. |
| 2-(3β-(Tert- butyldimethylsilyloxy)- androst-5-en-17β-yloxy)- acetaldehyde | 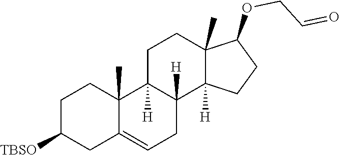 |
| 2-(3β-(Tert- butyldimethylsilyloxy)-5α- androstan-17β-yloxy)- acetaldehyde | 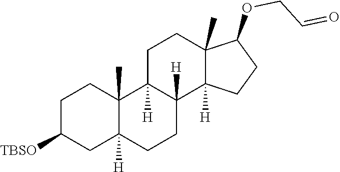 |
| 2-(3β-Hydroxy-5α- androstan-17β-yloxy)- ethyl-primaquine | 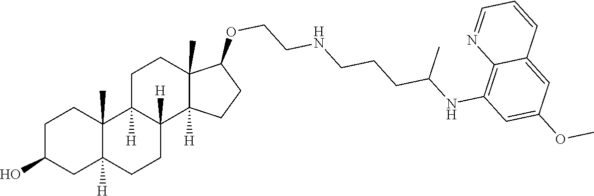 |
| 4 | To investigate linking primaquine from the C17 steroidal carbon |
| | via a ‘succinate’ linker. This conjugate would likely be more |
| | readily cleaved compared to previous ether-amide linked |
| | conjugates. Diester compounds used as experimental controls. |
| 3β-(Tert- butyldimethylsilyloxy)- androst-5-en-17-one | 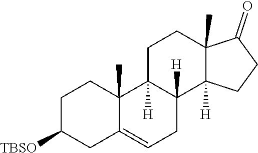 |
| 3β-(Tert- butyldimethylsilyloxy)- androst-5-en-17β-ol | 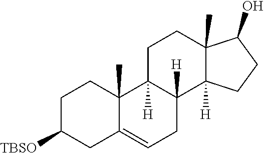 |
| 3β-(Tert- butyldimethylsilyloxy)- androst-5-en-17β-yl hemisuccinate | 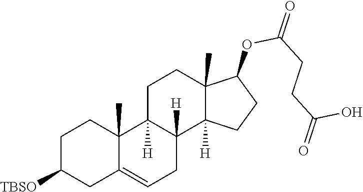 |
| 3β-(Tert- butyldimethylsilyloxy)- androst-5-en-17β-yl succinate-primaquine amide | 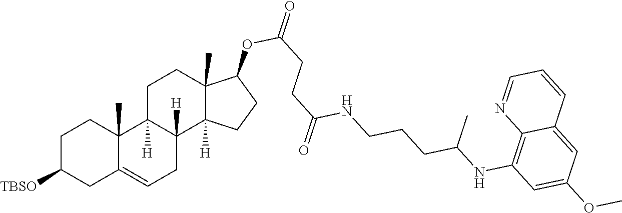 |
| 3β-Hydroxyandrost-5-en- 17β-yl succinate- primaquine amide (C-17-prim; BC5B) | 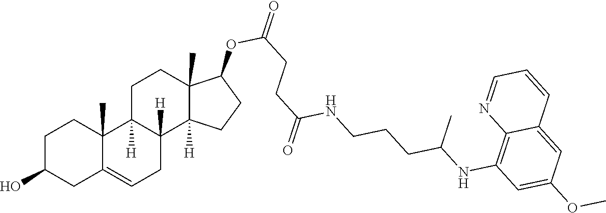 |
| 3β-(Tert- butyldimethylsilyloxy)- androst-5-en-17β-yl succinate-propan-2-yl diester | 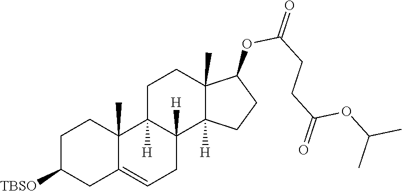 |
| 3β-Hydroxyandrost-5-en- 17β-yl succinate-propan-2- yl diester (C-17-link) | 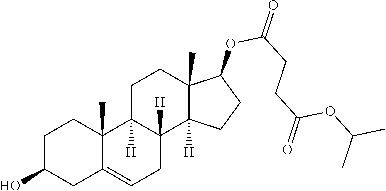 |
| 5 | To investigate more saturated 3β-OH, 5α configuration in |
| | steroid A,B-rings and any effect on transport. Diester |
| | compounds used as experimental controls. |
| 3β-(Tert- butyldimethylsilyloxy)-5α- androstan-17-one | 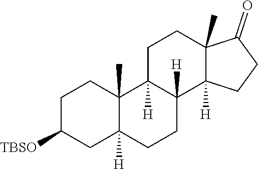 |
| 3β-(Tert- butyldimethylsilyloxy)-5α- androstan-17β-ol | 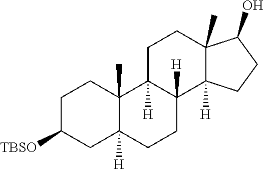 |
| 3β-(Tert- butyldimethylsilyloxy)-5α- androstan-17β-yl hemisuccinate | 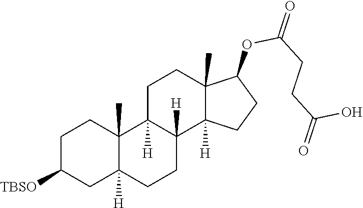 |
| 3β-(Tert- butyldimethylsilyloxy)-5α- androstan-17β-yl succinate-primaquine amide | 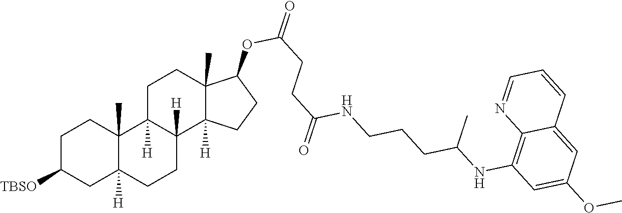 |
| 3β-Hydroxy-5α-androstan- 17β-yl succinate- primaquine amide | 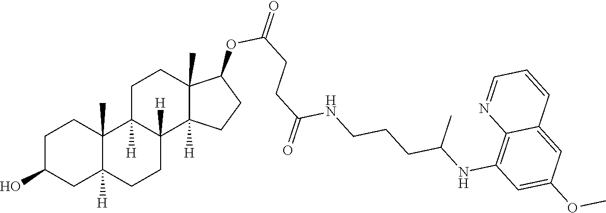 |
| 3β-(Tert- butyldimethylsilyloxy)-5α- androstan-17β-yl succinate-propan-2-yl diester | 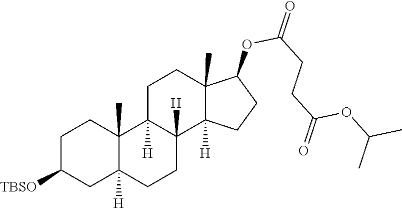 |
| 3β-Hydroxy-5α-androstan- 17β-yl succinate-propan-2- yl diester | 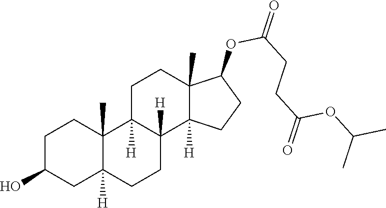 |
| 6 | To investigate more saturated 3α-OH, 5α configuration in |
| | steroid A,B-rings and any effect on transport. Diester |
| | compounds used as experimental controls. |
| 3α-(Tert- butyldimethylsilyloxy)-5α- androstan-17-one | 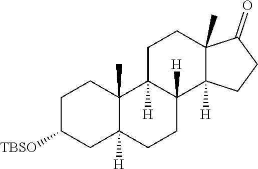 |
| 3α-(Tert- butyldimethylsilyloxy)-5α- androstan-17β-ol | 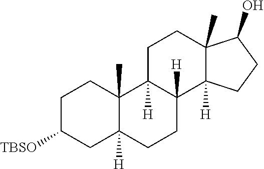 |
| 3α-(Tert- butyldimethylsilyloxy)-5α- androstan-17β-yl hemisuccinate | 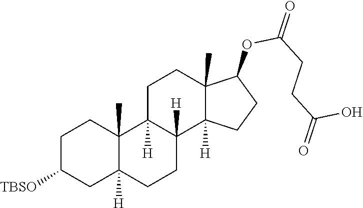 |
| 3α-(Tert- butyldimethylsilyloxy)-5α- androstan-17β-yl succinate-primaquine amide | 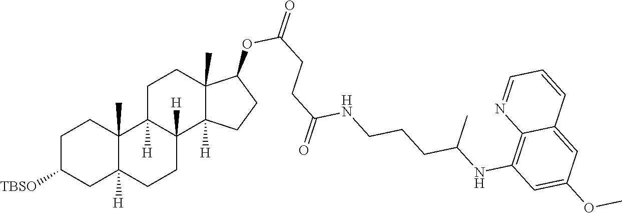 |
| 3α-Hydroxy-5α-androstan- 17β-yl succinate- primaquine amide | 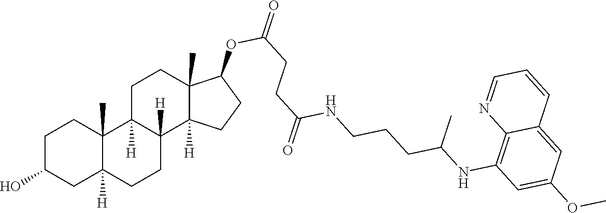 |
| 3α-(Tert- butyldimethylsilyloxy)-5α- androstan-17β-yl succinate-propan-2-yl diester | 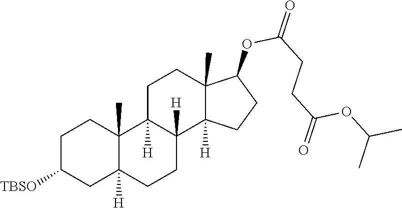 |
| 3α-Hydroxy-5α-androstan- 17β-yl succinate-propan-2- yl diester | 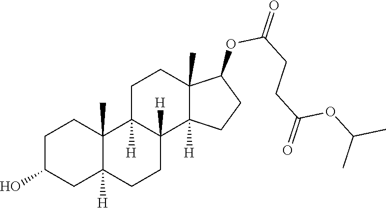 |
| 7 | To investigate more saturated 3α-OH, 5β configuration in |
| | steroid A,B-rings and any effect on transport. Diester |
| | compounds used as experimental controls. |
| 3α-(Tert- butyldimethylsilyloxy)-5β- androstan-17-one | 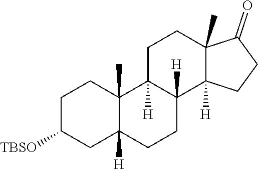 |
| 3α-(Tert- butyldimethylsilyloxy)-5β- androstan-17β-ol | 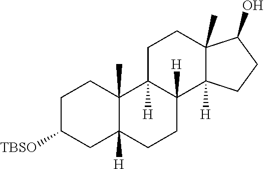 |
| 3α-(Tert- butyldimethylsilyloxy)-5β- androstan-17β-yl hemisuccinate | 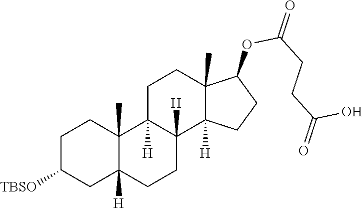 |
| 3α-(Tert- butyldimethylsilyloxy)-5β- androstan-17β-yl succinate-primaquine amide | 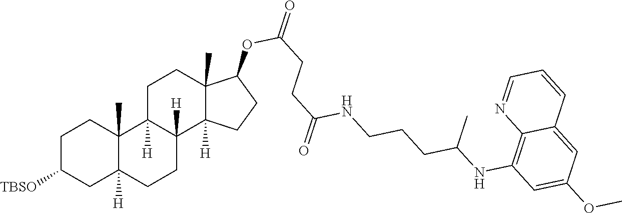 |
| 3α-Hydroxy-5β-androstan- 17β-yl succinate- primaquine amide | 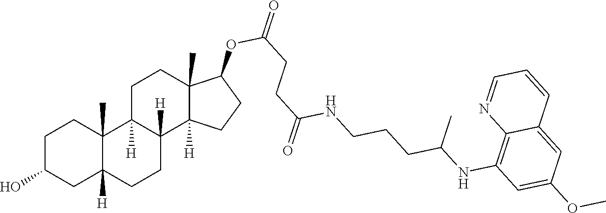 |
| 3α-(Tert- butyldimethylsilyloxy)-5β- androstan-17β-yl succinate-propan-2-yl diester | 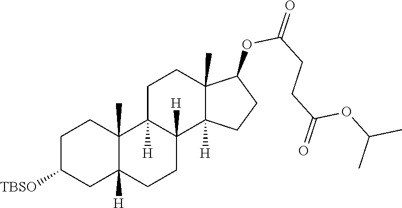 |
| 3α-Hydroxy-5β-androstan- 17β-yl succinate-propan-2- yl diester | 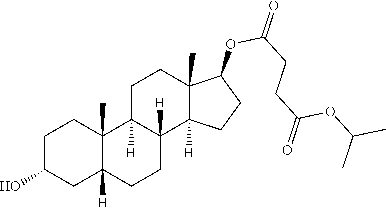 |
| 8 | To investigate clinking primaquine from the steroidal C3 carbon |
| | of DHEA via a ‘succinate’ linker and any effect on transport. |
| | Diester compounds used as experimental controls. |
| Androst-5-en-17-one-3β-yl hemisuccinate | 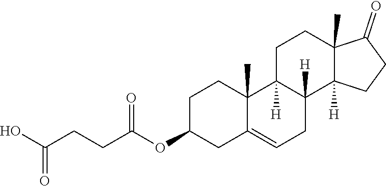 |
| Androst-5-en-17-one-3β-yl succinate-primaquine amide | 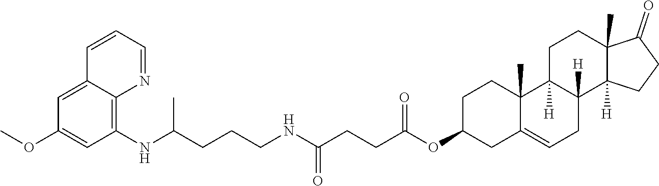 |
| Androst-5-en-17-one-3β-yl succinate-propan-2-yl diester | 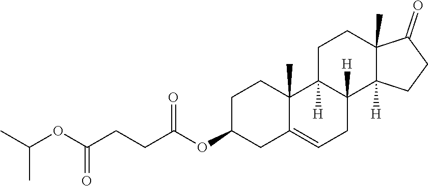 |
| 9 | To investigate the importance of an alcohol at the C3 position |
| | using a 3-one steroid derivative as the steroid starting material |
| | and any effect on transport. Diester compounds used as |
| | experimental controls. |
| Testosterone-17β-yl hemisuccinate | 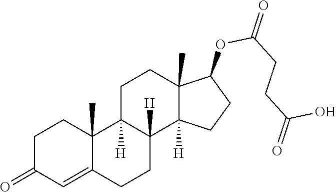 |
| Testosterone-17β-yl succinate-primaquine amide | 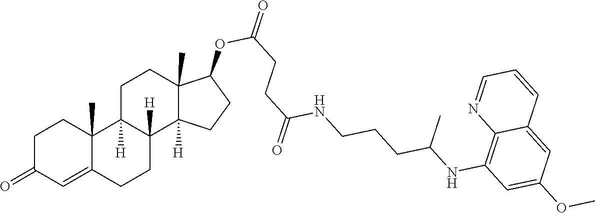 |
| Testosterone-17β-yl succinate-propan-2-yl diester | 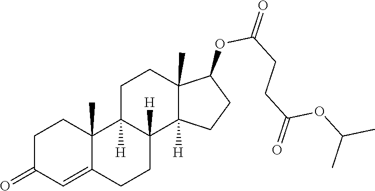 |
| 10 | Control compounds for testing of succinate and ether-amide |
| | linked primaquine conjugates. |
| Propan-2-yl hemisuccinate | 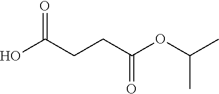 |
| Propan-2-yl succinate- primaquine amide (Prim-link) | 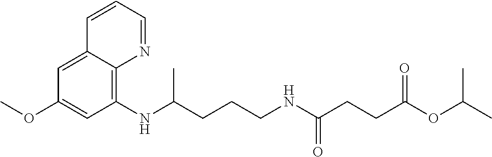 [SMILES: COc1cc(NC(C)CCCNC(=O)CCC(=O)OC(C)C)c2ncccc2c1] |
| 2-Methoxyacetate- primaquine amide | 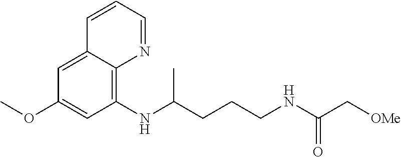 [SMILES: COCC(=O)NCCCC(C)Nc1cc(OC)cc2cccnc12] |
| 11 | Novel dialkyl peroxide containing cholesterol derivative. |
| 2-(3β-(Tert- butyldimethylsilyloxy)- androst-5-en-17β-yloxy)- ethyl para- toluenesulfonate | 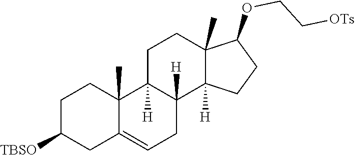 |
| 2-(3β-(Tert- butyldimethylsilyloxy)-5α- androstan-17β-yloxy)-ethyl para-toluenesulfonate | 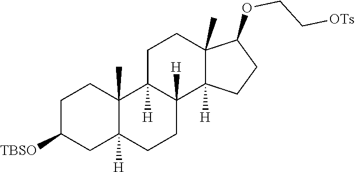 |
| 17β-(2-(Tert-butylperoxy)- ethoxy)-5α-androstan-3β- ol | 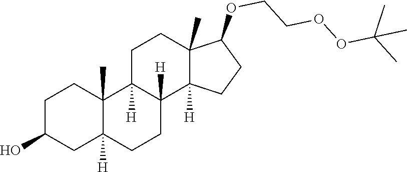 |
| 12 | To investigate the effect of conjugating known anti-malarial |
| | drug, artesunate, to both the 3-OH of cholesterol and the 17-OH |
| | position of a DHEA-derived cholesterol analogue. |
| Cholesterol hemisuccinate | 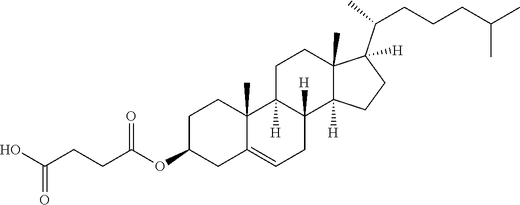 |
| 3β-Succinate-(propan-2-yl diester)-cholest-5-ene (C-3-link) | 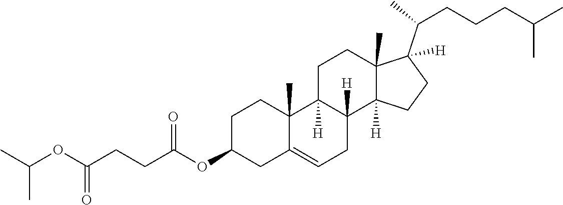 |
| 3β-Artesunate ester- cholest-5-ene | 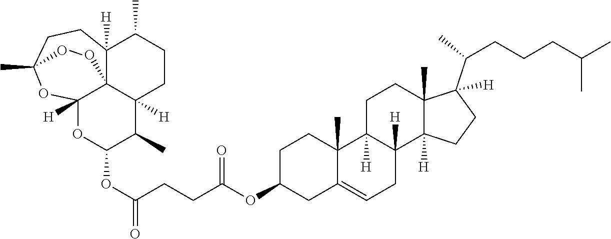 |
| Propan-2-yl artesunate ester (Art-link) | 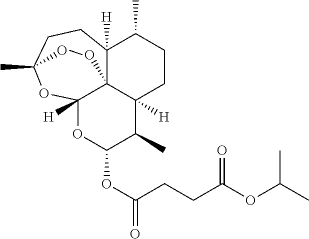 [SMILES: CC(C)OC(=O)CCC(=O)O[C@@H]1O[C@@H]2O[C@]3(C)CC[C@H]4[C@H](C)CC[C@@H]([C@H]1C)[C@@]24OO3] |
| 3β-(tert- butyldimethylsilyloxy)- androst-5-en-17β-yl succinate-artesunate ester | 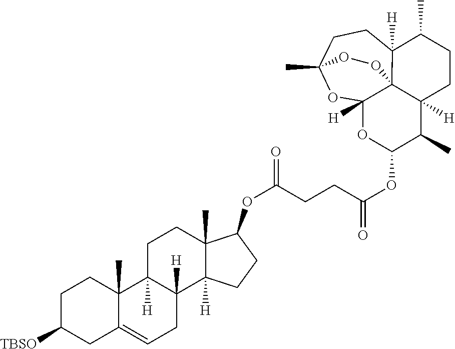 |
| 3β-Hydroxyandrost-5-en- 17β-yl succinate- artesunate ester (C-17-art) | 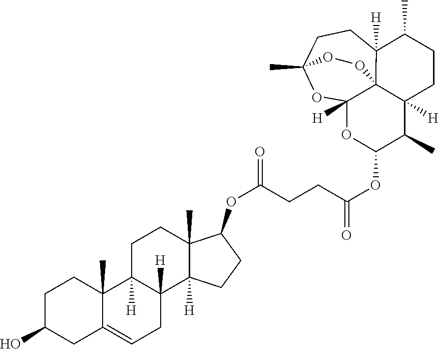 |
| 13 | To investigate the mode of drug entry and cleavage in cells by |
| | conjugation of a fluorescent dye to a DHEA-derived cholesterol |
| | analogue |
| 3β-(tert- butyldimethylsilyloxy)- androst-5-en-17β-yl succinate-chloromethyl ester | 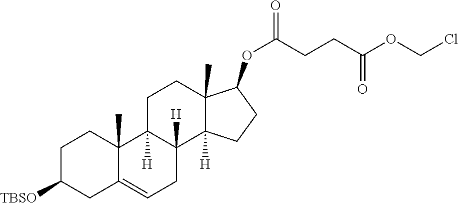 |
| 3β-(tert- butyldimethylsilyloxy)- androst-5-en-17β-yl succinate- (hydroxymethoxy)- resorufin ester | 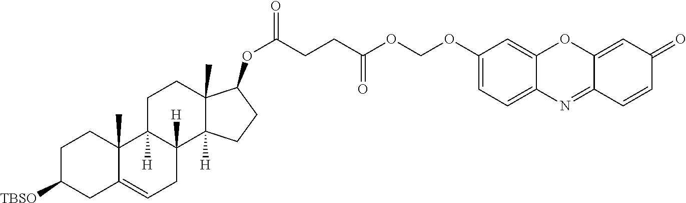 |
| 3β-Hydroxyandrost-5-en- 17β-yl succinate- (hydroxymethoxy)- resorufin ester | 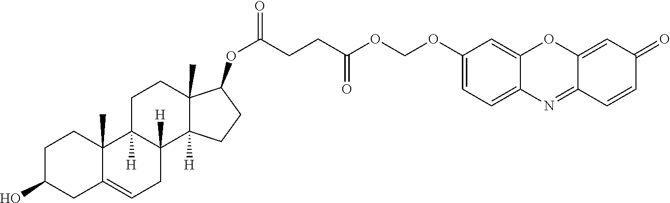 |

### 1.1 Experimental

Unless otherwise specified ¹H NMR and ¹³C NMR experiments were performed using deuterated chloroform (CDCl₃) or deuterated methanol (MeOD-d₄) using Bruker Avance 400 MHz, 600 MHz or 700 MHz spectrometers at 298K. Deuterated solvents supplied by Cambridge Isotope Laboratories, Inc. Residual solvent peaks or ¹³C signals corresponding to CHCl₃ and MeDH₂OD-d₃ were used as reference peaks corresponding to values given by Fulmer et al., (2010).^{1A} Analysis of these spectra was completed using MestReNOVA. Chemical shifts are reported in parts per million (ppm). Multiplicity is assigned as s=singlet, d=doublet, t=triplet, q=quartet, sept=septet and m=multiplet or combination of these. Where compounds are found as a pair of inseparable diastereomers; peaks corresponding to the same ¹H and ¹³C environments are given as peak 1/peak 2. Coupling constants (J) reported in Hz.

Unless otherwise stated, low resolution mass spectrometry (LRMS) was performed using a Waters LCT Premier XE mass spectrometer and high resolution mass spectrometry (HRMS) was performed using a Waters SYNAPT G2-Si mass spectrometer. Samples were prepared at a concentration of ˜1 milligram of analyte in 1 milliliter of methanol for LRMS, and was subsequently diluted in methanol for HRMS. Infrared spectra were recorded using Perkin-Elmer 1800 Series FTIR spectrometer. Specific rotation recorded using the Rudolf research systems Autopol I polarimeter where 10 milligrams of analyte was dissolved in 1.0 millilitres of chloroform. Thin layer chromatography (TLC) analysis performed using Merck TLC silica gel 60 F254 plates using mobile phases as stated.

Purification by silica flash chromatography was conducted using chem-supply silica gel 60 0.04-0.06 mm (230-400 mesh ASTM) using eluent as stated. Purification by high performance liquid chromatography (HPLC) was conducted using Waters 2695 separations module, Agilent Pursuit XRs 5 C18 250×10 mm column, Waters 2998 Photodiode array detector (266 nm) and Waters Fraction Collector Ill, controlled by Waters Empower 2 software. Purity of compounds used for biological testing was determined using the same Waters separations module and photodiode array detector with an Agilent Eclipse XDB-C18 5 μm column. General eluent used: 95% HPLC methanol (Honeywell—Burdick & Jackson): 5% (0.1% HPLC trifluroacetic acid) (Sigma-Aldrich) solution in filtered water. All final conjugates underwent purity checks using this method to ensure >95% purity.

### Abbreviations

TBS tert-butyldimethylsilyl
TES triethylsilyl
THF tetrahydrofuran
TLC thin layer chromatography
DMF N,N-dimethylformamide
DMAP 4-dimethylaminopyridine
DIPEA N,N-diisopropylethylamine
EDC 1-ethyl-3-(-3-dimethylaminopropyl) carbodiimide hydrochloride
Boc tert-butyloxycarbonyl
Ts toluenesulfonyl

### 1.2 General Synthetic Procedures

General procedure A for the preparation of tert-butyldimethylsilyl ether protected steroid alcohols adapted from Yamauchi et al., (2005).^{2A} Tert-butyldimethylsilyl chloride (1.5 equiv.) was added to a stirring solution of steroid (1.0 equiv.) and imidazole (2.5 equiv.) in anhydrous DMF. The reaction mixture was stirred at room temperature until reaction completion by TLC.

General procedure B for the preparation of 17β-hydroxy steroids adapted from Moreira et al., (2008).^{3A} Sodium borohydride (2.0 equiv.) was added to a stirring solution of 17-ketone steroid xx-xx (1.0 equiv.) in ice bath cooled (v/v) 1:1 THF/MeOH. The reaction was brought to room temperature and stirred until reaction completion by TLC.

General procedure C for the preparation of 17β-yl hemisuccinate steroids adapted from Keglevich et al., (2019).^{4A} Succinic anhydride (2.0 equiv.) was added to a stirring solution of 17β-hydroxy steroid (1.0 equiv.), Et₃N (>10 equiv.) and DMAP (2.0 equiv.) in anhydrous toluene. The reaction mixture was brought to reflux and stirred until reaction completion by TLC.

General procedure D for the preparation of amide linked steroid primaquine adapted from Femandes et al., (2009).^{5A} EDC HCl (2.0 equiv.) was added to a stirring solution of 17β-yl hemisuccinate steroid (1.0 equiv.), HOBt (2.0 equiv.) and DIPEA (>10 equiv.) in anhydrous CH₂Ch. Separate to this, primaquine bisphosphate (2.0 equiv.) was added to a solution of DIPEA (>10 equiv.) in CH₂Cl₂ and was stirred until dissolved. After 30 minutes, the two solutions were combined and the reaction mixture was stirred at room temperature for 2 days, or until reaction completion by TLC.

General procedure E for the deprotection of tert-butyldimethylsilyl ether protected steroids to the corresponding alcohols adapted from Yamashita et al., (2005).^{6A} Camphor sulfonic acid (2.0 equiv.) was added to a stirring solution of tert-butyldimethylsilyl ether protected steroid (1.0 equiv.) in (v/v) 1:1 MeOH/CH₂Cl₂. The reaction mixture was stirred at room temperature until reaction completion by TLC.

General procedure F for the preparation of steroid succinate propan-2-yl diesters adapted from Krausova et al., (2018).^{7A} EDC HCl (2.0 equiv.) was added to a stirring solution of propan-2-yl hemisucciante (2.0 equiv.), DMAP (2.0 equiv.) and DIPEA (>10 equiv.) in CH₂Cl₂. After 10-30 minutes steroid alcohol (1.0 equiv.) was added to the solution. The reaction mixture was stirred at room temperature until reaction completion by TLC.

General procedure G for the preparation of succinate ester linked steroid-drug conjugates and controls adapted from Krausova et al., (2018).^{7A} EDC HCl (2.0 equiv.) was added to a stirring solution of 17β-yl hemisuccinate steroid or propan-2-yl hemisucciante (1.0 equiv.), HOBt (0.2 equiv.), DMAP (2.0 equiv.) and DIPEA (>10 equiv.) in anhydrous CH₂Cl₂. After 5-20 minutes drug alcohol (1.0 equiv.) was added to the solution. The reaction mixture was stirred at room temperature until reaction completion by TLC.

### 1.3 Specific Transformations

### 3β-Hydroxy-17,17-(ethylenedioxy)-androst-5-ene

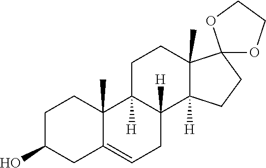

Procedure adapted from Liu, Stuhmiller and McMorris, (1988).^{8A} 3β-Hydroxyandrost-5-en-17-one (1.50 g, 5.20 mmol) was combined with ethylene glycol (6.45 g, 104 mmol), triethylorthoformate (4.62 g, 31 mmol) and para-toluenesulfonic acid (25 mg, 0.13 mmol) in CH₂Cl₂ (4 mL). The reaction was stirred under N₂ at room temperature overnight. The reaction was quenched with Et₃N (30 μL) and diluted with water (30 mL) and CH₂Cl₂ (30 mL). The aqueous layer was further extracted with CH₂Cl₂ (2×30 mL). The combined organic extract was washed with saturated NaCl solution, dried over anhydrous MgSO₄ and filtered. The solvent was removed under reduced pressure and the residue obtained was washed with cold MeOH (10 mL) and purified by silica column chromatography (30% EtOAc:n-hexanes) to yield 3β-Hydroxy-17,17-(ethylenedioxy)-androst-5-ene (xx) (920 mg. 53%) as a white solid.

m.p 165-167° C. (lit.^{9A} 162-165° C.) ¹H NMR (400 MHz, CDCl₃) δ 5.37-5.31 (m, 1H), 3.95-3.83 (m, 4H), 3.52 (m, 1H), 2.31-0.93 (m, 19H), 1.00 (s, 3H), 0.85 (s, 3H), OH not observed; ¹³C NMR (101 MHz, DMSO-d₆) δ 141.3, 120.2, 118.5, 69.9, 64.6, 64.0, 50.2, 49.7, 45.1, 42.2, 36.9, 36.1, 33.7, 31.7, 31.4, 30.8, 30.2, 22.3, 20.1, 19.2, 14.1; LRMS (ESI+) found m/z 355.2 [M+Na]⁺; HRMS (ESI+) found m/z 355.2259 [M+Na]⁺, theoretical m/z 355.2249 [M+Na]⁺; IR 3546, 3140, 2989, 2828 cm⁻¹.

### 3β-(Tert-butyldimethylsilyloxy)-17,17-(ethylenedioxy)-androst-5-ene

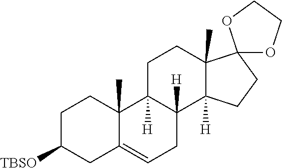

3β-Hydroxy-17,17-(ethylenedioxy)-androst-5-ene (500 mg, 1.50 mmol) in anhydrous DMF (8 mL) was treated as per general procedure A. The reaction mixture was diluted with water (30 mL) and extracted with ethyl acetate (3×20 mL). The combined organic extract was washed with saturated NaCl solution, dried over anhydrous MgSO₄ and filtered. The solvent was removed under reduced pressure and the residue obtained was purified by silica column chromatography (30% EtOAc:n-hexanes) to yield 3β-(tert-butyldimethylsilyloxy)-17,17-(ethylenedixoy)-androst-5-ene (xx) (599 mg, 89%) as a white solid.

m.p 119-121° C. (lit.^{10A} 121-122° C.) ¹H NMR (400 MHz, CDCl₃) δ 5.34-5.28 (m, 1H), 3.96-3.84 (m, 4H), 3.48 (m, 1H), 2.29-0.92 (m, 19H), 1.00 (s, 3H), 0.88 (s, 9H), 0.85 (s, 3H), 0.05 (s, 6H); ¹³C NMR (101 MHz, Dioxane) δ 142.23, 121.66, 119.94, 73.29, 65.67, 65.07, 51.47, 51.13, 46.46, 43.60, 38.07, 37.40, 34.81, 33.02, 32.88, 32.08, 31.43, 26.30, 23.46, 21.26, 19.70, 18.74, 14.62, −4.36 (2C); LRMS (ESI+) found m/z 469.3 [M+Na]⁺, HRMS (ESI+) found m/z 469.3118 [M+Na]⁺, theoretical m/z 469.3114 [M+Na]⁺; IR 2987, 2886 cm⁻¹.

### 3β-(Tert-butyldimethylsilyloxy)-17β-(2-hydroxyethoxy)-androst-5-ene

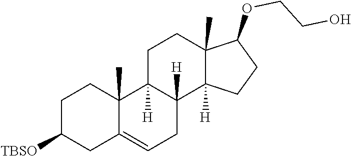

Procedure adapted from Upasani et al., (1999).^{11A} 3β-(Tert-butyldimethylsilyloxy)-17,17-(ethylenedioxy)-androst-5-ene (500 mg, 1.12 mmol) in anhydrous THF (5 mL) was cooled over an ice bath under N₂ atmosphere. AlCl₃ (299 mg, 2.24 mmol) in anhydrous THF (1.5 mL) and LiAlH₄ (85 mg, 2.24 mmol) in anhydrous THF (2 mL) were added individually by dropwise addition to the previous solution with stirring. The reaction was brought to reflux and stirred for 3 hours. The reaction was then cooled over an ice bath and quenched by addition of 10% Rochelle's salt solution (11 mL) and EtOAc (15 mL). The mixture was left stirring for a further 30 minutes. The solution was then diluted with water (20 mL) and the aqueous layer was extracted with EtOAc (3×20 mL). The combined organic extract was washed with saturated NaCl solution, dried over anhydrous MgSO₄ and filtered. The solvent was removed under reduced pressure and the residue obtained was purified by silica column chromatography (30% EtOAc:n-hexanes) to yield 3β-(tert-butyldimethylsilyloxy)-17β-(2-hydroxyethoxy)-androst-5-ene (xx) (339 mg, 68%) as a white solid.

m.p 117-119° C.; ¹H NMR (400 MHz, CDCl₃) δ 5.30 (m, 1H), 3.71-3.66 (m, 2H), 3.61-3.44 (m, 3H), 3.35 (t, J=8.3 Hz, 1H), 2.29-0.91 (m, 19H), 1.00 (s, 3H), 0.88 (s, 9H), 0.77 (s, 3H), 0.05 (s, 6H), OH not observed; ¹³C NMR (101 MHz, CDCl₃) δ 141.8, 121.0, 89.6, 72.7, 71.1, 62.2, 51.8, 50.4, 43.0, 42.9, 38.0, 37.5, 36.8, 32.2, 31.9, 31.7, 28.2, 26.1, 23.5, 20.9, 19.6, 18.4, 11.7, −4.4 (2C); LRMS (ESI+) found m/z 471.3 [M+Na]⁺; HRMS (ESI+) found m/z 471.3284 [M+Na]⁺, theoretical (C₂₇H₄₈O₃SiNa) m/z 471.3270 [M+Na]⁺; IR 3462, 2927, 2856 cm⁻1; specific rotation [α]_{D}²⁵−33.20 (c 1.0, CHCl₃).

### 2-(3β-(Tert-butyldimethylsilyloxy)-androst-5-en-17β-yloxy)-acetic acid

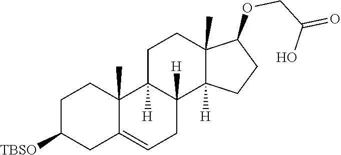

Procedure adapted from Epp and Widlanski, (1999).^{12A} 3β-(Tert-butyldimethylsilyloxy)-17β-(2-hydroxyethoxy)-androst-5-ene (50 mg, 0.11 mmol), TEMPO (4 mg, 0.025 mmol) and bis(acetoxy)iodobenzene (90 mg, 0.28 mmol) were combined in (v/v) 1:1 CH₂Cl₂/H₂O (4 mL). The reaction was vigorously stirred at room temperature for 2 hours. The reaction was quenched with saturated Na₂S₂O₃ solution (1 mL), diluted with water (20 mL), and brought to pH 3 with additions of 5% citric acid solution. The aqueous layer was extracted with CHCl₃ (2×20 mL) and the combined organic extract was washed with saturated NaCl solution, dried over anhydrous MgSO₄ and filtered. The solvent was removed under reduced pressure and the residue obtained was purified by silica column chromatography (2% acetic acid:20% EtOAc:n-hexanes) to yield 2-(3β-(tert-butyldimethylsilyloxy)-androst-5-en-17β-yloxy)-acetic acid (xx) (35 mg, 68%) as a white oily solid.

¹H NMR (400 MHz, CDCl₃) δ 5.30 (m, 1H), 4.13 (m, 2H), 3.54-3.41 (m, 2H), 2.32-0.92 (m, 19H), 1.01 (s, 3H), 0.89 (s, 9H), 0.81 (s, 3H), 0.05 (s, 6H); ¹³C NMR (101 MHz, DMSO-d₆) δ 171.7, 140.8, 120.8, 88.5, 72.0, 66.8, 50.7, 49.7, 42.5, 42.3, 37.0, 36.8, 36.1, 31.8, 31.3, 30.9, 27.1, 25.8, 22.9, 20.3, 19.1, 17.8, 11.4, −4.6 (2C); LRMS (ESI−) found m/z 461.3 [M−H]⁻; HRMS (ESI−) found m/z 461.3088 [M−H]⁻, theoretical (C₂₇H₄₅O₄Si) m/z 461.3087 [M−H]⁻.

### 2-(3β-Hydroxyandrost-5-en-17β-yloxy)-acetate-primaquine amide

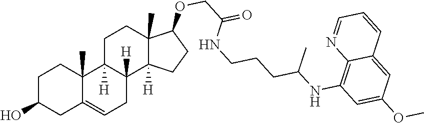

2-(3β-(Tert-butyldimethylsilyloxy)-androst-5-en-17β-yloxy)-acetic acid (30 mg, 0.065 mmol in CH₂Cl₂ was treated as per general procedure D for 2 days. The reaction mixture was diluted with water (20 mL) and 5% citric acid solution until pH 5. The aqueous layer was further extracted with CH₂Cl₂ (2×20 mL). The combined organic extract was washed with saturated NaHCO₃ solution and saturated NaCl solution, dried over anhydrous MgSO₄ and filtered. The solvent was removed under reduced pressure and the residue obtained was purified by silica column chromatography (50% diethyl ether:CH₂Cl₂) to yield 2-(3β-(tert-butyldimethylsilyloxy)androst-5-en-17β-yloxy)acetate-primaquine amide (31.7 mg, 70%) that was used without further purification in the next step. 2-(3β-(tert-butyldimethylsilyloxy)-androst-5-en-17β-yloxy)-acetate-primaquine amide in MeOH/CH₂Cl₂ (2 mL) was treated as per general procedure E for 30 minutes. The reaction was quenched with saturated NaHCO₃ and diluted with water (10 mL). The aqueous layer was extracted with CH₂Cl₂ (2×20 mL). The combined organic extract was washed with saturated NaCl solution, dried over anhydrous MgSO₄ and filtered. The solvent was removed under reduced pressure and the residue obtained was purified by silica column chromatography (8% MeOH:CH₂Cl₂) to yield 2-(3β-hydroxyandrost-5-en-17β-yloxy)-acetate-primaquine amide (xx) (21 mg, 67% over 2 steps) as a green oil.

¹H NMR (400 MHz, MeOD) δ 8.53-8.45 (m, 1H), 8.01 (d, J=8.3 Hz, 1H), 7.40-7.30 (m, 1H), 6.44 (s, 1H), 6.34-6.26 (m, 1H), 5.34-5.27 (m, 1H), 3.90 (s, 2H), 3.87 (s, 3H), 3.70-3.59 (m, 1H), 3.43-3.37 (m, 1H), 3.31-3.19 (m, 3H), 2.24-0.78 (m, 23H), 1.31/1.28 (m, 3H), 0.98/0.95 (m, 3H), 0.73 (s, 3H), OH, aniline NH and amide NH not observed; ¹³C NMR (101 MHz, MeOD) δ 172.96/172.93, 160.9, 146.1, 145.3, 142.24/142.20, 136.5, 136.40/136.34, 131.62/131.60, 122.97/122.94, 122.1, 98.3, 93.1, 91.23/91.17, 72.3, 70.25/70.11, 55.6, 52.48/52.39, 51.59/51.50, 48.9, 43.92/43.86, 43.0, 39.8, 38.76/38.65, 38.46/38.42, 37.66/37.60, 34.84/34.81, 32.97/32.91, 32.44/32.39, 32.3, 28.5, 27.0, 24.31/24.28, 21.75/21.71, 20.66/20.63, 19.88/19.86, 11.93/11.91; LRMS (ESI+) found m/z 590.4 [M+H]⁺; HRMS (ESI+) found m/z 590.3959 [M+H]⁺, theoretical (C₃₈H₅₂N₃O₅) m/z 590.3958 [M+H]⁺; IR 3391, 2930, 2851, 1661, 1615, 1594, 1576, 1519 cm⁻¹.

### 3β-Hydroxy-17,17-(ethylenedioxy)-5α-androstane

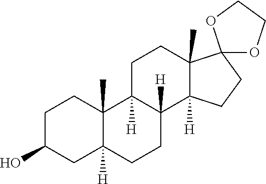

Procedure adapted from Liu, Stuhmiller and McMorris, (1988).^{8A} 3β-Hydroxy-5α-androstan-17-one (1.50 g, 5.16 mmol) was combined with ethylene glycol (6.41 g, 103 mmol), triethylorthoformate (4.59 g, 30.9 mmol) and p-toluenesulfonic acid (25 mg, 0.13 mmol) in CH₂Cl₂ (4 mL). The reaction was stirred under N₂ at room temperature overnight. The reaction was quenched with Et₃N (30 μL) and diluted with water (30 mL) and CH₂Cl₂ (30 mL). The aqueous layer was further extracted with CH₂Cl₂ (2×30 mL). The combined organic extract was washed with saturated NaCl solution, dried over anhydrous MgSO₄ and filtered. The solvent was removed under reduced pressure and the residue obtained was washed with cold MeOH (10 mL) and purified by silica column chromatography (30% EtOAc:n-hexanes) to yield 3β-hydroxy-17,17-(ethylenedioxy)-5α-androstane (1.20 g, 70%), as a white solid.

m.p 152-154° C. (lit.^{13A} 152-155° C.); ¹H NMR (400 MHz, CDCl₃) δ 3.95-3.80 (m, 4H), 3.58 (m, 1H), 1.98-0.85 (m, 21H), 0.82 (s, 3H), 0.79 (s, 3H), 0.70-0.62 (m, 1H), OH not observed; ¹³C NMR (101 MHz, Dioxane-d₈) δ 119.9, 71.0, 65.7, 65.1, 55.2, 51.1, 46.7, 45.8, 39.4, 38.0, 36.7, 36.4, 34.8, 32.5, 32.3, 31.5, 29.5, 23.4, 21.4, 14.8, 12.7. LRMS (ESI+) found m/z 335.5 [M+H]⁺; HRMS (ESI+) found m/z 335.2583 [M+H]⁺, theoretical m/z 355.2586 [M+H]⁺; IR 3369, 3100, 2950, 2859 cm⁻¹.

### 3β-(Tert-butyldimethylsilyloxy)-17,17-ethylenedioxy)-5α-androstane

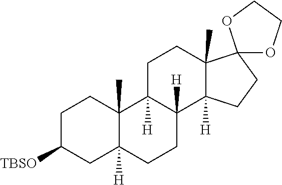

3β-Hydroxy-17,17-(ethylenedioxy)-5α-androstane (500 mg, 1.50 mmol in anhydrous DMF (10 mL) was treated as per general procedure A. The reaction mixture was diluted with water (30 mL) and extracted with ethyl acetate (3×20 mL). The combined organic extract was washed with saturated NaCl solution, dried over anhydrous MgSO₄ and filtered. The solvent was removed under reduced pressure and the residue obtained was purified by silica column chromatography (30% EtOAc:n-hexanes) to yield 3β-(tert-butyldimethylsilyloxy)-17,17-(ethylenedioxy)-5α-androstane (607 mg, 90%) as a white solid.

m.p 137-139° C.; ¹H NMR (400 MHz, Dioxane-d₈) δ 3.85-3.71 (m, 4H), 3.58 (m, 1H), 1.92-0.90 (m, 21H), 0.86 (s, 9H), 0.81 (s, 3H), 0.79 (s, 3H), 0.63 (m, 1H), 0.03 (s, 6H); ¹³C NMR (101 MHz, Dioxane-d₈) δ 119.9, 72.7, 65.7, 65.1, 55.2, 51.1, 46.7, 45.8, 39.6, 37.9, 36.6, 36.3, 34.8, 32.8, 32.3, 31.5, 29.4, 26.3, 23.4, 21.4, 18.7, 14.8, 12.6, −4.3 (2C); LRMS (ESI+) found m/z 449.3 [M+H]⁺; HRMS (ESI+) found m/z 471.3289 [M+Na]⁺, theoretical (C₂₇H₄₈O₃SiNa) m/z 471.3270; IR 2929, 2857 cm⁻¹; specific rotation [α]_{D}²⁵−14.70 (c 1.0, CHCl₃).

### 3β-(Tet-butyldimethylsilyloxy)-17β-(2-hydroxyethoxy)-5α-androstane

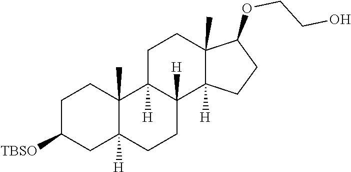

Procedure adapted from Upasani et al., (2004)^{11A}. 3β-(Tert-butyldimethylsilyloxy)-17,17-(ethylenedioxy)-5α-androstane (500 mg, 1.12 mmol) in anhydrous THF (5 mL) was cooled over an ice bath under N₂ atmosphere. AlCl₃ (300 mg, 2.24 mmol) in anhydrous THF (1.5 mL) and LiAlH₄ (85 mg, 2.24 mmol) in anhydrous THF (2 mL) were added individually by dropwise addition to the previous solution with stirring. The reaction was brought to reflux and stirred for 2 hours. The reaction was then cooled over an ice bath and quenched by addition of 10% Rochelle's salt solution (11 mL) and EtOAc (15 mL). The mixture was left stirring for a further 30 minutes. The solution was then diluted with water (20 mL) and the aqueous layer was extracted with EtOAc (3×20 mL). The combined organic extract was washed with saturated NaCl solution, dried over anhydrous MgSO₄ and filtered. The solvent was removed under reduced pressure and the residue obtained was purified by silica column chromatography (30% EtOAc:n-hexanes) to yield 3β-(tert-butyldimethylsilyloxy)-17β-(2-hydroxyethoxy)-5α-androstane (xx) (312 mg, 62%) as a white solid.

m.p 127-129° C.; ¹H NMR (400 MHz, CDCl₃) δ 3.73-3.63 (m, 2H), 3.62-3.50 (m, 3H), 3.33 (t, J=8.2 Hz, 1H), 2.30-0.80 (m, 21H), 0.88 (s, 9H), 0.80 (s, 3H), 0.75 (s, 3H), 0.59 (m, 1H), 0.04 (s, 6H), OH not observed; ¹³C NMR (101 MHz, CDCl₃) δ 89.7, 72.3, 71.0, 62.3, 54.7, 51.5, 45.2, 43.3, 38.8, 38.2, 37.4, 35.7, 35.5, 32.1, 31.8, 28.8, 28.2, 26.1, 23.5, 21.0, 18.4, 12.5, 11.9, −4.4 (2C); LRMS (ESI+) m/z 473.4 [M+Na]⁺; HRMS (ESI+) m/z 473.3433 [M+Na]⁺, theoretical (C₂₇H₅₀O₃SiNa) m/z 473.3427 [M+Na]⁺; IR 3464, 2936, 2918, 2855 cm⁻¹; specific rotation [α]_{D}²⁵+6.30 (c 1.0, CHCl₃).

### 2-(3β-(Tert-butyldimethylsilyloxy)-5α-androstan-17β-yloxy)-acetic acid

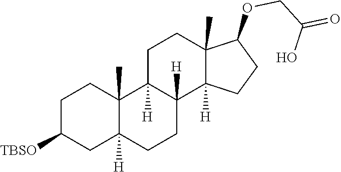

Procedure adapted from Epp and Widlanski, (1999).^{12A} 3β-(Tert-butyldimethylsilyloxy)-17β-(2-hydroxyethoxy)-5α-androstane (50 mg, 0.11 mmol), TEMPO (9.0 mg, 0.55 mmol) and bis(acetoxy)iodobenzene (110 mg, 0.33 mmol) were combined in (v/v) 1:1 CH₂Cl₂/H₂O (4 mL). The reaction was vigorously stirred at room temperature for 4.5 hours. The reaction was quenched with saturated Na₂S₂O₃ solution (1 mL), diluted with water (20 mL), and brought to pH 3 with additions of 5% citric acid solution. The aqueous layer was extracted with CHCl₃ (2×20 mL) and the combined organic extract was washed with saturated NaCl solution, dried over anhydrous MgSO₄ and filtered. The solvent was removed under reduced pressure and the residue obtained was purified by silica column chromatography (2% acetic acid:20% EtOAc:n-hexanes) to yield 2-(3β-(Tert-butyldimethylsilyloxy)-5α-androstan-17β-yloxy)-acetic acid (xx) (39 mg, 76%) as a white oily solid.

¹H NMR (400 MHz, CDCl₃) δ 4.16-4.04 (m, 2H), 3.54 (m, 1H), 3.42 (t, J=8.1 Hz, 1H), 2.06-0.80 (m, 21H), 0.87 (s, 9H), 0.80 (s, 3H), 0.78 (s, 3H), 0.60 (m, 1H), 0.04 (s, 6H) COOH not observed; ¹³C NMR (101 MHz, CDCl₃) δ 173.4, 90.6, 72.3, 67.3, 54.6, 51.2, 45.2, 43.3, 38.8, 38.0, 37.3, 35.7, 35.4, 32.0, 31.7, 28.7, 27.8, 26.1, 23.4, 20.9, 18.4, 12.5, 11.9, −4.4 (2C); LRMS (ESI−) found m/z 463.3 [M−H]⁻; HRMS (ESI+) found m/z 487.3217 [M+Na]⁺, theoretical (C₂₇H₄₈O₄SiNa) m/z 487.3220 [M+Na]⁺; IR 3125, 2487, 1733 cm⁻¹.

### 2-(3β-Hydroxy-5α-androstan-17β-yloxy)-acetate-primaquine amide

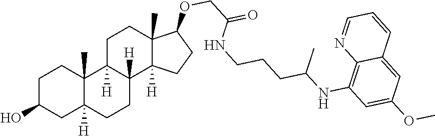

2-(3β-(Tert-butyldimethylsilyloxy)-5α-androstan-17β-yloxy)-acetic acid (29 mg, 0.062 mmol) in CH₂Cl₂ was treated as per general procedure D for 2 days. The reaction mixture was diluted with water (20 mL) and 5% citric acid solution until pH 5. The aqueous layer was further extracted with CH₂Cl₂ (2×20 mL). The combined organic extract was washed with saturated NaHCO₃ solution and saturated NaCl solution, dried over anhydrous MgSO₄ and filtered. The solvent was removed under reduced pressure and the residue obtained was purified by silica column chromatography (50% diethyl ether:CH₂Cl₂) to yield 2-(3β-(tert-butyldimethylsilyloxy)-5α-androstan-17β-yloxy)-acetate-primaquine amide (28 mg, 64%) that was used without further purification in the next step. 2-(3β-(tert-butyldimethylsilyloxy)-5α-androstan-17β-yloxy)-acetate-primaquine amide in MeOH/CH₂Cl₂ (2 mL) was treated as per general procedure E for 1 hour. The reaction was quenched with saturated NaHCO₃ and diluted with water (10 mL). The aqueous layer was extracted with CH₂Cl₂ (2×20 mL). The combined organic extract was washed with saturated NaCl solution, dried over anhydrous MgSO₄ and filtered. The solvent was removed under reduced pressure and the residue obtained was purified by silica column chromatography (8% MeOH:CH₂Cl₂) to yield 2-(3β-Hydroxy-5α-androstan-17β-yloxy)-acetate-primaquine amide (19 mg, 55% over 2 steps) as a green oil.

¹H NMR (400 MHz, MeOD) δ 8.53-8.47 (m, 1H), 8.06-7.98 (m, 1H), 7.40-7.32 (m, 1H), 6.49-6.42 (m, 1H), 6.32-6.27 (m, 1H), 3.89 (s, 2H), 3.87 (s, 3H), 3.71-3.58 (m, 1H), 3.55-3.43 (m, 1H), 3.43-3.32 (m, 1H), 3.28-3.19 (m, 2H), 2.01-0.82 (m, 25H), 1.30-1.28 (m, 3H), 0.79-0.76 (m, 3H), 0.71 (s, 3H), 0.54-0.33 (m, 1H); OH, aniline NH and amide NH not observed; ¹³C NMR (101 MHz, MeOD) δ 172.98/172.92, 161.0, 146.1, 145.3, 136.5, 136.43/136.36, 131.64/131.62, 123.0, 123.0, 98.4, 93.1, 91.29/91.23, 71.8, 70.20/70.04, 55.66/55.58, 52.16/52.06, 48.9, 46.11/46.03, 44.19/44.12, 39.75/39.72, 39.0, 38.88/38.83, 38.15/38.12, 36.59/36.57, 36.54/36.52, 34.85/34.81, 32.65/32.61, 32.1, 29.8, 28.5, 27.0, 24.27/24.23, 21.91/21.86, 20.65/20.60, 12.72/12.69, 12.11/12.08; LRMS (ESI+) found m/z 592.4 [M+H]⁺; HRMS (ESI+) found m/z 592.4111 [M+H]⁺, theoretical (C₃₆H₅₄N₃O₄) m/z 592.4114 [M+H]⁺; IR 3410, 2925, 2852, 1667, 1615, 1594, 1575, 1519 cm⁻¹.

### 2-(3β-(Tert-butyldimethylsilyloxy)-androst-5-en-17β-yloxy)-acetaldehyde

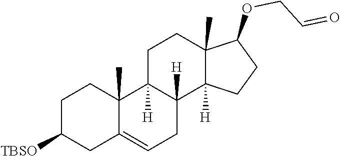

3β-(Tert-butyldimethylsilyloxy)-17β-(2-hydroxyethoxy)-androst-5-ene (40 mg, 0.089 mmol), and DMP (75 mg, 0.17 mmol) in CH₂Cl₂ (2 mL) were stirred at room temperature for 3 hours. The reaction was quenched with saturated Na₂S₂O₃ solution (1 mL) and saturated NaHCO₃ solution (3 mL), diluted with water (10 mL) and extracted with CH₂Cl₂ (2×20 mL). The combined organic extract was washed with saturated NaCl solution, dried over anhydrous MgSO₄ and filtered. The solvent was removed under reduced pressure and the residue obtained was purified by silica column chromatography (10% EtOAc:n-hexanes) to yield 2-(3β-(tert-butyldimethylsilyloxy)-androst-5-en-17β-yloxy)-acetaldehyde (18 mg, 45%) as a white oily solid.

¹H NMR (400 MHz, CDCl₃) δ 9.73 (s, 1H), 5.30 (m, 1H), 4.05 (m, 2H), 3.47 (m, 1H), 3.38 (t, J=8.2 Hz, 1H), 2.29-0.92 (m, 19H), 1.01 (s, 3H), 0.88 (s, 9H), 0.82 (s, 3H), 0.05 (s, 6H); ¹³C NMR (101 MHz, CDCl₃) δ 202.3, 141.8, 120.9, 90.6, 75.8, 72.7, 51.6, 50.4, 43.1, 43.0, 37.9, 37.5, 36.8, 32.2, 31.9, 31.6, 27.9, 26.1, 23.5, 20.8, 19.6, 18.4, 11.7, −4.4 (2C); LRMS (ESI+) found m/z 469.3 [M+Na]⁺; HRMS (ESI+) found m/z 469.3105 [M+Na]⁺, theoretical (C₂₇H₄₆O₃SiNa) m/z 469.3114 [M+Na]⁺; IR 2929, 2856, 1737 cm⁻¹.

### 2-(3β-(Tert-butyldimethylsilyloxy)-5α-androstan-17β-yloxy)-acetaldehyde

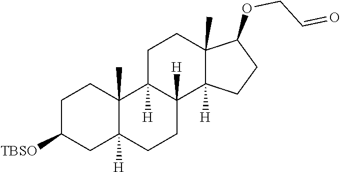

3β-(Tert-butyldimethylsilyloxy)-17 #-(2-hydroxyethoxy)-5α-androstane (31 mg, 0.068 mmol), and DMP (50 mg. 0.12 mmol) in CH₂Cl₂ (2 mL) were stirred at room temperature for 3.5 hours. The reaction was quenched with saturated Na₂S₂O₃ solution (1 mL) and saturated NaHCO₃ solution (3 mL), diluted with water (10 mL) and extracted with CH₂Cl₂ (2×20 mL). The combined organic extract was washed with saturated NaCl solution, dried over anhydrous MgSO₄ and filtered. The solvent was removed under reduced pressure and the residue obtained was purified by silica column chromatography (10% EtOAc:n-hexanes) to yield 2-(3β-(tert-butyldimethylsilyloxy)-5α-androstan-17β-yloxy)-acetaldehyde (18 mg, 58%) as a white oily solid.

¹H NMR (600 MHz, CDCl₃) δ 9.73 (s, 1H), 4.03 (m, 2H), 3.54 (m, 1H), 3.36 (t, J=8.2 Hz, 1H), 2.04-0.83 (m, 21H), 0.88 (s, 9H), 0.80 (s, 3H), 0.79 (s, 3H), 0.63-0.58 (m, 1H), 0.04 (s, 6H); ¹³C NMR (151 MHz, CDCl₃) δ 202.4, 90.6, 75.8, 72.3, 54.7, 51.3, 45.2, 43.3, 38.8, 38.1, 37.4, 35.7, 35.4, 32.1, 31.8, 28.7, 27.9, 26.1, 23.4, 21.0, 18.4, 12.5, 11.9, −4.4 (2C); LRMS (ESI+) found m/z 471.3 [M+Na]⁺; HRMS (ESI+) found m/z 471.3273 [M+Na]⁺, theoretical (C₂₇H₄₈O₃SiNa) m/z 471.3270 [M+Na]⁺; IR 2927, 2856, 1736 cm⁻¹.

### 2-(3β-Hydroxy-5α-androstan-17β-yloxy)-ethyl-primaquine

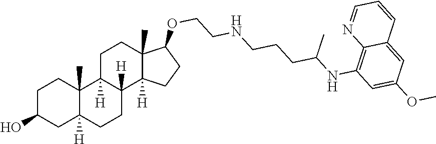

2-(3β-(Tert-butyldimethylsilyloxy)-5α-androstan-17β-yloxy)-acetaldehyde (15 mg, 0.034 mmol) and primaquine bisphosphate (30 mg, 0.067 mmol) were combined in (v/v) 1:1 MeOH/THF (4 mL) and stirred. After 30 minutes, NaCNBH₃ (17 mg, 0.27 mmol) was added and the reaction was stirred at room temperature for 21 hours. The reaction was quenched with saturated NaHCO₃ solution (2 mL), diluted with water (10 mL) and extracted with CH₂Cl₂ (3×15 mL). The combined organic extract was washed with saturated NaCl solution, dried over anhydrous MgSO₄ and filtered. The residue obtained was used without further purification in the next step. The crude 2-(3β-(tert-butyldimethylsilyloxy)-5α-androstan-17β-yloxy)-ethyl-primaquine (17 mg) was treated as per general procedure E in MeOH/CH₂Cl₂ for 1 hour. The reaction was quenched with saturated NaHCO₃ solution, diluted with water (10 mL) and extracted with CH₂Cl₂ (2×20 mL). The combined organic extract was washed with saturated NaCl solution, dried over anhydrous MgSO₄ and filtered. The solvent was removed under reduced pressure and the residue obtained was purified by preparatory HPLC-UV to yield 2-(3β-hydroxy-5α-androstan-17β-yloxy)-ethyl-primaquine (3.1 mg, 16%) as a yellow oil.

¹H NMR (400 MHz, MeOD) δ 8.56-8.50 (m, 1H), 8.12-8.06 (m, 1H), 7.47-7.36 (m, 1H), 6.52 (s, 1H), 6.39 (s, 1H), 3.88 (s, 3H), 3.75-3.50 (m, 4H), 3.26-3.18 (m, 1H), 3.15 (s, 2H), 3.08 (t, J=7.7 Hz, 2H), 1.99-0.84 (m, 25H), 1.34-1.31 (m, 3H), 0.83-0.79 (m, 3H), 0.72-0.66 (m, 3H), 0.64-0.55 (m, 1H), OH, aniline NH and amine NH not observed; LRMS (ESI+) found m/z 578.5 [M+H]⁺; HRMS (ESI+) found m/z 578.4327 [M+H]⁺, theoretical (C₃₀H₅₆N₃O₃) m/z 578.4322 [M+H]⁺.

### 3β-(Tert-butyldimethylsilyloxy)-androst-5-en-17-one

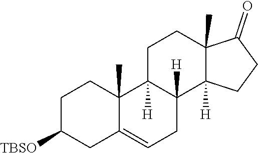

3β-Hydroxyandrost-5-en-17-one (3.50 g, 12.1 mmol) in anhydrous DMF (40 mL) was treated as per general procedure A for 2 h. The reaction mixture was diluted with EtOAc (100 mL) and 5% citric acid solution (100 mL). The aqueous layer was further extracted with EtOAc (2×100 mL). The combined organic extract was washed with saturated NaCl solution, dried over anhydrous MgSO₄ and filtered. The solvent was removed under reduced pressure and the residue obtained was purified by silica column chromatography (20% EtOAc:n-hexanes) to yield 3β-(tert-butyldimethylsilyloxy)-androst-5-en-17-one xx (4.79 g, 98%) as a white solid.

m.p 147-149° C. (lit.^{2A} 149° C.); ¹H NMR (400 MHz, CDCl₃) δ 5.34 (d, J=5.3 Hz, 1H), 3.48 (m, 1H), 2.40-0.95 (m, 19H), 1.02 (s, 3H), 0.89 (s, 9H), 0.88 (s, 3H), 0.06 (s, 6H); ¹³C NMR (101 MHz, CDCl₃) δ 221.3, 141.9, 120.5, 72.6, 52.0, 50.5, 47.7, 42.9, 37.5, 36.9, 36.0, 32.2, 31.7, 31.6, 31.0, 26.1, 22.0, 20.5, 19.6, 18.4, 13.7, −4.4 (2C); LRMS (GCMS) found m/z 345 [(M−C₄H₉)+H]⁺; HRMS (ESI+): found m/z 425.2834 [M+Na]⁺, theoretical m/z 425.2834 [M+Na]⁺; IR 2946, 2857, 1746 cm⁻¹; specific rotation [α]_{D}²⁵−38.63 (c 1.0, CHCl₃).

### 3β-(Triethylsilyloxy)-androst-5-en-17-one

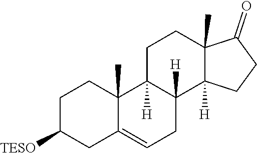

Procedure adapted from Yamauchi et al., (2005).^{2A} Triethylsilyl chloride (0.790 g, 7.80 mmol) was added to a solution of 3β-Hydroxyandrost-5-ene-17-one (1.50 g, 5.20 mmol) and imidazole (366 mg, 8.32 mmol) in anhydrous DMF (40 mL). The solution was stirred at room temperature for 24 hours. The reaction mixture was diluted with water (100 mL) and extracted with EtOAc (2×20 mL). The combined organic extract was washed with saturated NaCl solution, dried over anhydrous MgSO₄ and filtered. The solvent was removed under reduced pressure and the residue obtained was purified by silica column chromatography (20% EtOAc:n-hexanes) to yield 3β-(triethylsilyloxy)-androst-5-en-17-one (1.76 g, 84%) as a white solid.

m.p 55-58° C.; ¹H NMR (400 MHz, CDCl₃) δ 5.33 (m, 1H), 3.47 (m, 1H), 2.48-1.05 (m, 19H), 1.01 (s, 3H), 0.95 (t, J=7.9 Hz, 9H), 0.87 (s, 3H), 0.58 (q, J=7.9 Hz, 6H); ¹³C NMR (101 MHz, CDCl₃) MHz, CDCl₃) δ (101 MHz, CDCl₃) δ 221.2, 141.9, 120.6, 72.3, 51.9, 50.5, 47.7, 43.0, 37.5, 36.9, 36.0, 32.2, 31.7, 31.6, 31.0, 22.0, 20.5, 19.6, 13.7, 7.0, 5.0; LRMS (ESI+) found m/z 425.2 [M+Na]⁺; HRMS (ESI+) found m/z 425.2864 [M+Na]⁺, theoretical (C₂₅H₄₂O₂SiNa) m/z 425.2852 [M+Na]⁺; IR 2935, 2875, 1738 cm⁻¹; specific rotation [α]_{D}²⁵+5.20 (c 1.0, CHCl₃).

### 3β-(Tert-butyldimethylsilyloxy)-androst-5-en-17β-ol

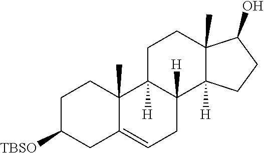

3β-(Tert-butyldimethylsilyloxy)-androst-5-en-17-one xx (2.85 g, 7.08 mmol) in ice bath cooled THF (20 mL) and MeOH (20 mL) was treated as per general procedure B for 2.5 hours. The reaction mixture was diluted with CH₂Cl₂ (40 mL) and water (20 mL), then treated with 2M HCl solution (5 mL). The aqueous layer was further extracted with CH₂Cl₂ (2×50 mL). The combined organic extract was washed with saturated NaCl solution, dried over anhydrous MgSO₄ and filtered. The solvent was removed under reduced pressure and the residue obtained was purified by silica column chromatography (20% EtOAc:n-hexanes) to yield 3β-(tert-butyldimethylsilyloxy)-androst-5-en-17β-ol xx (2.78 g, 97%) as a white solid.

m.p 170-172° C. (lit.^{14A} 171-172° C.); ¹H NMR (400 MHz, CDCl₃) δ 5.31 (d, J=4.6 Hz, 1H), 3.64 (t, J=8.5 Hz, 1H), 3.52-3.42 (m, 1H), 2.30-0.93 (m, 19H), 1.01 (s, 3H), 0.89 (s, 9H), 0.75 (s, 3H), 0.05 (s, 6H), OH not observed; ¹³C NMR (101 MHz, CDCl₃) δ 141.8, 121.0, 82.1, 72.7, 51.5, 50.5, 43.0, 42.9, 37.6, 36.8, 36.8, 32.2, 32.1, 31.7, 30.7, 26.1, 23.6, 20.8, 19.6, 18.4, 11.1, −4.4 (2C); LRMS (GCMS) found m/z 347 [(M−C₄H₉)+H]⁺; HRMS (ESI+) found m/z 427.2992 [M+Na]⁺, theoretical m/z 427.3002 [M+Na]⁺; IR 3305, 2929, 2853 cm⁻¹;

specific rotation [α]_{D}²⁵−38.63 (c 1.0, CHCl₃).

### 3β-(Triethylsilyloxy)-androst-5-en-17β-ol

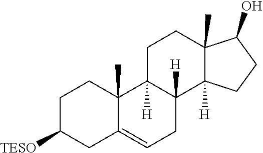

3β-(Triethylsilyloxy)-androst-5-en-17-one (1.06 g, 2.63 mmol) in ice bath cooled THF (20 mL) and MeOH (20 mL) was treated as per general procedure B for 4.5 hours. The reaction mixture was diluted with water (30 mL) and extracted with EtOAc (2×30 mL). The combined organic extract was washed with saturated NaCl solution, dried over anhydrous MgSO₄ and filtered. The solvent was removed under reduced pressure and the residue obtained was purified by silica column chromatography (20% EtOAc:n-hexanes) to yield 30-(triethylsilyloxy)-androst-5-ene-17β-ol (1.01 g, 95%) as a white solid.

m.p 138-140° C.; ¹H NMR (400 MHz, CDCl₃) δ 5.32 (m, 1H), 3.64 (t, J=8.5 Hz, 1H), 3.47 (m, 1H), 2.32-1.04 (m, 19H), 1.01 (s, 3H), 0.96 (t, J=7.9 Hz, 9H), 0.76 (s, 3H), 0.59 (q, J=7.9 Hz, 6H), OH not observed; ¹³C NMR (101 MHz, CDCl₃) δ 141.8, 121.0, 82.1, 72.4, 51.5, 50.5, 43.0, 42.9, 37.6, 36.8, 36.8, 32.3, 32.1, 31.7, 30.7, 23.6, 20.8, 19.6, 11.1, 7.0, 5.0; LRMS (ESI+) found m/z 427.3 [M+Na]⁺; HRMS (ESI+) found m/z 427.3009 [M+Na]⁺, theoretical (C₂H₄₄O₂SiNa) m/z 427.3008 [M+Na]⁺; IR 3285, 2981 cm⁻¹; specific rotation [α]_{D}²⁵−1.10 (c 1.0, CHCl₃).

### 3β-(Tert-butyldimethylsilyloxy)-androst-5-en-17β-yl hemisuccinate

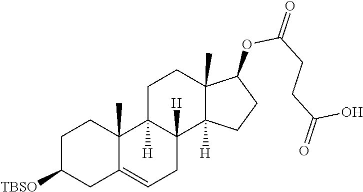

3β-(Tert-butyldimethylsilyloxy)-androst-5-en-17β-ol (0.640 g, 1.58 mmol) in anhydrous toluene (20 mL) was treated as per general procedure C for 24 hours. The reaction mixture was diluted with CH₂Cl₂ (40 mL), water (20 mL), then treated with 2M HCl solution until pH=2. The aqueous layer was extracted with CH₂Cl₂ (2×40 mL). The combined organic extract was washed with saturated NaCl solution, dried over anhydrous MgSO₄ and filtered. The solvent was removed under reduced pressure and the residue obtained was purified by silica column chromatography (5% AcOH:20% EtOAc:n-hexanes) to yield 3β-(tert-butyldimethylsilyloxy)-androst-5-en-17β-yl hemisuccinate (xx) (0.763 g, 95%) as a white solid.

m.p 164-168° C.; ¹H NMR (400 MHz, CDCl₃) δ 5.31 (d, J=4.3 Hz, 1H), 4.62 (t, J=8.4 Hz, 1H), 3.47 (m, 1H), 2.72-2.59 (m, 4H), 2.30-1.05 (m, 19H) 1.00 (s, 3H), 0.88 (s, 9H), 0.79 (s, 3H), 0.05 (s, 6H), COOH not observed; ¹³C NMR (101 MHz, CDCl₃) δ 177.9, 172.2, 141.8, 120.9, 83.4, 72.7, 51.2, 50.3, 42.9, 42.6, 37.5, 36.9, 36.8, 32.2, 31.9, 31.6, 29.3, 29.2, 27.6, 26.1, 23.7, 20.7, 19.6, 18.4, 12.1, −4.4 (2C); LRMS (ESI−) found m/z 503.2 [M−H]⁻; HRMS (ESI−) found m/z 503.3192 [M−H]⁻ theoretical (C₂₉H₄₇O₅Si) m/z 503.3193 [M−H]⁻; IR 3000, 2964, 1735, 1705 cm⁻¹; Specific rotation [α]_{D}²⁵−10.30 (c 1.0, CHCl₃).

### 3β-(Triethylsilyloxy)-androst-5-en-17β-yl hemisuccinate

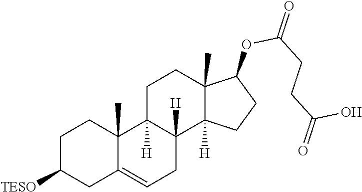

3β-(Triethylsilyloxy)-androst-5-en-17β-ol (700 mg, 1.73 mmol) in anhydrous toluene (10 mL) was treated as per general procedure C for 24 hours. The reaction mixture was diluted with water (40 mL) and extracted with CH₂Cl₂ (3×50 mL). The combined organic extract was washed with saturated NaCl solution, dried over anhydrous MgSO₄ and filtered. The solvent was removed under reduced pressure and the residue obtained was purified by silica column chromatography (8% MeOH:CH₂Cl₂) to yield 3β-(triethylsilyloxy)-androst-5-en-17β-yl hemisuccinate (350 mg, 40%) as a white solid.

m.p 133-135° C.; ¹H NMR (400 MHz, CDCl₃) δ 5.31 (m, 1H), 4.63 (t, J=8.4 Hz, 1H), 3.47 (m, 1H), 2.69-2.61 (m, 4H), 2.32-0.87 (m, 19H), 1.00 (s, 3H), 0.96 (t, J=7.9 Hz, 9H), 0.79 (s, 3H), 0.59 (q, J=7.9 Hz, 6H) COOH not observed; ¹³C NMR (101 MHz, CDCl₃) δ 177.3, 172.2, 141.8, 120.9, 83.4, 72.4, 51.2, 50.3, 43.0, 42.7, 37.6, 36.9, 36.8, 32.2, 31.9, 31.6, 29.3, 29.1, 27.6, 23.7, 20.7, 19.6, 12.1, 7.0, 5.0; LRMS (ESI−) found m/z 503.3 [M−H]⁻; HRMS (ESI−) found m/z 503.3190 [M−H]⁻, theoretical (C₂₉H₄₇O₅Si) m/z 503.3193 [M−H]⁻.

### 3α-(Tert-butyldimethylsilyloxy)-androst-5-en-17β-yl succinate-primaquine amide

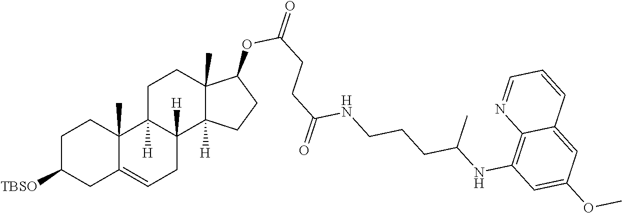

3β-(Tert-butyldimethylsilyloxy)-androst-5-en-17β-yl hemisuccinate (0.175 g, 0.347 mmol) in CH₂Cl₂ (20 mL) was treated as per general procedure D for 2 days. The reaction mixture was diluted with water (20 mL) and 5% citric acid solution until pH 5. The aqueous layer was further extracted with CH₂Cl₂ (3×30 mL). The combined organic extract was washed with saturated NaHCO₃ solution and saturated NaCl solution, dried over anhydrous MgSO₄ and filtered. The solvent was removed under reduced pressure and the residue obtained was purified by silica column chromatography (3% MeOH:CH₂Cl₂) to yield 3β-(tert-butyldimethylsilyloxy)-androst-5-en-17β-yl succinate-primaquine amide (180 mg, 76%) as a green oil.

¹¹H NMR (400 MHz, CD₂Cl₂) δ 8.50 (d, J=4.1 Hz, 1H), 7.94 (d, J=8.2 Hz, 1H), 7.31 (dd, J=8.2, 4.2 Hz, 1H), 6.35 (d, J=2.2 Hz, 1H), 6.27 (d, J=2.2 Hz, 1H), 6.03 (d, J=8.4 Hz, 1H), 5.69 (s, 1H), 5.30 (s, 1H), 4.56 (t, J=8.4 Hz, 1H), 3.87 (s, 3H), 3.63 (s, 1H), 3.47 (tt, J=10.4, 4.7 Hz, 1H), 3.29-3.17 (m, 2H), 2.58 (t, J=6.7 Hz, 2H), 2.38 (t, J=6.8 Hz, 2H), 2.27-0.88 (m, 23H), 1.28 (d, J=6.4 Hz, 3H), 0.99 (s, 3H), 0.88 (s, 9H), 0.77 (s, 3H), 0.05 (s, 6H); ¹³C NMR (101 MHz, CD₂Cl₂) δ 173.2, 171.5, 159.9, 145.4, 144.7, 142.0, 135.7, 135.1, 130.3, 122.3, 121.1, 97.0, 92.0, 83.3, 72.9, 55.5, 51.4, 50.6, 48.2, 43.2, 42.8, 39.8, 37.7, 37.2, 37.0, 34.4, 32.5, 32.1, 31.9, 31.5, 30.2, 27.9, 26.8, 26.1, 23.9, 21.0, 20.7, 19.6, 18.4, 12.1, −4.5 (2C); LRMS (ESI+) found m/z 746.5 [M+H]⁺; HRMS (ESI+) found m/z 746.4929 [M+H]⁺, theoretical (C₄₄H₆₈N₃O₅) m/z 746.4928 [M+H]⁺; IR 3379, 3314, 2929, 2854, 1731, 1648, 1615, 1595, 1576, 1519 cm⁻¹.

### 3β-Hydroxyandrost-5-en-17β-yl succinate-primaquine amide (C-17-prim; BC5B)

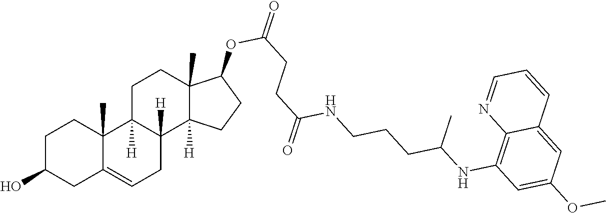

3β-(Tert-butyldimethylsilyloxy)-androst-5-en-17β-yl succinate-primaquine amide (150 mg, 0.201 mmol) in CH₂Cl₂/MeOH (10 mL) was treated as per general procedure E for 1 hour. The reaction was quenched with saturated NaHCO₃ and diluted with water (30 mL). The aqueous layer was extracted with CH₂Cl₂ (3×30 mL). The combined organic extract was washed with saturated NaCl solution, dried over anhydrous MgSO₄ and filtered. The solvent was removed under reduced pressure and the residue obtained was purified by silica column chromatography (5% MeOH:CH₂Cl₂) to yield 3β-hydroxyandrost-5-en-17β-yl succinate-primaquine amide (91 mg, 54% over 2 steps) as a green oily solid.

¹H NMR (400 MHz, MeOD) δ 8.55-8.48 (m, 1H), 8.06 (d, J=8.2 Hz, 1H), 8.01-7.94 (m, 1H), 7.42-7.35 (m, 1H), 6.51-6.44 (m, 1H), 6.35 (s, 1H), 5.32 (d, J=4.6 Hz, 1H), 4.56-4.48 (m, 1H), 3.87 (s, 3H), 3.70-3.59 (m, 1H), 3.44-3.35 (m, 1H), 3.26-3.15 (m, 2H), 2.59 (t, J=6.0 Hz, 2H), 2.45 (t, J=6.7 Hz, 2H), 2.27-0.77 (m, 23H), 1.28 (m, 3H), 0.98/0.96 (m, 3H), 0.77/0.76 (m, 3H), OH and aniline NH not observed; ¹³C NMR (101 MHz, MeOD) δ 174.18/174.17, 174.1, 161.0, 146.2, 145.3, 142.2, 136.5, 136.3, 131.6, 122.9, 122.1, 98.35/98.33, 93.0, 84.3, 72.3, 55.7, 52.16/52.11, 51.49/51.46, 48.6, 43.6/43.59, 43.0, 40.4, 38.4, 37.94/37.93, 37.7, 35.00/34.99, 32.9, 32.4, 32.3, 31.6, 30.69/30.68, 28.4, 27.22/27.20, 24.4, 21.6, 20.7, 19.9, 12.4; LRMS (ESI+) found m/z 632.4 [M+H]⁺; HRMS found m/z 632.4061 [M+H]⁺, theoretical (C₃₈H₅₄N₃O₅) m/z 632.4063 [M+H]⁺; IR 3316, 2932, 1728, 1651, 1615, 1575, 1519 cm⁻¹.

### 3β-(Tert-butyldimethylsilyloxy)-androst-5-en-17β-yl succinate-propan-2-yl diester

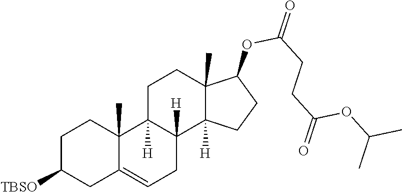

3β-(Tert-butyldimethylsilyloxy)-androst-5-en-17β-ol (0.200 g, 0.495 mmol) in CH₂Cl₂ (3 mL) was treated as per general procedure F for 21 hours. The reaction was diluted with EtOAc (20 mL), water (20 mL) and 5% citric acid solution (5 mL). The aqueous layer was further extracted with EtOAc (2×20 mL). The combined organic extract was washed with saturated NaCl solution, dried over anhydrous Na₂SO₄ and filtered. The solvent was removed under reduced pressure and the residue obtained was purified by silica column chromatography (20% EtOAc:n-hexanes) to yield 3β-(tert-butyldimethylsilyloxy)-androst-5-en-17β-yl succinate-propan-2-yl diester (131 mg, 49%) as a colourless oil.

¹H NMR (400 MHz, CDCl₃) δ; ¹H NMR (400 MHz, CDCl₃) δ 5.31 (d, J=4.9 Hz, 1H), 5.01 (hept, J=6.3 Hz, 1H), 4.61 (t, J=8.4 Hz, 1H), 3.47 (tt, J=10.7, 4.7 Hz, 1H), 2.65-2.54 (m, J=4.4 Hz, 4H), 2.30-0.91 (m, 19H), 1.23 (d, J=6.3 Hz, 6H), 1.00 (s, 3H), 0.88 (s, 9H), 0.80 (s, 3H), 0.05 (s, 6H); ¹³C NMR (101 MHz, CDCl₃) δ; 172.4, 171.9, 141.8, 120.9, 83.2, 72.7, 68.2, 51.2, 50.3, 42.9, 42.6, 37.5, 36.9, 36.8, 32.2, 31.9, 31.6, 29.8, 29.6, 27.6, 26.1, 23.7, 22.0 (2C), 20.7, 19.6, 18.4, 12.1, −4.4 (2C); LRMS (ESI+) found m/z 547.4 [M+H]⁺; HRMS (ESI+) found m/z 569.3638 [M+Na]⁺, theoretical (C₃₂H₅₄O₅SiNa) m/z 569.3638 [M+Na]⁺; IR 2938, 1729 cm⁻¹; specific rotation [α]_{D}²⁵−44.00 (c 1.0, CHCl₃).

### 3β-Hydroxyandrost-5-en-17β-yl succinate-propan-2-yl diester (C-17-Link)

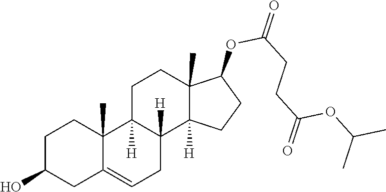

3β-(Tert-butyldimethylsilyloxy)-androst-5-en-17β-yl succinate-propan-2-yl diester (80 mg, 0.15 mmol) in CH₂Cl₂ (5 mL) was treated as per general procedure E for 1.5 hours. The reaction was quenched with saturated NaHCO₃ and diluted with water (20 mL) and CH₂Cl₂ (15 mL). The aqueous layer was extracted with CH₂Cl₂ (2×20 mL). The combined organic extract was washed with saturated NaCl solution, dried over anhydrous Na₂SO₄ and filtered. The solvent was removed under reduced pressure and the residue obtained was purified by silica column chromatography (20% EtOAc:n-hexanes) to yield 3β-hydroxyandrost-5-en-17β-yl succinate-propan-2-yl diester (53 mg, 84%) as a white solid.

m.p 105-107° C.; ¹H NMR (400 MHz, CDCl₃) δ 5.34 (d, J=5.4 Hz, 1H), 5.01 (hept, J=6.3 Hz, 1H), 4.66-4.57 (m, 1H), 3.52 (tt, J=11.1, 4.5 Hz, 1H), 2.63-2.55 (m, 4H), 2.32-0.91 (m, 21H), 1.23 (d, J=6.3 Hz, 6H), 1.01 (s, 3H), 0.80 (s, 3H), OH not observed; ¹³C NMR (101 MHz, CDCl₃) δ 172.4, 171.9, 141.0, 121.4, 83.2, 71.8, 68.2, 51.2, 50.2, 42.6, 42.4, 37.4, 36.9, 36.7, 31.8, 31.7, 31.6, 29.7, 29.6, 27.6, 23.7, 21.9 (2C), 20.7, 19.6, 12.1; LRMS (ESI+) found m/z 433.3 [M+H]⁺; HRMS (ESI+) found m/z 455.2773 [M+Na]⁺, theoretical (C₂₆H₄₀O₅Na) m/z 455.2773 [M+Na]⁺; IR 3443, 2933, 1730 cm⁻¹; specific rotation [α]_{D}²⁵−38.80 (c 1.0, CHCl₃).

### 3β-(Tert-butyldimethylsilyloxy)-5α-androstan-17-one

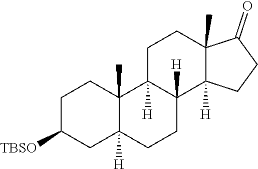

3β-Hydroxy-5α-androstan-17-one (1.00 g, 3.4 mmol) in anhydrous DMF (15 mL) was treated as per general procedure A for 1 hours. The reaction mixture was diluted with EtOAc (40 mL) and water (40 mL). The aqueous layer was further extracted with EtOAc (2×50 mL). The combined organic extract was washed with saturated NaCl solution (20 mL), dried over anhydrous MgSO₄ and filtered. The solvent was removed under reduced pressure and the residue obtained was purified by silica column chromatography (20% EtOAc:n-hexanes) to yield 3β-(tert-butyldimethylsilyloxy)-5α-androstan-17-one xx (1.28 g, 92%) as a white solid.

m.p 160-162° C. (lit.^{15A} 161-163° C.); ¹H NMR (400 MHz, CDCl₃) δ 3.61-3.49 (m, 1H), 2.48-2.37 (m, 1H), 2.12-1.99 (m, 1H), 1.98-1.87 (m, 1H), 1.80-0.92 (m, 18H), 0.88 (s, 9H), 0.85 (s, 3H), 0.82 (s, 3H), 0.72-0.61 (m, 1H), 0.05 (s, 6H); ¹³C NMR (101 MHz, CDCl₃) δ 221.6, 72.1, 54.7, 51.6, 48.0, 45.2, 38.8, 37.3, 36.0, 35.8, 35.2, 32.0, 31.7, 31.1, 28.6, 26.1, 21.9, 20.6, 18.4, 14.0, 12.5, −4.4 (2C); LRMS (ESI+) found m/z 427.3 [M+Na]⁺; IR 2928, 1746 cm⁻¹; specific rotation [α]_{D}²⁵+62.70 (c 1.0, CHCl₃).

### 3β-(Tert-butyldimethylsilyloxy)-5α-androstan-17β-ol

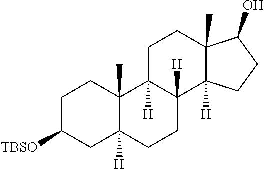

3β-(Tert-butyldimethylsilyloxy)-5α-androstan-17-one xx (1.20 g, 2.96 mmol) in ice bath cooled THF (15 mL) and MeOH (10 mL) was treated as per general procedure xx for 2.5 h. The reaction mixture was diluted with CH₂Cl₂ (20 mL) and water (10 mL), then treated with 2M HCl solution until pH=7. The aqueous layer was further extracted with CH₂Cl₂ (2×50 mL). The combined organic extract was washed with saturated NaCl solution (30 mL), dried over anhydrous MgSO₄ and filtered. The solvent was removed under reduced pressure and the residue obtained was purified by silica column chromatography (20% EtOAc:n-hexanes) to yield 3β-(tert-butyldimethylsilyloxy)-5α-androstan-17β-ol xx (1.04 g, 86%) as a white solid.

m.p 179-181° C.; ¹H NMR (400 MHz, CDCl₃) δ 3.62 (t, J=8.5 Hz, 1H), 3.58-3.48 (m, 1H), 2.11-1.97 (m, 1H), 1.79-0.83 (m, 20H), 0.88 (s, 9H), 0.80 (s, 3H), 0.72 (s, 3H), 0.63-0.56 (m, 1H), 0.04 (s, 6H), OH not observed; ¹³C NMR (101 MHz, CDCl₃) δ 82.1, 72.3, 54.7, 51.2, 45.3, 43.2, 38.8, 37.4, 37.0, 35.8, 35.7, 32.1, 31.9, 30.7, 28.8, 26.1, 23.6, 21.0, 18.4, 12.6, 11.3, −4.4 (2C); LRMS (ESI+) found m/z 429.3 [M+Na]⁺; IR 3254, 2926, 2851 cm⁻¹; specific rotation [α]_{D}²⁵+5.30 (c 1.0, CHCl₃).

### 3β-(Tert-butyldimethylsilyloxy)-5α-androstan-17β-yl hemisuccinate

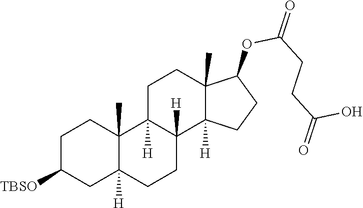

3β-(Tert-butyldimethylsilyloxy)-5α-androstan-17β-ol (0.520 g. 1.28 mmol) in anhydrous toluene was treated as per general procedure C for 24 hours. The reaction mixture was diluted with CH₂Cl₂ (20 mL) and water (23 mL), then treated with 2M HCl solution until pH=2. The aqueous layer was further extracted with CH₂Cl₂ (2×20 mL). The combined organic extract was washed with saturated NaCl solution, dried over MgSO₄ and filtered. The solvent was removed under reduced pressure and the residue obtained was purified by silica column chromatography (8% MeOH:CH₂Cl₂) to yield 3β-(tert-butyldimethylsilyloxy)-5α-androstan-17β-yl-hemisuccinate xx (0.431 g, 67%) as a white solid.

m.p 145-147° C.; ¹H NMR (400 MHz, CDCl₃) δ 4.61 (t, J=8.3 Hz, 1H), 3.60-3.48 (m, 1H), 2.75-2.55 (m, 4H), 2.20-2.06 (m, 1H), 1.79-0.91 (m, 20H), 0.88 (s, 9H), 0.80 (s, 3H), 0.76 (s, 3H), 0.67-0.57 (m, 1H), 0.04 (s, 6H), COOH not observed; ¹³C NMR (101 MHz, CDCl₃) δ 177.5, 172.2, 83.5, 72.3, 54.6, 50.9, 45.2, 42.9, 38.8, 37.3, 37.1, 35.7, 35.4, 32.1, 31.8, 29.3, 29.1, 28.8, 27.6, 26.1, 23.7, 20.8, 18.4, 12.5, 12.3, −4.4 (2C); LRMS (ESI−) found m/z 505.3 [M−H]⁻; HRMS (ESI−) found m/z 505.3349 [M−H]⁻, theoretical (C₂₉H₄₉O₅Si) m/z 505.3349 [M−H]⁻; IR 2928, 2855, 1716 cm⁻¹; specific rotation [α]_{D}²⁵+3.60 (c 1.0, CHCl₃).

### 3β-(Tet-butyldimethylsilyloxy)-5α-androstan-17β-yl succinate-primaquine amide

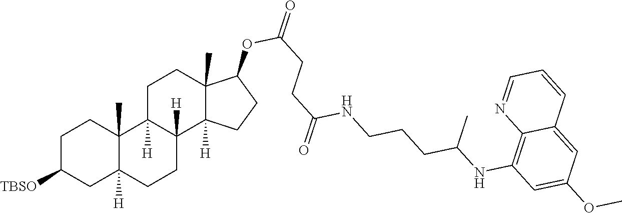

3β-(tert-butyldimethylsilyloxy)-5α-androstan-17f-yl-hemisuccinate (50 mg, 0.98 mmol) in CH₂Cl₂ was treated as per general procedure D for 2 days. The reaction mixture was diluted with water (20 mL), 5% citric acid solution (5 mL) and CH₂Cl₂ (10 mL). The aqueous layer was further extracted with CH₂Cl₂ (2×20 mL). The combined organic extract was washed with saturated NaHCO₃ solution and saturated NaCl solution, dried over anhydrous MgSO₄ and filtered. The solvent was removed under reduced pressure and the residue obtained was purified by silica column chromatography (5% MeOH:CH₂Cl₂) to yield 3β-(tert-butyldimethylsilyloxy)-5α-androstan-17β-yl succinate-primaquine amide (43 mg, 58%) as a green oil.

¹H NMR (400 MHz, MeOD) δ 8.48 (d, J=4.1 Hz, 1H), 8.01 (d, J=8.2 Hz, 1H), 7.97-7.91 (m, 1H), 7.35 (dd, J=8.2, 4.1 Hz, 1H), 6.43 (s, 1H), 6.30 (s, 1H), 4.51 (t, J=7.3 Hz, 1H), 3.86 (s, 3H), 3.71-3.50 (m, 2H), 3.26-3.12 (m, 2H), 2.64-2.52 (m, 2H), 2.44 (t, J=6.7 Hz, 2H), 2.12-1.96 (m, 1H), 1.77-0.79 (m, 23H), 1.28 (d, J=6.1 Hz, 3H), 0.89 (s, 9H), 0.76 (d, J=4.3 Hz, 3H), 0.73 (d, J=3.2 Hz, 3H), 0.57-0.45 (m, 1H), 0.06 (s, 6H), OH and aniline NH not observed; ¹³C NMR (101 MHz, MeOD) δ 174.2, 174.2, 161.0, 146.2, 145.3, 136.5, 136.4, 131.6, 122.9, 98.4/98.3, 93.0, 84.4, 73.5, 55.7, 55.63/55.60, 51.9/51.8, 49.0, 46.1, 43.88/43.86, 40.4, 39.8, 38.2, 38.14/38.12, 36.6, 36.53/36.52, 35.02/34.99, 33.0, 32.7, 31.6, 30.7, 29.8, 28.4, 27.22/27.18, 26.4, 24.4, 21.8, 20.7, 19.0, 12.7, 12.6, −4.4 (2C); LRMS (ESI+) found m/z 748.5 [M+H]⁺; HRMS (ESI+) found m/z 748.5085 [M+H]⁺, theoretical (C₄₄H₇₀N₃O₅Si) m/z 748.5085 [M+H]⁺; IR 3384, 2927, 2854, 2475, 1732, 1637, 1616, 1577, 1520 cm⁻¹.

### 3β-Hydroxy-5α-androstan-17α-yl succinate-primaquine amide

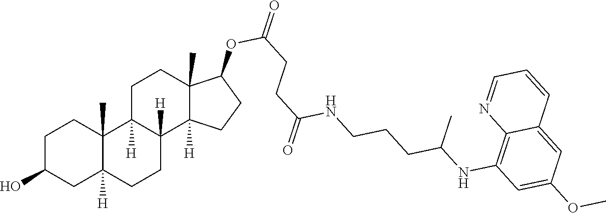

3β-(Tert-butyldimethylsilyloxy)-5α-androstan-17β-yl succinate-primaquine amide (24 mg, 0.031 mmol) in MeOH/CH₂Cl₂ (5 mL) was treated as per general procedure E for 30 minutes. The reaction was quenched with saturated NaHCO₃ and diluted with water (10 mL) and CH₂Cl₂ (20 mL). The aqueous layer was further extracted with CH₂Cl₂ (2×20 mL). The combined organic extract was washed with saturated NaCl solution, dried over anhydrous MgSO₄ and filtered. The solvent was removed under reduced pressure and the residue obtained was purified by silica column chromatography (5% MeOH:CH₂Cl₂) to yield 3β-hydroxy-5α-androstan-17β-yl succinate-primaquine amide (16 mg, 81%) as a green oil.

¹H NMR (400 MHz, MeOD) δ 8.48 (s, 1H), 8.02 (d, J=8.2 Hz, 1H), 7.35 (dd, J=8.1, 4.2 Hz, 1H), 6.44 (s, 1H), 6.30 (s, 1H), 4.52 (t, J=8.2 Hz, 1H), 3.87 (s, 3H), 3.67-3.59 (m, 1H), 3.53-3.45 (m, 1H), 3.25-3.15 (m, 2H), 2.62-2.54 (m, 2H), 2.48-2.41 (m, 2H), 2.05 (s, 1H), 1.74-0.72 (m, 21H), 1.28 (d, J=6.1 Hz, 6H), 0.78/0.77 (s, 3H), 0.74 (s, 3H), 0.59-0.47 (m, 1H), OH, aniline NH and amide NH not observed; ¹³C NMR (101 MHz, MeOD) δ 174.2, 174.2, 161.0, 146.2, 145.3, 136.5, 136.4, 131.6, 122.9, 98.40/98.36, 93.1, 84.4, 71.8, 55.7 (2C, HSQC), 51.90/51.86, 49.13 (HSQC), 46.1, 43.9, 40.4, 38.9, 38.2, 36.6, 36.5, 35.01/34.98, 32.7, 32.1, 31.6, 30.8, 30.7, 29.8, 28.4, 27.22/27.19, 24.4, 21.8, 20.7, 12.7, 12.6; LRMS (ESI+) found m/z 634.5 [M+H]⁺; HRMS (ESI+) found m/z 634.4219 [M+H]⁺, theoretical (C₃₈H₅₆N₃O₅) m/z 634.4220 [M+H]⁺; IR 3377, 2925, 2853, 1729, 1646, 1615 1576, 1519 cm⁻¹.

### 3β-(Tert-butyldimethylsilyloxy)-5α-androstan-17β-yl succinate-propan-2-yl diester

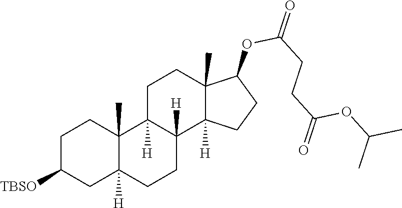

3β-(Tert-butyldimethylsilyloxy)-5α-androstan-17β-ol (50 mg, 0.12 mmol) in CH₂Cl₂ (3 mL) was treated as per general procedure F for 21 hours. The reaction was diluted with EtOAc (20 mL), water (20 mL) and 5% citric acid solution (5 mL). The aqueous layer was further extracted with EtOAc (2×20 mL). The combined organic extract was washed with saturated NaCl solution solution, dried over anhydrous MgSO₄ and filtered. The solvent was removed under reduced pressure and the residue obtained was purified by silica column chromatography (20% EtOAc:n-hexanes) to yield 3β-(tert-butyldimethylsilyloxy)-5α-androstan-17β-yl succinate-propan-2-yl diester (31 mg, 46%) as a colourless oil.

¹H NMR δ 5.09-4.93 (m, 1H), 4.60 (t, J=8.3 Hz, 1H), 3.61-3.47 (m, 1H), 2.65-2.52 (m, 4H), 2.22-2.06 (m, 1H), 1.72-0.84 (m, 20H), 1.23 (d, J=6.1 Hz, 6H), 0.88 (s, 9H), 0.80 (s, 3H), 0.77 (s, 3H), 0.68-0.55 (m, 1H), 0.04 (s, 6H); ¹³C NMR (101 MHz, CDCl₃) δ 172.4, 172.0, 83.3, 72.3, 68.2, 54.6, 50.9, 45.2, 42.9, 38.8, 37.3, 37.1, 35.7, 35.4, 32.1, 31.8, 29.8, 29.6, 28.8, 27.7, 26.1, 23.7, 22.0 (2C), 20.8, 18.4, 12.5, 12.3, −4.4 (2C); LRMS (ESI+) found 571.4 m/z [M+Na]⁺; HRMS (ESI+) found m/z 571.3795 [M+Na]⁺, theoretical (C₃₂H₅₆N₅O₅SiNa) m/z 571.3795 [M+Na]⁺; IR 2926, 2850, 1731 cm⁻; specific rotation [α]_{D}²⁵+0.70 (c 1.0, CHCl₃).

### 3β-Hydroxy-5α-androstan-17β-yl succinate-propan-2-yl diester

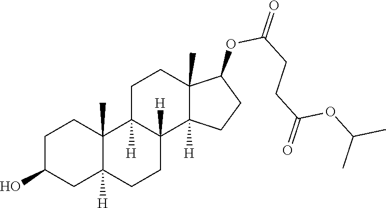

3β-(Tert-butyldimethylsilyloxy)-5α-androstan-17β-yl succinate-propan-2-yl diester (20 mg, 0.036 mmol) in MeOH/CH₂Cl₂ (4 mL) was treated as per general procedure E for 20 minutes. The reaction was quenched with saturated NaHCO₃ and diluted with water (20 mL). The aqueous layer was extracted with CH₂Cl₂ (2×20 mL). The combined organic extract was washed with saturated NaCl solution, dried over anhydrous MgSO₄ and filtered. The solvent was removed under reduced pressure and the residue obtained was purified by silica column chromatography (30% EtOAc:n-hexanes) to yield 3β-hydroxy-5α-androstan-17β-yl succinate-propan-2-yl diester (14 mg, 89%) as a colourless oil.

¹H NMR (400 MHz, CDCl₃) δ 5.08-4.93 (m, 1H), 4.59 (t, J=8.4 Hz, 1H), 3.64-3.52 (m, 1H), 2.64-2.53 (m, 4H), 2.20-2.05 (m, 1H), 1.82-0.88 (m, 20H), 1.22 (d, J=6.3 Hz, 6H), 0.81 (s, 3H), 0.77 (s, 3H), 0.68-0.58 (m, 1H), OH not observed; ¹³C NMR (101 MHz, CDCl₃) δ 172.4, 171.9, 83.3, 71.4, 68.2, 54.4, 50.9, 45.0, 42.9, 38.3, 37.1, 37.1, 35.7, 35.4, 31.7, 31.6, 29.8, 29.6, 28.7, 27.6, 23.7, 22.0 (2C), 20.8, 12.5, 12.3; LRMS (ESI+) found 435.3 m/z [M+H]⁺; IR 3445, 2928, 1732 cm⁻¹.

### 3α-(Tert-butyldimethylsilyloxy)-5α-androstan-17-one

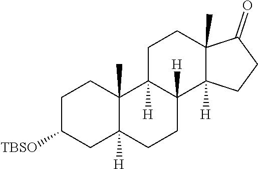

3α-Hydroxy-5α-androstan-17-one (0.300 g, 0.769 mmol) in anhydrous DMF (5 mL) was treated as per general procedure A for 27 h. The reaction mixture was diluted with EtOAc (20 mL) and 5% citric acid solution (30 mL). The aqueous layer was further extracted with EtOAc (2×30 mL). The combined organic extract was washed with saturated NaCl solution, dried over anhydrous MgSO₄ and filtered. The solvent was removed under reduced pressure and the residue obtained was purified by silica column chromatography (20% EtOAc:n-hexanes) to yield 3α-(tert-butyldimethylsilyloxy)-5α-androstan-17-one xx (0.376 g, 89%) as a white oily solid.

¹H NMR (400 MHz, CDCl₃) δ 3.98-3.94 (m, 1H), 2.47-2.37 (m, 1H), 2.11-1.99 (m, 1H), 1.97-1.88 (m, 1H), 1.79-0.91 (m, 19H), 0.88 (s, 9H), 0.85 (s, 3H), 0.77 (s, 3H), 0.01 (s, 6H); ¹³C NMR (101 MHz, CDCl₃) δ 221.7, 66.9, 54.6, 51.6, 48.0, 39.2, 36.9, 36.3, 36.0, 35.2, 32.5, 31.7, 31.1, 29.8, 28.5, 26.0, 21.9, 20.2, 18.3, 14.0, 11.5, −4.7 (2C); LRMS (ESI+) found m/z 831.6 [M2+Na]⁺; HRMS (ESI+): found m/z 427.3008 [M+Na]⁺, theoretical (C₂₅H₄₄O₂SiNa) m/z 427.3008 [M+Na]⁺; IR 2925, 2855, 1741 cm⁻¹; specific rotation [α]_{D}²⁵+67.30 (c 1.0, CHCl₃).

### 3α-(Tert-butyldimethylsilyloxy)-5α-androstan-17β-ol

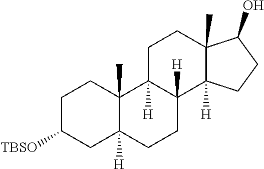

3α-(Tert-butyldimethylsilyloxy)-5α-androstan-17-one xx (0.315 g, 0.780 mmol) in ice bath cooled THF (4 mL) and MeOH (4 mL) was treated as per general procedure B for 2.5 h. The reaction mixture was diluted with CH₂Cl₂ (20 mL) and water (20 mL), then treated with 2M HCl solution until pH=2. The aqueous layer was further extracted with CH₂Cl₂ (2×15 mL). The combined organic extract was washed with saturated NaCl solution, dried over anhydrous MgSO₄ and filtered. The solvent was removed under reduced pressure and the residue obtained was purified by silica column chromatography (20% EtOAc:n-hexanes) to yield 3α-(tert-butyldimethylsilyloxy)-Sa-androstan-17β-ol xx (0.283 g, 90%) as a white solid.

m.p 139-141° C.; ¹H NMR (400 MHz, CDCl₃) δ 3.95 (s, 1H), 3.61 (m, 1H), 2.11-1.98 (m, 1H), 1.72-0.71 (m, 21H), 0.88 (s, 9H), 0.76 (s, 3H), 0.73 (s, 3H), 0.01 (s, 6H), OH not observed; ¹³C NMR (101 MHz, CDCl₃) δ 82.2, 67.0, 54.7, 51.2, 43.2, 39.3, 37.0, 36.9, 36.2, 35.7, 32.6, 31.9, 30.7, 29.9, 28.7, 26.0, 23.5, 20.5, 18.3, 11.6, 11.3, −4.7 (2C); LRMS (ESI+) found m/z 835.7 [2M+Na]⁺; HRMS (ESI+) found m/z 429.3165 [M+Na]⁺, theoretical (C₂₅H₄₆O₂SiNa) m/z 429.3165 [M+Na]⁺; IR 3411, 2927.2853 cm⁻¹; specific rotation [α]_{D}²⁵+6.10 (c 1.0, CHCl₃).

### 3α-(Tert-butyldimethylsilyloxy)-5α-androstan-17β-yl hemisuccinate

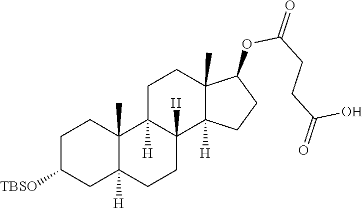

3α-(Tert-butyldimethylsilyloxy)-5α-androstan-17β-ol xx (0.180 g, 0.443 mmol) in anhydrous toluene (8 mL) was treated as per general procedure C for 24 hours. The reaction mixture was diluted with CH₂Cl₂ (20 mL) and 2M HCl solution (30 mL). The aqueous layer was further extracted with CH₂Cl₂ (2×15 mL). The combined organic extract was washed with saturated NaCl solution, dried over anhydrous MgSO₄ and filtered. The solvent was removed under reduced pressure and the residue obtained was purified by silica column chromatography (5% AcOH:20% EtOAc:n-hexanes) to yield 3α-(tert-butyldimethylsilyloxy)-5α-androstan-17β-yl hemisuccinate xx (0.195 g, 87%) as a white solid.

m.p 125-128° C.; ¹H NMR (400 MHz, CDCl₃) δ 4.60 (t, J=8.4 Hz, 1H), 3.99-3.92 (m, 1H), 2.74-2.56 (m, 4H), 2.21-2.09 (m, 1H), 1.71-0.92 (m, 21H), 0.88 (s, 9H), 0.77 (s, 3H), 0.75 (s, 3H), 0.01 (s, 6H), COOH not observed; ¹³C NMR (101 MHz, CDCl₃) δ 178.2, 172.2, 83.7, 67.0, 54.4, 50.9, 42.9, 39.2, 37.1, 36.9, 36.2, 35.5, 32.6, 31.8, 29.9, 29.3, 29.3, 28.7, 27.7, 26.0, 23.7, 20.4, 18.3, 12.3, 11.6, −4.7 (2C); LRMS (ESI−) found m/z 505.3 [M−H]⁻; HRMS (ESI−) found m/z 505.3349 [M−H]⁻, theoretical (C₂₉H₄₉O₅Si) m/z 505.3349 [M−H]⁻; IR 3027, 2926, 2854, 1736, 1713 cm⁻¹; specific rotation [α]_{D}²⁵+14.00 (c 1.0, CHCl₃).

### 3α-Hydroxy-5α-androstan-17R-yl succinate-primaquine amide

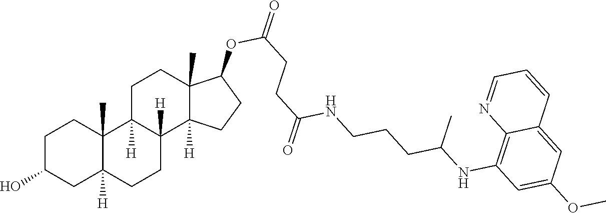

3α-(Tert-butyldimethylsilyloxy)-5α-androstan-17β-yl hemisuccinate (50 mg, 0.099 mmol) in CH₂Cl₂ (4 mL) was treated as per general procedure D for 4 days. The reaction mixture was diluted with CH₂Cl₂ (20 mL) and 5% citric acid solution (20 mL). The aqueous layer was further extracted with CH₂Cl₃ (2×20 mL). The combined organic extract was washed with saturated NaHCO₃ solution and saturated NaCl solution, dried over anhydrous MgSO₄ and filtered. The solvent was removed under reduced pressure and the residue obtained was purified by silica column chromatography (50% EtOAc:n-hexanes) to yield slightly impure 3α-(tert-butyldimethylsilyloxy)-Sa-androstan-17β-yl succinate-primaquine amide (70 mg) which was used without further purification. 3α-(Tert-butyldimethylsilyloxy)-5α-androstan-17β-yl succinate-primaquine amide (70 mg, 0.094 mmol) in MeOH/CH₂Cl₂ was treated as per general procedure E for 1.5 hours. The reaction was quenched with saturated NaHCO₃ and diluted with water (20 mL) and CH₂Cl₂ (20 mL). The aqueous layer was further extracted with CH₂Cl₂ (2×20 mL). The combined organic extract was washed with saturated NaCl solution, dried over anhydrous MgSO₄ and filtered. The solvent was removed under reduced pressure and the residue obtained was purified by silica column chromatography (5% MeOH:CH₂Cl₂) to yield 3α-hydroxy-5α-androstan-17β-yl succinate-primaquine amide (35 mg, 56% over 2 steps) as a green oil.

¹H NMR (400 MHz, MeOD) δ 8.48 (d, J=4.0 Hz, 1H), 8.01 (d, J=8.2 Hz, 1H), 7.94 (m, 1H), 7.38-7.31 (m, 1H), 6.43 (s, 1H), 6.30 (s, 1H), 4.50 (t, J=8.3 Hz, 1H), 3.96-3.91 (m, 1H), 3.86 (s, 3H), 3.67-3.57 (m, 1H), 3.25-3.11 (m, 2H), 2.57 (t, J=6.4 Hz, 2H), 2.44 (t, J=6.6 Hz, 2H), 2.11-1.98 (m, 1H), 1.77-0.57 (m, 25H), 1.27 (d, J=6.2 Hz, 3H), 0.77-0.74 (m, 3H), 0.73 (s, 3H), OH and aniline NH not overserved; ¹³C NMR (101 MHz, MeOD) δ 174.2, 174.2, 161.0, 146.2, 145.31/145.28, 136.5, 136.4, 131.6, 122.95/122.92, 98.4, 93.0, 84.4, 67.1, 55.7, 55.7, 52.0, 48.8 (HSQC), 43.8, 40.4, 40.2, 38.1, 37.1, 36.7, 36.5, 35.0, 33.4, 32.7, 31.6, 30.7, 29.6, 29.6, 28.4, 27.2, 24.4, 21.3, 20.7, 12.6, 11.7; LRMS (ESI+) found m/z 656.4 [M+Na]⁺; HRMS (ESI+) found m/z 656.4039 [M+Na]⁺, theoretical (C₃₈H₅₅N₃O₅Na) m/z 656.4039 [M+Na]⁺.

### 3α-(Tert-butyldimethylsilyloxy)-5α-androstan-17β-yl succinate-propan-2-yl diester

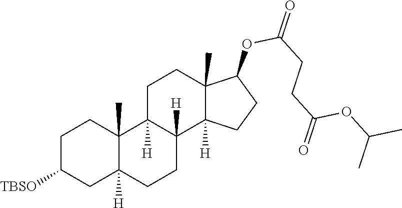

3α-(Tert-butyldimethylsilyloxy)-5α-androstan-17β-ol (49 mg, 0.12 mmol) in CH₂Cl₂ (3 mL) was treated as per general procedure F overnight. The reaction was diluted with CH₂Cl₂ (20 mL) and 5% citric acid solution (20 mL). The aqueous layer was further extracted with CH₂Cl₂ (2×15 mL). The combined organic extract was washed with saturated NaCl solution, dried over anhydrous MgSO₄ and filtered. The solvent was removed under reduced pressure and the residue obtained was purified by silica column chromatography (20% EtOAc:n-hexanes) to yield 3α-(tert-butyldimethylsilyloxy)-5α-androstan-17β-yl succinate-propan-2-yl diester (61 mg, 92%) as a colourless oil.

¹H NMR (400 MHz, CDCl₃) δ 5.00 (hept, J=6.3 Hz, 1H), 4.58 (t, J=8.4 Hz, 1H), 4.00-3.89 (m, 1H), 2.64-2.53 (m, 4H), 2.20-2.07 (m, 1H), 1.72-0.91 (m, 21H), 1.22 (d, J=6.3 Hz, 6H), 0.88 (s, 9H), 0.77 (s, 3H), 0.75 (s, 3H), 0.00 (s, 6H); ¹³C NMR (101 MHz, CDCl₃) δ 172.4, 171.9, 83.4, 68.1, 67.0, 54.4, 50.9, 42.9, 39.2, 37.1, 36.9, 36.2, 35.4, 32.5, km, /31.8, 29.9, 29.8, 29.6, 28.6, 27.7, 26.0, 23.7, 21.9 (2C), 20.4, 18.2, 12.3, 11.5, −4.7 (2C); LRMS (ESI+) found 571.4 m/z [M+Na]⁺; HRMS (ESI+) found m/z 571.3795 [M+Na]⁺, theoretical (C₃₂H₅₆O₅SiNa) m/z 571.3795 [M+Na]⁺; IR 2927, 2854, 1734 cm⁻¹; specific rotation [α]_{D}²⁵+8.70 (c 1.0, CHCl₃).

### 3α-Hydroxy-5α-androstan-17α-yl succinate-propan-2-yl diester

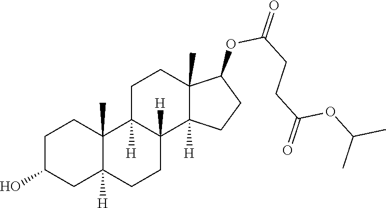

3α-(Tert-butyldimethylsilyloxy)-5α-androstan-17β-yl succinate-propan-2-yl diester (38 mg, 0.069 mmol) in MeOH/CH₂Cl₂ (4 mL) was treated as per general procedure E for 3 hours. The reaction was quenched with saturated NaHCO₃ and diluted with water (20 mL) and CH₂Cl₂ (20 mL). The aqueous layer was further extracted with CH₂Cl₂ (2×15 mL). The combined organic extract was washed with saturated NaCl solution, dried over anhydrous Na₂SO₄ and filtered. The solvent was removed under reduced pressure and the residue obtained was purified by silica column chromatography (20% EtOAc:n-hexanes) to yield 3α-hydroxy-5α-androstan-17β-yl succinate-propan-2-yl diester (20 mg, 67%) as a colourless oil.

¹H NMR (400 MHz, CDCl₃) δ 5.01 (hept, J=6.2 Hz, 1H), 4.60 (t, J=8.4 Hz, 1H), 4.08-4.00 (m, 1H), 2.64-2.55 (m, 4H), 2.20-2.07 (m, 1H), 1.74-0.83 (m, 21H), 1.23 (d, J=6.2 Hz, 6H), 0.79-0.76 (m, 6H), OH not observed; ¹³C NMR (101 MHz, CDCl₃) δ 172.4, 171.9, 83.3, 68.1, 66.6, 54.4, 50.9, 42.9, 39.3, 37.1, 36.3, 36.0, 35.4, 32.3, 31.7, 29.8, 29.6, 29.1, 28.5, 27.6, 23.6, 21.9 (2C), 20.4, 12.3, 11.3; LRMS (ESI+) found 457.3 m/Z [M+Na]⁺; HRMS (ESI+) found m/z 457.2930 [M+Na]⁺, theoretical (C₂₆H₄₂O₅Na) m/z 457.2930 [M+Na]⁺; IR 3286, 2930, 1732 cm⁻; specific rotation [α]_{D}²⁵+2.00 (c 1.0, CHCl₃).

### 3α-(Tert-butyldimethylsilyloxy)-5β-androstan-17-one

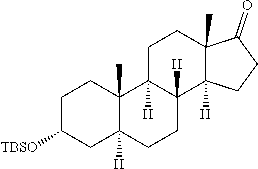

3α-Hydroxy-5β-androstan-17-one (0.210 g, 0.724 mmol) in anhydrous DMF (4 mL) was treated as per general procedure A for 3 h. The reaction mixture was diluted with EtOAc (10 mL) and 5% citric acid solution (30 mL). The aqueous layer was further extracted with EtOAc (2×30 mL). The combined organic extract was washed with saturated NaCl solution, dried over anhydrous Na₂SO₄ and filtered. The solvent was removed under reduced pressure and the residue obtained was purified by silica column chromatography (20% EtOAc:n-hexanes) to yield 3α-(tert-butyldimethylsilyloxy)-5β-androstan-17-one xx (0.284 g, 97%) as a white solid.

m.p 95-97° C.; ¹H NMR (400 MHz, CDCl₃) δ 3.65-3.53 (m, 1H), 2.49-2.36 (m, 1H), 2.14-2.00 (m, 1H), 1.96-0.91 (m, 20H), 0.93 (s, 3H), 0.89 (s, 9H), 0.84 (s, 3H), 0.05 (s, 6H); ¹³C NMR (101 MHz, CDCl₃) δ 221.6, 72.7, 51.6, 48.0, 42.4, 40.7, 37.0, 36.1, 35.7, 35.6, 34.9, 31.9, 31.1, 27.2, 26.1, 25.5, 23.5, 22.0, 20.2, 18.5, 13.9, −4.5 (2C); LRMS (ESI+) found m/z 427.3 [M+Na]⁺; HRMS (ESI+) found m/z 427.3008 [M+Na]⁺, theoretical (C₂₅H₄₄O₂SiNa) m/z 427.3008 [M+Na]⁺; IR 2928, 2857, 1741 cm⁻¹; specific rotation [α]_{D}²⁵+56.30 (c 1.0, CHCl₃).

### 3α-Tert-butydimethylsilyoxy-5-androstan-17β-ol

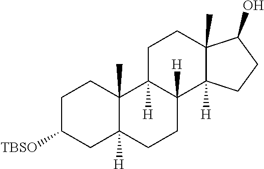

3α-(Tert-butyldimethylsilyloxy)-5β-androstan-17-one xx (0.235 g, 0.582 mmol) in ice bath cooled THF (3 mL) and MeOH (3 mL) was treated as per general procedure B for 2 h. The reaction mixture was diluted with CH₂Cl₂ (20 mL) and water (20 mL), then treated with 2M HCl solution until pH=2. The aqueous layer was further extracted with CH₂Cl₂ (2×20 mL). The combined organic extract was washed with saturated NaCl solution, dried over anhydrous Na₂SO₄ and filtered. The solvent was removed under reduced pressure and the residue obtained was purified by silica column chromatography (20% EtOAc:n-hexanes) to yield 3α-(tert-butyldimethylsilyloxy)-5β-androstan-17β-ol xx (0.197 g, 83%) as a white solid.

m.p 111-113° C.; ¹H NMR (400 MHz, CDCl₃) δ 3.62 (t, J=8.5 Hz, 1H), 3.60-3.53 (m, 1H), 2.12-1.98 (m, 1H), 1.86-1.71 (m, 4H), 1.61-0.94 (m, 17H), 0.91 (s, 3H), 0.89 (s, 9H), 0.71 (s, 3H), 0.05 (s, 6H), OH not observed; ¹³C NMR (101 MHz, CDCl₃) δ 82.1, 72.9, 51.1, 43.2, 42.4, 40.6, 37.0 (2C, HSQC), 36.1, 35.8, 34.8, 31.1, 30.7, 27.3, 26.1, 26.1, 23.6, 23.5, 20.5, 18.5, 11.2, −4.5 (2C); LRMS (ESI+) found 835.7 m/z [M2+Na]⁺; HRMS (ESI+) found m/z 429.3165 [M+Na]⁺, theoretical (C₂₅H₄₆O₂SiNa) m/z 429.3165 [M+Na]⁺; IR 3363, 2927, 2854 cm⁻¹; specific rotation [α]_{D}²⁵+2.30 (c 1.0, CHCl₃).

### 3α-(Tert-butyldimethylsilyloxy)-5β-androstan-17β-yl hemisuccinate

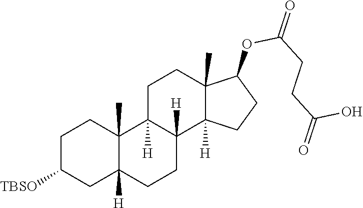

3α-(Tert-butyldimethylsilyloxy)-5β-androstan-17β-ol xx (0.148 g, 0.365 mmol) in anhydrous toluene (6 mL) was treated as per general procedure C for 24.5 hours. The reaction mixture was diluted with CH₂Cl₂ (20 mL), water (20 mL) and brought to pH 2 by addition of 2M HCl solution. The aqueous layer was further extracted with CH₂Cl₂ (2×20 mL). The combined organic extract was washed with saturated NaCl solution, dried over anhydrous Na₂SO₄ and filtered. The solvent was removed under reduced pressure and the residue obtained was purified by silica column chromatography (5% AcOH:20% EtOAc:n-hexanes) to yield 3α-(tert-butyldimethylsilyloxy)-5β-androstan-17β-yl hemisuccinate xx (92.5 mg. 50%) as a white oily solid.

¹H NMR (400 MHz, CDCl₃) δ 12.16-9.88 (s, 1H), 4.59 (t, J=8.4 Hz, 1H), 3.62-3.52 (m, 1H), 2.70-2.58 (m, 4H), 2.21-2.10 (m, 1H), 1.87-0.93 (m, 21H), 0.90 (s, 3H), 0.88 (s, 9H), 0.75 (s, 3H), 0.05 (s, 6H); ¹³C NMR (101 MHz, CDCl₃) δ 178.3, 172.1, 83.6, 72.9, 50.8, 42.9, 42.4, 40.4, 37.2, 37.0, 35.8, 35.8, 34.8, 31.1, 29.3, 29.2, 27.7, 27.3, 26.12, 26.11, 23.7, 23.5, 20.4, 18.5, 12.2, −4.5 (2C); LRMS (ESI−) found m/z 503.3 [M−H]⁻; HRMS (ESI+) found m/z 529.3325 [M+Na]⁺, theoretical (C₂₉H₅₀O₅SiNa) m/z 529.3325 [M+Na]⁺; IR 3084, 2928, 2856, 1736, 1714 cm⁻¹; specific rotation [α]_{D}²⁵+12.80 (c 1.0, CHCl₃).

### 3α-(Tert-butyldimethylsilyloxy)-5β-androstan-17β-yl succinate-primaquine amide

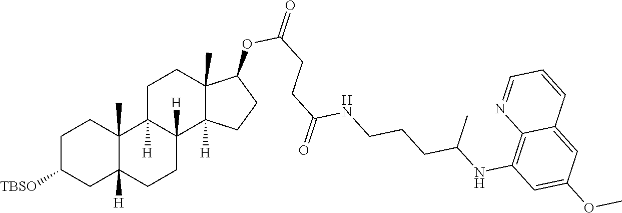

3α-(Tert-butyldimethylsilyloxy)-5β-androstan-17β-yl hemisuccinate (30 mg, 0.059 mmol) in CH₂Cl₂ (4 mL) was treated as per general procedure D for 23 hours. The reaction mixture was diluted with CH₂Cl₂ (15 mL), water (10 mL) and 5% citric acid solution until pH 4. The aqueous layer was further extracted with CH₂Cl₂ (2×15 mL). The combined organic extract was washed with saturated NaHCO₃ solution and saturated NaCl solution, dried over anhydrous Na₂SO₄ and filtered. The solvent was removed under reduced pressure and the residue obtained was purified by silica column chromatography (3% MeOH:CH₂Cl₂) to yield 3α-(tert-butyldimethylsilyloxy)-50-androstan-17β-yl succinate-primaquine amide (38 mg, 85%) as a green oil.

¹H NMR (400 MHz, MeOD) δ 8.49 (s, 1H), 8.09-8.01 (m, 1H), 7.41-7.31 (m, 1H), 6.50-6.43 (m, 1H), 6.31 (s, 1H), 4.58-4.45 (m, 1H), 3.87 (s, 3H), 3.64 (s, 2H), 3.26-3.15 (m, 2H), 2.58 (t, J=6.8 Hz, 2H), 2.44 (t, J=6.6 Hz, 2H), 2.14-2.00 (m, 1H), 1.94-0.86 (m, 25H), 1.30-1.27 (m, 3H), 0.90 (s, 9H), 0.73 (s, 3H), 0.07 (s, 6H), amide NH and aniline NH not observed; ¹³C NMR (101 MHz, MeOD) δ 174.2, 174.2, 161.0, 146.2, 145.3, 136.5, 136.4, 131.6, 123.0, 98.4, 93.1, 84.4, 74.0, 55.7, 51.9, 48.8 (HSQC), 44.0, 43.5, 41.8, 40.4, 38.3, 38.1, 36.9, 36.6, 35.7, 34.9, 32.0, 31.6, 30.7, 28.5, 28.2, 27.2, 27.1, 26.4, 24.4, 23.9, 21.4, 20.7, 19.0, 12.5, −4.4 (2C).

### 3α-Hydroxy-5β-androstan-17β-yl succinate-primaquine amide

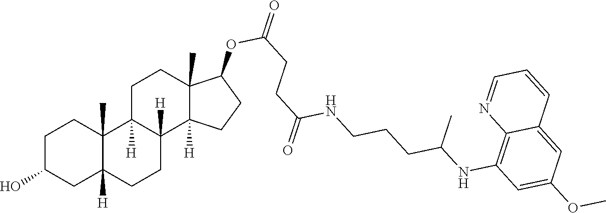

3α-(Tert-butyldimethylsilyloxy)-5β-androstan-17β-yl succinate-primaquine amide (28 mg, 0.037 mmol) in MeOH/CH₂Cl₂ was treated as per general procedure E for 2 hours. The reaction was quenched with saturated NaHCO₃ and diluted with water (20 mL) and CH₂Cl₂ (20 mL). The aqueous layer was further extracted with CH₂Cl₂ (2×20 mL). The combined organic extract was washed with saturated NaCl solution, dried over anhydrous Na₂SO₄ and filtered. The solvent was removed under reduced pressure and the residue obtained was purified by silica column chromatography (5% MeOH:CH₂Cl₂) to yield 3α-hydroxy-5β-androstan-17β-yl succinate-primaquine amide (23 mg, 97%) as a green oil.

¹H NMR (400 MHz, CD₂Cl₂) δ 8.50 (d, J=2.3 Hz, 1H), 7.94 (d, J=8.2 Hz, 1H), 7.35-7.28 (m, 1H), 6.36 (s, 1H), 6.27 (s, 1H), 6.03 (d, J=6.7 Hz, 1H), 5.72 (s, 1H), 5.34-5.32 (m, 1H), 4.55 (t, J=8.4 Hz, 1H), 3.87 (s, 3H), 3.66-3.52 (m, 2H), 3.30-3.17 (m, 2H), 2.58 (t, J=6.8 Hz, 2H), 2.38 (t, J=6.6 Hz, 2H), 2.16-2.05 (m, 1H), 1.89-0.88 (m, 25H), 1.28 (d, J=6.5 Hz, 3H), 0.91 (s, 3H), 0.74 (s, 3H); ³C NMR (101 MHz, CD₂Cl₂) δ 173.2, 171.5, 159.9, 145.4, 144.7, 135.7, 135.1, 130.3, 122.3, 97.0, 92.0, 83.4, 72.0, 55.6, 51.1, 48.2, 43.2, 42.6, 40.9, 39.8, 37.5, 36.9, 36.0, 35.8, 35.0, 34.3, 31.5, 31.0, 30.2, 28.0, 27.5, 26.8, 26.4, 23.9, 23.5, 20.7, 20.7, 12.2; LRMS (ESI+) found m/z 634.4 [M+H]⁺; HRMS (ESI+) found m/z 634.4220 [M+H]⁺, theoretical (C₃₈H₅₆N₃O₅) m/z 634.4220 [M+H]⁺.

### 3α-(Tert-butyldimethylsilyloxy)-5β-androstan-17β-yl succinate-propan-2-yl diester

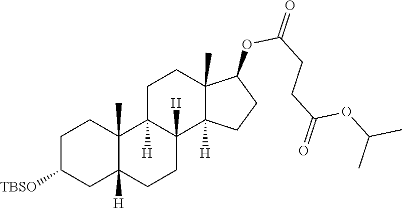

3α-(Tert-butyldimethylsilyloxy)-5β-androstan-17β-ol (30 mg, 0.074 mmol) in CH₂Cl₂ (3 mL) was treated as per general procedure F for 24 hours. The reaction was diluted with CH₂Cl₂ (20 mL) and 5% citric acid solution (20 mL). The aqueous layer was further extracted with CH₂Cl₂ (2×20 mL). The combined organic extract was washed with saturated NaCl solution solution, dried over anhydrous Na₂SO₄ and filtered. The solvent was removed under reduced pressure and the residue obtained was purified by silica column chromatography (10% EtOAc:n-hexanes) to yield 3α-(tert-butyldimethylsilyloxy)-5β-androstan-17β-yl succinate-propan-2-yl diester (24 mg, 62%) as a colourless oil.

¹H NMR (400 MHz, CDCl₃) δ 5.01 (hept, J=6.2 Hz, 1H), 4.58 (t, J=7.7 Hz, 1H), 3.66-3.50 (m, 1H), 2.66-2.56 (m, 4H), 2.19-2.11 (m, 1H), 1.87-0.92 (m, 21H), 1.23 (d, J=6.2 Hz, 6H), 0.90 (s, 3H), 0.88 (s, 9H), 0.76 (s, 3H), 0.05 (s, 6H); ³C NMR (101 MHz, CDCl₃) δ 172.4, 171.9, 83.4, 72.9, 68.1, 50.8, 42.9, 42.4, 40.5, 37.2, 37.1, 35.8, 35.8, 34.8, 31.1, 29.8, 29.6, 27.8, 27.3, 26.1, 26.1, 23.7, 23.5, 22.0 (2C), 20.4, 18.5, 12.2, −4.4, −4.5; LRMS (ESI+) found 1119.8 m/z [2M+Na]⁺; HRMS (ESI+) found m/z 571.3795 [M+Na]⁺, theoretical (C₃₂H₅₆O₅SiNa) m/z 571.3795 [M+Na]⁺; IR 2929, 2857, 1735 cm⁻¹.

### 3α-Hydroxy-5β-androstan-17α-yl succinate-propan-2-yl diester

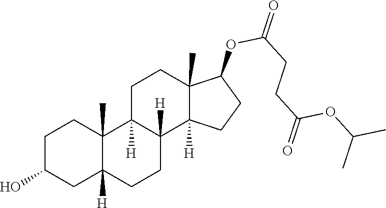

3α-(Tert-butyldimethylsilyloxy)-5β-androstan-17β-yl succinate-propan-2-yl diester (13 mg, 0.024 mmol) in MeOH/CH₂Cl₂ (2 mL) was treated as per general procedure E for 30 minutes. The reaction was quenched with saturated NaHCO₃ and diluted with water (15 mL) and CH₂Cl₂ (15 mL). The aqueous layer was further extracted with CH₂Cl₂ (2×15 mL). The combined organic extract was washed with saturated NaCl solution, dried over anhydrous Na₂SO₄ and filtered. The solvent was removed under reduced pressure and the residue obtained was purified by silica column chromatography (20% EtOAc:n-hexanes) to yield 3α-hydroxy-5α-androstan-17β-yl succinate-propan-2-yl diester (8.3 mg, 80%) as a colourless oil.

¹H NMR (400 MHz, CDCl₃) δ 5.01 (hept, J=6.2 Hz, 1H), 4.60 (t, J=8.4 Hz, 1H), 3.73-3.53 (m, 1H), 2.67-2.50 (m, 4H), 2.26-2.07 (m, 1H), 1.90-0.95 (m, 21H), 1.23 (d, J=6.2 Hz, 6H), 0.93 (s, 3H), 0.77 (s, 3H), OH not observed; ¹³C NMR (101 MHz, CDCl₃) δ 172.4, 172.0, 83.3, 71.9, 68.2, 50.9, 43.0, 42.2, 40.7, 37.2, 36.6, 35.8, 35.5, 34.8, 30.7, 29.8, 29.6, 27.7, 27.2, 26.2, 23.7, 23.5, 22.0 (2C), 20.4, 12.3; LRMS (ESI+) found m/z 891.6 [M2+Na]⁺.

### Androst-5-en-17-one-3β-yl hemisuccinate

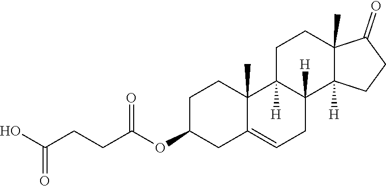

Procedure adapted from Marek et al., (2016).^{16A} Succinic anhydride (0.17 g, 1.7 mmol) was added to a stirring solution of 3β-hydroxyandrost-5-en-17-one (0.200 g, 0.69 mmol) and DMAP (70 mg, 0.57 mmol) in pyridine (3 mL). The solution was heated to 60° C. and stirred for 19 h. The reaction mixture was diluted with CH₂Cl₂ (20 mL) and 5% citric acid solution (20 mL). The aqueous layer was further extracted with CH₂Cl, (2×15 mL). The combined organic extract was washed with 2M HCl solution (2×20 mL), then with saturated NaCl solution, dried over anhydrous MgSO₄ and filtered. The solvent was removed under reduced pressure and the residue obtained was purified by silica column chromatography (5% AcOH:50% EtOAc:n-hexanes) to yield androst-5-en-17-one-3β-yl hemisuccinate xx (0.173 g, 64%) as a white solid.

m.p 237-239° C. (lit.^{17A} 232-234° C.); ¹H NMR (400 MHz, CDCl₃) δ 5.40 (d, J=4.5 Hz, 1H), 4.70-4.56 (m, 1H), 2.70-2.57 (m, 4H), 2.51-2.41 (m, 1H), 2.40-2.26 (m, 2H), 2.16-2.02 (m, 2H), 1.98-0.99 (m, 14H), 1.04 (s, 3H), 0.88 (s, 3H), COOH not observed; ¹³C NMR (101 MHz, CDCl₃) δ 221.4, 177.8, 171.7, 139.9, 122.1, 74.4, 51.8, 50.3, 47.7, 38.1, 37.0, 36.9, 36.0, 31.6, 31.5, 30.9, 29.4, 29.1, 27.8, 22.0, 20.5, 19.5 13.7; LRMS (ESI−) found m/z 387.2 [M−H]⁻; HRMS (ESI−) found m/z 387.2172 [M−H]⁻, theoretical (C₂₃H₃₁O₅) m/z 387.2171 [M−H]⁻.

### Androst-5-en-17-one-3β-yl succinate-primaquine amide

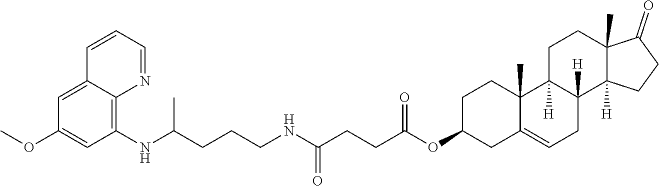

Androst-5-en-17-one-3β-yl hemisuccinate (32 mg, 0.085 mmol) in CH₂Cl₂ (4 mL) was treated as per general procedure D for 2 days. The reaction mixture was diluted with CH₂Cl₂ (20 mL) and 5% citric acid solution (30 mL). The aqueous layer was further extracted with CH₂Cl₂ (2×15 mL). The combined organic extract was washed with saturated NaHCO₃ solution and saturated NaCl solution, dried over anhydrous MgSO₄ and filtered. The solvent was removed under reduced pressure and the residue obtained was purified by silica column chromatography (5% MeOH:CH₂Cl₂) to yield androst-5-en-17-one-3β-yl succinate-primaquine amide (23 mg, 44%).

¹H NMR (400 MHz, CDCl₃) δ 8.52 (d, J=4.2 Hz, 1H), 7.92 (d, J=8.2 Hz, 1H), 7.30 (dd, J=8.2, 4.2 Hz, 1H), 6.33 (d, J=2.3 Hz, 1H), 6.27 (d, J=2.2 Hz, 1H), 5.89-5.82 (m, 1H), 5.37 (d, J=4.7 Hz, 1H), 4.65-4.54 (m, 1H), 3.88 (s, 3H), 3.65-3.57 (m, 1H), 3.34-3.20 (m, 2H), 2.62 (t, J=6.8 Hz, 2H), 2.49-2.39 (m, 3H), 2.06-0.88 (m, 22H), 1.28 (d, J=6.4 Hz, 3H), 1.01 (s, 3H), 0.87 (s, 3H), aniline NH not observed; LRMS (ESI+) found m/z 652.4 [M+Na]⁺; HRMS (ESI+) found m/z 652.3727 [M+Na]⁺, theoretical (C₃₈H₅₁N₃O₅Na) m/z 652.3726 [M+Na]⁺; IR 3382, 3327, 2939, 1576, 1735, 1650, 1615, 1576, 1519 cm⁻¹.

### Androst-5-en-17-one-3β-yl succinate-propan-2-yl diester

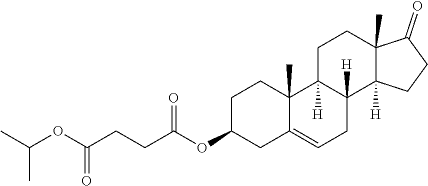

3β-Hydroxyandrost-5-en-17-one (100 mg, 3.5 mmol) in CH₂Cl₂ (3 mL) was treated as per general procedure E for 20 hours. The reaction was diluted with CH₂Cl₂ (20 mL) and 5% citric acid solution (20 mL). The aqueous layer was further extracted with CH₂Cl₂ (2×15 mL). The combined organic extract was washed with saturated NaCl solution, dried over anhydrous Na₂SO₄ and filtered. The solvent was removed under reduced pressure and the residue obtained was purified by silica column chromatography (30% EtOAc:n-hexanes) to yield androst-5-en-17-one-3β-yl succinate-propan-2-yl diester (113 mg, 76%) as a white oily solid.

¹H NMR (400 MHz, CDCl₃) δ 5.39 (d, J=5.1 Hz, 1H), 5.01 (hept, J=6.2 Hz, 1H), 4.67-4.54 (m, 1H), 2.63-2.54 (m, 4H), 2.48-0.98 (m, 19H), 1.22 (d, J=6.3 Hz, 6H), 1.03 (s, 3H), 0.87 (s, 3H); ¹³C NMR (101 MHz, CDCl₃) δ 221.2, 171.9, 171.9, 140.0, 122.0, 74.1, 68.1, 51.8, 50.2, 47.6, 38.1, 37.0, 36.8, 36.0, 31.6, 31.5, 30.9, 29.7, 29.6, 27.8, 22.0, 21.9, 20.4, 19.5, 13.7; LRMS (ESI+) found 453.3 m/z [M+Na]⁺; HRMS (ESI+) found m/z 452.2616 [M+Na]⁺, theoretical (C₂₆H₃₈O₅Na) m/z 452.2617 [M+Na]⁺; IR 2941, 1728 cm⁻¹.

### Testosterone-17β-yl hemisuccinate

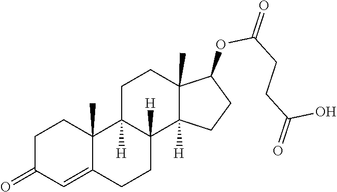

Testosterone (0.200 g, 0.694 mmol) in anhydrous toluene (8 mL) was treated as per general procedure C for 24 hours. The reaction mixture was diluted with CH₂Cl₂ (20 mL), water (20 mL) and brought to pH 2 by addition of 2M HCl solution. The aqueous layer was further extracted with CH₂Cl₂ (2×20 mL). The combined organic extract was washed with saturated NaCl solution, dried over anhydrous MgSO₄ and filtered. The solvent was removed under reduced pressure and the residue obtained was purified by silica column chromatography (2% AcOH:40% EtOAc:n-hexanes) to yield testosterone-17f-yl hemisuccinate xx (0.210 g, 78%) as a white oily solid.

m.p 169-171° C.; ¹H NMR (400 MHz, CDCl₃) δ 11.19-9.22 (m, 1H), 5.73 (s, 1H), 4.68-4.54 (m, 1H), 2.70-2.58 (m, 4H), 2.46-0.90 (m, 19H), 1.18 (s, 3H), 0.82 (s, 3H); ¹³C NMR (101 MHz, CDCl₃) δ 200.0, 177.6, 172.2, 171.5, 124.0, 83.0, 53.8, 50.3, 42.7, 38.7, 36.7, 35.8, 35.5, 34.0, 32.9, 31.6, 29.2, 29.1, 27.5, 23.6, 20.6, 17.5, 12.1; LRMS (ESI−) found m/z 387.2 [M−H]⁻; HRMS (ESI+) found m/z 411.2149 [M+Na]⁺, theoretical (C₂H₃₂O₅Na) m/z 411.2147 [M+Na]⁺; IR 3053, 2971, 2945, 2872, 1724, 1642, 1607 cm⁻¹.

### Testosterone-17β-yl succinate-primaquine amide

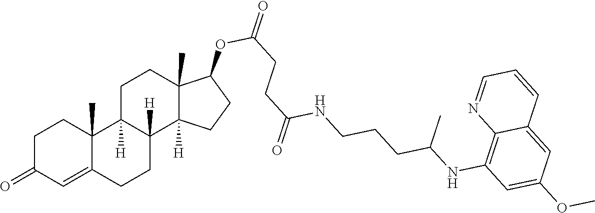

Testosterone-17β-yl hemisuccinate (34 mg, 0.089 mmol) in CH₂Cl₂ was treated as per general procedure D for 23 hours. The reaction mixture was diluted with CH₂Cl₂ (15 mL), water (10 mL) and 5% citric acid solution until pH 4. The aqueous layer was further extracted with CH₂Cl₂ (2×15 mL). The combined organic extract was washed with saturated NaHCO₃ solution and saturated NaCl solution, dried over anhydrous Na₂SO₄ and filtered. The solvent was removed under reduced pressure and the residue obtained was purified by silica column chromatography (3% MeOH:CH₂Cl₂) to yield testosterone-17β-yl succinate-primaquine amide (26 mg, 46%) as a yellow oil.

¹H NMR (400 MHz, MeOD) δ 8.51-8.43 (m, 1H), 7.99 (d, J=8.2 Hz, 1H), 7.98-7.92 (m, 1H), 7.37-7.29 (m, 1H), 6.45-6.39 (m, 1H), 6.33-6.25 (m, 1H), 5.68 (s, 1H), 4.58-4.48 (m, 1H), 3.89-3.81 (m, 3H), 3.67-3.56 (m, 1H), 3.27-3.15 (m, 2H), 2.65-2.56 (m, 2H), 2.45 (t, J=6.8 Hz, 2H), 2.42-0.68 (m, 23H), 1.27 (d, 3H), 1.14-1.10 (m, 3H), 0.80-0.76 (m, 3H), aniline NH not observed; ¹³C NMR (101 MHz, MeOD) δ 202.2, 174.9, 174.8, 174.1, 161.0, 146.2, 145.3, 136.5, 136.3, 131.6, 124.2, 123.0, 98.4/98.3, 93.04/93.02, 84.0, 55.7, 55.03/49.99, 51.26/51.18, 49.1, 43.7/43.7, 40.41/40.39, 39.9, 37.8/37.7, 36.6, 36.44/36.43, 35.1/35.0, 34.7, 33.8, 32.62/32.59, 31.6, 30.69/30.67, 28.3, 27.3/27.2, 24.3, 21.5, 20.6, 17.6, 12.4; LRMS (ESI+) found m/z 630.4 [M+H]⁺; HRMS (ESI+) found m/z 652.3726 [M+Na]⁺, theoretical (C₃₈H₅₁N₃O₅Na) m/z 652.3726 [M+Na]⁺; IR 3384, 2936, 1731, 1667, 1615, 1577, 1519 cm⁻¹.

### Testosterone-17β-yl succinate-propan-2-yl diester

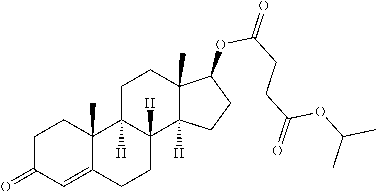

Testosterone (100 mg, 3.47 mmol) in CH₂Cl₂ was treated as per general procedure F overnight. The reaction was diluted with CH₂Cl₂ (20 mL) and 5% citric acid solution (20 mL). The aqueous layer was further extracted with CH₂Cl₂ (2×20 mL). The combined organic extract was washed with saturated NaCl solution, dried over anhydrous Na₂SO₄ and filtered. The solvent was removed under reduced pressure and the residue obtained was purified by silica column chromatography (30% EtOAc:n-hexanes) to yield testosterone-17β-yl succinate-propan-2-yl diester (114 mg. 76%) as white oily solid.

¹H NMR (400 MHz, CDCl₃) δ 5.72 (s, 1H), 5.01 (hept, J=6.2 Hz, 1H), 4.61 (t, J=8.4 Hz, 1H), 2.63-2.55 (m, 4H), 2.46-0.90 (m, 19H), 1.23 (d, J=6.2 Hz, 6H), 1.18 (s, 3H), 0.83 (s, 3H); ¹³C NMR (101 MHz, CDCl₃) δ 199.6, 172.4, 171.9, 171.1, 124.1, 82.8, 68.2, 53.8, 50.4, 42.7, 38.7, 36.7, 35.8, 35.5, 34.1, 32.9, 31.6, 29.7, 29.5, 27.6, 23.6, 21.9 (2C), 20.7, 17.5, 12.2; LRMS (ESI+) found 883.6 m/z [2M+Na]⁺; HRMS (ESI+) found m/z 453.2616 [M+Na]⁺, theoretical (C₂₆H₃₈O₅Na) m/z 453.2617 [M+Na]⁺; IR 2973, 2938, 1728, 1673, 1615 cm⁻¹.

### Propan-2-yl hemisuccinate

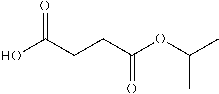

Procedure adapted from Cole, K. P.; Ryan, S. J.; Groh, J. M.; Miller, R. D. Reagent-Free Continuous Thermal Tert-Butyl Ester Deprotection. Org. Synth. Flow Med. Chem. 2017, 25 (23), 6209-6217. Succinic anhydride (2.00 g, 20.0 mmol) was added to a stirring solution of pyridine (2.0 mL, 25 mmol) in propan-2-ol (20 mL). The solution was heated to reflux and stirred for 20 h. The reaction mixture was concentrated under reduced pressure. The residue obtained was partitioned between CH₂Cl₂ (30 mL) and water (40 mL), then treated with 2M HCl solution until pH=2. The aqueous layer was further extracted with CH₂Cl₂ (2×30 mL). The combined organic extract was washed with saturated NaCl solution (60 mL), dried over anhydrous MgSO₄ and filtered. The solvent was removed under reduced pressure and the residue obtained was purified by silica column chromatography (20% EtOAc:n-hexanes) to yield propan-2-yl hemisuccinate xx (2.38 g, 74%) as a white oily solid.

m.p 52-53° C.; ¹H NMR (400 MHz, CDCl₃) δ 11.09 (s, 1H), 5.02 (hept, J=6.3 Hz, 1H), 2.67 (t, J=6.5 Hz, 2H), 2.58 (t, J=6.5 Hz, 2H), 1.23 (d, J=6.3 Hz, 6H); ³C NMR (101 MHz, CDCl₃) δ 178.3, 171.6, 68.3, 29.2, 29.0, 21.7; LRMS (ESI−) found m/z 159.1[M−H⁻.

### Propan-2-yl succinate-primaquine amide (Prim-Link)

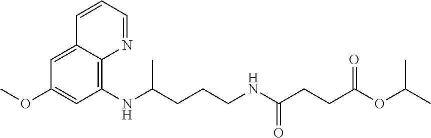 [SMILES: COc1cc(NC(C)CCCNC(=O)CCC(=O)OC(C)C)c2ncccc2c1]

Propan-2-yl hemisuccinate (40 mg, 0.25 mmol) in CH₂Cl₂ (5 mL) was treated as per general procedure D for 2 days. The reaction mixture was diluted with water (20 mL) and 5% citric acid solution until pH 5. The aqueous layer was further extracted with CH₂Cl₂ (2×30 mL). The combined organic extract was washed with saturated NaHCO₃ solution and saturated NaCl solution, dried over anhydrous Na₂SO₄ and filtered. The solvent was removed under reduced pressure and the residue obtained was purified by silica column chromatography (5% MeOH:CH₂Cl₂) to yield propan-2-yl succinate-primaquine amide (xx) (70 mg, 70%) as a green oil.

¹H NMR (400 MHz, MeOD) δ 8.48 (d, J=4.2 Hz, 1H), 8.01 (d, J=8.2 Hz, 1H), 7.37-7.32 (m, 1H), 6.47-6.41 (m, 1H), 6.33-6.28 (m, 1H), 4.94 (dq, J=12.5, 6.3 Hz, 1H), 3.86 (s, 3H), 3.69-3.59 (m, 1H), 3.23-3.13 (m, 2H), 2.54 (t, J=6.8 Hz, 2H), 2.43 (t, J=6.9 Hz, 2H), 1.74-1.58 (m, 4H), 1.28 (d, J=6.3 Hz, 3H), 1.18 (d, J=6.3 Hz, 6H); ¹³C NMR (101 MHz, MeOD) δ 174.3, 173.8, 161.0, 146.2, 145.3, 136.5, 136.3, 131.6, 122.9, 98.3, 93.0, 69.2, 55.6, 48.9, 40.3, 34.9, 31.5, 30.8, 27.1, 22.0, 20.8; LRMS (ESI+) found m/z 424/2 [M+Na]⁺; HRMS (ESI+) found m/z 402.2387 [M+H]⁻, theoretical (C₂₂H₃₂N₃O₄) m/z 402.2393 [M+H]⁺; IR 3390, 2981, 2863, 2472, 2414, 1727, 1634, 1615, 1594, 1576, 1518 cm⁻¹.

### Cholester-3-yl succinate-primaquine amide (C-3-Prim)

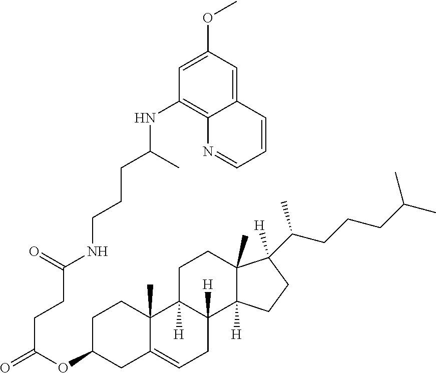

Cholester-3-yl hemisuccinate (50 mg, 0.10 mmol) in CH₂Cl₂ (5 mL) was treated as per general procedure D for 24 hours. The reaction mixture was diluted with CH₂Cl₂ (20 mL), water (20 mL) and 5% citric acid solution until pH 5. The combined organic extract was washed with saturated NaHCO₃ solution and saturated NaCl solution, dried over anhydrous Na₂SO₄ and filtered. The solvent was removed under reduced pressure and the residue obtained was purified by silica column chromatography (3% MeOH:CH₂Cl₂) to yield cholester-3-yl succinate-primaquine amide (57 mg, 76%) as a green oil.

¹H NMR (400 MHz, CD₂Cl₂) δ 8.54-8.47 (m, 1H), 7.97-7.90 (m, 1H), 7.31 (dd, J=8.2, 4.2 Hz, 1H), 6.35 (d, J=2.5 Hz, 1H), 6.27 (d, J=2.5 Hz, 1H), 6.02 (d, J=8.1 Hz, 1H), 5.82-5.72 (m, 1H), 5.35 (d, J=4.7 Hz, 1H), 4.60-4.49 (m, 1H), 3.87 (s, 3H), 3.67-3.57 (m, 1H), 3.31-3.15 (m, 2H), 2.56 (t, J=6.8 Hz, 2H), 2.38 (t, J=6.8 Hz, 2H), 2.29 (d, J=7.7 Hz, 2H), 1.28 (d, J=6.4 Hz, 3H), 1.00 (s, 3H), 0.92 (d, J=6.5 Hz, 3H), 0.87 (dd, J=6.6, 1.5 Hz, 6H), 0.68 (s, 3H); ¹³C NMR (101 MHz, CD₂Cl₂) δ 172.7, 171.6, 159.9, 145.4, 144.7, 140.2, 135.7, 135.1, 130.3, 122.9, 122.3, 97.0, 92.0, 74.6, 57.1, 56.6, 55.5, 50.8, 48.2, 42.7, 40.2, 39.9, 39.8, 38.5, 37.4, 37.0, 36.6, 36.2, 34.3, 32.3, 32.3, 31.4, 30.3, 28.6, 28.4, 28.1, 26.8, 24.6, 24.2, 23.0 (2C), 22.7, 21.4, 20.7, 19.5, 18.9, 12.0; LRMS (ESI+) found m/z 728.6 [M+H]⁺; HRMS (ESI+) found m/z 728.5364 [M+H]⁺, theoretical (C₄₆H₇₀N₃O₄) m/z 728.5366 [M+H]⁺; IR 3379, 2936, 2463, 1732, 1635, 1618, 1577, 1520 cm⁻¹.

### 2-Methoxyacetate-primaquine amide

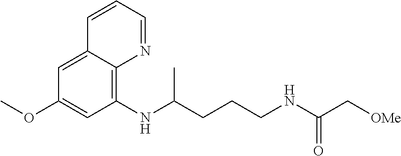 [SMILES: COCC(=O)NCCCC(C)Nc1cc(OC)cc2cccnc12]

Methoxyacetic acid (6.0 μL, 0.055 mmol) in CH₂Cl₂ was treated as per general procedure D* for 4 hours. The reaction mixture was diluted with water (10 mL) and 5% citric acid solution until pH 5. The aqueous layer was further extracted with CH₂Cl₂ (2×20 mL). The combined organic extract was washed with saturated NaHCO₃ solution and saturated NaCl solution, dried over anhydrous MgSO₄ and filtered. The solvent was removed under reduced pressure and the residue obtained was purified by silica column chromatography (10% MeOH:CH₂Cl) to yield 2-methoxyacetate-primaquine amide (8 mg, 44%) as a green oil. *Reaction conducted without the addition of HOBt

¹H NMR (600 MHz, MeOD δ 8.50-8.46 (m, 1H), 8.02 (d, J=8.4 Hz, 1H), 7.35 (dd, J=8.3, 4.1 Hz, 1H), 6.47-6.43 (m, 1H), 6.33-6.29 (m, 1H), 3.87 (s, 3H), 3.84 (s, 2H), 3.70-3.62 (m, 1H), 3.36 (s, 3H), 3.29-3.24 (m, 2H), 1.74-1.61 (m, 4H), 1.29 (d, J=6.3 Hz, 3H). ¹³C NMR (151 MHz, MeOD) δ 172.5, 161.0, 146.1, 145.3, 136.5, 136.3, 131.6, 122.9, 98.4, 93.0, 72.6, 59.5, 55.6, 48.9, 39.8, 34.8, 27.1, 20.7; LRMS (ESI+) found m/z 332.2 [M+H]⁺; HRMS (ESI+) found m/z 332.1977 [M+H]⁺, theoretical (C₁₈H₂₆N₃O₃) m/z 332.1974 [M+H]⁺.

### 2-(3β-(Tert-butyldimethylsilyloxy)-androst-5-en-17β-yloxy)-ethyl para-toluenesulfonate

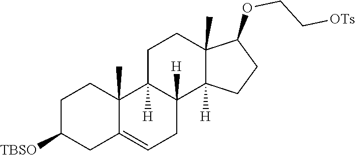

Procedure adapted from Hatakeyma et al., (2010).^{8A} 3β-(Tert-butyldimethylsilyloxy)-17β-(2-hydroxyethoxy)-androst-5-ene (68 mg, 0.15 mmol), pyridine (120 μL, 1.5 mmol), DMAP (19 mg, 0.16 mmol) and tosyl chloride (30 mg, 0.16 mmol) in CH₂Cl₂ (5 mL) were stirred at room temperature overnight. The reaction was poured over water (20 mL), brought to pH 2 with 2M HCl solution and extracted with CH₂Cl₂ (3×15 mL). The combined organic extract was washed with saturated NaCl solution, dried over anhydrous MgSO₄ and filtered. The solvent was removed under reduced pressure and the residue obtained was purified by silica column chromatography (5% EtOAc:n-hexanes) to yield 2-(3β-(ter-butyldimthylsilyloxy)-androst-5-en-17β-yloxy)-ethyl para-toluenesulfonate (64 mg, 70%) as a white oily solid.

¹H NMR (400 MHz, CDCl₃) δ 7.80 (d, J=8.0 Hz, 2H), 7.33 (d, J=8.0 Hz, 2H), 5.30 (d, J=5.1 Hz, 1H), 4.12 (t, J=5.0 Hz, 2H), 3.69-3.59 (m, 2H), 3.47 (m, 1H), 3.26 (t, J=8.3 Hz, 1H), 2.44 (s, 3H), 2.29-0.93 (m, 19H), 0.99 (s, 3H), 0.88 (s, 9H), 0.68 (s, 3H), 0.05 (s, 6H); ¹³C NMR (101 MHz, CDCl₃) δ 144.8, 141.8, 133.3, 129.9, 128.1, 120.9, 89.8, 72.7, 69.8, 67.5, 51.6, 50.4, 43.0, 42.9, 37.8, 37.5, 36.8, 32.2, 31.9, 31.6, 27.9, 26.1, 23.5, 21.8, 20.8, 19.6, 18.4, 11.5, −4.4 (2C); LRMS (ESI+) found m/z 625.3 [M+Na]⁺; HRMS (ESI+) found m/z 625.3336 [M+Na]⁺, theoretical (C₃₄H₅₄O₅SSiNa) m/z 625.3359 [M+Na]⁺; IR 2939, 1605 cm⁻¹.

### 2-(3β-(Tet-butyldimethylsilyloxy)-5α-androstan-17β-yloxy)-ethyl para-toluenesulfonate

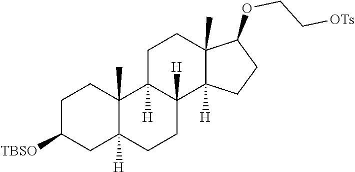

Procedure adapted from Hatakeyma et al., (2010).^{18A} 3β-(Tert-butyldimethylsilyloxy)-17β-(2-hydroxyethoxy)-5α-androstane (68 mg, 0.15 mmol), pyridine (120 μL, 1.5 mmol), DMAP (19 mg, 0.16 mmol) and tosyl chloride (30 mg, 0.16 mmol) in CH₂Cl₂ (5 mL) were stirred at room temperature overnight. The reaction was poured over water (20 mL), brought to pH 2 with 2M HCl solution and extracted with CH₂Cl₂ (3×15 mL). The combined organic extract was washed with saturated NaCl solution, dried over anhydrous MgSO₄ and filtered. The solvent was removed under reduced pressure and the residue obtained was purified by silica column chromatography (10% EtOAc:n-hexanes) to yield 2-(3β-(tert-butyldimethylsilyloxy)-5α-androstan-17β-yloxy)-ethyl para-toluenesulfonate (41 mg, 45%) as a white oily solid.

¹H NMR (400 MHz, CDCl₃) δ 7.79 (d, J=8.3 Hz, 2H), 7.33 (d, J=8.0 Hz, 2H), 4.11 (t, J=5.0 Hz, 2H), 3.68-3.58 (m, 2H), 3.57-3.50 (m, 1H), 3.24 (t, J=8.3 Hz, 1H), 2.44 (s, 3H), 1.94-0.83 (m, 21H), 0.88 (s, 9H), 0.79 (s, 3H), 0.65 (s, 3H), 0.60-0.55 (m, 1H), 0.04 (s, 6H); ¹³C NMR (101 MHz, CDCl₃) δ 144.8, 133.3, 129.9, 128.1, 89.9, 72.3, 69.8, 67.5, 54.7, 51.3, 45.2, 43.2, 38.8, 38.0, 37.4, 35.7, 35.4, 32.1, 31.8, 28.8, 28.0, 26.1, 23.4, 21.8, 21.0, 18.4, 12.5, 11.7, −4.4 (2C); LRMS (ESI+) found m/z 627.4 [M+Na]⁺; HRMS (ESI+) found m/z 627.3517 [M+Na]⁺, theoretical (C₃₄H₅₆O₅SSiNa) m/z 627.3515 [M+Na]⁺; IR 2928, 2855, 1598, 1363 cm⁻¹.

### 17β-(2-(Tert-butylperoxy)-ethoxy)-5α-androstan-3β-od

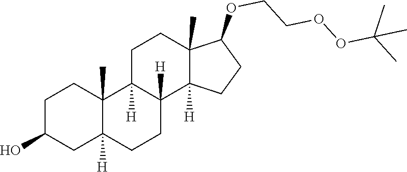

Procedure adapted from Dussault et al., (2000).^{19A} CsOH monohydrate (30 mg, 0.18 mmol) in anhydrous DMF (1 mL) under N₂ was cooled over an ice bath. Tert-butyl hydroperoxide (˜5.9 mg, ˜0.065 mmol) (nominally 5.5M in decane) was added dropwise and stirred for 30 minutes. 2-(3β-(Tert-butyldimethylsilyloxy)-5α-androstan-17β-yloxy)-ethyl para-toluenesulfonate (22 mg, 0.036 mmol) was added to the stirring solution. The reaction was brought to room temperature and stirred under N₂ overnight. The reaction mixture was diluted with water (20 mL) and extracted with CH₂Cl₂ (3×20 mL). The combined organic extract was washed saturated NaCl solution, dried over anhydrous Na₂SO₄ and filtered. The solvent was removed under reduced pressure and the residue obtained was purified by silica column chromatography (100% hexane, followed by 10% EtOAc:n-hexanes) to yield 17β-(2-(tert-butylperoxy)-ethoxy)-3β-(tert-butyldimethylsilyloxy)-5α-androstane (12 mg, 0.023 mmol) which was used without further purification. 17β-(2-(tert-butylperoxy)-ethoxy)-3β-(tert-butyldimethylsilyloxy)-Sa-androstane and CSA (1 mg, 0.4 μmol) in MeOH/CH₂Cl₂ (2 mL) were treated a per general procedure E for 2 hours. The reaction was quenched with saturated NaHCO₃ and diluted with water (5 mL). The aqueous layer was extracted with CH₂Cl₂ (2×15 mL). The combined organic extract was washed with saturated NaCl solution, dried over anhydrous Na₂SO₄ and filtered. The solvent was removed under reduced pressure and the residue obtained was purified by silica column chromatography (5% EtOAc:n-hexanes) to yield 17β-(2-(tert-butylperoxy)-ethoxy)-5α-androstan-3β-ol (8.1 mg, 55% over 2 steps) as a clear oil.

¹H NMR (400 MHz, CDCl₃) δ 4.04 (t, J=5.1 Hz, 2H), 3.68-3.55 (m, 3H), 3.32 (t, J=8.2 Hz, 1H), 2.02-0.83 (m, 21H), 1.24 (s, 9H), 0.81 (s, 3H), 0.74 (s, 3H), 0.65-0.58 (m, 1H) OH not observed; ¹³C NMR (101 MHz, CDCl₃) δ 89.7, 80.4, 74.8, 71.5, 67.4, 54.6, 51.4, 45.1, 43.3, 38.4, 38.2, 37.2, 35.7, 35.5, 31.8, 31.7, 28.8, 28.2, 26.5, 23.5, 21.1, 12.5, 11.8; LRMS (ESI+) found m/z 431.4 [M+Na]⁺, HRMS (ESI+) m/z 431.3148 [M+Na]⁺, theoretical (C₂₅H₄₄O₄Na) m/z 431.3137 [M+Na]⁺. IR 3332, 2973, 2927, 2855 cm⁻¹.

### Cholester-3-yl hemisuccinate

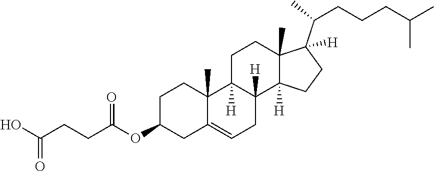

Procedure adapted from Kumar, P.; Shankar Rao. D. S.; Krishna Prasad, S.; Jayaraman, N. In-Plane Modulated Smectic Ā vs Smectic ‘A’ Lamellar Structures in Poly(Ethyl or Propyl Ether Imine) Dendrimers. Polymer 2016, 86, 98-104. Cholesterol (773.4 mg, 2 mmol) and succinic anhydride (320.1 mg, 3.2 mmol) were dissolved in dry toluene (4 mL). Triethylamine (70 μL, 0.5 mmol) was added to the suspension and the reaction mixture was stirred at 60° C. overnight. The reaction mixture was cooled down to ambient temperature and the pale yellow suspension was treated with water (10 mL) and the organic layer extracted with dichloromethane (2×20 mL). The combined organic extracts were washed with 2 M hydrochloric acid (3×5 mL), water (3×5 mL) and saturated brine solution (8 mL) before being dried over anhydrous (Na₂SO₄). The solution was then dried in vacuo to afford cholester-3-yl hemisuccinate (758.2 mg. 72%), as a white solid.

¹H NMR (400 MHz, CDCl₃) δ 5.37 (d, J 5.1 Hz, 1H), 4.63 (m, 1H), 2.64 (m, 4H), 2.32 (d, J 8.0 Hz, 2H), 2.03-1.76 (m, 5H), 1.62-1.02 (m, 18H), 1.00 (s, 3H), 0.98-0.92 (m, 3H), 0.89 (d, J 6.5 Hz, 3H), 0.84 (dd, J 6.6, 1.8 Hz, 6H), 0.65 (s, 3H); LRMS (ESI+) found m/z 509 [M+Na]⁺; IR (ATR, solid) 2937, 1707, 1465, 1435, 1377, 1316, 1247, 1180, 1000, 942, 799, 736, 655 cm⁻¹.

### 3β-Succinate-(propan-2-yl diester)-cholest-5-ene (C-3-Link)

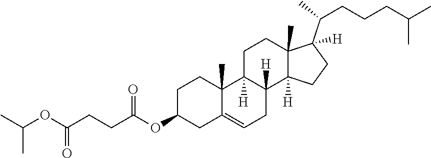

A solution of cholesterol hemisuccinate (20.1 mg, 0.041 mmol) and EDC·HCl (15.8 mg, 0.082 mmol) in dry dichloromethane (1 mL) was stirred at 0° C. for 5 minutes under N₂. To the solution, DMAP (9.9 mg, 0.082 mmol), isopropanol (6.3 μL, 0.082 mmol) and additional dichloromethane (1 mL) were added. The reaction mixture was warmed up to ambient temperature and left to stir under N₂ overnight. The solution was then quenched by dropwise addition of 5% citric acid solution (5 mL). The pale yellow solution was then extracted with ethyl acetate (3×5 mL) to give a colourless organic extract. The combined organic extracts were then washed with saturated brine solution (10 mL) and dried over anhydrous MgSO₄. The crude material was purified by flash chromatography (silica, 1:3 ethyl acetate:hexanes) to afford 3β-succinate-(propan-2-yl diester)-cholest-5-ene (11.4 mg, 52%) as a white solid.

¹H NMR (400 MHz, CDCl₃) δ 5.35 (d, J 4.05 Hz, 1H), 4.99 (sept., J 6.3 Hz, 1H), 4.60 (m, 1H), 2.56 (s, 4H), 2.29 (d, J 7.8 Hz, 2H), 2.02-1.76 (m, 5H), 1.61-1.25 (m, 12H), 1.21 (d, J 6.3 Hz, 6H), 1.17-1.02 (m, 7H), 0.99 (s, 3H), 0.96-0.91 (m, 2H), 0.89 (d, J 6.5 Hz, 3H), 0.84 (dd, J 6.6, 1.8 Hz, 6H), 0.65 (s, 3H); ¹³C NMR (175 MHz, CDCl₃) δ 172.1, 171.9, 139.8, 122.9, 74.5, 68.2, 56.9, 56.4, 50.2, 42.5, 39.9, 39.7, 38.3, 37.2, 36.8, 36.4, 36.0, 32.1, 32.1, 29.8, 29.8, 28.4. 28.2, 28.0, 24.5, 24.0, 23.0, 22.8, 22.0, 21.2, 19.5, 18.9, 12.1; LRMS (ESI+) found m/z 551 [M+Na]⁺; HRMS (ESI+) found m/z 529.4266 [M+H]⁺, theoretical (C₃₄H₅₇O₄) m/z 529.4251 [M+H]⁺; IR (ATR, solid) 2941, 2890, 2869, 2852, 1728, 1467, 1373, 1318, 1309, 1164, 1107, 996, 982, 958 cm⁻¹; Specific rotation [α]_{D}²⁵−24.78 (c 1.0, CHCl₃).

### 3β-Artesunate ester-cholest-5-ene

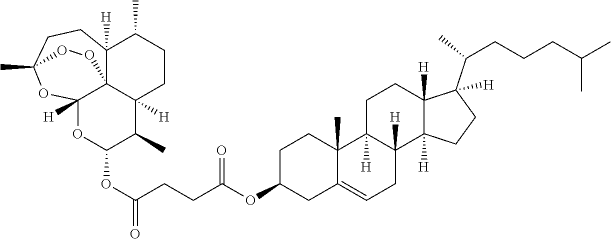

A solution of artesunate (20 mg, 0.052 mmol) and EDC·HCl (19.9 mg, 0.104 mmol) in dry dichloromethane (1 mL) was stirred at 0° C. for 5 minutes under N₂. To the solution, DMAP (12.7 mg, 0.104 mmol), cholesterol (40.2 mg, 0.104 mmol) and additional dichloromethane (1 mL) were added. The reaction mixture was warmed up to ambient temperature and left to stir under N₂ for 24 h. The solution was then quenched by dropwise addition of 5% citric acid solution (5 mL). The colourless solution was then extracted with ethyl acetate (3×5 mL) to give a colourless organic extract. The extract was then washed with saturated brine solution (10 mL) and dried over anhydrous MgSO₄. The crude material was purified by flash chromatography (silica, 1:3 ethyl acetate:hexanes) to afford 3β-artesunate ester-cholest-5-ene (19.7 mg, 50%) as a white solid.

¹H NMR (400 MHz, CDCl₃) δ 5.77 (d, J 9.8 Hz, 1H), 5.41 (s, 1H), 5.34 (d, J 4.5 Hz, 1H), 4.60 (m, 1H), 2.72-2.27 (m, 8H), 2.02-1.43 (m, 20H), 1.40 (s, 3H), 1.37-1.01 (m, 16H), 0.99 (s, 3H), 0.94 (d, J 5.9 Hz, 3H), 0.89 (d, J 6.5 Hz, 3H), 0.84 (m, 6H), 0.83 (d, J 7.1 Hz, 3H), 0.65 (s, 3H); ¹³C NMR (175 MHz, CDCl₃) δ 171.7, 171.4, 139.8, 122.9, 104.7, 92.3, 91.7, 80.3, 74.6, 56.9, 56.4, 51.8, 50.2, 45.5, 42.5, 39.9, 39.7, 38.3, 37.5, 37.2, 36.8, 36.4, 36.4, 36.0, 34.3, 32.1, 32.0, 29.5, 29.4, 28.4, 28.2, 27.9, 26.2, 24.8, 24.5, 24.0, 23.1, 22.8, 22.2, 21.2, 20.4, 19.5, 19.0, 12.3, 12.1; LRMS (ESI+) found m/z 776 [M+Na]⁺; HRMS (ESI+) found m/z 775.5128 [M+Na]⁺, theoretical (C₄₆H₇₂O₈Na) m/z 775.5119 [M+Na]⁺; IR (ATR, solid) 2935, 2869, 1734, 1466, 1455, 1376, 1364, 1253, 1201, 1157, 1133, 1102, 1016, 926, 912, 877, 845, 826, 732 cm⁻¹; Specific rotation [α]_{D}²⁵−12.86 (c 1.0, CHCl₃).

### Propan-2-yl artesunate ester (Art-Link)

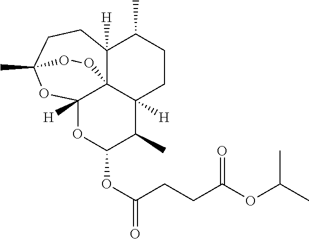 [SMILES: CC(C)OC(=O)CCC(=O)O[C@@H]1O[C@@H]2O[C@]3(C)CC[C@H]4[C@H](C)CC[C@@H]([C@H]1C)[C@@]24OO3]

A solution of artesunate (20 mg, 0.052 mmol) and EDC·HCl (19.9 mg, 0.104 mmol) was stirred in dry dichloromethane (1 mL) at 0° C. for 5 minutes under N₂. To the solution, DMAP (12.7 mg, 0.104 mmol), isopropanol (7.78 μL, 0.104 mmol) and additional dichloromethane (1 mL) were added. The reaction mixture was warmed up to ambient temperature and left to stir under N₂ for 4 h. Additional isopropanol (7.78 μL, 0.054 mmol) was added and the reaction was stirred for another 2 h. The solution was then quenched by dropwise addition of 5% citric acid solution (5 mL). The colourless solution was then extracted with ethyl acetate (3×5 mL) to give a colourless organic extract. The extract was then washed with saturated brine solution (10 mL) and dried over anhydrous MgSO₄. The crude material was purified by flash chromatography (silica, 1:3 ethyl acetate:hexanes) to afford propan-2-yl artesunate ester (9.4 mg, 42%) as a white solid.

¹H NMR (400 MHz, CDCl₃) δ 5.77 (d, J 9.9 Hz, 1H), 5.41 (s, 1H), 4.99 (sept., J 6.2 Hz, 1H), 2.71-2.50 (m, 5H), 2.35 (td, J 14.0, 4.0 Hz, 1H), 2.00 (ddd, J=14.6, 4.7, 3.1 Hz, 1H), 1.81 (m, 1H), 1.72 (m, 2H), 1.62-1.57 (m, 1H), 1.51-1.43 (m, 1H), 1.40 (s, 3H), 1.37-1.23 (m, 3H), 1.20 (dd, J 6.3, 0.8 Hz, 6H), 1.06-0.96 (m, 1H), 0.94 (d, J 5.9 Hz, 3H), 0.83 (d, J 7.1 Hz, 3H); ¹³C NMR (175 MHz, CDCl₃) δ 171.8, 171.4, 104.7, 92.3, 91.7, 80.3, 68.3, 51.8, 45.5, 37.5, 36.5, 34.3, 32.0, 29.5, 29.5, 26.2, 24.8, 22.2, 22.0, 20.4, 12.3; LRMS (ESI+) found m/z 449 [M+Na]⁺; HRMS (ESI+) found m/z 449.2149 [M+Na]⁺, theoretical (C₂₂H₃₄O₈Na) m/z 449.2146 [M+Na]⁺; IR (ATR, solid) 2976, 2928, 2875, 1750, 1730, 1454, 1375, 1201, 1159, 1102, 1036, 1012, 926, 876, 825 cm⁻¹; Specific rotation [α]_{D}²⁵+11.18 (c 1.0, CHCl₃).

### 3β-(tert-butyldimethylsilyloxy)-androst-5-en-17β-yl succinate-artesunate ester

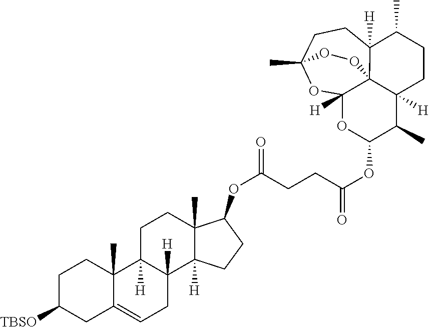

A solution of artesunate (10.9 mg, 0.028 mmol) and EDC·HCl (10 mg, 0.052 mmol) was stirred in dichloromethane (2 mL) in a flame dried round bottom flask under N₂. After 5 m a solution of DMAP (6.4 mg, 0.052 mmol) and 3β-(tert-butyldimethylsilyloxy)-androst-5-en-17β-ol (21.1 mg, 0.052 mmol) in dichloromethane (2 mL) was added. The combined solution was left stirring at room temperature for 20 h before being quenched with 5% citric acid solution (5 mL) and extracted with EtOAc (4×5 mL). The combined organic layers were washed with brine (10 mL) and dried over anhydrous MgSO₄ and finally concentrated under reduced pressure to give a crude white solid. The crude product was purified by flash chromatography (silica. 10% EtOAc in hexanes) to give 3β-(tert-butyldimethylsilyloxy)-androst-5-en-17β-yl succinate-artesunate ester (11.9 mg, 54%) as a clear oil.

¹H NMR (600 MHz, CDCl₃) δ 5.79 (d, J 9.9 Hz, 1H), 5.43 (s, 1H), 5.31 (d, J 5.3 Hz, 1H), 4.61 (dd, J 8.3, 8.9 Hz, 1H), 3.48 (m, 1H), 2.72 (m, 2H), 2.65 (m, 2H), 2.57 (m, 1H), 2.37 (ddd, J 4.66, 14.26, 13.62 Hz, 1H), 2.26 (m, 1H), 2.01 (m, 2H), 1.89 (m, 1H), 1.75 (m, 5H), 1.63 (m, 2H), 1.51 (m, 7H), 1.43 (s, 3H), 1.39 (m, 2H), 1.30 (m, 3H), 1.16 (m, 1H), 1.04 (m, 2H), 1.00 (s, 3H), 0.96 (d, J 6.20 Hz, 3H), 0.92 (m, 1H), 0.88 (s, 9H), 0.85 (d, J 7.20 Hz, 3H), 0.79 (s, 3H), 0.05 (s, 6H); ¹³C NMR (150 MHz, CDCl₃) δ 172.20, 171.30, 141.79, 120.89, 104.60, 92.24, 91.64, 83.28, 80.27, 51.72, 51.22, 50.29, 45.39, 42.95, 37.52, 37.43, 36.93, 36.79, 36.37, 34.24, 32.20, 31.94, 31.86, 31.64, 29.50, 29.28, 27.64, 26.12, 26.09, 24.73, 23.74, 22.15, 20.67, 20.37, 19.61, 18.42, 12.26, 12.16, −4.44; LRMS (ESI+) found m/z 794 [M+Na]⁺; HRMS (ESI+) found m/z 793.4686 [M+Na]⁺, theoretical (C₄₄H₇₀O₉SiNa) m/z 793.4687 [M+Na]⁺; Specific rotation [α]_{D}²⁵−14.36 (c 0.8, CHCl₃).

### 3β-Hydroxyandrost-5-en-17β-yl succinate-artesunate ester (C-17-art)

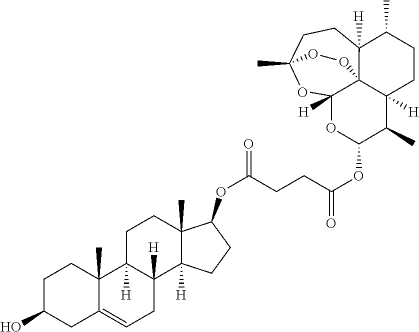

A solution of 3β-(tert-butyldimethylsilyloxy)-androst-5-en-17β-yl succinate-artesunate ester (30 mg, 0.039 mmol) in dry tetrahydrofuran (10 mL) was treated with 1M TBAF solution in THF (140 μL, 0.14 mmol) and stirred for 40 h at room temperature. The solvent was removed under reduced pressure to give a crude yellow oil. The crude material was purified by flash chromatography (silica, 1:1 ethyl acetate:hexanes) to afford 3β-hydroxyandrost-5-en-17β-yl succinate-artesunate ester (14.8 mg, 57%) as a colourless oil.

¹H NMR (400 MHz, CDCl₃) δ 5.79 (d, J 9.8 Hz, 1H), 5.43 (s, 1H), 5.34 (d, J 5.0 Hz, 1H), 4.61 (t, J 8.4 Hz, 1H) 3.52 (td, J 5.4, 11.2 Hz, 1H), 2.72-0.88 (m, 36H) 1.43, (s, 3H), 1.01 (s, 3H), 0.96 (d, J 6.2 Hz, 3H), 0.85 (d, J 7.2 Hz, 3H), 0.80 (s, 3H); ¹³C NMR (175 MHz, CDCl₃) δ 172.20, 171.30, 141.01, 121.43, 104.60, 92.26, 91.64, 83.27, 80.27, 71.85, 60.54, 51.73, 51.20, 50.21, 45.40, 42.60, 42.40, 37.43, 36.92, 36.71, 36.37, 34.25, 31.94, 31.86, 31.77, 31.60, 29.50, 29.28, 27.64, 26.10, 24.73, 22.15, 20.69, 20.36, 19.56, 14.34, 12.24, 12.16; LRMS (ESI+) found m/z 679 [M+Na]⁺; HRMS (ESI+) found m/z 679.3815 [M+Na]⁺, theoretical (C₃₈H₅₆O₉Na) m/z 679.3822 [M+Na]⁺; IR (ATR, solid) 3450, 2930, 1735, 1454, 1375, 1158, 1037, 1015, 877 cm⁻¹.

### 3β-(tert-butyldimethylsilyloxy)-androst-5-en-17β-yl succinate-chloromethyl ester

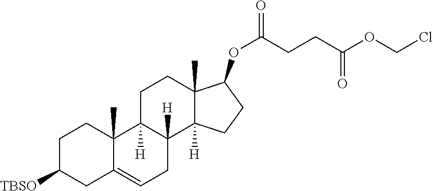

A solution of 3β-(tert-butyldimethylsilyloxy)-androst-5-en-17β-yl hemisuccinate (1.00 g, 1.98 mmol), potassium carbonate (1.10 g, 7.92 mmol) and tetrabutylammonium hydrogen sulfate (67 mg, 0.20 mmol) in water/DCM (20 mL) was treated with chloromethyl chlorosulfate (443 mg, 2.97 mmol) and stirred for 20 h at room temperature. The mixture was diluted with water (20 mL) and extracted with DCM (3×20 mL). The combined organic phases were washed with brine solution (1×50 mL), dried (MgSO₄) and concentrated under reduced pressure to afford a crude white solid. The crude product was purified by flash chromatography (silica, 20% EtOAc in hexanes) to give 3β-(tert-butyldimethylsilyloxy)-androst-5-en-17-yl succinate-chloromethyl ester (640 mg, 58%) as a colourless oil.

¹H NMR (400 MHz, CDCl₃) δ 5.71 (s, 2H), 5.31 (d, J 5.3 Hz, 1H), 4.63 (t, J 8.5 Hz, 1H), 3.47 (td, J 10.8, 5.4 Hz, 1H), 2.94-2.51 (m, 4H), 2.26-1.04 (m, 16H), 1.00 (s, 3H), 0.88 (s, 9H), 0.80 (s, 3H), 0.05 (s, 6H).

### 3β-(tert-butyldimethylsilyloxy)-androst-5-en-17β-yl succinate-(hydroxymethoxy)-resorufin ester

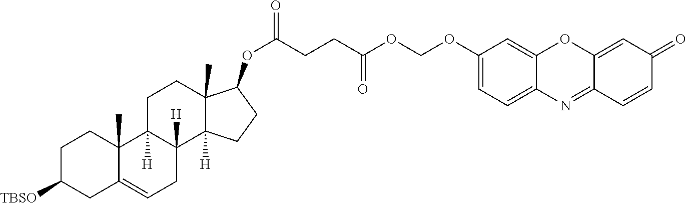

A solution of 3β-(tert-butyldimethylsilyloxy)-androst-5-en-17β-yl succinate-chloromethyl ester (200 mg. 0.361 mmol) in dry acetone (5 mL) was treated with sodium iodide (108 mg, 0.723 mmol) and stirred for 20 h at room temperature in a flask shielded from light. The solvent was removed under reduced pressure and the resulting yellow solid dissolved in DCM (10 mL) and filtered to remove insoluble material, the filtrate was evaporated under reduced pressure and the resultant yellow solid reconstituted in DCM (3 mL). This solution was then treated with a solution of resorufin (85 mg, 0.40 mmol), potassium carbonate (150 mg, 1.08 mmol) and tetrabutylammonium hydrogen sulfate (12 mg, 0.036 mmol) in water (3 mL) in a flask shielded from light and stirred at room temperature for 3 days. The mixture was then diluted with water (40 mL) and extracted with DCM (3×40 mL). The combined organic phases were washed with water (1×50 mL) and brine solution (1×50 mL), dried (Na₂SO₄) and concentrated under reduced pressure to give a crude brown solid.

The crude material was purified by flash chromatography (silica, ethyl acetate:hexanes) to afford 3β-(tert-butyldimethylsilyloxy)-androst-5-en-17β-yl succinate-(hydroxymethoxy)-resorufin ester (32 mg, 12%) as an orange solid.

¹H NMR (600 MHz, CDCl₃) δ 7.74 (d, J 8.8 Hz, 1H), 7.42 (d, J 9.8 Hz, 1H), 7.04 (d, J 11.4 Hz, 1H), 6.99 (s, 1H), 6.85 (d, J 7.9 Hz, 1H), 6.33 (s, 1H), 6.01-5.78 (m, 2H), 5.38-5.19 (m, 1H), 4.59 (t, J 8.4 Hz, 1H), 3.47 (dt, J 11.1, 5.6 Hz, 1H), 3.09-2.52 (m, 4H), 2.31-1.02 (m, 19H), 0.99 (s, 3H), 0.88 (s, 9H), 0.76 (s, 3H), 0.05 (m, 6H); ¹³C NMR (150 MHz, CDCl₃) δ 186.42, 171.96, 171.25, 160.28, 149.75, 146.90, 145.43, 141.78, 134.91, 134.81, 131.87, 129.54, 120.85, 114.48, 107.19, 102.84, 85.02, 83.51, 72.67, 51.20, 50.27, 49.79, 42.95, 42.63, 41.00, 37.53, 36.94, 36.78, 32.19, 31.87, 31.61, 29.26, 29.11, 27.63, 26.09, 26.07, 23.72, 20.69, 19.60, 18.40, 12.07, −4.43; LRMS (ESI+) found m/z 752 [M+Na]⁺; HRMS (ESI+) found m/z 752.35937 [M+Na]⁺, theoretical (C₄₂H₅₅NO₈SiNa) m/z 752.35892 [M+Na]⁺.

### 3α-Hydroxyandrost-5-en-17β-yl succinate-(hydroxymethoxy)-resorufin ester

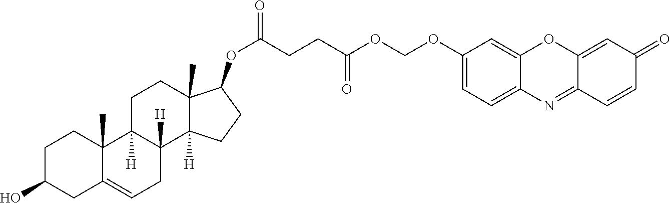

A solution of 3β-(tert-butyldimethylsilyloxy)-androst-5-en-17β-yl succinate-(hydroxymethoxy)-resorufin ester (10 mg, 0.014 mmol) in 1,4-dioxane (1.5 mL) was treated with conc. sulfuric acid (1 drop, excess) and stirred for 1 h at room temperature. The solvent was removed under reduced pressure and the resulting crude yellow oil diluted with water (10 mL) and extracted with DCM (3×10 mL). The combined organic phases were washed with water (1×5 mL) and brine solution (1×5 mL), dried (Na₂SO₄) and concentrated under reduced pressure to give a crude yellow solid. The crude material was purified by flash chromatography (silica, ethyl acetate:hexanes) to afford 3β-hydroxyandrost-5-en-17β-yl succinate-(hydroxymethoxy)-resorufin ester (3 mg, 34%) as a yellow solid.

¹H NMR (400 MHz, CDCl₃) δ 7.75 (d, J 8.8 Hz, 1H), 7.43 (d, J 9.9 Hz, 1H), 7.05 (dd, J 8.8, 2.6 Hz, 1H), 7.00 (d, J 2.5 Hz, 1H), 6.85 (m, 1H), 6.34 (d, J=2.0 Hz, 1H), 5.90-5.84 (m, 2H), 5.36-5.34 (m, 1H), 4.61-4.58 (m, 1H), 3.56-3.50 (m, 1H), 2.92-2.56 (m, 4H), 2.29-0.81 (m, 20H), 1.00 (s, 3H), 0.76 (s, 3H).

### 3β-(Tert-butyldimethylsilyloxy)-androst-5-en-17β-yl succinate-metronidazole diester

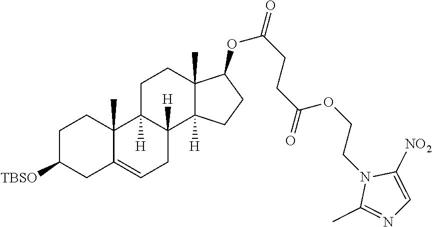

3β-(Tert-butyldimethylsilyloxy)-androst-5-en-17β-yl hemisuccinate (50 mg, 0.099 mmol) and metronidazole (25 mg, 0.15 mmol) in CH₂Cl₂ (5 mL) was treated as per general procedure G for 25 hours. The reaction mixture was diluted with CH₂Cl₂ (15 mL), water (20 mL) and 5% citric acid solution until pH 5. The aqueous layer was further extracted with CH₂Clₕ (3×20 mL). The combined organic extract was washed with saturated NaHCO₃ solution and saturated NaCl solution, dried over anhydrous Na₂SO₄ and filtered. The solvent was removed under reduced pressure and the residue obtained was purified by silica column chromatography (3% MeOH:CH₂Cl) to yield 3β-(tert-butyldimethylsilyloxy)-androst-5-en-17β-yl succinate-metronidazole diester (52 mg, 77%) as a colourless oil.

### 3β-Hydroxyandrost-5-en-17β-yl succinate-metronidazole diester (BC62D)

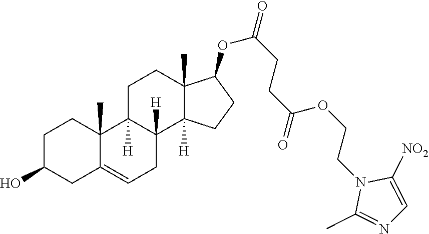

3β-(Tert-butyldimethylsilyloxy)-androst-5-en-17β-yl succinate-metronidazole diester (34 mg. 0.052 mmol) in MeOH/CH₂Cl₆ (4 mL) was treated as per general procedure E for 1 hour. The reaction was quenched with saturated NaHCO₃ and diluted with water (15 mL) and CH₂Cl₂ (15 mL). The aqueous layer was further extracted with CH₂Cl₂ (2×15 mL). The combined organic extract was washed with saturated NaCl solution, dried over anhydrous Na₂SO₄ and filtered. The solvent was removed under reduced pressure and the residue obtained was purified by silica column chromatography (3% MeOH:CH₂Cl₂) to yield 3β-hydroxyandrost-5-en-17β-yl succinate-metronidazole diester (27 mg, 96%) as a white solid.

### Propan-2-yl succinate-metronidazole amide

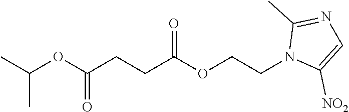 [SMILES: Cc1ncc([N+](=O)[O-])n1CCOC(=O)CCC(=O)OC(C)C]

Propan-2-yl hemisucciante (50 mg, 0.3 mmol) and metronidazole (80 mg, 0.47 mmol) in CH₂Cl₂ (5 mL) was treated as per general procedure G for 24 hours. The reaction mixture was diluted with CH₂Cl₂ (20 mL), water (20 mL) and 5% citric acid solution until pH 5. The aqueous layer was further extracted with CH₂Cl₂ (3×20 mL). The combined organic extract was washed with saturated NaHCO₃ solution and saturated NaCl solution, dried over anhydrous Na₂SO₄ and filtered. The solvent was removed under reduced pressure and the residue obtained was purified by silica column chromatography (3% MeOH:CH₂Cl₂) to yield propan-2-yl succinate-metronidazole amide (81 mg, 83%) as a colourless oil.

The following references relate to the description of the specific transformations in Section 1.3 above.
(1A) Fulmer, G. R.; Miller, A. J. M.; Sherden, N. H.; Gottlieb, H. E.; Nudelman, A.; Stoltz, B. M.; Bercaw, J. E.; Goldberg, K. I. NMR Chemical Shifts of Trace Impurities: Common Laboratory Solvents. Organics, and Gases in Deuterated Solvents Relevant to the Organometallic Chemist. Organometallics 2010, 29 (9), 2176-2179. https://doi.org/10.1021/om100106e.
(2A) Yamauchi. T.; Kato, M.; Mikami, T.; Fujimura, Y. Synthesis of 1-Deoxymaxacalcitol. Heterocycles 2005, 65 (9), 2111.
(3A) Moreira, V. M. A.; Vasaitis, T. S.; Guo, Z.; Njar, V. C. O.; Salvador, J. A. R. Synthesis of Novel C17 Steroidal Carbamates: Studies on CYP17 Action, Androgen Receptor Binding and Function, and Prostate Cancer Cell Growth. Steroids 2008, 73 (12), 1217-1227. https://doi.org/10.1016/j.steroids.2008.05.010.
(4A) András Keglevich; Vivian Zsiros; Péter Keglevich*; Áron Szigetvári; Miklós Dékány; Csaba Szántay; Erzsébet Mernyák; János Wölfling and László Hazai*. Synthesis and In Vitro Antitumor Effect of New Vindoline-Steroid Hybrids. Curr. Org. Chem. 2019, 23 (8), 959-967. https://doi.org/10.2174/1385272823666190614113218.
(5A) Fernandes, I.; Vale, N.; de Freitas, V.; Moreira, R.; Mateus, N.; Gomes, P. Anti-Tumoral Activity of Imidazoquines, a New Class of Antimalarials Derived from Primaquine. Bioorg. Med. Chem. Left. 2009, 19 (24), 6914-6917. https://doi.org/10.1016/j.bmcl.2009.10.081.
(6A) Yamashita, T.; Kawai, N.; Tokuyama, H.; Fukuyama, T. Stereocontrolled Total Synthesis of (−)-Eudistomin C. J. Am. Chem. Soc. 2005, 127 (43), 15038-15039. https://doi.org/10.1021/ja055832h.
(7A) Krausova, B.; Slavikova, B.; Nekardova, M.; Hubalkova, P.; Vyklicky, V.; Chodounska, H.; Vyklicky, L.; Kudova, E. Positive Modulators of the N-Methyl-d-Aspartate Receptor: Structure-Activity Relationship Study of Steroidal 3-Hemiesters. J. Med. Chem. 2018, 61 (10), 4505-4516. https://doi.org/10.1021/acs.jmedchem.8b00255.
(8A) Liu, D.; Stuhmiller, L. M.; McMorris, T. C. Synthesis of 15β-Hydroxy-24-Oxocholesterol and 15β,29-Dihydroxy-7-Oxofucosterol. J. Chem. Soc. Perkin 1 1988, No. 8, 2161-2167. https://doi.org/10.1039/P19880002161.
(9A) Calogeropoulou, T.; Avlonitis, N.; Minas, V.; Alexi, X.; Pantzou, A.; Charalampopoulos, I.; Zervou, M.; Vergou, V.; Katsanou, E. S.; Lazaridis, I.; Alexis, M. N.; Gravanis, A. Novel Dehydroepiandrosterone Derivatives with Antiapoptotic, Neuroprotective Activity. J. Med. Chem. 2009, 52 (21), 6569-6587. https://doi.org/10.1021/jm900468p.
(10A) Shen, Y.; Burgoyne, D. L. Efficient Synthesis of IPL576,092: A Novel Anti-Asthma Agent. J. Org. Chem. 2002, 67 (11), 3908-3910. https://doi.org/10.1021/jo0108717.
(11A) Upsani, R. B.; Fick, D. B.; Hogenkamp, D. J.; Lan, N. C. Neuroactive Steroids of the Androstane and Pregnane Series. U.S. Pat. No. 5,925,630A, Jul. 20, 1999.
(12A) Epp, J. B.; Widlanski, T. S. Facile Preparation of Nucleoside-5′-Carboxylic Acids. J. Org. Chem. 1999, 64 (1). 293-295. https://doi.org/10.1021/jo981316g.
(13A) Kratena, N.; Biedermann, N.; Stojanovic, B.; Göschl, L.; Weil, M.; Enev, V. S.; Gmeiner. G.; Gärtner, P. Synthesis of a Human Long-Term Oxymetholone Metabolite. Steroids 2019, 150, 108430. https://doi.org/10.1016fj.steroids.2019.108430.
(14A) Hosoda, H.; Fukushima, D. K.; Fishman. J. Convenient, High Yield Conversion of Androst-5-Ene-3.Beta.,17.Beta.-Diol to Dehydroisoandrosterone. J. Org. Chem. 1973, 38 (24), 4209-4211. https://doi.org/10.1021/jo00963a028.
(15A) Felzmann, W.; Gmeiner, G.; Gärtner, P. First Synthesis of a Pentadeuterated 3′-Hydroxystanozolol—an Internal Standard in Doping Analysis. Steroids 2005, 70 (2). 103-110. https://doi.org/10.1016/j.steroids.2004.10.002.
(16A) Klobucki, M.; Grudniewska, A.; Smuga, D. A.; Smuga, M.; Jarosz, J.; Wietrzyk, J.; Maciejewska, G.; Wawrzeńczyk, C. Syntheses and Antiproliferative Activities of Novel Phosphatidylcholines Containing Dehydroepiandrosterone Moieties. Steroids 2017, 118, 109-118. https://doi.org/10.1016/j.steroids.2016.12.015.
(17A) Jao, S.-C.; Chen, J.; Yang, K.; Li, W.-S. Design of Potent Inhibitors for Schistosoma Japonica Glutathione S-Transferase. Bioorg. Med. Chem. 2006, 14 (2), 304-318. https://doi.org/10.1016/j.bmc.2005.07.077.
(18A) Hatakeyama, S.; Yoshino, M.; Eto, K.; Takahashi, K.; Ishihara, J.; Ono. Y.; Saito, H.; Kubodera, N. Synthesis and Preliminary Biological Evaluation of 20-Epi-Eldecalcitol [20-Epi-1α,25-Dihydroxy-2β-(3-Hydroxypropoxy)Vitamin D3: 20-Epi-ED-71]. Proc. 14th Vitam. Workshop 2010, 121 (1), 25-28. https://doi.org/10.1016/j.jsbmb.2010.03.041.
(19A) Dussault, P. H.; Lee, H.-J.; Liu, X. Selectivity in Lewis Acid-Mediated Fragmentations of Peroxides and Ozonides: Application to the Synthesis of Alkenes, Homoallyl Ethers, and 1,2-Dioxolanes. J. Chem. Soc. Perkin 1 2000, No. 17, 3006-3013. https://doi.org/10.1039/B0013911.

### Example 2: Assessment of Efficacy

### 2.1 General Methods

Plasmodium falciparum Culture

P. falciparum parasites were maintained under routine culture conditions in red blood cells and RPMI 1640-Hepes with Glutamax, supplemented with 10 mM D-glucose, 480 μM hypoxanthine, 20 μg/mL gentamicin, 0.375% (w/v) ALBUMAX II, and 2.5% v/v heat-inactivated human serum [Maier and Rug, 2013]. Cultures were maintained at 37° C. under microaerophilic conditions (94% N₂, 5% CO₂, 1% O₂). All experiments used 307 wildtype parasites except where otherwise specified. Cultures were synchronised with 5% w/v D-sorbitol (Lambros et al., 1979).

### Human Foreskin Fibroblast (HFF) Culture

HFF cells were cultured under routine culture conditions in Dulbecco's Modified Eagle's Medium (DMEM) supplemented with 10% v/v NCS and anti-biotics at 37° C. in a humidified 5% CO₂ incubator.

Toxoplasma gondii Culture

Type 1 Toxoplasma gondii parasites expressing the fluorescent protein tdTomato were cultured under routine culture conditions in HFF cells and Dulbecco's Modified Eagle's medium (DMEM) supplemented with 1% (v/v) fetal bovine serum (FBS) and anti-biotics.

### Fluorescence Microscopy

Images were collected and deconvoluted on a Deltavision Deconvolution microscope at 1000× magnification, with a resolution of 0.067 μm per pixel. NBD and BODIPY fluorescence were detected at 475/28 nm excitation and 525/48 nm emission. Hoechst fluorescence (nucleic acid) was detected at 390/18 nm and ex/435/48 nm em. Resorufin fluorescence was detected at 575/25 nm ex and 626/45 nm em.

Within each experiment, images were collected under the same exposure conditions (without binning) and converted to TIFF files under the same brightness and contrast settings. Individual cells were cropped from larger images with Fiji ImageJ. No other manipulations were performed.

### Flow Cytometry

For quantification, events were measured on a LSR II or LSRFortessa Flow Cytometer. NBD/BODIPY fluorescence was detected at 488 nm ex/530 nm em. Hoechst 33342 (Thermo Fisher) fluorescence was detected at 410 nm ex/450 nm em. Resorufin fluorescence was detected at 561 nm ex/610 nm em. Mitotracker Deep Red was detected at 633 nm ex/670 nm em. Dehydroergosterol was detected at 355 nm ex/450 nm em.

Data were initially processed using FlowJo. RBCs were gated on FSC and SCC. iRBCs (positive) and uRBCs (negative) were differentiated by Hoechst or Mitotracker fluorescence. The geometric mean of fluorescence intensity (mean fluorescence intensity, MFI) and/or the percentage of each population (e.g. Hoechst positive) was calculated with FlowJo. Background fluorescence was subtracted based on unstained controls (buffer or solvent only). Except where otherwise specified, MFI data was normalised between experiments by setting the fluorescence of untreated uRBCs to 1.

### Statistics:

Except where otherwise specified, data were analysed in Prism 9 using one-way or two-way ANOVA with corrections for the false discovery rate using the two-stage setup method of Benjamini et a. (2006).

### Uptake of Fluorescent Cholesterol Analogues:

Cholesterol depleted media was prepared by removing lipoproteins by ultracentrifugation according to methods by Havel, Eder, and Bragdon (1955) with modification according to Renaud et al. (1982), Martin and van Golen (2012), and Foxx (2014) as described below.

The density of heat-inactivated human serum was adjusted to 1.21 g/mL by addition of KBr {M&B Pronalys JN351}. The solution was centrifuged at 220,000 g for 48 hours at 10° C. using a Beckman SW 41 Ti rotor. This top layer were carefully removed, and the bottom layer was then diluted with MilliQ water to wash and centrifuged for an additional 24 hours under the same conditions as above. The top layers were carefully removed as above. The bottom fraction was taken as cholesterol-depleted serum and transferred to 3.5 MWCO Dialysis tubing {Thermofisher 88242}. This was exhaustively dialysed in four changes of PBS (2 L, pH 7.4) over 24 hours at 4° C. The final product was sterilised by passing through a 0.2 μm filter and stored at −20° C. until use. Culture medium for this experiment was prepared from RPMI 1640-Hepes with Glutamax supplemented with 10 mM D-Glucose, 480 μM hypoxanthine, 20 μg/mL Gentamicin and 10% v/v cholesterol-depleted serum as described above (cholesterol-depleted media). Quantification of total cholesterol, free cholesterol, and cholesteryl ester was assessed using a Amplex™ Red Cholesterol Assay Kit {Thermofisher A12216} according to the manufacturer's instructions.

Ring-stage parasites at 4% parasitaemia and 2% haematocrit were incubated with 2.02 uM cholesterol analogues or solvent controls in cholesterol-depleted media for 24 hours at 37° C. under microaerophilic conditions. Cells were washed twice in phosphate buffered saline with glucose (PBS-G), and then resuspended in 5 μg/mL Hoechst 33342 in PBS-G and incubated for 20 minutes at 37° C. Samples were read on a flow cytometer or imaged with fluorescence microscopy as described above.

### Growth Inhibition Assays:

### Plasmodium Growth Inhibition Assays:

Growth inhibition was investigated according to methods described by Smilkstein et al. (2004) with modifications by Spry et al. (2013). Compounds or solvent controls (DMSO, ethanol, or ethyl acetate) were serially diluted 2-fold across flat-bottom 96-well plates. Complete culture media without compound was used as a negative control. Chloroquine (100-200 nM) {Sigma C6628} was used as a positive control to determine background fluorescence. The parasitaemia of a culture containing predominantly ring stage parasites was counted by Giemsa smear. The parasitaemia was adjusted to 1% by addition of fresh uRBCs, and added to the plate at a final haematocrit of 1%, with 200 μL per well. The plates were incubated at 37° C. under humid microaerophilic conditions. After 72 hours (or the indicated timepoint), the plates were transferred to a −18° C. freezer to stop the assay and lyse cells. Plates were thawed and lysate was mixed 1:1 with SYBR Safe DNA Gel Stain {Invitrogen 33102}(final concentration 1/10000) diluted in lysis buffer (20 mM TRIS, 5 mM EDTA, 0.008% w/v Saponin, 0.08% w/v Triton-X 100, pH 7.5). Fluorescence was read at 490 nm excitation/520 nm emission on a FLUOstar Optima fluorescence plate reader {BMG Labtech}.

### Toxoplasma Growth Inhibition Assays:

Growth inhibition was investigated as described previously (e.g. Rajendran et al., 2017). Fluorescent Toxoplasma gondii parasites were inoculated into an optical bottom 96-well plate containing confluent human foreskin fibroblast (HFF) host cells at a density of 2000 parasites/well, with a serial dilution of each compound, or media/DMSO alone. Plates were measured daily with a FluoStar Optima fluorescence plate reader at 540 nm excitation/590 nm emission for seven days, and the background fluorescence from the time 0 reading was subtracted from all other measurements. The 72-hour timepoint was chosen for further analysis because parasite growth was in the mid-logarithmic stage.

### HFF Growth Inhibition Assays:

HFF cells were released from a flask using 0.25% trypsin with 0.2 g/L EDTA and washed with culture media. Cells were incubated with a serial dilution of each compound, or media/DMSO alone. The protein synthesis inhibitor cycloheximide (10 μg/mL) was used as a positive control to determine background fluorescence. Approximately 5000 cells were seeded in each well. Plates were incubated at 37° C. for 72 hours (until reaching confluency) and the media was removed. Plates were transferred to a −18° C. freezer. Plates were thawed and SYBR Safe DNA Gel Stain (Invitrogen 33102) (final concentration 1/10000) diluted in lysis buffer (20 mM TRIS, 5 mM EDTA, 0.008% w/v saponin, 0.08% w/v Triton-X 100. pH 7.5) was added. Fluorescence was read at 490 nm excitation/520 nm emission on a FLUOstar Optima fluorescence plate reader.

### 2.2 Data

Growth inhibition data were normalised by subtracting background fluorescence from a no-growth control (high drug concentration or time=0 measurement as specified), and expressed as a percentage of the growth in the negative control (media alone or solvent control) either at the indicated timepoint, or to the highest measurement reached during the assay. Data were fitted with a four parameter [inhibitor] vs response curve in Prism 9, and the concentration inhibiting 50% of growth (IC₅₀) was calculated by the model.

### 2.3 Results

### The Red Blood Cell Plasma Membrane is Depleted of Cholesterol as the Parasites Develops and Accumulates Cholesterol

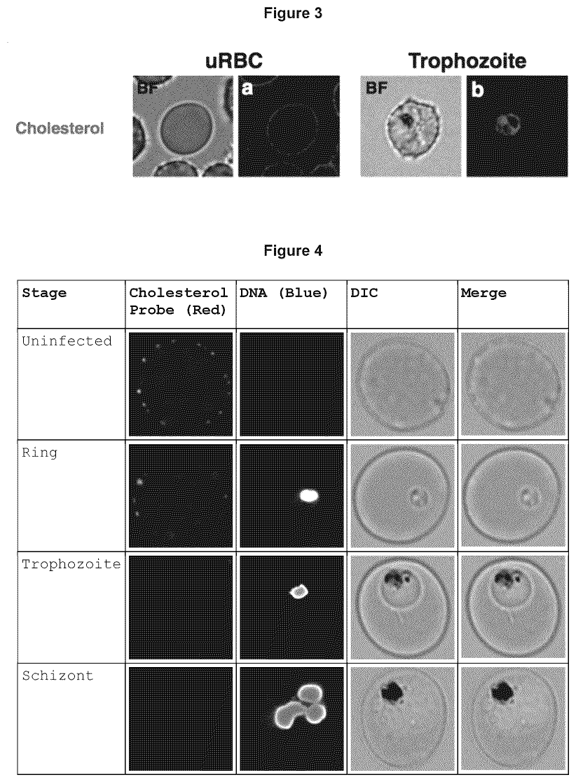

To assess the lipid pattern of blood stage P. falciparum, uninfected RBCs and RBCs infected with trophozoite P. falciparum were stained with 25-NBD cholesterol (a fluorescent cholesterol analogue), and the distribution of cholesterol observed by fluorescence imaging using a DeltaVision Elite microscope (Applied Precision) at the same fluorescent recording setting for the infected and uninfected RBCs. The results are shown in FIG. 3. As can be seen from FIG. 3, cholesterol was detected in the membrane of uninfected RBCs. However, fluorescence could be detected in the trophozoite in infected RBC, indicating that the parasite accumulates cholesterol.

To further assess the accumulation of cholesterol, uninfected RBC and P. falciparum parasites infected red blood cells were stained with a fluorescent probe, that binds to cholesterol (red) and DNA stain (blue). The results are shown in FIG. 4. As the parasite develops inside the red blood cell, less cholesterol can be detected on the red blood cell plasma membrane (note: probe does not cross membranes, hence only cholesterol exposed on the outside of the membrane is detected).

### Depletion of Cholesterol Inhibits Survival and Growth of Parasites

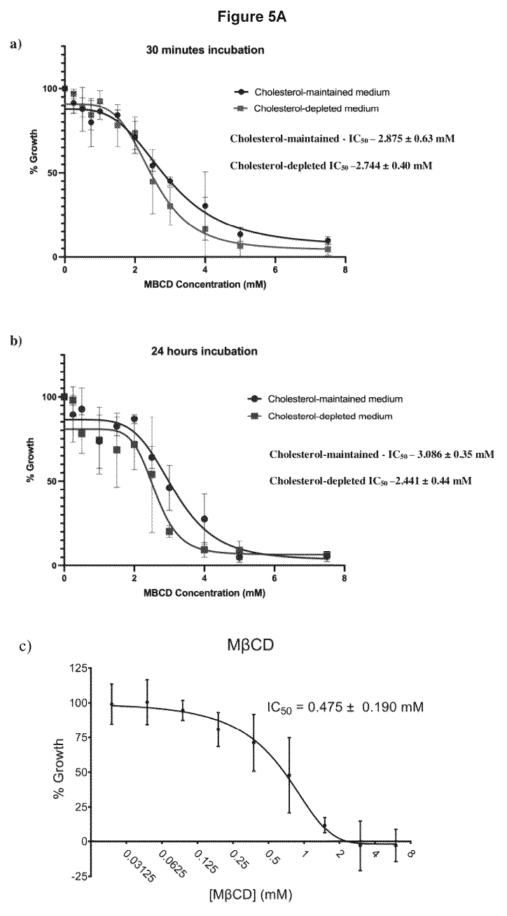

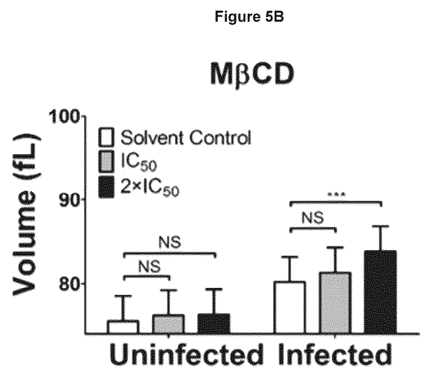

The effect of cholesterol depletion from RBC on P. falciparum proliferation over time was assessed by studying the concentration dependent effect of cholesterol depletion from RBCs on asexual growth of P. falciparum over 30 mins (a), 24 hrs (b) and 72 hrs (c), incubated in cholesterol-maintained and cholesterol-depleted conditions. The results are shown in FIG. 5A. FIG. 5A shows a reduction in parasite growth in RBC depleted of cholesterol compared to growth in RBC not depleted of cholesterol.

Depletion of cholesterol also leads to swelling of the infected host cell. Uninfected and infected red blood cells were incubated at different concentrations of MBCD, which depletes cholesterol from membranes. After 6 hours at the highest MBCD concentration (1.6 mM) the volume of infected RBCs increases significantly, whereas the same concentration has no effect on uninfected RBCs (FIG. 5B). This points towards an increased role of cholesterol in the infected RBCs.

Cholesterol Analogues with Modified Side Chains can be Taken Up by the RBC and Parasite

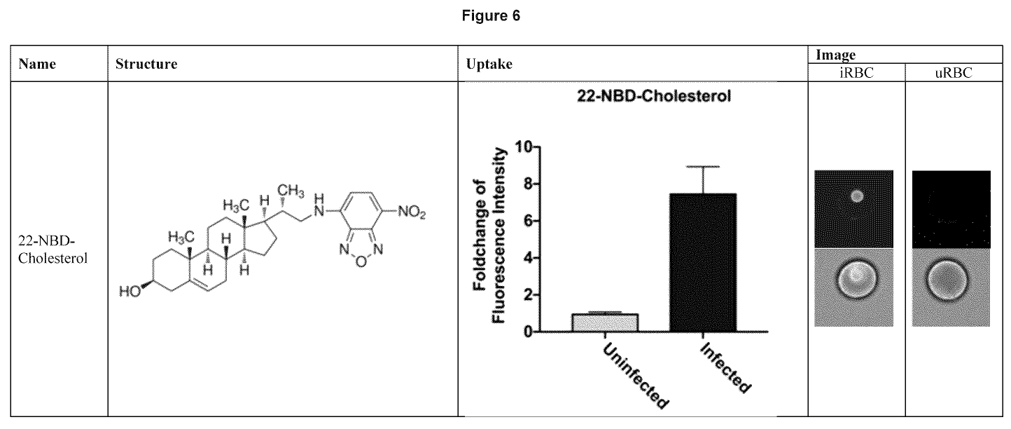

To determine if there was a difference in the rate of uptake of cholesterol in infected and uninfected RBCs, uninfected (uRBC) and infected (iRBC) red blood cells were incubated with the cholesterol analogue 22-NBD-cholesterol for 24 hrs. The results are shown in FIG. 6. Uptake in iRBC is increased ˜8-fold compared to uRBC. Accumulation of the cholesterol analogue in the parasite can be seen in infected RBCs.

Cholesterol Analogues with Modifications at the C17 are Taken Up More Efficiently than Analogues with C3 Modifications

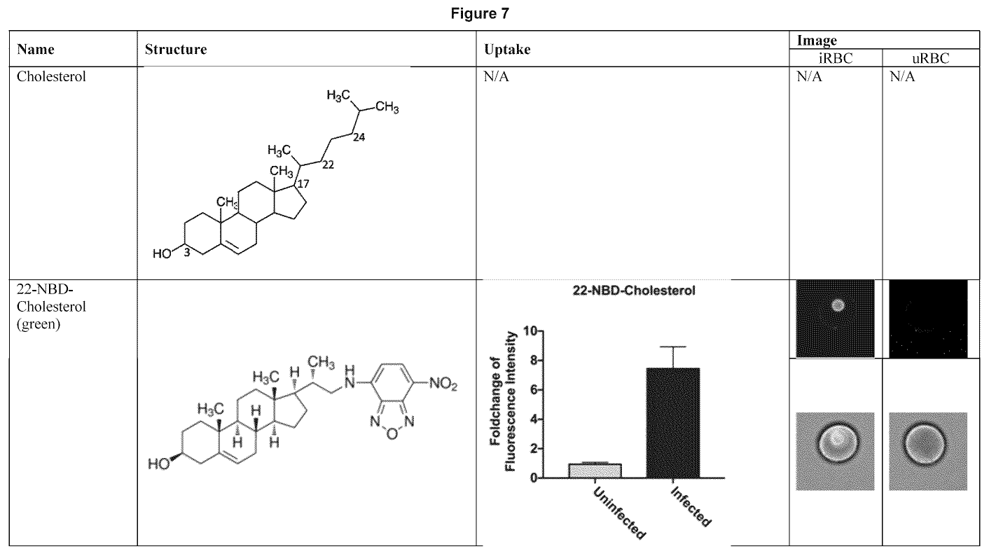

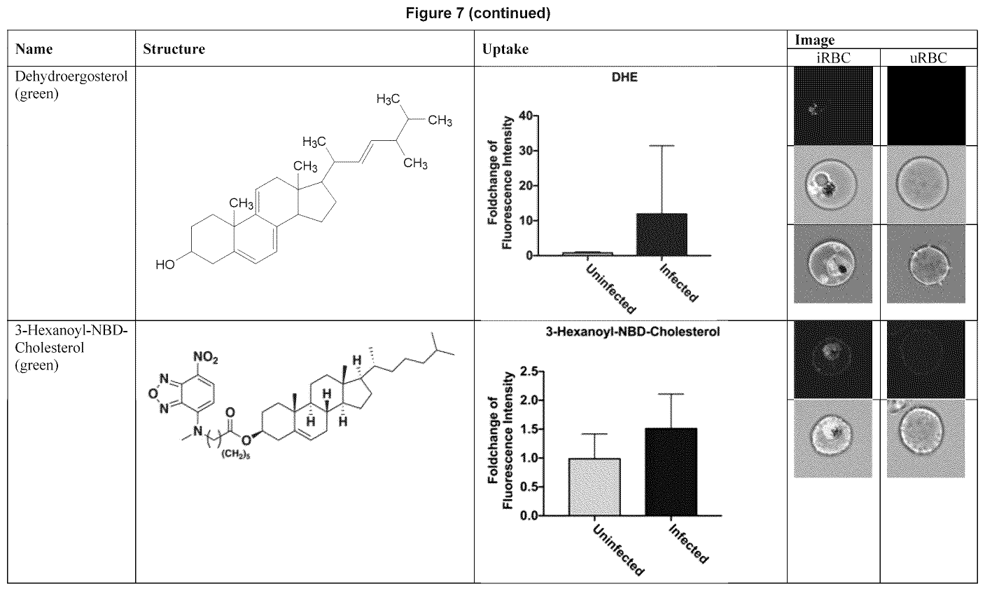

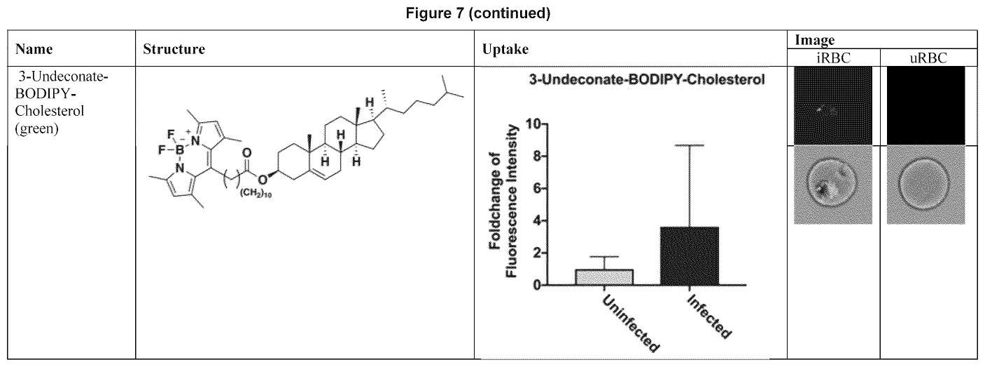

C17 and C3 of cholesterol was modified by the addition of different fluorophores (except dehydroergosterol, which exhibits intrinsic fluorescence) as shown in FIG. 7. The second column of FIG. 7 shows the structure of the compounds. Uptake into infected and uninfected red blood cells after 24 hrs was quantified for each compound by measuring changes in fluorescence (third column) and visualised using a Deltavision Deconvolution microscope (fourth/fifth column). Steroid conjugates/cholesterol analogues in green and parasite DNA in blue; except dehydroergosterol, where parasite mitochondria are also in red.

As can be seen from FIG. 7, compounds with modifications at or after C17 are taken up more efficiently into infected red blood cells compared to uninfected red blood cells (7-12-fold); whereas the uptake of compounds with C3 modifications increases only 1.5-3-fold. The exception to this pattern is 24-BODIPY-cholesterol, which seems to get stuck at the RBC membrane.

### Coupling of a Known Anti-Malarial Compound to Steroid Increases its Efficacy and Pharmacological Profile

Primaquine is an anti-malarial compound that is used for the treatment of malaria. It is one of the few drugs that efficiently inhibits liver stages. It also has very good gametocytocidal activity, but the activity against asexual blood stages of the parasite is moderate. However, the use of primaquine is contraindicated for patients with glucose-6-phosphate dehydrogenase deficiencies, since it can cause fatal haemolysis in these patients. In the human body the activity of primaquine is 1,000-fold enhanced since the P450 NADPH-oxidoreductase present in the liver and bone marrow generates H₂O₂ from the primaquine metabolites, which kill the parasites. The following experiments were done in the absence of P450 NADPH-oxidoreductase.

Primaquine was coupled to C17 of steroid as described above, and the resulting compound was compared to primaquine alone.

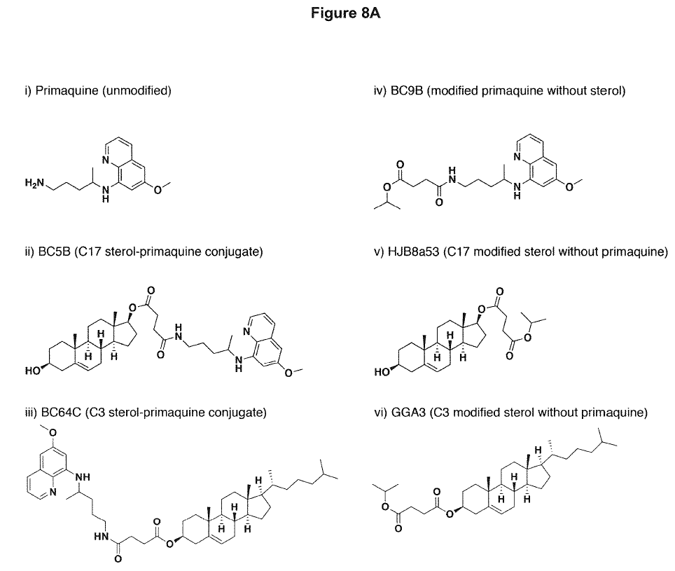

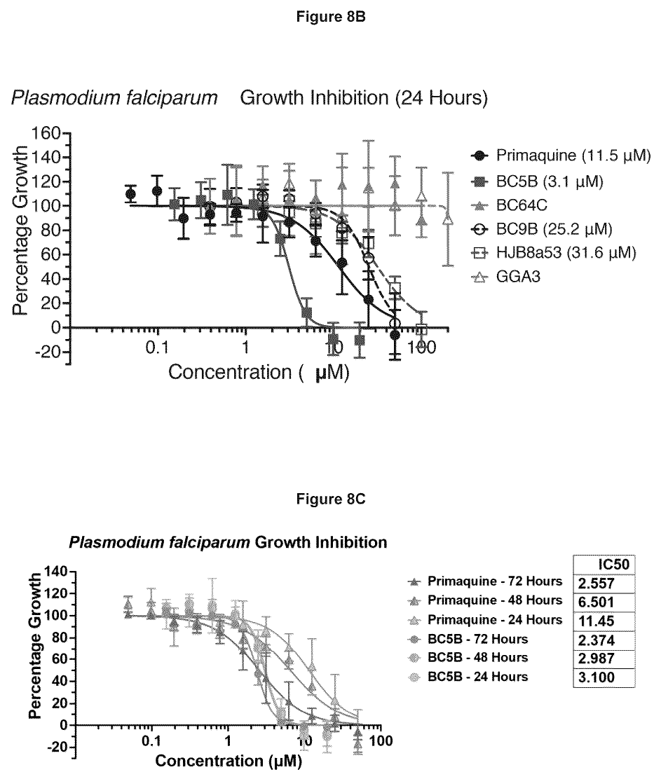

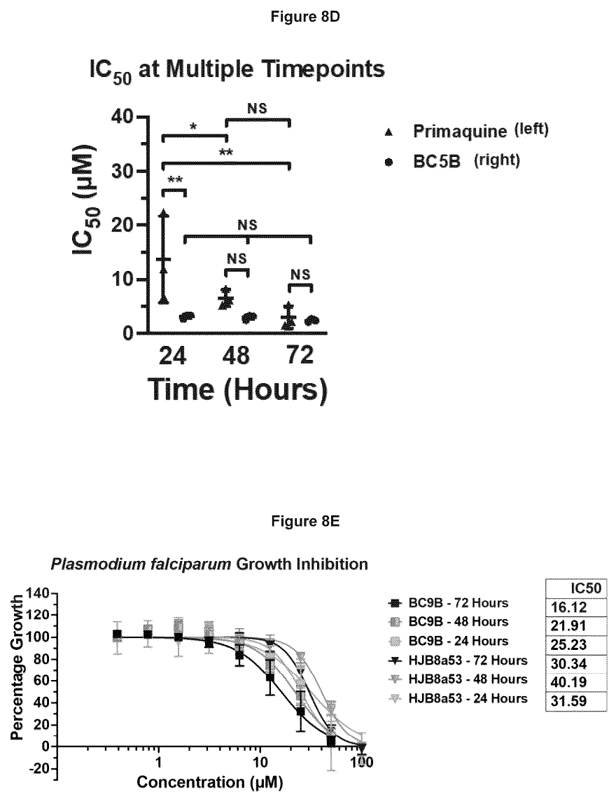

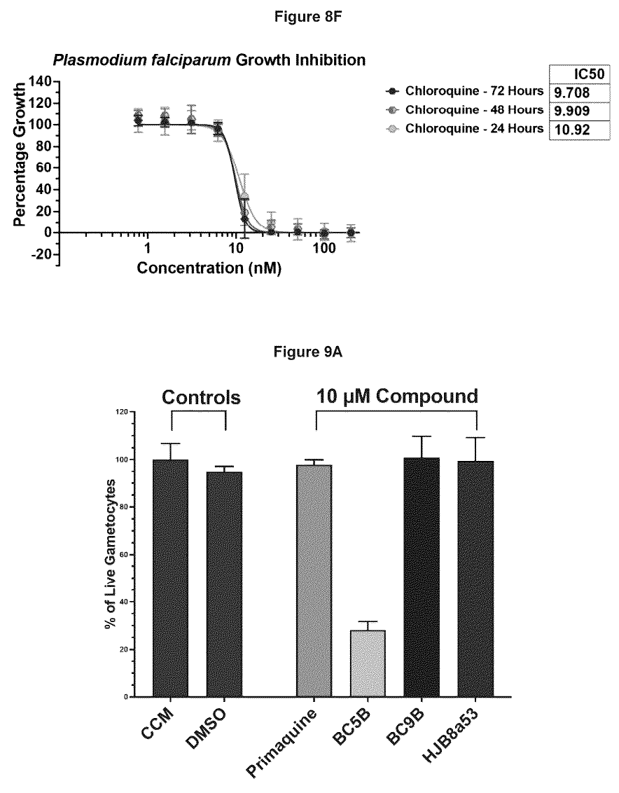

FIG. 8A shows the structure of primaquine alone, primaquine coupled to steroid (via a linker at C17) (BC5B), primaquine coupled to a linker (BC9B), a steroid coupled to a linker (at C17) (HJB8a53), a steroid coupled to a linker (at C13) (GGA3), and primaquine coupled to steroid (via a linker at C3) (BC64C).

FIG. 8B shows dose response curves of primaquine alone, primaquine coupled to steroid (via a linker), primaquine coupled to a linker, and steroid coupled to a linker, showing the effect of these compounds on asexual P. falciparum stages. Control compounds (primaquine & linker or steroid & linker) were used to show specificity.

As can been seen from FIG. 8B, BCSB (steroid-coupled primaquine) exhibits not only a lower IC₅₀ than primaquine alone, but the dose response curve is also steeper. Generally, a flatter curve indicates a potential higher risk of the emergence of drug-resistant parasites because parasites survive over a broader concentration window. The difference is also clear, when comparing IC10 values (primaquine alone 12 μM, primaquine-steroid 3 μM). Control compounds (primaquine & linker or steroid & linker) do not show the same enhanced activity than primaquine & steroid.

The IC50 values of steroid-coupled primaquine and primaquine were compared and are shown in FIGS. 8C and 8D. Comparing the IC50 values of primaquine and primaquine coupled to steroid at different time points reveals a decrease in IC50 of primaquine over this period of time. In comparison, the value for cholesterol-coupled primaquine stays fairly constant. This indicates a potentially faster mode of action of the coupled compound. In addition, coupling also alters the shape of the curve (increasing its steepness), which might also indicate a different dynamic of its mode of action. A narrower range in which the cell population consists of a mixture of dead and alive cells might help preventing the occurrence of drug resistance against this compound.

The IC50 values for steroid-coupled primaquine were also much lower than that for controls BC9B and HJB8a53 (primaquine & linker or cholesterol & linker) (FIG. 8E).

The IC50 values for steroid-coupled primaquine were also lower than that for commercially available anti-malarial chloroquine against asexual P. falciparum stages (FIG. 8F).

Steroid-Coupled Compounds Also Show Increase Efficacy Against Gametocyte and Hence could Act a Transmission Blocking Agent.

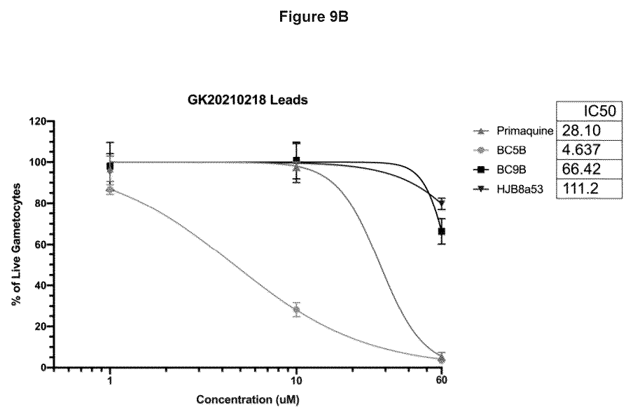

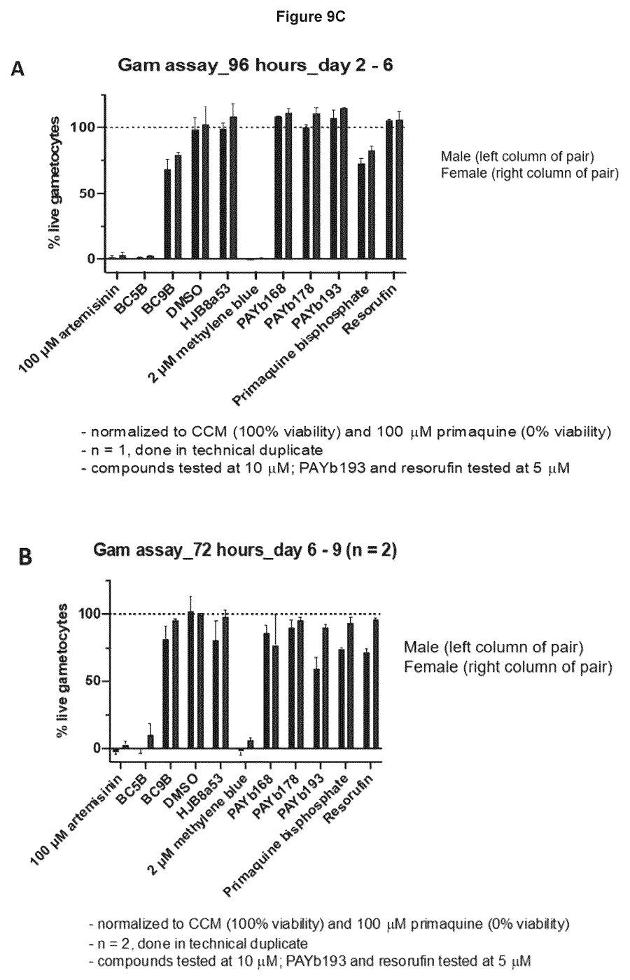

The effect of steroid-coupled primaquine on P. falciparum gametocytes over 72 hrs (stage III—IV) was assessed. The results are shown in FIGS. 9A and 9B. The results show that steroid-coupled primaquine was more effective at inhibiting growth of gametocytes compare to primaquine alone, or controls BC9B and HJB8a53 (primaquine & linker or steroid & linker).

The IC50 for steroid-coupled primaquine on P. falciparum gametocytes over 72 hrs (stage III—IV) was much lower that primaquine alone, or controls BC9B and HJB8a53 (FIG. 9B). Steroid-coupled primaquine seems to be 6-times more effective against late-stage gametocytes than primaquine alone. However, the curve becomes flatter; (n=1)—in technical duplicates.

The sex-specific effect of different compounds against early (I-III) stage (A) and late stage (III-IV) P. falciparum gametocytes was assessed and the results shown in FIG. 9C. The data indicates that both early (A) and late-stage (B) gametocytes are equally susceptible to the BC5B. Both gametocyte sexes can be targeted equally with the coupled compounds.

Steroid-Coupled Compounds can be Released from Steroid Once it is Taken Up by the Parasite

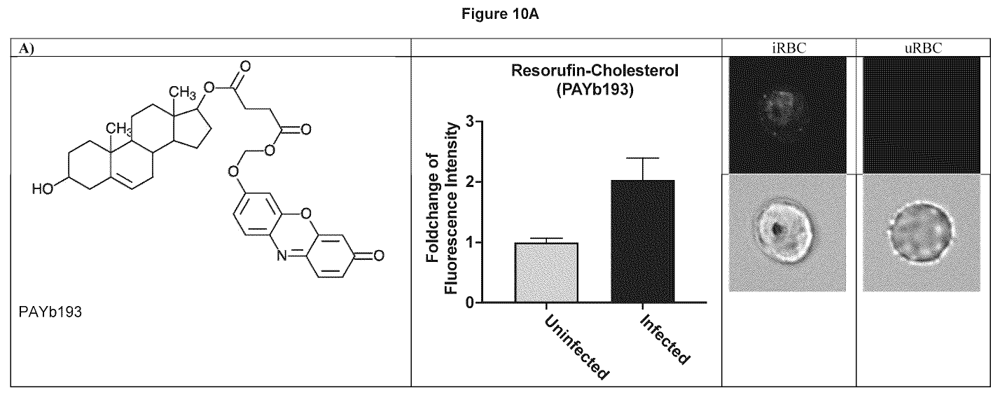

Resorufin was coupled to a steroid at C17 as described above. The structure of the conjugate (PAYb193) is shown in FIG. 10A and below.

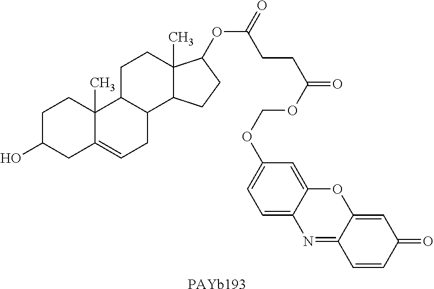 [SMILES: "PAYb193" ; CC12CCC(O)CC1=CCC1C2CCC2(C)C(OC(=O)CCC(=O)OCOc3ccc4nc5ccc(=O)cc-5oc4c3)CCC12]

The ability of the steroid-resorufin conjugate to inhibit growth of P. falciparum was assessed.

FIG. 10A shows that the cholesterol-resorufin conjugate is cleaved within the cell, indicating that the probe is taken up and metabolised. Uptake of the probe is approximately 2-fold higher in iRBCs than uRBCs. Resorufin fluorescence (red) is observed within the parasite (DNA stained blue). FIG. 10B is a dose response curve showing that the cholesterol-resorufin probe can kill the parasite at lower concentrations than the uncoupled resorufin base molecule, potentially indicating that the higher uptake into iRBCs can be employed as a specific drug delivery system.

Steroid-Coupled Compounds Also Show Increased Efficacy Against Toxoplasma Gondii—Another Apicomplexan Parasite that Causes Disease in Humans

Toxoplasma gondii is less susceptible to primaquine than Plasmodium (Holfels et al, 1994) and hence primaquine is not used for the treatment of toxoplasmosis. It is unclear whether this difference is due to insufficient uptake of primaquine or whether the target molecule is missing in Toxoplasma.

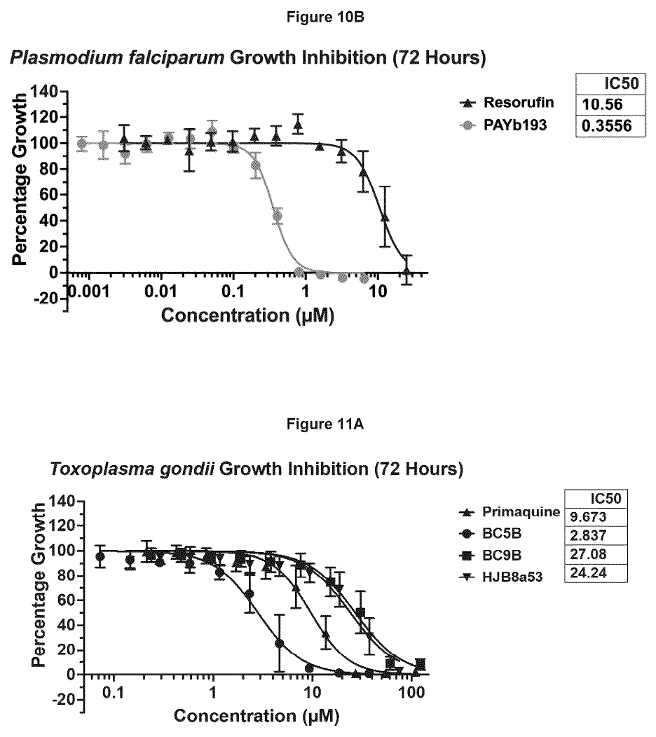

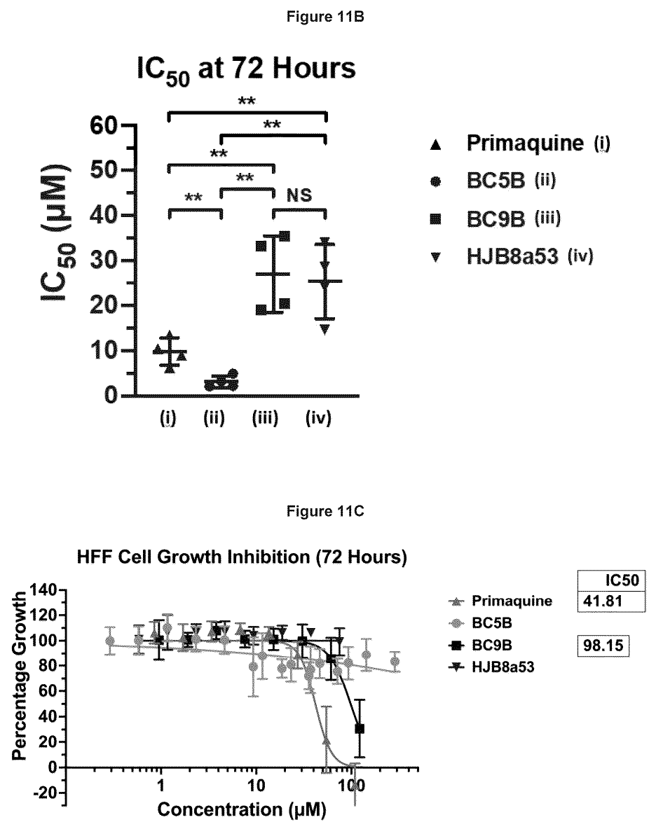

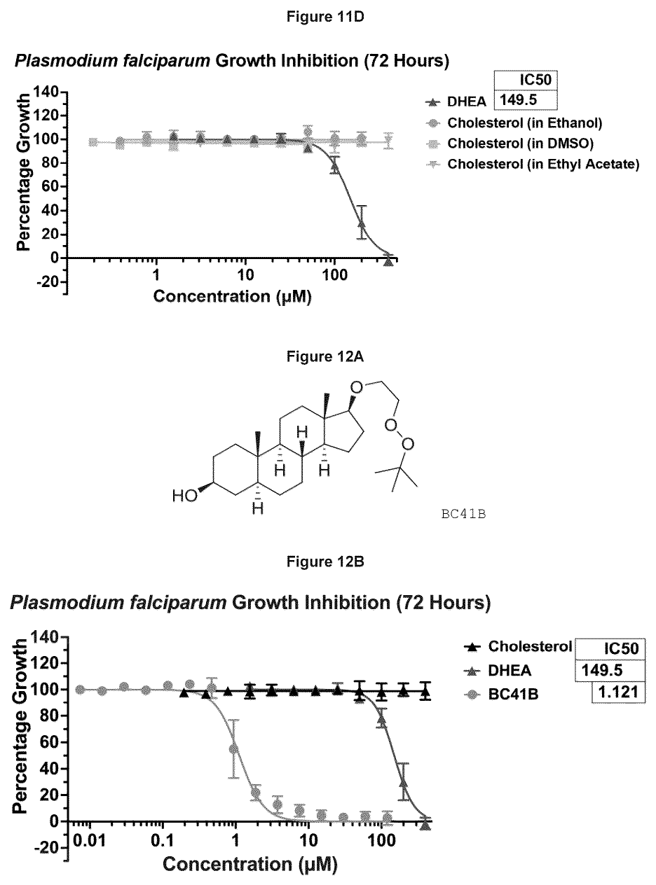

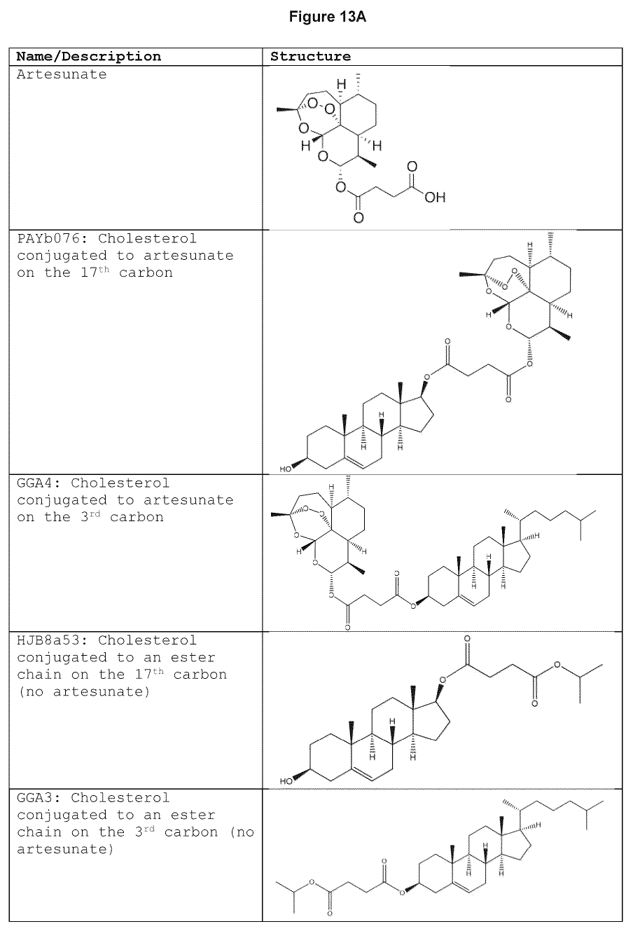

The ability of the steroid-primaquine conjugate to inhibit Toxoplasma gondii growth was assessed over 72 hours. The results are shown in FIGS. 11A and 11B. The IC₅₀ values of steroid-coupled primaquine (BC5B), primaquine, primaquine & linker (BC9B), steroid & linker (HJB8a53) were determined via a dose-response curve (FIGS. 11A and 11B). Cholesterol-primaquine conjugates inhibit the growth of Toxoplasma gondii with higher efficacy than primaquine, indicating that steroid-coupling can enhance the effectiveness of drugs in other Apicomplexan parasites. In addition, other (non-apicomplexan) parasites of veterinary importance also rely on the uptake of host cholesterol and other steroids and the delivery system will also relevant to these parasites.

Steroid-coupled primaquine displays increased safety profile against the host cells of Toxoplasma gondii

To investigate selectivity and safety of the compounds, the inhibitory effect of steroid-coupled primaquine on the growth of human foreskin fibroblasts (HFF)—the host cells of Toxoplasma gondii—was assessed over 72 hrs. The IC₅₀ values of steroid-coupled primaquine (BC5B), primaquine, primaquine & linker (BC9B), steroid & linker (HJB8a53) were determined via a dose-response curve. The results are shown in FIG. 11C.

It was found that BC5B was not able to completely inhibit more than 50% of the growth of HFF cells at the range of concentrations tested, and thus no IC₅₀ could be reliably calculated. This likely indicates that the observed T. gondii inhibition is not due to host cell death. Together, these data indicate that compared to unconjugated primaquine, BC5B is more selective for parasite inhibition, and less toxic to human cells.

### Steroid by Itself and in Different Solvents is not Toxic to the Parasite

To exclude the possibility that steroid by itself or the different solvents being used have an effect on the proliferation of asexual stages of P. falciparum, the IC50 of DHEA, cholesterol in ethanol, cholesterol in DMSO and cholesterol in ethyl acetate, was determined (FIG. 11D). No inhibitory effect could be detected in the concentration range being used. Dehydroepiandrosterone (DHEA) influences proliferation, although at relatively high concentrations.

Steroid can not Only be Used for Drug-Delivery, but Modifying Steroid can Turn it into a Drug-Like Molecule with Anti-Malarial Activity

Since the presumed inhibitory action of primaquine is the generation of free radicals in the cell, steroid was conjugated to a peroxide (BC41B) and tested for its effect against asexual stages of Plasmodium falciparum over 72 hours. The structure of the compound is shown in FIG. 12A and below, and the results achieved are shown in FIG. 12B.

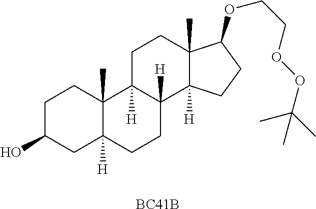

The IC₅₀ of steroid-bound peroxide is 1 μM, whereas primaquine alone displays an IC₅₀ of 3.2 μM (see 7). Hence, modified cholesterol has the potential to be used as a drug itself (e.g. when conjugated to a peroxide).

### Conjugation of Other Compounds

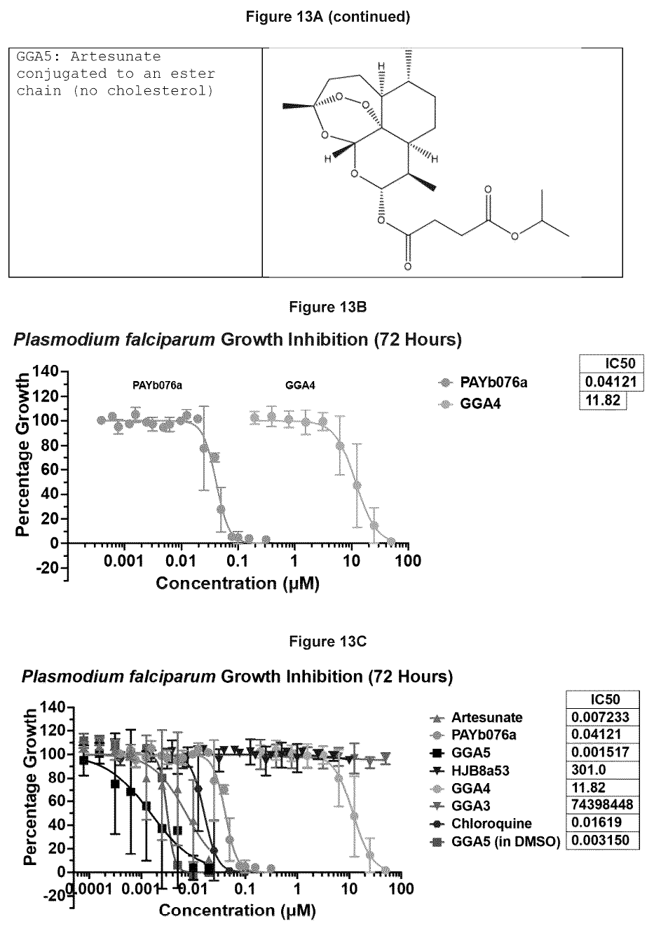

The anti-malarial drug artesunate was selected for preliminary studies. Two different conjugation sites were selected: the fatty acid side chain at C17 (similar in structure to Resorufin-Cholesterol), and the 3-hydroxyl group (similar in structure to 3-hexanoyl-NBD-Cholesterol). The drug was linked to the steroid with a short ester chain. Structures of the compounds are shown in FIG. 13 A and below. Compounds HJB8a53, GGA3 and GGA5 were synthesised as controls: steroid conjugated to only the ester chain on C17 or C3 without artesunate, and artesunate conjugated to only the ester chain without cholesterol.

| Name/Description | Structure |
|---|---|
| Artesunate | 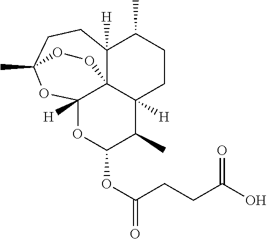 [SMILES: C[C@H]1[C@H](OC(=O)CCC(=O)O)O[C@@H]2O[C@]3(C)CC[C@H]4[C@H](C)CC[C@@H]1[C@@]24OO3] |
| PAYb076: Cholesterol conjugated to artesunate on the 17^{th} carbon (C-17-art) | 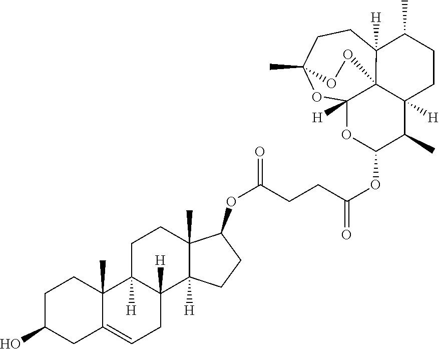 |
| GGA4: Cholesterol conjugated to artesunate on the 3^{rd} carbon | 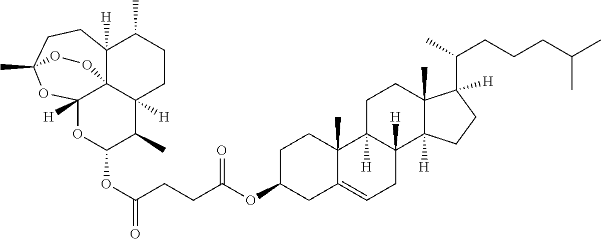 |
| HJB8a53: Cholesterol conjugated to an ester chain on the 17^{th} carbon (no artesunate) (C-17-link) | 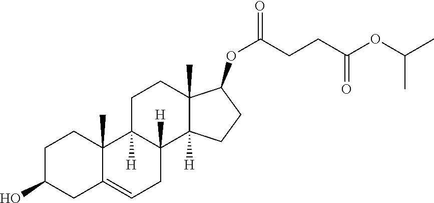 |
| GGA3: Cholesterol conjugated to an ester chain on the 3^{rd} carbon (no artesunate) (C-3-link) | 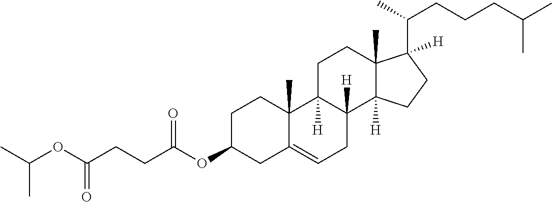 |
| GGA5: Artesunate conjugated to an ester chain (no cholesterol) (Art- link) | 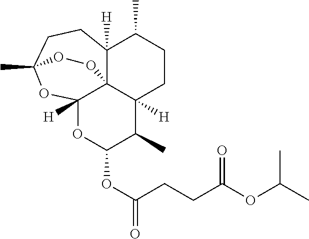 [SMILES: CC(C)OC(=O)CCC(=O)O[C@@H]1O[C@@H]2O[C@]3(C)CC[C@H]4[C@H](C)CC[C@@H]([C@H]1C)[C@@]24OO3] |

The dose-response effect of differently coupled steroid-artesunate conjugates on the proliferation of asexual P. falciparum parasites was assessed over 72 hrs. The results are shown in FIG. 13B.

The results indicate a clear difference in IC₅₀ depending on which side of the steroid molecule the drug was conjugated to, with the C17 coupling showing a more than 250-fold lower IC₅₀. This is consistent with the fluorescent cholesterol analogue uptake data above, where 22-NBD-Cholesterol demonstrated a much higher difference in uptake between uRBCs and iRBCs compared to that of 3-hexanoyl-NBD-Cholesterol (see section 5 & 6).

The dose-response effects of differently coupled steroid-artesunate conjugates on the proliferation of asexual P. falciparum parasites over 72 hrs was assessed. The results are shown in FIG. 13C. Both of the steroid-artesunate conjugates inhibited the growth of P. falciparum in a concentration dependent manner, though the IC₅₀ was higher than the starting artesunate molecule. Notably, artesunate is already a highly effective inhibitor of P. falciparum growth, and has already been through an extensive optimisation process—it is possible that the changes interfere with the existing uptake pathway of the drug.

These results demonstrate the potential of exploiting increased cholesterol uptake into iRBCs as a method of delivering drugs directly to the parasite.

The following references relate to the description of Example 2 above.

### REFERENCES

(1) Fulmer, G. R.; Miller, A. J. M.; Sherden, N. H.; Gottlieb, H. E.; Nudelman, A.; Stoltz, B. M.; Bercaw, J. E.; Goldberg, K. I. NMR Chemical Shifts of Trace Impurities: Common Laboratory Solvents, Organics. and Gases in Deuterated Solvents Relevant to the Organometallic Chemist. Organometallics 2010, 29 (9), 2176-2179. https://doi.org/10.1021/om100106e.
(2) Yamauchi, T.; Kato, M.; Mikami, T.; Fujimura, Y. Synthesis of 1-Deoxymaxacalcitol. Heterocycles 2005, 65 (9), 2111.
(3) Moreira, V. M. A.; Vasaitis, T. S.; Guo, Z.; Njar, V. C. O.; Salvador. J. A. R. Synthesis of Novel C17 Steroidal Carbamates: Studies on CYP17 Action, Androgen Receptor Binding and Function, and Prostate Cancer Cell Growth. Steroids 2008, 73 (12), 1217-1227. https://doi.org/10.1016/j.steroids.2008.05.010.
(4) András Keglevich; Vivian Zsiros; Péter Keglevich*; Áron Szigetvári; Miklós Dékány; Csaba Szántay; Erzsébet Memyák; János Wölfling and László Hazai*. Synthesis and In Vitro Antitumor Effect of New Vindoline-Steroid Hybrids. Curr. Org. Chem. 2019, 23 (8), 959-967. https://doi.org/10.2174/1385272823666190614113218.
(5) Fernandes, I.; Vale. N.; de Freitas, V.; Moreira, R.; Mateus, N.; Gomes, P. Anti-Tumoral Activity of Imidazoquines, a New Class of Antimalarials Derived from Primaquine. Bioorg. Med. Chem. Lett. 2009, 19 (24), 6914-6917. https://doi.org/10.1016/j.bmcl.2009.10.081.
(6) Yamashita, T.; Kawai, N.; Tokuyama, H.; Fukuyama, T. Stereocontrolled Total Synthesis of (−)-Eudistomin C. J. Am. Chem. Soc. 2005, 127 (43), 15038-15039. https://doi.org/10.1021/ja055832h.
(7) Krausova, B.; Slavikova, B.; Nekardova, M.; Hubalkova. P.; Vyklicky, V.; Chodounska, H.; Vyklicky, L.; Kudova, E. Positive Modulators of the N-Methyl-d-Aspartate Receptor: Structure-Activity Relationship Study of Steroidal 3-Hemiesters. J. Med. Chem. 2018, 61 (10), 4505-4516. https://doi.org/10.1021/acs.jmedchem.8b00255.
(8) Liu, D.; Stuhmiller, L. M.; McMorris, T. C. Synthesis of 15β-Hydroxy-24-Oxocholesterol and 150,29-Dihydroxy-7-Oxofucosterol. J. Chem. Soc. Perkin 11988, No. 8, 2161-2167. https://doi.org/10.1039/P19880002161.
(9) Calogeropoulou, T.; Avionitis, N.; Minas, V.; Alexi, X.; Pantzou, A.; Charalampopoulos, I.; Zervou, M.; Vergou, V.; Katsanou, E. S.; Lazaridis, I.; Alexis, M. N.; Gravanis, A. Novel Dehydroepiandrosterone Derivatives with Antiapoptotic, Neuroprotective Activity. J. Med. Chem. 2009, 52 (21), 6569-6587. https://doi.org/10.1021/jm900468p.
(10) Shen, Y.; Burgoyne, D. L. Efficient Synthesis of IPL576,092: A Novel Anti-Asthma Agent. J. Org. Chem. 2002, 67 (11), 3908-3910. https://doi.org/10.1021/jo0108717.
(11) Upsani, R. B.; Fick, D. B.; Hogenkamp, D. J.; Lan, N. C. Neuroactive Steroids of the Androstane and Pregnane Series. U.S. Pat. No. 5,925,630A, Jul. 20, 1999.
(12) Epp, J. B.; Widlanski, T. S. Facile Preparation of Nucleoside-5′-Carboxylic Acids. J. Org. Chem. 1999, 64 (1). 293-295. https://doi.org/10.1021/jo981316g.
(13) Kratena, N.; Biedermann, N.; Stojanovic, B.; Göschl, L.; Weil, M.; Enev, V. S.; Gmeiner, G.; Gärtner, P. Synthesis of a Human Long-Term Oxymetholone Metabolite. Steroids 2019, 150, 108430. https://doi.org/10.1016/j.steroids.2019.108430.
(14) Hosoda, H.; Fukushima, D. K.; Fishman. J. Convenient, High Yield Conversion of Androst-5-Ene-3.Beta., 17.Beta.-Diol to Dehydroisoandrosterone. J. Org. Chem. 1973, 38 (24), 4209-4211. https://doi.org/10.1021/jo00963a028.
(15) Felzmann, W.; Gmeiner, G.; Gärtner, P. First Synthesis of a Pentadeuterated 3-Hydroxystanozolol—an Internal Standard in Doping Analysis. Steroids 2005, 70 (2). 103-110. https://doi.org/10.1016/j.steroids.2004.10.002.
(16) Kiobucki, M.; Grudniewska, A.; Smuga, D. A.; Smuga, M.; Jarosz, J.; Wietrzyk, J.; Maciejewska, G.; Wawrzeńczyk, C. Syntheses and Antiproliferative Activities of Novel Phosphatidylcholines Containing Dehydroepiandrosterone Moieties. Steroids 2017, 118, 109-118. https://doi.org/10.1016/j.steroids.2016.12.015.
(17) Jao, S.-C.; Chen, J.; Yang, K.; Li. W.-S. Design of Potent Inhibitors for Schistosoma japonica Glutathione S-Transferase. Bioorg. Med. Chem. 2006, 14 (2), 304-318. https://doi.org/10.1016/j.bmc.2005.07.077.
(18) Hatakeyama, S.; Yoshino, M.; Eto, K.; Takahashi, K.; Ishihara, J.; Ono, Y.; Saito, H.; Kubodera, N. Synthesis and Preliminary Biological Evaluation of 20-Epi-Eldecalcitol [20-Epi-1α,25-Dihydroxy-2β-(3-Hydroxypropoxy)Vitamin D3: 20-Epi-ED-71]. Proc. 14th Vitam. Workshop 2010, 121 (1), 25-28. https://doi.org/10.1016/j.jsbmb.2010.03.041.
(19) Dussault, P. H.; Lee, H.-J.; Liu, X. Selectivity in Lewis Acid-Mediated Fragmentations of Peroxides and Ozonides: Application to the Synthesis of Alkenes, Homoallyl Ethers, and 1,2-Dioxolanes. J. Chem. Soc. Perkin 12000, No. 17, 3006-3013. https://doi.org/10.1039/B001391I.
Benjamini Y, Kreiger A M, Yekutieli D. Adaptive linear step-up procedures that control the false discovery rate. Biometrika. 2006; 93(3):491-507.
Foxx K K. Depleting Lipoproteins from Serum. 2014; Available at: https://cdn.shopify.com/s/files/i/1348/9429/files/LipoproteinDepletedSerumMethod.pdf (Accessed: 17 Oct. 2017).
Havel R J, Eder H A, Bragdon J H. The distribution and chemical composition of ultracentrifugally separated lipoproteins in human serum. J Clin Invest. 1955 Sep. 1; 34(9):1345-53.
Lambros C, Vanderberg J P. Synchronization of Plasmodium falciparum erythrocytic stages in culture. J Parasitol. 1979 June; 65(3):418-20.
Maier A, Rug M. In vitro culturing Plasmodium falciparum erythrocyte stages. In: Menard R, editor. Malaria: Methods and Protocols. USA: Humana Press; 2013. p. 3-15.
Maier A G, Matuschewski K, Zhang M, Rug M. Plasmodium falciparum. Trends Parasitol. 2019 Dec. 27:35(6):481-2.
Martin B., van Golen K. A comparison of cholesterol uptake and storage in inflammatory and noninflammatory breast cancer cells. Int J Breast Cancer. 2012 Dec. 31;
Rajendran E, Hapuarachchi S V., Miller C M, Fairweather S J, Cai Y, Smith N C, et al. Cationic amino acid transporters play key roles in the survival and transmission of apicomplexan parasites. Nat Commun. 2017 Feb. 16; 8.
Renaud J F, Scanut A M, Kazazoglou T, Lombet A, Romey G, Lazdunski A M. Normal serum and lipoprotein-deficient serum give different expressions of excitability, corresponding to different stages of differentiation, in chicken cardiac cells in culture. Cell Biol. 1982:79:7768-72.
Smilkstein M, Sriwilaijaroen N, Kelly J X, Wilairat P, Riscoe M. Simple and inexpensive fluorescence-based technique for high-throughput antimalarial drug screening. Antimicrob Agents Chemother. 2004 May 1; 48(5):1803-6.
Spry C, Macuamule C, Lin Z, Virga K G, Lee R E, Strauss E, et al. Pantothenamides Are Potent, On-Target Inhibitors of Plasmodium falciparum Growth When Serum Pantetheinase Is Inactivated. PLoS One. 2013 Feb. 6; 8 (2):e54974.
Tran P N, Brown S H J, Rug M, Ridgway M C, Mitchell T W, Maier A G. Changes in lipid composition during sexual development of the malaria parasite Plasmodium falciparum. Malar J. 2016 Dec. 6; 15(1):73.

### Example 3: Assessment of Efficacy

Parasites such as the malaria parasite P. falciparum are critically dependent on nutrients from their host to survive and multiply inside host cells. P. falciparum parasites take up organic and inorganic substances from both the host cell and the extracellular environment to fuel their development and multiplication. Interference with these processes can lead to parasite death and therefore be the basis of successful drug strategies. P. falciparum parasites cannot synthesise cholesterol, and instead source this lipid from the host, depleting the host cell membrane.

The inventors therefore investigated the uptake and inhibitory effects of conjugate compounds, where proven antimalarial drugs, primaquine and artesunate, were attached to steroids which mimic the structure of cholesterol. The inventors found that fluorescent cholesterol analogues were delivered from the extracellular environment to the intracellular parasite, and that conjugating antimalarial drugs could improve the inhibitory effects of these drugs against multiple parasite lifecycle stages, multiple parasite species, and drug-resistant parasites, whilst also lowering the toxicity to human host cells. The inventors also showed that steroids with introduced peroxides can act as a drug-like molecule with antimalarial activity. These results provide evidence that cholesterol mimics can act as a drug delivery system against Apicomplexan parasites which could defeat drug resistance.

Cholesterol is a steroidal nutrient which serves as a vital membrane component of animal cells and some protozoa. Cholesterol consists of a tetracyclic hydrocarbon ring system, two methyl groups, a saturated hydrocarbon side chain from C-17, and a hydroxy group from C-3, as shown below (1A).

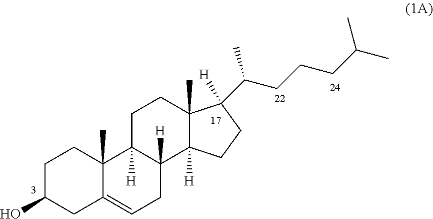

Neither parasite nor its host red blood cell (RBC) can synthesise cholesterol de novo, but cholesterol is vital to parasite survival. Despite this lack of synthesis capacity, cholesterol accumulates in infected RBCs (iRBCs), particularly in late stage (trophozoite and schizont) iRBCs and gametocytes. Cholesterol storage in gametocytes might be important for transmission, where parasites encounter a low cholesterol environment in the mosquito. Cholesterol normally localises to the RBC membrane, but upon Plasmodium infection, cholesterol is depleted from the RBC membrane and accumulates in the intracellular parasite, and interrupting this process leads to altered cholesterol distribution and parasite death. Furthermore, depletion of cholesterol from the RBC membrane using methyl-β-cyclodextrin can stop parasite development and prevent its invasion. Clinical data indicate that lipoprotein-bound blood cholesterol is lower in malaria patients, potentially indicating that Plasmodium parasites act as a cholesterol sink. Together, these observations indicate that cholesterol moves from the RBC membrane and the extracellular environment into the parasite.

The inventors have shown that conjugating cholesterol to an anti-malarial drug can hijack existing cholesterol uptake pathways to maximize drug delivery to the parasite, potentially increasing efficacy.

### Results

Parasitised RBCs Take Up Fluorescent Cholesterol Analogues, Particularly Those with Side Chain Modifications

To gain insights into how cholesterol gets into the parasites, the inventors tested several commercially available fluorescent cholesterol analogues for accumulation in intra-erythrocytic parasites. The inventors started with the naturally occurring fungal sterol dehydroergosterol (1B):

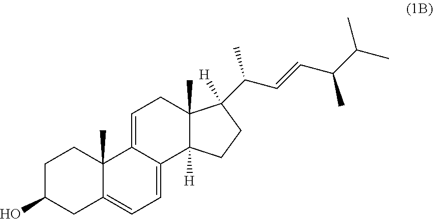 [SMILES: CC(C)[C@@H](C)/C=C/[C@@H](C)[C@H]1CC[C@H]2C3=CC=C4C[C@@H](O)CC[C@]4(C)C3=CC[C@]12C]

Dehydroergosterol is structurally similar to and mimics the properties of cholesterol, but exhibits intrinsic fluorescence due to the presence of additional double bonds. We incubated RBC infected with ring stage parasites with dehydroergosterol for 24 hours, and imaged the resulting trophozoite infected RBCs using deconvolution fluorescence microscopy. Fluorescence was visible in iRBCs, with the fluorescence concentrated around the parasite, indicating uptake of the compound.

The inventors next investigated the uptake of analogues where a fluorophore was conjugated from either the C-3, C-22, or C-24 position of cholesterol (IC—IF below).

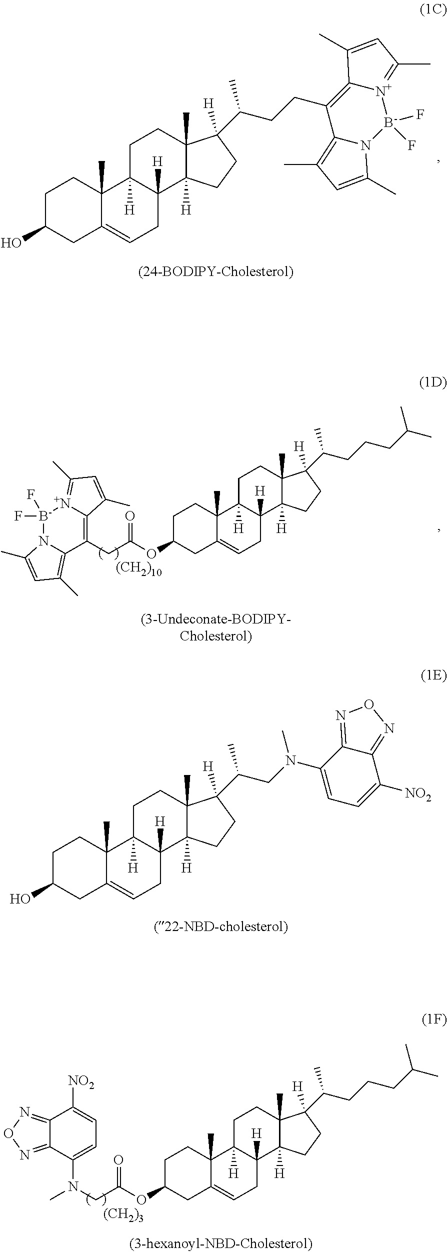

As with dehydroergosterol, the ring-stage parasite culture was incubated with the cholesterol analogues for 24 hours and imaged using deconvolution fluorescence microscopy. 24-BODIPY-cholesterol fluorescence was visible in the RBC membrane of both uRBCs and iRBCs, and in the intracellular parasites in iRBCs. Some fluorescence also appeared to localise within the iRBC cytoplasm, possibly due to incorporation into parasite-induced membranous structures. Fluorescence of 3-undeconate-BODIPY-cholesterol was faint and only visible in the parasite.

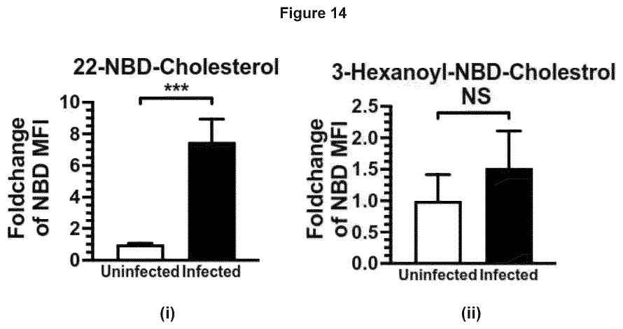

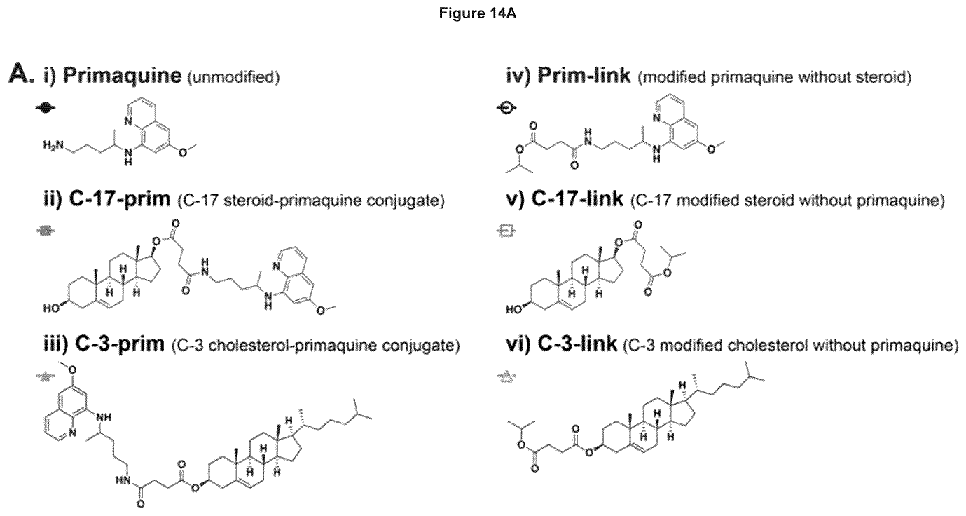

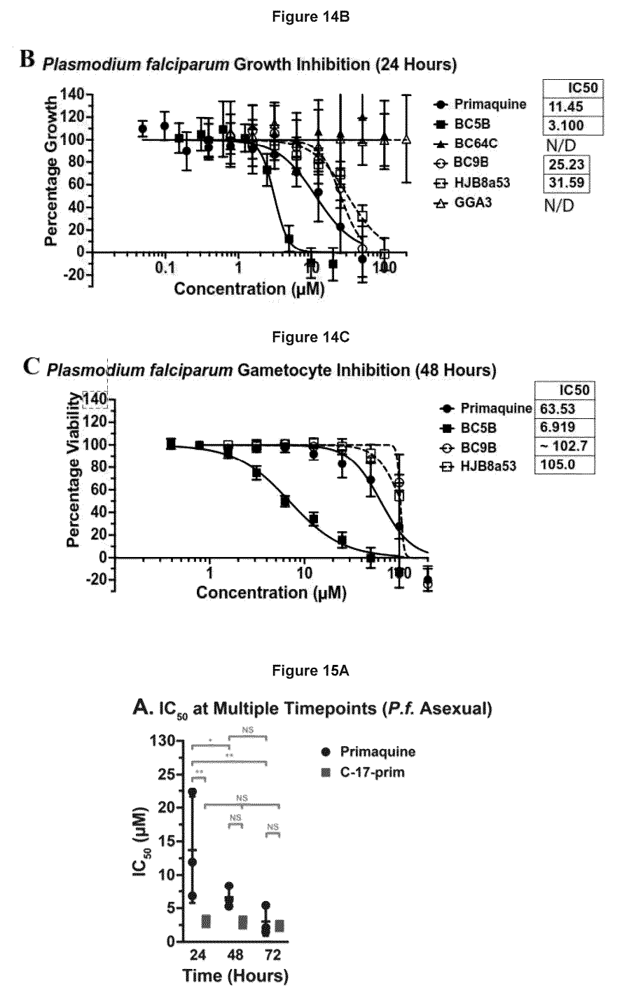

22-NBD-cholesterol fluorescence was bright around the area of the parasite in iRBCs and faintly visible in the RBC membrane of both uRBCs and iRBCs. 3-hexanoyl-NBD-cholesterol fluorescence was faintly visible in the RBC membrane and in the parasite. The differences in fluorescence intensity (relative to unstained controls) between uRBCs and iRBCs were also quantified using flow cytometry. Uptake of 22-NBD-cholesterol was approximately 8-fold higher in iRBCs than uRBCs, while the amount of 3-hexanoyl-NBD-cholesterol taken up was not significantly different between iRBCs than uRBCs (FIG. 14: (i) 22-NBD-cholesterol, and (ii) 3-hexanoyl-NBD-cholesterol).

These results demonstrate that even in the presence of additional bulky molecules fluorescent cholesterol analogues are considerably enriched in the parasite. Differences in fluorescence between uRBCs and iRBCs were most notable when the fluorophore was attached to the cholesterol side chain, and the C-3 hydroxy group was unaltered.

Coupling of the Antimalarial Compound Primaquine to a Steroid Increases its Efficacy and Pharmacological Profile Against Asexual P. falciparum

Conjugating compounds with anti-plasmodial activity to cholesterol or cholesterol-like steroids may result in greater specificity by hijacking the cholesterol uptake pathways to deliver the drug to the parasite. Primaquine was used in this study. Dehydroepiandrosterone (DHEA) is a steroid which is structurally similar to cholesterol, with an identical hydrocarbon ring structure and hydroxy group at C-3. For ease of synthesis, the inventors used DHEA as a chemical surrogate for cholesterol to create a steroid-primaquine conjugate where primaquine is connected from the C-17 position via a succinate linker, termed compound C-17-prim. The inventors attached primaquine via its terminal primary amine in the form of an amide to minimise any disturbance to the mechanism of action, since the active quinoline core is left unchanged. The steroid primaquine conjugate was prepared from DHEA in five steps and 76% overall yield. Synthetic schemes, experimental procedures and characterisation date for synthesised compounds is described below in the section titled “Specific transformations”. For comparison, the inventors conjugated primaquine to the cholesterol C-3 position using the same succinate linking strategy, forming compound C-3-prim. Additionally, the inventors synthesised several control compounds to mimic portions of the full conjugates; primaquine attached to a succinate linker (compound prim-link). DHEA attached to a succinate linker from C-17 (compound C-17-link), and cholesterol attached to a succinate linker on C-3 (C-3-link), with all succinate linkers containing a terminal isopropyl ester. Structures of these compounds are depicted in FIG. 14A.

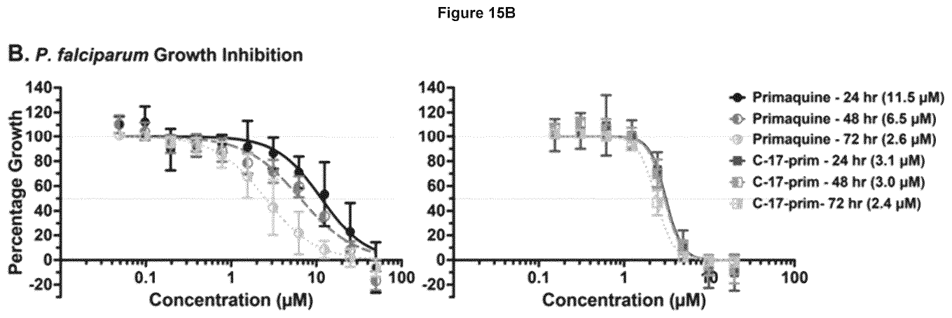

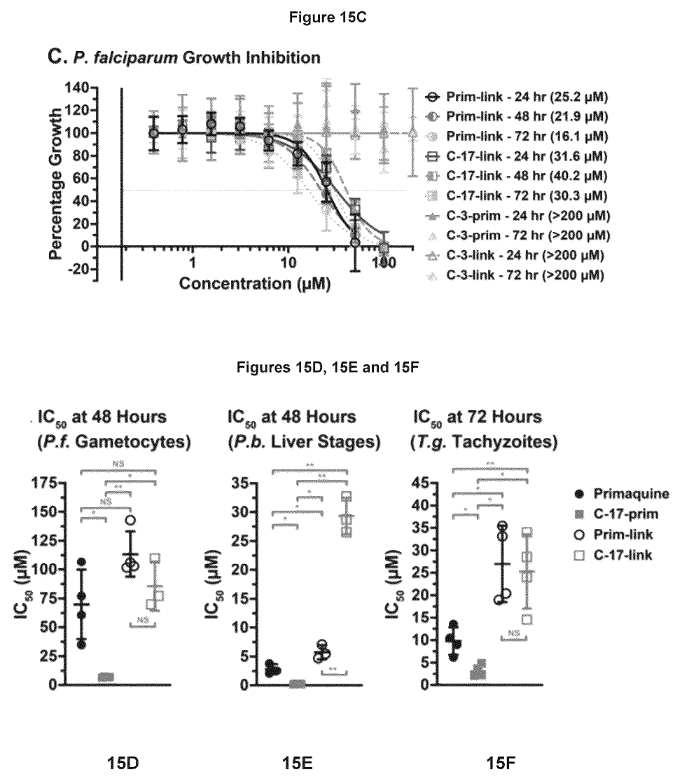

The inventors tested these compounds for inhibitory effects on parasite growth over 24 hours to mimic the conditions of the fluorescent cholesterol uptake assays, starting with ring-stage parasites. The steroid-coupled primaquine exhibited a ˜4-fold lower 50% inhibitory concentration (IC₅₀) than primaquine alone (3.1 μM vs. 11.5 μM; p=0.006) (FIG. 14B and FIG. 15A). In FIG. 14B: BC5B is C-17-prim (DHEA-primaquine conjugate); BC64C is C-3-prim (chol-primaquine conjugate) (>200 μM); BC9B is Prim-link (Primaquine-linker); HJB8a53 is C-17-link (DHEA-linker); GGA3 is C-3-link (Chol-linker) (>200 μM). In addition, the dose response curve is also steeper indicative of different dynamics of the inhibitory action. Hence, the difference between the two compounds is more pronounced (˜8-fold) when comparing IC₉₀ values, that is primaquine, 41 μM and C-17-prim, 5.0 μM (p=0.016). In contrast to C-17-prim, C-3-prim showed no inhibition of parasite growth up to 100 μM. The control compound prim-link showed a higher IC₅₀ than primaquine alone, further supporting the notion that the steroid portion is important for enhanced activity. C-17-link similarly does not show the same level of activity as C-17-prim, indicating that both the drug and the steroid components are important for parasite inhibition, and C-3-link showed no inhibition of parasite growth up to 200 μM.

In order to investigate the dynamics of the drug's effect, the inventors also determined the IC₅₀ of the compounds with longer incubation times of 48 and 72 hours (FIG. 15A). The IC₅₀ of C-17-prim did not change between the different timepoints (p>0.05 for all), indicating that the drug reached its maximum activity after 24 hours. In contrast to C-17-prim, the IC₅₀ of primaquine decreases with longer incubation periods (FIGS. 15A and 15B; p=0.026 24 hours vs 48 hours; p=0.0024 24 hours vs 72 hours), providing further evidence that drug delivery is a limiting factor for primaquine, which can be overcome by coupling to a steroid. While the inhibitory curve for primaquine was steeper at 72 hours compared to the 24 hour exposure time (Hill slope −1.48 and −1.65 for 72 and 24 hours respectively), it remained consistently flatter than the C-17-prim curves at any timepoint (Hill slope −4.20 and −4.56 for 72 and 24 hours respectively). Accordingly, while the IC₅ values after 72 hours exposure time for primaquine approaches the one for C-17-prim, the IC₉₀ values still differ (primaquine, 11.4 μM; C-17-prim, 4.0 μM). None of the control compounds showed significant differences in IC₅₀ between incubation times, nor did the C-3 coupled compounds (FIG. 15C: p>0.05).

Together, these data suggest that the C-17 steroid-primaquine conjugate is a more potent and faster acting drug than primaquine alone against the growth of asexual P. falciparum, indicating that C-17-prim is a lead compound for drug delivery into parasitised RBCs.

Steroid-Primaquine Conjugation Increased Inhibition of P. falciparum Gametocyte Viability

Primaquine also exhibits gametocidal activity. Importantly, gametocytes accumulate large quantities of cholesterol as they develop through stages I-V. The inventors therefore hypothesised that the steroid-primaquine conjugate would show improved activity against the sexual blood stages of P. falciparum, and measured gametocyte viability with a live cell mitochondrial dye (stage III-IV). The steroid-primaquine conjugate exhibited a 9-fold reduction in IC₅₀ compared to primaquine alone after 48 hours of incubation, with an IC₅₀ of 6.9 μM (primaquine 63.5 μM; p=0.025), indicating that the conjugate inhibits gametocyte viability more efficiently (FIG. 14C and FIG. 15D). In FIG. 14C: BC5B is C-17-prim (DHEA-primaquine conjugate); BC9B is Prim-link (Primaquine-linker); HJB8a53 is C-17-link (DHEA-linker). Both of the control compounds, prim-link and C-17-link, showed a significantly higher IC₅₀ (>100 μM) than C-17-prim (p=0.0017 and p=0.023 respectively), indicating that both drug and steroid components are required for enhanced activity. These data indicate that gametocyte inhibition was improved with the steroid conjugation strategy.

The Steroid-Primaquine Conjugate Inhibits the Growth of Liver Stage Plasmodium More Effectively than Primaquine Alone

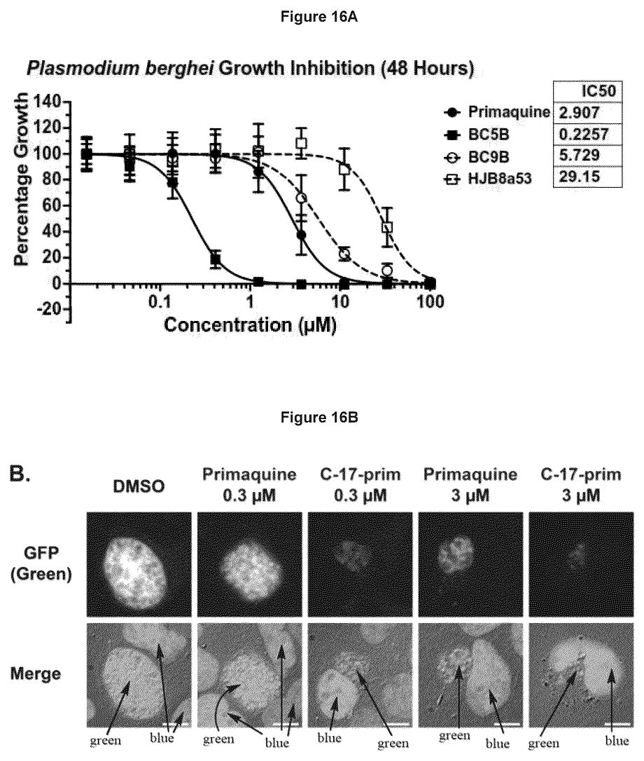

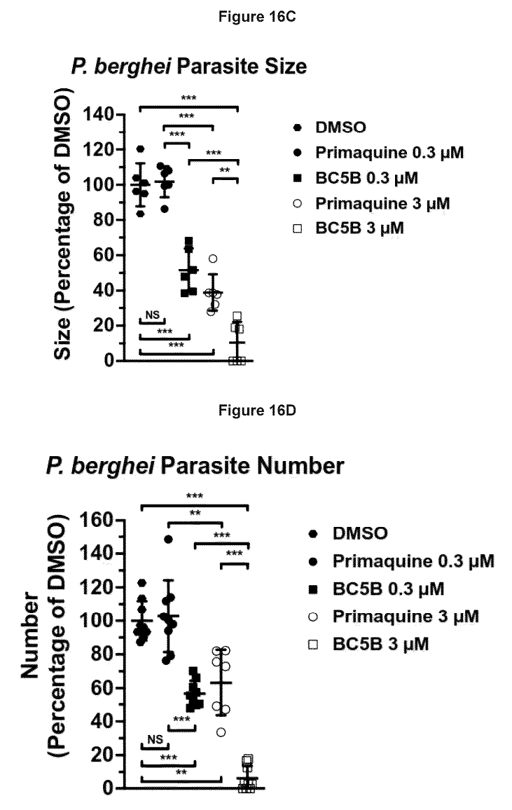

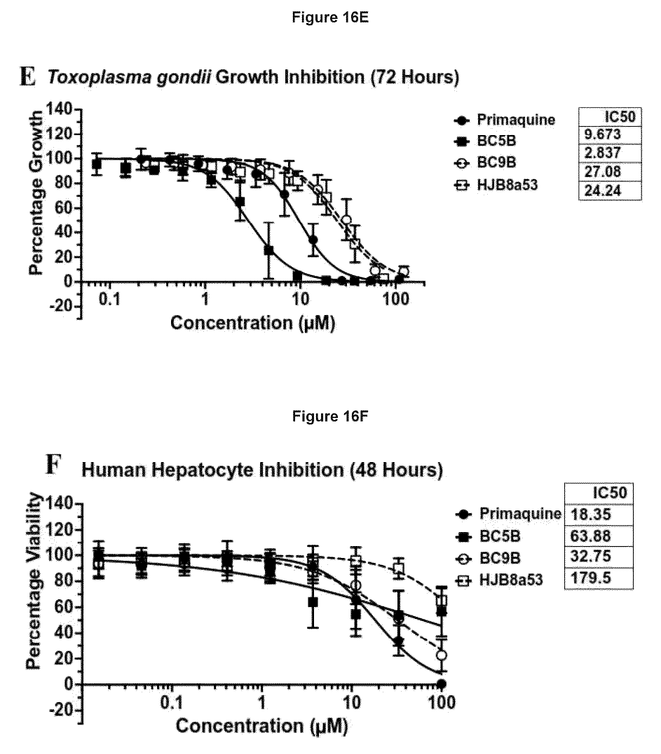

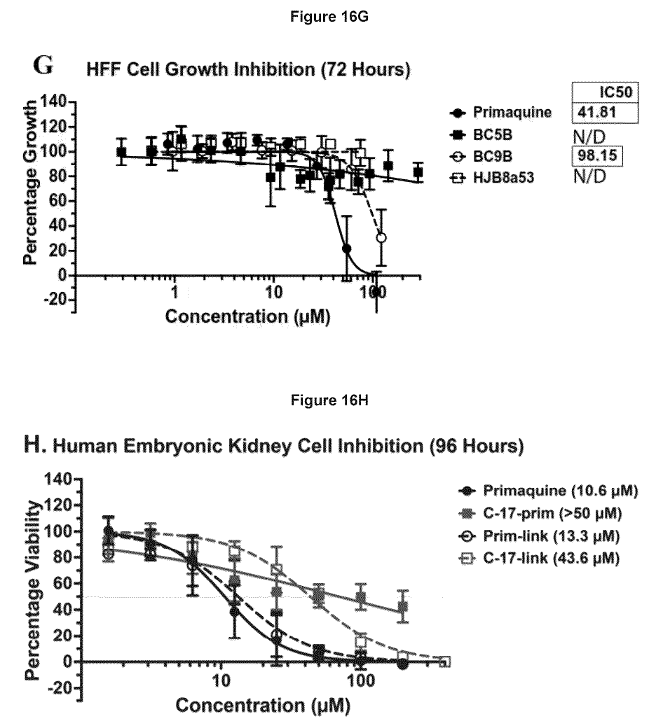

Since primaquine is also used to target liver stage parasites, the inventors investigated if the steroid-primaquine conjugate had any difference in efficacy against this stage. These experiments utilised luciferase-expressing sporozoites of the rodent parasite species Plasmodium berghei to infect human hepatoma cells (Huh7). The steroid-coupled primaquine had an IC₅₀ of 0.2 μM, exhibiting a ˜15-fold reduction in IC₅₀ compared to primaquine alone (2.9 μM; p=0.038) after 48 hours of treatment commencing from sporozoite inoculation (FIG. 16A and FIG. 15E). In FIG. 16A: BCSB is C-17-prim (DHEA-primaquine conjugate); BC9B is Prim-link (Primaquine-linker); HJB8a53 is C-17-link (DHEA-linker). This is particularly interesting because primaquine is oxidised by liver cytochrome P450 2D6 enzymes, producing an activated intermediate which can react further to produce hydrogen peroxide (H₂O₂). Redox cycling of these activated intermediates with liver P450 NADPH-oxidoreductase enzymes then iteratively produces and accumulates reactive oxygen species, leading to parasite death. Therefore, even in the presence of these enzymes that boost primaquine activity, which are produced by the hepatocyte host cells, the C-17-prim conjugate still performs better than primaquine alone. Both of the control compounds, prim-link and C-17-link, showed a significantly higher IC₅₀ than C-17-prim (p=0.016 and p=0.004 respectively).

To better understand the inhibitory activity of the steroid-primaquine conjugate C-17-prim, the inventors conducted fluorescence microscopy experiments to determine the size and number of the parasites inside the liver cells after inoculation with a constant number of sporozoites. There were noticeable differences in both the size and number of the resulting liver stage parasites upon treatment with either primaquine or C-17-prim (FIG. 16B). Quantification of parasite-associated fluorescence signal (FIG. 16C) revealed a smaller parasite size when treated with 0.3 μM C-17-prim (48% reduction, p<0.001), 3 μM C-17-prim (90% reduction, p<0.001) or with 3 μM primaquine (61% reduction, p<0.001) but not 0.3 μM primaquine (p=0.78). This represented a significant difference between C-17-prim and primaquine at the matching concentration (p=0.0013 for 3 μM; p<0.001 for 0.3 μM). Similarly, the parasite number (per coverslip) was significantly lower when treated with C-17-prim compared to the same concentration of primaquine, or the DMSO-only control (p<0.001 for all), again indicating that the conjugate performed better than primaquine alone (FIG. 16D). In FIGS. 16C and 16D: DMSO is dimethylsulfoxide; BC5B is C-17-prim (DHEA-primaquine conjugate).

Together, these data indicate that liver stage inhibition was improved with the conjugation strategy, decreasing the parasite burden.

The Steroid-Primaquine Conjugate Inhibits the Growth of Toxoplasma gondii

Toxoplasma gondii is an Apicomplexan parasite that is related to P. falciparum, and causes toxoplasmosis in humans and animals. T. gondii is less susceptible to primaquine than Plasmodium and this drug is not used for the treatment of toxoplasmosis. Like Plasmodium, T. gondii relies on the uptake of cholesterol to survive. As a proof-of-concept for enhanced uptake by conjugating a steroid to primaquine, the inventors investigated whether the steroid-primaquine conjugate could inhibit the growth of T. gondii tachyzoites, the parasite stage which causes the disease.

The inventors found that the steroid-primaquine conjugate exhibited a significantly lower IC₅ than unconjugated primaquine against T. gondii (2.8 μM vs. 9.7 μM; ˜3.5-fold difference; p=0.015) (FIG. 16E and FIG. 15F). In FIG. 16E: BC5B is C-17-prim (DHEA-primaquine conjugate); BC9B is Prim-link (Primaquine-linker); HJB8a53 is C-17-link (DHEA-linker). Both primaquine and the steroid-primaquine conjugate were more potent than either of the control compounds, demonstrating that both the steroid and drug components are required to effectively inhibit growth.

### Steroid-Primaquine Conjugate Shows Lower Cytotoxicity Against Human Cells

When assessing a potential antiparasitic drug, it is important to consider not only the effect on the parasite, but also the host cells, to determine the compound selectivity toward the parasite and to gauge a safe therapeutic window. Primaquine has a relative small therapeutic window, resulting in an extended therapeutic scheme. The inventors therefore investigated whether the compounds were toxic to three selected cell types: human hepatoma cells (FIG. 16F), human fibroblasts (FIG. 16G), and human embryonic kidney cells (FIG. 15G). In human hepatoma cells (used for P. berghei infection), the inventors detected a gradual effect of C-17-prim over a large range of concentrations; although cell viability was always >50%, an effect on cellular integrity was detected a 3 μM (FIG. 16F). In FIG. 16F: BC5B is C-17-prim (DHEA-primaquine conjugate); BC9B is Prim-link (Primaquine-linker); HJB8a53 is C-17-link (DHEA-linker). Primaquine and prim-link inhibited the viability of the hepatic cells with an IC₅₀ of 18.4 and 32.8 μM respectively, while the steroid-linker compound showed only modest cytotoxicity at high concentrations (>30 μM).

In human foreskin fibroblasts (HFF cells, used for T. gondii infection), the steroid-primaquine conjugate was not able to inhibit more than 30% of the fibroblast growth at the range of concentrations tested, but there was a substantial effect at 10 μM (FIG. 16G). In FIG. 16G: BC5B is C-17-prim (DHEA-primaquine conjugate); BC9B is Prim-link (Primaquine-linker); HJB8a53 is C-17-link (DHEA-linker). Primaquine and prim-link inhibited fibroblast growth with an IC₅₀ of 41.8 μM and 98.2 μM respectively, while no notable inhibition was observed with C-17-link in the range of concentrations tested. Cytotoxicity against human embryonic kidney cells (HEK293) showed similar results, with C-17-prim again showing a gradual cytotoxicity emerging at 10 μM FIG. 16H. Primaquine and prim-link showed IC₅₀ values of 10.6 and 13.3 μM, respectively. C-17-link inhibited HEK293 cells with an IC₅₀ of 43.6 μM.

Together, these data show that steroid-coupling can also enhance the effectiveness of primaquine in another Apicomplexan parasite, T. gondii, whilst still showing lower cytotoxicity to the human host cells.

### Steroid-Artesunate Conjugation Overcomes Drug Resistance in Plasmodium Ring Stages

One of the largest challenges in malaria control today is the emergence and growing prevalence of parasite resistance to frontline treatments. Artemisinin combination therapies (ACTs) are the main anti-malarial therapy in many parts of the world and often the drug of last resort, but emergence and spread of mutations in the parasite protein Kelch-13 results in delayed clearance meaning that there is growing concern that treatment failures will develop. The management of ACT-resistant parasites requires longer treatment times, which impacts on cost and compliance with the treatment regime, and increases the chance of patient morbidity and mortality before the infection can be cured. The inventors therefore investigated whether their conjugation strategy could increase the effectiveness of an artemisinin derivative, artesunate, against parasites resistant to this drug.

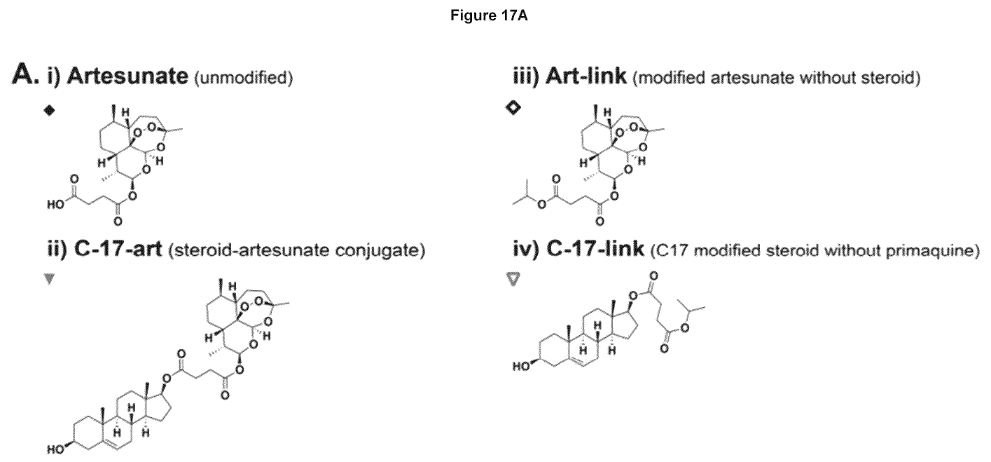

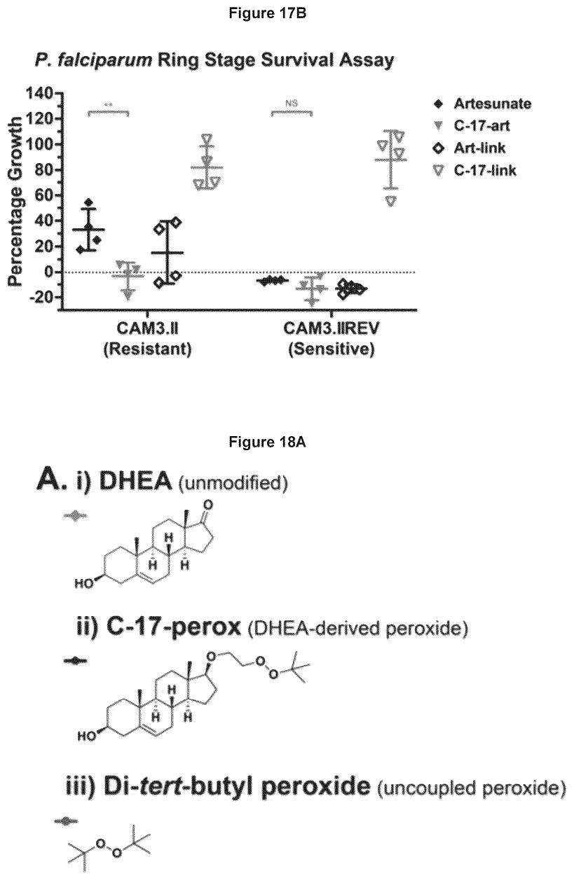

The inventors synthesised a steroid-artesunate conjugate, compound C-17-art, in two steps and 31% overall yield from artesunate (see Chemical Synthesis below) by attaching the succinate group of artesunate to the C-17 position of DHEA (FIG. 17A). Similar to prim-link, the inventors also synthesised a control compound, art-link, where a terminal isopropyl ester group was added to the succinate group of artesunate as a chemical linker. The steroid-linker conjugate, C-17-link, served as the other control.

The inventors tested these compounds in a ring stage survival assay, which allows the detection of resistant parasites against this slow killing drug and is the standard method for investigating resistance to artemisinin and its derivatives (Witkowski B, Menard D, Amaratunga C. Fairhurst R. Ring-stage Survival Assays (RSA) to evaluate the in-vitro and ex-vivo susceptibility of Plasmodium falciparum to artemisinins Procedure. Inst Pasteur du Cambodge—Natl Institutes Heal [Internet]. 2013 Sep. 11 [cited 2022 Jun. 19]; Procedure; available from: https://www.wwarn.org/sites/default/files/INV10-Standard-Operating-Procedure-Ring-Stage-Survival-Assays.pdf). This assay involves a short treatment window against newly-invaded ring stage parasites, followed by removal of drug pressure to mimic the clearance of the drug from plasma. We utilised two parasite strains: an isolate from Cambodia (CAM3.II) with a mutated Kelch-13 conferring resistance, and a genetically modified revertant of this strain (CAM3.IIREV) conferring drug susceptibility (Straimer J, Gnädig N F, Witkowski B, Amaratunga C, Duru V, Ramadani A P, et al. K13-propeller mutations confer artemisinin resistance in Plasmodium falciparum clinical isolates. Science (80-) [Internet]. 2015 Jan. 23 [cited 2022 Jun. 19]; 347(6220):428-31; available from: https://www.science.org/doi/10.1126/science.1260867).

Giemsa-stained smears of the resistant CAM3.II strain treated with artesunate or art-link showed a combination of morphologically normal parasites and morphologically aberrant pyknotic or vacuolated forms, indicating loss of viability. In contrast, upon treatment with C-17-art, only aberrant, non-viable parasites were visible. Giemsa smears of the susceptible CAM3.IIREV strain confirmed that only morphologically aberrant parasites were present when treated with artesunate, C-17-art, or art-link (FIG. 17B). Thus, C-17-art kills all cells of the artesunate-resistant P. falciparum strain CAM3.II, whereas >30% of these cells survive in the presence of free artesunate when tested in a ring stage survival assay. This indicates a higher efficacy of C-17-art against artesunate-resistant cells compared to artesunate alone.

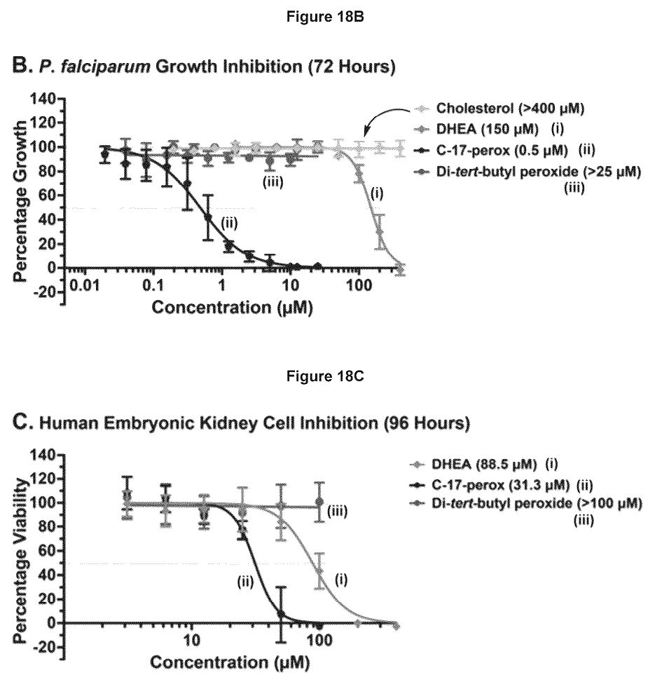

The inventors used DNA replication to measure parasite survival, revealing that a large proportion (˜30%) of the resistant parasites survived artesunate treatment. In marked contrast, complete growth inhibition was observed with the steroid-artesunate conjugate (p=0.002 vs artesunate; p<0.001 for each vs DMSO solvent control; FIG. 18C). In the susceptible control strain, all artesunate-containing compounds displayed complete growth inhibition (p<0.001 for all compared to DMSO solvent control), whereas the steroid-linker compound C-17-link did not significantly impact on the growth of either strain (p=0.08 and p=0.22 for resistant and susceptible strains respectively). It was also noted that addition of an ester group to artesunate (compound art-link) showed a trend to improved efficacy over artesunate.

Together, these data show that the conjugate was more effective against resistant parasites than the free drug, providing evidence that the steroid conjugation strategy may help to overcome drug resistance.

Novel Steroid-Peroxide Compound which Mimics Cholesterol Inhibits P. falciparum Growth

Synthetic reactive groups such as trioxolane have been shown to effectively kill intra-erythrocytic P. falciparum parasites. Here, the inventors took inspiration from this and established antimalarial drugs with peroxides (such as artesunate) and coupled DHEA to a side chain containing a dialkyl peroxide (compound C-17-perox) in five steps and 26% overall yield in a way that more closely resembles the native structure of cholesterol (FIG. 18A). For comparison, we used di-tert-butyl peroxide to establish any general peroxide-dependent effects.

The inventors tested these compounds and steroid controls for inhibitory effects on P. falciparum growth (FIG. 18B). It was found that the steroid-bound peroxide inhibited parasite growth with an IC₅₀ of 480 nM. Neither cholesterol nor di-ter-butyl peroxide inhibited parasite growth within the range of concentrations tested (IC₅₀>400 μM and 25 μM respectively), and DHEA exhibited an IC₅₀ of 150 μM. This suggests that a peroxide group capable of generating reactive oxygen species can be delivered to the intracellular parasite by conjugating it to steroids. The inventors also tested cytotoxicity against HEK293 cells, finding a ˜60-fold difference in the C-17-perox IC₅₀ compared to P. falciparum (31.3 μM vs 0.5 μM), indicating good selectivity (FIG. 18C).

### Discussion

In this study, the inventors investigated the uptake of cholesterol into P. falciparum parasites, and devised a strategy which uses this pathway to efficiently deliver antimalarial drugs. This work represents a previously unrecognized way of exploiting Apicomplexan cholesterol autotrophy as an Achilles' heel for targeting intracellular parasites. Instead of disrupting the movement or metabolism of molecules, the inventors have instead sought to hijack an essential uptake pathway as a system for effectively delivering molecules with anti-parasitic properties that work against a range of targets, and holds promise for a range of pathogens and diseases that remain difficult to treat, including Plasmodium and T. gondii.

The inventors found that coupling the antimalarial drug primaquine to a cholesterol-mimicking steroid resulted in a compound that improves efficacy against Plasmodium parasites at three different lifecycle stages—asexual intraerythrocytic parasites, sexual gametocyte stages, and liver stages—at lower concentrations, while also showing lower cytotoxic effects to human cell lines. Strategies that are effective against multiple stages of parasites are highly desirable in the fight against these diseases.

The use of primaquine as an antimalarial treatment or preventative is limited by its slow action and high toxicity. Steroid-coupled primaquine inhibits parasites more effectively than the free drug and therefore could allow for lower doses and address safety concerns associated with primaquine toxicity.

The inventors also noted that the shape of the drug inhibition curve was much steeper with the C-17-prim conjugate compared to primaquine alone. Having a larger concentration range where some parasites survive drug exposure (exhibited by a flatter dose-response curve) indicates a potential higher risk of the emergence of drug-resistant parasites. Improved delivery of drug to the parasite results in the conjugate acting more efficiently than primaquine alone. Increased killing speed could provide an advantage for fast parasite clearance and again decreases the chance of resistant parasites emerging.

The activity of primaquine is enhanced in the presence of P450 NADPH-oxidoreductase produced in the liver and bone marrow, which forms intermediates which react with molecular oxygen to produce H₂O₂, causing oxidative damage to the parasites. While P. falciparum experiments were performed in the absence of P450 NADPH-oxidoreductase, P. berghei liver stage assays were done in hepatoma cells that express this enzyme. These data illustrate that the steroid-primaquine conjugate C-17-prim can be more effective than primaquine even in the presence of this enzyme, which supports the notion that the steroid conjugation strategy could be advantageous for improving in vivo primaquine action.

The inventors found that coupling the antimalarial drug artesunate to a steroid improved its efficacy against parasites resistant to this drug, providing evidence that a delivery system may help to overcome drug resistance. The improved action of C-17-art against the resistant parasites is potentially due to increased compound uptake within the short treatment window, and/or increased compound retention within the cell after removal from the extracellular environment. Since artesunate has a very short half-life in blood (0.5-1.5 hours), either of these changes would pose an advantage for greater treatment efficacy against susceptible and resistant parasites. The C-17 steroid-artesunate conjugate holds promise to improve efficacy both against susceptible and resistant parasites.

The inventors have presented evidence that this delivery system is broadly applicable across not only multiple lifecycle stages but across multiple species of parasites, exemplified by T. gondii, where the steroid-primaquine conjugate showed a 3-fold lower IC₅₀ than primaquine. There are many drugs or drug-like compounds that are effective against Plasmodium, but not Toxoplasma or other closely related parasites, despite a high number of closely conserved genes. In some cases, this is due to lower permeability and drug uptake, preventing these compounds from accessing their target site. Therefore, the drug delivery system may be especially useful, since antimalarial drugs could be repurposed for toxoplasmosis and related diseases. In addition, many other (non-Apicomplexan) parasites also rely on the uptake of host cholesterol, and so the delivery system might also relevant to these parasites.

Also described is a strategy of directly modifying steroids into drug-like compounds by attaching peroxides directly onto the steroid core. These compounds likely take advantage of the parasite's increased susceptibility to oxidative damage by delivering a molecule that may produce reactive oxygen species. These compounds have a lower IC₅₀ than primaquine against asexual P. falciparum, highlighting their potential as a therapeutic compound.

This study also provided insights into the structural requirement for parasite cholesterol uptake: fluorescent cholesterol conjugate uptake was higher when the fluorophore was conjugated from the C-17-linked side chain of cholesterol, leaving the C-3 hydroxy group unmodified. This difference in uptake based on conjugation sites was consistent in the activity of the steroid-primaquine conjugates. The C-3 hydroxy group has previously been implicated as important in the recognition of cholesterol by human transport enzymes, as well as a wide range of cholesterol-binding proteins from various species. Judging from the present inventors' data, having an unmodified C-3 hydroxy group seems to also be important for the uptake or trafficking to the parasite. This requirement might also explain why attempts to conjugate drugs to the C-3 group of cholesterol were not successful. The importance of the free C3 hydroxy group is further corroborated by the finding that the effects of cholesterol depletion on Plasmodium could not be recovered with reconstitution using epicholesterol, which differs to cholesterol in stereochemistry at the C-3 site. Together, this suggests that the stereochemical configuration of the C-3 hydroxy group is an important feature of cholesterol for parasite development, thus conjugating compounds on or after the C-17 of the cholesterol sidechain is a more suitable method for drug delivery.

In conclusion, conjugating anti-parasitic moieties to a steroid backbone in order to exploit cholesterol uptake by Plasmodium for drug delivery improved the efficacy of current antimalarial drugs, primaquine and artesunate. The strategy could be broadly applicable to a wide variety of parasites which rely on cholesterol scavenging from the host.

### Materials and Methods

### Ethics Statement:

All relevant aspects of this study were approved by the Australian National University's Human Ethics Committee, procedure HEC2017/351, or the Berlin Ethics Committee (Landesamt für Gesundheit und Soziales Berlin), permit G0294/15, in strict adherence to German and European Union animal protection laws. Human red blood cell and serum were kindly provided by the Australian Red Cross Blood Service (“Lifeblood”). Donor consent was obtained as part of the donation process.

### Chemical Synthesis:

The steroid drug conjugates and related control compounds were prepared by chemical synthesis, with compound identity and purity (>95%) confirmed for all materials prior to biological evaluation. Reaction schemes, detailed experimental procedures, purity and characterisation data for synthesised materials are provided below.

### Cell Culture:

### Human Hepatoma (Huh7) Culture

The Huh7 hepatoma cell line was maintained under routine culture conditions in DMEM containing 3.7 g/L NaHCO3 supplemented with 10% FBS, 100 units/mL penicillin, and 100 μg/mL streptomycin at 37° C. in a humidified 5% CO₂ incubator.

### Human Embryonic Kidney Cell (HEK293) Culture

The HEK293 cell line was maintained under routine culture conditions in DMEM containing 3.7 g/L NaHCO3 supplemented with 10% FBS, 200 μM glutamine, 100 units/mL penicillin, and 100 μg/mL streptomycin at 37° C. in a humidified 5% CO₂ incubator.

### Human Foreskin Fibroblast (HFF) Culture:

The HFF cell line was maintained under routine culture conditions in DMEM containing 3.7 g/L NaHCO3 supplemented with 10% v/v newborn calf serum (NCS), 50 units/mL penicillin, 50 μg/mL streptomycin, 10 μg/mL gentamicin, 0.25 μg/mL amphotericin B. and 0.2 mM L-glutamine at 37° C. in a humidified 5% CO₂ incubator.

Toxoplasma gondii Culture:

RH Toxoplasma gondii parasites expressing the fluorescent protein tdTomato (Rajendran et al., 2017) were cultured under routine culture conditions in HFF cells and DMEM containing 2 g/L NaHCO3 supplemented with 1% (v/v) FBS, 50 units/mL penicillin, 50 μg/mL streptomycin, 10 μg/mL gentamicin, 0.25 μg/mL amphotericin B, and 0.2 mM L-glutamine at 37° C. in a humidified 5% CO₂ incubator.

### Plasmodium Culture:

P. falciparum parasites were maintained under routine culture conditions in red blood cells and RPMI 1640-HEPES with GlutaMAX™, supplemented with 10 mM D-glucose, 480 μM hypoxanthine, 20 μg/mL gentamicin, 0.375% (w/v) AibuMAX™ II, and 2.5% v/v heat-inactivated human serum.

All experiments used P. falciparum 3D7 wildtype parasites unless otherwise specified. Gametocyte formation was induced as described (Ridgway M C, Shea K S, Cihalova D, Maier A G. Novel Method for the Separation of Male and Female Gametocytes of the Malaria Parasite Plasmodium falciparum That Enables Biological and Drug Discovery. mSphere [Internet]. 2020 Aug. 26 [cited 2022 Jun. 6]; 5 (4):e00671-20). Plasmodium berghei ANKA Bergreen (Kooij T W A, Rauch M M, Matuschewski K. Expansion of experimental genetics approaches for Plasmodium berghei with versatile transfection vectors. Mol Biochem Parasitol [Internet]. 2012 September [cited 2022 Jun. 6]; 185(1):19-26) or GFP-Luc (RMgm-29) (Janse C J, Franke-Fayard B, Mair G R, Ramesar J, Thiel C, Engelmann S, et al. High efficiency transfection of Plasmodium berghei facilitates novel selection procedures. Mol Biochem Parasitol [Internet]. 2006 January [cited 2022 Jun. 6]; 145(1):60-70), respectively expressing green fluorescent protein (GFP) or a GFP-Luciferase fusion protein, were maintained under routine conditions in SWR/J Mus musculus mice and Anopheles stephensi mosquitos.

### Uptake of Fluorescent Cholesterol Analogues:

In brief, ring-stage parasites at 4% parasitemia and 2% haematocrit were incubated with 2 μM fluorescent cholesterol analogue (dehydroergosterol, 24-BODIPY-Cholestrol, 3-Undeconoate-BODIPY-Cholesterol, 22-NBD-Cholesterol, or Hexanoyl-NBD-cholesterol) or solvent controls in cholesterol-depleted media for 24 hours at 37° C. under standard culturing conditions. Cells were washed twice in PBS with 10 mM D-Glucose (PBS-G), and then resuspended in 5 μg/mL Hoechst 33342 (Thermo Fisher 14533) in PBS-G and incubated for 20 minutes at 37° C. Cells stained with dehydroergosterol were instead incubated with 500 nM Mitotracker™ Deep Red FM (Thermo Fisher M22426) for 15 minutes and then washed twice in PBS-G. Samples were imaged with deconvolution fluorescence microscopy (1000× magnification) or read on a flow cytometer, using Hoechst fluorescence to differentiate uRBCs (negative) and iRBCs (positive). For each fluorescent cholesterol analogue, images were collected under the same exposure conditions (without binning) and converted to TIFF files under the same brightness and contrast settings. Individual cells were cropped from larger images with Fiji ImageJ. No other manipulations were performed. Flow cytometry data was normalised by subtracting background (unstained) fluorescence and by setting the fluorescence of uRBCs to 1.

### Growth Inhibition and Viability Assays:

Growth inhibition or cell viability were assessed by incubation with a serial dilution of test compounds, solvent controls (DMSO, ethanol, ethyl acetate), or culture media alone in 96-well plates, with detection of fluorescence, luminescence, or absorbance in microplate readers after 24-96 hours unless otherwise specified.

P. falciparum asexual parasite assays begun with ring-stage parasites at 1% parasitaemia and 1% haematocrit. 200 nM chloroquine (Sigma C6628) was used as a no-growth control. DNA replication (indicating growth) was measured at the specified time using SYBR Safe DNA Gel Stain (Invitrogen 33102) (Smilkstein M, Sriwilaijaroen N, Kelly J X, Wilairat P, Riscoe M. Simple and inexpensive fluorescence-based technique for high-throughput antimalarial drug screening. Antimicrob Agents Chemother [Internet]. 2004 May 1 [cited 2017 Oct. 17]; 48(5):1803-6; Spry C, Macuamule C, Lin Z, Virga K G, Lee R E, Strauss E. et al. Pantothenamides Are Potent, On-Target Inhibitors of Plasmodium falciparum Growth When Serum Pantetheinase Is Inactivated. Ralph S A, editor. PLoS One [Internet]. 2013 Feb. 6 [cited 2017 Oct. 17]; 8 (2):e54974).

Gametocyte assays begun eight days post-invasion (Stage III-IV). 100 mM artemisinin (Sigma 361593) was used as no-viability control. Mitochondrial membrane potential (indicating viability) was measured using flow cytometry after staining with 500 nM MitoTracker™ Deep Red FM and 5 μg/ml Hoechst 33342 to detect DNA (Ridgway M C, Shea K S, Cihalova D, Maier A G. Novel Method for the Separation of Male and Female Gametocytes of the Malaria Parasite Plasmodium falciparum That Enables Biological and Drug Discovery. mSphere [Internet]. 2020 Aug. 26 [cited 2022 Jun. 6]; 5 (4):e00671-20).

Huh7 cells were seeded at a density of 10 000 cells per well, or 20 000 per collagen-coated coverslip. After 24 hours, test compounds were added the wells were inoculated with P. berghei liver stage sporozoites dissected from infected A. stephensi salivary glands with 5000-10 000 sporozoites per well or 10 000-20 000 sporozoites per coverslip. Uninfected wells served as a control for background luminescence. Luminescence signal was measured 48 hours post-invasion by addition of 1:1 ONE-Glo™ substrate (Promega). Coverslips were fixed, stained, and imaged, with all parasites on each coverslip imaged and delineated as described (Petersen W, Matuschewski K, Ingmundson A. Trafficking of the signature protein of intra-erythrocytic Plasmodium berghei-induced structures, IBIS1, to P. falciparum Maurer's clefts. Mol Biochem Parasitol [Internet]. 2015 May 30 [cited 2022 Jun. 6]; 200 (1-2):25-9) using Fiji ImageJ (Schindelin J. Arganda-Carreras I, Frise E, Kaynig V. Longair M. Pietzsch T. et al. Fiji—an Open Source platform for biological image analysis. Nat Methods [Internet]. 2012 July [cited 2022 Jun. 6]; 9(7):676-82). Data are presented as a percentage of the size or parasite number of the solvent control to normalize for differences between experiments. For cell images presented in the figure, images were collected under optimal exposure settings to ensure features were visible. Individual cells were cropped from larger images with Fiji ImageJ, and the brightness was adjusted while ensuring that no fluorescence was removed. No other manipulations were performed.

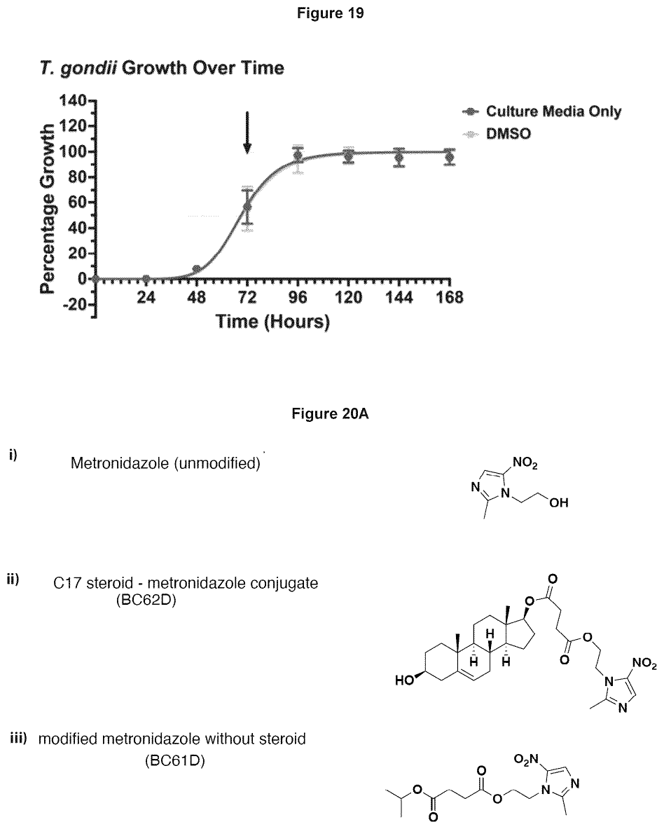

T. gondii tachyzoite parasite assays begun with 2 000 RH tachyzoites expressing the fluorescent protein tdTomato per well containing confluent HFF cells (Rajendran E, Hapuarachchi S V., Miller C M, Fairweather S J, Cai Y, Smith N C, et al. Cationic amino acid transporters play key roles in the survival and transmission of apicomplexan parasites. Nat Commun 2017 81 [Internet]. 2017 Feb. 16 [cited 2022 Jun. 6]; 8:1-4455). Fluorescence was measured daily with a FluoStar Optima fluorescence plate reader for one week, and the background fluorescence from the time 0 reading was subtracted from all other measurements. The 72-hour timepoint was chosen for further analysis because parasite growth was in the mid-logarithmic stage (FIG. 19).

Huh7 hepatoma viability assays were conducted in the same manner as the P. berghei assays, with metabolic capacity (indicating viability) measured by incubation with CellTiter-Blue® (Promega) for 3 hours.

HFF cell growth inhibition assays began with 5000 cells per well. 10 μg/mL cycloheximide was used as a no-growth control. DNA replication (indicating growth) was measured at the specified time using SYBR Safe DNA Gel Stain (Invitrogen 33102).

HEK293 cell viability assays began with 5000 cells per well. 10 μg/mL cycloheximide was used as a no-growth control. Metabolic capacity (indicating viability) was measured by incubation with 0.45 mg/mL methylthiazolyldiphenyl-tetrazolium bromide (MTT; Sigma M2128) for two hours followed by crystal solubilisation in SDS.

Plasmodium falciparum Ring Stage Survival Assay

Following tight synchronisation, CAM3.II and CAM3.IIREV ring stage parasites (0-3 hours post invasion) at 1% parasitaemia and 2% haematocrit were incubated with 700 nM of compounds or solvent-only controls for exactly six hours, before removing the drug, thoroughly washing in RPMI, and returning to culture conditions for a further 66 hours. Some cells were treated with 700 nM artesunate for 72 hours as a no-growth control. DNA replication (indicating growth) was measured at the specified time using SYBR Safe DNA Gel Stain. Microscope slide smears of each condition were stained with 10% v/v Giemsa and examined under a light microscope (1 000× magnification); with ‘viable’ (morphologically normal) and ‘non-viable’ (morphologically aberrant; vacuolated or pyknotic) parasites distinguished according to standard protocol (Witkowski B, Menard D, Amaratunga C, Fairhurst R. Ring-stage Survival Assays (RSA) to evaluate the in-vitro and ex-vivo susceptibility of Plasmodium falciparum to artemisinins Procedure. Inst Pasteur du Cambodge—Natl Institutes Heal [Internet]. 2013 Sep. 11 [cited 2022 Jun. 19]; Procedure. Available from: https://www.wwam.org/sites/default/files/INV10-Standard-Operating-Procedure-Ring-Stage-Survival-Assays.pdf). Images were captured using a Leica ICC50 camera, with a resolution of 76 nm per pixel.

### Growth Inhibition Data:

Growth inhibition data were normalised by subtracting background fluorescence, luminescence, absorbance, or percentage of cells from a no-growth or no-viability control (high drug concentration, no-parasite control, or time=0 measurement as specified), and expressed as a percentage of the growth or cell percentage in the negative control (culture media alone or solvent control) at the indicated timepoint (or to the highest measurement reached during the assay for FIG. 15B). Data were fitted with a four parameter [inhibitor] vs response curve in Prism 9, and the concentration inhibiting 50% of growth (IC₅₀) was calculated by the model.

### Statistics:

Except where otherwise specified, data were analysed in Prism 9 using one-way or two-way ANOVA with corrections for the false discovery rate using the two-stage setup method of Benjamini et al. (Benjamini Y, Kreiger A M, Yekutieli D. Adaptive linear step-up procedures that control the false discovery rate. Biometrika [Internet]. 2006; 93(3):491-507. Available from: http://www.math.tau.ac.il/˜yekutieVpapers/KBY—adaptive FDR.pdf).

### Chemical Synthesis

### Experimental

Unless otherwise specified ¹H NMR and ¹³C NMR experiments were performed using deuterated chloroform (CDCl₃) or deuterated methanol (MeOD-d₄) using Bruker Avance 400 MHz, 600 MHz or 700 MHz spectrometers at 298 K. Deuterated solvents supplied by Cambridge Isotope Laboratories, Inc. Residual solvent peaks or ¹³C signals corresponding to CHCl₃ and MeDH₂OD-d₃ were used as internal reference corresponding to values given by Fulmer et al., (2010) (cited as reference 1A in Section 1.3 above). Analysis of these spectra was completed using MestReNOVA (version 14.2.1). Chemical shifts are reported in parts per million (ppm). Multiplicity is assigned as s=singlet, d=doublet, t=triplet, q=quartet, sept=septet and m=multiplet or combination of these. Where compounds were found as a pair of inseparable diastereomers; peaks corresponding to the same ¹H and ¹³C environments are given as peak 1/peak 2. Coupling constants (J) reported in Hz. Unless otherwise stated, low-resolution mass spectrometry (LRMS) was performed using a Waters LCT Premier XE mass spectrometer and high-resolution mass spectrometry (HRMS) was performed using a Waters SYNAPT G2-Si mass spectrometer. Samples were prepared at a concentration of ˜1 mg of analyte in 1 mL of methanol for LRMS and was subsequently diluted in methanol for HRMS. Infrared spectra were recorded using PerkinElmer 1800 Series FTIR spectrometer. Specific rotation recorded using the Rudolf research systems Autopol I polarimeter, where 10 mg of analyte was dissolved in 1.0 mL of chloroform. Thin layer chromatography (TLC) analysis performed using Merck TLC silica gel 60 F254 plates using mobile phases as stated. Purification by silica flash chromatography was conducted using chem-supply silica gel 60 0.04-0.06 mm (230-400 mesh ASTM) using eluent as stated. Purification by high performance liquid chromatography (HPLC) was conducted using Waters 2695 separations module, Agilent Pursuit XRs 5 C18 250×10 mm column, Waters 2998 Photodiode array detector (266 nm) and Waters Fraction Collector Ill, controlled by Waters Empower 2 software. Purity of compounds used for biological testing (>95%) was determined using the same Waters separations module and photodiode array detector with an Agilent Eclipse XDB-C18 5 μm column. General eluent used: 95% HPLC methanol (Honeywell—Burdick & Jackson):5% (0.1% HPLC trifluoroacetic acid) (Sigma-Aldrich) solution in filtered water or 90% Acetonitrile:10% water.

### Specific Transformations

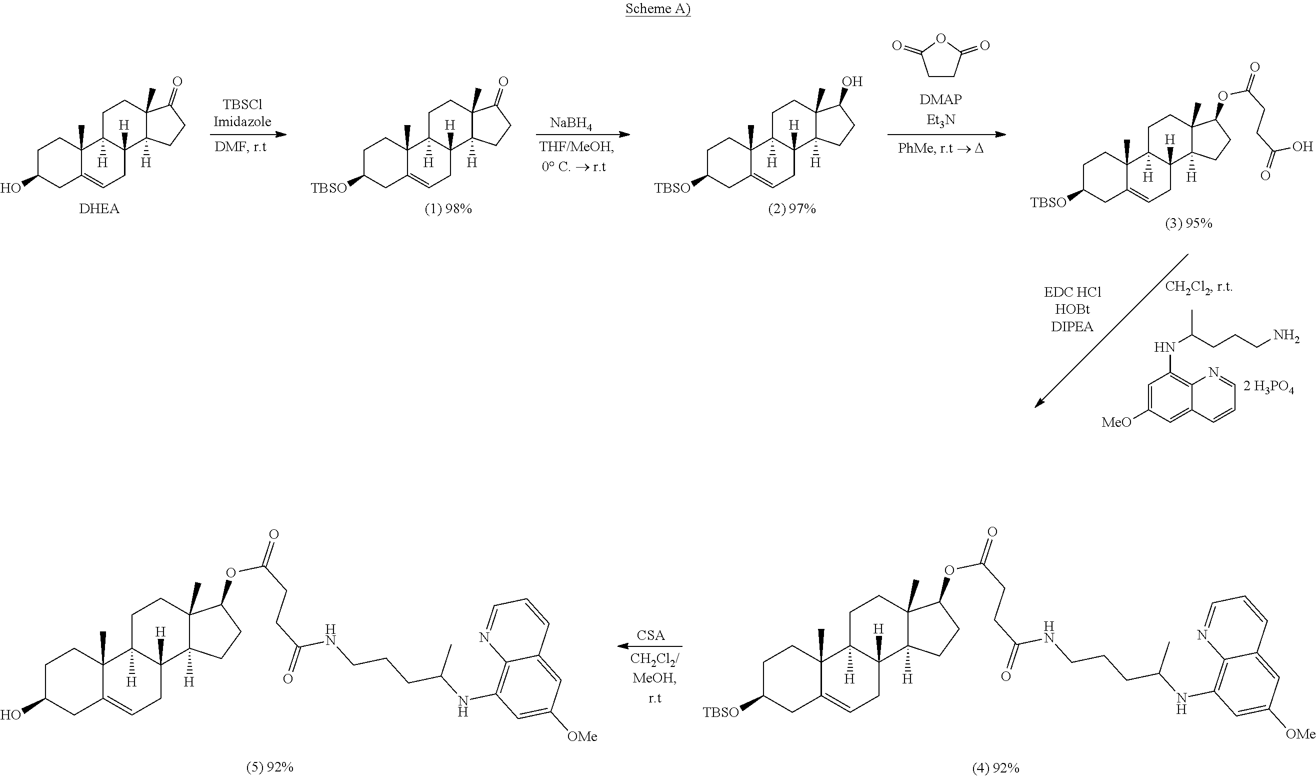

### 3β-(tert-Butyldimethylsilyloxy)-androst-5-en-17-one (1)

Procedure adapted from Yamauchi, T.; Kato, M.; Mikami, T.; Fujimura, Y. Synthesis of 1-Deoxymaxacalcitol. Heterocycles 2005, 65 (9), 2111. tert-Butyldimethylsilyl chloride (2.74 g, 18.2 mmol) was added to a stirring solution of 3β-hydroxyandrost-5-en-17-one (3.50 g, 12.1 mmol) and imidazole (2.07 g, 30.3 mmol) in anhydrous DMF (40 mL). The reaction mixture was stirred at room temperature for 2 hours. The reaction was diluted with EtOAc (100 mL) and 5% citric acid solution (100 mL). The aqueous layer was further extracted with EtOAc (2×100 mL). The combined organic extract was washed with saturated NaCl solution, dried over anhydrous MgSO₄ and filtered. The solvent was removed under reduced pressure and the residue obtained was purified by silica column chromatography (20% EtOAc:n-hexanes) to yield 3β-(tert-butyldimethylsilyloxy)-androst-5-en-17-one (4.79 g. 98%) as a white solid.

m.p 147-149° C. (lit. 149° C.: Yamauchi, T.; Kato, M.; Mikami, T.; Fujimura, Y. Synthesis of 1-Deoxymaxacalcitol. Heterocycles 2005, 65 (9), 2111); ¹H NMR (400 MHz, CDCl₃) δ 5.34 (d, J=5.3 Hz, 1H), 3.48 (m, 1H), 2.40-0.95 (m, 19H), 1.02 (s, 3H), 0.89 (s, 9H), 0.88 (s, 3H), 0.06 (s, 6H); ³C NMR (101 MHz, CDCl₃) δ 221.3, 141.9, 120.5, 72.6, 52.0, 50.5, 47.7, 42.9, 37.5, 36.9, 36.0, 32.2, 31.7, 31.6, 31.0, 26.1, 22.0, 20.5, 19.6, 18.4, 13.7, −4.4 (2C); LRMS (GCMS) found m/z 345 [M−C₄H₉]⁺; HRMS (ESI+): found m/z 425.2834 [M+Na]⁺, theoretical (C₂₅H₄₂O₂Na) m/z 425.2834 [M+Na]⁺; IR 2946, 2857, 1746 cm⁻¹; specific rotation [α]_{D}²⁵−38.63 (c 1.0, CHCl₃).

### 3β-(tert-Butyldimethylsilyloxy)-androst-5-en-17β-ol (2)

Procedure adapted from Moreira, V. M. A.; Vasaitis, T. S.; Guo, Z.; Njar, V. C. O.; Salvador, J. A. R. Synthesis of Novel C17 Steroidal Carbamates: Studies on CYP17 Action. Androgen Receptor Binding and Function, and Prostate Cancer Cell Growth. Steroids 2008, 73 (12). 1217-1227. Sodium borohydride (0.536 g, 14.2 mmol) was added to a stirring solution of 3β-(tert-butyldimethylsilyloxy)-androst-5-en-17-one (2.85 g, 7.08 mmol) in ice bath cooled THF (20 mL) and MeOH (20 mL). The reaction mixture was brought to room temperature and stirred for 2.5 hours. The reaction was diluted with CH₂Cl₂ (40 mL) and water (20 mL), then treated with 2M HCl solution (5 mL). The aqueous layer was further extracted with CH₂Cl₂ (2×50 mL). The combined organic extract was washed with saturated NaCl solution, dried over anhydrous MgSO₄ and filtered. The solvent was removed under reduced pressure and the residue obtained was purified by silica column chromatography (20% EtOAc:n-hexanes) to yield 3β3-(tert-butyldimethylsilyloxy)-androst-5-en-17β-ol (2.78 g, 97%) as a white solid.

m.p 170-172° C. (lit. 171-172° C.: Hosoda, H.; Fukushima, D. K.; Fishman. J. Convenient, High Yield Conversion of Androst-5-Ene-3.Beta.,17.Beta.-Diol to Dehydroisoandrosterone. J. Org. Chem. 1973, 38 (24), 4209-4211); ¹H NMR (400 MHz, CDCl₃) δ 5.31 (d, J=4.6 Hz, 1H), 3.64 (t, J=8.5 Hz, 1H), 3.52-3.42 (m, 1H), 2.30-0.93 (m, 19H), 1.01 (s, 3H), 0.89 (s, 9H), 0.75 (s, 3H), 0.05 (s, 6H), OH not observed; ¹³C NMR (101 MHz, CDCl₃) δ 141.8, 121.0, 82.1, 72.7, 51.5, 50.5, 43.0, 42.9, 37.6, 36.8, 36.8, 32.2, 32.1, 31.7, 30.7, 26.1, 23.6, 20.8, 19.6, 18.4, 11.1, −4.4 (2C); LRMS (GCMS) found m/z 347 [M−C₄H₉]⁺; HRMS (ESI+) found m/z 427.2992 [M+Na]⁺, theoretical (C₂₃H₄₄O₃Na) m/Z 427.3002 [M+Na]⁺; IR 3305, 2929, 2853 cm⁻¹; specific rotation [α]_{D}²⁵−38.63 (c 1.0, CHCl₃)

### 3β-(ter-Butydimethylsilyloxy)-androst-5-en-17β-yl hemisuccinate (3)

Procedure adapted from András Keglevich; Vivian Zsiros; Péter Keglevich; Áron Szigetári; Miklós Dékány; Csaba Szántay; Erzsébet Memyák; János Wölfling and Ldszló Hazai. Synthesis and In Vitro Antitumor Effect of New Vindoline-Steroid Hybrids. Cuff. Org. Chem. 2019, 23 (8), 959-967. Succinic anhydride (0.317 g, 3.17 mmol) was added to a stirring solution of 3β-(tert-butyldimethylsilyloxy)-androst-5-en-17β-ol (0.640 g, 1.58 mmol), Et₃N (1 mL) and DMAP 0.194 g, 1.58 mmol) in anhydrous toluene (20 mL). The reaction mixture was brought to reflux and stirred for 24 hours. The reaction was diluted with CH₂Cl₂ (40 mL), water (20 mL), then treated with 2M HCl solution until pH=2. The aqueous layer was further extracted with CH₂Cl₂ (2×40 mL). The combined organic extract was washed with saturated NaCl solution, dried over anhydrous MgSO₄ and filtered. The solvent was removed under reduced pressure and the residue obtained was purified by silica column chromatography (5% AcOH:20% EtOAc:n-hexanes) to yield 3β-(tert-butyldimethylsilyloxy)-androst-5-en-17β-yl hemisuccinate (0.763 g, 95%) as a white solid.

m.p 164-168° C.; ¹H NMR (400 MHz, CDCl₃) δ 5.31 (d, J=4.3 Hz, 1H), 4.62 (t, J=8.4 Hz, 1H), 3.47 (m, 1H), 2.72-2.59 (m, 4H), 2.30-1.05 (m, 19H) 1.00 (s, 3H), 0.88 (s, 9H), 0.79 (s, 3H), 0.05 (s, 6H), COOH not observed; ¹³C NMR (101 MHz, CDCl₃) δ 177.9, 172.2, 141.8, 120.9, 83.4, 72.7, 51.2, 50.3, 42.9, 42.6, 37.5, 36.9, 36.8, 32.2, 31.9, 31.6, 29.3, 29.2, 27.6, 26.1, 23.7, 20.7, 19.6, 18.4, 12.1, −4.4 (2C); LRMS (ESI−) found m/z 503.2 [M−H]⁻; HRMS (ESI−) found m/z 503.3192 [M−H]⁻ theoretical (C₂₉H₄₇O₅Si) m/z 503.3193 [M−H]⁻; IR 3000, 2964, 1735, 1705 cm⁻¹; specific rotation [α]_{D}²⁵−10.30 (c 1.0, CHCl₃).

### 3β-(tert-Butyldimethylsilyloxy)-androst-5-en-17β-yl succinate-primaquine amide (4)

Procedure adapted from Fernandes, I.; Vale, N.; de Freitas, V.; Moreira, R.; Mateus, N.; Gomes, P. Anti-Tumoral Activity of Imidazoquines, a New Class of Antimalarials Derived from Primaquine. Bioorg. Med. Chem. Lett 2009, 19 (24), 6914-6917. EDC HCl (0.189 g, 0.992 mmol) was added to a stirring solution of 3β-(tert-butyldimethylsilyloxy)-androst-5-en-17β-yl hemisuccinate (0.250 g, 0.496 mmol), HOBt (67 mg, 0.05 mmol) and DIPEA (0.75 mL) in anhydrous CH₂Cl₂ (5 mL). Separate to this, primaquine bisphosphate (0.248 g, 0.546 mmol) was added to a solution of DIPEA (0.75 mL) in CH₂Cl₂ (5 mL) and was stirred until dissolved. After 30 minutes, the two solutions were combined, and then stirred at room temperature for 24 hours. The reaction was diluted with CH₂Cl₂ (40 mL) and 5% citric acid solution (40 mL). The aqueous layer was further extracted with CH₂Cl₂ (2×40 mL). The combined organic extract was washed with saturated NaHCO₃ solution and saturated NaCl solution, dried over anhydrous Na₂SO₄ and filtered. The solvent was removed under reduced pressure and the residue obtained was purified by silica column chromatography (3% MeOH:CH₂Cl₂) to yield 3β-(tert-butyldimethylsilyloxy)-androst-5-en-17β-yl succinate-primaquine amide (288 mg, 92%) as a green solid.

m.p 94-96° C.; ¹H NMR (400 MHz, CD₂Cl₂) δ 8.50 (d, J=4.1 Hz, 1H), 7.94 (d, J=8.2 Hz, 1H), 7.31 (dd, J=8.2, 4.2 Hz, 1H), 6.35 (d, J=2.2 Hz, 1H), 6.27 (d, J=2.2 Hz, 1H), 6.03 (d, J=8.4 Hz, 1H), 5.69 (s, 1H), 5.30 (s, 1H), 4.56 (t, J=8.4 Hz, 1H), 3.87 (s, 3H), 3.63 (s, 1H), 3.47 (tt, J=10.4, 4.7 Hz, 1H), 3.29-3.17 (m, 2H), 2.58 (t, J=6.7 Hz, 2H), 2.38 (t, J=6.8 Hz, 2H), 2.27-0.88 (m, 23H), 1.28 (d, J=6.4 Hz, 3H), 0.99 (s, 3H), 0.88 (s, 9H), 0.77 (s, 3H), 0.05 (s, 6H); ¹³C NMR (101 MHz, CD₂Cl₂) δ 173.2, 171.5, 159.9, 145.4, 144.7, 142.0, 135.7, 135.1, 130.3, 122.3, 121.1, 97.0, 92.0, 83.3, 72.9, 55.5, 51.4, 50.6, 48.2, 43.2, 42.8, 39.8, 37.7, 37.2, 37.0, 34.4, 32.5, 32.1, 31.9, 31.5, 30.2, 27.9, 26.8, 26.1, 23.9, 21.0, 20.7, 19.6, 18.4, 12.1, −4.5 (2C); LRMS (ESI+) found m/z 746.5 [M+H]⁺; HRMS (ESI+) found m/z 746.4929 [M+H]⁺, theoretical (C₄₄H₆₈N₃O₅) m/z 746.4928 [M+H]⁺; IR 3379, 3314, 2929, 2854, 1731, 1648, 1615, 1595, 1576, 1519 cm⁻¹.

### 3β-Hydroxyandrost-5-en-17β-yl succinate-primaquine amide (5; C-17-Prim; BC5B)

Procedure adapted from Yamashita, T.; Kawai, N.; Tokuyama, H.; Fukuyama, T. Stereocontrolled Total Synthesis of (−)-Eudistomin C. J. Am. Chem. Soc. 2005, 127 (43), 15038-15039. Camphor sulfonic acid (0.155 g, 6.66 mmol) was added to a stirring solution of 3β-(tert-butyldimethylsilyloxy)-androst-5-en-17β-yl succinate-primaquine amide (284 mg, 0.333 mmol) in CH₂Cl₂ (7.5 mL) and MeOH (7.5 mL). The reaction mixture was stirred at room temperature for 3 hours. The reaction was diluted with saturated NaHCO₃ solution (30 mL) and extracted with CH₂Cl₂ (20 mL). The aqueous layer was further extracted with CH₂Cl₂ (2×30 mL). The combined organic extract was washed with saturated NaCl solution, dried over anhydrous Na₂SO₄ and filtered. The solvent was removed under reduced pressure and the residue obtained was purified by silica column chromatography (3% MeOH:CH₂Cl₂) to yield 3β-hydroxyandrost-5-en-17β-yl succinate-primaquine amide (194 mg, 92%) as a green solid.

m.p 78-80° C. ¹H NMR (400 MHz, MeOD) δ 8.55-8.48 (m, 1H), 8.06 (d, J=8.2 Hz, 1H), 8.01-7.94 (m, 1H), 7.42-7.35 (m, 1H), 6.51-6.44 (m, 1H), 6.35 (s, 1H), 5.32 (d, J=4.6 Hz, 1H), 4.56-4.48 (m, 1H), 3.87 (s, 3H), 3.70-3.59 (m, 1H), 3.44-3.35 (m, 1H), 3.26-3.15 (m, 2H), 2.59 (t, J=6.0 Hz, 2H), 2.45 (t, J=6.7 Hz, 2H), 2.27-0.77 (m, 23H), 1.28 (m, 3H), 0.98/0.96 (m, 3H), 0.77/0.76 (m, 3H), OH and aniline NH not observed; ¹³C NMR (101 MHz, MeOD) δ 174.18/174.17, 174.1, 161.0, 146.2, 145.3, 142.2, 136.5, 136.3, 131.6, 122.9, 122.1, 98.35/98.33, 93.0, 84.3, 72.3, 55.7, 52.16/52.11, 51.49/51.46, 48.6, 43.6/43.59, 43.0, 40.4, 38.4, 37.94/37.93, 37.7, 35.00/34.99, 32.9, 32.4, 32.3, 31.6, 30.69/30.68, 28.4, 27.22/27.20, 24.4, 21.6, 20.7, 19.9, 12.4; LRMS (ESI+) found m/z 632.4 [M+H]⁺; HRMS found m/z 632.4061 [M+H]⁺, theoretical (C₃₈H₅₄N₃O₅) m/z 632.4063 [M+H]⁺; IR 3316, 2932, 1728, 1651, 1615, 1575, 1519 cm⁻¹.

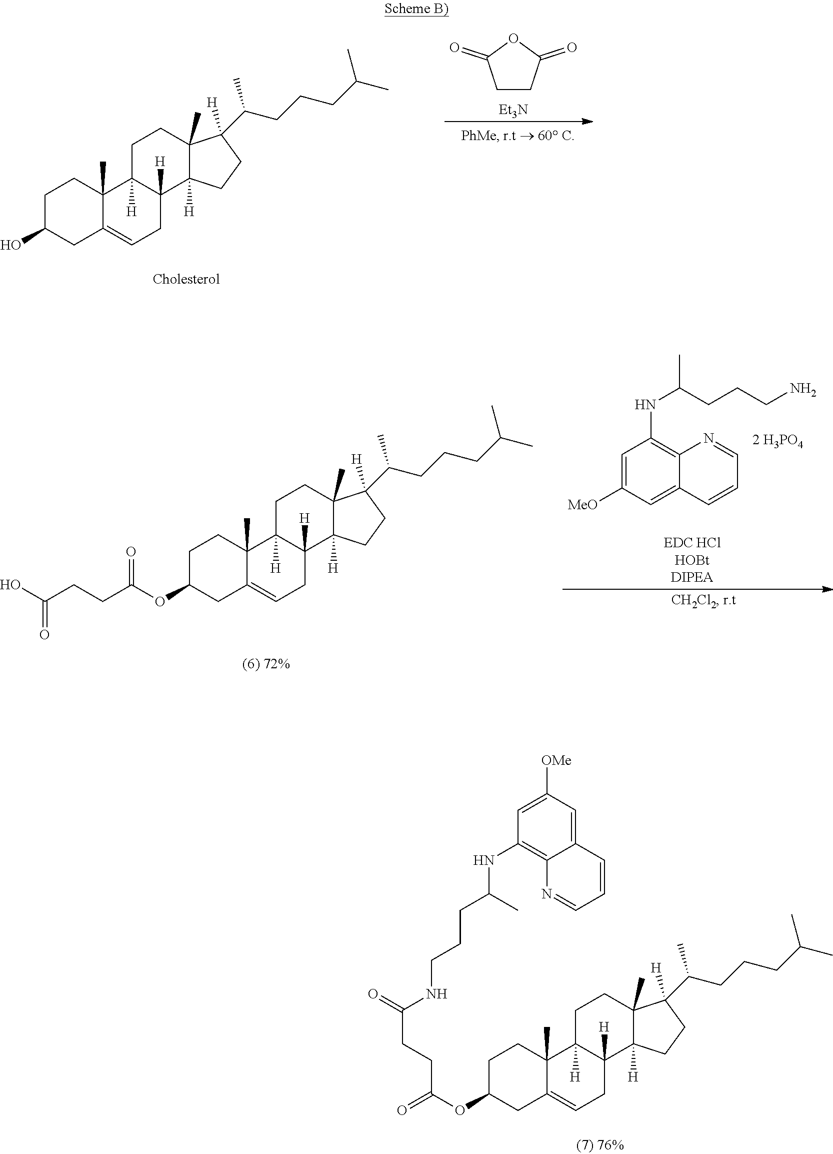

### Cholester-3-yl hemisuccinate (6)

Procedure adapted from Kumar, P.; Shankar Rao, D. S.; Krishna Prasad, S.; Jayaraman, N. In-Plane Modulated Smectic Ã vs Smectic ‘A’ Lamellar Structures in Poly(Ethyl or Propyl Ether Imine) Dendrimers. Polymer 2016, 86, 98-104. Cholesterol (773.4 mg, 2 mmol) and succinic anhydride (320.1 mg, 3.2 mmol) were dissolved in dry toluene (4 mL). Triethylamine (70 μL, 0.5 mmol) was added to the suspension and the reaction mixture was stirred at 60° C. overnight. The reaction mixture was cooled down to ambient temperature and the pale-yellow suspension was treated with water (10 mL) and the organic layer extracted with dichloromethane (2×20 mL). The combined organic extracts were washed with 2 M hydrochloric acid (3×5 mL), water (3×5 mL) and saturated brine solution (8 mL) before being dried over anhydrous (Na₂SO₄). The solution was then dried in vacuo to afford cholester-3-yl hemisuccinate (758.2 mg, 72%) as a white solid.

¹H NMR (400 MHz, CDCl₃) δ 5.37 (d, J 5.1 Hz, 1H), 4.63 (m, 1H), 2.64 (m, 4H), 2.32 (d, J 8.0 Hz, 2H), 2.03-1.76 (m, 5H), 1.62-1.02 (m, 18H), 1.00 (s, 3H), 0.98-0.92 (m, 3H), 0.89 (d, J 6.5 Hz, 3H), 0.84 (dd, J 6.6, 1.8 Hz, 6H), 0.65 (s, 3H); LRMS (ESI+) found m/z 509 [M+Na]⁺; IR (ATR, solid) 2937, 1707, 1465, 1435, 1377, 1316, 1247, 1180, 1000, 942, 799, 736, 655 cm⁻¹.

### Cholester-3b-yl succinate-primaquine amide (7, C-3-Prim)

Procedure adapted from Fernandes. I.; Vale, N.; de Freitas, V.; Moreira, R.; Mateus, N.; Gomes, P. Anti-Tumoral Activity of Imidazoquines, a New Class of Antimalarials Derived from Primaquine. Bioorg. Med. Chem. Lett. 2009, 19 (24), 6914-6917. EDC HCl (39 mg, 0.21 mmol) was added to a stirring solution of cholester-3-yl hemisuccinate (50 mg, 0.10 mmol), HOBt (21 mg, 0.15 mmol) and DIPEA (0.5 mL) in anhydrous CH₂Cl₂ (5 mL). Separate to this, primaquine bisphosphate (51 mg, 0.11 mmol) was added to a solution of DIPEA (0.5 mL) in CH₂Cl₂ (5 mL) and was stirred until dissolved. After 30 minutes, the two solutions were combined, and then stirred at room temperature for 24 hours. The reaction mixture was diluted with CH₂Cl₂ (20 mL), water (20 mL) and 5% citric acid solution until pH 5. The combined organic extract was washed with saturated NaHCO₃ solution and saturated NaCl solution, dried over anhydrous Na₂SO₄ and filtered. The solvent was removed under reduced pressure and the residue obtained was purified by silica column chromatography (3% MeOH:CH₂Cl₂) to yield cholester-3b-yl succinate-primaquine amide (57 mg, 76%) as a green oil.

¹H NMR (400 MHz, CD₂Cl₂) δ 8.54-8.47 (m, 1H), 7.97-7.90 (m, 1H), 7.31 (dd, J=8.2, 4.2 Hz, 1H), 6.35 (d, J=2.5 Hz, 1H), 6.27 (d, J=2.5 Hz, 1H), 6.02 (d, J=8.1 Hz, 1H), 5.82-5.72 (m, 1H), 5.35 (d, J=4.7 Hz, 1H), 4.60-4.49 (m, 1H), 3.87 (s, 3H), 3.67-3.57 (m, 1H), 3.31-3.15 (m, 2H), 2.56 (t, J=6.8 Hz, 2H), 2.38 (t, J=6.8 Hz, 2H), 2.29 (d, J=7.7 Hz, 2H), 1.28 (d, J=6.4 Hz, 3H), 1.00 (s, 3H), 0.92 (d, J=6.5 Hz, 3H), 0.87 (dd, J=6.6, 1.5 Hz, 6H), 0.68 (s, 3H); ¹³C NMR (101 MHz, CD₂C₂) δ 172.7, 171.6, 159.9, 145.4, 144.7, 140.2, 135.7, 135.1, 130.3, 122.9, 122.3, 97.0, 92.0, 74.6, 57.1, 56.6, 55.5, 50.8, 48.2, 42.7, 40.2, 39.9, 39.8, 38.5, 37.4, 37.0, 36.6, 36.2, 34.3, 32.3, 32.3, 31.4, 30.3, 28.6, 28.4, 28.1, 26.8, 24.6, 24.2, 23.0 (2C), 22.7, 21.4, 20.7, 19.5, 18.9, 12.0; LRMS (ESI+) found m/z 728.6 [M+H]⁺; HRMS (ESI+) found m/z 728.5364 [M+H]⁺, theoretical (C₄₆H₇₀N₃O₄) m/z 728.5366 [M+H]⁺; IR 3379, 2936, 2463, 1732, 1635, 1618, 1577, 1520 cm⁻¹.

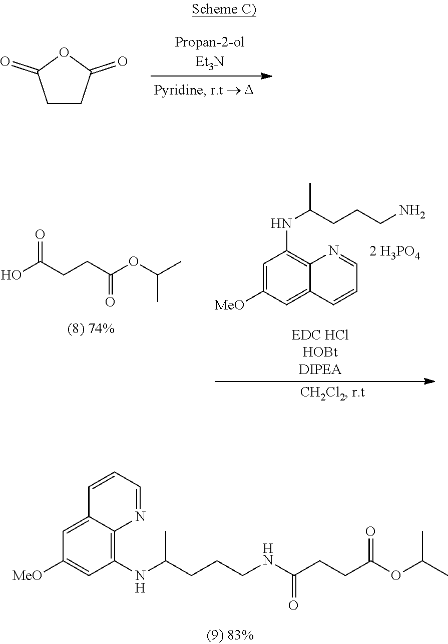 [SMILES: O=C1CCC(=O)O1 ; CC(C)OC(=O)CCC(=O)O ; COc1cc(NC(C)CCCN)c2ncccc2c1 ; COc1cc(NC(C)CCCNC(=O)CCC(=O)OC(C)C)c2ncccc2c1]

### Propan-2-yl succinate (8)

Procedure adapted from Cole, K. P.; Ryan, S. J.; Groh, J. M.; Miller, R. D. Reagent-Free Continuous Thermal Tert-Butyl Ester Deprotection. Org. Synth. Flow Med. Chem. 2017, 25 (23), 6209-6217. Succinic anhydride (2.00 g, 20.0 mmol) was added to a stirring solution of pyridine (2.0 mL, 25 mmol) in propan-2-ol (20 mL). The solution was heated to reflux and stirred for 20 h. The reaction mixture was concentrated under reduced pressure. The residue obtained was partitioned between CH₂Cl₂ (30 mL) and water (40 mL), then treated with 2M HCl solution until pH=2. The aqueous layer was further extracted with CH₂Cl₂ (2×30 mL). The combined organic extract was washed with saturated NaCl solution (60 mL), dried over anhydrous MgSO₄ and filtered. The solvent was removed under reduced pressure and the residue obtained was purified by silica column chromatography (20% EtOAc:n-hexanes) to yield propan-2-yl succinate (2.38 g, 74%) as a white oily solid.

m.p 52-53° C.; ¹H NMR (400 MHz, CDCl₃) δ 11.09 (s, 1H), 5.02 (hept, J=6.3 Hz, 1H), 2.67 (t, J=6.5 Hz, 2H), 2.58 (t, J=6.5 Hz, 2H), 1.23 (d, J=6.3 Hz, 6H); ¹³C NMR (101 MHz, CDCl₃) δ 178.3, 171.6, 68.3, 29.2, 29.0, 21.7; LRMS (ESI−) found m/z 159.1 [M−H]⁻; HRMS (ESI−) found m/z 159.0658 [M−H]⁻, theoretical (C₇H₁₁O₄) m/z 159.0657 [M−H]⁻; IR 3124, 1726, 1713 cm⁻¹.

### Propan-2-yl succinate-primaquine amide (9, Prim-link)

Procedure adapted from Fernandes, I.; Vale, N.; de Freitas, V.; Moreira, R.; Mateus, N.; Gomes, P. Anti-Tumoral Activity of Imidazoquines, a New Class of Antimalarials Derived from Primaquine. Bioorg. Med. Chem. Left. 2009, 19 (24). 6914-6917. EDC HCl (0.191 g, 1 mmol) was added to a stirring solution of propan-2-yl succinate (80 mg, 0.50 mmol), HOBt (68 mg, 0.5 mmol) and DIPEA (0.65 mL) in anhydrous CH₂Cl₂ (5 mL). Separate to this, primaquine bisphosphate (0.250 g, 0.550 mmol) was added to a solution of DIPEA (0.65 mL) in CH₂Cl₂ (5 mL) and was stirred until dissolved. After 30 minutes, the two solutions were combined, and then stirred at room temperature for 24 hours. The reaction mixture was diluted with CH₂Cl₂ (40 mL) and 5% citric acid solution (30 mL). The aqueous layer was further extracted with CH₂Cl₂ (2×40 mL). The combined organic extract was washed with saturated NaHCO₃ solution and saturated NaCl solution, dried over anhydrous Na₂SO₄ and filtered. The solvent was removed under reduced pressure and the residue obtained was purified by silica column chromatography (3% MeOH:CH₂Cl₂) to yield propan-2-yl succinate-primaquine amide (167 mg, 83%) as a green oil.

¹H NMR (400 MHz, MeOD) δ 8.48 (d, J=4.2 Hz, 1H), 8.01 (d, J=8.2 Hz, 1H), 7.37-7.32 (m, 1H), 6.47-6.41 (m, 1H), 6.33-6.28 (m, 1H), 4.94 (dq, J=12.5, 6.3 Hz, 1H), 3.86 (s, 3H), 3.69-3.59 (m, 1H), 3.23-3.13 (m, 2H), 2.54 (t, J=6.8 Hz, 2H), 2.43 (t, J=6.9 Hz, 2H), 1.74-1.58 (m, 4H), 1.28 (d, J=6.3 Hz, 3H), 1.18 (d, J=6.3 Hz, 6H); ¹³C NMR (101 MHz, MeOD) δ 174.3, 173.8, 161.0, 146.2, 145.3, 136.5, 136.3, 131.6, 122.9, 98.3, 93.0, 69.2, 55.6, 48.9, 40.3, 34.9, 31.5, 30.8, 27.1, 22.0, 20.8; LRMS (ESI+) found m/z 424.2 [M+Na]⁺; HRMS (ESI+) found m/z 402.2387 [M+H]⁺, theoretical (C₂₂H₃₂N₃O₄) m/z 402.2393 [M+H]⁺; IR 3390, 2981, 2863, 2472, 2414, 1727, 1634, 1615, 1594, 1576, 1518 cm⁻¹.

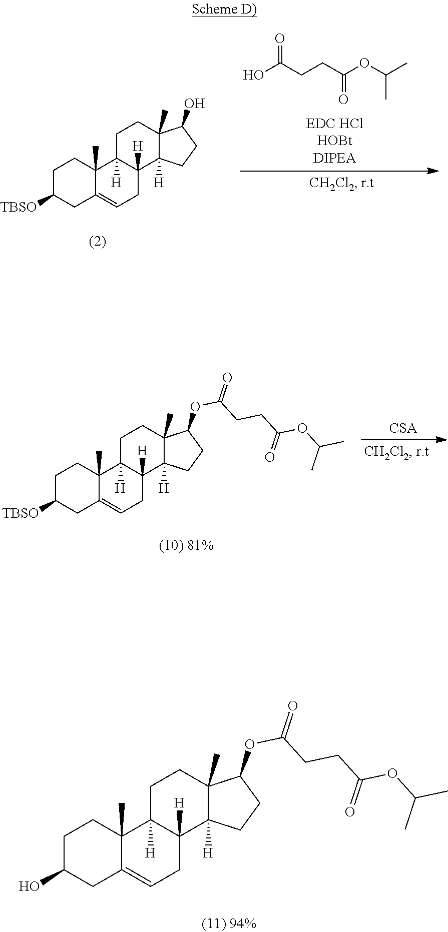

### 3β-(tert-Butyldimethylsilyloxy)-androst-5-en-17β-yl succinate-propan-2-yl diester (10)

Procedure adapted from Krausova, B.; Slavikova, B.; Nekardova, M.; Hubalkova, P.; Vyklicky, V.; Chodounska, H.; Vyklicky, L.; Kudova, E. Positive Modulators of the N-Methyl-d-Aspartate Receptor: Structure-Activity Relationship Study of Steroidal 3-Hemiesters. J. Med. Chem. 2018, 61 (10), 4505-4516. EDC HCl (0.335 g, 1.74 mmol) was added to a stirring solution of propan-2-yl succinate (0.279 g, 1.74 mmol), DMAP (0.213 g, 1.74 mmol) and DIPEA (0.5 mL) in CH₂Cl₂ (15 mL). After 15 minutes 3β-(tert-butyldimethylsilyloxy)-androst-5-en-17β-ol (0.353 g, 0.874 mmol) was added to the solution. The reaction mixture was stirred at room temperature for 21 hours. The reaction was diluted with CH₂Cl₂ (40 mL) and 5% citric acid solution (50 mL). The aqueous layer was further extracted with CH₂Cl₂ (2×40 mL). The combined organic extract was washed with saturated NaCl solution, dried over anhydrous Na₂SO₄ and filtered. The solvent was removed under reduced pressure and the residue obtained was purified by silica column chromatography (5% EtOAc:n-hexanes) to yield 3β-(tert-butyldimethylsilyloxy)-androst-5-en-17β-yl succinate-propan-2-yl diester (348 mg, 81%) as a white solid.

m.p 105-107° C.; ¹H NMR (400 MHz, CDCl₃) δ5.31 (d, J=4.9 Hz, 1H), 5.01 (hept, J=6.3 Hz, 1H), 4.61 (t, J=8.4 Hz, 1H), 3.47 (tt, J=10.7, 4.7 Hz, 1H), 2.65-2.54 (m, J=4.4 Hz, 4H), 2.30-0.91 (m, 19H), 1.23 (d, J=6.3 Hz, 6H), 1.00 (s, 3H), 0.88 (s, 9H), 0.80 (s, 3H), 0.05 (s, 6H); ¹³C NMR (101 MHz, CDCl₃) δ; 172.4, 171.9, 141.8, 120.9, 83.2, 72.7, 68.2, 51.2, 50.3, 42.9, 42.6, 37.5, 36.9, 36.8, 32.2, 31.9, 31.6, 29.8, 29.6, 27.6, 26.1, 23.7, 22.0 (2C), 20.7, 19.6, 18.4, 12.1, −4.4 (2C); LRMS (ESI+) found m/z 547.4 [M+H]⁺; HRMS (ESI+) found m/z 569.3638 [M+Na]⁺, theoretical (C₃₂H₅₄O₅SiNa) m/z 569.3638 [M+Na]⁺; IR 2938, 1729 cm⁻¹; specific rotation [α]_{D}²⁵−44.00 (c 1.0, CHCl₃).

### 3β-Hydroxyandrost-5-en-17β-yl succinate-propan-2-yl diester (11, C-17-link)

Procedure adapted from Yamashita, T.; Kawai, N.; Tokuyama, H.; Fukuyama, T. Stereocontrolled Total Synthesis of (−)-Eudistomin C. J. Am. Chem. Soc. 2005, 127 (43), 15038-15039. Camphor sulfonic acid (0.302 g, 1.30 mmol) was added to a stirring solution of 3β-(tert-butyldimethylsilyloxy)-androst-5-en-17β-yl succinate-propan-2-yl diester (0.360 g, 0.650 mmol) in CH₂Cl₂ (10 mL) and MeOH (10 mL). The reaction mixture was stirred at room temperature for 25 minutes. The reaction was diluted with CH₂Cl₂ (50 mL) and saturated NaHCO₃ solution (50 mL). The aqueous layer was further extracted with CH₂Cl₂ (2×50 mL). The combined organic extract was washed with saturated NaCl solution, dried over anhydrous Na₂SO₄ and filtered. The solvent was removed under reduced pressure and the residue obtained was purified by silica column chromatography (20% EtOAc:n-hexanes) to yield 3β-hydroxyandrost-5-en-17β-yl succinate-propan-2-yl diester (265 mg, 94%) as a white solid.

m.p 105-107° C.; ¹H NMR (400 MHz, CDCl₃) δ 5.34 (d, J=5.4 Hz, 1H), 5.01 (hept, J=6.3 Hz, 1H), 4.66-4.57 (m, 1H), 3.52 (tt, J=11.1, 4.5 Hz, 1H), 2.63-2.55 (m, 4H), 2.32-0.91 (m, 21H), 1.23 (d, J=6.3 Hz, 6H), 1.01 (s, 3H), 0.80 (s, 3H), OH not observed; ¹³C NMR (101 MHz, CDCl₃) δ 172.4, 171.9, 141.0, 121.4, 83.2, 71.8, 68.2, 51.2, 50.2, 42.6, 42.4, 37.4, 36.9, 36.7, 31.8, 31.7, 31.6, 29.7, 29.6, 27.6, 23.7, 21.9 (2C), 20.7, 19.6, 12.1; LRMS (ESI+) found m/z 433.3 [M+H]⁺; HRMS (ESI+) found m/z 455.2773 [M+Na]⁺, theoretical (C₂₆H₄₀O₅Na) m/z 455.2773 [M+Na]⁺; IR 3443, 2933, 1730 cm⁻¹; specific rotation [α]_{D}²⁵−38.80 (c 1.0, CHCl₃).

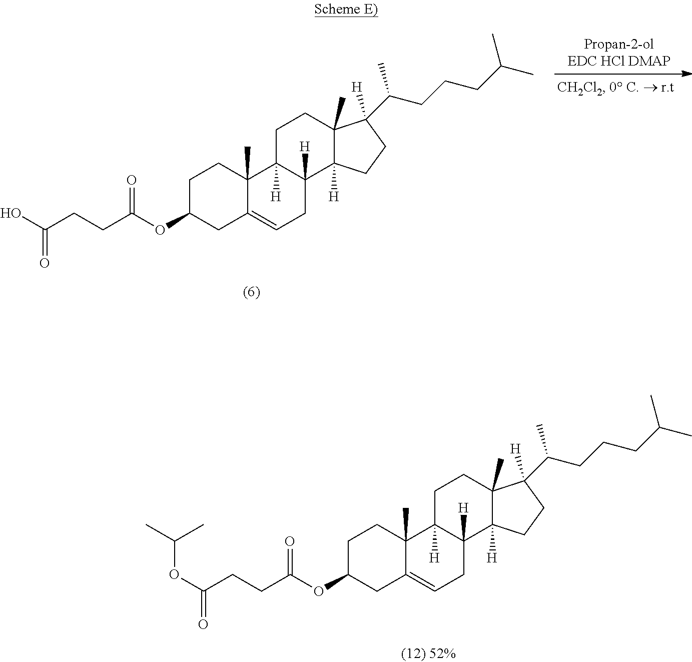

### 3β-Succinate-(propan-2-yl diester)-cholest-5-ene (12, C-3-Link)

A solution of cholesterol hemisuccinate (20.1 mg, 0.041 mmol) and EDC·HCl (15.8 mg, 0.082 mmol) in dry dichloromethane (1 mL) was stirred at 0° C. for 5 minutes under N₂. To the solution, DMAP (9.9 mg, 0.082 mmol), isopropanol (6.3 μL, 0.082 mmol) and additional dichloromethane (1 mL) were added. The reaction mixture was warmed up to ambient temperature and left to stir under N₂ overnight. The solution was then quenched by dropwise addition of 5% citric acid solution (5 mL). The pale-yellow solution was then extracted with ethyl acetate (3×5 mL) to give a colourless organic extract. The combined organic extracts were then washed with saturated brine solution (10 mL) and dried over anhydrous MgSO₄. The crude material was purified by flash chromatography (silica, 1:3 ethyl acetate:hexanes) to afford 3β-succinate-(propan-2-yl diester)-cholest-5-ene (11.4 mg, 52%) as a white solid.

¹H NMR (400 MHz, CDCl₃) δ 5.35 (d, J 4.05 Hz, 1H), 4.99 (sept., J 6.3 Hz, 1H), 4.60 (m, 1H), 2.56 (s, 4H), 2.29 (d, J 7.8 Hz, 2H), 2.02-1.76 (m, 5H), 1.61-1.25 (m, 12H), 1.21 (d, J 6.3 Hz, 6H), 1.17-1.02 (m, 7H), 0.99 (s, 3H), 0.96-0.91 (m, 2H), 0.89 (d, J 6.5 Hz, 3H), 0.84 (dd, J 6.6, 1.8 Hz, 6H), 0.65 (s, 3H); ¹³C NMR (175 MHz, CDCl₃) δ 172.1, 171.9, 139.8, 122.9, 74.5, 68.2, 56.9, 56.4, 50.2, 42.5, 39.9, 39.7, 38.3, 37.2, 36.8, 36.4, 36.0, 32.1, 32.1, 29.8, 29.8, 28.4, 28.2, 28.0, 24.5, 24.0, 23.0, 22.8, 22.0, 21.2, 19.5, 18.9, 12.1; LRMS (ESI+) found m/z 551 [M+Na]⁺; HRMS (ESI+) found m/z 529.4266 [M+H]⁺, theoretical (C₃₄H₅₇O₄) m/z 529.4251 [M+H]⁺; IR (ATR, solid) 2941, 2890, 2869, 2852, 1728, 1467, 1373, 1318, 1309, 1164, 1107, 996, 982, 958 cm⁻¹; specific rotation [α]_{D}²⁵−24.78 (c 1.0, CHCl₃).

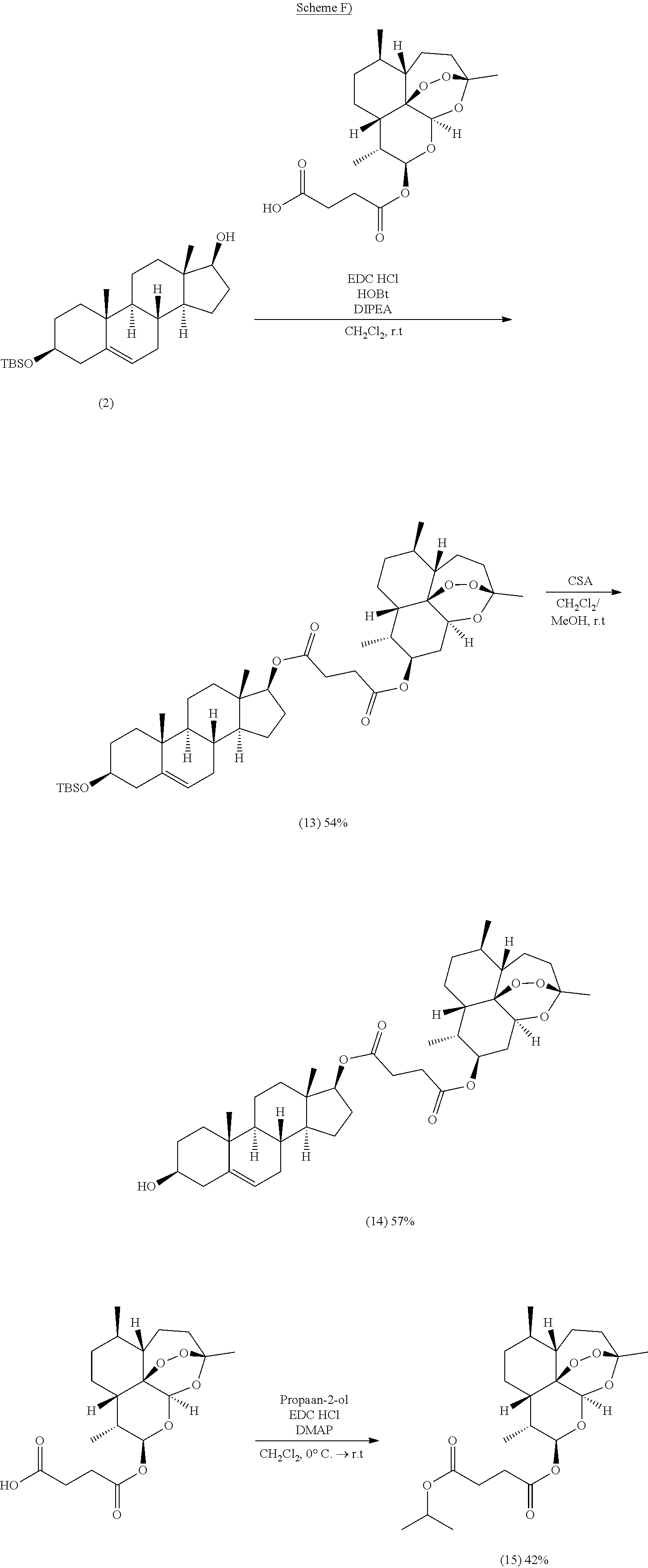

### 3β-(tert-Butyldimethylsilyloxy)-androst-5-en-17β-yl succinate-artesunate ester (13)

A solution of artesunate (10.9 mg, 0.028 mmol) and EDC·HCl (10 mg, 0.052 mmol) was stirred in dichloromethane (2 mL) in a flame dried round bottom flask under N₂. After 5 m a solution of DMAP (6.4 mg, 0.052 mmol) and 3β-(tert-butyldimethylsilyloxy)-androst-5-en-17β-ol (21.1 mg, 0.052 mmol) in dichloromethane (2 mL) was added. The combined solution was left stirring at room temperature for 20 h before being quenched with 5% citric acid solution (5 mL) and extracted with EtOAc (4×5 mL). The combined organic layers were washed with brine (10 mL) and dried over anhydrous MgSO₄ and finally concentrated under reduced pressure to give a crude white solid. The crude product was purified by flash chromatography (silica, 10% EtOAc in hexanes) to give 3β-(tert-butyldimethylsilyloxy)-androst-5-en-17β-yl succinate-artesunate ester (11.9 mg, 54%) as a clear oil.

¹H NMR (600 MHz, CDCl₃) δ 5.79 (d, J 9.9 Hz, 1H), 5.43 (s, 1H), 5.31 (d, J 5.3 Hz, 1H), 4.61 (dd, J 8.3, 8.9 Hz, 1H), 3.48 (m, 1H), 2.72 (m, 2H), 2.65 (m, 2H), 2.57 (m, 1H), 2.37 (ddd, J 4.66, 14.26, 13.62 Hz, 1H), 2.26 (m, 1H), 2.01 (m, 2H), 1.89 (m, 1H), 1.75 (m, 5H), 1.63 (m, 2H), 1.51 (m, 7H), 1.43 (s, 3H), 1.39 (m, 2H), 1.30 (m, 3H), 1.16 (m, 1H), 1.04 (m, 2H), 1.00 (s, 3H), 0.96 (d, J 6.20 Hz, 3H), 0.92 (m, 1H), 0.88 (s, 9H), 0.85 (d, J 7.20 Hz, 3H), 0.79 (s, 3H), 0.05 (s, 6H); ¹³C NMR (150 MHz, CDCl₃) δ 172.20, 171.30, 141.79, 120.89, 104.60, 92.24, 91.64, 83.28, 80.27, 51.72, 51.22, 50.29, 45.39, 42.95, 37.52, 37.43, 36.93, 36.79, 36.37, 34.24, 32.20, 31.94, 31.86, 31.64, 29.50, 29.28, 27.64, 26.12, 26.09, 24.73, 23.74, 22.15, 20.67, 20.37, 19.61, 18.42, 12.26, 12.16, −4.44; LRMS (ESI+) found m/z 794 [M+Na]⁺; HRMS (ESI+) found m/z 793.4686 [M+Na]⁺, theoretical (C₄₄H₇₀O₉SiNa) m/z 793.4687 [M+Na]⁺; specific rotation [α]_{D}²⁵−14.36 (c 0.8, CHCl₃).

### 3β-Hydroxyandrost-5-en-17β-yl succinate-artesunate ester (14, C-17-Art)

A solution of 3β-(tert-butyldimethylsilyloxy)-androst-5-en-17β-yl succinate-artesunate ester (30 mg, 0.039 mmol) in dry tetrahydrofuran (10 mL) was treated with 1M TBAF solution in THF (140 μL, 0.14 mmol) and stirred for 40 h at room temperature. The solvent was removed under reduced pressure to give a crude yellow oil. The crude material was purified by flash chromatography (silica, 1:1 ethyl acetate:hexanes) to afford 3β-hydroxyandrost-5-en-17β-yl succinate-artesunate ester (14.8 mg, 57%) as a colourless oil.

¹H NMR (400 MHz, CDCl₃) δ 5.79 (d, J 9.8 Hz, 1H), 5.43 (s, 1H), 5.34 (d, J 5.0 Hz, 1H), 4.61 (t, J 8.4 Hz, 1H) 3.52 (td, J 5.4, 11.2 Hz, 1H), 2.72-0.88 (m, 36H) 1.43, (s, 3H), 1.01 (s, 3H), 0.96 (d, J 6.2 Hz, 3H), 0.85 (d, J 7.2 Hz, 3H), 0.80 (s, 3H); ¹³C NMR (175 MHz, CDCl₃) δ 172.20, 171.30, 141.01, 121.43, 104.60, 92.26, 91.64, 83.27, 80.27, 71.85, 60.54, 51.73, 51.20, 50.21, 45.40, 42.60, 42.40, 37.43, 36.92, 36.71, 36.37, 34.25, 31.94, 31.86, 31.77, 31.60, 29.50, 29.28, 27.64, 26.10, 24.73, 22.15, 20.69, 20.36, 19.56, 14.34, 12.24, 12.16; LRMS (ESI+) found m/z 679 [M+Na]⁺; HRMS (ESI+) found m/z 679.3815 [M+Na]⁺, theoretical (C₃₈H₅₆O₉Na) m/z 679.3822 [M+Na]⁺; IR (ATR, solid) 3450, 2930, 1735, 1454, 1375, 1158, 1037, 1015, 877 cm⁻¹.

### Propan-2-yl artesunate ester (15, Art-Link)

A solution of artesunate (20 mg, 0.052 mmol) and EDC·HCl (19.9 mg, 0.104 mmol) was stirred in dry dichloromethane (1 mL) at 0° C. for 5 minutes under N₂. To the solution. DMAP (12.7 mg, 0.104 mmol), isopropanol (7.78 μL, 0.104 mmol) and additional dichloromethane (1 mL) were added. The reaction mixture was warmed up to ambient temperature and left to stir under N₂ for 4 h. Additional isopropanol (7.78 μL, 0.054 mmol) was added and the reaction was stirred for another 2 h. The solution was then quenched by dropwise addition of 5% citric acid solution (5 mL). The colourless solution was then extracted with ethyl acetate (3×5 mL) to give a colourless organic extract. The extract was then washed with saturated brine solution (10 mL) and dried over anhydrous MgSO₄. The crude material was purified by flash chromatography (silica, 1:3 ethyl acetate:hexanes) to afford propan-2-yl artesunate ester (9.4 mg, 42%) as a white solid.

¹H NMR (400 MHz, CDCl₃) δ 5.77 (d, J 9.9 Hz, 1H), 5.41 (s, 1H), 4.99 (sept., J 6.2 Hz, 1H), 2.71-2.50 (m, 5H), 2.35 (td, J 14.0, 4.0 Hz, 1H), 2.00 (ddd, J=14.6, 4.7, 3.1 Hz, 1H), 1.81 (m, 1H), 1.72 (m, 2H), 1.62-1.57 (m, 1H), 1.51-1.43 (m, 1H), 1.40 (s, 3H), 1.37-1.23 (m, 3H), 1.20 (dd, J 6.3, 0.8 Hz, 6H), 1.06-0.96 (m, 1H), 0.94 (d, J 5.9 Hz, 3H), 0.83 (d, J 7.1 Hz, 3H); ¹³C NMR (175 MHz, CDCl₃) δ 171.8, 171.4, 104.7, 92.3, 91.7, 80.3, 68.3, 51.8, 45.5, 37.5, 36.5, 34.3, 32.0, 29.5, 29.5, 26.2, 24.8, 22.2, 22.0, 20.4, 12.3; LRMS (ESI+) found m/z 449 [M+Na]⁺; HRMS (ESI+) found m/z 449.2149 [M+Na]⁺, theoretical (C₂₂H₃₄O₈Na) m/z 449.2146 [M+Na]⁺; IR (ATR, solid) 2976, 2928, 2875, 1750, 1730, 1454, 1375, 1201, 1159, 1102, 1036, 1012, 926, 876, 825 cm⁻¹; specific rotation [α]_{D}²⁵+11.18 (c 1.0, CHCl₃).

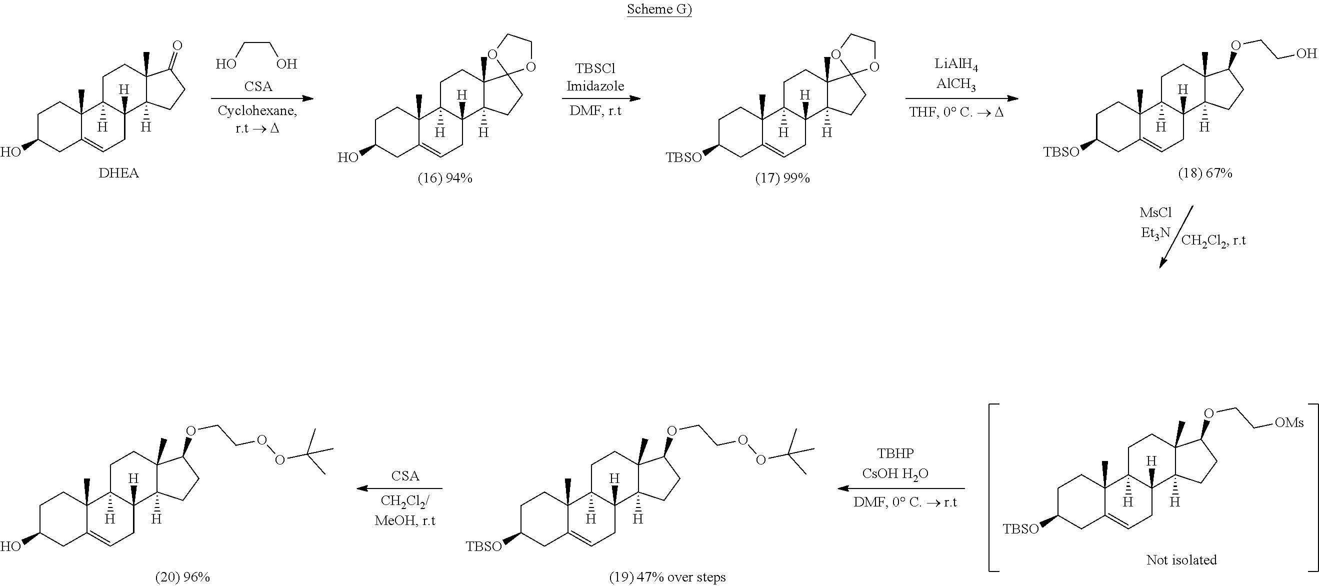

### 3β-Hydroxy-17,17-(ethylenedioxy)-androst-5-ene (16)

Procedure adapted from Calogeropoulou, T.; Avlonitis, N.; Minas, V.; Alexi, X.; Pantzou, A.; Charalampopoulos, I.; Zervou. M.; Vergou, V.; Katsanou, E. S.; Lazaridis, I.; Alexis, M. N.; Gravanis, A. Novel Dehydroepiandrosterone Derivatives with Antiapoptotic, Neuroprotective Activity. J. Med. Chem. 2009, 52 (21), 6569-6587. 3β-Hydroxyandrost-5-en-17-one (1.0 g, 3.5 mmol) was combined with ethylene glycol (0.60 mL, 10 mmol) and camphor sulfonic acid (10 mg, 0.14 mmol) in cyclohexane (100 mL). The reaction flask was fitted with a Dean-Stark apparatus, stirred, and brought to reflux for 4 hours. The reaction was cooled, diluted with EtOAc (50 mL) and poured into saturated NaHCO₃ solution (50 mL). The aqueous layer was further extracted with EtOAc (2×50 mL). The combined organic extract was washed with saturated NaCl solution, dried over anhydrous Na₂SO₄ and filtered. The solvent was removed under reduced pressure to yield 3β-hydroxy-17,17-(ethylenedioxy)-androst-5-ene (1.08 g, 94%) as a white solid.

m.p 162-165° C. (lit. 162-165° C.: Calogeropoulou, T.; Avionitis, N.; Minas, V.; Alexi, X.; Pantzou, A.; Charalampopoulos, I.; Zervou, M.; Vergou, V.; Katsanou, E. S.; Lazaridis, I.; Alexis, M. N.; Gravanis, A. Novel Dehydroepiandrosterone Derivatives with Antiapoptotic, Neuroprotective Activity. J. Med. Chem. 2009, 52 (21), 6569-6587); ¹H NMR (400 MHz, CDCl₃) δ 5.37-5.31 (m, 1H), 3.95-3.83 (m, 4H), 3.52 (m, 1H), 2.31-0.93 (m, 19H), 1.00 (s, 3H), 0.85 (s, 3H), OH not observed; ¹³C NMR (101 MHz, DMSO-de) δ 141.3, 120.2, 118.5, 69.9, 64.6, 64.0, 50.2, 49.7, 45.1, 42.2, 36.9, 36.1, 33.7, 31.7, 31.4, 30.8, 30.2, 22.3, 20.1, 19.2, 14.1; LRMS (ESI+) found m/z 355.2 [M+Na]⁺; HRMS (ESI+) found m/z 355.2259 [M+Na]⁺, theoretical (C₂₁H₃₂O₃Na) m/z 355.2249 [M+Na]⁺; IR 3546, 3140, 2989, 2828 cm⁻¹

### 3β-(tert-Butyldimethylsilyloxy)-17,17-(ethylenedioxy)-androst-5-ene (17)

Procedure adapted from Yamauchi, T.; Kato, M.; Mikami, T.; Fujimura, Y. Synthesis of 1-Deoxymaxacalcitol. Heterocycles 2005, 65 (9), 2111. tert-Butyldimethylsilyl chloride (0.580 g, 3.84 mmol) was added to a stirring solution of 3β-hydroxy-17,17-(ethylenedioxy)-androst-5-ene (0.850 g, 2.56 mmol) and imidazole (0.436 g, 6.40 mmol) in anhydrous DMF (10 mL). The reaction mixture was stirred at room temperature for 2 hours. The reaction was diluted with EtOAc (50 mL) and water (50 mL). The aqueous layer was further extracted with EtOAc (2×50 mL). The combined organic extract was washed with saturated NaCl solution, dried over anhydrous MgSO₄ and filtered. The solvent was removed under reduced pressure to yield 3β-(tert-butyldimethylsilyloxy)-17,17-(ethylenedixoy)-androst-5-ene (1.125 g, 99%) as a white solid.

m.p 119-121° C. (lit. 121-122° C.: Shen, Y.; Burgoyne, D. L. Efficient Synthesis of IPL576,092: A Novel Anti-Asthma Agent. J. Org. Chem. 2002, 67 (11), 3908-3910); ¹H NMR (400 MHz, CDCl₃) δ 5.34-5.28 (m, 1H), 3.96-3.84 (m, 4H), 3.48 (m, 1H), 2.29-0.92 (m, 19H), 1.00 (s, 3H), 0.88 (s, 9H), 0.85 (s, 3H), 0.05 (s, 6H); ¹³C NMR (101 MHz, Dioxane) 6142.23, 121.66, 119.94, 73.29, 65.67, 65.07, 51.47, 51.13, 46.46, 43.60, 38.07, 37.40, 34.81, 33.02, 32.88, 32.08, 31.43, 26.30, 23.46, 21.26, 19.70, 18.74, 14.62, −4.36 (2C); LRMS (ESI+) found m/z 469.3 [M+Na]⁺, HRMS (ESI+) found m/z 469.3118 [M+Na]⁺, theoretical (C₂₇H₄₆O₃SiNa) m/z 469.3114 [M+Na]⁺; IR 2987, 2886 cm⁻¹.

### 3β-(tert-Butyldimethylsilyloxy)-17β-(2-hydroxyethoxy)-androst-5-ene (18)

Procedure adapted from Upasani, R. B.; Fick, D. B.; Hogenkamp, D. J.; Lan, N. C. Neuroactive Steroids of the Androstane and Pregnane Series. U.S. Pat. No. 5,925,630A, Jul. 20, 1999, 3β-(tert-Butyldimethylsilyloxy)-17,17-(ethylenedioxy)-androst-5-ene (1.00 g, 2.24 mmol) in anhydrous THF (12 mL) was cooled over an ice bath under N₂ atmosphere. AlCl₃ (0.600 g, 4.48 mmol) in anhydrous THF (5 mL) and LiAlH₄ (0.170 g. 4.48 mmol) in anhydrous THF (5 mL) were added individually by dropwise addition to the previous solution with stirring. The reaction was brought to reflux and stirred for 2 hours. The reaction was then cooled over an ice bath and quenched by addition of 10% Rochelle's salt solution (10 mL) and EtOAc (15 mL). The mixture was left stirring for a further 20 minutes. The solution was then diluted with EtOAc (50 mL) and 10% Rochelle's salt (50 mL). The aqueous layer was further extracted with EtOAc (2×50 mL). The combined organic extract was washed with saturated NaCl solution, dried over anhydrous MgSO₄ and filtered. The solvent was removed under reduced pressure and the residue obtained was purified by silica column chromatography (15% EtOAc:n-hexanes) to yield 3β-(tert-butyldimethylsilyloxy)-17β-(2-hydroxyethoxy)-androst-5-ene (0.676 g, 67%) as a white solid.

m.p 117-119° C.; ¹H NMR (400 MHz, CDCl₃) δ 5.30 (m, 1H), 3.71-3.66 (m, 2H), 3.61-3.44 (m, 3H), 3.35 (t, J=8.3 Hz, 1H), 2.29-0.91 (m, 19H), 1.00 (s, 3H), 0.88 (s, 9H), 0.77 (s, 3H), 0.05 (s, 6H), OH not observed; ¹³C NMR (101 MHz, CDCl₃) δ 141.8, 121.0, 89.6, 72.7, 71.1, 62.2, 51.8, 50.4, 43.0, 42.9, 38.0, 37.5, 36.8, 32.2, 31.9, 31.7, 28.2, 26.1, 23.5, 20.9, 19.6, 18.4, 11.7, −4.4 (2C); LRMS (ESI+) found m/z 471.3 [M+Na]⁺; HRMS (ESI+) found m/z 471.3284 [M+Na]⁺, theoretical (C₂₇H₄₈O₃SiNa) m/z 471.3270 [M+Na]⁺; IR 3462, 2927, 2856 cm⁻1; specific rotation [α]_{D}²⁵−33.20 (c 1.0, CHCl₃).

### 17β-(2-(tert-Butylperoxy)-ethoxy)-3β-(tert-butyldimethylsilyloxy)-androst-5-ene (19)

Procedure adapted from Dussault, P. H.; Lee, H.-J.; Liu, X. Selectivity in Lewis Acid-Mediated Fragmentations of Peroxides and Ozonides: Application to the Synthesis of Alkenes, Homoallyl Ethers, and 1,2-Dioxolanes. J. Chem. Soc. Perkin 1 2000, No. 17, 3006-3013. Methane sulfonyl chloride (180 mL, 2.27 mmol) was added to a cooled and stirring solution of 3β-(tert-butyldimethylsilyloxy)-17β-(2-hydroxyethoxy)-androst-5-ene (300 mg, 0.670 mmol) and Et₃N (340 mL, 2.44 mol) in CH₂Cl₂. The reaction was brought to room temperature and stirred under N₂ for 45 minutes. The solvent was removed under reduced pressure and yellow residue obtained was redissolved in anhydrous DMF (30 mL) and added to a cooled solution of CsOH monohydrate (500 mg, 3.35 mmol) and tert-butyl hydroperoxide (˜240 mL, 1.2 mmol) (nominally 5.0 M in decane) in anhydrous DMF (12 mL). The reaction was brought to room temperature and stirred under N₂ for 24 hours. A further amount of CsOH monohydrate (430 mg. 2.86 mmol) was added producing a strongly yellow solution which was stirred at room temperature for a further 24 hours. The reaction mixture was diluted with EtOAc (50 mL) and water (50 mL). The aqueous layer was further extracted with EtOAc (2×50 mL). The combined organic extract was washed saturated NaCl solution, dried over anhydrous Na₂SO₄ and filtered. The solvent was removed under reduced pressure and the residue obtained was purified by silica column chromatography (100% hexane, followed by 10% EtOAc:n-hexanes) to yield 17β-(2-(tert-butylperoxy)-ethoxy)-3β-(tert-butyldimethylsilyloxy)-androst-5-ene (198 mg, 57%) as a white solid.

m.p 122-124° C.; ¹H NMR (400 MHz, CDCl₃) δ 5.30 (d, J=4.6 Hz, 1H), 4.05 (t, J=5.1 Hz, 2H), 3.74-3.57 (m, 2H), 3.47 (tt, J=10.5, 4.6 Hz, 1H), 3.33 (t, J=8.2 Hz, 1H), 2.33-2.21 (m, 1H), 2.21-2.11 (m, 1H), 2.05-1.89 (m, 3H), 1.86-1.77 (m, 1H), 1.74-1.68 (m, 1H), 1.63-0.84 (m, 12H), 1.24 (s, 9H), 1.00 (s, 3H), 0.88 (s, 9H), 0.77 (s, 3H), 0.05 (s, 6H); ¹³C NMR (101 MHz, CDCl₃) δ 141.8, 121.0, 89.6, 80.4, 74.8, 72.7, 67.5, 51.8, 50.5, 43.0 (2C), 38.0, 37.6, 36.8, 32.2, 31.9, 31.7, 28.1, 26.5, 26.1, 23.5, 20.9, 19.6, 18.4, 11.6, −4.4 (2C); LRMS (ESI+) found m/z 543.4 [M+Na]⁺; HRMS (ESI+) found m/z 543.3848 [M+Na]⁺, theoretical (C₃₁H₅₆O₄SiNa) m/z 543.3846 [M+Na]⁺; specific rotation [α]_{D}²⁵+256.60 (c 1.0, CHCl₃).

### 17β-(2-(tert-Butylperoxy)-ethoxy)-androst-5-en-3β-ol (20; C-17-perox; BC86D)

Procedure adapted from Yamashita, T.; Kawai, N.; Tokuyama, H.; Fukuyama, T. Stereocontrolled Total Synthesis of (−)-Eudistomin C. J. Am. Chem. Soc. 2005, 127 (43), 15038-15039. Camphor sulfonic acid (106 mg, 0.456 mmol) was added to a stirring solution of 17β-(2-(tert-butylperoxy)-ethoxy)-3β-(tert-butyldimethylsilyloxy)-androst-5-ene (120 mg, 0.228 mmol) in CH₂Cl₂ (5 mL) and MeOH (5 mL). The reaction was stirred at room temperature for 1 hour. The reaction mixture was diluted with CH₂Cl₂ (50 mL) and saturated NaHCO₃ solution (50 mL). The aqueous layer was further extracted with CH₂Cl₂ (2×40 mL). The combined organic extract was washed with saturated NaCl solution, dried over anhydrous Na₂SO₄ and filtered. The solvent was removed under reduced pressure and the residue obtained was purified by silica column chromatography (20% EtOAc:n-hexanes) to yield 17β-(2-(tert-butylperoxy)-ethoxy)-androst-5-en-3β-ol (89 mg, 96%) as a white oily solid.

¹H NMR (400 MHz, CDCl₃) δ 5.34 (d, J=5.0 Hz, 1H), 4.05 (t, J=5.1 Hz, 2H), 3.73-3.58 (m, 2H), 3.52 (tt, J=10.9, 4.7 Hz, 1H), 3.34 (t, J=8.2 Hz, 1H), 2.35-2.18 (m, 2H), 2.07-1.90 (m, 3H), 1.88-1.79 (m, 2H), 1.62-0.88 (m, 12H), 1.25 (s, 9H), 1.01 (s, 3H), 0.78 (s, 3H), OH not observed; ¹³C NMR (101 MHz, CDCl₃) δ 141.0, 121.6, 89.6, 80.4, 74.8, 71.9, 67.5, 51.7, 50.4, 43.0, 42.4, 38.0, 37.4, 36.7, 31.9, 31.8, 31.6, 28.1, 26.5, 23.5, 20.9, 19.6, 11.6; LRMS (ESI+) found m/z 429.3 [M+Na]⁺; HRMS (ESI+) found m/z 429.2969 [M+Na]⁺, theoretical (C₂₅H₄₂O₄Na) m/z 429.2975 [M+Na]⁺; IR 3362 cm⁻¹; specific rotation [α]_{D}²⁵−49.50 (c 1.0, CHCl₃).

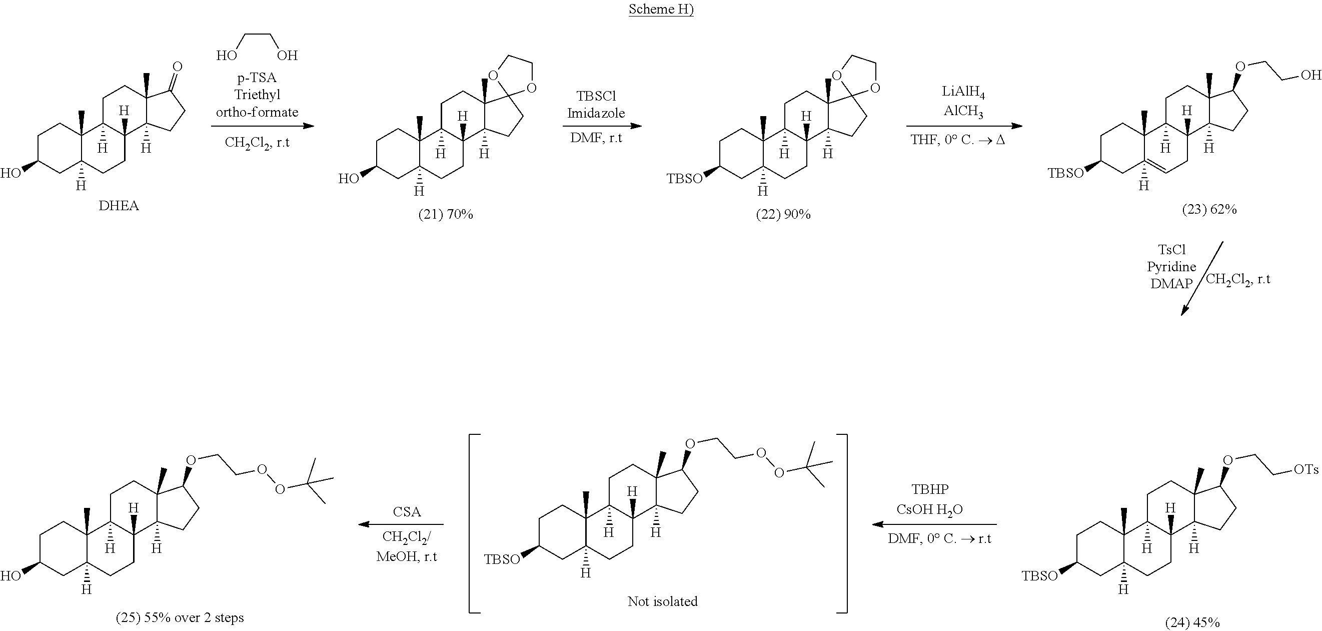

### 3β-Hydroxy-17,17-(ethylenedioxy)-5α-androstane (21)

Procedure adapted from Liu, D.; Stuhmiller, L. M.; McMorrs, T. C. Synthesis of 15β-Hydroxy-24-Oxocholesterol and 15β,29-Dihydroxy-7-Oxofucosterol. J. Chem. Soc. Perkin 1 1988, No. 8, 2161-2167. 3β-Hydroxy-5α-androstan-17-one (1.50 g, 5.16 mmol) was combined with ethylene glycol (6.41 g, 103 mmol), triethylorthoformate (4.59 g, 30.9 mmol) and p-toluenesulfonic acid (25 mg, 0.13 mmol) in CH₂Cl₂ (4 mL). The reaction was stirred under N₂ at room temperature overnight. The reaction was quenched with Et₃N (30 μL) and diluted with water (30 mL) and CH₂Cl₂ (30 mL). The aqueous layer was further extracted with CH₂Cl₂ (2×30 mL). The combined organic extract was washed with saturated NaCl solution, dried over anhydrous MgSO₄ and filtered. The solvent was removed under reduced pressure and the residue obtained was washed with cold MeOH (10 mL) and purified by silica column chromatography (30% EtOAc:n-hexanes) to yield 3β-hydroxy-17,17-(ethylenedioxy)-5α-androstane (1.20 g, 70%), as a white solid.

m.p 152-154° C. (lit. 152-155° C.: Kratena, N.; Biedermann, N.; Stojanovic, B.; Göschl, L.; Weil, M.; Enev, V. S.; Gmeiner, G.; Gärtner, P. Synthesis of a Human Long-Term Oxymetholone Metabolite. Steroids 2019, 150, 108430); ¹H NMR (400 MHz, CDCl₃) δ 3.95-3.80 (m, 4H), 3.58 (m, 1H), 1.98-0.85 (m, 21H), 0.82 (s, 3H), 0.79 (s, 3H), 0.70-0.62 (m, 1H), OH not observed; ¹³C NMR (101 MHz, Dioxane-d₈) δ 119.9, 71.0, 65.7, 65.1, 55.2, 51.1, 46.7, 45.8, 39.4, 38.0, 36.7, 36.4, 34.8, 32.5, 32.3, 31.5, 29.5, 23.4, 21.4, 14.8, 12.7. LRMS (ESI+) found m/z 335.5 [M+H]⁺; HRMS (ESI+) found m/z 335.2583 [M+H]⁺, theoretical (C₂₁H₃₅O₃) m/z 355.2586 [M+H]⁺; IR 3369, 3100, 2950, 2859 cm⁻¹.

### 3β-(tert-Butyldimethylsilyloxy)-17,17-(ethylenedioxy)-5α-androstane (22)

Procedure adapted from Yamauchi, T.; Kato, M.; Mikami, T.; Fujimura, Y. Synthesis of 1-Deoxymaxacalcitol. Heterocycles 2005, 65 (9), 2111. tert-Butyldimethylsilyl chloride (343 mg, 2.27 mmol) was added to a stirring solution of 3β-hydroxy-17,17-(ethylenedioxy)-5α-androstane (500 mg, 1.50 mmol) and imidazole (244 mg, 3.58 mmol) in anhydrous DMF (10 mL). The reaction was stirred at room temperature for 1 hour. The reaction mixture was diluted with water (30 mL) and extracted with ethyl acetate (3×20 mL). The combined organic extract was washed with saturated NaCl solution, dried over anhydrous MgSO₄ and filtered. The solvent was removed under reduced pressure and the residue obtained was purified by silica column chromatography (30% EtOAc:n-hexanes) to yield 3β-(tert-butyldimethylsilyloxy)-17,17-(ethylenedioxy)-5α-androstane (607 mg, 90%) as a white solid.

m.p 137-139° C.; ¹H NMR (400 MHz, Dioxane-de) δ 3.85-3.71 (m, 4H), 3.58 (m, 1H), 1.92-0.90 (m, 21H), 0.86 (s, 9H), 0.81 (s, 3H), 0.79 (s, 3H), 0.63 (m, 1H), 0.03 (s, 6H); ¹³C NMR (101 MHz, Dioxane-d₈) δ 119.9, 72.7, 65.7, 65.1, 55.2, 51.1, 46.7, 45.8, 39.6, 37.9, 36.6, 36.3, 34.8, 32.8, 32.3, 31.5, 29.4, 26.3, 23.4, 21.4, 18.7, 14.8, 12.6, −4.3 (2C); LRMS (ESI+) found m/z 449.3 [M+H]⁺; HRMS (ESI+) found m/z 471.3289 [M+Na]⁺, theoretical (C₂₇H₄₈O₃SiNa) m/z 471.3270: IR 2929, 2857 cm⁻; specific rotation [α]_{D}⁵²−14.70 (c 1.0, CHCl₃).

### 3β-(tert-Butyldimethylsilyloxy)-17β-(2-hydroxyethoxy)-5α-androstane (23)

Procedure adapted from Upasani, R. B.; Fick, D. B.; Hogenkamp, D. J.; Lan, N. C. Neuroactive Steroids of the Androstane and Pregnane Series. U.S. Pat. No. 5,925,630A, Jul. 20, 1999. 3β-(tert-Butyldimethylsilyloxy)-17,17-(ethylenedioxy)-5α-androstane (500 mg, 1.12 mmol) in anhydrous THF (5 mL) was cooled over an ice bath under N₂ atmosphere. AlCl₃ (300 mg, 2.24 mmol) in anhydrous THF (1.5 mL) and LiAlH₄ (85 mg, 2.24 mmol) in anhydrous THF (2 mL) were added individually by dropwise addition to the previous solution with stirring. The reaction was brought to reflux and stirred for 2 hours. The reaction was then cooled over an ice bath and quenched by addition of 10% Rochelle's salt solution (11 mL) and EtOAc (15 mL). The mixture was left stirring for a further 30 minutes. The solution was then diluted with water (20 mL) and the aqueous layer was extracted with EtOAc (3×20 mL). The combined organic extract was washed with saturated NaCl solution, dried over anhydrous MgSO₄ and filtered. The solvent was removed under reduced pressure and the residue obtained was purified by silica column chromatography (30% EtOAc:n-hexanes) to yield 3β-(tert-butyldimethylsilyloxy)-17β-(2-hydroxyethoxy)-5α-androstane (312 mg, 62%) as a white solid.

m.p 127-129° C.; ¹H NMR (400 MHz, CDCl₃) δ 3.73-3.63 (m, 2H), 3.62-3.50 (m, 3H), 3.33 (t, J=8.2 Hz, 1H), 2.30-0.80 (m, 21H), 0.88 (s, 9H), 0.80 (s, 3H), 0.75 (s, 3H), 0.59 (m, 1H), 0.04 (s, 6H), OH not observed; ¹³C NMR (101 MHz, CDCl₃) δ 89.7, 72.3, 71.0, 62.3, 54.7, 51.5, 45.2, 43.3, 38.8, 38.2, 37.4, 35.7, 35.5, 32.1, 31.8, 28.8, 28.2, 26.1, 23.5, 21.0, 18.4, 12.5, 11.9, −4.4 (2C); LRMS (ESI+) m/z 473.4 [M+Na]⁺; HRMS (ESI+) m/z 473.3433 [M+Na]⁺, theoretical (C₂₇H₅₀O₃SiNa) m/z 473.3427 [M+Na]⁺; IR 3464, 2936, 2918, 2855 cm⁻¹; specific rotation [α]_{D}²⁵+6.30 (c 1.0, CHCl₃).

### 2-(3β-(tert-Butyldimethylsilyloxy)-5α-androstan-17β-yloxy)-ethyl para-toluenesulfonate (24)

Procedure adapted from Hatakeyama, S.; Yoshino, M.; Eto, K.; Takahashi, K.; Ishihara, J.; Ono, Y.; Saito, H.; Kubodera, N. Synthesis and Preliminary Biological Evaluation of 20-Epi-Eldecalcitol [20-Epi-1α,25-Dihydroxy-2β-(3-Hydroxypropoxy)Vitamin D3: 20-Epi-ED-71]. Proc. 14th Vitam. Workshop 2010, 121 (1), 25-28. 3β-(tert-Butyldimethylsilyloxy)-17β-(2-hydroxyethoxy)-5α-androstane (68 mg, 0.15 mmol), pyridine (120 μL, 1.5 mmol), DMAP (19 mg, 0.16 mmol) and tosyl chloride (30 mg, 0.16 mmol) in CH₂Cl₂ (5 mL) were stirred at room temperature overnight. The reaction was poured over water (20 mL), brought to pH 2 with 2M HCl solution and extracted with CH₂Cl₂ (3×15 mL). The combined organic extract was washed with saturated NaCl solution, dried over anhydrous MgSO₄ and filtered. The solvent was removed under reduced pressure and the residue obtained was purified by silica column chromatography (10% EtOAc:n-hexanes) to yield 2-(3β-(tert-butyldimethylsilyloxy)-5α-androstan-17β-yloxy)-ethyl para-toluenesulfonate (41 mg, 45%) as a white oily solid.

¹H NMR (400 MHz, CDCl₃) δ 7.79 (d, J=8.3 Hz, 2H), 7.33 (d, J=8.0 Hz, 2H), 4.11 (t, J=5.0 Hz, 2H), 3.68-3.58 (m, 2H), 3.57-3.50 (m, 1H), 3.24 (t, J=8.3 Hz, 1H), 2.44 (s, 3H), 1.94-0.83 (m, 21H), 0.88 (s, 9H), 0.79 (s, 3H), 0.65 (s, 3H), 0.60-0.55 (m, 1H), 0.04 (s, 6H); ¹³C NMR (101 MHz, CDCl₃) δ 144.8, 133.3, 129.9, 128.1, 89.9, 72.3, 69.8, 67.5, 54.7, 51.3, 45.2, 43.2, 38.8, 38.0, 37.4, 35.7, 35.4, 32.1, 31.8, 28.8, 28.0, 26.1, 23.4, 21.8, 21.0, 18.4, 12.5, 11.7, −4.4 (2C): LRMS (ESI+) found m/z 627.4 [M+Na]⁺; HRMS (ESI+) found m/z 627.3517 [M+Na]⁺, theoretical (C₃₄H₅₆O₅SSiNa) m/z 627.3515 [M+Na]⁺; IR 2928, 2855, 1598, 1363 cm⁻¹

### 17α-(2-(tert-Butylperoxy)-ethoxy)-5α-androstan-3β-ol (25)

Procedure adapted from Dussault, P. H.; Lee, H.-J.; Liu, X. Selectivity in Lewis Acid-Mediated Fragmentations of Peroxides and Ozonides: Application to the Synthesis of Alkenes, Homoallyl Ethers, and 1,2-Dioxolanes. J. Chem. Soc. Perkin 1 2000, No. 17, 3006-3013. CsOH monohydrate (30 mg, 0.18 mmol) in anhydrous DMF (1 mL) under N₂ was cooled over an ice bath. tert-Butyl hydroperoxide (˜5.9 mg, ˜0.065 mmol) (nominally 5.5M in decane) was added dropwise and stirred for 30 minutes. 2-(3β-(tert-Butyldimethylsilyloxy)-5α-androstan-17β-yloxy)-ethyl para-toluenesulfonate (22 mg, 0.036 mmol) was added to the stirring solution. The reaction was brought to room temperature and stirred under N₂ overnight. The reaction mixture was diluted with water (20 mL) and extracted with CH₂Cl₂ (3×20 mL). The combined organic extract was washed saturated NaCl solution, dried over anhydrous Na₂SO₄ and filtered. The solvent was removed under reduced pressure and the residue obtained was purified by silica column chromatography (100% hexane, followed by 10% EtOAc:n-hexanes) to yield 17β-(2-(tert-butylperoxy)-ethoxy)-3β-(tert-butyldimethylsilyloxy)-5α-androstane (12 mg, 0.023 mmol) which was used without further purification. 17β-(2-(tert-Butylperoxy)-ethoxy)-3β-(tert-butyldimethylsilyloxy)-5α-androstane and CSA (1 mg, 0.4 μmol) in MeOH/CH₂Cl₂ (2 mL) were treated a per general procedure E for 2 hours. The reaction was quenched with saturated NaHCO₃ and diluted with water (5 mL). The aqueous layer was extracted with CH₂Cl₂ (2×15 mL). The combined organic extract was washed with saturated NaCl solution, dried over anhydrous Na₂SO₄ and filtered. The solvent was removed under reduced pressure and the residue obtained was purified by silica column chromatography (5% EtOAc:n-hexanes) to yield 17β-(2-(tert-butylperoxy)-ethoxy)-5α-androstan-3β-ol (8.1 mg, 55% over 2 steps) as a clear oil.

¹H NMR (400 MHz, CDCl₃) δ 4.04 (t, J=5.1 Hz, 2H), 3.68-3.55 (m, 3H), 3.32 (t, J=8.2 Hz, 1H), 2.02-0.83 (m, 21H), 1.24 (s, 9H), 0.81 (s, 3H), 0.74 (s, 3H), 0.65-0.58 (m, 1H) OH not observed; ¹³C NMR (101 MHz, CDCl₃) δ 89.7, 80.4, 74.8, 71.5, 67.4, 54.6, 51.4, 45.1, 43.3, 38.4, 38.2, 37.2, 35.7, 35.5, 31.8, 31.7, 28.8, 28.2, 26.5, 23.5, 21.1, 12.5, 11.8; LRMS (ESI+) found m/z 431.4 [M+Na]⁺, HRMS (ESI+) m/z 431.3148 [M+Na]⁺, theoretical (C₂₅H₄₄O₄Na) m/z 431.3137 [M+Na]⁺. IR 3332, 2973, 2927, 2855 cm⁻¹

### Example 4: Assessment of Efficacy of Conjugate with Metronidazole Against P. falciparum

This example shows that coupling of a known anti-parasitic compound, metronidazole, to steroid increases its efficacy against P. falciparum.

The metronidazole-steroid conjugate, referred to as BC62D, is shown below:

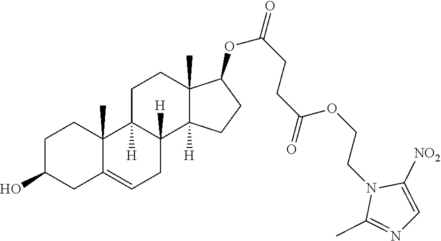

Metronidazole is an anti-parasitic compound that is used for the treatment of parasitic disease like giardiasis, trichomoniasis, amoebiasis and dracunculiasis, but does not show any significant efficacy against the malaria parasite Plasmodium spp.

Metronidazole was coupled to C17 of steroid as described above, and the resulting compound was compared to metronidazole alone.

FIG. 20A shows the structures of: (i) metronidazole alone (unmodified), (ii) metronidazole coupled to a steroid (via a linker at C17) (BC62D), and (iii) metronidazole coupled to a linker (BC61D).

FIG. 20B shows dose response curves of metronidazole alone, metronidazole coupled to a steroid (via a linker) (BC62D), and metronidazole coupled to a linker (BC61D), showing the effect of these compounds on asexual P. falciparum stages. A control compound (metronidazole coupled to a linker) was used to show specificity.

As can be seen from FIG. 20B, BC62D (steroid-coupled metronidazole) exhibits a lower IC₅₀ than metronidazole alone. Metronidazole alone showed almost no effect in the range tested. BC62D killed P. falciparum asexual stages with an IC50 of 24 μM. The control compound (metronidazole and linker; BC61D) does not show the same enhanced activity as BC62D (steroid-coupled metronidazole).

Hence, steroid-coupled metronidazole showed significantly increased efficacy against asexual P. falciparum stages compared to metronidazole alone.

### Example 5: Assessment of Efficacy of Conjugate with Hydroxychloroquine in Plasmodium Asexual Stages

This example shows that coupling of a known anti-parasitic compound, hydroxychloroquine, to a steroid overcomes drug resistance in Plasmodium asexual stages.

The hydroxychloroquine-steroid conjugate, referred to as BC75D, can be prepared using methods similar to those used to prepare other steroid conjugates as described above. The structure of BC75D is shown below:

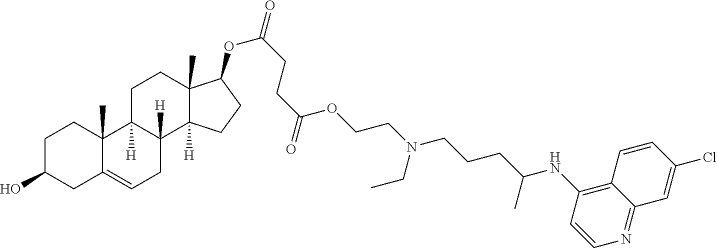

Chloroquine was a major synthetic drug used to treat malaria until the emergence of drug resistant P. falciparum strains limited its clinical use. Resistance is, at least in part, conferred through a mutant P. falciparum chloroquine resistance transporter (PfCRT) protein which transports chloroquine away from its active site in the digestive vacuole (Chinappi, M., A. Via, P. Marcatili, and A. Tramontano. 2010. ‘On the mechanism of chloroquine resistance in Plasmodium falciparum’, PLoS One. 5: e14064: Martin. R. E., R. V. Marchetti, A. I. Cowan, S. M. Howitt, S. Broer, and K. Kirk. 2009. ‘Chloroquine transport via the malaria parasite's chloroquine resistance transporter’, Science, 325: 1680-2).

The inventors compared the efficacy of different compounds against the chloroquine sensitive P. falciparum strains 3D7 and C2_GC03 against the chloroquine resistant P. falciparum strain C4_Dd2 (Sidhu, A. B., D. Verdier-Pinard, and D. A. Fidock. 2002. ‘Chloroquine resistance in Plasmodium falciparum malaria parasites conferred by pfcrt mutations’, Science, 298: 210-3).

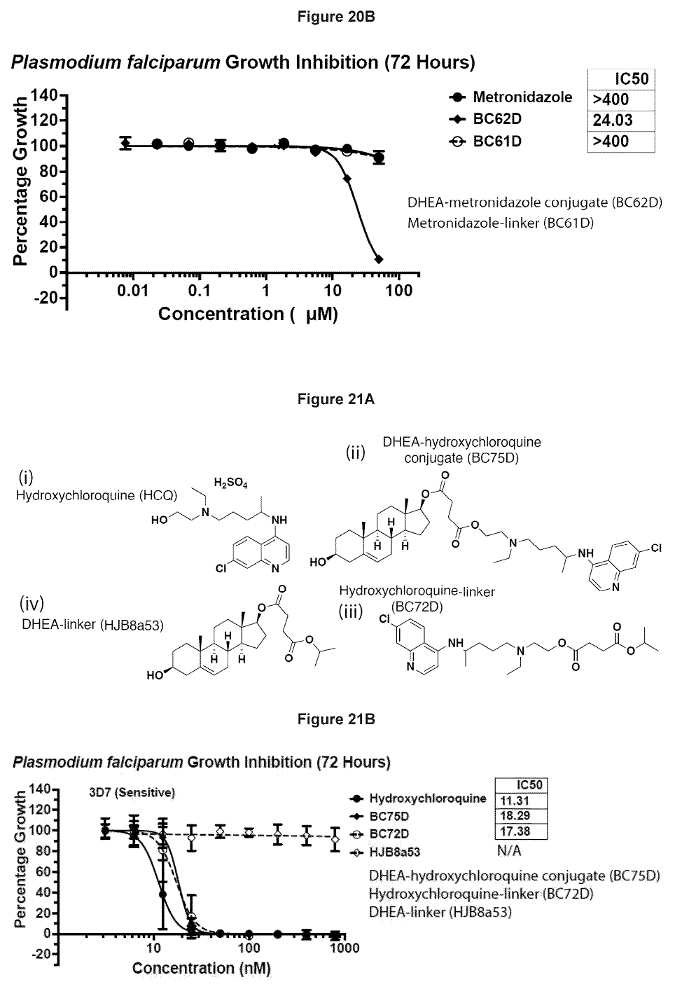

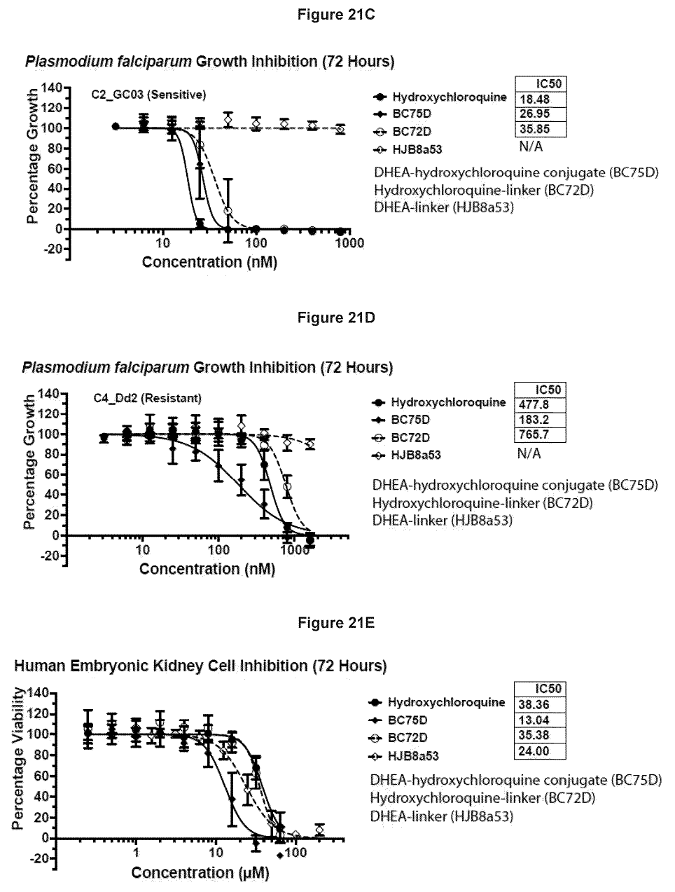

FIG. 21A shows the structure of hydroxychloroquine alone (i), hydroxychloroquine coupled to steroid (via a linker at C17) (BC75D) (ii), hydroxychloroquine coupled to a linker (BC72D) (iii), and steroid & linker (HJB8a53) (iv).

FIG. 21B shows dose response curves of hydroxychloroquine alone and hydroxychloroquine coupled to steroid (via a linker), showing the effect of these compounds on asexual P. falciparum stages of the chloroquine sensitive strain 3D7. Control compounds (hydroxychloroquine coupled to a linker, and a steroid coupled to a linker) were used to show specificity.

FIG. 21C shows dose response curves of hydroxychloroquine alone and hydroxychloroquine coupled to steroid (via a linker), showing the effect of these compounds on asexual P. falciparum stages of the chloroquine sensitive strain C2_GC03. This strain as been genetically modified to convert the chloroquine-resistant P. falciparum strain Dd2 to become chloroquine sensitive. Control compounds (hydroxychloroquine coupled to a linker, and a steroid coupled to a linker) were used to show specificity.

FIG. 21D shows dose response curves of hydroxychloroquine alone and hydroxychloroquine coupled to steroid (via a linker), showing the effect of these compounds on asexual P. falciparum stages of the chloroquine resistant strain C4_Dd2. Control compounds (hydroxychloroquine coupled to a linker, and a steroid coupled to a linker) were used to show specificity.

The inventors also tested for cytotoxicity against HEK293 cells (FIG. 21E), finding an approximately 3-fold difference in the IC₅₀ of hydroxychloroquine coupled to steroid compared to hydroxychloroquine alone (13 μM vs 38.36 μM), indicating lower cytotoxicity against host cells of the hydroxychloroquine-steroid conjugate compared to free hydroxychloroquine.

Although hydroxychloroquine displays a slightly better efficacy against chloroquine sensitive P. falciparum strains compared to hydroxychloroquine-steroid conjugates (1.4-1.6-fold), a much higher concentration hydroxychloroquine compared to hydroxychloroquine-steroid conjugate was needed to kill chloroquine-resistant parasites (2.6-fold). Hence, the drug-steroid conjugate was 2.6 times more effective at killing resistant parasites. At the same time, the hydroxychloroquine-steroid conjugate was 3-times less toxic to host cells than the free drug.

### Example 6: Assessment of Efficacy of Conjugates Against Leishmania tarentolae

Protozoan parasites of the genus Leishmania (non-apicomplexan parasites) cause significant disease in vertebrates, in particular in canids, rodents and humans. According to the World Health Organisation, between 700,000 and 1 million new cases occurs in humans each year. Leishmania tarentolae is a pathogen of lizards and can be used as an in vitro model system.

Activity of BC5B Against Leishmania tarentolae

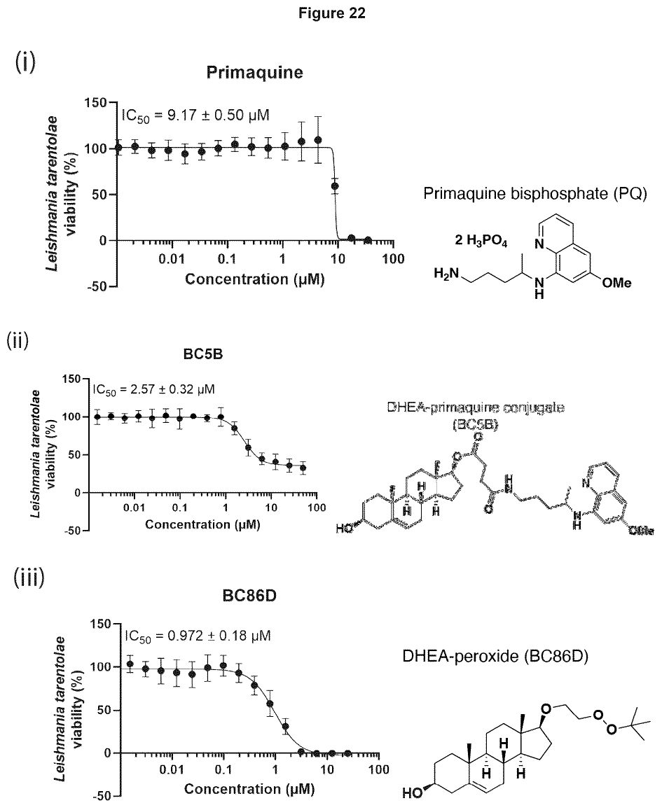

Leishmania spp. is less susceptible to primaquine than Plasmodium (FIG. 22(i)) and hence primaquine is not used for the treatment of leishmaniasis.

The ability of the steroid-primaquine conjugate (BC5B) to inhibit Leishmania tarentolae growth was assessed over 72 hours. The results are shown in FIG. 22(ii). The IC₅₀ values of steroid-coupled primaquine (BC5B), and primaquine alone were determined via a dose-response curve (FIGS. 22(i) and (ii)). The steroid-primaquine conjugate (BCSB) inhibited the growth of Leishmania tarentolae with higher efficacy than primaquine.

Activity of BC86D Against Leishmania tarentolae

Since the presumed inhibitory action of primaquine is the generation of free radicals in the cell, a steroid was conjugated to a peroxide (BC86D) and tested for its effect against Leishmania tarentolae over 72 hours.

BC86D has the following structure:

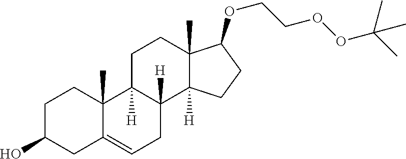

The results achieved are shown in FIG. 22(iii). The IC₅₀ of the steroid-bound peroxide (BC86D) was found to be less than 1 μM, demonstrating that the steroid-bound peroxide was effective against Leishmania tarentolae.

Also described herein are the following items:
1. A compound of Formula (I):

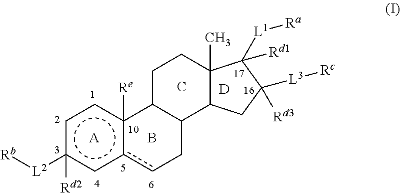

wherein:
each of R^{a}, R^{b} and R^{c} is independently selected from —H, —OH, ═O, substituted or unsubstituted —C₁₋₁₀ alkyl, substituted or unsubstituted —C₂₋₁₀ alkenyl, substituted or unsubstituted —C₂₋₁₀ alkynyl, substituted or unsubstituted —C₃₋₁₀ cycloalkyl, substituted or unsubstituted —C₅₋₁₄ cycloalkenyl, substituted or unsubstituted —C₅₋₁₄ cycloalkynyl, and an anti-parasite moiety; wherein at least one of R^{a}, R^{b} and R^{c} is an anti-parasite moiety;
provided that: when R^{a} is —OH, L¹ is absent; when R^{a} is ═O, L¹ and R^{d1} are absent; when R^{b} is —OH, L² is absent; when R^{b} is ═O, L² is absent, R^{d2} is absent, and the C2-C3 bond and the C3-C4 bond are single bonds; when R^{c} is —OH, L³ is absent; when R^{c} is ═O. L³ and R^{d3} are absent;
when R^{a}, R^{b} or R^{c} is substituted, R^{a}, R^{b} or R^{c} is substituted with one or more groups selected from Substituent Group A;
R^{d1}, when present, is H or is a group that, together with R^{a}, forms a substituted or unsubstituted ozonide ring or a substituted or unsubstituted tetraoxane ring, wherein the optional substituent may be a —C₃₋₁₀ cycloalkyl; provided that when R^{d1} and R^{a} form a substituted or unsubstituted ozonide ring or a substituted or unsubstituted tetraoxane ring, L¹ is absent; wherein, when the ozonide or tetraoxane ring is substituted, the ozonide or tetraoxane ring is substituted with one or more —C₃₋₁₀ cycloalkyl groups;
R^{d2}, when present, is H; provided that when R² is present, the C2-C3 bond and the C3-C4 bond are single bonds;
R^{d3}, when present, is H or is a group that, together with R^{c}, forms a substituted or unsubstituted ozonide ring or a substituted or unsubstituted tetraoxane ring, wherein the optional substituent may be a —C₃₋₁₀ cycloalkyl; provided that when R^{d3} and R^{c} form a substituted or unsubstituted ozonide ring or a substituted or unsubstituted tetraoxane ring, L³ is absent; wherein, when the ozonide or tetraoxane ring is substituted, the ozonide or tetraoxane ring is substituted with one or more —C₃₋₁₀ cycloalkyl groups:
R^{e} is H or CH₃, or R^{e} is absent;
when present, L¹ is a group that provides a covalent linkage between R^{a} and the C17 of ring D;
when present, L² is a group that provides a covalent linkage between R^{b} and the C3 of ring A;
when present, L³ is a group that provides a covalent linkage between R^{c} and the C16 of ring D;
ring A may be a saturated or unsaturated ring, or ring A may be an aromatic ring, provided that when ring A is an aromatic ring, the C5-C6 bond is a single bond, R^{d2} is absent, R^{e} is absent, and R^{b} is not ═O;
----- is a single bond or a double bond; provided that when the C4-C5 or C5-C10 bond is a double bond, the C5-C6 bond is a single bond; when the C5-C6 bond is a double bond, the C4-C5 and C5-C10 bonds are single bonds; and when the C3 forms a double bond with C2 or C4, R^{b} is not ═O;
or a stereoisomer thereof, or a salt thereof.
2. A compound according to item 1, wherein Formula (I) is selected from:

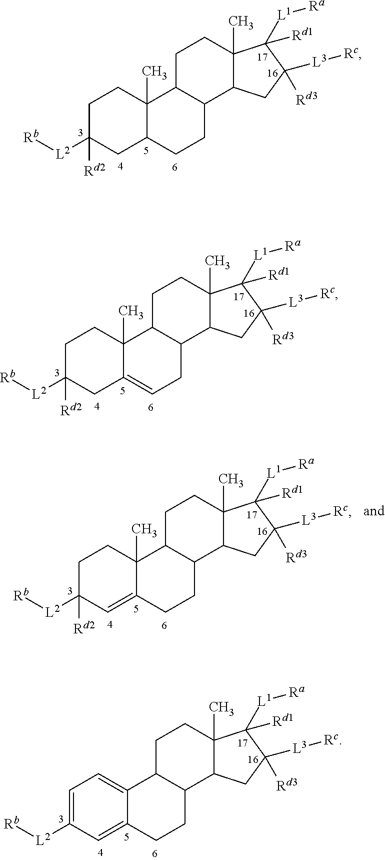

3. A compound according to item 1 or 2, wherein Formula (I) is selected from:

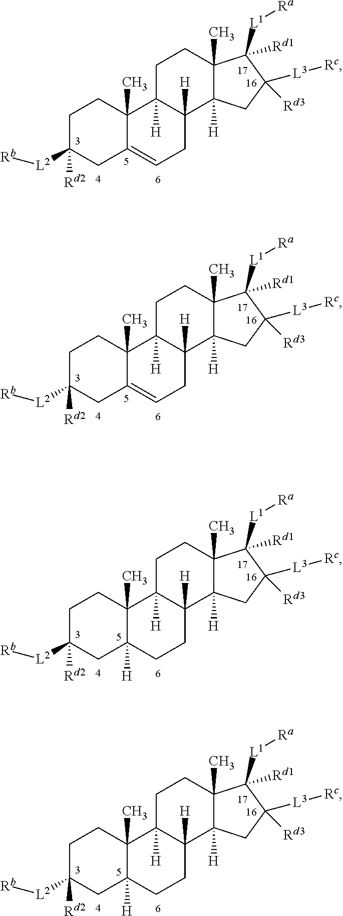

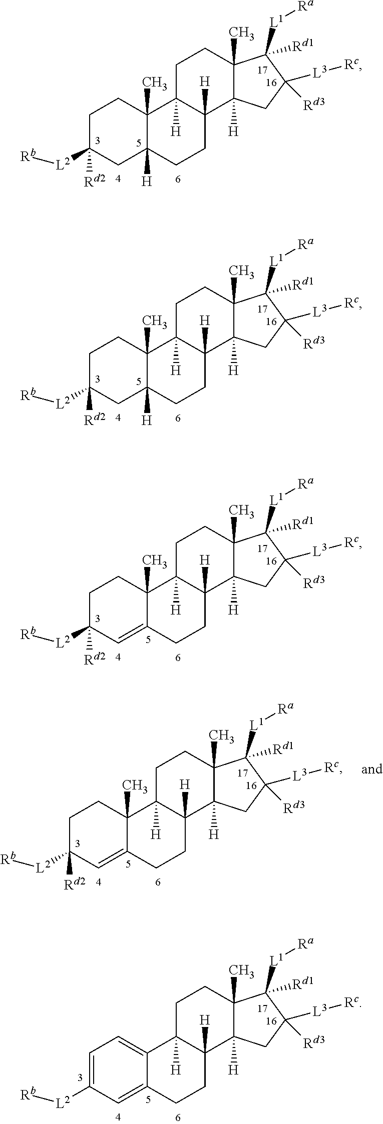

4. A compound according to any one of items 1 to 3, wherein R^{a}, R^{b} or R^{c} is selected from:

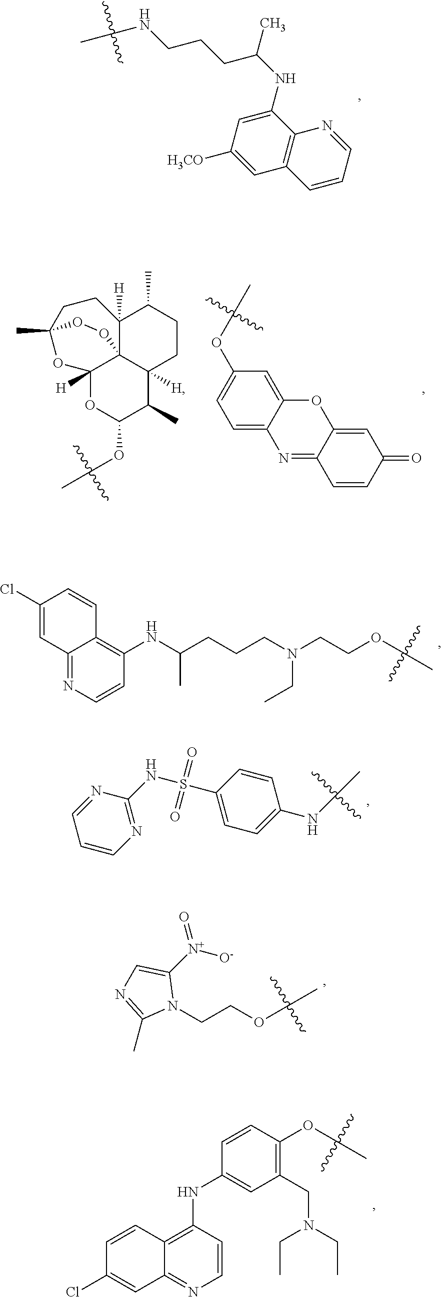

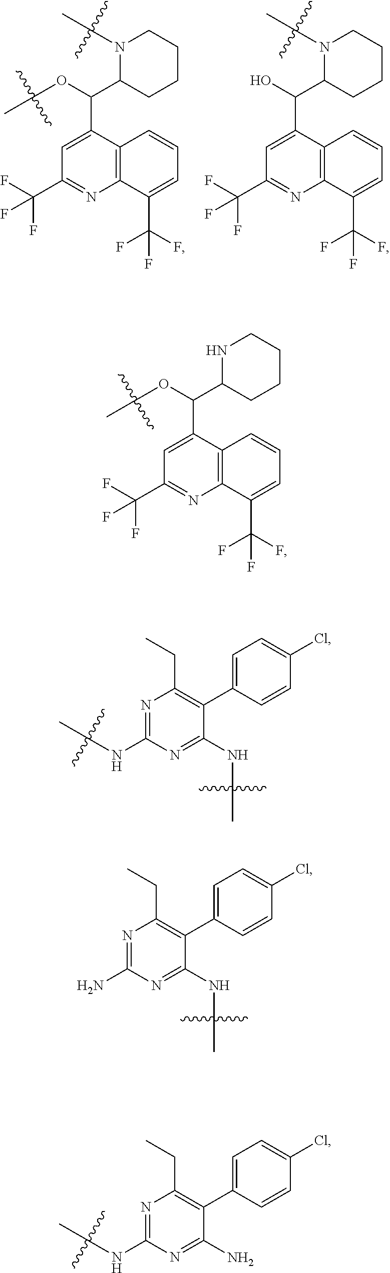

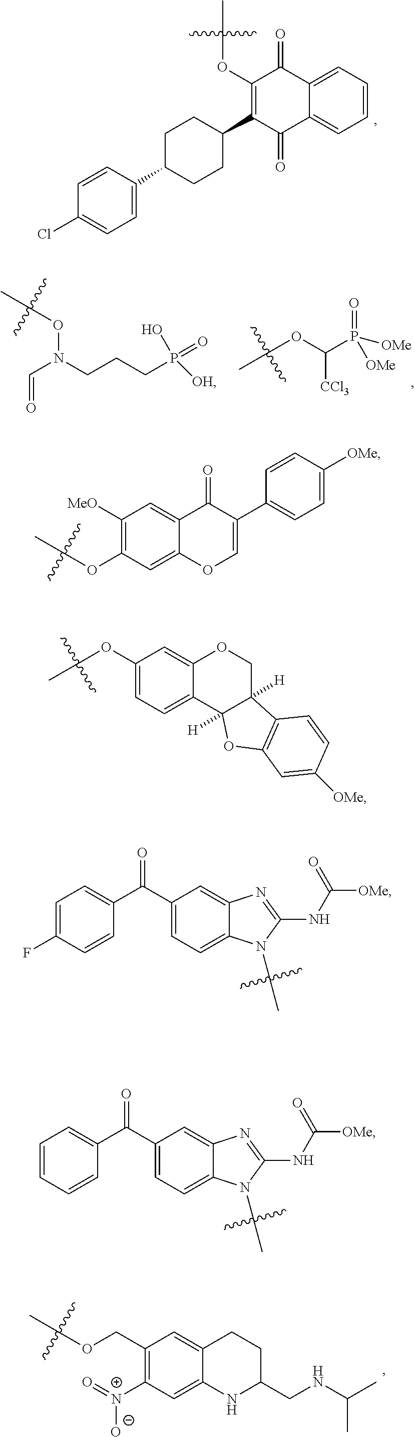

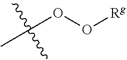

wherein R^{g} is H or substituted or unsubstituted —C₁₋₆ alkyl,

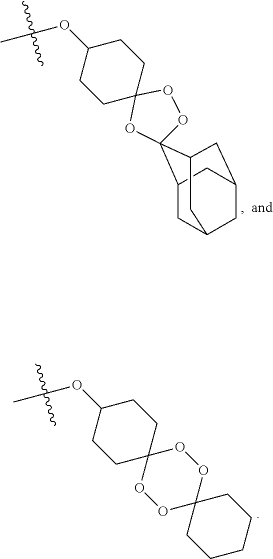 [SMILES: *OC1CCC2(CC1)OOC1(O2)C2CC3CC(C2)CC1C3 ; *OC1CCC2(CC1)OOC1(CCCCC1)OO2]

5. A compound according to any one of items 1 to 4, wherein each of L¹, L² and L³, when present, is substituted or unsubstituted and is independently selected from:

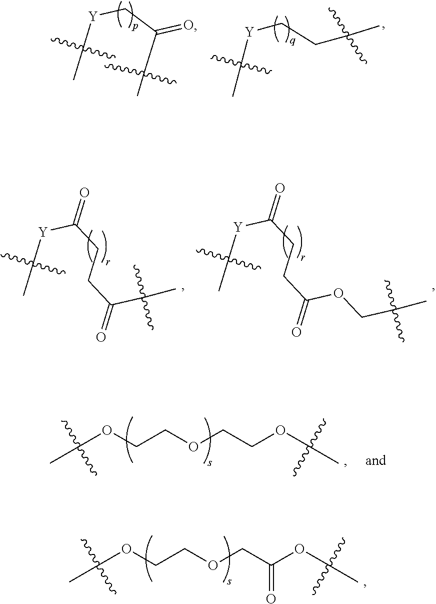

wherein Y is —NR^{f}—, —S—, —O— or —CR^{f1}R^{f2}—, wherein each of R^{f}, R^{f1} and R^{f2} is H or substituted or unsubstituted —C₁₋₆ alkyl; each of p, q, r and s is independently 0, 1, 2, 3 or 4; wherein, when L¹, L² or L³ is substituted, L¹, L² or L³ is substituted with one or more groups selected from Substituent Group A.
6. A compound according to any one of items 1 to 5, wherein each of L¹, L² and L³, when present, is substituted or unsubstituted and is independently selected from:

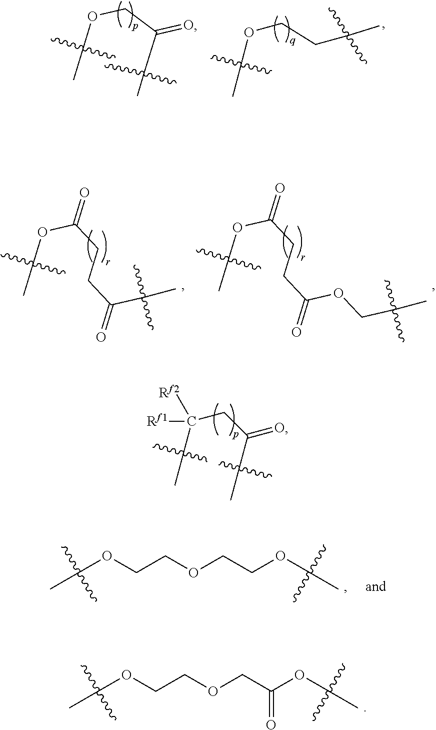

7. A compound according to any one of items 1 to 6, wherein each of L¹, L², and L³, when present, is substituted or unsubstituted and is independently selected from:

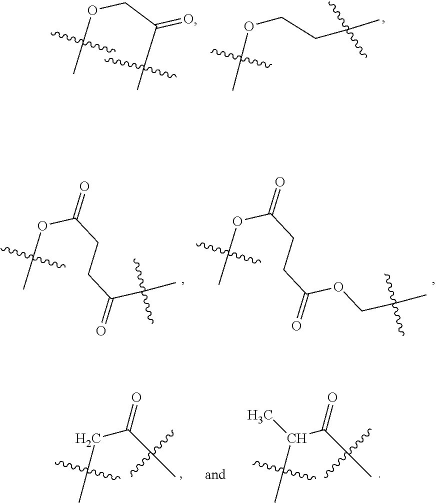

8. A compound according to any one of items 1 to 7, wherein the compound of Formula (I) is a compound of Formula (Ia):

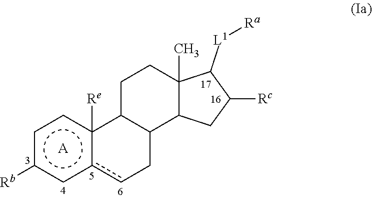

wherein:
R^{a} is selected from:

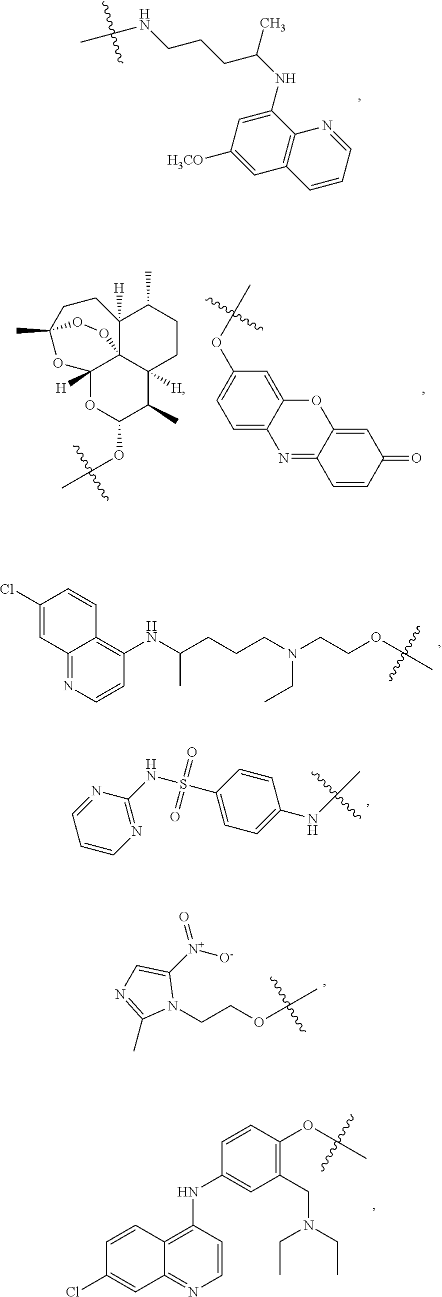

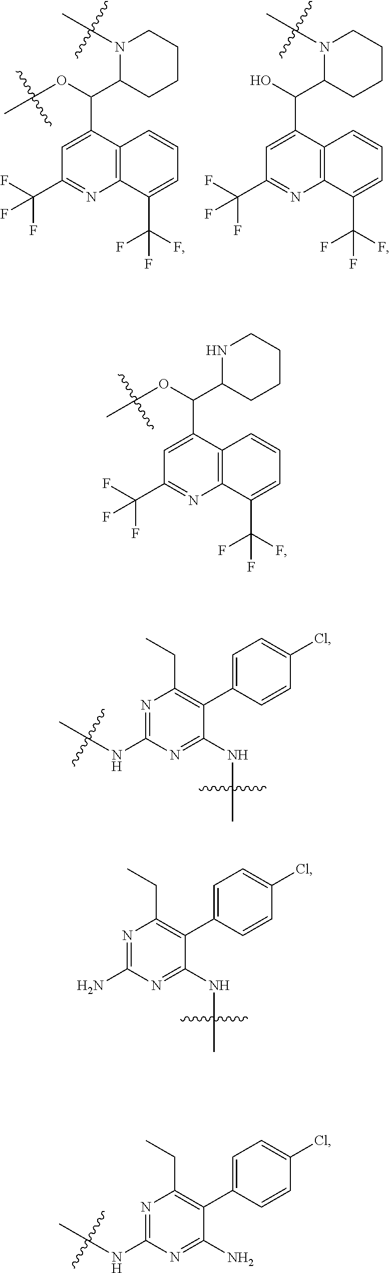

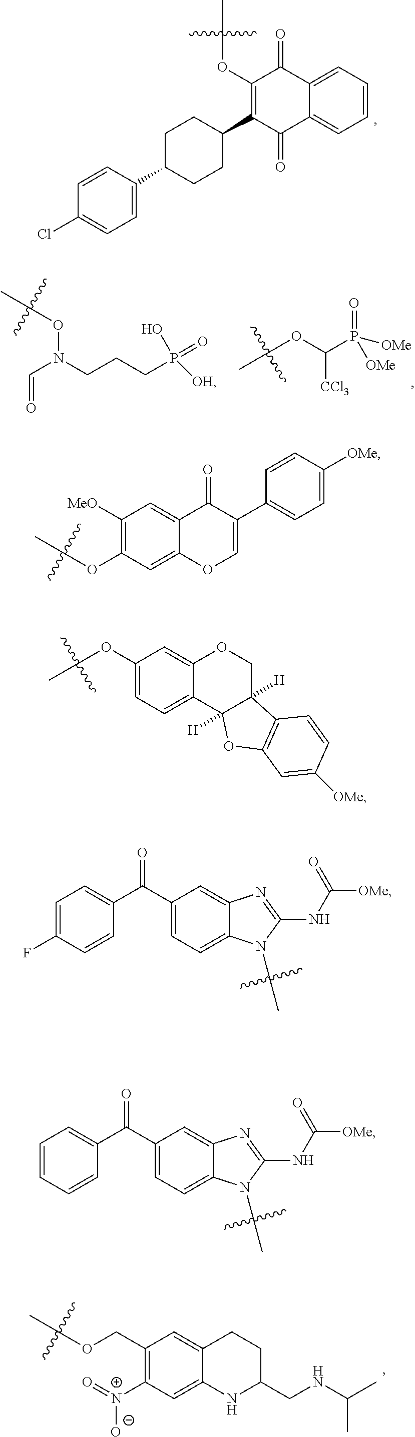

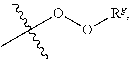

wherein R^{g} is H or substituted or unsubstituted —C₁₋₆ alkyl,

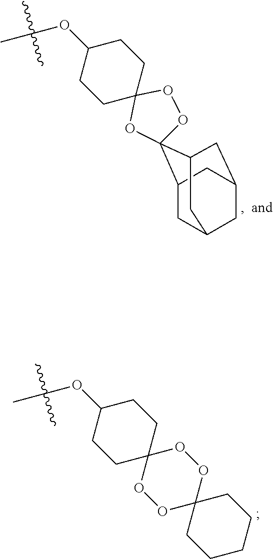 [SMILES: *OC1CCC2(CC1)OOC1(O2)C2CC3CC(C2)CC1C3 ; *OC1CCC2(CC1)OOC1(CCCCC1)OO2]

each of R^{b} and R^{c} is selected from —H, —OH, ═O, substituted or unsubstituted —C₁₋₁₀ alkyl, substituted or unsubstituted —C₂₋₁₀ alkenyl, substituted or unsubstituted —C₂₋₁₀ alkynyl, substituted or unsubstituted —C₃₋₁₀ cycloalkyl; substituted or unsubstituted —C₅₋₁₄ cycloalkenyl, or substituted or unsubstituted —C₈₋₁₄ cycloalkynyl, wherein, when R^{b} or R^{c} is substituted, R^{b} or R^{c} is substituted with one or more groups selected from Substituent Group A;
R^{e} is H or CH₃, or R^{e} is absent;
L¹ may be substituted or unsubstituted and is independently selected from:

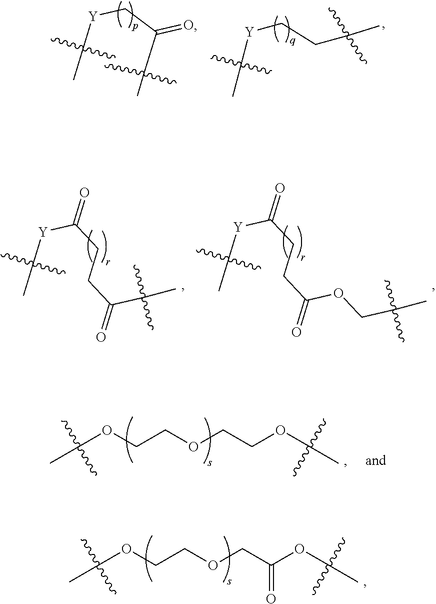

wherein Y is —NR^{f}—, —S—, —O— or —CR^{f1}R^{f2}—, wherein each of R^{f}, R^{f1} and R^{f2} is H or substituted or unsubstituted —C₁₋₆ alkyl; each of p, q, r and s is independently 0, 1, 2, 3 or 4; wherein, when L¹ is substituted, L¹ is substituted with one or more groups selected from Substituent Group A;
ring A may be a saturated or unsaturated ring, or ring A may be an aromatic ring, provided that when ring A is an aromatic ring, the C5-C6 bond is a single bond, R^{e} is absent, and R^{b} is not ═O;
[symbol] is a single bond or a double bond; provided that when the C4-C5 or C5-C10 bond is a double bond, the C5-C6 bond is a single bond; when the C5-C6 bond is a double bond, the C4-C5 and C5-C10 bonds are single bonds; and when the C3 forms a double bond with C2 or C4, R^{b} is not ═O;
or a stereoisomer thereof, or a salt thereof.
9. A compound according to any one of items 1 to 7, wherein the compound of Formula (I) is a compound of Formula (Ib):

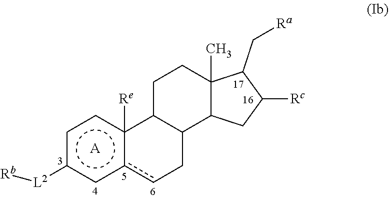

wherein:
each of R^{a} and R^{c} is selected from —H, —OH, ═O, substituted or unsubstituted —C₁₋₁₀ alkyl, substituted or unsubstituted —C₂₋₁₀ alkenyl, substituted or unsubstituted —C₂₋₁₀ alkynyl, substituted or unsubstituted —C₃₋₁₀ cycloalkyl, substituted or unsubstituted —C₅₋₁₄ cycloalkenyl, or substituted or unsubstituted —C₈₋₁₄ cycloalkynyl, wherein, when R^{a} or R^{c} is substituted, R^{a} or R^{c} is substituted with one or more groups selected from Substituent Group A;
   R^{b} is selected from:

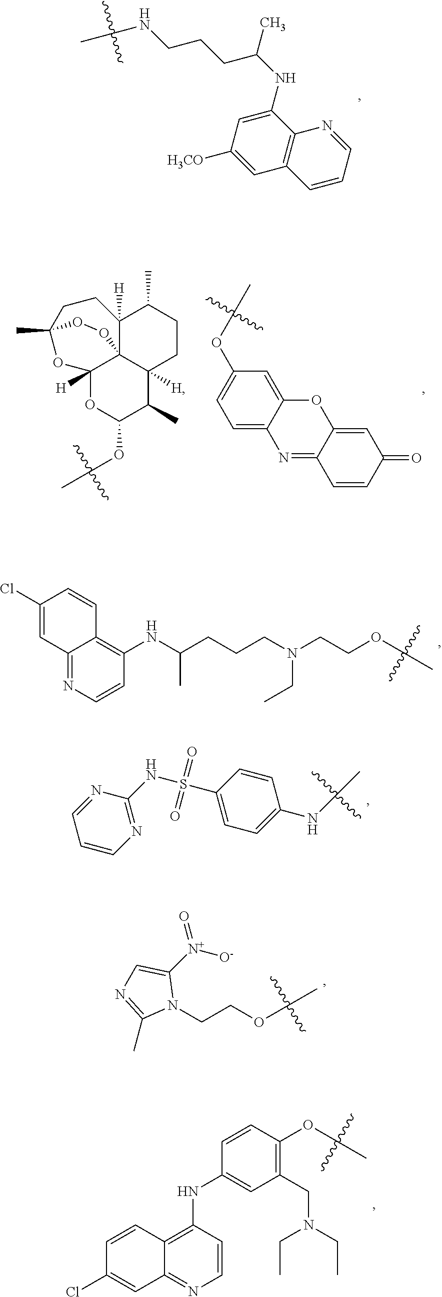

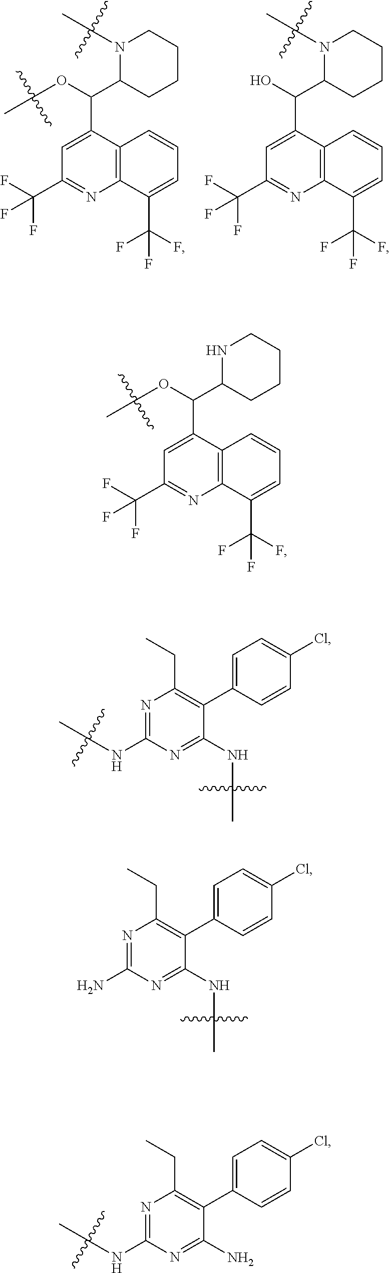

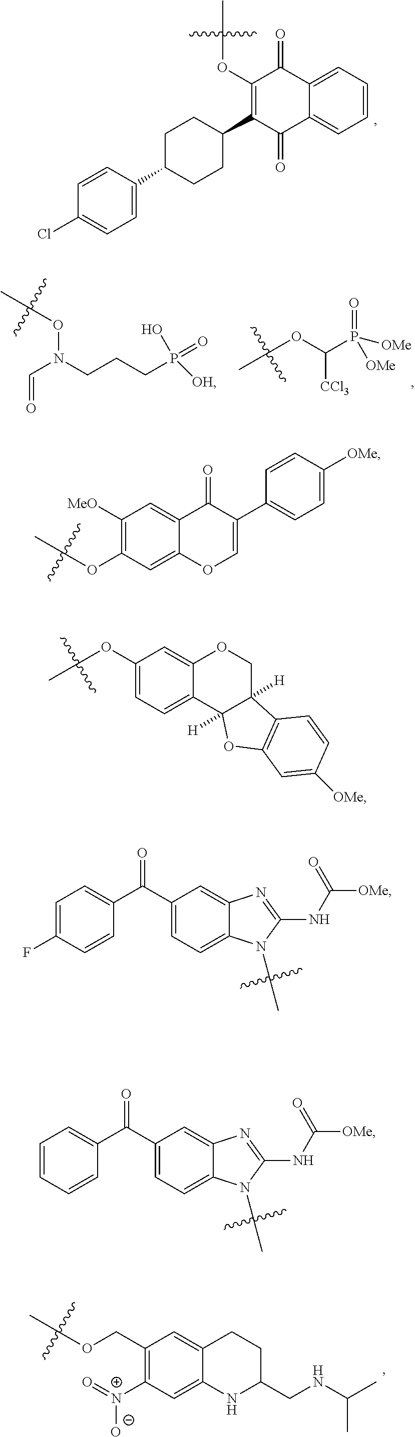

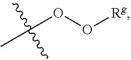

wherein R^{g} is H or substituted or unsubstituted —C₁₋₆ alkyl,

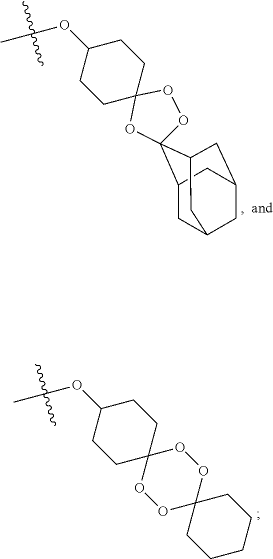 [SMILES: *OC1CCC2(CC1)OOC1(O2)C2CC3CC(C2)CC1C3 ; *OC1CCC2(CC1)OOC1(CCCCC1)OO2]

R^{e} is H or CH₃, or R^{e} is absent;
L² may be substituted or unsubstituted and is independently selected from:

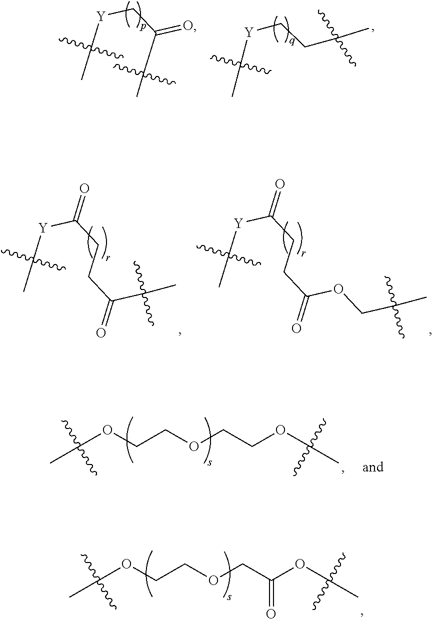

wherein Y is —NR^{f}—, —S—, —O— or —CR^{f1}R^{f2}—, wherein each of R^{f}, R^{f1} and R^{f2} is H or substituted or unsubstituted —C₁₋₆ alkyl; each of p, q, r and s is independently 0, 1, 2, 3 or 4; wherein, when L² is substituted, L² is substituted with one or more groups selected from Substituent Group A;
ring A may be a saturated or unsaturated ring, or ring A may be an aromatic ring, provided that when ring A is an aromatic ring, the C₅-C₆ bond is a single bond, R^{e} is absent, and R^{b} is not ═O;
[symbol] is a single bond or a double bond; provided that when the C4-C5 or C5-C10 bond is a double bond, the C5-C6 bond is a single bond; when the C5-C6 bond is a double bond, the C4-C5 and C5-C10 bonds are single bonds; and when the C3 forms a double bond with C2 or C4, R^{b} is not ═O;
   or a stereoisomer thereof, or a salt thereof.
10. A compound according to any one of items 1 to 7, wherein the compound of Formula (I) is a compound of Formula (Ic):

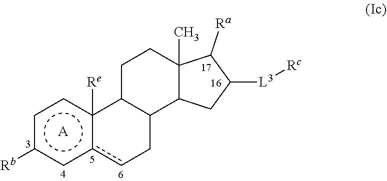

wherein:
each of R^{a} and R^{b} is selected from —H, —OH, ═O, substituted or unsubstituted —C₁₋₁₀ alkyl, substituted or unsubstituted —C₂₋₁₀ alkenyl, substituted or unsubstituted —C₂₋₁₀ alkynyl, substituted or unsubstituted —C₃₋₁₀ cycloalkyl; substituted or unsubstituted —C₅₋₁₄ cycloalkenyl, or substituted or unsubstituted —C₈₋₁₄ cycloalkynyl, wherein, when R^{a} or R^{b} is substituted, R^{a} or R^{b} is substituted with one or more groups selected from Substituent Group A;
   R^{c} is selected from:

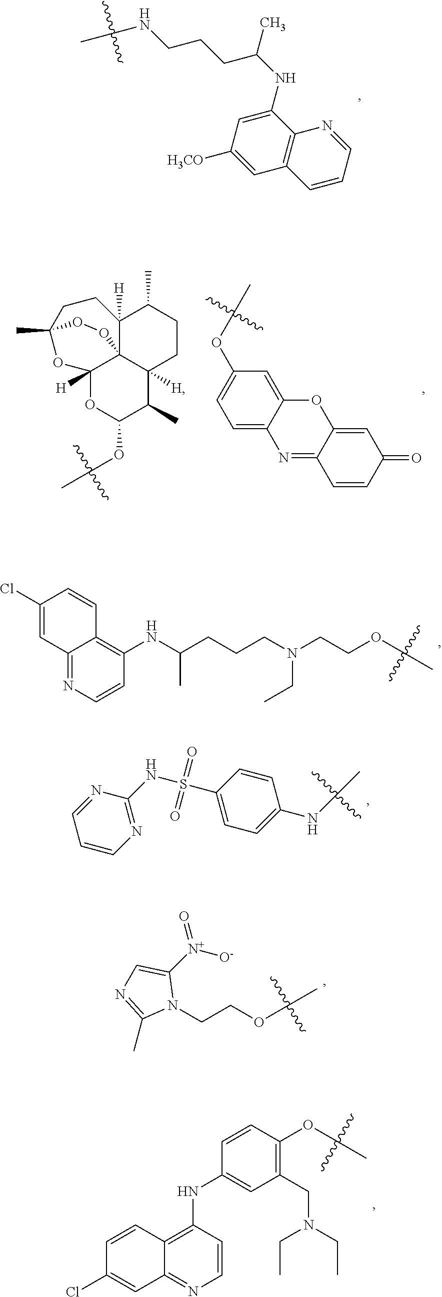

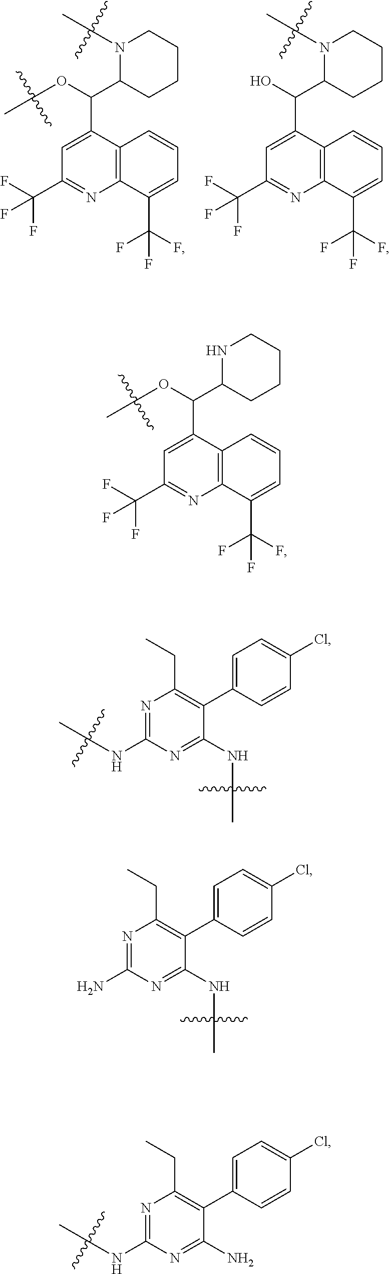

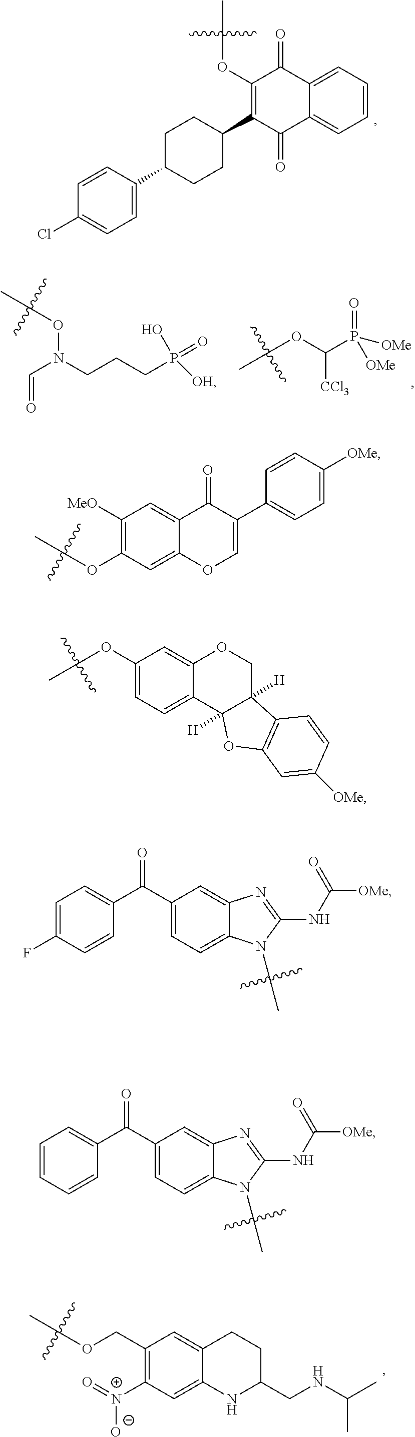

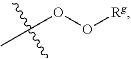

wherein R^{g} is H or substituted or unsubstituted —C₁₋₆ alkyl,

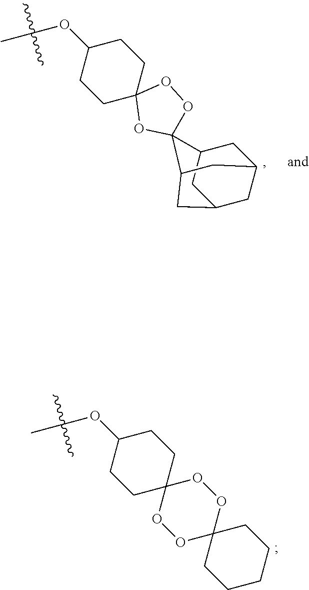 [SMILES: *OC1CCC2(CC1)OOC1(O2)C2CC3CC(C2)CC1C3 ; *OC1CCC2(CC1)OOC1(CCCCC1)OO2]

R^{e} is H or CH₃, or R^{e} is absent;
L³ may be substituted or unsubstituted and is independently selected from:

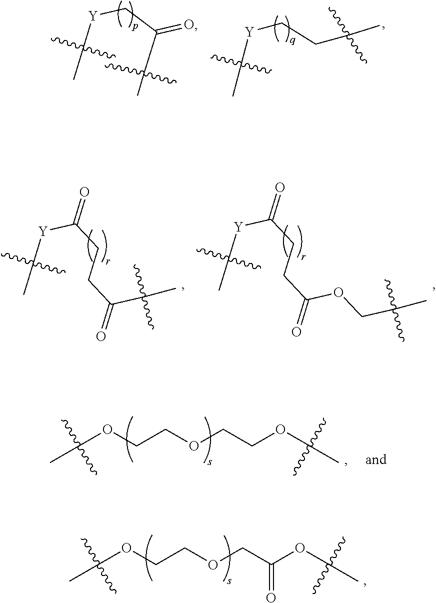

wherein Y is —NR^{f}—, —S—, —O— or —CR^{f1}R^{f2}—, wherein each of R^{f}, R^{f1} and R^{f2} is H or substituted or unsubstituted —C₁₋₆ alkyl; each of p, q, r and s is independently 0, 1, 2, 3 or 4; wherein, when L³ is substituted, L³ is substituted with one or more groups selected from Substituent Group A;
ring A may be a saturated or unsaturated ring, or ring A may be an aromatic ring, provided that when ring A is an aromatic ring, the C5-C6 bond is a single bond, R^{e} is absent, and R^{b} is not ═O;
[symbol] is a single bond or a double bond; provided that when the C4-C5 or C5-C10 bond is a double bond, the C5-C6 bond is a single bond; when the C5-C6 bond is a double bond, the C4-C5 and C5-C10 bonds are single bonds; and when the C3 forms a double bond with C2 or C4, R^{b} is not ═O;
or a stereoisomer thereof, or a salt thereof.
11. A compound according to any one of items 8 to 10, wherein Formula (Ia), Formula (Ib) or Formula (Ic) comprises a steroid group selected from:

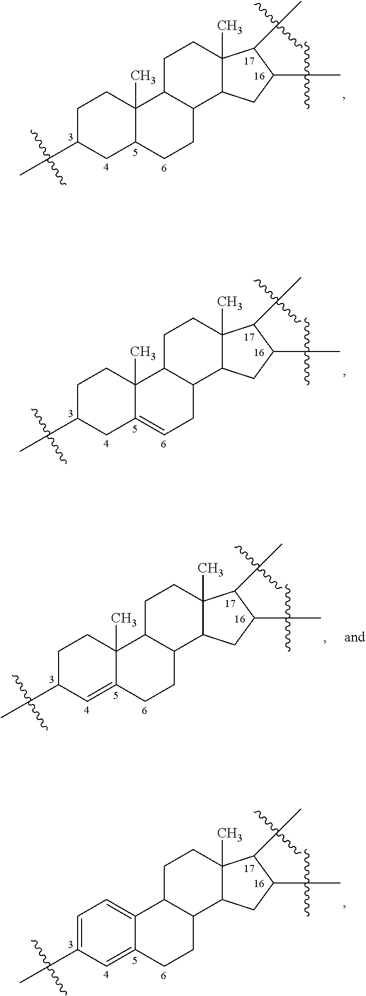 [SMILES: *C1CCC2(C)C(CCC3C2CCC2(C)C(*)C(*)CC32)C1 ; *C1CCC2(C)C(=CCC3C2CCC2(C)C(*)C(*)CC32)C1 ; *C1C=C2CCC3C(CCC4(C)C(*)C(*)CC34)C2(C)CC1 ; *c1ccc2c(c1)CCC1C2CCC2(C)C(*)C(*)CC12]

wherein the groups at C3, C16 and C17 may be attached in the α or β configuration on the respective ring.
12. A compound according to any one of items 8 to 11, wherein each of L¹, L² and L³, when present, is substituted or unsubstituted and is independently selected from:

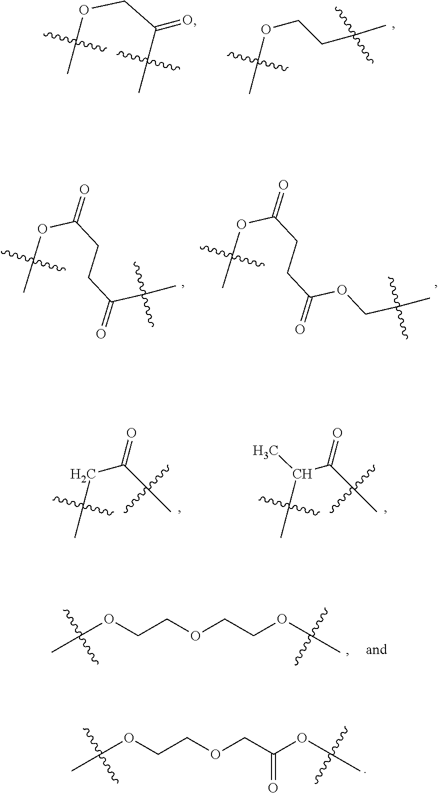

13. A compound according to any one of items 8 to 12, wherein R^{a}, R^{b} or R^{c} is selected from:

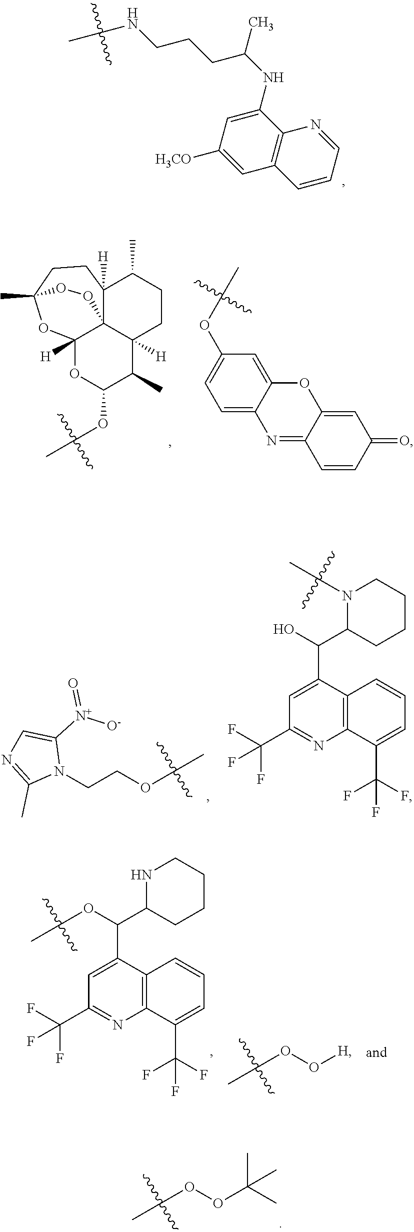

14. A compound according to item 1, wherein the compound is selected from:

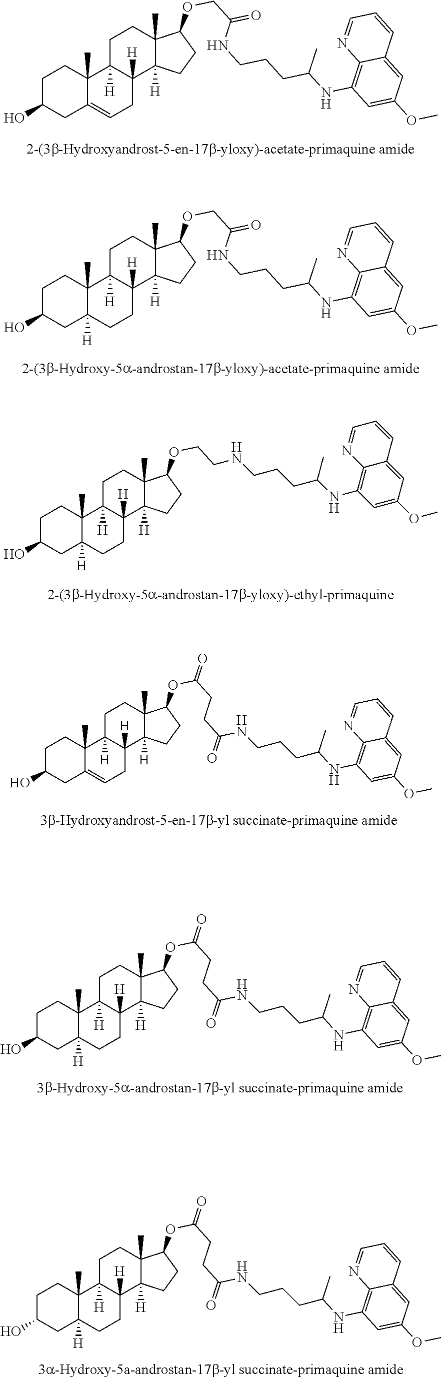

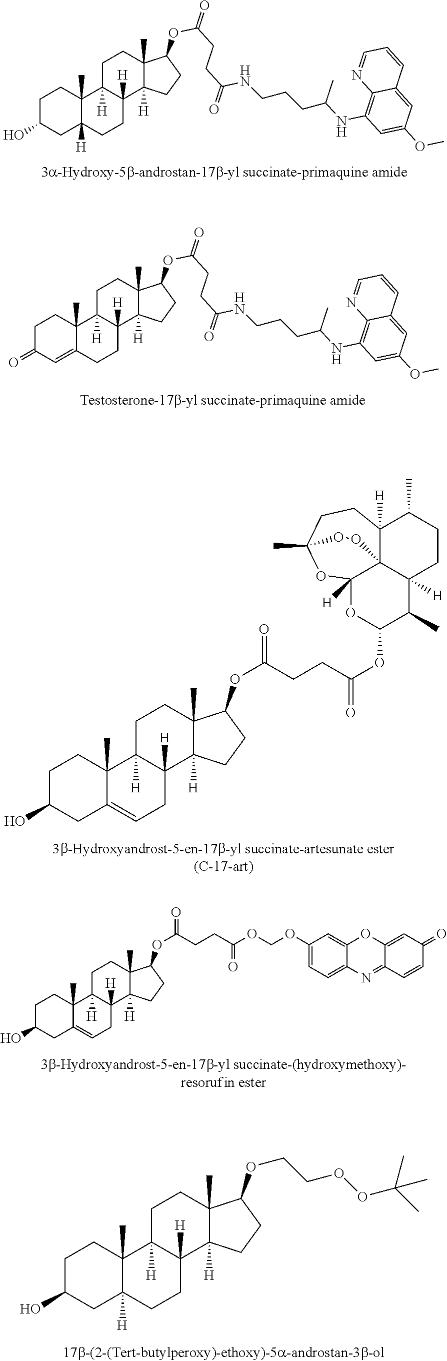

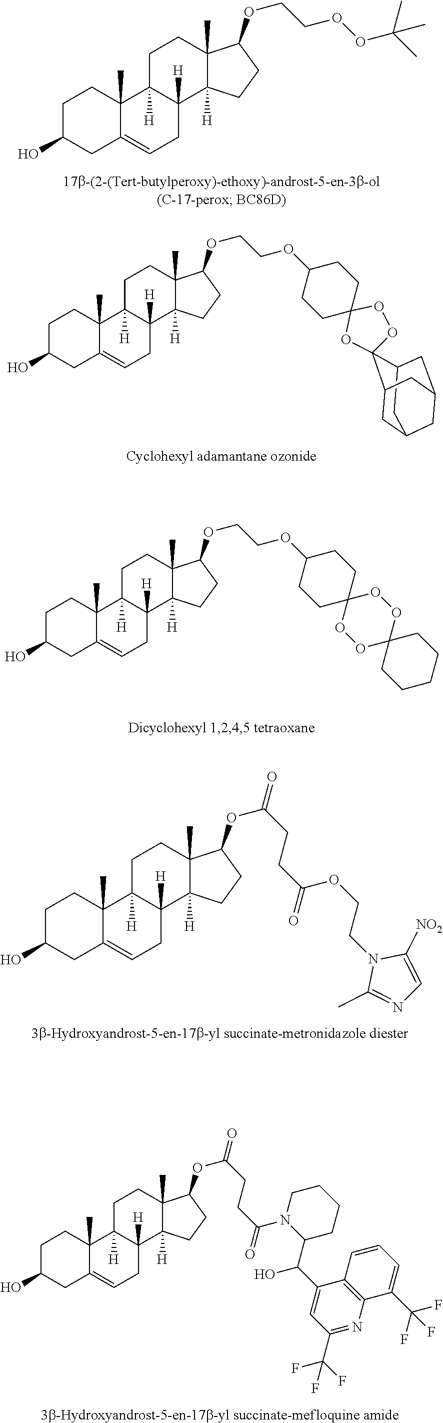

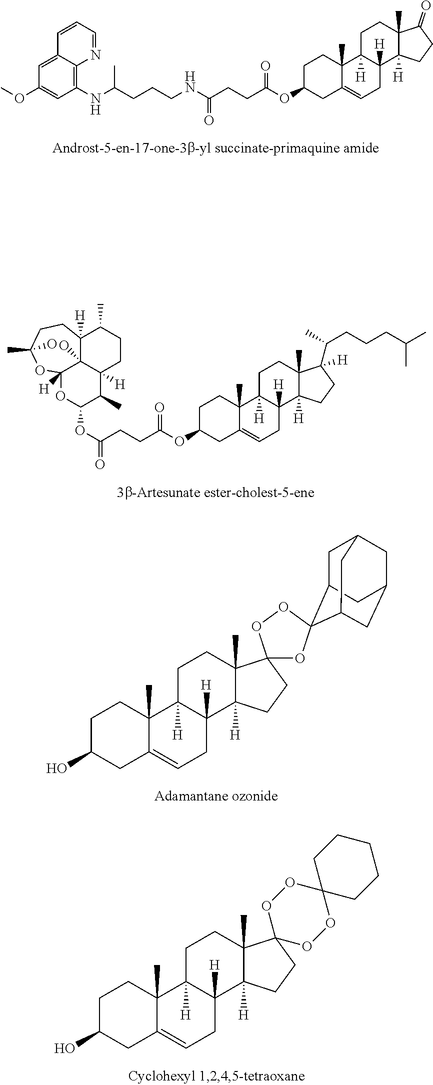

or a salt thereof.
15. A compound of Formula (I) according to any one of items 1 to 14, or a pharmaceutically acceptable salt thereof, for use in treating or preventing an apicomplexan parasite infection in a subject.
16. A composition comprising a compound of Formula (I) according to any one of items 1 to 14, or a salt thereof, and a suitable carrier, adjuvant or diluent.
17. A pharmaceutical composition comprising a compound of Formula (I) according to any one of items 1 to 14, or a pharmaceutically acceptable salt thereof, and a pharmaceutically acceptable carrier, adjuvant or diluent.
18. A method of treating or preventing a parasite infection in a subject, the method comprising administering to the subject an effective amount of a compound of Formula (I) according to any one of items 1 to 14, or a pharmaceutically acceptable salt thereof.
19. A method of inhibiting the proliferation of a parasite, the method comprising contacting the apicomplexan parasite with an effective amount of a compound of Formula (I) according to any one of items 1 to 14 or a salt thereof.
20. Use of a compound of Formula (I) according to any one of items 1 to 14, or a pharmaceutically acceptable salt thereof, in the manufacture of a medicament for the treatment or prevention of a parasite infection in a subject.
21. The compound for use of item 15, the method of item 18 or 19, or the use of item 20, wherein the parasite infection is an Apicomplexan parasite infection.
22. The compound for use, method or use of item 21, wherein the Apicomplexan parasite infection is caused by an organism selected from Plasmodium spp., Toxoplasma spp., Eimeria spp., Isospora spp., Theileria spp., Babesia spp., Sarcocystis spp., and Cryptosporidium spp.
23. The compound for use, method or use of item 22, wherein the apicomplexan parasite infection is caused by Plasmodium falciparum, Plasmodium vivax, Plasmodium ovale curtisi, Plasmodium ovale wallikeri, Plasmodium malariae, Plasmodium knowlesi or coccidia.
24. The compound for use, method or use of item 22, wherein the apicomplexan parasite infection is caused by Eimeria spp. or Isospora spp.
25. The compound for use, method or use of item 22, wherein the apicomplexan parasite infection is caused by Toxoplasma gondii.

1-46. (canceled)

47. A method of treating or preventing a parasite infection in a subject, the method comprising administering to the subject an effective amount of a compound of Formula (I): 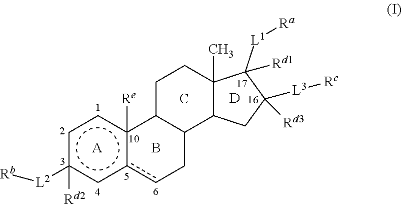
wherein:
each of R^{a} and R^{c} is independently selected from —H, —OH, ═O, substituted or unsubstituted —C₁₋₁₀ alkyl, substituted or unsubstituted —C₂₋₁₀ alkenyl, substituted or unsubstituted —C₂₋₁₀ alkynyl, substituted or unsubstituted —C₃₋₁₀ cycloalkyl, substituted or unsubstituted —C₅₋₁₄ cycloalkenyl, substituted or unsubstituted —C₈₋₁₄ cycloalkynyl, and an anti-parasite moiety; wherein at least one of R^{a} and R^{c} is an anti-parasite moiety;
R^{b} is selected from —H, —OH, ═O, substituted or unsubstituted —C₁₋₁₀ alkyl, substituted or unsubstituted —C₂₋₁₀ alkenyl, substituted or unsubstituted —C₂₋₁₀ alkynyl, substituted or unsubstituted —C₃₋₁₀ cycloalkyl, substituted or unsubstituted —C₅₋₁₄ cycloalkenyl, and substituted or unsubstituted —C₈₋₁₄ cycloalkynyl;
provided that: when R^{a} is —OH, L¹ is absent; when R^{a} is ═O, L¹ and R^{d1} are absent; when R^{b} is —OH, L² is absent; when R^{b} is ═O, L² is absent, R^{d2} is absent, and the C2-C3 bond and the C3-C4 bond are single bonds; when R^{c} is —OH, L³ is absent; when R^{c} is ═O, L³ and R^{d3} are absent;
when R^{a}, R^{b} or R^{c} is substituted, R^{a}, R^{b} or R^{c} is substituted with one or more groups selected from Substituent Group A;
R^{d1}, when present, is H or is a group that, together with R^{a}, forms a substituted or unsubstituted ozonide ring or a substituted or unsubstituted tetraoxane ring, wherein the optional substituent may be a —C₃₋₁₀ cycloalkyl; provided that when R^{d1} and R^{a} form a substituted or unsubstituted ozonide ring or a substituted or unsubstituted tetraoxane ring, L¹ is absent; wherein, when the ozonide or tetraoxane ring is substituted, the ozonide or tetraoxane ring is substituted with one or more —C₃₋₁₀ cycloalkyl groups;
R^{d2}, when present, is H; provided that when R^{d2} is present, the C2-C3 bond and the C3-C4 bond are single bonds;
R^{d3}, when present, is H or is a group that, together with R^{c}, forms a substituted or unsubstituted ozonide ring or a substituted or unsubstituted tetraoxane ring, wherein the optional substituent may be a —C₃₋₁₀ cycloalkyl; provided that when R^{d3} and R^{c} form a substituted or unsubstituted ozonide ring or a substituted or unsubstituted tetraoxane ring, L³ is absent; wherein, when the ozonide or tetraoxane ring is substituted, the ozonide or tetraoxane ring is substituted with one or more —C₃₋₁₀ cycloalkyl groups;
R^{e} is H or CH₃, or R^{e} is absent;
when present, L¹ is a group that provides a covalent linkage between R^{a} and the C17 of ring D;
when present, L² is a group that provides a covalent linkage between R^{b} and the C3 of ring A;
when present, L³ is a group that provides a covalent linkage between R^{c} and the C16 of ring D;
ring A may be a saturated or unsaturated ring, or ring A may be an aromatic ring, provided that when ring A is an aromatic ring, the C5-C6 bond is a single bond, R^{d2} is absent, R^{e} is absent, and R^{b} is not ═O; [symbol]
is a single bond or a double bond; provided that when the C4-C5 or C5-C10 bond is a double bond, the C5-C6 bond is a single bond; when the C5-C6 bond is a double bond, the C4-C5 and C5-C10 bonds are single bonds; and when the C3 forms a double bond with C2 or C4, R^{b} is not ═O;
or a stereoisomer thereof, or a pharmaceutically acceptable salt thereof.

48. A method according to claim 47, wherein Formula (I) is selected from:

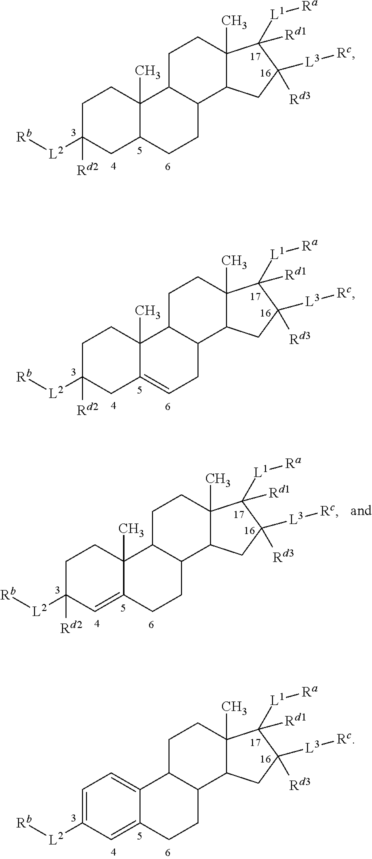

49. A method according to claim 47, wherein Formula (I) is selected from:

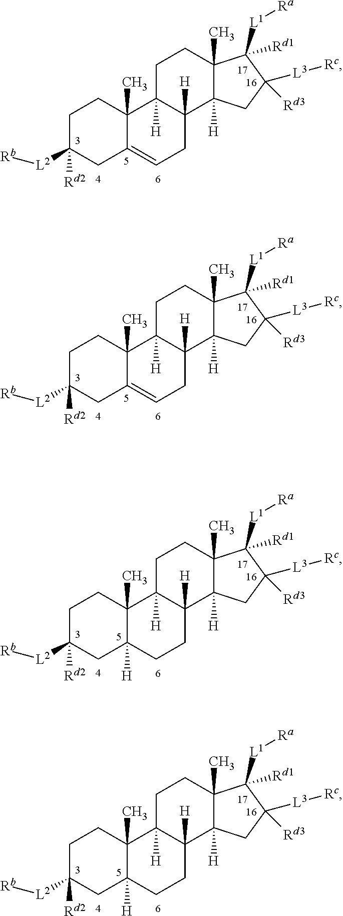

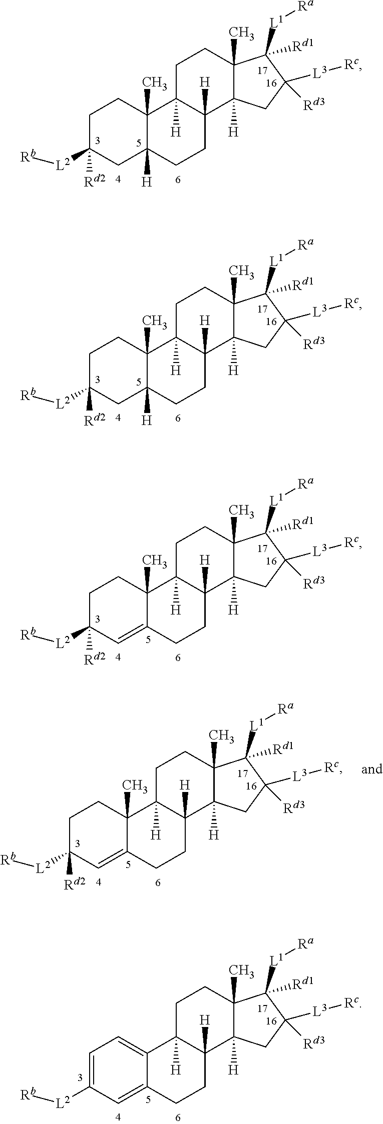

50. A method according to claim 47, wherein the anti-parasite moiety has anti-apicomplexan activity or antihelminth activity.

51. A method according to claim 47, wherein the anti-parasite moiety is an anti-apicomplexan moiety.

52. A method according to claim 47, wherein the anti-parasite moiety R^{a} or R^{c} is selected from: 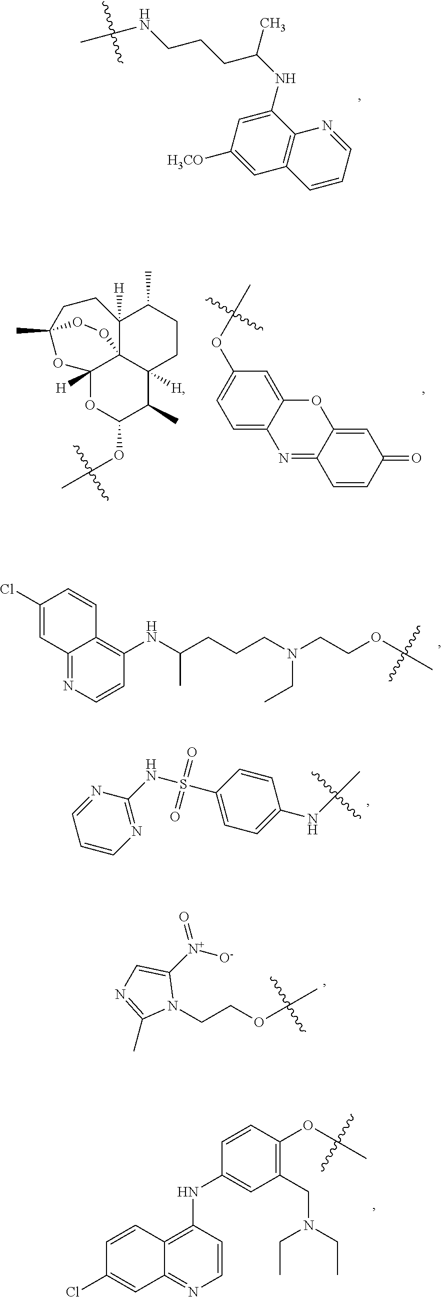 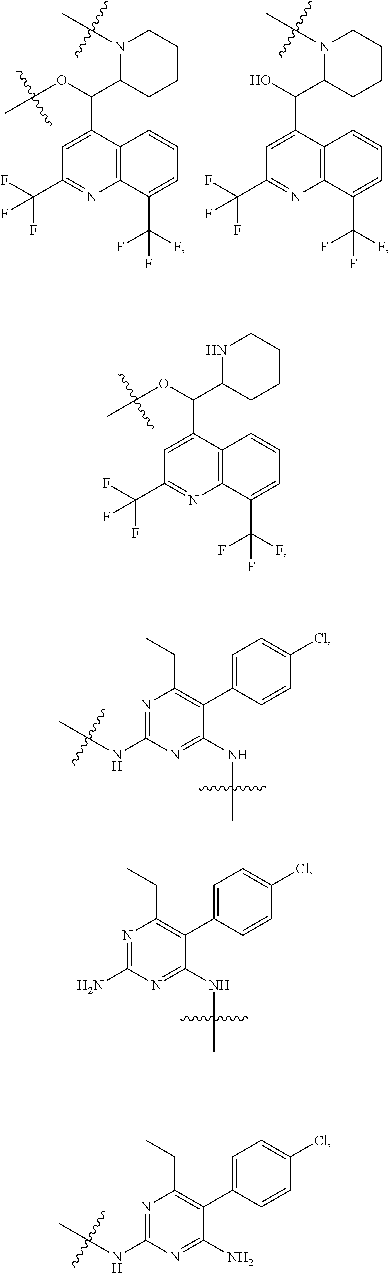 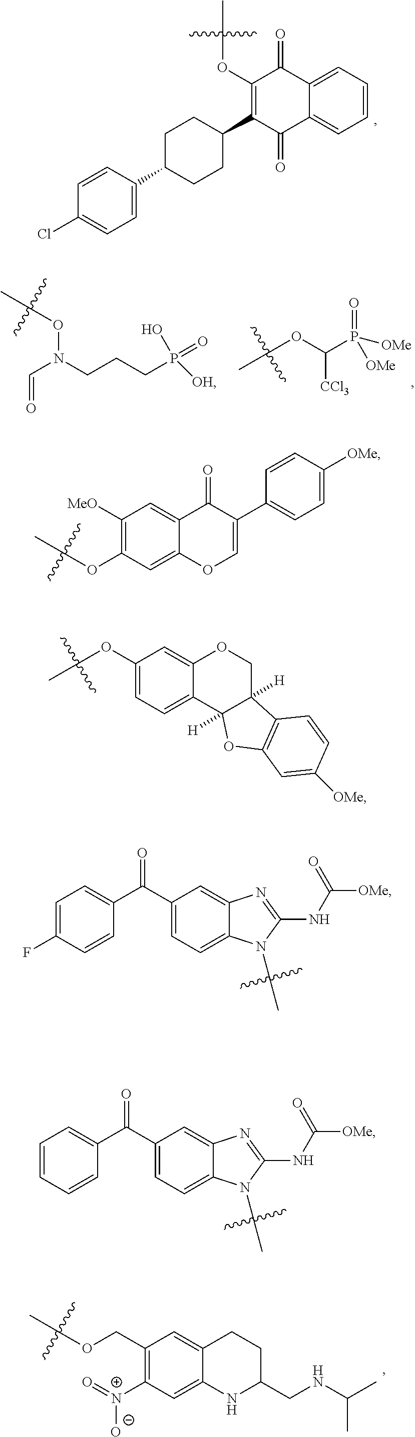 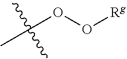
wherein R^{g} is H or substituted or unsubstituted —C₁₋₆ alkyl,

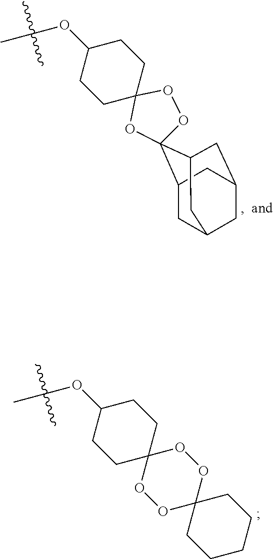 [SMILES: *OC1CCC2(CC1)OOC1(O2)C2CC3CC(C2)CC1C3 ; *OC1CCC2(CC1)OOC1(CCCCC1)OO2]

53. A method according to claim 47, wherein each of L¹, L² and L³, when present, is substituted or unsubstituted and is independently selected from: 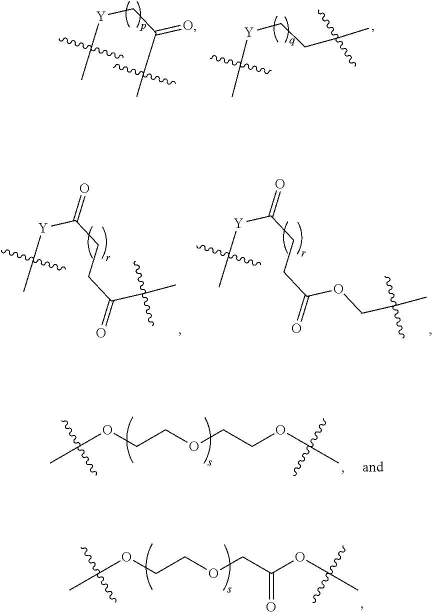
wherein Y is —NR^{f}—, —S—, —O— or —CR^{f1}R^{f2}—, wherein each of R^{f}, R^{f1} and R^{f2} is H or substituted or unsubstituted —C₁₋₆ alkyl; each of p, q, r and s is independently 0, 1, 2, 3 or 4; wherein, when L¹, L² or L³ is substituted, L¹, L² or L³ is substituted with one or more groups selected from Substituent Group A.

54. A method according to claim 47, wherein each of L¹, L² and L³, when present, is substituted or unsubstituted and is independently selected from:

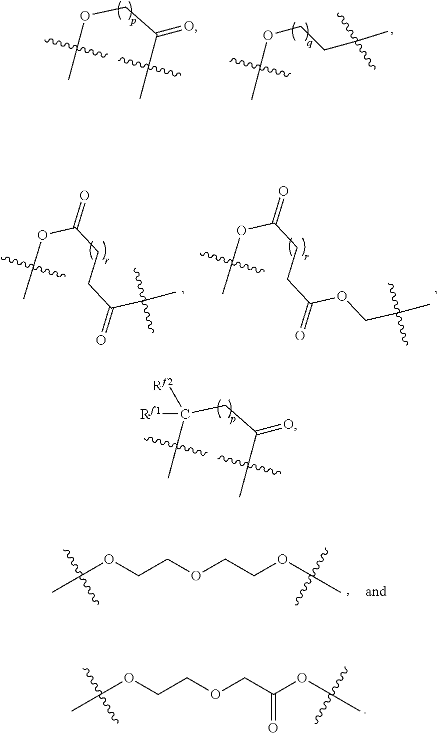

55. A method according to claim 47, wherein each of L¹, L² and L³, when present, is substituted or unsubstituted and is independently selected from:

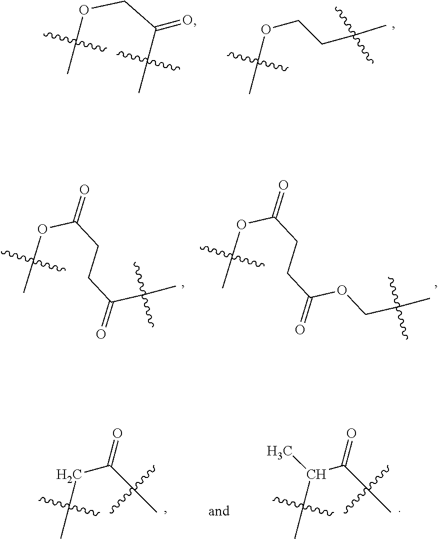

56. A method according to claim 47, wherein the compound of Formula (I) is a compound of Formula (Ia) or (Ic), or a stereoisomer thereof, or a pharmaceutically acceptable salt thereof: 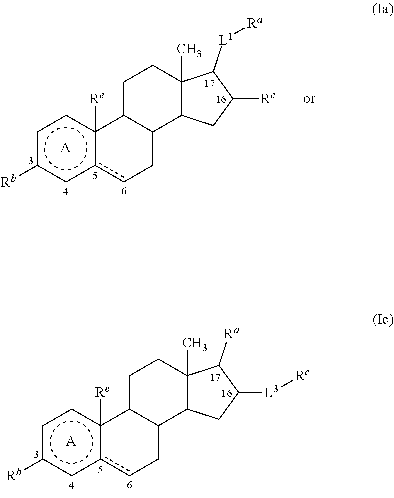
wherein when the compound of Formula (I) is Formula (Ia):
R^{a} is selected from: 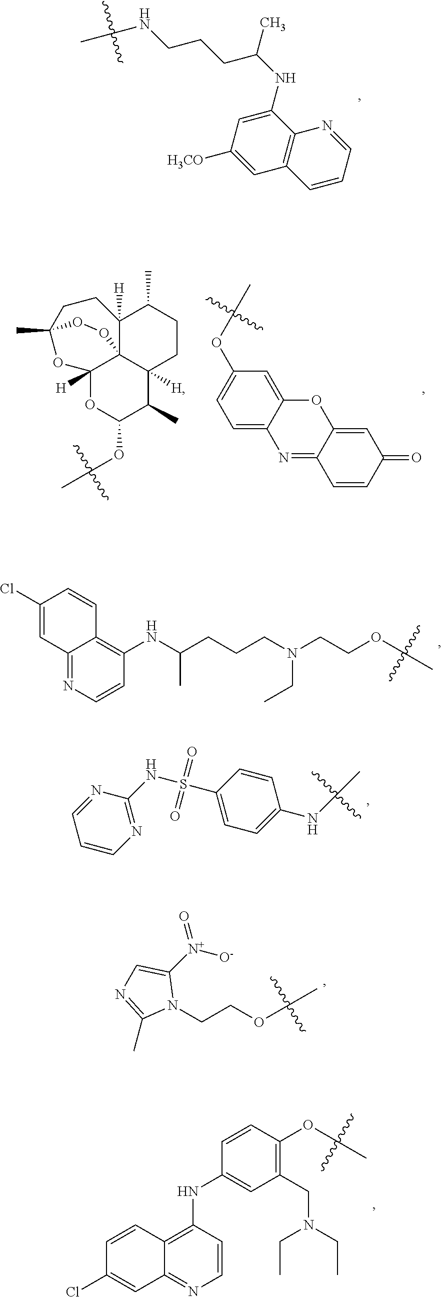 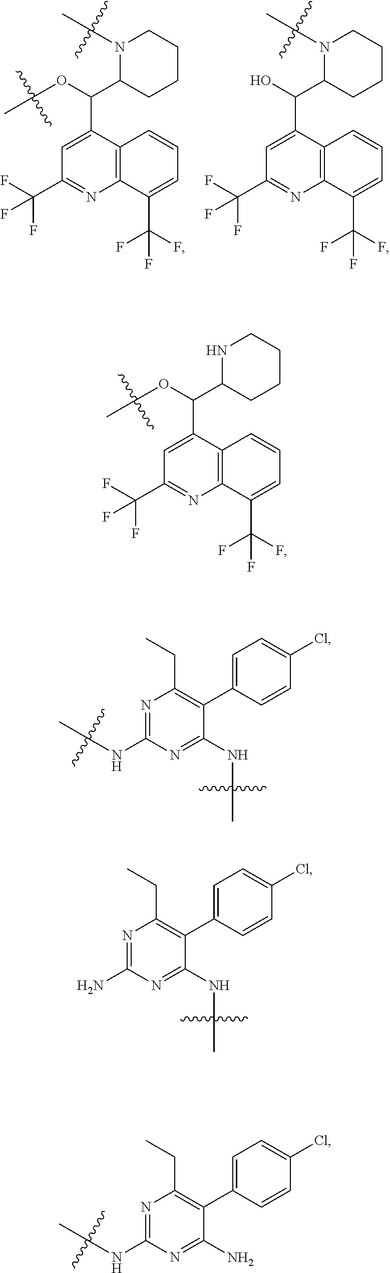 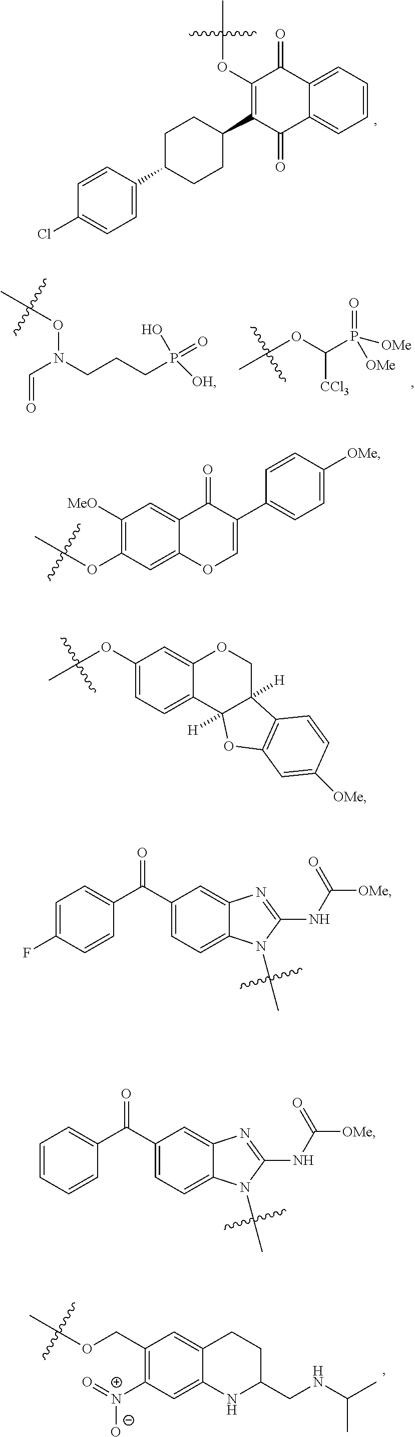 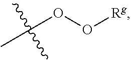
wherein R^{g} is H or substituted or unsubstituted —C₁₋₆ alkyl, 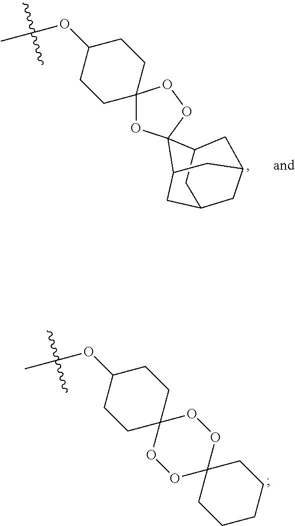 [SMILES: *OC1CCC2(CC1)OOC1(O2)C2CC3CC(C2)CC1C3 ; *OC1CCC2(CC1)OOC1(CCCCC1)OO2]
each of R^{b} and R^{c} is selected from —H, —OH, ═O, substituted or unsubstituted —C₁₋₁₀ alkyl, substituted or unsubstituted —C₂₋₁₀ alkenyl, substituted or unsubstituted —C₂₋₁₀ alkynyl, substituted or unsubstituted —C₃₋₁₀ cycloalkyl; substituted or unsubstituted —C₅₋₁₄ cycloalkenyl, or substituted or unsubstituted —C₈₋₁₄ cycloalkynyl, wherein, when R^{b} or R^{c} is substituted, R^{b} or R^{c} is substituted with one or more groups selected from Substituent Group A;
R^{e} is H or CH₃, or R^{e} is absent;
L¹ may be substituted or unsubstituted and is independently selected from: 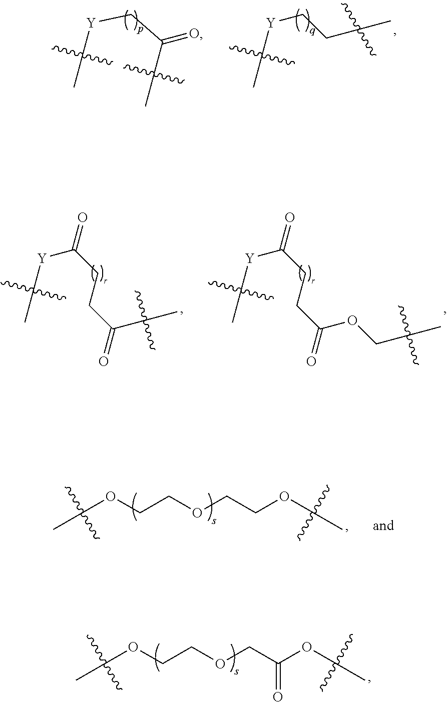
wherein Y is —NR^{f}—, —S—, —O— or —CR^{f1}R^{f2}—, wherein each of R^{f}, R^{f1} and R^{f2} is H or substituted or unsubstituted —C₁₋₆ alkyl; each of p, q, r and s is independently 0, 1, 2, 3 or 4; wherein, when L¹ is substituted, L¹ is substituted with one or more groups selected from Substituent Group A;
ring A may be a saturated or unsaturated ring, or ring A may be an aromatic ring, provided that when ring A is an aromatic ring, the C5-C6 bond is a single bond, R^{e} is absent, and R^{b} is not ═O; [symbol]
is a single bond or a double bond; provided that when the C4-C5 or C5-C10 bond is a double bond, the C5-C6 bond is a single bond; when the C5-C6 bond is a double bond, the C4-C5 and C5-C10 bonds are single bonds; and when the C3 forms a double bond with C2 or C4, R^{b} is not ═O;
and wherein when the compound of Formula (I) is Formula (Ic):
each of R^{a} and R^{b} is selected from —H, —OH, ═O, substituted or unsubstituted —C₁₋₁₀ alkyl, substituted or unsubstituted —C₂₋₁₀ alkenyl, substituted or unsubstituted —C₂₋₁₀ alkynyl, substituted or unsubstituted —C₃₋₁₀ cycloalkyl; substituted or unsubstituted —C₅₋₁₄ cycloalkenyl, or substituted or unsubstituted —C₈₋₁₄ cycloalkynyl, wherein, when R^{a} or R^{b} is substituted, R^{a} or R^{b} is substituted with one or more groups selected from Substituent Group A;
R^{c} is selected from: 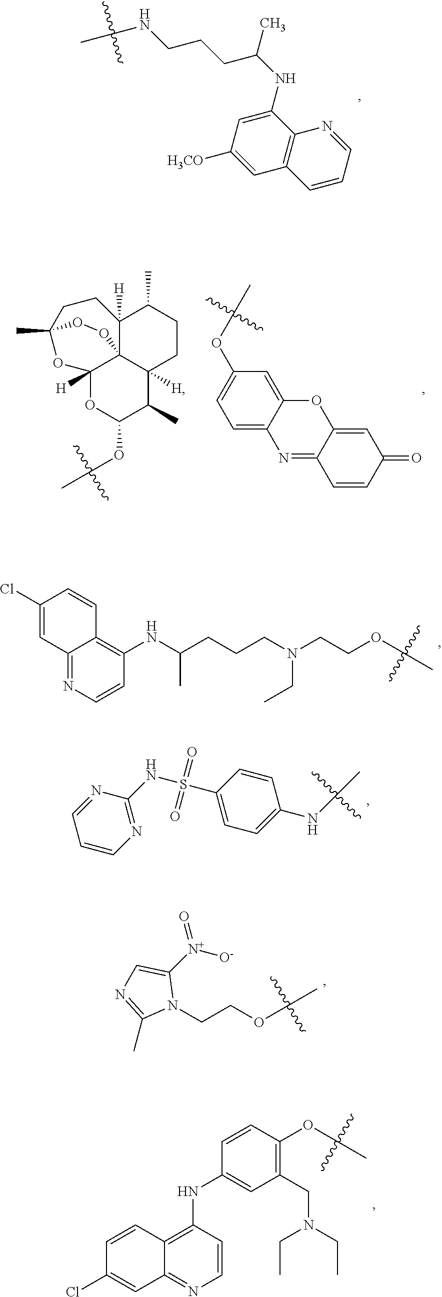 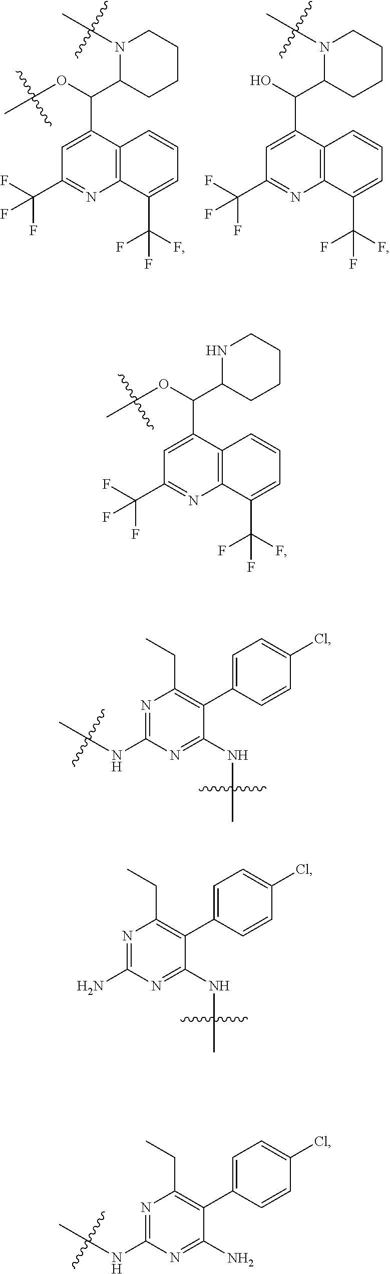 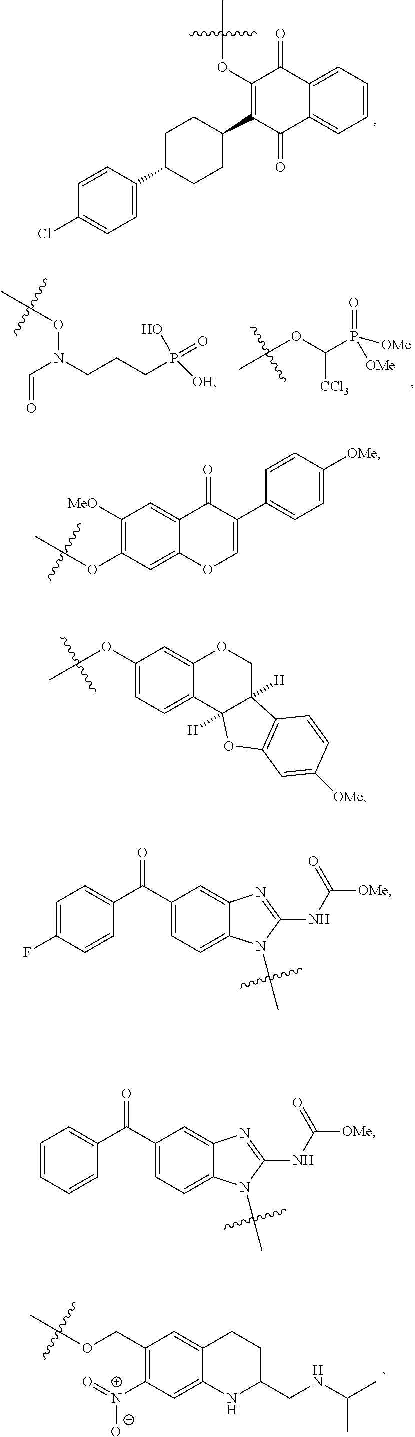 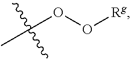
wherein R^{g} is H or substituted or unsubstituted —C₁₋₆ alkyl, 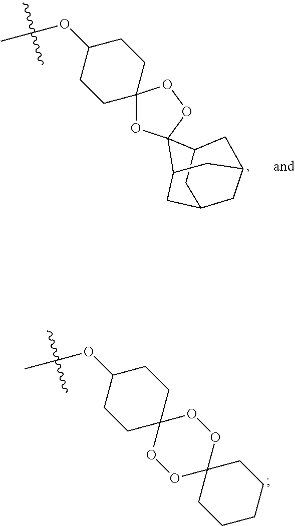 [SMILES: *OC1CCC2(CC1)OOC1(O2)C2CC3CC(C2)CC1C3 ; *OC1CCC2(CC1)OOC1(CCCCC1)OO2]
R^{e} is H or CH₃, or R^{e} is absent;
L³ may be substituted or unsubstituted and is independently selected from: 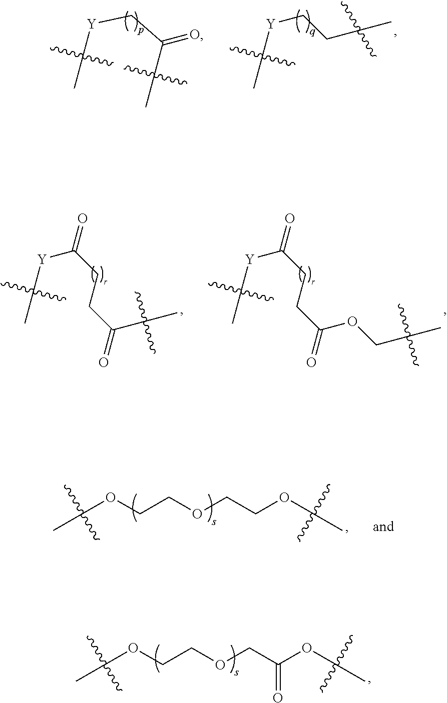
wherein Y is —NR^{f}—, —S—, —O— or —CR^{f1}R^{f2}—, wherein each of R^{f}, R^{f1} and R^{f2} is H or substituted or unsubstituted —C₁₋₆ alkyl; each of p, q, r and s is independently 0, 1, 2, 3 or 4; wherein, when L³ is substituted, L³ is substituted with one or more groups selected from Substituent Group A;
ring A may be a saturated or unsaturated ring, or ring A may be an aromatic ring, provided that when ring A is an aromatic ring, the C5-C6 bond is a single bond, R^{e} is absent, and R^{b} is not ═O; [symbol]
is a single bond or a double bond; provided that when the C4-C5 or C5-C10 bond is a double bond, the C5-C6 bond is a single bond; when the C5-C6 bond is a double bond, the C4-C5 and C5-C10 bonds are single bonds; and when the C3 forms a double bond with C2 or C4, R^{b} is not ═O.

57. A method according to claim 56, wherein Formula (Ia) or Formula (Ic) comprises a steroid group selected from: 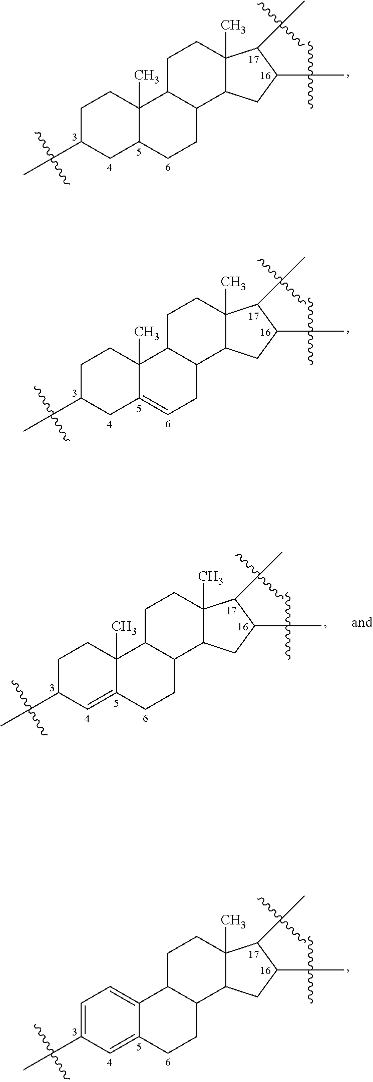 [SMILES: *C1CCC2(C)C(CCC3C2CCC2(C)C(*)C(*)CC32)C1 ; *C1CCC2(C)C(=CCC3C2CCC2(C)C(*)C(*)CC32)C1 ; *C1C=C2CCC3C(CCC4(C)C(*)C(*)CC34)C2(C)CC1 ; *c1ccc2c(c1)CCC1C2CCC2(C)C(*)C(*)CC12]
wherein the groups at C3, C16 and C17 may be attached in the α or β configuration on the respective ring.

58. A method according to claim 56, wherein each of L¹ and L³, when present, is substituted or unsubstituted and is independently selected from:

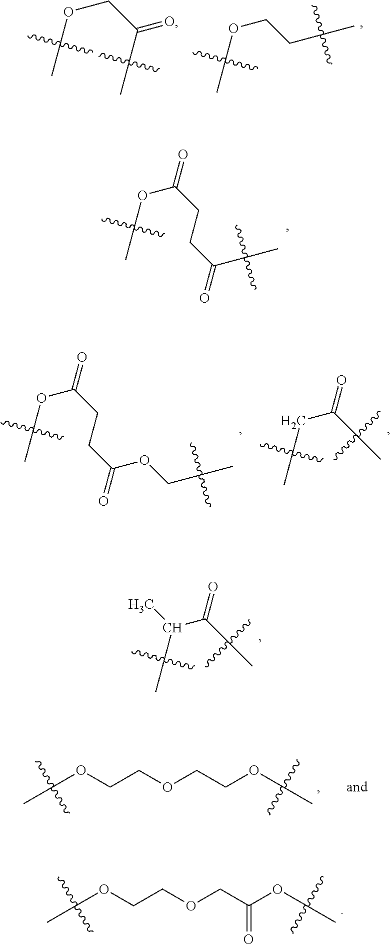

59. A method according to claim 56, wherein R^{a} or R^{c} is selected from:

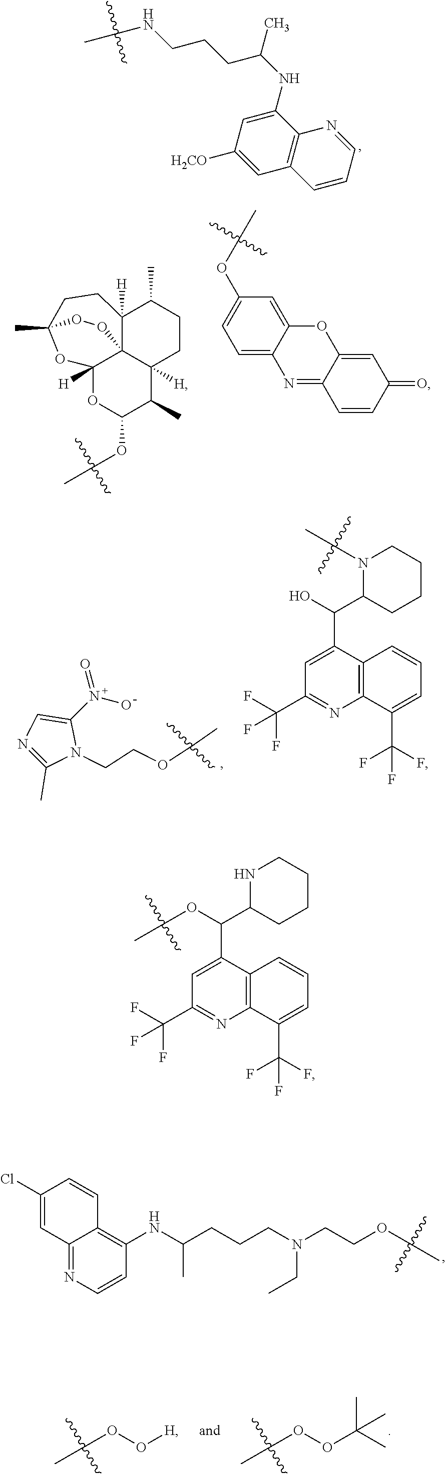

60. A method according to claim 47, wherein the compound is selected from: 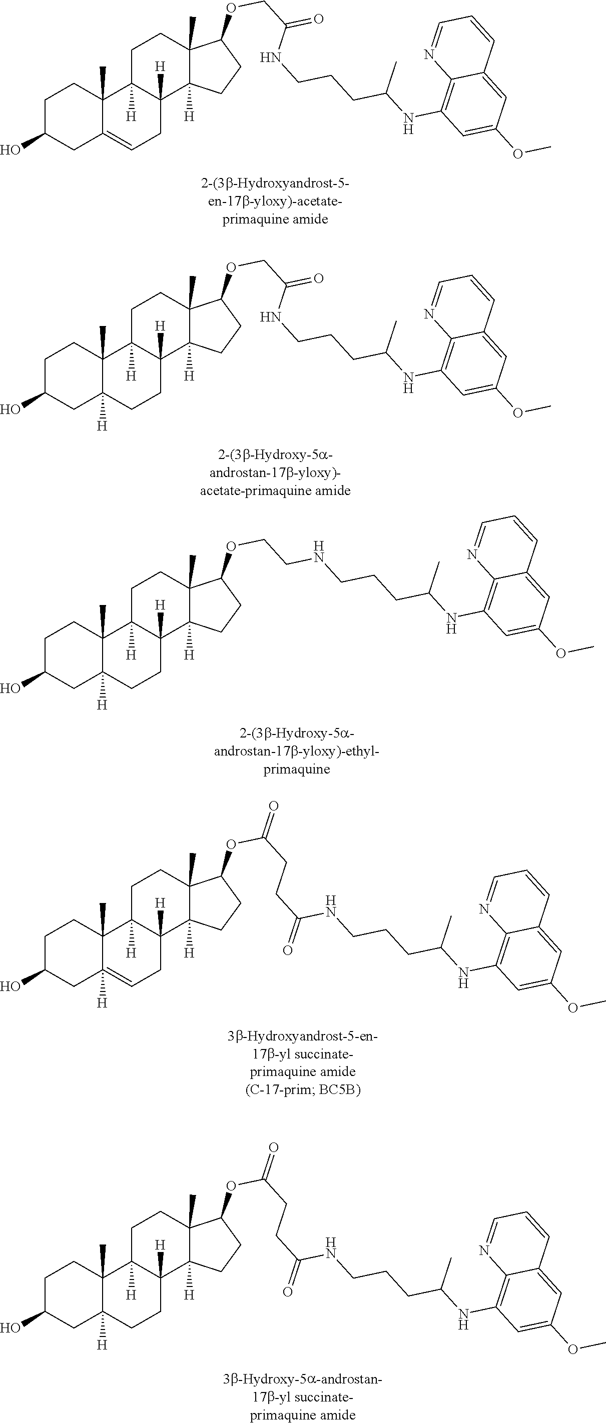 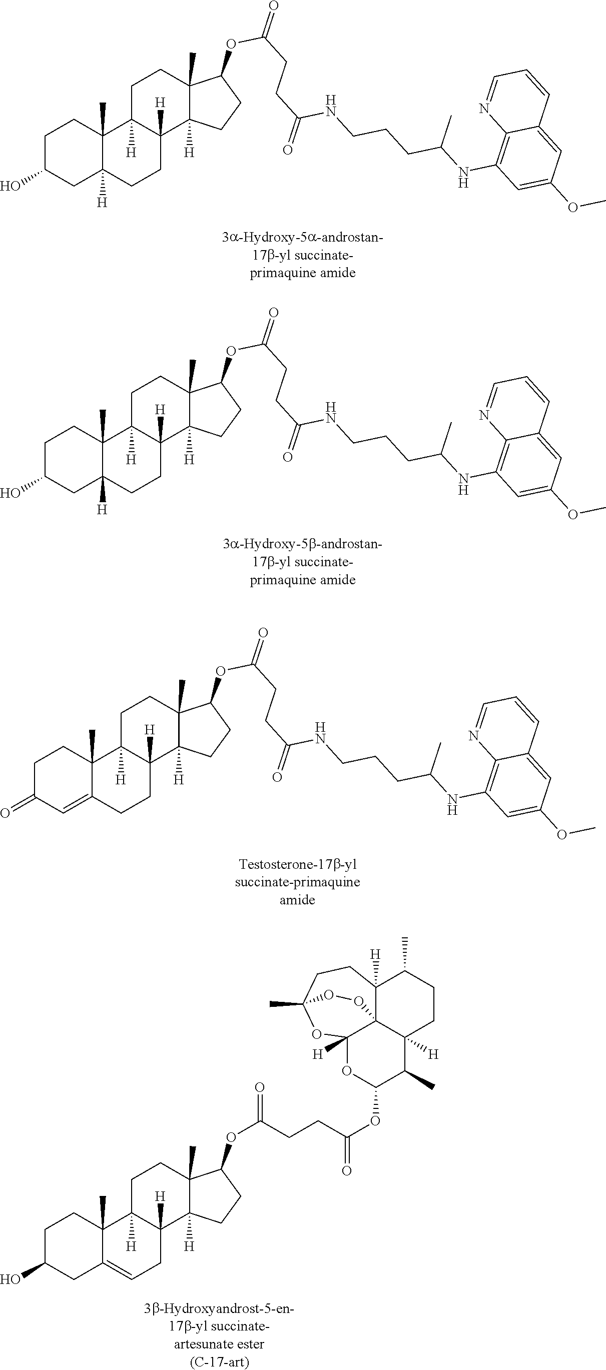 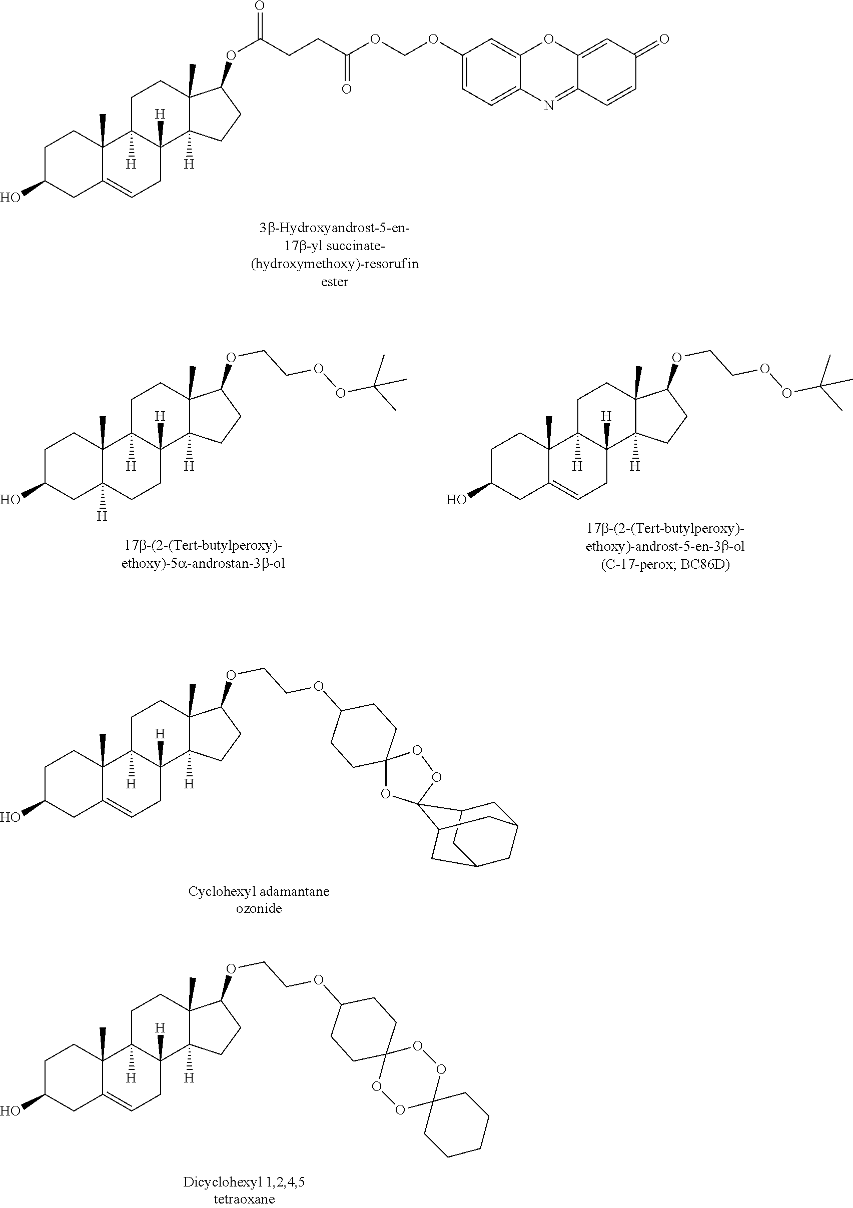 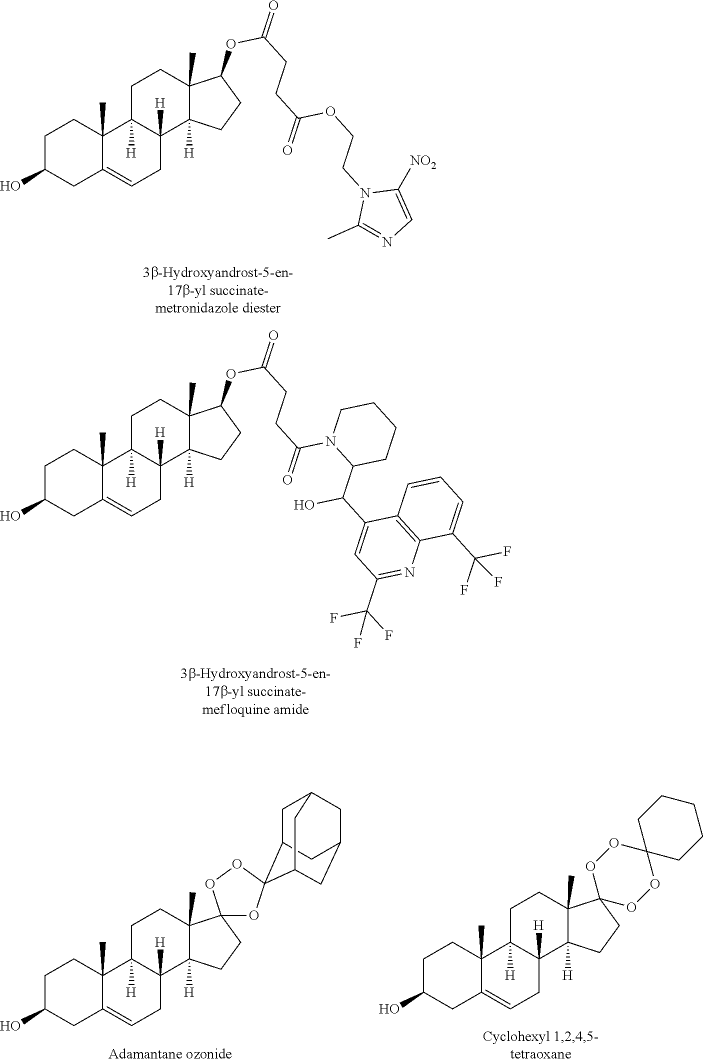
or a pharmaceutically acceptable salt thereof.

61. A method according to claim 47, wherein the parasite infection is a protozoan parasite infection or a helminth infection.

62. A method according to claim 61, wherein the protozoan parasite infection is selected from a flagellate parasite infection, a ciliate parasite infection, an amoeba parasite infection, or an apicomplexan parasite infection.

63. A method according to claim 62, where the protozoan parasite infection is caused by an organism selected from Giardia spp., Trichomonas spp., Leishmania spp., Trypanosoma spp., Balantidium coli, Entamoeba histolytica, Plasmodium spp., Toxoplasma spp., Eimeria spp., Isospora spp., Theileria spp., Babesia spp., Sarcocystis spp., and Cryptosporidium spp.

64. A method according to claim 62, wherein the apicomplexan parasite infection is caused by an organism selected from Plasmodium spp., Toxoplasma spp., Eimeria spp., Isospora spp., Theileria spp., Babesia spp., Sarcocystis spp., and Cryptosporidium spp.

65. A method according to claim 61, wherein the protozoan parasite infection is caused by a kinetoplastid parasite.

66. A method according to claim 64, wherein:
the apicomplexan parasite infection is caused by Plasmodium falciparum, Plasmodium vivax, Plasmodium ovale curtisi, Plasmodium ovale wallikeri, Plasmodium malariae, Plasmodiumr knowlesi or coccidia; or
the apicomplexan parasite infection is caused by Eimeria spp., Isospora spp.; Theileria spp., Babesia spp., Sarcocystis spp., or Cryptosporidium spp; or
the apicomplexan parasite infection is caused by Toxoplasma gondii.

67. A compound of Formula (I) or a pharmaceutically acceptable salt thereof, for use in treating or preventing a parasite infection in a subject; wherein Formula (I) is: 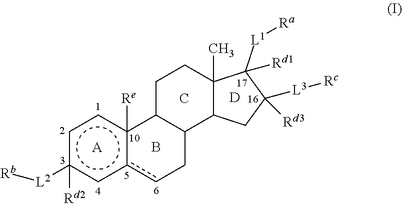
wherein:
each of R^{a} and R^{c} is independently selected from —H, —OH, ═O, substituted or unsubstituted —C₁₋₁₀ alkyl, substituted or unsubstituted —C₂₋₁₀ alkenyl, substituted or unsubstituted —C₂₋₁₀ alkynyl, substituted or unsubstituted —C₃₋₁₀ cycloalkyl, substituted or unsubstituted —C₅₋₁₄ cycloalkenyl, substituted or unsubstituted —C₈₋₁₄ cycloalkynyl, and an anti-parasite moiety; wherein at least one of R^{a} and R^{c} is an anti-parasite moiety;
R^{b} is selected from —H, —OH, ═O, substituted or unsubstituted —C₁₋₁₀ alkyl, substituted or unsubstituted —C₂₋₁₀ alkenyl, substituted or unsubstituted —C₂₋₁₀ alkynyl, substituted or unsubstituted —C₃₋₁₀ cycloalkyl, substituted or unsubstituted —C₅₋₁₄ cycloalkenyl, and substituted or unsubstituted —C₈₋₁₄ cycloalkynyl;
provided that: when R^{a} is —OH, L¹ is absent; when R^{a} is ═O, L¹ and R^{d1} are absent; when R^{b} is —OH, L² is absent; when R^{b} is ═O, L² is absent, R^{d2} is absent, and the C2-C3 bond and the C3-C4 bond are single bonds; when R^{c} is —OH, L³ is absent; when R^{c} is ═O, L³ and R^{d3} are absent;
when R^{a}, R^{b} or R^{c} is substituted, R^{a}, R^{b} or R^{c} is substituted with one or more groups selected from Substituent Group A;
R^{d1}, when present, is H or is a group that, together with R^{a}, forms a substituted or unsubstituted ozonide ring or a substituted or unsubstituted tetraoxane ring, wherein the optional substituent may be a —C₃₋₁₀ cycloalkyl; provided that when R^{d1} and R^{a} form a substituted or unsubstituted ozonide ring or a substituted or unsubstituted tetraoxane ring, L¹ is absent; wherein, when the ozonide or tetraoxane ring is substituted, the ozonide or tetraoxane ring is substituted with one or more —C₃₋₁₀ cycloalkyl groups;
R^{d2}, when present, is H; provided that when R^{d2} is present, the C2-C3 bond and the C3-C4 bond are single bonds:
R^{d3}, when present, is H or is a group that, together with R^{e}, forms a substituted or unsubstituted ozonide ring or a substituted or unsubstituted tetraoxane ring, wherein the optional substituent may be a —C₃₋₁₀ cycloalkyl; provided that when R^{d3} and R^{c} form a substituted or unsubstituted ozonide ring or a substituted or unsubstituted tetraoxane ring, L³ is absent; wherein, when the ozonide or tetraoxane ring is substituted, the ozonide or tetraoxane ring is substituted with one or more —C₃₋₁₀ cycloalkyl groups;
R^{e} is H or CH₃, or R^{e} is absent;
when present, L¹ is a group that provides a covalent linkage between R^{a} and the C17 of ring D;
when present, L² is a group that provides a covalent linkage between R^{b} and the C3 of ring A;
when present, L³ is a group that provides a covalent linkage between R^{c} and the C16 of ring D;
ring A may be a saturated or unsaturated ring, or ring A may be an aromatic ring, provided that when ring A is an aromatic ring, the C5-C6 bond is a single bond, R^{d2} is absent, R^{e} is absent, and R^{b} is not ═O; [symbol]
is a single bond or a double bond; provided that when the C4-C5 or C5-C10 bond is a double bond, the C5-C6 bond is a single bond; when the C5-C6 bond is a double bond, the C4-C5 and C5-C10 bonds are single bonds; and when the C3 forms a double bond with C2 or C4, R^{b} is not ═O;
or a stereoisomer thereof, or a pharmaceutically acceptable salt thereof.

68. A method of inhibiting the proliferation of a parasite, the method comprising contacting the parasite with an effective amount of a compound of Formula (I): 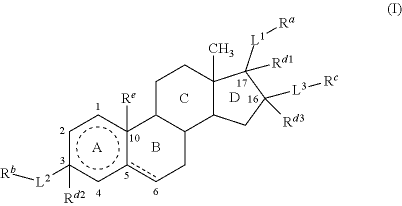
wherein:
each of R^{a} and R^{c} is independently selected from —H, —OH, ═O, substituted or unsubstituted —C₁₋₁₀ alkyl, substituted or unsubstituted —C₂₋₁₀ alkenyl, substituted or unsubstituted —C₂₋₁₀ alkynyl, substituted or unsubstituted —C₃₋₁₀ cycloalkyl, substituted or unsubstituted —C₅₋₁₄ cycloalkenyl, substituted or unsubstituted —C₈₋₁₄ cycloalkynyl, and an anti-parasite moiety; wherein at least one of R^{a} and R^{c} is an anti-parasite moiety;
R^{b} is selected from —H, —OH, ═O, substituted or unsubstituted —C₁₋₁₀ alkyl, substituted or unsubstituted —C₂₋₁₀ alkenyl, substituted or unsubstituted —C₂₋₁₀ alkynyl, substituted or unsubstituted —C₃₋₁₀ cycloalkyl, substituted or unsubstituted —C₅₋₁₄ cycloalkenyl, and substituted or unsubstituted —C₈₋₁₄ cycloalkynyl;
provided that: when R^{a} is —OH, L¹ is absent; when R^{a} is ═O, L¹ and R^{d1} are absent; when R^{b} is —OH, L² is absent; when R^{b} is ═O, L² is absent, R^{d2} is absent, and the C2-C3 bond and the C3-C4 bond are single bonds; when R^{c} is —OH, LU is absent; when R^{c} is ═O, L³ and R^{d3} are absent;
when R^{a}, R^{b} or R^{c} is substituted, R^{a}, R^{b} or R^{c} is substituted with one or more groups selected from Substituent Group A;
R^{d1}, when present, is H or is a group that, together with R^{a}, forms a substituted or unsubstituted ozonide ring or a substituted or unsubstituted tetraoxane ring, wherein the optional substituent may be a —C₃₋₁₀ cycloalkyl; provided that when R^{d1} and R^{a} form a substituted or unsubstituted ozonide ring or a substituted or unsubstituted tetraoxane ring, L¹ is absent; wherein, when the ozonide or tetraoxane ring is substituted, the ozonide or tetraoxane ring is substituted with one or more —C₃₋₁₀ cycloalkyl groups;
R^{d2}, when present, is H; provided that when R^{d2} is present, the C2-C3 bond and the C3-C4 bond are single bonds;
R^{d3}, when present, is H or is a group that, together with R^{c}, forms a substituted or unsubstituted ozonide ring or a substituted or unsubstituted tetraoxane ring, wherein the optional substituent may be a —C₃₋₁₀ cycloalkyl; provided that when R^{d3} and R^{c} form a substituted or unsubstituted ozonide ring or a substituted or unsubstituted tetraoxane ring, L³ is absent; wherein, when the ozonide or tetraoxane ring is substituted, the ozonide or tetraoxane ring is substituted with one or more —C₃₋₁₀ cycloalkyl groups;
R^{e} is H or CH₃, or R^{e} is absent;
when present, L¹ is a group that provides a covalent linkage between R^{a} and the C17 of ring D;
when present, L² is a group that provides a covalent linkage between R^{b} and the C3 of ring A;
when present, L² is a group that provides a covalent linkage between R^{c} and the C16 of ring D;
ring A may be a saturated or unsaturated ring, or ring A may be an aromatic ring, provided that when ring A is an aromatic ring, the C5-C6 bond is a single bond, R^{d2} is absent, R^{e} is absent, and R^{b} is not ═O; [symbol]
is a single bond or a double bond; provided that when the C4-C5 or C5-C10 bond is a double bond, the C5-C6 bond is a single bond; when the C5-C6 bond is a double bond, the C4-C5 and C5-C10 bonds are single bonds; and when the C3 forms a double bond with C2 or C4, R^{b} is not ═O;
or a stereoisomer thereof, or a salt thereof.

69. A method according to claim 68, wherein the parasite is a protozoan parasite and the anti-parasite moiety is an anti-protozoan moiety, or the parasite is a helminth and the anti-parasite moiety is an anti-helminth moiety, or the parasite is an apicomplexan parasite and the anti-parasite moiety is an anti-apicomplexan moiety.

70. A compound according to claim 67, wherein the compound is a compound of Formula (I″): 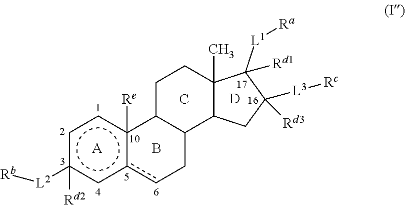
wherein:
each of R^{a} and R^{c} is independently selected from —H, —OH, ═O, substituted or unsubstituted —C₁₋₁₀ alkyl, substituted or unsubstituted —C₂₋₁₀ alkenyl, substituted or unsubstituted —C₂₋₁₀ alkynyl, substituted or unsubstituted —C₃₋₁₀ cycloalkyl, substituted or unsubstituted —C₅₋₁₄ cycloalkenyl, substituted or unsubstituted —C₈₋₁₄ cycloalkynyl, and an anti-parasite moiety; wherein at least one of R^{a} and R^{c} is an anti-parasite moiety;
R^{b} is selected from —H, —OH, ═O, substituted or unsubstituted —C₁₋₁₀ alkyl, substituted or unsubstituted —C₂₋₁₀ alkenyl, substituted or unsubstituted —C₂₋₁₀ alkynyl, substituted or unsubstituted —C₃₋₁₀ cycloalkyl, substituted or unsubstituted —C₅₋₁₄ cycloalkenyl, and substituted or unsubstituted —C₈₋₁₄ cycloalkynyl;
provided that: when R^{a} is —OH, L¹ is absent; when R^{a} is ═O, L¹ and R^{d1} are absent; when R^{b} is —OH, L² is absent; when R^{b} is ═O, L² is absent, R^{d2} is absent, and the C2-C3 bond and the C3-C4 bond are single bonds; when R^{c} is —OH, L³ is absent; when R^{c} is ═O, L³ and R^{d3} are absent;
when R^{a}, R^{b} or R^{c} is substituted, R^{a}, R^{b} or R^{c} is substituted with one or more groups selected from Substituent Group A;
R^{d1}, when present, is H or is a group that, together with R^{a}, forms a substituted or unsubstituted ozonide ring or a substituted or unsubstituted tetraoxane ring, wherein the optional substituent may be a —C₃₋₁₀ cycloalkyl; provided that when R^{d1} and R^{a} form a substituted or unsubstituted ozonide ring or a substituted or unsubstituted tetraoxane ring, L¹ is absent; wherein, when the ozonide or tetraoxane ring is substituted, the ozonide or tetraoxane ring is substituted with one or more —C₃₋₁₀ cycloalkyl groups;
R^{d2}, when present, is H; provided that when R^{d2} is present, the C2-C3 bond and the C3-C4 bond are single bonds;
R^{d3}, when present, is H or is a group that, together with R^{c}, forms a substituted or unsubstituted ozonide ring or a substituted or unsubstituted tetraoxane ring, wherein the optional substituent may be a —C₃₋₁₀ cycloalkyl; provided that when R^{d3} and R^{c} form a substituted or unsubstituted ozonide ring or a substituted or unsubstituted tetraoxane ring, L³ is absent; wherein, when the ozonide or tetraoxane ring is substituted, the ozonide or tetraoxane ring is substituted with one or more —C₃₋₁₀ cycloalkyl groups;
R^{e} is H or CH₃, or R^{e} is absent;
when present, L¹ is a group that provides a covalent linkage between R^{a} and the C17 of ring D;
when present, L² is a group that provides a covalent linkage between R^{b} and the C3 of ring A;
when present, L³ is a group that provides a covalent linkage between R^{c} and the C16 of ring D;
ring A may be a saturated or unsaturated ring, or ring A may be an aromatic ring, provided that when ring A is an aromatic ring, the C5-C6 bond is a single bond, R^{d2} is absent, R^{e} is absent, and R^{b} is not ═O; [symbol]
is a single bond or a double bond; provided that when the C4-C5 or C5-C10 bond is a double bond, the C5-C6 bond is a single bond; when the C5-C6 bond is a double bond, the C4-C5 and C5-C10 bonds are single bonds; and when the C3 forms a double bond with C2 or C4, R^{b} is not ═O;
or a stereoisomer thereof, or a salt thereof;
wherein the anti-parasite moiety is selected from: 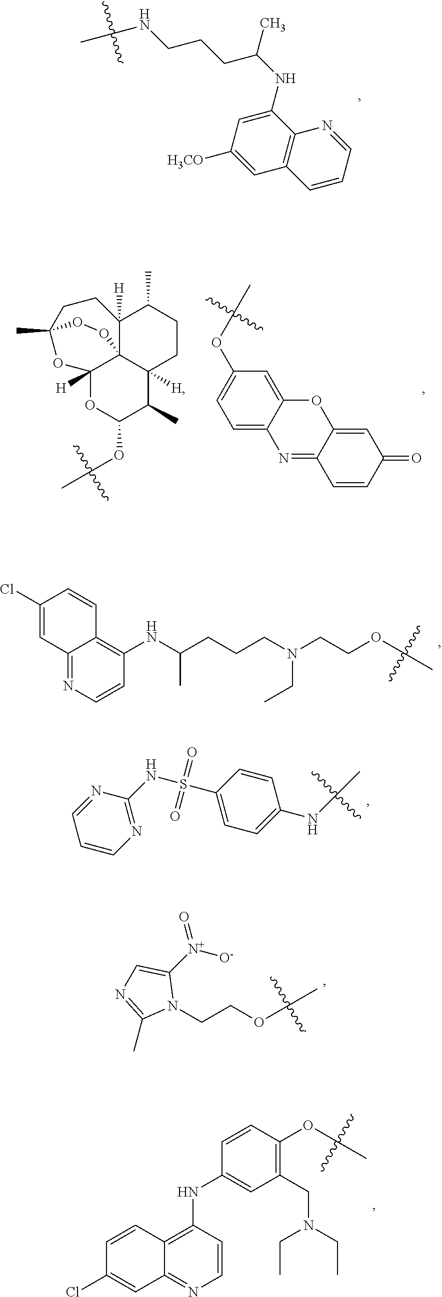 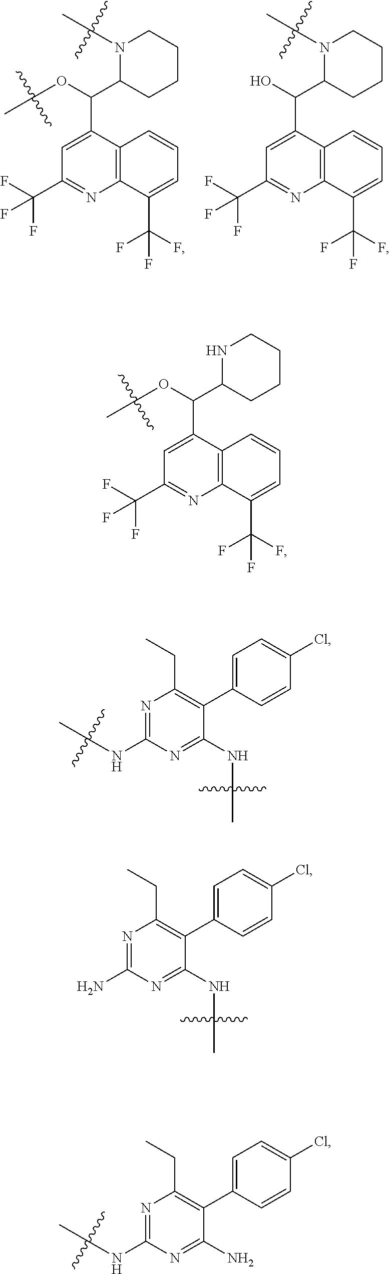 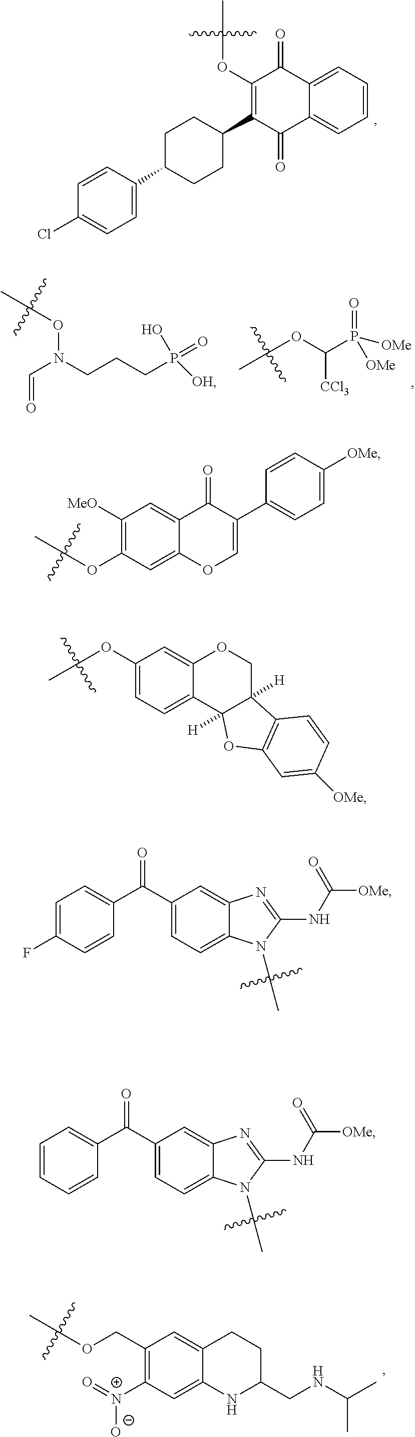 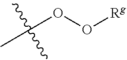
wherein R^{g} is H or substituted or unsubstituted —C₁₋₆ alkyl, 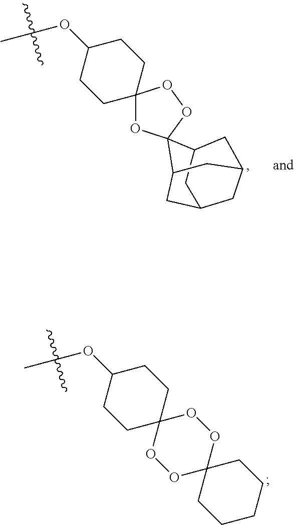 [SMILES: *OC1CCC2(CC1)OOC1(O2)C2CC3CC(C2)CC1C3 ; *OC1CCC2(CC1)OOC1(CCCCC1)OO2]
and
wherein each of L¹, L² and L³, when present, is substituted or unsubstituted and is independently selected from: 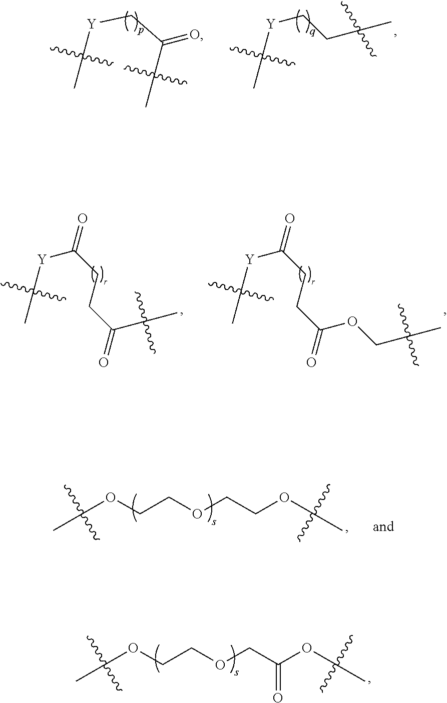
wherein Y is —NR^{f}—, —S—, —O— or —CR^{f1}R^{f2}—, wherein each of R^{f}, R^{f1} and R^{f2} is H or substituted or unsubstituted —C₁₋₆ alkyl; each of p, q, r and s is independently 0, 1, 2, 3 or 4; wherein, when L¹, L² or L³ is substituted, L¹, L² or L³ is substituted with one or more groups selected from Substituent Group A.

71. A compound according to claim 70, wherein Formula (I″) is selected from:

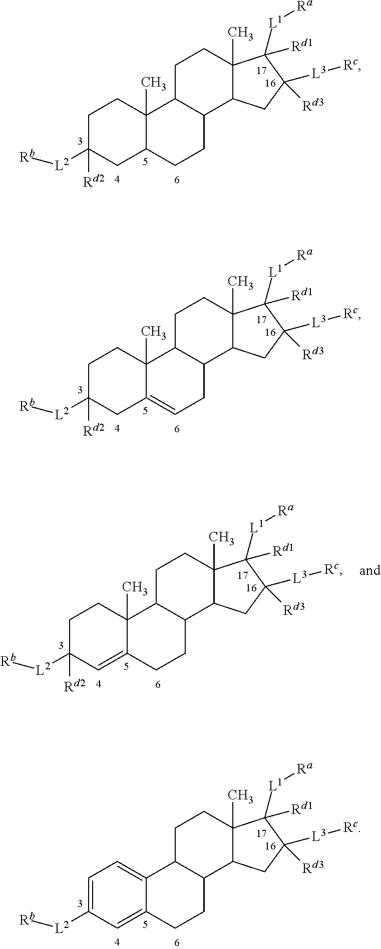

72. A compound according to claim 70, wherein Formula (I″) is selected from:

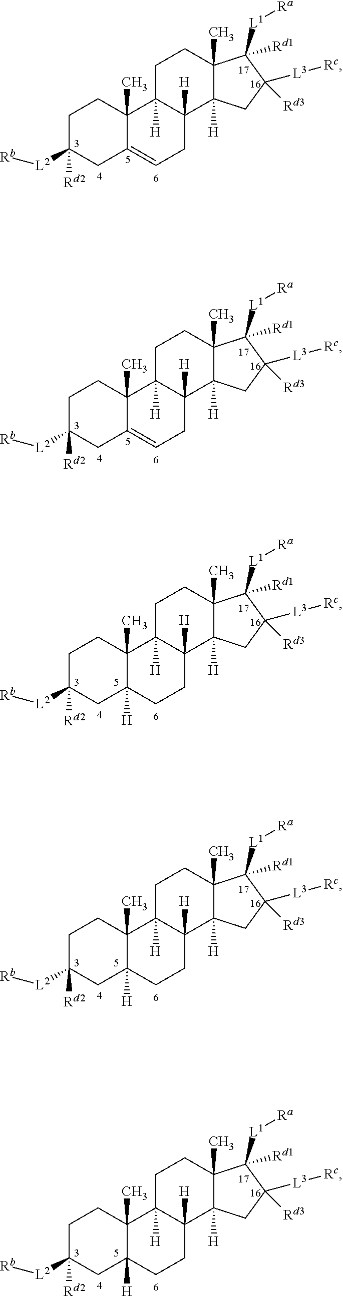

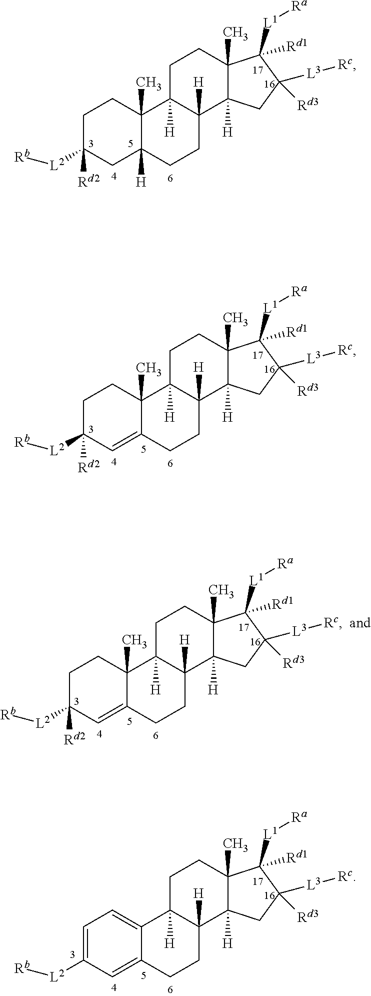

73. A compound according to claim 70, wherein each of L¹, L², and L³, when present, is substituted or unsubstituted and is independently selected from:

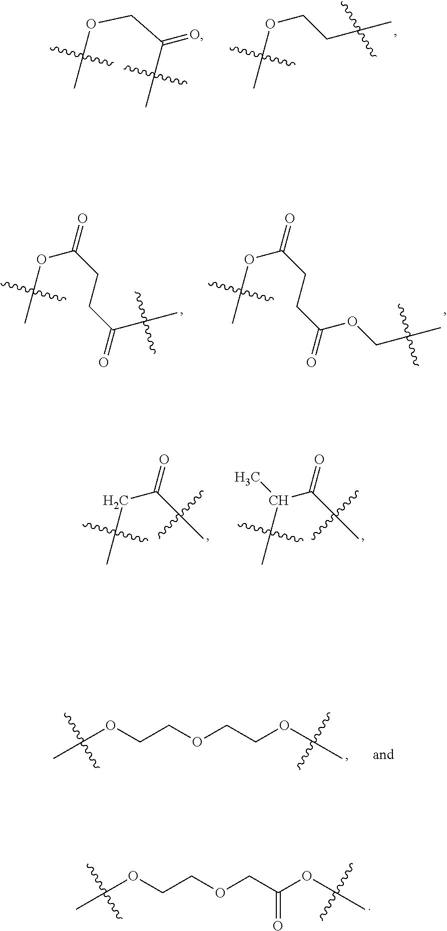

74. A compound according to claim 70 selected from: 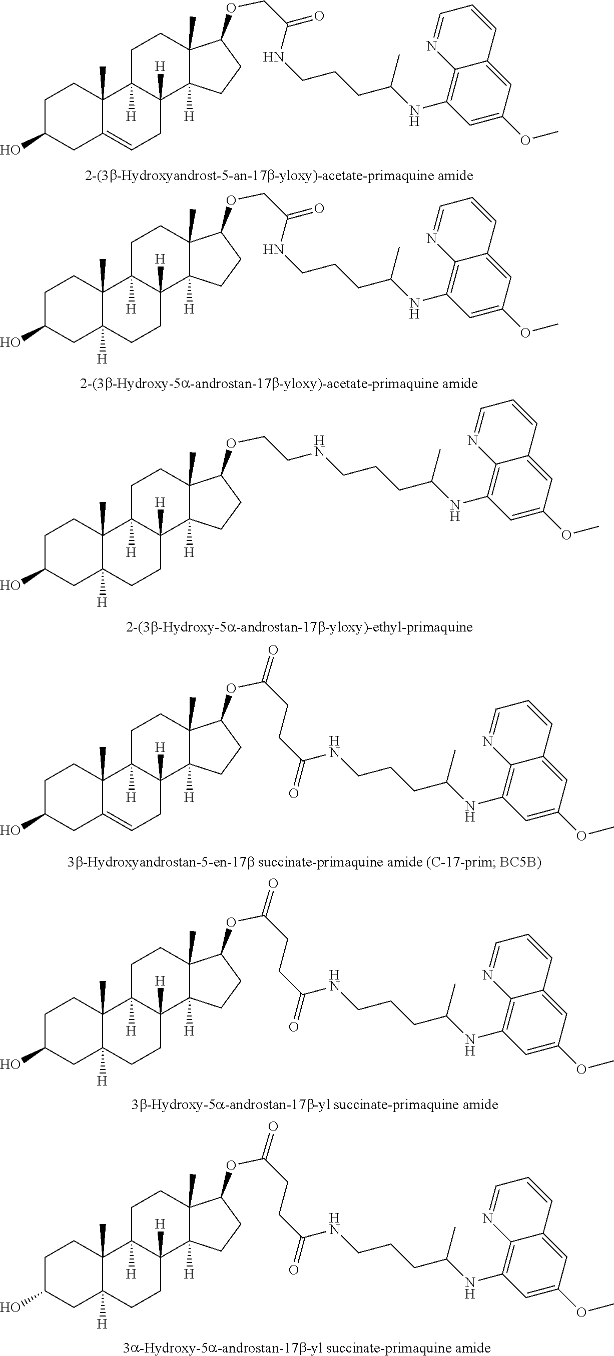 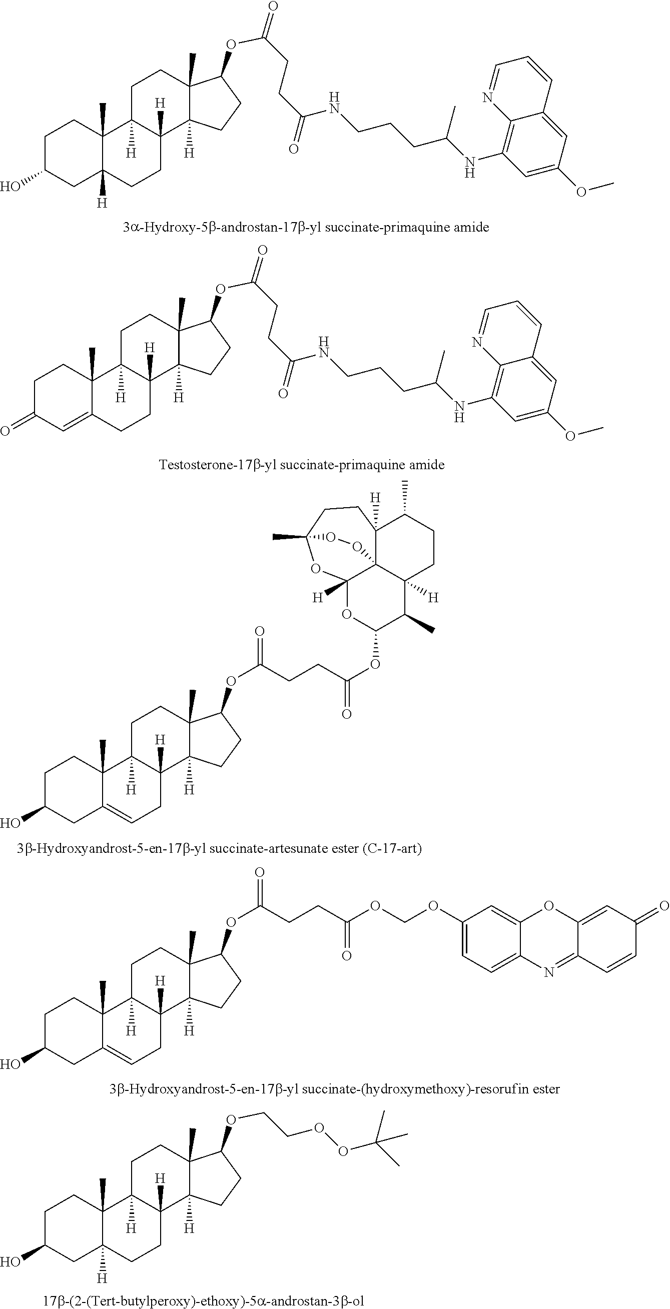 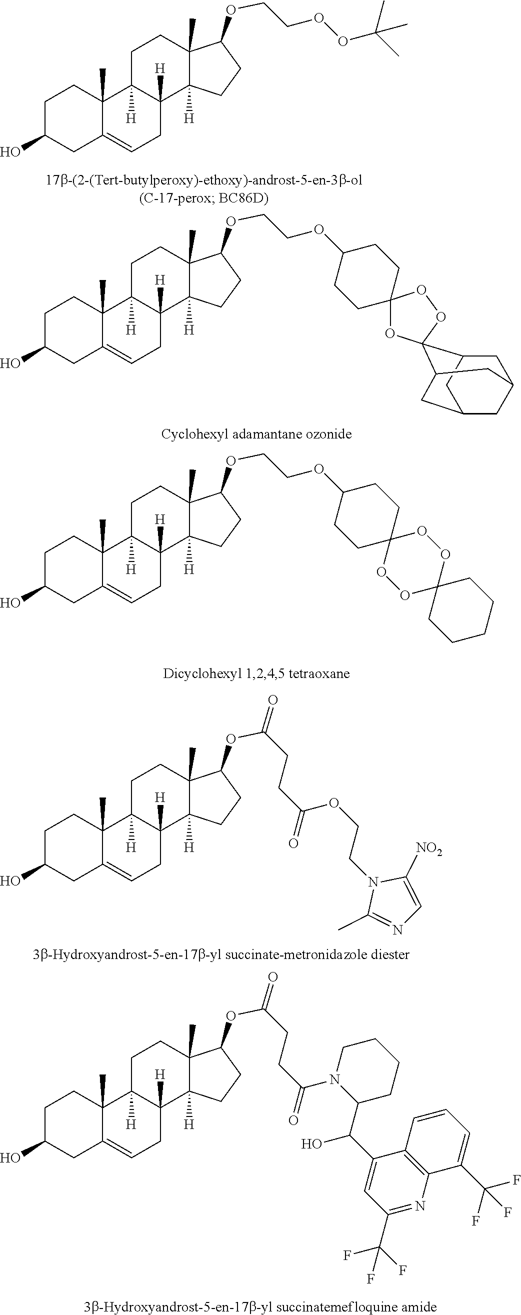 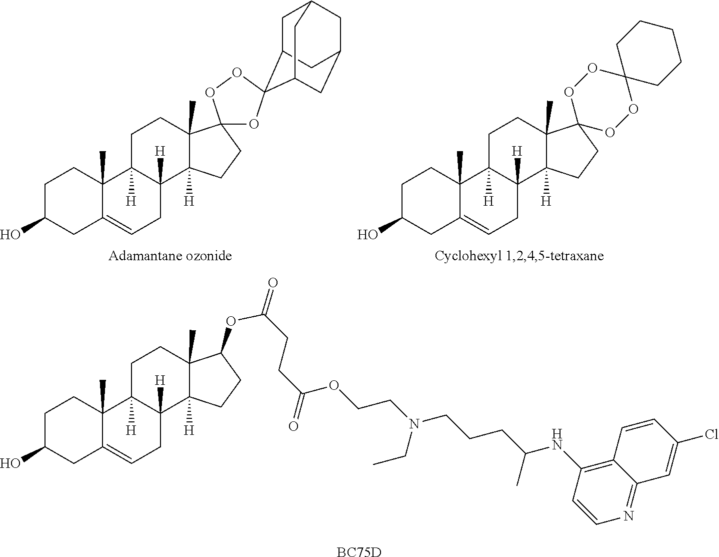
or a salt thereof.

75. A composition comprising a compound of Formula (I″) according to claim 70, or a salt thereof, and a suitable carrier, adjuvant or diluent.

76. A composition according to claim 75, wherein the composition is a pharmaceutical composition comprising a pharmaceutically acceptable carrier, adjuvant or diluent.
